SEQUENCE LISTING (224 sequences)

SEQ ID NO: 1 - protein, 4 aa, synthetic construct
KERE

SEQ ID NO: 2 - protein, 4 aa, synthetic construct
KQRE

SEQ ID NO: 3 - protein, 5 aa, synthetic construct
KEREL

SEQ ID NO: 4 - protein, 5 aa, synthetic construct
KEREF

SEQ ID NO: 5 - protein, 5 aa, synthetic construct
KQREL

SEQ ID NO: 6 - protein, 5 aa, synthetic construct
KQREF

SEQ ID NO: 7 - protein, 5 aa, synthetic construct
KEREG

SEQ ID NO: 8 - protein, 5 aa, synthetic construct
KQREW

SEQ ID NO: 9 - protein, 5 aa, synthetic construct
KQREG

SEQ ID NO: 10 - protein, 4 aa, synthetic construct
TERE

SEQ ID NO: 11 - protein, 5 aa, synthetic construct
TEREL

SEQ ID NO: 12 - protein, 4 aa, synthetic construct
TQRE

SEQ ID NO: 13 - protein, 5 aa, synthetic construct
TQREL

SEQ ID NO: 14 - protein, 4 aa, synthetic construct
KECE

SEQ ID NO: 15 - protein, 5 aa, synthetic construct
KECEL

SEQ ID NO: 16 - protein, 5 aa, synthetic construct
KECER

SEQ ID NO: 17 - protein, 4 aa, synthetic construct
KQCE

SEQ ID NO: 18 - protein, 5 aa, synthetic construct
KQCEL

SEQ ID NO: 19 - protein, 4 aa, synthetic construct
RERE

SEQ ID NO: 20 - protein, 5 aa, synthetic construct
REREG

SEQ ID NO: 21 - protein, 4 aa, synthetic construct
RQRE

SEQ ID NO: 22 - protein, 5 aa, synthetic construct
RQREL

SEQ ID NO: 23 - protein, 5 aa, synthetic construct
RQREF

SEQ ID NO: 24 - protein, 5 aa, synthetic construct
RQREW

SEQ ID NO: 25 - protein, 4 aa, synthetic construct
QERE

SEQ ID NO: 26 - protein, 5 aa, synthetic construct
QEREG

SEQ ID NO: 27 - protein, 4 aa, synthetic construct
QQRE

SEQ ID NO: 28 - protein, 5 aa, synthetic construct
QQREW

SEQ ID NO: 29 - protein, 5 aa, synthetic construct
QQREL

SEQ ID NO: 30 - protein, 5 aa, synthetic construct
QQREF

SEQ ID NO: 31 - protein, 4 aa, synthetic construct
KGRE

SEQ ID NO: 32 - protein, 5 aa, synthetic construct
KGREG

SEQ ID NO: 33 - protein, 4 aa, synthetic construct
KDRE

SEQ ID NO: 34 - protein, 5 aa, synthetic construct
KDREV

SEQ ID NO: 35 - protein, 5 aa, synthetic construct
DECKL

SEQ ID NO: 36 - protein, 5 aa, synthetic construct
NVCEL

SEQ ID NO: 37 - protein, 4 aa, synthetic construct
GLEW

SEQ ID NO: 38 - protein, 4 aa, synthetic construct
GVEW

SEQ ID NO: 39 - protein, 4 aa, synthetic construct
EPEW

SEQ ID NO: 40 - protein, 4 aa, synthetic construct
GLER

SEQ ID NO: 41 - protein, 4 aa, synthetic construct
DQEW

SEQ ID NO: 42 - protein, 4 aa, synthetic construct
DLEW

SEQ ID NO: 43 - protein, 4 aa, synthetic construct
GIEW

SEQ ID NO: 44 - protein, 4 aa, synthetic construct
ELEW

SEQ ID NO: 45 - protein, 4 aa, synthetic construct
GPEW

SEQ ID NO: 46 - protein, 4 aa, synthetic construct
EWLP

SEQ ID NO: 47 - protein, 4 aa, synthetic construct
GPER

SEQ ID NO: 48 - protein, 4 aa, synthetic construct
GLER

SEQ ID NO: 49 - protein, 4 aa, synthetic construct
ELEW

SEQ ID NO: 50 - protein, 115 aa, synthetic construct [sequence omitted]

SEQ ID NO: 51 - protein, 115 aa, synthetic construct [sequence omitted]

SEQ ID NO: 52 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 53 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 54 - protein, 115 aa, synthetic construct [sequence omitted]

SEQ ID NO: 55 - protein, 116 aa, synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 56 - protein, 115 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 57 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 58 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 59 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 60 - protein, 116 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 61 - protein, 117 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 62 - protein, 118 aa, synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 63 - protein, 115 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 64 - protein, 116 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 65 - protein, 10 aa, synthetic construct
GFTFRSFGMS

SEQ ID NO: 66 - protein, 10 aa, synthetic construct
SISGSGSDTL

SEQ ID NO: 67 - protein, 6 aa, synthetic construct
GGSLSR

SEQ ID NO: 68 - protein, 155 aa, synthetic construct
DLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISIDNGNEDLAEILQK

SEQ ID NO: 69 - protein, 25 aa, synthetic construct
EVQLVESGGGVVQPGGSLRLSCAAS

SEQ ID NO: 70 - protein, 14 aa, synthetic construct
WVRQAPGKGPEWVS

SEQ ID NO: 71 - protein, 39 aa, synthetic construct
YADSVKGRFTISRDNSKNTLYLQMNSLRPEDTALYYCTI

SEQ ID NO: 72 - protein, 11 aa, synthetic construct
SSQGTLVTVSS

SEQ ID NO: 73 - protein, 30 aa, synthetic construct
EVQLVESGGGVVQPGGSLRLSCAASGFTFR

SEQ ID NO: 74 - protein, 5 aa, synthetic construct
SFGMS

SEQ ID NO: 75 - protein, 14 aa, synthetic construct
WVRQAPGKGPEWVS

SEQ ID NO: 76 - protein, 17 aa, synthetic construct
SISGSGSDTLYADSVKG

SEQ ID NO: 77 - protein, 32 aa, synthetic construct
RFTISRDNSKNTLYLQMNSLRPEDTALYYCTI

SEQ ID NO: 78 - protein, 6 aa, synthetic construct
GGSLSR

SEQ ID NO: 79 - protein, 11 aa, synthetic construct
SSQGTLVTVSS

SEQ ID NO: 80 - protein, 126 aa, synthetic construct
EVQLVESGGGLVQAGGSLSISCAASGGSLSNYVLGWFRQAPGCEREFVAAINWRGDITIGPPNVEGRFTISRDNAKNTGYLQMNSLAPDDTAVYYCGAGTPLNPGAYIYCWSYDYWGCGTLVTVSS

SEQ ID NO: 81 - protein, 126 aa, synthetic construct
EVQLVESGGGLVQAGGSLSISCAASGGSLSNYVLGWFRQAPGCEREFVAAINWRGDITIGPPNVEGRFTISRDNACNTGYLQMNSLAPDDTAVYYCGAGTPLNPCAYIYDWSYDYWGRGTLVTVSS

SEQ ID NO: 82 - protein, 126 aa, synthetic construct
EVQLVESGGGLVQAGGSLSICCAASGGSLSNYVLGWFRQAPGKEREFVAAINWRGDITIGPPNVEGRFCISRDNAKNTGYLQMNSLAPDDTAVYYCGAGTPLNPGAYIYCWSYDYWGRGTLVTVSS

SEQ ID NO: 83 - protein, 126 aa, synthetic construct
EVQLVESGGGLVCAGGSLSISCAASGGSLSNYVLGWFRQAPGKEREFVAAINWRGDITIGPPNVEGRFTISRCNAKNTGYLQMNSLAPDDTAVYYCGAGTPLNPCAYIYDWSYDYWGRGTLVTVSS

SEQ ID NO: 84 - protein, 126 aa, synthetic construct
EVQLVESGGGLVCAGGSLSISCAASGGSLSCYVLGWFRQAPGKEREFVAAINWRGDITIGPPNVEGRFTISRDNAKNTGYLQMNSLAPDDTAVYYCGAGTPLNPGAYIYCWSYDYWGRGTLVTVSS

SEQ ID NO: 85 - protein, 126 aa, synthetic construct
EVQLVECGGGLVQAGGSLSISCAASGGSLSNYVLGWFRQAPGKCREFVAAINWRGCITIGPPNVEGRFTISRDNAKNTGYLQMNSLAPDDTAVYYCGAGTPLNPGAYIYDWSYDYWGRGTLVTVSS

SEQ ID NO: 86 - protein, 126 aa, synthetic construct
EVQLVESGGGLVQAGGSLCISCAASGGSLSNYVLGWFRQAPGKCREFVAAINWRGDITIGPPNVECRFTICRDNAKNTGYLQMNCLAPDDTAVYYCGAGTPLNPGAYIYDWSYDYWGRGTLVTVCS

SEQ ID NO: 87 - protein, 126 aa, synthetic construct
EVQLVESGGGLVQAGGSLSICCAASGGSLSNYVLGWFRQAPGCEREFVAAINWRGCITIGPPNVEGRFCISRDNCKNTGYLQMNSLAPDDTAVYYCGAGTPLNPGAYIYDWSYDYWGRGTLVTVCS

SEQ ID NO: 88 - protein, 126 aa, synthetic construct
EVQLVESGGGLVQAGGSLCISCCASGGSLSCYVLGWFRQAPGKEREFVAAINWRGDITIGPPNVEGRFTICRDNAKNTGYLQMNCLAPDDTAVYYCGAGTPLNPGAYIYCWSYDYWGRGTLVTVSS

SEQ ID NO: 89 - protein, 126 aa, synthetic construct
EVQLVESGGGLVCAGGSLSISCAACGGSLSNYVLGWFRQAPGCEREFVAAINWRGDITIGPPNVECRFTISRCNAKNTGYLQMNSLAPDDTAVYYCGAGTPLNPCAYIYDWSYDYWGRGTLVTVSS

SEQ ID NO: 90 - protein, 126 aa, synthetic construct
EVQLVESGGGVVQAGGSLSISCAACGGSLSNYVLGWFRQAPGCEREFVAAINWRGDITIGPPNVEGRFTISRDNACNTGYLQMNCLAPDDTALYYCGAGTPLNPCAYIYDWSYDYWGRGTLVTVCS

SEQ ID NO: 91 - protein, 126 aa, synthetic construct
EVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPCAYIYDWSYDYWGCGTLVTVCS

SEQ ID NO: 92 - protein, 129 aa, synthetic construct
EVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPCAYIYDWSYDYWGCGTLVTVSSGGC

SEQ ID NO: 93 - protein, 129 aa, synthetic construct
EVQLVESGGGVVQAGGSLSISCAASGGSLSNYVLGWFRQAPGCEREFVAAINWRGDITIGPPNVEGRFCISRDNACNTGYLQMNSLAPDDTALYYCGAGTPLNPCAYIYDWSYDYWGCGTLVTVSSGGC

SEQ ID NO: 94 - protein, 129 aa, synthetic construct
EVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPGAYIYCWSYDYWGCGTLVTVSSGGC

SEQ ID NO: 95 - protein, 129 aa, synthetic construct
EVQLVESGGGVVQAGGSLSISCAASGGSLSNYVLGWFRQAPGCEREFVAAINWRGDITIGPPNVEGRFCISRDNACNTGYLQMNSLAPDDTALYYCGAGTPLNPGAYIYCWSYDYWGCGTLVTVSSGGC

SEQ ID NO: 96 - protein, 156 aa, synthetic construct
DLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISICNGNECLAEILQKC

SEQ ID NO: 97 - protein, 156 aa, synthetic construct
DLGKKLLEAARAGQDDEVRILMANGADVNAHDTFGFTPLHLAALYGHLEIVEVLLKNGADVNADDSYGATPLHLAAMRGHLEIVCVLLKYGADVCAADEEGATPLHLAAKAGHLEIVEVLLKNGADVNAQDKFGKTAFDISIDNGNEDLAEILQKC

SEQ ID NO: 98 - protein, 156 aa, synthetic construct
DLGKKLLEAARAGQDDEVRILMANGADVNAHDTFGFTPLHLAALYGHLEIVEVLLKNGADVNADDSYGATPLHLAAMRGHLEIVCVLLKYGADVCAADEEGATPLHLAAKAGHLEIVEVLLKNGADVNAQDKFGKTAFDISICNGNECLAEILQKC

SEQ ID NO: 99 - protein, 78 aa, synthetic construct
SHKQIYYSDCYDCEEFEYRHVMLPKDIAKLVPKTHLMSESEWRNLGVQQSQGWVHYMIHEPEPHILLFRRPLPKKPKC

SEQ ID NO: 100 - protein, 78 aa, synthetic construct
SHKQIYYSDCYCDEEFEYRHVCLPKDIAKLVPKTHLMSESEWRNLGVQQSCGWVHYMIHEPEPHILLFRRPLPKKPKK

SEQ ID NO: 101 - protein, 78 aa, synthetic construct
SHKQIYYSDKCDDEEFEYRHVMLPKDIAKLVPKTHLMSESEWRNLGVQQSCGWVHYMIHEPEPHILLFRRPLPKKPKC

SEQ ID NO: 102 - protein, 78 aa, synthetic construct
SHKQIYYSDKYDCEEFEYRHVMLPKDIAKLVPCTHLMSESEWRNLGVQQSQGWVHYMIHEPEPHILLFRRPLPKKPKC

SEQ ID NO: 103 - protein, 78 aa, synthetic construct
SHKQIYYSDKCDDEEFEYRHVMLPKDIAKLVPCTHLMSESEWRNLGVQQSCGWVHYMIHEPEPHILLFRRPLPKKPKC

SEQ ID NO: 104 - protein, 78 aa, synthetic construct
SHKQIYYSCKYDCEEFEYRHVCLPKDIAKLVPCTHLMSESEWRNLGVQQSCGWVHYMIHEPEPHILLFRRPLPKKPKC

SEQ ID NO: 105 - protein, 73 aa, synthetic construct
SHKQIYYSCKYDCEEFEYRHVMLPKDIAKLVPCTHLMSESEWRNLGVQQSCGWVHYCIHEPEPHILLFRRPLC

SEQ ID NO: 106 - protein, 115 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 107 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLSISCAASGGSLSNYVLGWFRQAPGCEREFVAAINWRGDITIGPPNVEGRFTISRDNAKNTGYLQMNSLAPDDTAVYYCGAGTPLNPGAYIYCWSYDYWGCGTLVTVSS

SEQ ID NO: 108 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLSISCAASGGSLSNYVLGWFRQAPGCEREFVAAINWRGDITIGPPNVEGRFTISRDNACNTGYLQMNSLAPDDTAVYYCGAGTPLNPCAYIYDWSYDYWGRGTLVTVSS

SEQ ID NO: 109 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLSICCAASGGSLSNYVLGWFRQAPGKEREFVAAINWRGDITIGPPNVEGRFCISRDNAKNTGYLQMNSLAPDDTAVYYCGAGTPLNPGAYIYCWSYDYWGRGTLVTVSS

SEQ ID NO: 110 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVCAGGSLSISCAASGGSLSNYVLGWFRQAPGKEREFVAAINWRGDITIGPPNVEGRFTISRCNAKNTGYLQMNSLAPDDTAVYYCGAGTPLNPCAYIYDWSYDYWGRGTLVTVSS

SEQ ID NO: 111 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVCAGGSLSISCAASGGSLSCYVLGWFRQAPGKEREFVAAINWRGDITIGPPNVEGRFTISRDNAKNTGYLQMNSLAPDDTAVYYCGAGTPLNPGAYIYCWSYDYWGRGTLVTVSS

SEQ ID NO: 112 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVECGGGLVQAGGSLSISCAASGGSLSNYVLGWFRQAPGKCREFVAAINWRGCITIGPPNVEGRFTISRDNAKNTGYLQMNSLAPDDTAVYYCGAGTPLNPGAYIYDWSYDYWGRGTLVTVSS

SEQ ID NO: 113 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLCISCAASGGSLSNYVLGWFRQAPGKCREFVAAINWRGDITIGPPNVECRFTICRDNAKNTGYLQMNCLAPDDTAVYYCGAGTPLNPGAYIYDWSYDYWGRGTLVTVCS

SEQ ID NO: 114 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLSICCAASGGSLSNYVLGWFRQAPGCEREFVAAINWRGCITIGPPNVEGRFCISRDNCKNTGYLQMNSLAPDDTAVYYCGAGTPLNPGAYIYDWSYDYWGRGTLVTVCS

SEQ ID NO: 115 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLCISCCASGGSLSCYVLGWFRQAPGKEREFVAAINWRGDITIGPPNVEGRFTICRDNAKNTGYLQMNCLAPDDTAVYYCGAGTPLNPGAYIYCWSYDYWGRGTLVTVSS

SEQ ID NO: 116 - protein, 256 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVCAGGSLSISCAACGGSLSNYVLGWFRQAPGCEREFVAAINWRGDITIGPPNVECRFTISRCNAKNTGYLQMNSLAPDDTAVYYCGAGTPLNPCAYIYDWSYDYWGRGTLVTVSS

SEQ ID NO: 117 - protein, 256 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAACGGSLSNYVLGWFRQAPGCEREFVAAINWRGDITIGPPNVEGRFTISRDNACNTGYLQMNCLAPDDTALYYCGAGTPLNPCAYIYDWSYDYWGRGTLVTVCS

SEQ ID NO: 118 - protein, 256 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPCAYIYDWSYDYWGCGTLVTVCS

SEQ ID NO: 119 - protein, 259 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPCAYIYDWSYDYWGCGTLVTVSSGGC

SEQ ID NO: 120 - protein, 259 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAASGGSLSNYVLGWFRQAPGCEREFVAAINWRGDITIGPPNVEGRFCISRDNACNTGYLQMNSLAPDDTALYYCGAGTPLNPCAYIYDWSYDYWGCGTLVTVSSGGC

SEQ ID NO: 121 - protein, 259 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAACGGSLSNYVLGWFRQAPGCEREFVAAINWRGDITIGPPNVEGRFTISRDNACNTGYLQMNSLAPDDTALYYCGAGTPLNPGAYIYCWSYDYWGCGTLVTVSSGGC

SEQ ID NO: 122 - protein, 259 aa, synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGVVQAGGSLSISCAASGGSLSNYVLGWFRQAPGCEREFVAAINWRGDITIGPPNVEGRFCISRDNACNTGYLQMNSLAPDDTALYYCGAGTPLNPGAYIYCWSYDYWGCGTLVTVSSGGC

SEQ ID NO: 123 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDCYDCEEFEYRHVMLPKDIAKLVPKTHLMSESEWRNLGVQQSQGWVHYMIHEPEPHILLFRRPLPKKPKC

SEQ ID NO: 124 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDCYCDEEFEYRHVCLPKDIAKLVPKTHLMSESEWRNLGVQQSCGWVHYMIHEPEPHILLFRRPLPKKPKK

SEQ ID NO: 125 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDKCDDEEFEYRHVMLPKDIAKLVPKTHLMSESEWRNLGVQQSCGWVHYMIHEPEPHILLFRRPLPKKPKC

SEQ ID NO: 126 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDKYDCEEFEYRHVMLPKDIAKLVPCTHLMSESEWRNLGVQQSQGWVHYMIHEPEPHILLFRRPLPKKPKC

SEQ ID NO: 127 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDKCDDEEFEYRHVMLPKDIAKLVPCTHLMSESEWRNLGVQQSCGWVHYMIHEPEPHILLFRRPLPKKPKC

SEQ ID NO: 128 - protein, 30 aa, synthetic construct
EVQLVESGGGLVQAGGSLSISCAASGGSLS

SEQ ID NO: 129 - protein, 30 aa, synthetic construct
DVQLVESGGGLVQAGGSLSISCAASGGSLS

SEQ ID NO: 130 - protein, 30 aa, synthetic construct
EVQLLESGGGLVQPGGSLRLSCAASGGSLS

SEQ ID NO: 131 - protein, 30 aa, synthetic construct
EVQLLESGGGLVQPGGSLRISCAASGGSLS

SEQ ID NO: 132 - protein, 30 aa, synthetic construct
DVQLLESGGGLVQPGGSLRISCAASGGSLS

SEQ ID NO: 133 - protein, 30 aa, synthetic construct
EVQLLESGGGLVQPGGSLSISCAASGGSLS

SEQ ID NO: 134 - protein, 30 aa, synthetic construct
DVQLLESGGGLVQPGGSLRLSCAASGGSLS

SEQ ID NO: 135 - protein, 30 aa, synthetic construct
EVQLLESGGGLVQAGGSLSISCAASGGSLS

SEQ ID NO: 136 - protein, 30 aa, synthetic construct
EVQLLESGGGLVQAGGSLRLSCAASGGSLS

SEQ ID NO: 137 - protein, 30 aa, synthetic construct
EVQLVESGGGLVQPGGSLRLSCAASGGSLS

SEQ ID NO: 138 - protein, 30 aa, synthetic construct
DVQLVESGGGLVQPGGSLRLSCAASGGSLS

SEQ ID NO: 139 - protein, 5 aa, synthetic construct
NYVLG

SEQ ID NO: 140 - protein, 14 aa, synthetic construct
WFRQAPGKEREFVA

SEQ ID NO: 141 - protein, 14 aa, synthetic construct
WFRQAPGKGREFVA

SEQ ID NO: 142 - protein, 17 aa, synthetic construct
AINWRGDITIGPPNVEG

SEQ ID NO: 143 - protein, 17 aa, synthetic construct
AINWRDDITIGPPNVEG

SEQ ID NO: 144 - protein, 128 aa, synthetic construct
NCEVQLVESGGGLVQAGGSLSISCAASGGSLSNYVLGWFRQAPGKEREFVAAINWRGDITIGPPNVEGRFTISRDNAKNTGYLQMNSLAPDDTAVYYCGAGTPLNPGAYIYDWSYDYWGRGTQVTVSS

SEQ ID NO: 145 - protein, 32 aa, synthetic construct
RFTISRDNAKNTLYLQMNSLAPEDTAVYYCGA

SEQ ID NO: 146 - protein, 32 aa, unidentified
RFTISRDNSKNTLYLQMNSLAPEDTAVYYCGA

SEQ ID NO: 147 - protein, 32 aa, synthetic construct
RFTISRDNSKNTLYLQMNSLRPEDTAVYYCGA

SEQ ID NO: 148 - protein, 32 aa, synthetic construct
RFTISRDNSKNTLYLQMNSLRPDDTAVYYCGA

SEQ ID NO: 149 - protein, 32 aa, synthetic construct
RFTISRDNAKNTLYLQMNSLRPEDTAVYYCGA

SEQ ID NO: 150 - protein, 32 aa, synthetic construct
RFTISRDNAKNTLYLQMNSLAPDDTAVYYCGA

SEQ ID NO: 151 - protein, 32 aa, synthetic construct
RFTISRDNAKNTGYLQMNSLAPEDTAVYYCGA

SEQ ID NO: 152 - protein, 32 aa, synthetic construct
RFTISRDNAKNTGYLQMNSLRPDDTAVYYCGA

SEQ ID NO: 153 - protein, 32 aa, synthetic construct
RFTISRDNAKNTGYLQMNSLRPEDTAVYYCGA

SEQ ID NO: 154 - protein, 17 aa, synthetic construct
GTPLNPGAYIYDWSYDY

SEQ ID NO: 155 - protein, 11 aa, synthetic construct
WGRGTQVTVSS

SEQ ID NO: 156 - protein, 11 aa, synthetic construct
WGQGTLVTVSS

SEQ ID NO: 157 - protein, 11 aa, synthetic construct
WGRGTLVTVSS

SEQ ID NO: 158 - protein, 5 aa, synthetic construct
GGGGS

SEQ ID NO: 159 - protein, 7 aa, synthetic construct
SGGSGGS

SEQ ID NO: 160 - protein, 8 aa, synthetic construct
GGGGGGGS

SEQ ID NO: 161 - protein, 9 aa, synthetic construct
GGGGSGGGS

SEQ ID NO: 162 - protein, 10 aa, synthetic construct
GGGGSGGGGS

SEQ ID NO: 163 - protein, 15 aa, synthetic construct
GGGGSGGGGSGGGGS

SEQ ID NO: 164 - protein, 18 aa, synthetic construct
GGGGSGGGGSGGGGGGGS

SEQ ID NO: 165 - protein, 20 aa, synthetic construct
GGGGSGGGGSGGGGSGGGGS

SEQ ID NO: 166 - protein, 25 aa, synthetic construct
GGGGSGGGGSGGGGSGGGGSGGGGS

SEQ ID NO: 167 - protein, 30 aa, synthetic construct
GGGGSGGGGSGGGGSGGGGSGGGGSGGGGS

SEQ ID NO: 168 - protein, 35 aa, synthetic construct
GGGGSGGGGSGGGGSGGGGSGGGGSGGGGSGGGGS

SEQ ID NO: 169 - protein, 40 aa, synthetic construct
GGGGSGGGGSGGGGSGGGGSGGGGSGGGGSGGGGSGGGGS

SEQ ID NO: 170 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSCKYDCEEFEYRHVCLPKDIAKLVPCTHLMSESEWRNLGVQQSCGWVHYMIHEPEPHILLFRRPLPKKPKC

SEQ ID NO: 171 - protein, 203 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSCKYDCEEFEYRHVMLPKDIAKLVPCTHLMSESEWRNLGVQQSCGWVHYCIHEPEPHILLFRRPLC

SEQ ID NO: 172 - protein, 286 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSDLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISICNGNECLAEILQKC

SEQ ID NO: 173 - protein, 286 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSDLGKKLLEAARAGQDDEVRILMANGADVNAHDTFGFTPLHLAALYGHLEIVEVLLKNGADVNADDSYGATPLHLAAMRGHLEIVCVLLKYGADVCAADEEGATPLHLAAKAGHLEIVEVLLKNGADVNAQDKFGKTAFDISIDNGNEDLAEILQKC

SEQ ID NO: 174 - protein, 286 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSDLGKKLLEAARAGQDDEVRILMANGADVNAHDTFGFTPLHLAALYGHLEIVEVLLKNGADVNADDSYGATPLHLAAMRGHLEIVCVLLKYGADVCAADEEGATPLHLAAKAGHLEIVEVLLKNGADVNAQDKFGKTAFDISICNGNECLAEILQKC

SEQ ID NO: 175 - protein, 129 aa, synthetic construct
EVQLVESGGGLVQAGGSLSISCAASGGSLSNYVLGWFRQAPGKEREFVAAINWRGDITIGPPNVEGRFTISRDNAKNTGYLQMNSLAPDDTAVYYCGAGTPLNPGAYIYDWSYDYWGRGTLVTVSSGGC

SEQ ID NO: 176 - protein, 259 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSEVQLVESGGGLVQAGGSLSISCAASGGSLSNYVLGWFRQAPGKEREFVAAINWRGDITIGPPNVEGRFTISRDNAKNTGYLQMNSLAPDDTAVYYCGAGTPLNPGAYIYDWSYDYWGRGTLVTVSSGGC

SEQ ID NO: 177 - protein, 129 aa, synthetic construct
DVQLVESGGGVVQPGGSLRLSCAASGRTFGSIRVGWFRQAPGKGREFVSAINRNDGTTYYADSVKGRFTISRDNSKNTVYLQMNSLRPEDTALYYCAAGLQYNRAADRVPVGAVYWGQGTLVTVSSGGC

SEQ ID NO: 178 - protein, 451 aa, synthetic construct
DVQLVESGGGVVQPGGSLRLSCAASGRTFGSIRVGWFRQAPGKGREFVSAINRNDGTTYYADSVKGRFTISRDNSKNTVYLQMNSLRPEDTALYYCAAGLQYNRAADRVPVGAVYWGQGTLVTVSSGGGGSGGGGSGGGGSGGGGSGGGGSGGGGSGGGGSEVQLVESGGGVVQPGGSLRLSCAASGRTFGSIRVGWFRQAPGKGREFVSAINRNDGTTYYADSVKGRFTISRDNSKNTVYLQMNSLRPEDTALYYCAAGLQYNRAADRVPVGAVYWGQGTLVTVSSGGGGSGGGGSGGGGSGGGGSGGGGSGGGGSGGGGSEVQLVESGGGVVQPGGSLRLSCAASGRTFGSIRVGWFRQAPGKGREFVSAINRNDGTTYYADSVKGRFTISRDNSKNTVYLQMNSLRPEDTALYYCAAGLQYNRAADRVPVGAVYWGQGTLVTVSSGGC

SEQ ID NO: 179 - protein, 126 aa, synthetic construct
EVQLVESGGGLVQAGGSLSISCAASGGSLSNYVLGWFRQAPGKEREFVAAINWRGDITIGPPNVEGRFTISRDNAKNTGYLQMNSLAPDDTAVYYCGAGTPLNPGAYIYDWSYDYWGRGTQVTVSS

SEQ ID NO: 180 - protein, 156 aa, synthetic construct
DLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISIDNGNEDLAEILQKL

SEQ ID NO: 181 - protein, 157 aa, synthetic construct
  VARIANT 85: note = X can be Glu or any amino acid with a reactive group in its side chain, such as cysteine
  VARIANT 95: note = X can be Asn or any amino acid with a reactive group in its side chain, such as cysteine
  VARIANT 143: note = X can be Asp or any amino acid with a reactive group in its side chain, such as cysteine
  VARIANT 148: note = X can be Asp or any amino acid with a reactive group in its side chain, such as cysteine
  VARIANT 156: note = absent or Leu
  VARIANT 157: note = absent or Cys
DLGKKLLEAARAGQDDEVRILMANGADVNAHDTFGFTPLHLAALYGHLEIVEVLLKNGADVNADDSYGATPLHLAAMRGHLEIVXVLLKYGADVXAADEEGATPLHLAAKAGHLEIVEVLLKNGADVNAQDKFGKTAFDISIXNGNEXLAEILQKXX

SEQ ID NO: 182 - protein, 156 aa, synthetic construct
  VARIANT 85: note = X can be Glu or any amino acid with a reactive group in its side chain, such as cysteine
  VARIANT 94: note = X can be Asn or any amino acid with a reactive group in its side chain, such as cysteine
  VARIANT 143: note = X can be Asp or any amino acid with a reactive group in its side chain, such as cysteine
  VARIANT 148: note = X can be Asp or any amino acid with a reactive group in its side chain, such as cysteine
DLGKKLLEAARAGQDDEVRILMANGADVNAHDTFGFTPLHLAALYGHLEIVEVLLKNGADVNADDSYGATPLHLAAMRGHLEIVXVLLKYGADVXAADEEGATPLHLAAKAGHLEIVEVLLKNGADVNAQDKFGKTAFDISIXNGNEXLAEILQKC

SEQ ID NO: 183 - protein, 129 aa, synthetic construct; identical to SEQ ID NO: 177

SEQ ID NO: 184 - protein, 451 aa, synthetic construct; identical to SEQ ID NO: 178

SEQ ID NO: 185 - protein, 129 aa, synthetic construct
  VARIANT 7: note = Ser or any amino acid with a reactive group in its side chain, such as cysteine
  VARIANT 11: note = Leu, or Val, Ser , Met, Trp, Phe, Thr, Gln, Glu, Ala, Arg, Gly, Lys, Tyr, Asn, Pro or Ile; preferably Leu, Val, Ser, or Glu, more preferably Leu or Val
  VARIANT 13: note = Gln or any amino acid with a reactive group in its side chain, such as cysteine
  VARIANT 17: note = Ser or any amino acid with a reactive group in its side chain, such as cysteine
  VARIANT 19: note = Ser or any amino acid with a reactive group in its side chain, such as cysteine
  VARIANT 21: note = Ser or any amino acid with a reactive group in its side chain, such as cysteine
  VARIANT 23: note = Ala or any amino acid with a reactive group in its side chain, such as cysteine
  VARIANT 25: note = Ser or any amino acid with a reactive group in its side chain, such as cysteine
  VARIANT 31: note = Asn or any amino acid with a reactive group in its side chain, such as cysteine
  VARIANT 43: note = Lys or any amino acid with a reactive group in its side chain, such as cysteine
  VARIANT 44: note = Glu or any amino acid with a reactive group in its side chain, such as cysteine
  VARIANT 56: note = Asp or any amino acid with a reactive group in its side chain, such as cysteine
  VARIANT 63: note = Asn or any amino acid with a reactive group in its side chain, such as cysteine
  VARIANT 66: note = Gly or any amino acid with a reactive group in its side chain, such as cysteine
  VARIANT 69: note = Thr or any amino acid with a reactive group in its side chain, such as cysteine
  VARIANT 71: note = Ser or any amino acid with a reactive group in its side chain, such as cysteine
  VARIANT 73: note = Asp or any amino acid with a reactive group in its side chain, such as cysteine
  VARIANT 75: note = Ala or any amino acid with a reactive group in its side chain, such as cysteine
  VARIANT 76: note = Lys or any amino acid with a reactive group in its side chain, such as cysteine
  VARIANT 85: note = Ser or any amino acid with a reactive group in its side chain, such as cysteine
  VARIANT 93: note = Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val
  VARIANT 105: note = Gly or any amino acid with a reactive group in its side chain, such as cysteine
  VARIANT 110: note = Asp or any amino acid with a reactive group in its side chain, such as cysteine
  VARIANT 118: note = Arg or any amino acid with a reactive group in its side chain, such as cysteine
  VARIANT 121: note = Gln, Leu, Arg, Pro, Glu, Lys, Ser, Thr, Met, Ala or His; preferably Gln or Leu
  VARIANT 125: note = Ser or any amino acid with a reactive group in its side chain, such as cysteine
  VARIANT 128: note = absent or Gly
  VARIANT 127: note = absent or Gly
  VARIANT 129: note = absent or Cys
EVQLVEXGGGZVXAGGXLXIXCXAXGGSLSXYVLGWFRQAPGXXREFVAAINWRGXITIGPPXVEXRFXIXRXNXXNTGYLQMNXLAPDDTAZYYCGAGTPLNPXAYIYXWSYDYWGXGTZVTVXSXXX

SEQ ID NO: 186 - protein, 129 aa, synthetic construct
  VARIANT 1: note = Glu or any amino acid with a reactive group in its side chain, such as cysteine
  VARIANT 3: note = Gln or any amino acid with a reactive group in its side chain, such as cysteine
  VARIANT 5: note = Val or any amino acid with a reactive group in its side chain, such as cysteine
  VARIANT 7: note = Ser or any amino acid with a reactive group in its side chain, such as cysteine
  VARIANT 8: note = Gly or any amino acid with a reactive group in its side chain, such as cysteine
  VARIANT 10: note = Gly or any amino acid with a reactive group in its side chain, such as cysteine
  VARIANT 11: note = Leu, Val Ser, Met, Trp, Phe, Thr, Gln, Glu, Ala, Arg, Gly, Lys, Tyr, Asn, Pro or Ile, preferably Leu or Val, or any other amino acid with a reactive group in its side chain, such as cysteine
  VARIANT 12: note = Val or any amino acid with a reactive group in its side chain, such as cysteine
  VARIANT 13: note = Gln or any amino acid with a reactive group in its side chain, such as cysteine
  VARIANT 14: note = Ala or any amino acid with a reactive group in its side chain, such as cysteine
  VARIANT 15: note = Gly or any amino acid with a reactive group in its side chain, such as cysteine
  VARIANT 17: note = Ser or any amino acid with a reactive group in its side chain, such as cysteine
  VARIANT 18: note = Leu or any amino acid with a reactive group in its side chain, such as cysteine
  VARIANT 19: note = Ser or any amino acid with a reactive group in its side chain, such as cysteine
  VARIANT 21: note = Ser or any amino acid with a reactive group in its side chain, such as cysteine
  VARIANT 23: note = Ala or any amino acid with a reactive group in its side chain, such as cysteine
  VARIANT 25: note = Ser or any amino acid with a reactive group in its side chain, such as cysteine
  VARIANT 26: note = Gly or any amino acid with a reactive group in its side chain, such as cysteine
  VARIANT 27: note = Gly or any amino acid with a reactive group in its side chain, such as cysteine
  VARIANT 28: note = Ser or any amino acid with a reactive group in its side chain, such as cysteine
  VARIANT 30: note = Ser or any amino acid with a reactive group in its side chain, such as cysteine
  VARIANT 31: note = Asn or any amino acid with a reactive group in its side chain, such as cysteine
  VARIANT 32: note = Tyr or any amino acid with a reactive group in its side chain, such as cysteine
  VARIANT 39: note = Gln or any amino acid with a reactive group in its side chain, such as cysteine
  VARIANT 41: note = Pro or any amino acid with a reactive group in its side chain, such as cysteine
  VARIANT 42: note = Gly or any amino acid with a reactive group in its side chain, such as cysteine
  VARIANT 43: note = Lys or any amino acid with a reactive group in its side chain, such as cysteine
  VARIANT 44: note = Glu or any amino acid with a reactive group in its side chain, such as cysteine
  VARIANT 45: note = Arg or any amino acid with a reactive group in its side chain, such as cysteine
  VARIANT 46: note = Glu or any amino acid with a reactive group in its side chain, such as cysteine
  VARIANT 53: note = Trp or any amino acid with a reactive group in its side chain, such as cysteine
  VARIANT 54: note = Arg or any amino acid with a reactive group in its side chain, such as cysteine
  VARIANT 55: note = Gly or any amino acid with a reactive group in its side chain, such as cysteine
  VARIANT 56: note = Asp or any amino acid with a reactive group in its side chain, such as cysteine
  VARIANT 57: note = Ile or any amino acid with a reactive group in its side chain, such as cysteine
  VARIANT 58: note = Thr or any amino acid with a reactive group in its side chain, such as cysteine
  VARIANT 59: note = be Ile or any amino acid with a reactive group in its side chain, such as cysteine
  VARIANT 60: note = Gly or any amino acid with a reactive group in its side chain, such as cysteine
  VARIANT 62: note = Pro or any amino acid with a reactive group in its side chain, such as cysteine
  VARIANT 63: note = Asn or any amino acid with a reactive group in its side chain, such as cysteine
  VARIANT 65: note = Glu or any amino acid with a reactive group in its side chain, such as cysteine
  VARIANT 66: note = Gly or any amino acid with a reactive group in its side chain, such as cysteine
  VARIANT 67: note = Arg or any amino acid with a reactive group in its side chain, such as cysteine
  VARIANT 69: note = Thr or any amino acid with a reactive group in its side chain, such as cysteine
  VARIANT 71: note = Ser or any amino acid with a reactive group in its side chain, such as cysteine
  VARIANT 72: note = Arg or any amino acid with a reactive group in its side chain, such as cysteine
  VARIANT 73: note = Asp or any amino acid with a reactive group in its side chain, such as cysteine
  VARIANT 74: note = Asn or any amino acid with a reactive group in its side chain, such as cysteine
  VARIANT 75: note = Ala or any amino acid with a reactive group in its side chain, such as cysteine
  VARIANT 76: note = Lys or any amino acid with a reactive group in its side chain, such as cysteine
  VARIANT 77: note = Asn or any amino acid with a reactive group in its side chain, such as cysteine
  VARIANT 80: note = Tyr or any amino acid with a reactive group in its side chain, such as cysteine
  VARIANT 82: note = Gln or any amino acid with a reactive group in its side chain, such as cysteine
  VARIANT 84: note = Asn or any amino acid with a reactive group in its side chain, such as cysteine
  VARIANT 85: note = Ser or any amino acid with a reactive group in its side chain, such as cysteine
  VARIANT 87: note = Ala or any amino acid with a reactive group in its side chain, such as cysteine
  VARIANT 88: note = Pro or any amino acid with a reactive group in its side chain, such as cysteine
  VARIANT 89: note = Asp or any amino acid with a reactive group in its side chain, such as cysteine
  VARIANT 91: note = Thr or any amino acid with a reactive group in its side chain, such as cysteine
  VARIANT 93: note = Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val or any other amino acid with a reactive group in its side chain, such as cysteine
  VARIANT 95: note = Tyr or any amino acid with a reactive group in its side chain, such as cysteine
  VARIANT 100: note = Thr or any amino acid with a reactive group in its side chain, such as cysteine
  VARIANT 102: note = Leu or any amino acid with a reactive group in its side chain, such as cysteine
  VARIANT 103: note = Asn or any amino acid with a reactive group in its side chain, such as cysteine
  VARIANT 104: note = Pro or any amino acid with a reactive group in its side chain, such as cysteine
  VARIANT 105: note = Gly or any amino acid with a reactive group in its side chain, such as cysteine
  VARIANT 108: note = Ile or any amino acid with a reactive group in its side chain, such as cysteine
  VARIANT 109: note = Tyr or any amino acid with a reactive group in its side chain, such as cysteine
  VARIANT 110: note = Asp or any amino acid with a reactive group in its side chain, such as cysteine
  VARIANT 111: note = Trp or any amino acid with a reactive group in its side chain, such as cysteine
  VARIANT 114: note = Asp or any amino acid with a reactive group in its side chain, such as cysteine
  VARIANT 115: note = Tyr or any amino acid with a reactive group in its side chain, such as cysteine
  VARIANT 116: note = Trp or any amino acid with a reactive group in its side chain, such as cysteine
  VARIANT 118: note = Arg or any amino acid with a reactive group in its side chain, such as cysteine
  VARIANT 119: note = Gly or any amino acid with a reactive group in its side chain, such as cysteine
  VARIANT 121: note = be Gln, Leu, Arg, Pro, Glu, Lys, Ser, Thr, Met, Ala or His; preferably Gln or Leu, or any other amino acid with a reactive group in its side chain, such as cysteine
  VARIANT 123: note = Thr or any amino acid with a reactive group in its side chain, such as cysteine
  VARIANT 125: note = Ser or any amino acid with a reactive group in its side chain, such as cysteine
  VARIANT 126: note = Ser or any amino acid with a reactive group in its side chain, such as cysteine
  VARIANT 127: note = absent or Gly
  VARIANT 128: note = absent or Gly
  VARIANT 129: note = absent or Cys
XVXLXEXXGXXXXXXGXXXIXCXAXXXXLXXXVLGWFRXAXXXXXXFVAAINXXXXXXXXPXXVXXXFXIXXXXXXXTGXLXMXXLXXXDXAXYXCGAGXPXXXXAYXXXXSYXXXGXXTXVXVXXXXX

SEQ ID NO: 187 - protein, 156 aa, synthetic construct
DLGKKLLEAARAGQDDEVRILMANGADVNAHDTFGFTPLHLAALYGHLEIVEVLLKNGADVNADDSYGRTPLHLAAMRGHLEIVEVLLKYGADVNAADEEGRTPLHLAAKRGHLEIVEVLLKNGADVNAQDKFGKTAFDISIDNGNEDLAEILQKL

SEQ ID NO: 188 - protein, 157 aa, synthetic construct
  VARIANT 1: note = Asp or any amino acid with a reactive group in its side chain, such as cysteine;
  VARIANT 2: note = Leu or any amino acid with a reactive group in its side chain, such as cysteine
  VARIANT 4: note = Lys or any amino acid with a reactive group in its side chain, such as cysteine
  VARIANT 8: note = Glu or any amino acid with a reactive group in its side chain, such as cysteine
  VARIANT 11: note = Arg or any amino acid with a reactive group in its side chain, such as cysteine
  VARIANT 12: note = Ala or any amino acid with a reactive group in its side chain, such as cysteine
  VARIANT 13: note = Gly or any amino acid with a reactive group in its side chain, such as cysteine
  VARIANT 14: note = Gln or any amino acid with a reactive group in its side chain, such as cysteine
  VARIANT 15: note = Asp or any amino acid with a reactive group in its side chain, such as cysteine
  VARIANT 16: note = Asp or any amino acid with a reactive group in its side chain, such as cysteine
  VARIANT 17: note = Glu or any amino acid with a reactive group in its side chain, such as cysteine
  VARIANT 19: note = Arg or any amino acid with a reactive group in its side chain, such as cysteine
  VARIANT 20: note = Ile or any amino acid with a reactive group in its side chain, such as cysteine
  VARIANT 23: note = Ala or any amino acid with a reactive group in its side chain, such as cysteine
  VARIANT 24: note = Asn or any amino acid with a reactive group in its side chain, such as cysteine
  VARIANT 25: note = Gly or any amino acid with a reactive group in its side chain, such as cysteine
  VARIANT 27: note = Asp or any amino acid with a reactive group in its side chain, such as cysteine
  VARIANT 29: note = Asn or any amino acid with a reactive group in its side chain, such as cysteine
  VARIANT 31: note = His or any amino acid with a reactive group in its side chain, such as cysteine
  VARIANT 32: note = Asp or any amino acid with a reactive group in its side chain, such as cysteine
  VARIANT 33: note = Thr or any amino acid with a reactive group in its side chain, such as cysteine
  VARIANT 34: note = Phe or any amino acid with a reactive group in its side chain, such as cysteine
  VARIANT 36: note = Phe or any amino acid with a reactive group in its side chain, such as cysteine
  VARIANT 44: note = Leu or any amino acid with a reactive group in its side chain, such as cysteine
  VARIANT 45: note = Tyr or any amino acid with a reactive group in its side chain, such as cysteine
  VARIANT 46: note = Gly or any amino acid with a reactive group in its side chain, such as cysteine
  VARIANT 47: note = His or any amino acid with a reactive group in its side chain, such as cysteine
  VARIANT 48: note = Leu or any amino acid with a reactive group in its side chain, such as cysteine
  VARIANT 49: note = Glu or any amino acid with a reactive group in its side chain, such as cysteine
  VARIANT 52: note = Glu or any amino acid with a reactive group in its side chain, such as cysteine
  VARIANT 56: note = Lys or any amino acid with a reactive group in its side chain, such as cysteine
  VARIANT 57: note = Asn or any amino acid with a reactive group in its side chain, such as cysteine
  VARIANT 58: note = Gly or any amino acid with a reactive group in its side chain, such as cysteine
  VARIANT 60: note = Asp or any amino acid with a reactive group in its side chain, such as cysteine
  VARIANT 62: note = Asn or any amino acid with a reactive group in its side chain, such as cysteine
  VARIANT 64: note = Asp or any amino acid with a reactive group in its side chain, such as cysteine
  VARIANT 66: note = Ser or any amino acid with a reactive group in its side chain, such as cysteine
  VARIANT 67: note = Tyr or any amino acid with a reactive group in its side chain, such as cysteine
  VARIANT 77: note = Met or any amino acid with a reactive group in its side chain, such as cysteine
  VARIANT 78: note = Arg or any amino acid with a reactive group in its side chain, such as cysteine
  VARIANT 79: note = Gly or any amino acid with a reactive group in its side chain, such as cysteine
  VARIANT 80: note = His or any amino acid with a reactive group in its side chain, such as cysteine
  VARIANT 81: note = Leu or any amino acid with a reactive group in its side chain, such as cysteine
  VARIANT 82: note = Glu or any amino acid with a reactive group in its side chain, such as cysteine
  VARIANT 85: note = Glu or any amino acid with a reactive group in its side chain, such as cysteine
  VARIANT 89: note = Lys or any amino acid with a reactive group in its side chain, such as cysteine
  VARIANT 90: note = Tyr or any amino acid with a reactive group in its side chain, such as cysteine
  VARIANT 91: note = Gly or any amino acid with a reactive group in its side chain, such as cysteine
  VARIANT 93: note = Asp or any amino acid with a reactive group in its side chain, such as cysteine
  VARIANT 95: note = Asn or any amino acid with a reactive group in its side chain, such as cysteine
  VARIANT 97: note = Ala or any amino acid with a reactive group in its side chain, such as cysteine
  VARIANT 99: note = Glu or any amino acid with a reactive group in its side chain, such as cysteine
  VARIANT 100: note = Glu or any amino acid with a reactive group in its side chain, such as cysteine
  VARIANT 107: note = Leu or any amino acid with a reactive group in its side chain, such as cysteine
  VARIANT 110: note = Lys or any amino acid with a reactive group in its side chain, such as cysteine
  VARIANT 111: note = Ala or any amino acid with a reactive group in its side chain, such as cysteine
  VARIANT 112: note = Gly or any amino acid with a reactive group in its side chain, such as cysteine
  VARIANT 113: note = His or any amino acid with a reactive group in its side chain, such as cysteine
  VARIANT 114: note = Leu or any amino acid with a reactive group in its side chain, such as cysteine
  VARIANT 115: note = Glu or any amino acid with a reactive group in its side chain, such as cysteine
  VARIANT 118: note = Glu or any amino acid with a reactive group in its side chain, such as cysteine
  VARIANT 119: note = Val or any amino acid with a reactive group in its side chain, such as cysteine
  VARIANT 121: note = Leu or any amino acid with a reactive group in its side chain, such as cysteine
  VARIANT 122: note = Lys or any amino acid with a reactive group in its side chain, such as cysteine
  VARIANT 123: note = Asn or any amino acid with a reactive group in its side chain, such as cysteine
  VARIANT 124: note = Gly or any amino acid with a reactive group in its side chain, such as cysteine
  VARIANT 126: note = Asp or any amino acid with a reactive group in its side chain, such as cysteine
  VARIANT 127: note = Val or any amino acid with a reactive group in its side chain, such as cysteine
  VARIANT 128: note = Asn or any amino acid with a reactive group in its side chain, such as cysteine
  VARIANT 130: note = Gln or any amino acid with a reactive group in its side chain, such as cysteine
  VARIANT 132: note = Lys or any amino acid with a reactive group in its side chain, such as cysteine
  VARIANT 133: note = Phe or any amino acid with a reactive group in its side chain, such as cysteine
  VARIANT 134: note = Gly or any amino acid with a reactive group in its side chain, such as cysteine
  VARIANT 135: note = Lys or any amino acid with a reactive group in its side chain, such as cysteine
  VARIANT 138: note = Phe or any amino acid with a reactive group in its side chain, such as cysteine
  VARIANT 139: note = Asp or any amino acid with a reactive group in its side chain, such as cysteine
  VARIANT 142: note = Ile or any amino acid with a reactive group in its side chain, such as cysteine
  VARIANT 143: note = Asp or any amino acid with a reactive group in its side chain, such as cysteine
  VARIANT 144: note = Asn or any amino acid with a reactive group in its side chain, such as cysteine
  VARIANT 145: note = Gly or any amino acid with a reactive group in its side chain, such as cysteine
  VARIANT 146: note = Asn or any amino acid with a reactive group in its side chain, such as cysteine
  VARIANT 147: note = Glu or any amino acid with a reactive group in its side chain, such as cysteine
  VARIANT 148: note = Asp or any amino acid with a reactive group in its side chain, such as cysteine
  VARIANT 151: note = Glu or any amino acid with a reactive group in its side chain, such as cysteine
  VARIANT 152: note = Ile or any amino acid with a reactive group in its side chain, such as cysteine
  VARIANT 154: note = Gln or any amino acid with a reactive group in its side chain, such as cysteine
  VARIANT 155: note = Lys or any amino acid with a reactive group in its side chain, such as cysteine
  VARIANT 156: note = absent or Leu
  VARIANT 157: note = absent or Cys
XXGXXLLXAAXXXXXXXVXXLMXXXAXVXAXXXXGXTPLHLAAXXXXXXIVXVLLXXXAXVXAXDXXGATPLHLAAXXXXXXIVXVLLXXXAXVXAXDXXGATPLHXAAXXXXXXIVXXLXXXXAXXXAXDXXXXTAXXISXXXXXXXLAXXLXXXX

SEQ ID NO: 189 - protein, 157 aa, synthetic construct
  VARIANT 5: note = Lys or any amino acid with a reactive group in its side chain, such as cysteine
  VARIANT 49: note = Glu or any amino acid with a reactive group in its side chain, such as cysteine
  VARIANT 60: note = Asp or any amino acid with a reactive group in its side chain, such as cysteine
  VARIANT 64: note = Asp or any amino acid with a reactive group in its side chain, such as cysteine
  VARIANT 82: note = Glu or any amino acid with a reactive group in its side chain, such as cysteine
  VARIANT 93: note = Asp or any amino acid with a reactive group in its side chain, such as cysteine
  VARIANT 97: note = Ala or any amino acid with a reactive group in its side chain, such as cysteine
  VARIANT 100: note = Glu or any amino acid with a reactive group in its side chain, such as cysteine
  VARIANT 115: note = Glu or any amino acid with a reactive group in its side chain, such as cysteine
  VARIANT 126: note = Asp or any amino acid with a reactive group in its side chain, such as cysteine
  VARIANT 148: note = Asp or any amino acid with a reactive group in its side chain, such as cysteine
  VARIANT 155: note = Lys or any amino acid with a reactive group in its side chain, such as cysteine
  VARIANT 85: note = Glu or any amino acid with a reactive group in its side chain, such as cysteine
  VARIANT 95: note = Asn or any amino acid with a reactive group in its side chain, such as cysteine
  VARIANT 143: note = Asp or any amino acid with a reactive group in its side chain, such as cysteine
  VARIANT 156: note = absent or Leu
  VARIANT 157: note = absent or Cys
DLGKXLLEAARAGQDDEVRILMANGADVNAHDTFGFTPLHLAALYGHLXIVEVLLKNGAXVNAXDSYGATPLHLAAMRGHLXIVXVLLKYGAXVXAXDEXGATPLHLAAKAGHLXIVEVLLKNGAXVNAQDKFGKTAFDISIXNGNEXLAEILQXXX

SEQ ID NO: 190 - protein, 78 aa, synthetic construct
SHKQIYYSDKYDDEEFEYRHVMLPKDIAKLVPKTHLMSESEWRNLGVQQSQGWVHYMIHEPEPHILLFRRPLPKKPKK

SEQ ID NO: 191 - protein, 78 aa, synthetic construct
  VARIANT 1: note = Ser or any amino acid with a reactive group in its side chain, such as cysteine
  VARIANT 2: note = His or any amino acid with a reactive group in its side chain, such as cysteine
  VARIANT 3: note = Lys or any amino acid with a reactive group in its side chain, such as cysteine
  VARIANT 4: note = Gln or any amino acid with a reactive group in its side chain, such as cysteine
  VARIANT 6: note = Tyr or any amino acid with a reactive group in its side chain, such as cysteine
  VARIANT 7: note = Tyr or any amino acid with a reactive group in its side chain, such as cysteine
  VARIANT 9: note = Asp or any amino acid with a reactive group in its side chain, such as cysteine
  VARIANT 10: note = Lys or any amino acid with a reactive group in its side chain, such as cysteine
  VARIANT 11: note = Tyr or any amino acid with a reactive group in its side chain, such as cysteine
  VARIANT 12: note = Tyr or any amino acid with a reactive group in its side chain, such as cysteine
  VARIANT 13: note = Asp or any amino acid with a reactive group in its side chain, such as cysteine
  VARIANT 14: note = Glu or any amino acid with a reactive group in its side chain, such as cysteine
  VARIANT 15: note = Glu or any amino acid with a reactive group in its side chain, such as cysteine
  VARIANT 16: note = Phe or any amino acid with a reactive group in its side chain, such as cysteine
  VARIANT 17: note = Glu or any amino acid with a reactive group in its side chain, such as cysteine
  VARIANT 18: note = Tyr or any amino acid with a reactive group in its side chain, such as cysteine
  VARIANT 19: note = Arg or any amino acid with a reactive group in its side chain, such as cysteine
  VARIANT 20: note = His or any amino acid with a reactive group in its side chain, such as cysteine
  VARIANT 22: note = Met or any amino acid with a reactive group in its side chain, such as cysteine
  VARIANT 25: note = Lys or any amino acid with a reactive group in its side chain, such as cysteine
  VARIANT 26: note = Asp or any amino acid with a reactive group in its side chain, such as cysteine
  VARIANT 27: note = Ile or any amino acid with a reactive group in its side chain, such as cysteine
  VARIANT 29: note = Lys or any amino acid with a reactive group in its side chain, such as cysteine
  VARIANT 30: note = Leu or any amino acid with a reactive group in its side chain, such as cysteine
  VARIANT 32: note = Pro or any amino acid with a reactive group in its side chain, such as cysteine
  VARIANT 33: note = Lys or any amino acid with a reactive group in its side chain, such as cysteine
  VARIANT 34: note = Thr or any amino acid with a reactive group in its side chain, such as cysteine
  VARIANT 35: note = His or any amino acid with a reactive group in its side chain, such as cysteine
  VARIANT 36: note = Leu or any amino acid with a reactive group in its side chain, such as cysteine
  VARIANT 38: note = Ser or any amino acid with a reactive group in its side chain, such as cysteine
  VARIANT 39: note = Glu or any amino acid with a reactive group in its side chain, such as cysteine
  VARIANT 40: note = Ser or any amino acid with a reactive group in its side chain, such as cysteine
  VARIANT 41: note = Glu or any amino acid with a reactive group in its side chain, such as cysteine
  VARIANT 43: note = Arg or any amino acid with a reactive group in its side chain, such as cysteine
  VARIANT 44: note = Asn or any amino acid with a reactive group in its side chain, such as cysteine
  VARIANT 46: note = Gly or any amino acid with a reactive group in its side chain, such as cysteine
  VARIANT 48: note = Gln or any amino acid with a reactive group in its side chain, such as cysteine
  VARIANT 50: note = Ser or any amino acid with a reactive group in its side chain, such as cysteine
  VARIANT 51: note = Gln or any amino acid with a reactive group in its side chain, such as cysteine
  VARIANT 52: note = Gly or any amino acid with a reactive group in its side chain, such as cysteine
  VARIANT 54: note = Val or any amino acid with a reactive group in its side chain, such as cysteine
  VARIANT 56: note = Tyr or any amino acid with a reactive group in its side chain, such as cysteine
  VARIANT 57: note = Met or any amino acid with a reactive group in its side chain, such as cysteine
  VARIANT 58: note = Ile or any amino acid with a reactive group in its side chain, such as cysteine
  VARIANT 59: note = His or any amino acid with a reactive group in its side chain, such as cysteine
  VARIANT 60: note = Glu or any amino acid with a reactive group in its side chain, such as cysteine
  VARIANT 61: note = Pro or any amino acid with a reactive group in its side chain, such as cysteine
  VARIANT 62: note = Glu or any amino acid with a reactive group in its side chain, such as cysteine
  VARIANT 63: note = Pro or any amino acid with a reactive group in its side chain, such as cysteine
  VARIANT 64: note = His or any amino acid with a reactive group in its side chain, such as cysteine
  VARIANT 69: note = Arg or any amino acid with a reactive group in its side chain, such as cysteine
  VARIANT 70: note = Arg or any amino acid with a reactive group in its side chain, such as cysteine
  VARIANT 71: note = Pro or any amino acid with a reactive group in its side chain, such as cysteine
  VARIANT 72: note = Leu or any amino acid with a reactive group in its side chain, such as cysteine
  VARIANT 73: note = Pro or any amino acid with a reactive group in its side chain, such as cysteine
  VARIANT 74: note = Lys or any amino acid with a reactive group in its side chain, such as cysteine
  VARIANT 75: note = Lys or any amino acid with a reactive group in its side chain, such as cysteine
  VARIANT 76: note = Pro or any amino acid with a reactive group in its side chain, such as cysteine
  VARIANT 77: note = Lys or any amino acid with a reactive group in its side chain, such as cysteine
  VARIANT 78: note = Lys or any amino acid with a reactive group in its side chain, such as cysteine
XXXXIXXSXXXXXXXXXXXXVXLPXXXAXXVXXXXXMXXXXWXXLXVXQXXXWXHXXXXXXXXXILLFXXXXXXXXXX

SEQ ID NO: 192 - protein, 78 aa, synthetic construct
  VARIANT 1: note = Ser or any amino acid with a reactive group in its side chain, such as cysteine
  VARIANT 4: note = Gln or any amino acid with a reactive group in its side chain, such as cysteine
  VARIANT 10: note = Lys or any amino acid with a reactive group in its side chain, such as cysteine
  VARIANT 12: note = Asp or any amino acid with a reactive group in its side chain, such as cysteine
  VARIANT 25: note = Lys or any amino acid with a reactive group in its side chain, such as cysteine
  VARIANT 29: note = Lys or any amino acid with a reactive group in its side chain, such as cysteine
  VARIANT 33: note = Lys or any amino acid with a reactive group in its side chain, such as cysteine
XHKXIYYSDXYXDEEFEYRHVMLPXDIAXLVPXTHLMSESEWRNLGVQQSQGWVHYMIHEPEPHILLFRRPLPKKPKK

SEQ ID NO: 193 - protein, 15 aa, synthetic construct
EPKSCDKTHTCPPCP

SEQ ID NO: 194 - protein, 24 aa, synthetic construct
GGGGSGGGSEPKSCDKTHTCPPCP

SEQ ID NO: 195 - protein, 12 aa, synthetic construct
EPKTPKPQPAAA

SEQ ID NO: 196 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 197 - protein, 157 aa, synthetic construct
DLGKKLLEAARAGQDDEVRILMANGADVNAHDTFGFTPLHLAALYGHLEIVEVLLKNGADVNADDSYGRTPLHLAAMRGHLEIVEVLLKYGADVNAADEEGRTPLHLAAKRGHLEIVEVLLKNGADVNAQDKFGKTAFDISIDNGNEDLAEILQKLC

SEQ ID NO: 198 - protein, 287 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSDLGKKLLEAARAGQDDEVRILMANGADVNAHDTFGFTPLHLAALYGHLEIVEVLLKNGADVNADDSYGRTPLHLAAMRGHLEIVEVLLKYGADVNAADEEGRTPLHLAAKRGHLEIVEVLLKNGADVNAQDKFGKTAFDISIDNGNEDLAEILQKLC

SEQ ID NO: 199 - protein, 156 aa, synthetic construct
DLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISIDNGNEDLAEILQKC

SEQ ID NO: 200 - protein, 286 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSDLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISIDNGNEDLAEILQKC

SEQ ID NO: 201 - protein, 78 aa, synthetic construct
SHKQIYYSDKCDDEEFEYRHVMLPKDIAKLVPKTHLMSESEWRNLGVQQSQGWVHYMIHEPEPHILLFRRPLPKKPKK

SEQ ID NO: 202 - protein, 73 aa, synthetic construct
SHKQIYYSDKYDCEEFEYRHVMLPKDIAKLVPKTHLMSESEWRNLGVQQSQGWVHYMIHEPEPHILLFRRPLP

SEQ ID NO: 203 - protein, 208 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDKCDDEEFEYRHVMLPKDIAKLVPKTHLMSESEWRNLGVQQSQGWVHYMIHEPEPHILLFRRPLPKKPKK

SEQ ID NO: 204 - protein, 203 aa, synthetic construct
DVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGGSGGGGSSHKQIYYSDKYDCEEFEYRHVMLPKDIAKLVPKTHLMSESEWRNLGVQQSQGWVHYMIHEPEPHILLFRRPLP

SEQ ID NO: 205 - protein, 78 aa, synthetic construct
  VARIANT 9: note = Asp or any amino acid with a reactive group in its side chain, such as cysteine
  VARIANT 10: note = Lys or any amino acid with a reactive group in its side chain, such as cysteine
  VARIANT 11: note = Tyr or any amino acid with a reactive group in its side chain, such as cysteine
  VARIANT 12: note = Asp or any amino acid with a reactive group in its side chain, such as cysteine
  VARIANT 13: note = Asp or any amino acid with a reactive group in its side chain, such as cysteine
  VARIANT 22: note = Met or any amino acid with a reactive group in its side chain, such as cysteine
  VARIANT 33: note = Lys or any amino acid with a reactive group in its side chain, such as cysteine
  VARIANT 51: note = Gln or any amino acid with a reactive group in its side chain, such as cysteine
  VARIANT 57: note = Met or any amino acid with a reactive group in its side chain, such as cysteine
  VARIANT 78: note = Lys or any amino acid with a reactive group in its side chain, such as cysteine
SHKQIYYSXXXXXEEFEYRHVXLPKDIAKLVPXTHLMSESEWRNLGVQQSXGWVHYXIHEPEPHILLFRRPLPKKPKX

SEQ ID NO: 206 - protein, 129 aa, synthetic construct
  VARIANT 1: note = Glu or any amino acid with a reactive group in its side chain, such as cysteine
  VARIANT 7: note = Ser or any amino acid with a reactive group in its side chain, such as cysteine
  VARIANT 11: note = Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val
  VARIANT 13: note = Gln or any amino acid with a reactive group in its side chain, such as cysteine
  VARIANT 17: note = Ser or any amino acid with a reactive group in its side chain, such as cysteine
  VARIANT 19: note = Ser or any amino acid with a reactive group in its side chain, such as cysteine
  VARIANT 21: note = Ser or any amino acid with a reactive group in its side chain, such as cysteine
  VARIANT 23: note = Ala or any amino acid with a reactive group in its side chain, such as cysteine
  VARIANT 25: note = Ser or any amino acid with a reactive group in its side chain, such as cysteine
  VARIANT 26: note = Gly or any amino acid with a reactive group in its side chain, such as cysteine
  VARIANT 28: note = Ser or any amino acid with a reactive group in its side chain, such as cysteine
  VARIANT 31: note = Asn or any amino acid with a reactive group in its side chain, such as cysteine
  VARIANT 43: note = Lys or any amino acid with a reactive group in its side chain, such as cysteine
  VARIANT 44: note = Glu or any amino acid with a reactive group in its side chain, such as cysteine
  VARIANT 56: note = Asp or any amino acid with a reactive group in its side chain, such as cysteine
  VARIANT 63: note = Asn or any amino acid with a reactive group in its side chain, such as cysteine
  VARIANT 66: note = Gly or any amino acid with a reactive group in its side chain, such as cysteine
  VARIANT 69: note = Thr or any amino acid with a reactive group in its side chain, such as cysteine
  VARIANT 71: note = Ser or any amino acid with a reactive group in its side chain, such as cysteine
  VARIANT 73: note = Asp or any amino acid with a reactive group in its side chain, such as cysteine
  VARIANT 75: note = Ala or any amino acid with a reactive group in its side chain, such as cysteine
  VARIANT 76: note = Lys or any amino acid with a reactive group in its side chain, such as cysteine
  VARIANT 85: note = Ser or any amino acid with a reactive group in its side chain, such as cysteine
  VARIANT 89: note = Asp or any amino acid with a reactive group in its side chain, such as cysteine
  VARIANT 93: note = Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val
  VARIANT 105: note = Gly or any amino acid with a reactive group in its side chain, such as cysteine
  VARIANT 110: note = Asp or any amino acid with a reactive group in its side chain, such as cysteine
  VARIANT 118: note = Arg or any amino acid with a reactive group in its side chain, such as cysteine
  VARIANT 121: note = Gln, Leu, Arg, Pro, Glu, Lys, Ser, Thr, Met, Ala or His; preferably Gln or Leu
  VARIANT 125: note = Ser or any amino acid with a reactive group in its side chain, such as cysteine
  VARIANT 127: note = absent or Gly
  VARIANT 128: note = absent or Gly
  VARIANT 129: note = absent or Cys
XVQLVEXGGGZVXAGGXLXIXCXAXXGXLSXYVLGWFRQAPGXXREFVAAINWRGXITIGPPXVEXRFXIXRXNXXNTGYLQMNXLAPXDTAZYYCGAGTPLNPXAYIYXWSYDYWGXGTZVTVXSXXX

SEQ ID NO: 207 - protein, 564 aa, synthetic construct
DVQLVESGGGSVQAGDSLRLSCAASGRTFGSIRVGWFRQTPGKEREFVAAINRNDGTTYYADSVKGRFTISRDNAKNTVYMQMASLKPEDTAVYYCAAGLQYNRSADRVPVGAVYWGQGTLVTVSSGGGGSGGGSEVQLVESGGGSVQAGDSLRLSCAASGRTFGSIRVGWFRQTPGKEREFVAAINRNDGTTYYADSVKGRFTISRDNAKNTVYMQMASLKPEDTAVYYCAAGLQYNRSADRVPVGAVYWGQGTLVTVSSGGGGSGGGGSGGGGSGGGGSGGGGSGGGGSGGGGSEVQLVESGGGLVQPGGSLRLSCAASGLTFSTNPMYWYRQAPGKQRELVASISSRGITNYADSVKGRFTISRDNSKNTVYLQMNSLRPEDTAVYYCRLASLSSGTVYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 208 - protein, 157 aa, synthetic construct
DLGKKLLEAARAGQDDEVRILMANGADVNAHDTFGFTPLHLAALYGHLEIVEVLLKNGADVNADDSYGATPLHLAAMRGHLEIVEVLLKYGADVNAADEEGATPLHLAAKAGHLEIVEVLLKNGADVNAQDKFGKTAFDISIDNGNEDLAEILQKLC

SEQ ID NO: 209 - protein, 5 aa, synthetic construct
  VARIANT 3: note = any amino acid
LPXTG

SEQ ID NO: 210 - protein, 6 aa, synthetic construct
  VARIANT 3: note = any residue
LPXTGG

SEQ ID NO: 211 - protein, 5 aa, synthetic construct
  VARIANT 3: note = any amino acid
LPXTA

SEQ ID NO: 212 - protein, 12 aa, synthetic construct
YHWYGYTPQNVI

SEQ ID NO: 213 - protein, 5 aa, synthetic construct
GGSGG

SEQ ID NO: 214 - protein, 120 aa, synthetic construct
EVQLQASGGGLAQPGGSLRLSVTVSGSIDVINNMAWYRQAPGNARELVATITSGFSTNYASSVKGRFTISRDNAKKAVYLQMNSLKPEDTADYYSKVHLIRLGAARAYDYWGQGTQVTVS

SEQ ID NO: 215 - protein, 228 aa, synthetic construct
DVQLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSHKQIYYSDKCDDEEFEYRHVMLPKDIAKLVPKTHLMSESEWRNLGVQQSCGWVHYMIHEPEPHILLFRRPLPKKPKCGGGPAAKRVKLD

SEQ ID NO: 216 - protein, 124 aa, synthetic construct
DVQLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 217 - protein, 12 aa, synthetic construct
GGGPAAKRVKLD

SEQ ID NO: 218 - protein, 28 aa, synthetic construct
GGGIGAVLEVLTTGLPALISWIEEEEQQ

SEQ ID NO: 219 - protein, 17 aa, synthetic construct
  MOD_RES 17: note = Amidated
GGSGGYHWYGYTPQNVI

SEQ ID NO: 220 - protein, 28 aa, synthetic construct
  MOD_RES 28: note = Amidated
GGGIGAVLEVLTTGLPALISWIEEEEQQ

SEQ ID NO: 221 - protein, 9 aa, synthetic construct
PAAKRVKLD

SEQ ID NO: 222 - protein, 126 aa, synthetic construct
EVQLVESGGGLVQAGGSLSICCAASGGSLSCYVLGWFRQAPGCEREFVAAINWRGDITIGPPNVEGRFCISRCNAKNTGYLQMNCLAPDDTAVYYCGAGTPLNPCAYIYCWSYDYWGCGTLVTVSS

SEQ ID NO: 223 - protein, 126 aa, synthetic construct
EVQLVECGGGLVCAGGSLCISCCASGGSLSNYVLGWFRQAPGKCREFVAAINWRGCITIGPPCVEGRFTICRDNCKNTGYLQMNSLAPDDTAVYYCGAGTPLNPGAYIYDWSYDYWGRGTLVTVSS

SEQ ID NO: 224 - protein, 126 aa, synthetic construct
EVQLVECGGGLVQAGGCLSISCAASGGSLSCYVLGWFRQAPGKCREFVAAINWRGCITIGPPCVEGRFCISRDNACNTGYLQMNSLAPDDTAVYYCGAGTPLNPGAYIYDWSYDYWGRGTLVTVCS

(19) United States
(12) Patent Application Publication
(10) Pub. No.: US 2024/0415974 A1
(43) Pub. Date: Dec. 19, 2024

(54) PROTEIN-BASED CONJUGATION CARRIERS

(71) Applicant: Ablynx N.V., Zwijnaarde (BE)

(72) Inventors: Carlo Boutton, Wielsbeke (BE); Peter Casteels, Erpe-Mere (BE); Alexandra Ferrier Berthelot, Paris (FR); Raf Ponsaerts, Kampenhout (BE); John Reed, Cambridge, MA (US)

(73) Assignee: Ablynx N.V., Zwijnaarde (BE)

(21) Appl. No.: 18/394,101

(22) Filed: Dec. 22, 2023

Related U.S. Application Data
(60) Provisional application No. 63/476,994, filed on Dec. 23, 2022.

(30) Foreign Application Priority Data
Jan. 9, 2023 (EP) 23 150 688.2

(51) Int. Cl. A61K 47/68; A61K 47/62; C07K 16/00; C12N 9/12

(52) U.S. Cl. CPC A61K 47/6801; A61K 47/62; C07K 16/00; C12N 9/12; C07K 2317/22; C07K 2317/24; C07K 2317/569; C07K 2317/92; C07K 2317/94; C07K 2318/20; C12Y 207/11022

(57) ABSTRACT

The present technology relates to the field of drug delivery and provides molecules comprising or consisting of at least one protein-based carrier building block, wherein the protein-based carrier building block comprises at least one, preferably at least two, attachment point(s) or conjugation site(s).

In particular, the technology provides a molecule comprising at least one protein-based building block, wherein the at least one protein-based building block:
a) comprises at least one conjugation site or attachment point;
b) has a molecular mass of 2.5 to 70 kDa;
c) has a globular three-dimensional (3D) structure;
d) has a solubility of 10 mg/ml or more, measured in an aqueous solution at room temperature; and
   does not specifically bind to any human protein or binds one or more human proteins with a K_{D} value greater than 5×10⁻⁴ mol/litre.

### CROSS-REFERENCE TO RELATED APPLICATIONS

This application claims the benefit under 35 U.S.C. § 119 (e) of U.S. provisional application No. 63/476,994, filed Dec. 23, 2022, the entire contents of which are incorporated herein by reference.

### REFERENCE TO AN ELECTRONIC SEQUENCE LISTING

The contents of the electronic sequence listing (A084870224US01-SEQ-JRV.xml; Size: 361,527 bytes; and Date of Creation: Dec. 22, 2023) is herein incorporated by reference in its entirety.

### FIELD OF THE TECHNOLOGY

The present technology provides molecules comprising or consisting of at least one protein-based carrier building block, wherein the protein-based carrier building block comprises at least one, preferably at least two attachment point(s) or conjugation site(s).

The present technology further relates to nucleic acids encoding such molecules or part of such molecules; to host cells comprising such nucleic acids and/or expressing or capable of expressing such molecules or part of such molecules; to compositions, and in particular to pharmaceutical compositions that comprise such molecules, nucleic acids and/or host cells; and to uses of such molecules, nucleic acids, host cells and/or compositions, in particular for labelling, prophylactic, therapeutic and/or diagnostic purposes.

### TECHNOLOGICAL BACKGROUND

Protein-based therapeutics (referred to as “Biologicals”) are creating new therapeutic strategies which are hard to achieve via the classic, small molecule-based therapeutics. One fast-growing field comprises conjugation-based therapeutics such as antibody-drug conjugates (ADCs). For instance, Fatima S W. and Khare S K. (“Benefits and challenges of antibody drug conjugates as novel form of chemotherapy”, J Control Release, 2022, 341:555-565) review recent clinical advances of each component of ADCs (antibody/linker/payload) and how the individual component influences the activity of ADCs. This is also shown, e.g., in Rader, C., “Chemically programmed antibodies”, Trends in Biotechnology, 2014, 32 (4). Antibody-drug conjugates typically rely on the high specificity and affinity of the antibodies, their extended circulatory half-life as well as other effector functions (e.g., complement-dependent cytotoxicity (CDC), antibody-dependent cellular cytotoxicity (ADCC) and antibody-dependent cellular phagocytosis (ADCP). Hence, antibody-drug conjugates and related molecules, such as chemically programmed antibodies, are chosen because of at least one of these functionalities (their targeting properties, half-life extension properties and/or other effector functions, as described above).

Currently, the manufacture of biologicals mainly relies on recombinant production and is hence restricted to polypeptide production which can combine naturally into one functional unit (i.e., IgGs). Typical conjugation strategies are based on site specific cysteine or stochastic lysine conjugations (see, e.g., Zhou et al., 2021, Pharmaceuticals, 14:672; Sadiki et al., 2020, Antibody Therapeutics, 3:271). The rise of non-canonical or novel Amino Acids (nAAs) further add options for conjugation methodology (see, e.g., Malyshev and Romesberg, 2015, Angew Chem Int Ed Engl., 54 (41); Zhang et al., 2017, PNAS, 114 1317).

There is a need of further therapeutic strategies which allow for versatile production and cargo conjugation.

### SUMMARY OF THE TECHNOLOGY

The current technology aims at simplifying the generation of conjugation-based therapeutics and/or to create a plug- and play strategy that can create alternative formats which allow for versatile conjugation of different cargos.

Whereas classic conjugation strategies need to focus on preserving the functionality of the involved polypeptides, a described above, the present technology employs a non-targeting protein-based carrier building block which solely serves as a site-specific conjugation vehicle (protein-based carrier building block). This protein-based carrier building block can be contained within a genetic construct, thus be the product of one manufacturing campaign or, alternatively, can be produced separately (e.g., recombinantly or by alternative means such as solid-phase peptide synthesis, SPPS) and later connected to the active and/or targeting moiety (i.e., the cargo), as it will be explained in detail below. The latter strategy renders more freedom for cargo conjugation conditions onto the protein-based carrier building block. Such freedom can translate into making use of site-specific conjugation onto common amino acids which are usually used in a stochastic way (e.g., lysines) or into conjugation conditions which might impair the functionality/quality of the targeting building block(s) or into alternative production platforms (e.g., chemical synthesis). The position and number of the conjugation sites or attachment points can be engineered/tuned to the specific application.

Hence, the present technology provides molecules comprising or consisting of at least one protein-based carrier building block, and to the protein-based carrier building block as such, as described below, wherein the protein-based carrier building block comprises at least one attachment point or conjugation site, preferably at least two attachment points or conjugation sites. The at least one conjugation site or attachment point is suitable for conjugation or attachment of cargos to the protein-based carrier building block. A “cargo” is any molecule which is/may be attached or conjugated to the protein-based carrier building block through the attachment point(s) or conjugation site(s) present therein. For instance, cargos which may be attached or conjugated to the protein-based carrier building block of the present technology are proteins, peptides, polyethylene glycol (PEG), small molecules, glycans, lipids, chelators, fluorophores, radio isotopes, vitamins such as folic acid or biotin, nucleic acids such as oligonucleotides or siRNA, etc. Hence, in a further embodiment, the present technology provides the protein-based carrier building block attached to at least one cargo, as described herein, i.e., the molecule of the present technology comprises (or, alternatively, consists of) at least one protein-based building block and at least one cargo attached or conjugated to it through the at least one conjugation site or attachment point.

The protein-based carrier building block of the present technology comprises (and, preferably, consists of) at least part of a protein, preferably a whole protein. Hence, preferably, the protein-based carrier building block is a polypeptide. The protein-based carrier building block has a globular 3D structure and is soluble. In addition, the protein-based carrier building block comprised in the molecule of the present technology has a size (molecular mass or molecular weight, MW) of about 2.5 to about 70 kDa, preferably of about 2.5 to less than 50 kDa, more preferably of about 2.5 to about 30 kDa, even more preferably of about 2.5 to about 16 kDa, such as about 6 kDa, or about 7 kDa, or about 16 kDa.

Finally, the protein-based carrier building block of the present technology does not specifically bind to any human protein, although it may show non-specific binding to one or more human proteins, as explained in detail herein. In this case, the protein-based carrier building block may bind to human proteins with low specificity and/or low selectivity, as defined herein. Preferably, the protein-based carrier building block does also not specifically bind to any non-protein (preferably human) molecule, such as DNA, RNA, lipids (e.g., such as phosphatidylserine (PS)) or glycans. The protein-based carrier building block may derive from a target-binding protein (such as an immunoglobulin single variable domain (ISVD), a DARPin, an affibody or an affitin), as described below. It may also derive from other proteins which show specific binding towards, e.g., human proteins, such as small globular human proteins. This is the so-called “protein-based carrier building block precursor”. In these cases, preferably, the protein-based building block does also not specifically bind to any molecule (including non-human proteins) to which the protein-based carrier building block precursor specifically binds (if any). For example, if the precursor of the protein-based carrier building block is an anti-RSV (respiratory syncytial virus) ISVD, the protein-based carrier building block preferably does not specifically bind RSV. Hence, preferably, the protein-based carrier building block does also not specifically bind to the precursor's target, should the precursor have a target and should this be a non-human molecule, such as a non-human protein, or a human non-protein molecule, such as human DNA, RNA, glycans, lipids, etc. In a further preferred embodiment, the protein-based carrier building block does not specifically bind any human protein, non-human protein and/or non-protein molecule when a cargo is conjugated to the at least one, preferably at least two, attachment points or conjugation sites on the protein-based carrier building block.

Hence, the at least one protein-based carrier building block of the present technology:
a) Has at least one attachment point (also referred to as conjugation site in the present description), preferably at least two attachment points or conjugation sites, wherein an attachment point or conjugation site is a reactive group in the side chain of a non-natural or natural amino acid (e.g., Cys, Lys, Tyr, Orn, etc.) preferably located at a solvent-accessible position in the protein-based carrier building block, and/or the N-terminal primary amine, and/or the C-terminal carboxylic group of the protein-based carrier building block, if these are available. The at least one, preferably at least two, attachment point(s) or conjugation site(s) is (are) thus preferably located at solvent-accessible positions in the protein-based carrier building block;
b) Has a size (molecular mass) of from about 2.5 to about 70 kDa, preferably from about 2.5 to about 50 kDa, such as from about 2.5 to less than 50 kDa, more preferably from about 2.5 to about 30 kDa, even more preferably from about 2.5 to about 16 kDa;
c) Has a solubility of about 10 mg/ml or more, measured in an aqueous solution at room temperature (RT), preferably measured in a buffer or water at RT, more preferably measured in a buffer such as citrate buffer or phosphate-buffered saline (PBS) at pH 7.0 or 7.4, at RT, or histidine buffer at pH 6.5, at RT (comprising histidine (10 mM to 100 mM, such as 10 mM), sucrose (1% to 10%, such as 10%) and, optionally, Tween 80 (0.001% to 1%, such as 0.01%)), or phosphate buffer pH 7.0, at RT (comprising NaH₂PO₄/Na₂HPO₄ (10 and 50 mM, such as 10 mM), sodium chloride (NaCl) (100-150 mM, such as 130 mM NaCl) and, optionally, Tween 80 (0.001% to 1%, such as 0.01%)), preferably wherein the buffer is citrate buffer 5 mM or PBS, at pH 7.0 or 7.4;
d) Has a globular 3D structure, as described below;
e) Does not specifically bind to any human protein (or binds one or more human proteins with a K_{D} (K_{D} value) greater than 5×10⁻⁴ mol/litre), preferably it does also not specifically bind to the precursor's target (or binds the precursor target, which may be a non-human protein or a non-protein molecule, with a K_{D} (K_{D} value) greater than 5×10⁻⁴ mol/litre), and preferably it also does not specifically bind to any non-protein molecule, such as nucleic acids (e.g., DNA, RNA), lipids or glycans, preferably it does not specifically bind to any human non-protein molecule (or binds any non-protein molecule with a K_{D} (K_{D} value) greater than 5×10⁻⁴ mol/litre), preferably it also does not specifically bind to any non-protein molecule (such as nucleic acids (e.g., DNA, RNA), glycans, lipids, etc.), to which the building block precursor binds specifically, if any, for instance as determined by cell-binding assay or by surface plasmon resonance (SPR), for instance as described herein and/or in Ober et al. 2001, Intern. Immunology 13:1551-1559, or does not specifically bind to any human cell or binds one or more human cells with a K_{D} (K_{D} value) greater than 5×10⁻⁴ mol/litre, preferably as determined by cell-binding assay or by SPR;
f) optionally, does not specifically bind to any (non-human) molecule which the protein-based carrier building block precursor specifically binds to, such as protein F of RSV, or binds to any (non-human) molecule which the protein-based carrier building block precursor specifically binds to, such as protein F of RSV, with a K_{D} (K_{D} value) greater than 5×10⁻⁴ mol/litre, preferably as determined by cell-binding assay or by SPR;
g) optionally, does not specifically bind to any human cell and/or cell type, or binds to a human cell and/or cell type with a K_{D} (K_{D} value) greater than 5×10−4 mol/litre, preferably as determined by cell-binding assay;
h) optionally, does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein;
i) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein;
j) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein, when it has at least one cargo attached to it (via the at least one conjugation sites or attachment points comprised therein);
k) optionally, does not comprise or consists of an amino acid sequence selected from SEQ ID NO.: 1-34 as depicted on Tables A-1 and A-2 of WO 2016/055656 and/or SEQ ID NO.: 1-12 as depicted on Table A-1 of WO 2010/139808; and
l) optionally, does not comprise or consists of the amino acid sequence as defined in SEQ ID NO.: 214.

In a first aspect, the present technology relates to a molecule comprising at least one protein-based building carrier block, wherein the at least one protein-based carrier building block:
a) comprises at least one conjugation site or attachment point, preferably at least two attachment points or conjugation sites;
b) has a molecular mass of about 2.5 to about 70 kDa, preferably of about 2.5 to about 50 kDa, such as from about 2.5 kDa to less than 50 kDa, more preferably of about 2.5 to about 30 kDa, even more preferably of about 2.5 to about 16 kDa;
c) has a globular 3D structure;
d) has a solubility of 10 mg/ml or more, measured in an aqueous solution at RT, preferably measured in a buffer or water at RT, more preferably in a buffer such as citrate buffer or phosphate-buffered saline (PBS) at pH 7.0 or 7.4, at RT, or histidine buffer at pH 6.5, at RT (comprising histidine (10 mM to 100 mM, such as 10 mM), sucrose (1% to 10%, such as 10%) and, optionally, Tween 80 (0.001% to 1%, such as 0.01%)), or phosphate buffer pH 7.0, at RT (comprising NaH₂PO₄/Na₂HPO₄ (10 and 50 mM, such as 10 mM), sodium chloride (NaCl) (100-150 mM, such as 130 mM NaCl) and, optionally, Tween 80 (0.001% to 1%, such as 0.01%));
e) does not specifically bind to any human protein or binds one or more human proteins with a K_{D} (K_{D} value) greater than 5×10⁻⁴ mol/litre, preferably as determined by cell-binding assay or by surface plasmon resonance (SPR), for instance as described herein and/or in Ober et al. 2001, Intern. Immunology 13:1551-1559, or does not specifically bind to any human cell or binds one or more human cells with a K_{D} (K_{D} value) greater than 5×10⁻⁴ mol/litre, preferably as determined by cell-binding assay or by SPR;
f) optionally, does not specifically bind to any (non-human) molecule which the protein-based carrier building block precursor specifically binds to, such as protein F of RSV, or binds to any (non-human) molecule which the protein-based carrier building block precursor specifically binds to, such as protein F of RSV, with a K_{D} (K_{D} value) greater than 5×10⁻⁴ mol/litre, preferably as determined by cell-binding assay or by SPR;
g) optionally, does not specifically bind to any human cell and/or cell type, or binds to a human cell and/or cell type with a K_{D} (K_{D} value) greater than 5×10⁻⁴ mol/litre, preferably as determined by cell-binding assay;
h) optionally, does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein;
i) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein;
j) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein when it has at least one cargo attached to it (via the at least one conjugation sites or attachment points comprised therein);
k) optionally, does not comprise or consists of an amino acid sequence selected from SEQ ID NO.: 1-34 as depicted on Tables A-1 and A-2 of WO 2016/055656 and/or SEQ ID NO.: 1-12 as depicted on Table A-1 of WO 2010/139808; and
a) optionally, does not comprise or consists of the amino acid sequence as defined in SEQ ID NO.: 214

(EVQLQASGGGLAQPGGSLRLSVTVSGSIDVINNMAWYRQAPGNARELV
ATITSGFSTNYASSVKGRFTISRDNAKKAVYLQMNSLKPEDTADYYSKV
HLIRLGAARAYDYWGQGTQVTVS).

Preferably, in the molecule of the present technology, the at least one protein-based carrier building block does not specifically bind to any non-protein molecule, e.g., to any human non-protein molecule, such as human DNA, human RNA, human lipids or human glycans.

The molecule of the present technology may comprise more than one protein-based carrier building block, such as, e.g., two, three, four, five, six or more protein-based carrier building blocks. These protein-based carrier building blocks may be directly linked to each other, or linked to each other through a linker, as described herein.

Preferably, the at least one protein-based carrier building block comprised in the molecule of the present technology comprises more than one conjugation site or attachment point, preferably at least two conjugation sites or attachment points, such as two conjugation sites or attachment points, or at least three conjugation sites or attachment points, such as three, four, five, six, seven, eight or nine conjugation sites or attachment points, which are preferably reactive groups present in the side chain of a natural or non-natural amino acids comprised in the protein-based carrier building block, or which may be (additionally or alternatively) the N-terminal primary amine and/or the C-terminal carboxylic acid group of the protein-based building block.

For instance, the at least one conjugation site or attachment point comprised in the at least one protein-based carrier building block is a free or capped thiol group, a free or capped hydroxyl group and/or a free or capped primary amine. In a further embodiment, the at least one conjugation site or attachment point comprised in the at least one protein-based carrier building block is a reactive group present in the side chain of a cysteine and/or in the side chain of a tyrosine, and/or in the side chain of a lysine, and/or in the side chain of an ornithine. In another further embodiment, the at least one protein-based building block comprises a N- and/or a C-terminal Cys and/or a N- and/or a C-terminal Tyr, preceded or followed by a (GG) or (G₄S₁)₁₋₃GG sequence, such as CGG-, -GGC, YGG-, -GGY, -(G₄S₁)₁₋₃GGY, Y(G₄S₁)₁₋₃GG-, YGG(S₁G₄)₁₋₃-, or YGG(G₄S₁)₁₋₃-.

Preferably, the at least one protein-based carrier building block present in the molecule of the present technology is (i) a building block based on small globular non-human proteins, such as an ISVD-based building block, a DARPin-based building block, an affibody-based building block or an affitin-based building block or (ii) a building block based on small globular human proteins, such as cyclin-dependent kinase subunit 1 (CDK-1).

In one embodiment, the least one protein-based building block is derived from a heavy chain ISVD, preferably from a V_{H}, V_{HH}, including a camelized V_{H} or humanized V_{HH}. In another embodiment, the at least one protein-based building block is derived from an ISVD belonging to the “V_{H}3 class”, preferably wherein the resulting building block comprises at least one (preferably engineered) cysteine, at least one (preferably engineered) lysine, at least one non-natural amino acid and/or at least one (preferably engineered) tyrosine at one or more solvent-accessible positions of the protein-based building block.

In another embodiment, the least one protein-based building block is derived from RSV001A04, SEQ ID NO.: 179.

In one embodiment, the at least one protein-based building block is an ISVD-based building block which comprises a Leu or a Gln, preferably a Leu at position 108, according to Kabat numbering, preferably wherein the ISVD-based building block comprises a Val or a Leu, preferably a Val at position 11 and/or a Val, a Thr or a Leu, preferably a Leu at position 89, according to Kabat numbering.

In another embodiment, the at least one protein-based building block comprises or, alternatively, consists of SEQ ID NO.: 186:

X₁VX₂LX₃EX₄X₅GX₆X₇X₈X₉X₁₀X₁₁GX₁₂X₁₃X₁₄IX₁₅CX₁₆AX₁₇X₁₈X₁₉X₂₀L
X₂₁X₂₂X₂₃VLGWFRX₂₄AX₂₅X₂₆X₂₇X₂₈X₂₉X₃₀FVAAINX₃₁X₃₂X₃₃X₃₄X₃₅X₃₆
X₃₇X₃₈PX₃₉X₄₀VX₄₁X₄₂X₄₃FX₄₄IX₄₅X₄₆X₄₇X₄₈X₄₉X₅₀X₅₁TGX₅₂LX₅₃MX₅₄
X₅₅LX₅₆X₅₇X₅₈DX₅₉AX₆₀YX₆₁CGAGX₆₂PX₆₃X₆₄X₆₅X₆₆AYX₆₇X₆₈X₆₉X₇₀S
YX₇₁X₇₂X₇₃GX₇₄X₇₅TX₇₆VX₇₇VX₇₈X₇₉X₈₀X₈₁X₈₂,

X₁ (position 1 according to Kabat numbering) can be Glu or any amino acid with a reactive group in its side chain, such as cysteine;
X₂ (position 3 according to Kabat numbering) can be Gln or any amino acid with a reactive group in its side chain, such as cysteine;
X₃ (position 5 according to Kabat numbering) can be Val or any amino acid with a reactive group in its side chain, such as cysteine;
X₄ (position 7 according to Kabat numbering) can be Ser or any amino acid with a reactive group in its side chain, such as cysteine;
X₅ (position 8 according to Kabat numbering) can be Gly or any amino acid with a reactive group in its side chain, such as cysteine;
X₆ (position 10 according to Kabat numbering) can be Gly or any amino acid with a reactive group in its side chain, such as cysteine;
X₇ (position 11 according to Kabat numbering) can be Leu, Val Ser, Met, Trp, Phe, Thr, Gln, Glu, Ala, Arg, Gly, Lys, Tyr, Asn, Pro or Ile, preferably Leu or Val, or any other amino acid with a reactive group in its side chain, such as cysteine;
X₈ (position 12 according to Kabat numbering) can be Val or any amino acid with a reactive group in its side chain, such as cysteine;
X₉ (position 13 according to Kabat numbering) can be Gln or any amino acid with a reactive group in its side chain, such as cysteine;
X₁₀ (position 14 according to Kabat numbering) can be Ala or any amino acid with a reactive group in its side chain, such as cysteine;
X₁₁ (position 15 according to Kabat numbering) can be Gly or any amino acid with a reactive group in its side chain, such as cysteine;
X₁₂ (position 17 according to Kabat numbering) can be Ser or any amino acid with a reactive group in its side chain, such as cysteine;
X₁₃ (position 18 according to Kabat numbering) can be Leu or any amino acid with a reactive group in its side chain, such as cysteine;
X₁₄ (position 19 according to Kabat numbering) can be Ser or any amino acid with a reactive group in its side chain, such as cysteine;
X₁₅: (position 21 according to Kabat numbering) can be Ser or any amino acid with a reactive group in its side chain, such as cysteine;
X₁₆: (position 23 according to Kabat numbering) can be Ala or any amino acid with a reactive group in its side chain, such as cysteine;
X₁₇: (position 25 according to Kabat numbering) can be Ser or any amino acid with a reactive group in its side chain, such as cysteine;
X₁₈: (position 26 according to Kabat numbering) can be Gly or any amino acid with a reactive group in its side chain, such as cysteine;
X₁₉: (position 27 according to Kabat numbering) can be Gly or any amino acid with a reactive group in its side chain, such as cysteine;
X₂₀: (position 28 according to Kabat numbering) can be Ser or any amino acid with a reactive group in its side chain, such as cysteine;
X₂₁: (position 30 according to Kabat numbering) can be Ser or any amino acid with a reactive group in its side chain, such as cysteine;
X₂₂: (position 31 according to Kabat numbering) can be Asn or any amino acid with a reactive group in its side chain, such as cysteine;
X₂₃: (position 32 according to Kabat numbering) can be Tyr or any amino acid with a reactive group in its side chain, such as cysteine;
X₂₄: (position 39 according to Kabat numbering) can be Gln or any amino acid with a reactive group in its side chain, such as cysteine;
X₂₅: (position 41 according to Kabat numbering) can be Pro or any amino acid with a reactive group in its side chain, such as cysteine;
X₂₆: (position 42 according to Kabat numbering) can be Gly or any amino acid with a reactive group in its side chain, such as cysteine;
X₂₇: (position 43 according to Kabat numbering) can be Lys or any amino acid with a reactive group in its side chain, such as cysteine;
X₂₈: (position 44 according to Kabat numbering) can be Glu or any amino acid with a reactive group in its side chain, such as cysteine;
X₂₉: (position 45 according to Kabat numbering) can be Arg or any amino acid with a reactive group in its side chain, such as cysteine;
X₃₀: (position 46 according to Kabat numbering) can be Glu or any amino acid with a reactive group in its side chain, such as cysteine;
X₃₁: (position 52a according to Kabat numbering) can be Trp or any amino acid with a reactive group in its side chain, such as cysteine;
X₃₂: (position 53 according to Kabat numbering) can be Arg or any amino acid with a reactive group in its side chain, such as cysteine;
X₃₃: (position 54 according to Kabat numbering) can be Gly or any amino acid with a reactive group in its side chain, such as cysteine;
X₃₄: (position 55 according to Kabat numbering) can be Asp or any amino acid with a reactive group in its side chain, such as cysteine;
X₃₅: (position 56 according to Kabat numbering) can be Ile or any amino acid with a reactive group in its side chain, such as cysteine;
X₃₆: (position 57 according to Kabat numbering) can be Thr or any amino acid with a reactive group in its side chain, such as cysteine;
X₃₇: (position 58 according to Kabat numbering) can be Ile or any amino acid with a reactive group in its side chain, such as cysteine;
X₃₈: (position 59 according to Kabat numbering) can be Gly or any amino acid with a reactive group in its side chain, such as cysteine;
X₃₉: (position 61 according to Kabat numbering) can be Pro or any amino acid with a reactive group in its side chain, such as cysteine;
X₄₀: (position 62 according to Kabat numbering) can be Asn or any amino acid with a reactive group in its side chain, such as cysteine;
X₄₁: (position 64 according to Kabat numbering) can be Glu or any amino acid with a reactive group in its side chain, such as cysteine;
X₄₂: (position 65 according to Kabat numbering) can be Gly or any amino acid with a reactive group in its side chain, such as cysteine;
X₄₃: (position 66 according to Kabat numbering) can be Arg or any amino acid with a reactive group in its side chain, such as cysteine;
X₄₄: (position 68 according to Kabat numbering) can be Thr or any amino acid with a reactive group in its side chain, such as cysteine;
X₄₅: (position 70 according to Kabat numbering) can be Ser or any amino acid with a reactive group in its side chain, such as cysteine;
X₄₆: (position 71 according to Kabat numbering) can be Arg or any amino acid with a reactive group in its side chain, such as cysteine;
X₄₇: (position 72 according to Kabat numbering) can be Asp or any amino acid with a reactive group in its side chain, such as cysteine;
X₄₈: (position 73 according to Kabat numbering) can be Asn or any amino acid with a reactive group in its side chain, such as cysteine;
X₄₉: (position 74 according to Kabat numbering) can be Ala or any amino acid with a reactive group in its side chain, such as cysteine;
X₅₀: (position 75 according to Kabat numbering) can be Lys or any amino acid with a reactive group in its side chain, such as cysteine;
X₅₁: (position 76 according to Kabat numbering) can be Asn or any amino acid with a reactive group in its side chain, such as cysteine;
X₅₂: (position 79 according to Kabat numbering) can be Tyr or any amino acid with a reactive group in its side chain, such as cysteine;
X₅₃: (position 81 according to Kabat numbering) can be Gln or any amino acid with a reactive group in its side chain, such as cysteine;
X₅₄: (position 82a according to Kabat numbering) can be Asn or any amino acid with a reactive group in its side chain, such as cysteine;
X₅₅: (position 82b according to Kabat numbering) can be Ser or any amino acid with a reactive group in its side chain, such as cysteine;
X₅₆: (position 83 according to Kabat numbering) can be Ala or any amino acid with a reactive group in its side chain, such as cysteine;
X₅₇: (position 84 according to Kabat numbering) can be Pro or any amino acid with a reactive group in its side chain, such as cysteine;
X₅₈: (position 85 according to Kabat numbering) can be Asp or any amino acid with a reactive group in its side chain, such as cysteine;
X₅₉: (position 87 according to Kabat numbering) can be Thr or any amino acid with a reactive group in its side chain, such as cysteine;
X₆₀: (position 89 according to Kabat numbering) can be Leu, Val Ser, Met, Trp, Phe, Thr, Gln, Glu, Ala, Arg, Gly, Lys, Tyr, Asn, Pro or Ile, preferably Leu or Val, or any other amino acid with a reactive group in its side chain, such as cysteine;
X₆₁: (position 91 according to Kabat numbering) can be Tyr or any amino acid with a reactive group in its side chain, such as cysteine;
X₆₂: (position 96 according to Kabat numbering) can be Thr or any amino acid with a reactive group in its side chain, such as cysteine;
X₆₃: (position 98 according to Kabat numbering) can be Leu or any amino acid with a reactive group in its side chain, such as cysteine;
X₆₄: (position 99 according to Kabat numbering) can be Asn or any amino acid with a reactive group in its side chain, such as cysteine;
X₆₅: (position 100 according to Kabat numbering) can be Pro or any amino acid with a reactive group in its side chain, such as cysteine;
X₆₆: (position 100a according to Kabat numbering) can be Gly or any amino acid with a reactive group in its side chain, such as cysteine;
X₆₇: (position 100d according to Kabat numbering) can be Ile or any amino acid with a reactive group in its side chain, such as cysteine;
X₆₈: (position 100e according to Kabat numbering) can be Tyr or any amino acid with a reactive group in its side chain, such as cysteine;
X₆₉: (position 100f according to Kabat numbering) can be Asp or any amino acid with a reactive group in its side chain, such as cysteine;
X₇₀: (position 100g according to Kabat numbering) can be Trp or any amino acid with a reactive group in its side chain, such as cysteine;
X₇₁: (position 101 according to Kabat numbering) can be Asp or any amino acid with a reactive group in its side chain, such as cysteine;
X₇₂: (position 102 according to Kabat numbering) can be Tyr or any amino acid with a reactive group in its side chain, such as cysteine;
X₇₃: (position 103 according to Kabat numbering) can be Trp or any amino acid with a reactive group in its side chain, such as cysteine;
X₇₄: (position 105 according to Kabat numbering) can be Arg or any amino acid with a reactive group in its side chain, such as cysteine;
X₇₅: (position 106 according to Kabat numbering) can be Gly or any amino acid with a reactive group in its side chain, such as cysteine;
X₇₆: (position 108 according to Kabat numbering) can be Gln, Leu, Arg, Pro, Glu, Lys, Ser, Thr, Met, Ala or His; preferably Gln or Leu or any other amino acid with a reactive group in its side chain, such as cysteine;
X₇₇: (position 110 according to Kabat numbering) can be Thr or any amino acid with a reactive group in its side chain, such as cysteine;
X₇₈: (position 112 according to Kabat numbering) can be Ser or any amino acid with a reactive group in its side chain, such as cysteine.
X₇₉: (position 113 according to Kabat numbering) can be Ser or any amino acid with a reactive group in its side chain, such as cysteine;
X₈₀: is absent or Gly;
X₈₁: is absent or Gly;
X₈₂: is absent or Cys,
or a sequence which has 80% or more identity with SEQ ID NO.: 186, preferably a sequence which has 85% or more, 90% or more, 95% or more, 97% or more or 99% or more sequence identity with SEQ ID NO.: 186, provided that the building block has a globular 3D structure, is soluble, has a size (molecular mass) of about 2.5 to about 70 kDa, such as about 2.5 to about 50 kDa, such as about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described herein.

In another embodiment, the at least one protein-based building block is a DARPin-based building block, preferably derived from the DARPin K27 as defined in SEQ ID NO.: 187.

SEQ ID NO.: 187:
DLGKKLLEAARAGQDDEVRILMANGADVNAHDTFGFTPLHLAALYGHLE
IVEVLLKNGADVNADDSYGRTPLHLAAMRGHLEIVEVLLKYGADVNAAD
EEGRTPLHLAAKRGHLEIVEVLLKNGADVNAQDKFGKTAFDISIDNGNE
DLAEILQKL

In one embodiment, the protein-based building block is a DARPin-based building block which comprises, or alternatively, consists of, SEQ ID NO.: 188:

X₁X₂GX₃X₄LLX₅AAX₆X₇X₈X₉X₁₀X₁₁X₁₂VX₁₃X₁₄LMX₁₅X₁₆X₁₇AX₁₈VX₁₉A
X₂₀X₂₁X₂₂X₂₃GX₂₄TPLHLAAX₂₅X₂₆X₂₇X₂₈X₂₉X₃₀IVX₃₁VLLX₃₂X₃₃X₃₄A
X₃₅VX₃₆AX₃₇DX₃₈X₃₉GATPLHLAAX₄₀X₄₁X₄₂X₄₃X₄₄X₄₅IVX₄₆VLLX₄₇X₄₈
X₄₉AX₅₀VX₅₁AX₅₂DX₅₃X₅₄GATPLHX₅₅AAX₅₆X₅₇X₅₈X₅₉X₆₀X₆₁IVX₆₂X₆₃L
X₆₄X₆₅X₆₆X₆₇AX₆₈X₆₉X₇₀AX₇₁DX₇₂X₇₃X₇₄X₇₅TAX₇₆X₇₇ISX₇₈X₇₉X₈₀X₈₁
X₈₂X₈₃X₈₄LAX₈₅X₈₆LX₈₇X₈₈X₈₉X₉₀,

wherein:
X₁ can be Asp or any amino acid with a reactive group in its side chain, such as cysteine;
X₂ can be Leu or any amino acid with a reactive group in its side chain, such as cysteine;
X₃ can be Lys or any amino acid with a reactive group in its side chain, such as cysteine;
X₄ can be Lys or any amino acid with a reactive group in its side chain, such as cysteine;
X₅ can be Glu or any amino acid with a reactive group in its side chain, such as cysteine;
X₆ can be Arg or any amino acid with a reactive group in its side chain, such as cysteine;
X₇ can be Ala or any amino acid with a reactive group in its side chain, such as cysteine;
X₈ can be Gly or any amino acid with a reactive group in its side chain, such as cysteine;
X₉ can be Gln or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Arg or any amino acid with a reactive group in its side chain, such as cysteine;
X₁₄ can be Ile or any amino acid with a reactive group in its side chain, such as cysteine;
X₁₅ can be Ala or any amino acid with a reactive group in its side chain, such as cysteine;
X₁₆ can be Asn or any amino acid with a reactive group in its side chain, such as cysteine;
X₁₇ can be Gly or any amino acid with a reactive group in its side chain, such as cysteine;
X₁₈ can be Asp or any amino acid with a reactive group in its side chain, such as cysteine;
X₁₉ can be Asn or any amino acid with a reactive group in its side chain, such as cysteine;
X₂₀ can be His or any amino acid with a reactive group in its side chain, such as cysteine;
X₂₁ can be Asp or any amino acid with a reactive group in its side chain, such as cysteine;
X₂₂ can be Thr or any amino acid with a reactive group in its side chain, such as cysteine;
X₂₃ can be Phe or any amino acid with a reactive group in its side chain, such as cysteine;
X₂₄ can be Phe or any amino acid with a reactive group in its side chain, such as cysteine;
X₂₅ can be Leu or any amino acid with a reactive group in its side chain, such as cysteine;
X₂₆ can be Tyr or any amino acid with a reactive group in its side chain, such as cysteine;
X₂₇ can be Gly or any amino acid with a reactive group in its side chain, such as cysteine;
X₂₈ can be His or any amino acid with a reactive group in its side chain, such as cysteine
X₂₉ can be Leu or any amino acid with a reactive group in its side chain, such as cysteine
X₃₀ can be Glu or any amino acid with a reactive group in its side chain, such as cysteine
X₃₁ can be Glu or any amino acid with a reactive group in its side chain, such as cysteine
X₃₂ can be Lys or any amino acid with a reactive group in its side chain, such as cysteine
X₃₃ can be Asn or any amino acid with a reactive group in its side chain, such as cysteine
X₃₄ can be Gly or any amino acid with a reactive group in its side chain, such as cysteine
X₃₅ can be Asp or any amino acid with a reactive group in its side chain, such as cysteine
X₃₆ can be Asn or any amino acid with a reactive group in its side chain, such as cysteine
X₃₇ can be Asp or any amino acid with a reactive group in its side chain, such as cysteine
X₃₈ can be Ser or any amino acid with a reactive group in its side chain, such as cysteine
X₃₉ can be Tyr or any amino acid with a reactive group in its side chain, such as cysteine;
X₄₀ can be Met or any amino acid with a reactive group in its side chain, such as cysteine;
X₄₁ can be Arg or any amino acid with a reactive group in its side chain, such as cysteine;
X₄₂ can be Gly or any amino acid with a reactive group in its side chain, such as cysteine;
X₄₃ can be His or any amino acid with a reactive group in its side chain, such as cysteine;
X₄₄ can be Leu or any amino acid with a reactive group in its side chain, such as cysteine;
X₄₅ can be Glu or any amino acid with a reactive group in its side chain, such as cysteine;
X₄₆ can be Glu or any amino acid with a reactive group in its side chain, such as cysteine;
X₄₇ can be Lys or any amino acid with a reactive group in its side chain, such as cysteine;
X₄₈ can be Tyr or any amino acid with a reactive group in its side chain, such as cysteine;
X₄₉ can be Gly or any amino acid with a reactive group in its side chain, such as cysteine;
X₅₀ can be Asp or any amino acid with a reactive group in its side chain, such as cysteine;
X₅₁ can be Asn or any amino acid with a reactive group in its side chain, such as cysteine;
X₅₂ can be Ala or any amino acid with a reactive group in its side chain, such as cysteine;
X₅₃ can be Glu or any amino acid with a reactive group in its side chain, such as cysteine;
X₅₄ can be Glu or any amino acid with a reactive group in its side chain, such as cysteine;
X₅₅ can be Leu or any amino acid with a reactive group in its side chain, such as cysteine;
X₅₆ can be Lys or any amino acid with a reactive group in its side chain, such as cysteine;
X₅₇ can be Ala or any amino acid with a reactive group in its side chain, such as cysteine;
X₅₈ can be Gly or any amino acid with a reactive group in its side chain, such as cysteine;
X₅₉ can be His or any amino acid with a reactive group in its side chain, such as cysteine;
X₆₀ can be Leu or any amino acid with a reactive group in its side chain, such as cysteine;
X₆₁ can be Glu or any amino acid with a reactive group in its side chain, such as cysteine;
X₆₂ can be Glu or any amino acid with a reactive group in its side chain, such as cysteine;
X₆₃ can be Val or any amino acid with a reactive group in its side chain, such as cysteine;
X₆₄ can be Leu or any amino acid with a reactive group in its side chain, such as cysteine;
X₆₅ can be Lys or any amino acid with a reactive group in its side chain, such as cysteine;
X₆₆ can be Asn or any amino acid with a reactive group in its side chain, such as cysteine;
X₆₇ can be Gly or any amino acid with a reactive group in its side chain, such as cysteine;
X₆₈ can be Asp or any amino acid with a reactive group in its side chain, such as cysteine;
X₆₉ can be Val or any amino acid with a reactive group in its side chain, such as cysteine;
X₇₀ can be Asn or any amino acid with a reactive group in its side chain, such as cysteine;
X₇₁ can be Gln or any amino acid with a reactive group in its side chain, such as cysteine;
X₇₂ can be Lys or any amino acid with a reactive group in its side chain, such as cysteine;
X₇₃ can be Phe or any amino acid with a reactive group in its side chain, such as cysteine;
X₇₄ can be Gly or any amino acid with a reactive group in its side chain, such as cysteine;
X₇₅ can be Lys or any amino acid with a reactive group in its side chain, such as cysteine;
X₇₆ can be Phe or any amino acid with a reactive group in its side chain, such as cysteine;
X₇₇ can be Asp or any amino acid with a reactive group in its side chain, such as cysteine;
X₇₈ can be Ile or any amino acid with a reactive group in its side chain, such as cysteine;
X₇₉ can be Asp or any amino acid with a reactive group in its side chain, such as cysteine;
X₈₀ can be Asn or any amino acid with a reactive group in its side chain, such as cysteine;
X₈₁ can be Gly or any amino acid with a reactive group in its side chain, such as cysteine;
X₈₂ can be Asn or any amino acid with a reactive group in its side chain, such as cysteine;
X₈₃ can be Glu or any amino acid with a reactive group in its side chain, such as cysteine;
X₈₄ can be Asp or any amino acid with a reactive group in its side chain, such as cysteine;
X₈₅ can be Glu or any amino acid with a reactive group in its side chain, such as cysteine;
X₈₆ can be Ile or any amino acid with a reactive group in its side chain, such as cysteine;
X₈₇ can be Gln or any amino acid with a reactive group in its side chain, such as cysteine;
X₈₈ can be Lys or any amino acid with a reactive group in its side chain, such as cysteine
X₈₉ is absent or Leu;
X₉₀ is absent or Cys,
or a sequence which has 80% or more identity with SEQ ID NO.: 188, preferably a sequence which has 85% or more, 90% or more, 95% or more, 97% or more or 99% or more sequence identity with SEQ ID NO.: 188, provided that the building block has a globular 3D structure, is soluble, has a size (molecular mass) of about 2.5 to about 70 kDa, such as about 2.5 to about 50 kDa, such as about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described herein, in particular does not specifically bind to human KRAS protein (GTPase KRas, EC: 3.6.5.2, primary accession number P01116, see also Lim S., et al., “Exquisitely specific anti-KRAS biodegraders inform on the cellular prevalence of nucleotide-loaded states”, ACS Cent. Sci. 2021, 7, 2, 274-291).

In another embodiment, the at least one protein-based building block is a small globular human protein-based building bock, preferably derived from the polypeptide as defined in SEQ ID NO.: 190.

SEQ ID NO.: 190
SHKQIYYSDKYDDEEFEYRHVMLPKDIAKLVPKTHLMSESEWRNLGVQQ
SQGWVHYMIHEPEPHILLFRRPLPKKPKK

In one embodiment, the protein-based building block is a small globular human protein-based building bock which comprises, or alternatively, consists of, SEQ ID NO.: 191:

X₁X₂X₃X₄₁X₅X₆SX₇X₈X₉X₁₀X₁₁X₁₂X₁₃X₁₄X₁₅X₁₆X₁₇X₁₈VX₁₉LPX₂₀X₂₁X₂₂
AX₂₃X₂₄VX₂₅X₂₃bX₂₄bX₂₅bX₂₆MX₂₇X₂₈X₂₉X₃₀WX₃₁X₃₂LX₃₃VX₃₄QX₃₅X₃₆
X₃₇WX₃₈HX₃₉X₄₀X₄₁X₄₂X₄₃X₄₄X₄₅X₄₆X₄₇ILLFX₄₈X₄₉X₅₀X₅₁X₅₂X₅₃X₅₄
X₅₅X₅₆X₅₇,

wherein
X₁ can be Ser or any amino acid with a reactive group in its side chain, such as cysteine;
X₂ can be His or any amino acid with a reactive group in its side chain, such as cysteine;
X₃ can be Lys or any amino acid with a reactive group in its side chain, such as cysteine;
X₄ can be Gln or any amino acid with a reactive group in its side chain, such as cysteine;
X₅ can be Tyr or any amino acid with a reactive group in its side chain, such as cysteine;
X₆ can be Tyr or any amino acid with a reactive group in its side chain, such as cysteine;
X₇ can be Asp or any amino acid with a reactive group in its side chain, such as cysteine;
X₈ can be Lys or any amino acid with a reactive group in its side chain, such as cysteine;
X₉ can be Tyr or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Glu or any amino acid with a reactive group in its side chain, such as cysteine;
X₁₄ can be Phe or any amino acid with a reactive group in its side chain, such as cysteine;
X₁₅ can be Glu or any amino acid with a reactive group in its side chain, such as cysteine;
X₁₆ can be Tyr or any amino acid with a reactive group in its side chain, such as cysteine;
X₁₇ can be Arg or any amino acid with a reactive group in its side chain, such as cysteine;
X₁₈ can be His or any amino acid with a reactive group in its side chain, such as cysteine;
X₁₉ can be Met or any amino acid with a reactive group in its side chain, such as cysteine;
X₂₀ can be Lys or any amino acid with a reactive group in its side chain, such as cysteine;
X₂₁ can be Asp or any amino acid with a reactive group in its side chain, such as cysteine;
X₂₂ can be Ile or any amino acid with a reactive group in its side chain, such as cysteine;
X₂₃ can be Lys or any amino acid with a reactive group in its side chain, such as cysteine;
X₂₄ can be Leu or any amino acid with a reactive group in its side chain, such as cysteine;
X₂₅ can be Pro or any amino acid with a reactive group in its side chain, such as cysteine;
X₂₃b can be Lys or any amino acid with a reactive group in its side chain, such as cysteine;
X₂₄b can be Thr or any amino acid with a reactive group in its side chain, such as cysteine;
X₂₅b can be His or any amino acid with a reactive group in its side chain, such as cysteine;
X₂₆ can be Leu or any amino acid with a reactive group in its side chain, such as cysteine;
X₂₇ can be Ser or any amino acid with a reactive group in its side chain, such as cysteine;
X₂₈ can be Glu or any amino acid with a reactive group in its side chain, such as cysteine;
X₂₉ can be Ser or any amino acid with a reactive group in its side chain, such as cysteine;
X₃₀ can be Glu or any amino acid with a reactive group in its side chain, such as cysteine;
X₃₁ can be Arg or any amino acid with a reactive group in its side chain, such as cysteine;
X₃₂ can be Asn or any amino acid with a reactive group in its side chain, such as cysteine;
X₃₃ can be Gly or any amino acid with a reactive group in its side chain, such as cysteine;
X₃₄ can be Gln or any amino acid with a reactive group in its side chain, such as cysteine;
X₃₅ can be Ser or any amino acid with a reactive group in its side chain, such as cysteine;
X₃₆ can be Gln or any amino acid with a reactive group in its side chain, such as cysteine;
X₃₇ can be Gly or any amino acid with a reactive group in its side chain, such as cysteine;
X₃₈ can be Val or any amino acid with a reactive group in its side chain, such as cysteine;
X₃₉ can be Tyr or any amino acid with a reactive group in its side chain, such as cysteine;
X₄₀ can be Met or any amino acid with a reactive group in its side chain, such as cysteine;
X₄₁ can be Ile or any amino acid with a reactive group in its side chain, such as cysteine;
X₄₂ can be His or any amino acid with a reactive group in its side chain, such as cysteine;
X₄₃ can be Glu or any amino acid with a reactive group in its side chain, such as cysteine;
X₄₄ can be Pro or any amino acid with a reactive group in its side chain, such as cysteine;
X₄₅ can be Glu or any amino acid with a reactive group in its side chain, such as cysteine;
X₄₆ can be Pro or any amino acid with a reactive group in its side chain, such as cysteine;
X₄₇ can be His or any amino acid with a reactive group in its side chain, such as cysteine;
X₄₈ can be Arg or any amino acid with a reactive group in its side chain, such as cysteine;
X₄₉ can be Arg or any amino acid with a reactive group in its side chain, such as cysteine;
X₅₀ can be Pro or any amino acid with a reactive group in its side chain, such as cysteine;
X₅₁ can be Leu or any amino acid with a reactive group in its side chain, such as cysteine;
X₅₂ can be Pro or any amino acid with a reactive group in its side chain, such as cysteine;
X₅₃ can be Lys or any amino acid with a reactive group in its side chain, such as cysteine;
X₅₄ can be Lys or any amino acid with a reactive group in its side chain, such as cysteine;
X₅₅ can be Pro or any amino acid with a reactive group in its side chain, such as cysteine;
X₅₆ can be Lys or any amino acid with a reactive group in its side chain, such as cysteine;
X₅₇ can be Lys or any amino acid with a reactive group in its side chain, such as cysteine,
or a sequence which has 80% or more identity with SEQ ID NO.: 191, preferably a sequence which has 85% or more, 90% or more, 95% or more, 97% or more or 99% or more sequence identity with SEQ ID NO.: 191, provided that the building block has a globular 3D structure, is soluble, has a size (molecular mass) of about 2.5 to about 70 kDa, such as from about 2.5 to about 50 kDa, such as from about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described herein.

For instance, the at least one protein-based building block may be selected from SEQ ID NO.: 80-105, 175, 199, 208, 222-224.

In one embodiment, the molecule of the present technology comprises at least one protein-based carrier building block as defined herein and at least one further moiety or cargo attached to the attachment point or conjugation site, wherein the at least one further moiety or cargo is selected from:
a) a half-life extending (HLE) moiety, such as PEG, and/or
b) a targeting moiety, such as an EGFR-targeting moiety, e.g., GE11 peptide or an anti-EGFR VHH; and/or
c) a therapeutic moiety or precursor therefrom, e.g., such as an DR5-binding molecule;
d) an imaging moiety, such as deferoxamine (DFO);
e) a toxic moiety, such as DM4 or cryptophycin;
f) nucleic acids such as siRNA;
g) vitamins, such as folate;
h) Toll-like receptor agonists, such as resiquimod;
i) glycans, such as bis mannose 6 phosphate (bisM6P) or mannose 6 phosphate (M6P); and/or
j) lipids, such as short-chain fatty acids.

In one embodiment, the at least one half-life extending moiety is an albumin-binding ISVD, wherein the albumin-binding ISVD is preferably selected from SEQ ID NOs: 50-64 and 106, more preferably SEQ ID NO.: 63 or SEQ ID NO.: 106, or a sequence with at least 70%, preferably at least 80%, more preferably at least 90% and even more preferably at least 95% identity with SEQ ID NOs: 50-64 and/or 106. In a further embodiment, the at least one half-life extending moiety is a linear or branched polyethylene glycol moiety with a molecular weight of about 1-60 kDa, preferably with a weight of about 1-15 kDa, such as about 14 or 15 kDa, or of about 1-10 kDa, such as 5 or 10 kDa.

In one embodiment, the at least one protein-based carrier building block and the at least one further moiety or cargo are directly linked to each other. In a further embodiment, they are linked to each other through a peptide linker, preferably wherein the peptide linker is selected from SEQ ID NO.: 158-169 or 193-196, more preferably SEQ ID NO.: 163. Other linkers may be used, such as APN-maleimide linkers, as defined below and exemplified in the examples.

For instance, the molecule of the present technology may comprise or, alternatively consist of, any one of SEQ ID NOs.: 107-127, SEQ ID NOs.: 170-174, 176 or 200.

The present technology also provides a nucleic acid encoding the molecule of the present technology (or part of the molecule of the present technology). In addition, the present technology provides a vector comprising the nucleic acid of the present technology, and a composition comprising the molecule of the present technology, such as a pharmaceutical composition.

Furthermore, the present technology relates to the molecule or composition of the present technology for use in medicine, in particular for use in the (prophylactic or therapeutic) treatment of diseases and or disorders, such as autoimmune/inflammatory diseases, cancer and/or infectious diseases.

### BRIEF DESCRIPTION OF THE FIGURES

FIG. 1. Amino acid sequence of ISVD RSV001A04 (SEQ ID NO.: 179).

FIG. 2. Amino acid sequence of K27m (without the C-terminal L), SEQ ID NO.: 68.

FIG. 3. Amino acid sequence of the CKS1-building block precursor (SEQ ID NO.: 190).

FIG. 4. Conjugation using an APN-maleimide ‘bifunctional’ linker. The carrier (protein-based building block comprised in the molecule) comprises at least one attachment point or conjugation site (represented as “—SH” in FIG. 4). The APN-maleimide ‘bifunctional’ linker can be first attached to the conjugation site present in the carrier. Then, the cargo (represented as “DR5-SH” in FIG. 4) can be attached to the other side of the APN-maleimide ‘bifunctional’ linker. Hence, the cargo has been attached or conjugated to the carrier through an APN-maleimide ‘bifunctional’ linker.

FIGS. 5A and 5B. Conjugability check of cysteine-engineered ISVDs-based carrier building blocks using Mass Spectrometry Deconvoluted Mass Spectrum of Mal-APN conjugation onto the molecule T028100075, comprising an ISVD-based carrier building block with one attachment point or conjugation site (“ISVD179-APN”, SEQ ID NO.: 176, FIG. 5A) and onto the molecule T028100069, comprising an ISVD-based carrier building block with three attachment points or conjugation sites (“ISVD107-APN”, SEQ ID NO.: 107, FIG. 5B). Mass Spec analysis was carried out using electrospray ionization (ESI) with online reverse phase column (RPC) for clean-up of the sample.

FIGS. 6A and 6B. Schematic representation of the molecules of Example 5. FIGS. 6A-6B show the multivalent molecules comprising an HLE moiety (“ALB23”, which represents SEQ ID NO.: 106), a protein-based carrier building block (“CARRIER”, which represents SEQ ID NO.: 80) and three cargos (“DR5”) attached to three conjugation sites, which are three —SH groups of cysteines present at solvent-accessible positions. In FIG. 6A, each cargo (“DR5”) is a DR5-GGC ISVD (“monovalent”, DR5-GGC, SEQ ID NO.: 177). In FIG. 6B, each cargo (“DR5-DR5-DR5”) comprises three DR5 ISVDs (“trivalent”, DR5-DR5-DR5-GGC, SEQ ID NO.: 178).

FIG. 7. SDS-PAGE gel analysis of the conjugations performed in Example 5. Lane 3 shows the 3× conjugation status of the “trivalent” (DR5-DR5-DR5-GGC) cargo attached to the three conjugation sites on the building block as defined by SEQ ID NO.: 176. Lane 5 shown the 3× conjugation status of the “monovalent” (DR5-GGC) cargo attached to the three conjugation sites on the building block as defined by SEQ ID NO.: 176. The results of the control are not shown in FIG. 7. Lane 4 is a protein ladder.

FIG. 8. Caspase 3/7 assay. Medium containing the indicated concentrations of DR5 agonists, as described below, was added, followed by 5 μM CellEvent™ Caspase-3/7 Green Detection Reagent. Real-time data acquisition and analysis were performed, and the caspase 3/7 signal was normalized for cell confluency at 24 h timepoint. “DR5 Trivalent format” refers to the trivalent DR5-GGC molecule as defined in SEQ ID NO.: 178; “DR5 Bivalent format” refers to the molecule as described in SEQ ID NO.: 207; “CARGO Nonavalent DR5” refers to a molecule comprising a HLE moiety (“ALB”, SEQ ID NO.: 106), an ISVD-based carrier building block (“CARRIER”, SEQ ID NO.: 107) with three conjugation sites (at positions 43, 100f and 105, according to Kabat) and three cargos attached via an APN-maleimide linker to the three conjugation sites, wherein each cargo comprises three DR5 ISVDs (SEQ ID NO.: 178); “CARGO Trivalent DR5” refers to a molecule comprising a HLE moiety (“ALB”, SEQ ID NO.: 106), an ISVD-based carrier building block (“CARRIER”, SEQ ID NO.: 107) with three conjugation sites (at positions 43, 100f and 105, according to Kabat) and three cargos attached via an APN-maleimide linker to the three conjugation sites, wherein each cargo comprises one DR5 ISVD (SEQ ID NO.: 177); “CARGO control” refers to the control molecule, which comprises a HLE moiety (“ALB”, SEQ ID NO.: 106), a control carrier building block (“RSV”, SEQ ID NO.: 176) with a C-terminal Cys to which a molecule comprising three DR5 ISVDs is attached via an APN-maleimide linker.

FIG. 9. siRNA conjugation using the molecule described in SEQ ID NO.: 108. “DOL3” refers to the molecule described in SEQ ID NO.: 108 with three siRNA molecules attached to the three conjugation sites comprised in the protein-based building block of this molecule. “DOL2” refers to the molecule described in SEQ ID NO.: 108 with two siRNA molecules attached to two conjugation sites, and “DOL1” refers to the molecule described in SEQ ID NO.: 108 with one siRNA molecule attached to one conjugation site. “T028100070” refers to the molecule described in SEQ ID NO.: 108 on its own, and “T028100070+TCEP” refers to the molecule described in SEQ ID NO.: 108 in its reduced form with TCEP.

FIG. 10. Combination of PEGylation (5, 10 or 20 kDa PEG) and radiolabeling (⁸⁹Zn) of molecules as described in SEQ ID NO.: 108 (“ISVD108” in FIG. 10). Concentration of the different molecules (marked as 1-4 in FIG. 10) in blood over time is shown in FIG. 10.

FIGS. 11A-11C. HPLC/SEC analysis of Crypto/PEG conjugates onto molecules with SEQ ID NO.: 107 (FIG. 11A) and SEQ ID NO.: 113 (FIG. 11B and FIG. 11C). Samples: 5 μl injection; Column: TSK-GEL SW mAb HTP (Tosoh Bioscience)-4 μm-4.6×150 mm; Solvent: Isocratic with 100% SEC Buffer Kpi iPrOH 20% pH=7 (Composition: KCl 1 M, 200 mL/KH₂PO₄ 1 M, 52 mL/K₂HPO₄ 1 M, 107 mL/MQ, 441 mL/iPrOH, 200 mL); Run: 15 min at 0.2 mL/min; V₀=Exclusion volume, Vₜ=total volume of the column.

FIG. 12. Schematic representation of the cell-internalization assay used in Examples 11-14.

FIG. 13. Internalization of ALB-3C_K27m_w1 (SEQ ID NO.: 173) and ALB-5C_K27m_w1 (SEQ ID NO.: 174) molecules conjugated to GE11 peptide vs. alanine conjugation on NCI-H226 cells using HSA-pHAb, as described in Example 11.

FIG. 14. Preparation of tert-butyl (2S)-5-[2-(tert-butoxycarbonylamino)ethylamino]-2-(9H-fluoren-9-ylmethoxycarbonylamino)-5-oxo-pentanoate (intermediate 1).

FIG. 15. Preparation of tert-butyl (2S)-2-amino-5-[2-(tert-butoxycarbonylamino)ethylamino]-5-oxo-pentanoate (intermediate 2).

FIG. 16. Preparation of (2S)-5-(2-aminoethylamino)-2-[[4-[(2-amino-4-hydroxy-pteridin-6-yl)methylamino]benzoyl]amino]-5-oxo-pentanoic acid (intermediate 3).

FIG. 17. Preparation of (2S)-2-[[4-[(2-amino-4-oxo-1H-pteridin-6-yl)methylamino]benzoyl]amino]-5-[2-[3-(2,5-dioxopyrrol-1-yl)propanoylamino]ethylamino]-5-oxo-pentanoic acid (folate-aminoethyl-maleimide).

FIG. 18. Internalization of Hela cell lines by the protein-based carrier molecules conjugated with folate. Protein-based carrier building blocks comprised in T028100070 and T028100075 have been conjugated either to maleimide-folate or to maleimide-Ala (negative control), as described in Example 12.

FIGS. 19A and 19B. Schema of the synthesis of the bisM6P ([(2S,3R,4R,5R)-6-[(3S,4S,5S,6R)-2-[[(2R,3S,4S,5S)-6-[[(2S,3S,4R,5R,6S)-4-[(3R,4R,5R,6S)-4,5-dihydroxy-6-(hydroxymethyl)-3-[(3R,4R,5R,6S)-3,4,5-trihydroxy-6-(phosphonooxymethyl)tetrahydropyran-2-yl]oxy-tetrahydropyran-2-yl]oxy-6-[4-[2-[3-[2-[2-[2-[2-(2,5-dioxopyrrol-1-yl)ethoxy]ethoxy]ethoxy]ethoxy]propanoyl]hydrazine]-4-oxo-butoxy]-3,5-dihydroxy-tetrahydropyran-2-yl]methoxy]-3,4,5-trihydroxy-tetrahydropyran-2-yl]methoxy]-4,5-dihydroxy-6-(hydroxymethyl)tetrahydropyran-3-yl]oxy-3,4,5-trihydroxy-tetrahydropyran-2-yl]methyl dihydrogen phosphate).

FIG. 20. Schematic representation of the cell-binding assay used in Examples 13 and 16.

FIG. 21. Binding of T028100069 (SEQ ID NO.: 107), T028100070 (SEQ ID NO.: 108) and T028100075 (SEQ ID NO.: 176) molecules conjugated with bis-M6P (diM6P) vs. Alanine on K-562 cells in Flow Cytometry using HSA-biotin and Streptavidin-Phycoerythrin detection, as described in Example 13. An ISVD specifically binding M6PR (“ISVD specific for M6PR”) was used as positive control, and an ISVD which does not specifically bind M6PR (“ISVD no specific binding to M6PR”) was used as a further negative control.

FIG. 22. Internalization of T028100069 (SEQ ID NO.: 107), T028100070 (SEQ ID NO.: 108) and T028100075 (SEQ ID NO.: 176) CARGO conjugated with bis-M6P vs. Alanine on K-562 cells using HSA-pHAb for detection, as described in Example 13. An ISVD specifically binding M6PR (“ISVD specific for M6PR”) was used as positive control, and an ISVD which does not specifically bind M6PR (“ISVD no specific binding to M6PR”) was used as a further negative control.

FIG. 23. Lipidation QC via mass spec analysis of the DOL=0 (mal-ALA) and DOL 6 preparations confirms the conjugations for both batches. A near full conjugation of undecanoic acid and Alanine was observed.

FIG. 24. Internalization of ALB-RSV_c6 (T028100078, SEQ ID NO.: 113) with 11-Maleimidoundecanoic acid vs Alanine conjugation on BxPC-3 using HSA-pHAb for detection, as described in Example 14.

FIGS. 25A and 25B. (FIG. 25A) SEC analysis of the conjugation of the different mal-PEG molecules on the SH-groups of the side chains of the Cys present in the ISVD-derived building blocks of the different molecules (6×5 kDa, 5×5 kDa, 4×5 kDa and 1×30 kDa). (FIG. 25B) Size and Relative % of the different PEG-conjugated molecules analyzed by SEC.

FIG. 26. Measurement of ISVD-based carrier compound concentrations in rat serum.

FIG. 27: Non-reducing PAGE analysis of a partial CMA1 uploaded CKS-based carrier.

### DEFINITIONS

Unless indicated or defined otherwise, all terms used have their usual meaning in the art, which will be clear to the skilled person. Reference is, for example, made to the standard handbooks, such as Sambrook et al., 1989 (Molecular Cloning: A Laboratory Manual, 2^{nd} Ed., Vols. 1-3, Cold Spring Harbor Laboratory Press), Ausubel et al., 1987 (Current protocols in molecular biology, Green Publishing and Wiley Interscience, New York), Lewin 1985 (Genes II, John Wiley & Sons, New York, N.Y.), Old et al., 1981 (Principles of Gene Manipulation: An Introduction to Genetic Engineering, 2^{nd} Ed., University of California Press, Berkeley, CA), Roitt et al., 2001 (Immunology, 6^{th} Ed., Mosby/Elsevier, Edinburgh), Roitt et al., 2001 (Roitt's Essential Immunology, 10^{th} Ed., Blackwell Publishing, UK), and Janeway et al., 2005 (Immunobiology, 6^{th} Ed., Garland Science Publishing/Churchill Livingstone, New York), as well as to the general background art cited herein.

Unless indicated otherwise, all methods, steps, techniques and manipulations that are not specifically described in detail herein can be performed and have been performed in a manner known per se, as will be clear to the skilled person. Reference is, for example, again made to the standard handbooks and the general background art mentioned herein and to the further references cited therein; as well as to for example the following reviews: Presta 2006 (Adv. Drug Deliv. Rev., 58:640), Levin and Weiss 2006 (Mol. Biosyst., 2:49), Irving et al., 2001 (J. Immunol. Methods, 248:31), Schmitz et al., 2000 (Placenta 21 Suppl. A: S106), Gonzales et al., 2005 (Tumour Biol., 26:31), which describe techniques for protein engineering, such as affinity maturation and other techniques for improving the specificity and other desired properties of proteins such as immunoglobulins.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the technology described herein. Such equivalents are intended to be encompassed by the present technology.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

The term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acids or nucleotide sequences encoding the same, unless the context requires a more limited interpretation. Amino acid sequences are interpreted to mean a single amino acid or an unbranched sequence of two or more amino acids, depending on the context. Nucleotide sequences are interpreted to mean an unbranched sequence of 3 or more nucleotides.

It is understood that any reference to the amino acid sequences is meant to encompass post-translational modifications of these sequences occurring in mammalian cells such as CHO cells, including, but not limited to, N-glycosylation, O-glycosylation, deamidation, Asp isomerization/fragmentation, pyro-glutamate formation, removal of C-terminal lysine, and Met/Trp oxidation.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein).

Amino acids are organic compounds that contain amino[a] (—NH⁺₃) and carboxylate (—CO⁻₂) functional groups, along with a side chain (R group) specific to each amino acid. For instance, amino acids include those L-amino acids commonly found in naturally occurring proteins. “Amino acids”, in the context of the present technology, also include D-amino acids and non-natural, unusual or unnatural amino acids, as described below. Amino acid residues will be indicated according to the standard three-letter or one-letter amino acid code. Reference is made to Table A-2 on page 48 of WO 08/020079. Examples of amino acids commonly found in proteins and represented in the genetic code are listed in Table 1 below. Other common amino acids (excluding those listed in Table 1 below) are described on the table on p. 624 of Pure & Appl. Chem., Vol. 56, No. 5, pp. 595-624, 1984, reproduced below as Table 2 for convenience.

TABLE 1
Common amino acids (IUPAC)

| 1-Letter | 3-Letter | |
|---|---|---|
| Code | Code | Name |
| A | Ala | Alanine |
| B | Asx | Aspartic acid or |
| | | Asparagine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Uncommon or |
| | | Unspecified |
| Y | Tyr | Tyrosine |
| Z | Glx | Glutamic acid or |
| | | Glutamine |

TABLE 2
Further amino acids

| | | Structure of substance or of derived ion in the form |
|---|---|---|
| Trivial name | Symbol | predominating at neutral pH |
| β-Alanine | βAla | NH [symbol] —CH [symbol] —COO⁻ |
| Allysine | — | HCO—[CH [symbol] ]₃—CH(NH₃⁺)COO⁻ |
| Citrulline | Cit | NH₃—CO—NH—[CH [symbol] ] [symbol] —CH(NH₃⁺)COO⁻ |
| Cystathionine | 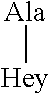 | 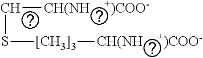 |
| Cystoic acid | Cya | ⁻O₃S—CH₂—CH(NH₃⁺)COO⁻ |
| Cystine | 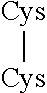 | 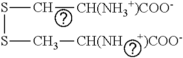 |
| Dopa | — | 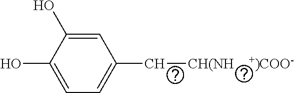 |
| Homocysteine | Hcy | HS—CH₂—CH₃—CH(NH₃⁺)COO⁻ |
| Homoserine | Hse | HO CH₂ CH [symbol] —CH(NH₃⁺)COO⁻ |
| Homoserine lactone | Hsl | 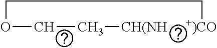 |
| Lanthionine | 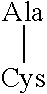 | 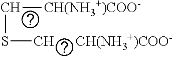 |
| Ornithine | Orn | NH₃⁺—[CH₂]₃—CH(NH₃⁺)COO⁻ |
| 5-Oxoptoline | Glp | 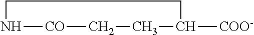 |
| Sarcosine | Sar | CH [symbol] —NH₃⁺—CH₂—COO⁻ |
| Thyronine | — | 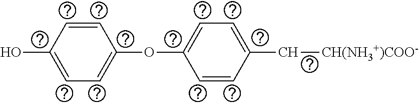 |
| Thyroxine | Thx | 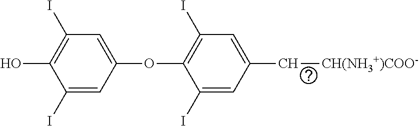 |

[symbol]

indicates data missing or illegible when filed

D-amino acids are also encompassed by the definition of “amino acid”. As used herein, the term “D-amino acid” refers to amino acids where the stereogenic carbon alpha to the amino group has the D-configuration.

Unusual, unnatural or non-natural amino acids are also encompassed by the definition of “amino acid”. As used herein, the term “unnatural amino acid” or “non-canonical amino acid” or “non-natural amino acid” or “novel amino acid” (or the like) refers to an amino acid that is not one of the twenty amino acids commonly found in peptides synthesized in nature, and known by the one letter abbreviations A, R, N, C, D, Q, E, G, H, I, L, K, M, F, P, S, T, W, Y and V. Exemplary unnatural amino acids are described in Young et al., “Beyond the canonical 20 amino acids: expanding the genetic lexicon,” J. of Biological Chemistry, 285 (15): 11039-11044 (2010), the disclosure of which is incorporated herein by reference.

Alexander R. Nödling et al. (“Using genetically incorporated unnatural amino acids to control protein functions in mammalian cells”, Essays Biochem, 3 Jul. 2019; 63 (2): 237-266), the disclosure of which is incorporated herein by reference, provides an overview of unnatural amino acids that have been successfully incorporated into proteins in mammalian cells, see, e.g., Table 1 starting on p. 240.

Non-limiting examples of unnatural amino acids include: p-acetyl-phenylalanine, O-4-allyl-L-tyrosine, 4-propyl-L-tyrosine, L-Dopa, p-azido-phenylalanine, N6-(propargyloxy)-carbonyl-L-lysine (PrK), azido-lysine (N6-azidoethoxy-carbonyl-L-lysine, AzK). In some embodiments, the unnatural amino acid comprises a selective reactive group, or a reactive group for site-selective labeling or conjugation of a moiety or cargo. In some instances, the chemistry is a biorthogonal reaction (e.g., biocompatible and selective reactions). In some cases, the chemistry is a Cu (I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. For further examples of unnatural amino acids, we refer to WO 2021/072167, the disclosure of which is incorporated herein by reference.

The terms “protein”, “peptide”, “protein/peptide”, and “polypeptide” are used interchangeably throughout the present disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide and a protein; that polypeptides may be made by chemical synthesis or recombinant methods; and that proteins are generally made in vitro or in vivo by recombinant methods as known in the art.

By convention, the amide bond in the primary structure of polypeptides is in the order that the amino acids are written, in which the amine end (N-terminus) of a polypeptide is always on the left, while the acid end (C-terminus) is on the right.

Any amino acid sequence that contains post-translationally modified amino acids may be described as the amino acid sequence that is initially translated using the symbols shown in Table 1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

In the context of the present technology, the terms “specificity”, “binding specifically” or “specific binding” refer to the number of different target molecules, such as antigens, to which a particular binding unit can bind with sufficiently high affinity (see below). “Specificity”, “binding specifically” or “specific binding” are used interchangeably herein with “selectivity”, “binding selectively” or “selective binding”. Generally, binding units, such as binding ISVDs, specifically bind to their designated targets.

The specificity/selectivity of a binding unit can be determined based on affinity. The affinity denotes the strength or stability of a molecular interaction. The affinity is commonly given by the K_{D}, or dissociation constant, which has units of mol/litre (or M). The affinity can also be expressed as an association constant, K_{A}, which equals 1/K_{D} and has units of (mol/litre)⁻¹ (or M⁻¹).

The affinity is a measure for the binding strength between a moiety and a binding site on a target molecule: the lower the value of the K_{D}, the stronger the binding strength between a target molecule and a targeting moiety.

The K_{D}-value characterizes the strength of a molecular interaction also in a thermodynamic sense as it is related to the change of free energy (DG) of binding by the well-known relation DG=RT.ln(K_{D}) (equivalently DG=-RT.ln(K_{A})), where R equals the gas constant, T equals the absolute temperature and In denotes the natural logarithm.

The K_{D} may also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1 s⁻¹ (t_{1/2}=0.69 s).

The measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule or molecules. In such situation the measured affinity may be affected by the avidity of the interaction by the two molecules.

The dissociation constant (K_{D}) may be the actual or apparent dissociation constant, as will be clear to the skilled person. Methods for determining the K_{D} will be clear to the skilled person, and for example include the techniques mentioned below. In this respect, it will also be clear that it may not be possible to measure dissociation constants of more than 10⁻⁴ moles/litre or 10⁻³ moles/litre (e.g., of 10⁻² moles/litre). Optionally, as will also be clear to the skilled person, the (actual or apparent) K_{D} may be calculated on the basis of the (actual or apparent) association constant (K_{A}), by means of the relationship (K_{D}=1/K_{A}). K_{A}=1/K_{D}→K_{A}=[AB]/[A]·[B].

The term “about” used in the context of the parameters or parameter ranges of the provided herein shall have the following meanings. Unless indicated otherwise, where the term “about” is applied to a particular value or to a range, the value or range is interpreted as being as accurate as the method used to measure it. If no error margins are specified in the application, the last decimal place of a numerical value indicates its degree of accuracy. Where no other error margins are given, the maximum margin is ascertained by applying the rounding-off convention to the last decimal place, e.g., for a pH value of about pH 2.7, the error margin is 2.65-2.74. However, for the following parameters, the specific margins shall apply: a temperature specified in ° C. with no decimal place shall have an error margin of +1° C. (e.g., a temperature value of about 50° C. means 50° C.±1° C.); a time indicated in hours shall have an error margin of 0.1 hours irrespective of the decimal places (e.g., a time value of about 1.0 hours means 1.0 hours±0.1 hours; a time value of about 0.5 hours means 0.5 hours±0.1 hours).

In the present application, any parameter indicated with the term “about” is also contemplated as being disclosed without the term “about”. In other words, embodiments referring to a parameter value using the term “about” shall also describe an embodiment directed to the numerical value of said parameter as such. For example, an embodiment specifying a pH of “about pH 2.7” shall also disclose an embodiment specifying a pH of “pH 2.7” as such; an embodiment specifying a pH range of “between about pH 2.7 and about pH 2.1” shall also describe an embodiment specifying a pH range of “between pH 2.7 and pH 2.1”, etc.

For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position). Alternatively, the degree of sequence identity between two or more nucleotide sequences may be calculated using a known computer algorithm for sequence alignment such as NCBI Blast v2.0, using standard settings. Some other techniques, computer algorithms and settings for determining the degree of sequence identity are for example described in WO 04/037999, EP 0967284, EP 1085089, WO 00/55318, WO 00/78972, WO 98/49185 and GB 2357768. Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the 3D structure, function, activity, or other biological properties of the polypeptide. Such conservative amino acid substitutions are well known in the art, for example from WO 04/037999, GB 335768, WO 98/49185, WO 00/46383, and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Amino acid sequences and nucleic acid sequences are said to be “exactly the same” if they have 100% sequence identity (as defined herein) over their entire length. When comparing two amino acid sequences, the term “amino acid difference” refers to an insertion, deletion or substitution of a single amino acid residue on a position of the first sequence, compared to the second sequence; it being understood that two amino acid sequences may contain one, two or more such amino acid differences.

According to the present description, “protein solubility” is a thermodynamic parameter defined as the concentration of protein in a saturated solution that is in equilibrium with a solid phase, either crystalline or amorphous, under a given set of conditions (see, e.g., Kramer RM. et al., “Toward a molecular understanding of protein solubility: increased negative surface charge correlates with increased solubility”, Biophys J., 2012, 102 (8): 1907-15).

### Protein-Based Building Block

The molecule of the present technology comprises, or alternatively, consists of, at least one protein-based carrier building block (also referred herein as “carrier building block”, “protein-based building block”, or simply “building block” or “carrier”), as defined herein. For instance, the molecule of the present technology may comprise or, alternatively, consist of, a single protein-based carrier building block. In other embodiments, the molecule comprises more than one protein-based building blocks, such as two, three, four, five, six or more carrier building blocks. The protein-based carrier building block comprises (and, preferably, consists of) at least part of a protein or a whole structured protein, i.e., the protein-based carrier building block is preferably a polypeptide.

The protein-based carrier building block is designed as a “carrier” or “delivery” moiety, with at least one attachment point or conjugation site, preferably with at least two attachment points or conjugation sites, for conjugation or attachment of cargos, as defined in detail below. Suitable cargos include proteins, peptides, toxic payloads, nucleic acids, oligonucleotides, fluorophores, glycans, chelators for/and radio-isotopes, polyethylene glycol (PEG) molecules, vitamins (such as biotin or folate), etc. Specific non-limiting examples of suitable cargos are depicted below in the present description.

An attachment point or conjugation site, in the context of the present technology, refers to any group comprised in the protein-based building block which is suitable for attaching or conjugating a cargo to it. The attachment point or conjugation site is preferably present at a solvent-accessible position in the protein-based building block, as explained in detail below. An attachment point or conjugation site may be a reactive group present in the side chain of any amino acid in the protein-based carrier building block, preferably an amino acid present at a solvent-accessible position in the protein-based carrier building block, or may be the N-terminal primary amine, and/or the C-terminal carboxylic group of the protein-based building block. The attachment point/conjugation site allows the formation of a covalent bond with a group present in the cargo to be conjugated and/or attached to the protein-based carrier building block. In a preferred embodiment, the attachment point or conjugation site is a reactive group present in the side chain of an amino acid in the protein-based carrier building block, preferably present at a solvent-accessible position in the protein-based carrier building block, which allows the formation of a covalent bond with a group present in the cargo to be conjugated and/or attached to the protein-based carrier building block. In another embodiment, two of the conjugation sites or attachment points of the protein-based building block are reactive groups present in the side chain of two amino acids present in the protein-based carrier building block, preferably two amino acid presents at solvent-accessible positions in the protein-based carrier building block. In another embodiment, all of the conjugation sites or attachment points of the protein-based building block are reactive groups present in the side chain of amino acids present in the protein-based carrier building block, preferably amino acid presents at solvent-accessible positions in the protein-based carrier building block.

### Globular Three Dimensional (3D) Structure

The protein-based carrier building block of the present technology has a globular three-dimensional (3D) structure, i.e., it is or comprises a structured protein with a globular 3D structure. Globular proteins have approximately spherical shape. Nearly all globular proteins contain substantial numbers of a-helices and/or B-sheets folded into a compact structure that is stabilized by both polar and nonpolar interactions. The globular 3D structure forms naturally and often involves interactions mediated by the side chains of the amino acids. Most often, the hydrophobic amino acid side chains are buried, closely packed, in the interior of a globular protein, out of contact with water. Hydrophilic amino acid side chains lie on the surface of the globular proteins exposed to the water. Consequently, globular proteins are usually very soluble in aqueous solutions (from “Gene Expression: Translation of the Genetic Code”, Chang-Hui Shen, in Diagnostic Molecular Biology, 2019). In the context of the present technology, a protein or part of a protein with globular 3D structure can be defined as a protein or part of it which comprises at least one a-helix and/or at least one B-sheet as part of its secondary structure. From a simple sequence of amino acids to its final 3D structure, a protein passes through four levels of structuring known as primary, secondary, tertiary, and quaternary. At the end of these stages the protein begins to fold up into a stable 3D structure that will allow it to fulfil its proper function. Hence, the amino acid sequence of a protein is known as the “primary structure” of that protein. The “secondary structure” can be defined as the arrangement of a polypeptide chain into more or less regular hydrogen-bonded structures, and it has two basic elements:
Alpha helix—spiral configuration of a polypeptide chain with 3.6 residues (amino acids) per turn. The helix may be left-handed or right-handed, and the latter is more common.
Beta strand (or beta-sheet)—two adjacent polypeptide strands that are bonded together. Two or more strands may interact to form a beta sheet.

Finally, the “tertiary structure” can be defined as the level of protein structure at which an entire polypeptide chain has folded into a 3D structure. In multi-chain proteins, the term tertiary structure applies to the individual chains. See Smith, A. D., et al., eds. 1997, Oxford Dictionary of Biochemistry and Molecular Biology, New York: Oxford University Press.

The three-dimensional structure of a protein can be determined by techniques such as X-ray crystallography, nuclear magnetic resonance (NMR), cryo-electron microscopy (EM) or circular dichroism (CD). X-ray crystallography is a common technique used to determine 3D protein structure, but also NMR (suited for small proteins) and cryo-EM (suited for large proteins) can provide information about a protein's tertiary structure. Circular dichroism is an excellent method for rapidly evaluating the secondary structure, folding and binding properties of proteins, see, e.g., Jones, C. (“Circular dichroism of biopharmaceutical proteins in a quality-regulated environment”, J Pharm Biomed Anal., 2022, 219:114945). Because the CD spectra of proteins are so dependent on their conformation, CD can be used to estimate the structure of unknown proteins and monitor conformational changes due to temperature, mutations, heat, denaturants or binding interactions. For instance, a-helical proteins have negative bands at 222 nm and 208 nm and a positive band at 193 nm. Proteins with well-defined antiparallel β-pleated sheets (β-helices) have negative bands at 218 nm and positive bands at 195 nm, while disordered proteins have very low ellipticity above 210 nm and negative bands near 195 nm. See Greenfield NJ., “Using circular dichroism spectra to estimate protein secondary structure”, Nat Protoc., 2006, 1 (6): 2876-90 for further details.

Hence, the protein-based carrier building block of the present technology comprises at least one α-helix and/or at least one β-sheet as part of its secondary structure, preferably more than one α-helix and/or more than one β-sheet as part of its secondary structure, leading to a globular 3D tertiary structure. This allows the engineering of site- and stereospecific-conjugation sites or attachment points, as described in detail in this specification. The presence of at least one α-helix and/or at least one β-sheet in a certain polypeptide or protein can be determined by known techniques, as explained above, such as, e.g., CD.

### Solubility

The protein-based carrier building block of the present technology is soluble. In the context of the present technology, a soluble building block means that the building block has a solubility of 10 mg/ml or more, preferably of 20 mg/mL, preferably of 50 mg/ml or more, and even more preferably of 100 mg/ml or more, measured in water or a suitable buffer or solvent (e.g., an aqueous solution, or a physiological buffer, such as a buffer which is amenable for parenteral administration) at room temperature (RT). In a preferred embodiment, the solubility of the protein-based carrier building block is measured in water or in a suitable buffer at RT, more preferably in a buffer such as citrate buffer (e.g., citrate buffer 5 mM) or PBS, at pH 7.0 or 7.4, at RT. Other preferred suitable buffers which are suitable for measuring the solubility of the protein-based carrier building block are Dulbecco's phosphate buffered saline (DPBS, which is a balanced salt solution containing potassium chloride, monobasic potassium phosphate, sodium chloride, and dibasic sodium phosphate, e.g., 2.7 mM KCl, 1.5 mM KH₂PO₄, 136.9 mM NaCl, 8.9 mM Na₂HPO₄·7H₂O, pH7.0-7.3, commercially available from GIBCO (Nr14190-094)), preferably pH 7.0 or 7.3 or 7.4, at RT, or histidine buffer at pH 6.5, at RT (comprising histidine (10 mM to 100 mM, such as 10 mM), sucrose (1% to 10%, such as 10%) and, optionally, Tween 80 (0.001% to 1%, such as 0.01%)), or phosphate buffer pH 7.0, at RT (comprising NaH₂PO₄/Na₂HPO₄ (10 and 50 mM, such as 10 mM), sodium chloride (NaCl) (100-150 mM, such as 130 mM NaCl) and, optionally, Tween 80 (0.001% to 1%, such as 0.01%)).

The skilled person is aware of methods to measure the solubility of a protein solution. For instance, the supplementary material of Kramer R M. et al., “Toward a molecular understanding of protein solubility: increased negative surface charge correlates with increased solubility”, Biophys J., 2012, 102 (8): 1907-15) describes solubility measurements of folded proteins.

Additionally or alternatively, solubility measurements can be performed as follows. The protein solution (e.g., in citrate buffer 5 mM, pH 7.0, or in PBS pH 7.4, or in water, or in any of the suitable buffers described above) is concentrated by ultrafiltration (e.g., via tangential flow filtration (TFF)) until some cloudiness appears in the solution. Then, the solution is spined at high speed or 0.22 μm filtered to remove any non-soluble material, and the OD₂₈₀ of the supernatant is measured. Using the molar extinction coefficient of the specific protein, the protein concentration of the supernatant (and, thus, the concentration of the protein in a saturated solution that is in equilibrium with a solid phase, i.e., the protein solubility) is obtained.

For instance, in the context of the present technology, physiological buffers suitable for parenteral administration can include the following components: Glutamate, Tartrate, Lactate, Citrate, Malate, Gluconate, Ascorbate, Maleate, Phosphate, Succinate, Acetate, Bicarbonate, Aspartate, Histidine, Benzoate, Tromethamine, Diethanolamine, Ammonium or Glycine. The most common buffers used in parenteral formulations are based on histidine, citrate, phosphate, and acetate (see, e.g., Broadhead J, Gibson M., “Parenteral dosage forms”, in: Gibson M., editor, “Pharmaceutical preformulation and formulation”, New York: Informa healthcare; 2009, p. 325-47).

Preferably, the protein-based carrier building block of the present technology is soluble in reduced state, i.e., it is soluble when the —SH groups (e.g., in the side chain of one or more Cys) present at solvent accessible positions in its amino acid sequence, if any, is (are) in a reduced form (as “—SH”), and not oxidized. For instance, a protein-based carrier building block may be reduced when subjected to reducing conditions for enough time. For instance, reducing conditions may mean using beta-mercaptoethanol (2-ME), dithiothreitol (DTT) or and TCEP (Tris (2-carboxyethyl) phosphine).

Size (molecular mass) The protein-based carrier building block of the present technology has a size (molecular mass) of about 2.5 to about 70 kDa, such as about 2.5, 5, 10, 15, 20, 25, 30, 35, 40, 50, 60, 65 or about 70 kDa. Preferably, the building block is a small building block with a size of about 2.5 to about 50 kDa, such as of about 2.5 to about less than 50 kDa, such as about 2.5 to about 40 kDa, or about 2.5 to about 35 kDa, more preferably of about 2.5 to about 30 kDa, such as about 5 to about 30 kDa, or about 7 to about 30 kDa, or about 10 to about 30 kDa, or about 2.5 to about 25 kDa, or about 5 to about 25 kDa, or about 7 to about 25 kDa, or about 10 to about 25 kDa, or about 2.5 to about 20 kDa, or about 5 to about 20 kDa, or about 7 to about 20 kDa, or about 10 to about 20 kDa, or about 2.5 to about 18 kDa, or about 5 to about 18 kDa, or about 7 to about 18 kDa, or about 10 to about 18 kDa. More preferably, the building block of the present technology has a size of about 2.5 to about 16 kDa, such as about 5 to about 16 kDa, or about 7 to about 16 kDa, or about 10 to about 16 kDa, or such as about 2.5, 3, 5, 6.5, 7, 10, 11, 12, 13, 14, 15 or 16 kDa. For instance, the protein-based building block may have a size (molecular mass) of about 6 kDa, or of about 7 kDa, or of about 15 kDa, or of about 16 kDa. In an even more preferred embodiment, the protein-based carrier building block has a size of about 15 kDa.

### Non-Functionality

The protein-based carrier building block of the present technology does not specifically bind to any human protein. If the building block shows any interaction with one or more human proteins, such interaction is characterized by low specificity and/or low affinity, as defined herein.

For instance, the protein-based carrier building block of the present technology does not specifically bind crystallizable fragment (Fc) receptors (FcRs), Fc-binding proteins or Fc-sensors. For instance, the protein-based carrier building block does not specifically bind C-type lectin receptors (CLRs). All antibodies possess two functional domains—one that confers antigen specificity, known as the antigen-binding fragment (Fab), and another that drives antibody function, known as the crystallizable fragment (Fc). The specific effector functions that are triggered by antibodies are determined by the receptors to which the antibody Fc domain binds and the specific innate immune cells on which these FcRs are expressed. These sensors include both classical FcRs and non-classical C-type lectin receptors (CLRs), see Lu, L. et al., “Beyond binding: antibody effector functions in infectious diseases”, Nat Rev Immunol, 2018, 18, 46-61. Table 1 of Lu, L. et al provides non-limiting examples of Fc domain sensors (e.g., Fcγ or FcRn) to which the protein-based carrier building block of the present technology do not specifically bind. Consequently, the protein-based carrier building block of the present technology does not show effector functions of conventional antibodies mediated by the Fc domain. In another embodiment, the protein-based carrier building block and/or the molecule does not specifically bind crystallizable fragment (Fc) receptors (FcRs), Fc-binding proteins or Fc-sensors. For instance, the protein-based carrier building block and/or the molecule does not specifically bind C-type lectin receptors (CLRs). Hence, in one embodiment, none of the components comprised in the molecule of the present technology (e.g., at least one protein-based carrier building block and/or at least one cargo attached or conjugated to it) specifically bind crystallizable fragment (Fc) receptors (FcRs), Fc-binding proteins, Fc-sensors and/or CLRs. In another embodiment, the protein-based building block and/or the molecule of the present technology does not show effector functions of conventional antibodies mediated by the Fc domain, i.e., none of the components comprised in the molecule of the present technology show effector functions of conventional antibodies mediated by the Fc domain. In one embodiment, the molecule of the present technology does not include conventional V_{H}-V_{L} pairing/interaction and/or does not include C_{L}-C_{H}1 pairing such as C_{L}-C_{H}1 binding disulphide bridges.

In another embodiment, the protein-based carrier building block of the present technology does not specifically bind the variable domain of the light chain (V_{L}) and/or the variable domain of the heavy chain (V_{H}) of an antibody, such as the V_{L} and/or the V_{H} of a monoclonal antibody (mAb). In another embodiment, the protein-based carrier building block does not specifically bind the first constant domain of the heavy chain (C_{H}1) of an antibody, such as the C_{H}1 of a mAb. In another embodiment, the protein-based carrier building block does not specifically bind the constant domain of the light chain (C_{L}) of an antibody, such as the C_{L} of a mAb. In another embodiment, the protein-based carrier building block does not specifically bind the third constant domain of the heavy chain (C_{H}3) of an antibody, such as the C_{H}3 of a mAb. In another embodiment, the protein-based carrier building block does not specifically bind the second constant domain of the heavy chain (C_{H}2) of an antibody, such as the C_{H}2 of a mAb. In one embodiment, the molecule and/or the building block of the present technology is not a Fab fragment from an antibody, such as from a mAb. In one embodiment, the molecule and/or the building block of the present technology is not a C_{H}, preferably is not a C_{H}1 fragment from an antibody, such as from a mAb. The molecule and/or the building block of the present technology is not an antibody, such as a mAb, is not a Fc fragment, or a Fv fragment.

The protein-based carrier building block of the present technology may derive from a target-binding protein (such as an ISVD, a DARPin, an affibody or an affitin) (the “protein-based carrier building block precursor”). In the context of the present technology, a “protein-based carrier building block precursor” or “building block precursor” is a protein-based moiety which may be modified to generate the protein-based carrier building block comprised in the molecule of the present technology.

In the context of the present technology, the “protein-based carrier building block precursor” is a protein which is modified (e.g., by point mutations and/or by addition/deletion of amino acids to its sequence) to generate the protein-based carrier building block of the present technology. For instance, the “protein-based carrier building block precursor” is modified so that it no longer specifically binds any human protein, preferably so that it also does not specifically bind any (non-human) molecule (including non-human biomolecule) and/or any non-protein (human) molecule (including biomolecule), in particular any molecule (including biomolecule) to which the precursor specifically binds. In addition, if necessary, the “protein-based carrier building block precursor” is modified so that it incorporates one or more attachment points or conjugation sites as described herein. The “protein-based carrier building block precursor” has a sequence identity of at least 60%, such as at least 70%, or at least 75%, preferably of at least 80% with the protein-based carrier building block derived from it. For instance, the “protein-based carrier building block precursor” has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with the protein-based carrier building block derived from it. For instance, the “protein-based carrier building block precursor” may share the whole amino acid sequence with protein-based carrier building block derived from it with the exception of at least one, such as one, two, three, four, five, six, seven, eight, nine, ten, eleven, twelve, thirteen, fourteen, fifteen, twenty or more amino acids. Of course, the protein-based carrier building block derived from a protein-based carrier building block precursor has a globular 3D structure, is soluble, has a size (molecular mass) of about 2.5 to about 70 kDa, such as about 2.5 to about 50 kDa, such as of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, does not specifically bind any human protein and preferably does not specifically bind any protein or non-protein molecule to which the precursor specifically binds.

Preferably, the carrier building block of the present technology does also not specifically bind to any non-protein molecule (including non-protein biomolecules, such as nucleic acids, e.g., DNA and/or RNA, lipids (e.g., phosphatidylserine (PS)) or glycans), e.g., to any non-protein human molecule (including biomolecule), such as human nucleic acids, e.g., human DNA and/or human RNA, human lipids (e.g., such as phosphatidylserine (PS)) or human glycans, e.g., human glycoplipids. In particular, preferably, the carrier building block does also not specifically bind to any non-protein molecule (including biomolecules) (such as nucleic acids such as DNA and/or RNA, lipids (e.g., such as phosphatidylserine (PS)) or glycans), e.g., to any non-protein human molecule (including biomolecules), such as human nucleic acids, e.g., human DNA and/or human RNA, human lipids (e.g., phosphatidylserine (PS)) or human glycans, e.g., human glycoplipids to which the protein-based carrier building block precursor specifically binds (i.e., the protein-based building block preferably does also not specifically bind to the precursor's target, e.g., a non-protein molecule (including biomolecules) or a non-human protein).

In another preferred embodiment, the protein-based building block of the present technology does also not specifically bind to any (non-human) molecule (including biomolecules) which the protein-based carrier building block precursor specifically binds to (i.e., the protein-based building block preferably does also not specifically bind to the precursor's target, e.g., a non-human protein or a non-protein molecule (including biomolecules)), or binds to any (non-human) molecule which the protein-based carrier building block precursor specifically binds to (i.e., the protein-based building block preferably does also not specifically bind to the precursor's target, e.g., a non-human protein or a non-protein molecule) with a K_{D} value greater than 5×10⁻⁴ mol/litre, as described herein. For example, if the precursor of the protein-based carrier building block is an anti-RSV (respiratory syncytial virus) ISVD (i.e., it specifically binds one or more proteins of RSV, such as protein F of RSV), the protein-based carrier building block derived from it preferably does not specifically bind those RSV proteins (or binds those proteins, such as protein F of RSV preferably with a K_{D} value greater than 5×10⁻⁴ mol/litre, as described herein).

For example, if the precursor of the protein-based carrier building block specifically binds to virus (e.g., it is an anti-viral ISVD, an anti-viral DARPin, an anti-viral affitin, an anti-viral affibody, or the like) and/or to viral molecules (e.g., it specifically binds one or more viral biomolecules, e.g., viral proteins, viral nucleic acids, viral lipids or viral glycans), the protein-based carrier building block derived from it preferably does not specifically bind those virus and/or viral molecules (or binds those virus and/or viral molecules preferably with a K_{D} value greater than 5×10⁻⁴ mol/litre, as described herein). In other embodiments, the protein-based carrier building block specifically binds to virus (e.g., it is an anti-viral ISVD, an anti-viral DARPin, an anti-viral affitin, an anti-viral affibody, or the like) and/or to viral molecules (e.g., it specifically binds one or more viral biomolecules, e.g., viral proteins, viral nucleic acids, viral lipids or viral glycans), as its precursor does, but the specific binding is eliminated when at least a cargo is attached to the protein-based building block. Example of viruses which the protein-based building block precursor (and/or protein-based building block of the present technology) may specifically bind are the following: RSV, influenza virus, rabies virus, potyvirus, bacteriophage, rotavirus, HIV protein, Hepatitis B virus, Hepatitis C virus, norovirus, Shiga toxins from lambdoid prophages, Herpes simplex virus, Grapevine fanleaf virus (GFLV), Ebola, Middle East respiratory syndrome (MERS) virus, acute respiratory syndrome (SARS) virus, SARS-COV2, Vibrio or a White Spot Syndrome virus, cytomegalovirus, parvovirus, ZIKA virus, Chikungunya Virus (CHIKV). Hence, in one embodiment, the protein-based building block precursor, e.g., an ISVD, may specifically bind to one or more of these viruses (or molecules, including biomolecules, comprised therein). The resulting protein-based building block may not specifically bind to the virus (or molecules, including biomolecules, comprised therein) to which the precursor binds. If the protein-based building block of the present technology shows specific binding towards one or more of these viruses (or molecules, including biomolecules, comprised therein), that specific binding as described herein is lost when at least a cargo is attached to the at least one conjugation site comprised therein.

For example, if the precursor of the protein-based carrier building block specifically binds to protozoa (a microorganism, unicellular eukaryote) (e.g., it is an anti-protozoa ISVD, an anti-protozoa DARPin, an anti-protozoa affitin, an anti-protozoa affibody, or the like) and/or to protozoa molecules (e.g., it specifically binds one or more protozoa biomolecules, e.g., protozoa proteins, protozoa nucleic acids, protozoa lipids or protozoa glycans), the protein-based carrier building block derived from it preferably does not specifically bind those protozoa and/or protozoa molecules (or binds those protozoa and/or protozoa molecules preferably with a K_{D} value greater than 5×10⁻⁴ mol/litre, as described herein). In other embodiments, the protein-based carrier building block specifically binds to protozoa (e.g., it is an anti-protozoa ISVD, an anti-protozoa DARPin, an anti-protozoa affitin, an anti-protozoa affibody, or the like) and/or to protozoa molecules (e.g., it specifically binds one or more protozoa biomolecules, e.g., protozoa proteins, protozoa nucleic acids, protozoa lipids or protozoa glycans), as its precursor does, but the specific binding is eliminated when at least a cargo is attached to the protein-based building block. Examples of protozoa and protozoa molecules to which the protein-based building block precursor (and/or protein-based building block of the present technology) may specifically bind are the following: Trypanosoma evansi, Eimeria stiedae, Variant surface glycoprotein (VSG). Hence, in one embodiment, the protein-based building block precursor, e.g., an ISVD, may specifically bind to one or more of these protozoa (or molecules, including biomolecules, comprised therein). The resulting protein-based building block may not specifically bind to the protozoa (or molecules, including biomolecules, comprised therein) to which the precursor binds. If the protein-based building block of the present technology shows specific binding towards one or more of these protozoa (or molecules, including biomolecules, comprised therein), that specific binding as described herein is lost when at least a cargo is attached to the at least one conjugation site comprised therein.

For example, if the precursor of the protein-based carrier building block specifically binds to mammalian proteins (e.g., it is an anti-mammalian protein ISVD, an anti-mammalian protein DARPin, an anti-mammalian protein affitin, an anti-mammalian protein affibody, or the like), the protein-based carrier building block derived from it preferably does not specifically bind those mammalian proteins (or binds those mammalian proteins preferably with a K_{D} value greater than 5×10⁻⁴ mol/litre, as described herein). In other embodiments, the protein-based carrier building block specifically binds to mammalian proteins, as its precursor does, but the specific binding is eliminated when at least a cargo is attached to the protein-based building block. Example of a mammalian protein to which the protein-based building block precursor (and/or protein-based building block of the present technology) may specifically bind is bovine serum albumin. Hence, in one embodiment, the protein-based building block precursor, e.g., an ISVD, may specifically bind to this mammalian protein. The resulting protein-based building block may not specifically bind to the mammalian protein to which the precursor binds. If the protein-based building block of the present technology shows specific binding towards this mammalian protein, that specific binding as described herein is lost when at least a cargo is attached to the at least one conjugation site comprised therein.

For example, if the precursor of the protein-based carrier building block specifically binds to avian proteins (e.g., it is an anti-avian protein ISVD, an anti-avian protein DARPin, an anti-avian protein affitin, an anti-avian protein affibody, or the like), the protein-based carrier building block derived from it preferably does not specifically bind those avian proteins (or binds those avian proteins preferably with a K_{D} value greater than 5×10⁻⁴ mol/litre, as described herein). In other embodiments, the protein-based carrier building block specifically binds to avian proteins, as its precursor does, but the specific binding is eliminated when at least a cargo is attached to the protein-based building block. Example of an avian protein to which the protein-based building block precursor (and/or protein-based building block of the present technology) may specifically bind is Ovalbumin (chicken). Hence, in one embodiment, the protein-based building block precursor, e.g., an ISVD, may specifically bind to this avian protein. The resulting protein-based building block may not specifically bind to the avian protein to which the precursor binds. If the protein-based building block of the present technology shows specific binding towards this avian protein, that specific binding as described herein is lost when at least a cargo is attached to the at least one conjugation site comprised therein.

For example, if the precursor of the protein-based carrier building block specifically binds to yeast and/or moulds proteins (e.g., it is an anti-yeast and/or anti-moulds protein ISVD, an anti-yeast and/or moulds protein DARPin, an anti-yeast and/or moulds protein affitin, an anti-yeast and/or moulds protein affibody, or the like), the protein-based carrier building block derived from it preferably does not specifically bind those yeast and/or moulds proteins (or binds those yeast and/or moulds proteins preferably with a K_{D} value greater than 5×10⁻⁴ mol/litre, as described herein). In other embodiments, the protein-based carrier building block specifically binds to yeast and/or moulds proteins, as its precursor does, but the specific binding is eliminated when at least a cargo is attached to the protein-based building block. Examples of yeast and moulds proteins to which the protein-based building block precursor (and/or protein-based building block of the present technology) may specifically bind are yeast extract, inactivated yeast, Candida. Hence, in one embodiment, the protein-based building block precursor, e.g., an ISVD, may specifically bind to one or more of these yeast and/or moulds proteins. The resulting protein-based building block may not specifically bind to at least one of these yeast and/or moulds proteins to which the precursor binds. If the protein-based building block of the present technology shows specific binding towards these yeast and/or moulds proteins, that specific binding as described herein is lost when at least a cargo is attached to the at least one conjugation site comprised therein.

For example, if the precursor of the protein-based carrier building block specifically binds to plant proteins (e.g., it is an anti-plant protein ISVD, an anti-plant protein DARPin, an anti-plant protein affitin, an anti-plant protein affibody, or the like), the protein-based carrier building block derived from it preferably does not specifically bind those plant proteins (or binds those plant proteins preferably with a K_{D} value greater than 5×10⁻⁴ mol/litre, as described herein). In other embodiments, the protein-based carrier building block specifically binds to plant proteins, as its precursor does, but the specific binding is eliminated when at least a cargo is attached to the protein-based building block. Examples of plant proteins to which the protein-based building block precursor (and/or protein-based building block of the present technology) may specifically bind are Starch Branching Enzyme II (maize), polyphenol, Linoic acid (Sunflower, maize), plant seed. Hence, in one embodiment, the protein-based building block precursor, e.g., an ISVD, may specifically bind to one or more of these plant proteins. The resulting protein-based building block may not specifically bind to at least one of these plant proteins to which the precursor binds. If the protein-based building block of the present technology shows specific binding towards these plant proteins, that specific binding as described herein is lost when at least a cargo is attached to the at least one conjugation site comprised therein.

For example, if the precursor of the protein-based carrier building block specifically binds to fungi proteins (e.g., it is an anti-fungi protein ISVD, an anti-fungi protein DARPin, an anti-fungi protein affitin, an anti-fungi protein affibody, or the like), the protein-based carrier building block derived from it preferably does not specifically bind those fungi proteins (or binds those fungi proteins preferably with a K_{D} value greater than 5×10⁻⁴ mol/litre, as described herein). In other embodiments, the protein-based carrier building block specifically binds to fungi proteins, as its precursor does, but the specific binding is eliminated when at least a cargo is attached to the protein-based building block. Examples of fungi proteins to which the protein-based building block precursor (and/or protein-based building block of the present technology) may specifically bind are Cutinase, chitin, fungus sphingolipids. Hence, in one embodiment, the protein-based building block precursor, e.g., an ISVD, may specifically bind to at least one of these fungi proteins. The resulting protein-based building block may not specifically bind to the at least one of these fungi protein to which the precursor binds. If the protein-based building block of the present technology shows specific binding towards at least one of these fungi proteins, that specific binding as described herein is lost when at least a cargo is attached to the at least one conjugation site comprised therein.

For example, if the precursor of the protein-based carrier building block specifically binds to bacteria (e.g., it is an anti-bacterial ISVD, an anti-bacterial DARPin, an anti-bacterial affitin, an anti-bacterial affibody, or the like) and/or to bacterial molecules (e.g., it specifically binds one or more bacterial biomolecules, e.g., bacterial proteins, bacterial nucleic acids, bacterial lipids or bacterial glycans), the protein-based carrier building block derived from it preferably does not specifically bind those bacteria and/or bacterial molecules (or binds those bacteria and/or bacterial molecules preferably with a K_{D} value greater than 5×10⁻⁴ mol/litre, as described herein). In other embodiments, the protein-based carrier building block specifically binds to bacteria (e.g., it is an anti-bacterial ISVD, an anti-bacterial DARPin, an anti-bacterial affitin, an anti-bacterial affibody, or the like) and/or to bacterial molecules (e.g., it specifically binds one or more bacterial biomolecules, e.g., bacterial proteins, bacterial nucleic acids, bacterial lipids or bacterial glycans), as its precursor does, but the specific binding is eliminated when at least a cargo is attached to the protein-based building block. Examples of bacteria and bacterial molecules to which the protein-based building block precursor (and/or protein-based building block of the present technology) may specifically bind are the following: Beta-lactamase, tetanus toxin, Lactate Oxidase, Salmonella typhimurium, Helicobacter pylori, Mycobacterium tuberculosis, Clostridium difficile (toxin A and B), Pseudomonas aeruginosa, Bacillus anthracis, Botulinum Neurotoxin, Treponema pallidum, Chlamydia trachomatis, Escherichia coli, Campylobacter jejuni (flagella), Salmonella enterica, Bordetella pertussis (toxin), Shigella spp, Streptomyces venezuelae, chloramphenicol. Hence, in one embodiment, the protein-based building block precursor, e.g., an ISVD, may specifically bind to one or more of these bacteria (or their molecules, including biomolecules). The resulting protein-based building block may not specifically bind to the bacteria (or their molecules, including biomolecules) to which the precursor binds. If the protein-based building block of the present technology shows specific binding towards one or more of these bacteria (or molecules, including biomolecules, comprised therein), that specific binding as described herein is lost when at least a cargo is attached to the at least one conjugation site comprised therein.

For example, if the precursor of the protein-based carrier building block specifically binds to non-human animal proteins, such as snake proteins (e.g., it is an anti-snake protein ISVD, an anti-snake protein DARPin, an anti-snake protein affitin, an anti-snake protein affibody, or the like), the protein-based carrier building block derived from it preferably does not specifically bind those snake proteins (or binds those snake proteins preferably with a K_{D} value greater than 5×10⁻⁴ mol/litre, as described herein). In other embodiments, the protein-based carrier building block specifically binds to snake proteins, as its precursor does, but the specific binding is eliminated when at least a cargo is attached to the protein-based building block. Example of a snake protein to which the protein-based building block precursor (and/or protein-based building block of the present technology) may specifically bind is Cobra toxin. Hence, in one embodiment, the protein-based building block precursor, e.g., an ISVD, may specifically bind to this snake protein. The resulting protein-based building block may not specifically bind to the snake protein to which the precursor binds. If the protein-based building block of the present technology shows specific binding towards this snake protein, that specific binding as described herein is lost when at least a cargo is attached to the at least one conjugation site comprised therein.

For example, if the precursor of the protein-based carrier building block specifically binds to green fluorescent protein (GFP), which is a protein from Jellyfish (sea jellies) and corals, sea anemones, zoanithids, copepods and lancelets (e.g., it is an anti-GFP ISVD, an anti-GFP DARPin, an anti-GFP affitin, an anti-GFP affibody, or the like), the protein-based carrier building block derived from it preferably does not specifically bind GFP (or binds GFP preferably with a K_{D} value greater than 5×10⁻⁴ mol/litre, as described herein). In other embodiments, the protein-based carrier building block specifically binds to GFP, as its precursor does, but the specific binding is eliminated when at least a cargo is attached to the protein-based building block. Hence, in one embodiment, the protein-based building block precursor, e.g., an ISVD, may specifically bind to GFP. The resulting protein-based building block may not specifically bind to GFP to which the precursor binds. If the protein-based building block of the present technology shows specific binding towards GFP, that specific binding as described herein is lost when at least a cargo is attached to the at least one conjugation site comprised therein. For example, if the precursor of the protein-based carrier building block specifically binds to insect proteins (e.g., it is an anti-insect protein ISVD, an anti-insect protein DARPin, an anti-insect protein affitin, an anti-insect protein affibody, or the like), the protein-based carrier building block derived from it preferably does not specifically bind those insect proteins (or binds those insect proteins preferably with a K_{D} value greater than 5×10⁻⁴ mol/litre, as described herein). In other embodiments, the protein-based carrier building block specifically binds to insect proteins, as its precursor does, but the specific binding is eliminated when at least a cargo is attached to the protein-based building block. Examples of insect proteins to which the protein-based building block precursor (and/or protein-based building block of the present technology) may specifically bind are Androctonus autralis hecor toxins, chitin, chitin binding domain (CBD), V-ATPase subunit C, trehalase, cytochrome p450 monooxygenase, chitin deacetylase, chitin synthase and NPC1 sterol transporter. Hence, in one embodiment, the protein-based building block precursor, e.g., an ISVD, may specifically bind to at least one of these insect proteins. The resulting protein-based building block may not specifically bind to the at least one of these insect proteins to which the precursor binds. If the protein-based building block of the present technology shows specific binding towards at least one of these insect proteins, that specific binding as described herein is lost when at least a cargo is attached to the at least one conjugation site comprised therein.

For example, if the precursor of the protein-based carrier building block specifically binds to chitin, which is a crustaceans protein (e.g., it is an anti-chitin ISVD, an anti-chitin DARPin, an anti-chitin affitin, an anti-chitin affibody, or the like), the protein-based carrier building block derived from it preferably does not specifically bind chitin (or binds chitin preferably with a K_{D} value greater than 5×10⁻⁴ mol/litre, as described herein). In other embodiments, the protein-based carrier building block specifically binds to chitin, as its precursor does, but the specific binding is eliminated when at least a cargo is attached to the protein-based building block. Hence, in one embodiment, the protein-based building block precursor, e.g., an ISVD, may specifically bind to chitin. The resulting protein-based building block may not specifically bind to chitin to which the precursor binds. If the protein-based building block of the present technology shows specific binding towards chitin, that specific binding as described herein is lost when at least a cargo is attached to the at least one conjugation site comprised therein.

Hence, preferably, the protein-based carrier building block does not specifically bind to the precursor's target, should the protein-based carrier building block precursor have a target and should this be a non-human molecule (including biomolecules), such as a non-human protein. Hence, in one embodiment, the at least one protein-based building block comprised in the molecule of the present technology does not specifically bind any RSV protein, such as protein F of RSV, or binds any RSV protein, such as protein F of RSV, with a K_{D} (K_{D} value) greater than 5×10⁻⁴ mol/litre, as described herein. WO 2009/147248, Tables A-1 and A-2, provide examples of F-protein sequences. In one embodiment, the at least one protein-based building block comprised in the molecule of the present technology does not comprise/does not consist of an amino acid sequence selected from SEQ ID NO.: 1-34 as depicted on Tables A-1 and A-2 of WO 2016/055656. In another embodiment, the at least one protein-based building block comprised in the molecule of the present technology does not comprise/does not consist of the amino acid sequence as defined in SEQ ID NO.: 214.

In a further preferred embodiment, the protein-based building block of the present technology, when it has at least one cargo (such as a “model cargo”, e.g. a maleimide-modified alanine) attached to it (via the at least one conjugation sites or attachment points comprised therein) does not specifically bind to any molecule (including biomolecules) which the protein-based carrier building block precursor specifically binds to (i.e., the protein-based building block, with at least a cargo attached to it, preferably does not specifically bind to the precursor's target, e.g., a non-human protein or a non-protein molecule (including biomolecules)), or binds to any (non-human) molecule (including biomolecules) which the protein-based carrier building block precursor specifically binds to (i.e., the protein-based building block, with a cargo attached to it, preferably does also not specifically bind to the precursor's target, e.g., a non-human protein or a non-protein molecule) with a K_{D} value greater than 5×10⁻⁴ mol/litre, as described herein. Hence, in a preferred embodiment, if the protein based carrier building block of the present technology shows any specific binding towards a specific target, such as towards a molecule (including biomolecules, e.g., human, non-human animal, plant, microbial, viral, etc.), or towards a cell (e.g., animal, human, plant cell), microorganisms, virus, etc., that specific binding is eliminated when at least a cargo is attached to at least one attachment point or conjugation site comprised in the protein-based building block. In this specific embodiment, the cargo attached to the protein-based building block may of course show specific binding towards a target (including biomolecules, as described herein), but the protein-based building block does no longer specifically binds its target.

In a further preferred embodiment, the protein-based carrier building block of the present technology does not specifically bind to any human or non-human (e.g., non-human animal, plant, yeast, etc.) cell and/or cell type. If the building block shows any interaction with one or more human or non-human cells and/or cell types, such interaction is characterized by low specificity and/or low affinity, as defined herein. In particular, preferably, the carrier building block does also not specifically bind to any human or non-human cell and/or cell type to which the protein-based carrier building block precursor specifically binds (i.e., the protein-based building block preferably does also not specifically bind to the precursor's target, e.g., a non-protein molecule or a protein present on the surface of a human cell). The lack of binding to any human or non-human cell and/or cell type can for example be assessed with the “cell binding assay” as described below (see also, e.g., Hunter SA and Cochran JR, “Cell-binding assays for determining the affinity of protein-protein interactions: technologies and considerations”, Methods Enzymol., 2016, 580:21-44).

In another embodiment, the the protein-based carrier building block of the present technology does not specifically bind to any microorganism such as bacteria, fungi, protists, yeast and/or virus, or to any microbial or viral molecule (including biomolecules). If the building block shows any interaction with one or more microorganisms and/or virus, or with any microbial or viral molecule (including biomolecules), such interaction is characterized by low specificity and/or low affinity, as defined herein. In particular, preferably, the carrier building block does also not specifically bind to any microorganism and/or virus (or to any microbial or viral molecule (including biomolecules)) to which the protein-based carrier building block precursor specifically binds (i.e., the protein-based building block preferably does also not specifically bind to the precursor's target, e.g., a virus, a microorganism, a non-protein molecule (including biomolecules) or a protein present on the surface of a microorganism and/or virus). The lack of binding to any microorganism, or virus, or microbial molecule, or viral molecule can for example be assessed with the “cell binding assay” and/or SPR as described herein.

In another embodiment, the protein-based carrier building block of the present technology does not specifically bind to any microorganism such as bacteria, fungi, protists, yeast and/or virus (and/or to any microbial or viral molecule or biomolecule, such as microbial or viral proteins, nucleic acids, lipids, glycans, etc.) when it has at least one cargo (such as a “model cargo”, e.g. a maleimide-modified alanine) attached or conjugated to it (via the at least one attachment point or conjugation sites comprised therein). If the building block comprising the cargo attached to it shows any interaction with one or more microorganisms and/or virus (or with any microbial or viral molecule or biomolecule, such as microbial or viral proteins, nucleic acids, lipids, glycans, etc.), such interaction is characterized by low specificity and/or low affinity, as defined herein. In particular, preferably, the carrier building block does also not specifically bind to any microorganism and/or virus (or to any microbial or viral molecule or biomolecule, such as microbial or viral proteins, nucleic acids, lipids, glycans, etc.) to which the protein-based carrier building block precursor specifically binds when the protein-based carrier building block has at least one cargo attached or conjugated to it (i.e., the protein-based building block preferably does also not specifically bind to the precursor's target, e.g., a non-protein molecule or biomolecule or a protein present on the surface of a microorganism and/or virus, or present in the microorganism or virus, when it has at least one cargo attached or conjugated to it). For instance, the protein-based building block of the present technology does not specifically bind to any viruses and/or viral proteins, such as RSV and/or one or more proteins of RSV, such as protein F of RSV, when the building block has at least a cargo attached or conjugated to it. Hence, for instance, the protein-based carrier building block (e.g., a DARPin-based carrier building block) may show specific binding towards a microorganism and/or a virus, such as RSV and/or RSV proteins, such as protein F of RSV, but the specific binding (as defined herein), if any, is lost when at least one cargo is attached or conjugated to the protein-based building block.

The lack of specific binding to any microorganism can for example be assessed with the “cell binding assay” as described herein. The lack of specific binding to viruses, microbial and/or viral molecules or biomolecules can, for example, be assessed by surface plasmon resonance, as described herein.

In another embodiment, the protein-based carrier building block of the present technology does not specifically bind to any molecule, including biomolecules, including human molecules and non-human molecules (including human and non-human biomolecules, e.g., human and/or non-human proteins, human and/or non-human nucleic acids such as DNA and/or RNA, human and/or non-human lipids (e.g., such as phosphatidylserine (PS)) or non-human glycans), or binds to any molecule, including bio molecules, including human molecules and non-human molecules (including human and non-human biomolecules, e.g., human and/or non-human proteins, nucleic acids such as DNA and/or RNA, lipids (e.g., such as phosphatidylserine (PS)) or glycans) with a K_{D} (K_{D} value) greater than 5×10⁻⁴ mol/litre, as described herein. For instance, the protein-based carrier building block does not specifically bind to any human and/or non-human animal biomolecule (e.g., human and/or non-human animal proteins, human and/or non-human nucleic acids such as DNA and/or RNA, human and/or non-human lipids (e.g., such as phosphatidylserine (PS)) or human and/or non-human glycans), or binds to any human and/or non-human animal biomolecule with a K_{D} (K_{D} value) greater than 5×10⁻⁴ mol/litre, as described herein. For instance, the protein-based carrier building block does not specifically bind to any bacterial molecule (including bacterial biomolecules, e.g., bacterial proteins, nucleic acids such as DNA and/or RNA, lipids (e.g., such as phosphatidylserine (PS)) or glycans), or binds to any bacterial molecule, as defined above, with a K_{D} (K_{D} value) greater than 5×10⁻⁴ mol/litre, as described herein. For instance, the protein-based carrier building block of the present technology does not specifically bind to any viral molecule (including biomolecules, e.g., viral proteins, nucleic acids such as DNA and/or RNA, lipids (e.g., such as phosphatidylserine (PS)) or glycans), or binds to any viral molecule, as defined herein, with a K_{D} (K_{D} value) greater than 5×10⁻⁴ mol/litre, as described herein. For instance, the protein-based carrier building block does not specifically bind to any fungi molecule (including biomolecules, e.g., fungi proteins, nucleic acids such as DNA and/or RNA, lipids (e.g., such as phosphatidylserine (PS)) or glycans), or binds to any fungi molecule, as defined herein, with a K_{D} (K_{D} value) greater than 5×10⁻⁴ mol/litre, as described herein. For instance, the protein-based carrier building block does not specifically bind to any yeast molecule (including biomolecules, e.g., yeast proteins, nucleic acids such as DNA and/or RNA, lipids (e.g., such as phosphatidylserine (PS)) or glycans), or binds to any yeast molecule, as described herein, with a K_{D} (K_{D} value) greater than 5×10⁻⁴ mol/litre, as described herein. For instance, the protein-based carrier building block does not specifically bind to any plant molecule (including biomolecules, e.g., plant proteins, nucleic acids such as DNA and/or RNA, lipids (e.g., such as phosphatidylserine (PS)) or glycans), or binds to any plant molecule, as defined herein, with a K_{D} (K_{D} value) greater than 5×10⁻⁴ mol/litre, as described herein. For instance, the protein-based carrier building block does not specifically bind to any mammalian molecule (including mammalian biomolecules, e.g., mammalian proteins, nucleic acids such as DNA and/or RNA, lipids (e.g., such as phosphatidylserine (PS)) or glycans), or binds to any mammalian molecule, as defined herein, with a K_{D} (K_{D} value) greater than 5×10⁻⁴ mol/litre, as described herein.

In the context of the present technology, the term “biomolecule” or “biological molecule” refers to molecules present in organisms, including animals, plants, microorganisms that play a role in one or more biological processes, such as cell division, morphogenesis, or development. Biomolecules are the building blocks of life and perform important functions in living organisms. Biomolecules include the primary metabolites which are large macromolecules such as proteins, carbohydrates (glycans), lipids (e.g., such as PS), and nucleic acids (such as DNA, RNA), as well as small molecules such as vitamins and hormones. The four major types of biomolecules are carbohydrates (glycans), lipids, nucleic acids, and proteins.

In a further preferred embodiment, the protein-based carrier building block of the present technology does not specifically bind any non-human protein and/or any non-protein molecule (including biomolecule) when at least one cargo (such as a “model cargo”, e.g. a maleimide-modified alanine) is conjugated to the at least one attachment point or conjugation site on the protein-based carrier building block, preferably it does not specifically bind any non-human protein and/or any non-protein molecule (including biomolecule) to which the protein-based carrier building block precursor specifically binds, or binds to them with a K_{D} (K_{D} value) greater than 5×10⁻⁴ mol/litre, as described herein.

Hence, in one embodiment, the present technology provides a molecule comprising at least one protein-based carrier building block as described in the present technology, wherein the protein-based carrier building block has at least a cargo (such as a “model cargo”, e.g. a maleimide-modified alanine) attached or conjugated to it (via the at least one attachment point or conjugation site comprised in the protein-based carrier building block), and wherein the protein-based carrier building block does not specifically bind to any molecule (including biomolecules) and/or organisms (such as cells, microorganisms, virus, etc.). Hence, in one embodiment, the protein-based building block, when at least a cargo is conjugated to it, loses its target binding specificity. For instance, the protein-based building block, comprising a cargo attached to it, does not specifically bind to any molecule (including biomolecules) and/or organisms (such as cells, microorganisms, virus, etc.) which the protein-based carrier building block precursor specifically binds (i.e., the protein-based building block, with at least a cargo attached to it, preferably does not specifically bind to the precursor's target, or binds to any (non-human) molecule (including biomolecules) and/or organisms (such as cells, microorganisms, virus, etc.) which the protein-based carrier building block precursor specifically binds to (i.e., the protein-based building block, with a cargo attached to it, preferably does also not specifically bind to the precursor's target) with a K_{D} value greater than 5×10⁻⁴ mol/litre, as described herein.

The skilled person is aware of means for reducing and/or eliminating specific binding of a protein-based carrier building block precursor to proteins and/or non-protein molecules (including biomolecules). For instance, mutations may be performed in the amino acid sequence of the precursor building block so that it no longer specifically binds to human proteins, or to any non-human protein, or to non-protein molecules (including biomolecules), or binds to them with a K_{D} (K_{D} value) greater than 5×10⁻⁴ mol/litre, as described herein.

The affinity of a molecular interaction between two molecules (e.g., of two biomolecules) can be measured via different techniques known per se, such as the well-known surface plasmon resonance (SPR) biosensor technique (see for example Ober et al. 2001, Intern. Immunology 13:1551-1559, in particular section “Surface plasmon resonance (SPR) experiments” starting on p. 1552, which describes conditions for measuring the affinity of a molecular interaction between two molecules, or the explanations provided herein in this description). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a Cytiva lifesciences company, Uppsala, Sweden and Piscataway, NJ). For further descriptions, see Jonsson et al. (1993, Ann. Biol. Clin. 51:19-26), Jonsson et al. (1991 Biotechniques 11:620-627), Johnsson et al. (1995, J. Mol. Recognit. 8:125-131), and Johnnson et al. (1991, Anal. Biochem. 198:268-277). For instance, the affinity (K_{D}) of a molecular interaction between two molecules can be determined via SPR on a ProteOn XPR36 instrument (Bio-Rad Laboratories). The experiment can be performed at 25° C., and as assay buffer PBS pH7.4 containing 0.005% Tween 20 (Bio-Rad Laboratories) can be used. Targets such as human proteins or non-protein molecules (biomolecules), or non-human biomolecules, as described herein, such as nucleic acids (e.g., DNA, RNA), lipids (e.g., such as phosphatidylserine (PS)) or glycans can be immobilized onto different ligand lanes from a GLC sensorchip (Bio-Rad Laboratories), e.g., with the ProteOn Amine Coupling Kit (Bio-Rad Laboratories) according to the manufacturer's instructions. The protein-based building blocks of the present technology can be captured on the target immobilized ligand lanes. One ligand lane can serve as a reference surface and no protein-based building block is captured on the surface. Different concentrations (e.g., ranging from 300 nM to 1.2 nM) diluted in running buffer can be flowed over the respective protein-based building blocks and reference surface in multi-cycle kinetics for 2 minutes, followed by a constant flow of the assay buffer for 15 minutes. Between the different injections, the surfaces can be regenerated with 3 M MgCl₂ (Cytiva), or with 10 mM Glycine pH 1.5 (Cytiva). Several buffer blanks can be injected for double referencing. Data can be analyzed, e.g., with the ProteOn Manager 3.1.0 software (Bio-Rad Laboratories). The kinetic rate constants (ka and kd) can be calculated by fitting the sensorgrams via the Langmuir 1:1 interaction ligand binding model. The equilibrium dissociation constant K_{D} can be calculated as the kd/ka ratio. See also, e.g., https://nicoyalife.com/wp-content/uploads/2023/02/characterization-of-Influenza-using-Alto.pdf.

Another well-known biosensor technique to determine affinities of biomolecular interactions is bio-layer interferometry (BLI) (see for example Abdiche et al. 2008, Anal. Biochem. 377:209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al., “Characterizing high-affinity antigen/antibody complexes by kinetic- and equilibrium-based methods”, Anal. Biochem., 2004, 328:35-43), using the KinExA® platform (Sapidyne Instruments Inc, Boise, USA). The “term “KinExA”, as used herein, refers to a solution-based method to measure true equilibrium binding affinity and kinetics of unmodified molecules. Equilibrated solutions of a binding unit/target complex, such as an antibody/antigen complex, are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

Further, the GYROLAB® immunoassay system provides a platform for automated bioanalysis and rapid sample turnaround (Fraley et al., “The Gyrolab™ immunoassay system: a platform for automated bioanalysis and rapid sample turnaround”, Bioanalysis 2013, 5:1765-74). Further, the affinity of a molecular interaction between two molecules (e.g., between two biomolecules such as between two proteins), or between one biomolecule such as one protein and one cell, can be measured using flow cytometry to analyze ligand binding to antigens such as proteins, lipids (e.g., such as phosphatidylserine (PS)), sugars, etc. presented on the surface of a cell (“cell binding assay”). The skilled person is familiar with cell-binding assays to determine the affinity of a certain soluble molecule (such as the molecule of the present technology) and a binding partner present on the surface of a cell, such as a human cell. For instance, Hunter S. A. and Cochran J. R. (“Cell-binding assays for determining the affinity of protein-protein interactions: technologies and considerations”, Methods Enzymol. 2016, 580: 21-44), present a practical guide for measuring binding events between soluble ligands and binding partners expressed on the surface of, inter alia, mammalian cells. For instance, as shown in the examples, the cell binding assay can be carried out as follows:
a. Adding a fixed number of (human or non-human) cells to a 96-well V-bottom plate (e.g., 50 μL human cell suspension (e.g., 5E+04/96-well), or to tubes, such as Eppendorf tubes, in cold fluorescence-activated cell sorting (FACS) buffer (e.g., consisting of D-PBS, 2% heat inactivated fetal bovine serum (HI FBS) and 0.05% Sodium Azide);
b. Optionally performing a washing step;
c. Adding the soluble molecule, e.g., the molecule of the present technology, or the protein-based building block of the present technology, preferably marked, such as with a fluorescent label or an epitope tag, and incubate for a certain amount of time, until the reaction has come to equilibrium, generally a number of hours, e.g., for about 3h, at low temperature, typically 4° C. while shaking;
d. Evaluating the binding of the soluble molecule to the human cells by flow cytometry, e.g., by FACS.

Hence, the cell-binding assay can be performed by adding a number of cells (human or non-human, such as non-human animal, plant, microorganisms, etc.) to a recipient (e.g., 96-well V-bottom plate or tubes, as described above), preferably in a physiological buffer, adding the molecule which binding is to be assessed (e.g., the molecule of the present technology, or the protein-based building block of the present technology), which is preferably marked, and incubate it with the cells for an amount of time, e.g., when the reaction has come to equilibrium, generally a number of hours, e.g., for about 3 h, preferably at low temperature, typically 4° C., preferably while shaking, and finally evaluating the binding of the soluble molecule to the human cells by flow cytometry, e.g., by FACS.

In order to calculate the binding affinities (e.g., K_{D} and/or EC₅₀) between a soluble molecule, e.g., the molecule of the present technology, and a human cell, the soluble molecule in step c. above may be added to each well/tube at varying concentrations, spanning two orders of magnitude above and below the anticipated K_{D} and/or EC₅₀. Binding values can be determined from the average signal value (e.g., average fluorescence value) of each sample, plotting the fraction bound vs. ligand concentration (log scale) and fitting a sigmoidal curve using nonlinear regression analysis. The ligand concentration at half the fraction bound, also referred to as EC₅₀, will be a first approximation of the equilibrium dissociation constant (K_{D}).

The skilled person is able to determine whether a molecule is able to specifically bind human proteins, as defined in the context of the present technology. For instance, the skilled person may make use of commercially available protein arrays to determine the binding affinity of a certain molecule (protein) towards human proteins. For instance, the skilled person may make use of the commercially available Proteome Profiler™ Antibody Arrays, which allows for the semiquantitative measurement of more than 100 proteins in a single sample. Alternatively or additionally, the skilled person may make use of, for example, HuProt™ assay, such as the version v4.0, which consists of >21,000 unique human proteins, isoform variants, and protein fragments—covering 16,794 unique genes. This includes 15,889 of the 19,613 canonical human proteins described in the Human Protein Atlas, with broad coverage across protein subclasses. The skilled person can also use commercially available cell arrays, such as human, non-human animal, plant, bacteria, yeast, etc. arrays to determine the binding affinity (e.g., K_{D} and/or EC₅₀) of a certain molecule (protein) towards human cells. See also, e.g., Example 16.

Similarly, the skilled person is able to determine whether a molecule is able to specifically bind a non-human protein, such as a bacterial or viral protein. For instance, the skilled person may make use of protein-binding assays to determine the binding affinity of a certain molecule (e.g., a protein) towards non-human (such as bacterial or viral) proteins. Similarly, the skilled person is able to determine whether a molecule (e.g., a protein) is able to specifically bind a non-protein molecule, e.g., a human non-protein molecule, such as human DNA, human RNA, human lipids (e.g., such as phosphatidylserine (PS)) or human glycans, see, e.g., Campanero-Rhodes M A et al., “Microarray strategies for exploring bacterial surface glycans and their interactions with glycan-binding proteins”, Front Microbiol. 2020, 10:2909. For instance, as described above, the binding affinity of a molecular interaction between two molecules (such as two proteins, or a protein and a non-protein molecule) can be measured by SPR. SPR allows for the determination of the K_{D} of a potential interaction between two molecules, as described in detail above.

As it will be evident to the skilled person, the at least one carrier building block comprised in the molecule of the present technology may show non-specific binding with one or more human proteins (and/or with one or more non-human proteins, and/or with one or more non-protein molecules, such as human non-protein molecules, and/or with one or more human cell types as described above). This is because there may be molecular forces between the at least one carrier building block of the present technology and one or more human proteins (and/or one or more non-human proteins, and/or one or more non-protein molecules, such as human non-protein molecules, and/or one or more human cells, as described above), e.g., in the form of hydrophobic interactions, hydrogen bonding, Van der Waals interactions, and other nonspecific interactions. Hence, if this happens, the at least one carrier building block comprised in the molecule of the present technology may non-specifically bind to one or more human proteins (and/or to one or more non-human proteins, and/or to one or more non-protein molecules, such as human non-protein molecules, and/or to one or more human cells, if this is the case, as described above). In the context of the present technology, any K_{D} value greater than 5×10⁻⁴ mol/litre (or any K_{A} value lower than 2×10³ litres/mol) is generally considered to represent “non-specific binding”. Hence, the building block may bind to any human protein (or non-human protein, and/or to any human cell, if this is the case, as explained above) with a K_{D} (K_{D} value) greater than 5×10⁻⁴ mol/litre (or with a K_{A} value lower than 2×10³ litres/mol), such as with a K_{D} (K_{D} value) greater than 5.5×10⁻⁴ mol/litre (or with a K_{A} value lower than 1.8×10³ litres/mol), or with a K_{D} (K_{D} value) greater than 6×10⁻⁴ mol/litre (or with a K_{A} value lower than 1.7×10³ litres/mol). In addition, in the context of the present technology, in a preferred embodiment, the carrier building block may bind to any non-protein molecule, such as to any human non-protein molecule (e.g., DNA, RNA, lipids (e.g., such as phosphatidylserine (PS)), glycans) with a K_{D} (K_{D} value) greater than 5×10⁻⁴ mol/litre (or with a K_{A} value lower than 2×10³ litres/mol), such as with a K_{D} (K_{D} value) greater than 5.5×10⁻⁴ mol/litre (or with a K_{A} value lower than 1.8×10³ litres/mol), or with a K_{D} (K_{D} value) greater than 6×10⁻⁴ mol/litre (or with a K_{A} value lower than 1.7×10³ litres/mol). In addition, in the context of the present technology, the building block may bind to any human cell with a K_{D} (K_{D} value) greater than 5×10⁻⁴ mol/litre (or with a K_{A} value lower than 2×10³ litres/mol), such as with a K_{D} (K_{D} value) greater than 5.5×10⁻⁴ mol/litre (or with a K_{A} value lower than 1.8×10³ litres/mol), or with a K_{D} (K_{D} value) greater than 6×10⁻⁴ mol/litre (or with a K_{A} value lower than 1.7×10³ litres/mol). In the context of the present technology, this binding affinity is considered to be “non-specific binding”.

In one embodiment, the protein-based carrier building block of the present technology is not derived from the crystallizable fragment of an antibody (Fc, which contains two C_{H}2 and two C_{H}3 domains) such as the Fc fragment of a monoclonal antibody (mAb). In another embodiment, the protein-based carrier building block of the present technology is not derived from the C_{H}2 and/or the C_{H}3 domains of the Fc fragment. In another embodiment, the protein-based carrier building block of the present technology is not derived from a C_{H}1 and/or the C_{L} domains comprised in the antigen-binding fragment (Fab) of an antibody, such as the C_{H}1 and/or the C_{L} domains comprised in the Fab of a mAb. In one embodiment, the molecule of the present technology is not (or is not derived from) a crystallizable fragment (Fc) of an antibody, such as a mAb. In another embodiment, the molecule of the present technology is not (or is not derived from) the Fab of an antibody, such as a mAb.

In one embodiment, the molecule of the present technology does not comprise V_{H}-V_{L} pairs or, e.g., it does not comprise at least one V_{H} and at least one V_{L} which interact (are bound to) with each other, such as in an antibody. In another embodiment, the molecule of the present technology does not comprise C_{L}-C_{H}1 conjugates, e.g., it does not comprise at least one C_{L} and at least one C_{H}1 which are linked to each other, e.g., through a disulphide bridge.

### Attachment Points or Conjugation Sites

As mentioned above, the carrier building block present in the molecule of the present technology has at least one attachment point (also referred to as conjugation site in the present disclosure), preferably at a solvent-accessible position, as defined further below. Preferably, the at least one protein-based carrier building block comprises more than one attachment points or conjugation sites, preferably at solvent-accessible positions. In a preferred embodiment, the protein-based carrier building block comprises at least two attachment points or conjugation sites. In another embodiment, the protein-based building block comprises three conjugation sites or more, such as six or nine conjugation sites. For instance, the protein-based carrier building block may have two, three, four, five, six, seven, eight, nine, ten conjugation sites or more. In one embodiment, if more than one, the conjugation sites present in the carrier building block are different from each other. For instance, if the carrier building block comprises two conjugation sites, these conjugation sites may be functionally/chemically different from each other, i.e., each conjugation site or attachment point is chemically different from each other (e.g., if there are two conjugation sites, one conjugation site may be a —SH group present in the side chain of a cysteine located in a solvent-accessible position, and the other conjugation site may be a —NH₂ group present in the side chain of a lysine located in a solvent-accessible position). If the building block has more than two conjugation sites (e.g., at least three conjugation sites, such as three, four, five, six, seven, eight, nine, ten, etc.), there may be at least two types of conjugation sites among the at least three conjugation sites present in the building block. In another embodiment, if the building block has three conjugation sites, each conjugation site is functionally different from each other. In another embodiment, if the building block has three conjugation sites, two conjugation sites are the same and one conjugation site is functionally different from the other two conjugation sites. In another embodiment, all conjugation sites present in the building block are functionally different from each other. In another embodiment, all conjugation sites present in the carrier building block are the same. For instance, the protein-based building block may comprise one, two, three, four, five, six, seven, eight, nine, ten or more conjugation sites which are all the same, e.g., which are all —SH groups present in the side chain of cysteines located at solvent-accessible positions in the protein-based building block.

In another embodiment, alternatively or additionally, if there is more than one conjugation site, the conjugation sites are spatially distant from each other (spatially separated from each other). The skilled person will appreciate that the minimal distance between conjugation sites will be dictated by the nature of the cargos (and linkers, if used) which are to be attached or conjugated to the attachment points or conjugation sites in the protein-based carrier building block. For larger cargos (e.g., ISVDs), the minimal distance can still be kept small when used in combination with long linkers, which add the needed flexibility and the envisaged target binding. A short distance between conjugation sites, combined with short linkers, if any, will likely limit the target binding of larger cargos, and result in restricted engagement (e.g., increased cell specificity). In addition, the solubility of the molecule may be decreased (i.e., the molecule may be more prone to aggregation). On the other hand, if the cargos to be attached are rather small (e.g., radioactive isotopes), the minimal distance can be kept small even in the absence of linkers, see, e.g., Example 5 below. Hence, the skilled person will be able to select the location of the specific conjugation sites and the length and flexibility of the linkers, if any, depending on the nature of the cargos which are to be attached or conjugated to the protein-based carrier building block.

A “conjugation site” or “attachment point” may be a reactive group in the side chain of a natural or a non-natural (also referred to as “noncanonical”, “unnatural” or “unusual”, as described above) amino acid preferably located at a solvent-accessible position in the protein-based carrier building block. It may also be the C-terminal and/or N-terminal reactive group (—COOH and —NH₂ groups, respectively) of the protein-based carrier building block. In the context of the present technology, a “reactive group in the side chain of an amino acid” (either natural or non-natural, as defined above) refers to any chemical group present in the side chain of an amino acid which is capable of forming a covalent bond. For instance, if the amino acid is lysine (or ornithine (Orn), or Diaminopropionic acid (Dap), or Diaminobutyric acid (Dab)), the reactive group present on its side chain is a primary amine. For example, if the amino acid is cysteine, the reactive group present on its side chain is a thiol group. For example, if the amino acid is aspartic or glutamic acid, the reactive group present in their side chain is a carboxylic group. For example, if the amino acid is tyrosine, the reactive group present on its side chain is a phenolic hydroxyl group. For example, if the amino acid is arginine, the reactive group present on its side chain is a guanidino group. For example, if the amino acid is methionine, the reactive group present on its side chain is a thioether group.

In the context of the present technology, the “C-terminal or N-terminal reactive group of the protein-based carrier building block” refers to the —COOH and —NH₂ reactive groups present in the C- and N-terminal amino acid of the protein-based carrier building block. If the carrier building block does not have a free C- and/or N-terminal end (e.g., because the carrier building block is C- and/or N-terminal linked to another protein-based building block, or to another peptide or protein, or because the N-terminal is amidated, or because the C-terminal is acetylated, etc.), then the N- and C-terminal ends of the carrier building block are not suitable as attachment points or conjugation sites as defined herein. In some embodiments the “conjugation site” or “attachment point” is not the C-terminal or N-terminal reactive group of the protein-based carrier building block.

The at least one conjugation site or attachment point present in the building block of the present technology may be already present in the building block precursor (e.g., the —NH₂ group in the side chain of a lysine present in the building block precursor, preferably at a solvent-accessible position) or may be engineered. Preferably, at least one or more of the attachment points or conjugation sites of the protein-based building block are engineered. In the context of the present technology, an “engineered” attachment point or conjugation site means a conjugation site or attachment point which is present in the protein-based carrier building block but which was not present in its precursor at the same or corresponding position. For instance, the protein-based building block precursor may be modified to introduce one or more attachment points or conjugation sites, as described in detail below. A non-limiting example of an engineered attachment point or conjugation site is a reactive group present in the side chain of an amino acid in the protein-based carrier building block which amino acid was not present at the same or equivalent position in the building block precursor. For instance, if the building block precursor has a serine at a certain position X (which is preferably a solvent-accessible position) in the building block precursor, and that serine is mutated to a cysteine in the carrier building block, the —SH group of that cysteine would be an engineered attachment point or conjugation site. For instance, if an amino acid (e.g., a Cys, or a Tyr) is added at the N- or C-terminal end of the building block precursor, the reactive group present in the side chain of that newly added amino acid in the carrier building block would be an engineered attachment point or conjugation site.

Hence, in a preferred embodiment, the protein-based carrier building block of the present technology has at least two conjugation sites or attachment points, wherein at least one, preferably at least two of them are engineered attachment points or conjugation sites, i.e., they were not present in the building block precursor at the same or corresponding position. In another preferred embodiment, all of the conjugation sites or attachment points present in the protein-based building block are engineered attachment points or conjugation sites, i.e., they were not present in the building block precursor at the same or corresponding position. In one embodiment, the carrier building block has two or more engineered attachment points or conjugation sites, such as three, four, five, six, seven, eight, nine, ten or more engineered attachment points or conjugation sites.

As used herein a residue position in one polypeptide sequence “corresponds to” a residue position in another polypeptide sequence if it exists in an equivalent position in the polypeptide sequence, as indicated, e.g., by primary sequence homology or functional equivalence or Kabat numbering. A corresponding position may be identified by alignment of the two polypeptide sequences. The alignment used to identify a corresponding position or corresponding region may be obtained using a conventional alignment algorithm such as Blast (Altschul et al., “Basic local alignment search tool”, J Mol Biol., 1990, 215 (3): 403-10).

The at least one conjugation site present in the carrier building block of the present technology may be free (i.e., ready for reaction) or capped/protected. Hence, the α-amino group, the carboxylic acid terminus, or the reactive groups present in the side chain of one or more amino acids of the carrier building block (e.g., amines, carboxylic acids, alcohols, thiols) may be capped or protected with a protecting group, e.g., to prevent polymerization of the amino acids, to minimize undesirable side reactions during the synthesis of the building block or to selectively attach different cargos, for example. Of course, if the at least one conjugation site is capped or protected, it has to be de-capped or deprotected before attaching or conjugating a cargo to it, as described in detail below.

Thus, the at least one conjugation site present in the protein-based carrier building block of the present technology may be (non-limiting) a primary amine, a thiol group, a hydroxyl group, a guanidino group, a carboxyl group or a thioether group. For instance, the conjugation site may be a free or capped (protected) thiol group.

Hence, in some embodiments, the at least one conjugation site present in the protein-based carrier building block of the present technology may be a primary amine present in the side chain of a lysine (or ornithine (Orn), or Diaminopropionic acid (Dap), or Diaminobutyric acid (Dab)) in the protein-based building block, preferably located at a solvent-accessible position. In other embodiments, the conjugation site is a thiol group present in the side chain of a cysteine in the protein-based building block, preferably located at a solvent-accessible position in the protein-based building block. In other embodiments, the conjugation site is a carboxylic group present in the side chain of an aspartic or glutamic acid in the protein-based building block, preferably located at a solvent-accessible position in the protein-based building block. In other embodiments, the conjugation site is a guanidino group present the side chain of an arginine in the protein-based building block, preferably located at a solvent-accessible position in the protein-based building block. In other embodiments, the conjugation site is a thioether group present the side chain of a methionine in the protein-based building block, preferably located at a solvent-accessible position in the protein-based building block. In other embodiments, the conjugation site is the phenolic OH-group of a tyrosine in the protein-based building block, preferably located at a solvent-accessible position in the protein-based building block. In one embodiment, the tyrosine is preferably located at the N- or C-terminal end of the protein-based carrier building block of the molecule. In other embodiments, the conjugation site is the N-terminal primary amine of the carrier building block, if this is free and preferably solvent-accessible. In other embodiments, the conjugation site is the C-terminal carboxyl group of the carrier building block, if this is free and preferably solvent-accessible.

As described above, the conjugation sites may be free or protected. For instance, as already described above, if the conjugation site is a thiol group (e.g., from a cysteine in the protein-based building block, preferably located at a solvent-accessible position in the protein-based building block), the thiol group may be free (—SH) or protected/capped. A capped thiol group refers to a thiol group which is (reversibly) protected with a protecting group (e.g., with another cysteine, with glutathione (GSH), with cysteamine or with protecting groups such as Benzyl (Bzl, Bn), Trityl (Trt), Diphenylmethyl (Dpm, Bzh, Bh), Tetrahydropyranyl (Thp), tert-Butyl (tBu), etc.). Spears, R., et al., (“Cysteine protecting groups: applications in peptide and protein science”, Chem. Soc. Rev., 2021, 50, 11098) provides a review on the different cysteine protecting groups. In addition, Isidro-Llobet, A., et al., (“Amino acid-protecting groups”, Chem Rev., 2009, 109 (6): 2455-504) provides a review of different amino acid protecting groups.

In one embodiment, the protein-based carrier building block of the present technology comprises at least two attachment points or conjugation sites which are two reactive groups present in the side chain of two amino acids (which may be natural or a non-natural) in the protein-based building block, preferably located at solvent-accessible positions in the protein-based carrier building block. For instance, in one embodiment, the protein-based carrier building block comprises at least two attachment points or conjugation sites which are two reactive groups present in the side chain of two natural amino acids (e.g., two Cys) in the protein-based building block, preferably located at solvent-accessible positions in the protein-based carrier building block.

In one embodiment, at least one of the attachment points or conjugation sites present in the protein-based building block is linked (directly or via a linker) to a cargo, as defined herein. In a preferred embodiment, the molecule of the present technology comprises at least one protein-based carrier building block and at least one cargo, wherein the at least one cargo is attached or conjugated to the at least one protein-based carrier building block through the at least one attachment point or conjugation site. A “cargo” may be any molecule which is/may be attached or conjugated to the protein-based carrier building block through the attachment point(s) or conjugation site(s) present therein. For instance, cargos which may be attached or conjugated to the protein-based carrier building block of the present technology are proteins, peptides, ISVDs (such as V_{HH}, V_{L} or V_{H}), polyethylene glycol (PEG), small molecules (such as, e.g., cryptophycin, DM4), glycans (such as, e.g., M6P), lipids, chelators, fluorophores, radio isotopes, vitamins (such as folic acid or biotin), nucleic acids such as oligonucleotides or siRNA, etc. The cargo may have different functionalities. For instance, the at least one cargo may be a half-life extending (HLE) molecule, a targeting molecule, a therapeutic molecule or precursor thereof, an imaging molecule, a toxic molecule, an agonist (such as a Toll-like receptor (TLR) agonist), a T-cell engagement molecule, a sweeping/degrader molecule, a cell-penetrating molecule, a nuclear localization molecule, a blood brain barrier (BBB) shuttle, a radiotherapeutic molecule or an imaging probe.

Hence, in another embodiment, the molecule of the present technology comprises at least one protein-based carrier building block and at least one cargo, wherein the cargo is attached or conjugated to the at least one protein-based carrier building block through the at least one attachment point or conjugation site, and wherein the cargo is a HLE molecule, such as an albumin-binding ISVD (as described herein, e.g., as defined in Table 8, such as SEQ ID NO.: 63 or 106) or a PEG molecule, or ELNN polypeptides, as described herein. In another embodiment, the molecule of the present technology comprises at least one protein-based carrier building block and at least one cargo, wherein the cargo is attached or conjugated to the at least one protein-based carrier building block through the at least one attachment point or conjugation site, and wherein the cargo is a targeting moiety and/or a therapeutic moiety as described herein. In a further embodiment, the molecule of the present technology comprises at least one protein-based carrier building block and at least two cargos, wherein the cargos are attached or conjugated to the at least one protein-based carrier building block through at least two attachment points or conjugation sites, wherein the at least two cargos are one HLE molecule, as described herein, and one therapeutic and/or targeting moiety, as described herein.

In one embodiment, the at least one protein-based carrier building block comprised in the molecule of the present technology comprises at least two cysteines, preferably located at solvent accessible positions, such as three cysteines, or six cysteines, or nine cysteines, preferably located at solvent accessible positions, with free or capped thiol groups that are the at least two, such as three, or six, or nine, conjugation sites as defined herein. In one embodiment, the at least one protein-based carrier building block comprised in the molecule of the present technology comprises three cysteines, preferably located at solvent accessible positions, with free or capped thiol groups that are the three conjugation sites as defined herein. In one embodiment, the protein-based carrier building block does not comprise any other cysteine at solvent accessible positions besides the three cysteines at solvent-accessible positions which bear the three conjugation sites (free or capped thiol groups) (but may comprise one or more cysteines at positions which are not solvent-accessible). In another embodiment, the at least one protein-based carrier building block comprised in the molecule of the present technology comprises four, five, six, seven, eight, nine, ten or more cysteines, preferably located at solvent accessible positions, with free or capped thiol groups that are the four, five, six, seven, eight, nine, ten or more conjugation sites as defined herein. In one embodiment, the protein-based carrier building block does not comprise any other cysteine at solvent accessible positions besides the four, five, six, seven, eight, nine, ten or more cysteines which bear the four, five, six, seven, eight, nine, ten or more conjugation sites (free or capped thiol groups) and which are located at solvent-accessible positions in the building block. In other embodiments, the at least one protein-based building block comprised in the molecule of the present technology comprises at least one amino acid, such as one, two, three, four, five, six, seven, eight, nine, ten or more, which may be natural or non-natural, preferably located at solvent accessible positions, which comprises a reactive group on its side chain which is the conjugation site as defined herein. In another embodiment, the at least one protein-based building block comprised in the molecule of the present technology comprises at least two conjugation sites, one of which is a (free or protected) thiol group from a cysteine preferably located at a solvent-accessible position in the protein-based carrier building block, and the other one is a-OH group from a tyrosine preferably located at a solvent-accessible position in the protein-based carrier building block, preferably from a N- or C-terminally exposed tyrosine. In another embodiment, the at least one protein-based building block comprised in the molecule of the present technology comprises at least two conjugation sites, one of which is a (free or protected) thiol group from a cysteine preferably located at a solvent-accessible position in the protein-based carrier building block, and the other one is a reactive group from a non-natural amino acid preferably located at a solvent-accessible position in the protein-based carrier building block.

In one embodiment, a conjugation site or attachment point in the protein-based building block is a selenol (—HSe) group from a selenocysteine (Sec or U), which may be located, e.g., in the C-terminal of the protein-based carrier building block. In another embodiment, a conjugation site or attachment point in the protein-based building block is a keto group of a p-acetylphenylalanine (pAcPhe), that can be selectively coupled to an alkoxyamine derivatized cargo, see, e.g., Jun Y. Axup et al., “Synthesis of site-specific antibody-drug conjugates using unnatural amino acids”, PNAS, 2012, 109 (40) 16101-16106.

The skilled person is aware of ways of incorporating one or more unnatural amino acids in the at least one protein-based building block comprised in the molecule of the present technology, if this is the case. For instance, WO 2021/050554, the content of which is herewith incorporated by reference, describes in detail how to incorporate one or more unnatural amino acid(s) in a protein.

In one embodiment, the conjugation site is a free or capped thiol group in the side chain of a cysteine, preferably present at a solvent-accessible position in the building block. Cysteine is often the site of choice when it comes to the site-specific modification of proteins, also known as bioconjugation, owing to its favourable properties (nucleophilic profile of the thiol at neutral/near-neutral pH, low natural abundance, general ease of incorporation into proteins via site-directed mutagenesis) (from Spears R. J. et al., “Cysteine protecting groups: applications in peptide and protein science”, Chem. Soc. Rev., 2021, 50, 11098-11155).

In a preferred embodiment, the at least one conjugation site or attachment point is selected from: a thiol group (—SH, free or capped) present in the side chain of a cysteine preferably located at a solvent-accessible position in the protein-based carrier building block, —NH₂ (primary amine, either from the N-terminal end of the protein-based building block or present in the side chain of an amino acid, such as lysine or ornithine), —OH present in the side chain of a tyrosine (either a C-terminal tyrosine, N-terminal tyrosine or a tyrosine preferably present at any other solvent-accessible position in the protein-based carrier building block), C-terminal —COOH and azido group present in the side chain of non-natural amino acids (such as azidolysine). More preferably, the at least one conjugation site or attachment point is a thiol group (free or capped) present in the side chain of a cysteine preferably located at a solvent-accessible position in the protein-based building block.

In one embodiment, the protein-based building block of the present technology comprises six attachment points or conjugation sites, wherein three of them are —SH groups present in the side chain of three Cys, preferably located at solvent-accessible positions, and wherein three of them are —NH₂ present in the side chain of three Lys, preferably located at solvent-accessible positions in the protein-based building block.

Hence, the at least one conjugation site present in the at least one building block comprised in the molecule of the present technology allows for conjugation of different cargos (directly or by means of a linker, as it will be clear to the skilled person and described in detail below). The skilled person is aware of ways of attaching cargos to the conjugation site(s) present in the building block. For instance, Spicer C. D. et al. (“Achieving controlled biomolecule-biomaterial conjugation”, Chem Rev. 2018, 118 (16): 7702-7743), the content of which is herewith incorporated by reference, provides a review on the chemistry of biomolecule conjugation and provide a comprehensive overview of the key strategies for achieving controlled functionalization.

For instance, if a conjugation site is a —SH group (free or capped) present in the side chain of a cysteine preferably located at a solvent-accessible position in the protein-based carrier building block, the cargo can be attached or conjugated to the building block (directly or by means of a linker) by alkylation, metal-assisted arylation, disulphide exchange or addition to a maleimide Michael acceptor. It can also be attached or conjugated using the so-called “PODS-based conjugation, see, e.g., Davydova M. et al., “Synthesis and bioconjugation of thiol-reactive reagents for the creation of site-selectively modified immunoconjugates”, J Vis Exp., 2019, 145:10.3791/59063. These different methods provide a high level of chemoselectivity for cysteine (see, e.g., D. Alvarez Dorta, et al., Chem. Eur. J. 2020, 26, 14257). If at least one conjugation site is a —SH group (free or capped) present in the side chain of a cysteine preferably located at a solvent-accessible position in the protein-based carrier building block, the cargo can be attached or conjugated to it through addition to a maleimide Michael acceptor. Maleimide present in the cargo will specifically react with the at least one free thiol to form a thioether bond, generally at pH 6.5 to 7.5. Of course, if the —SH group is capped or protected, it should first decapped or deprotected (e.g., reduced with reducing reagent, such as dithiothreitol (DTT) or tris(2-carboxyethyl)phosphine (TCEP)), and then the cargo can be attached to it. For instance, an APN-maleimide ‘bifunctional’ linker (see Formula I in the Examples), also known as 3-(4-(2,5-dioxo-2,5-dihydro-1H-pyrrol-1-yl)phenyl)propiolonitrile), can be used to attach or conjugate a cargo to a —SH attachment point present in the side chain of a cysteine preferably located at a solvent-accessible position in the protein-based carrier building block. For instance, a bis-maleimido-PEG3-linker (1,11-bismaleimido-triethyleneglycol) can be used to attach or conjugate a cargo to a —SH attachment point present in the side chain of a cysteine preferably located at a solvent-accessible position in the protein-based carrier building block. In addition, maleimide-modified cargos (see, e.g., PEG-maleimide, N-ethylmaleimide, maleimido-PEG-acid, Resiquimod (R-848)-maleimide, cryptophycin-PEG-maleimide) can be attached to the —SH attachment point present in the side chain of a cysteine preferably located at a solvent-accessible position in the protein-based carrier building block, see also the examples.

For instance, if a conjugation site is a —OH group of a tyrosine preferably located at a solvent-accessible position in the protein-based carrier building block, the cargo can be attached or conjugated to the building block (directly or by means of a linker) by several chemical methods such as cross-linking via catalytic tyrosine mono electronic oxidation, three-component Mannich-type tyrosine conjugation, conjugation via sulphur fluoride exchange chemistry (SuFEx), transition-metal complexes for tyrosine conjugation, diazonium coupling reaction, reactions with triazolinediones, etc. (for a review, see, e.g., D. Alvarez Dorta et al., Chem. Eur. J., 2020, 26, 14257).

Alternatively or additionally, if the conjugation site is the —OH group of an N- and/or C-terminal tyrosine, the cargo can be attached or conjugated to the building block (directly or by means of a linker) enzymatically as described, e.g., in Alan M. Marmelstein et al., Journal of the American Chemical Society, 2020, 142 (11), 5078-5086. As described therein, if conjugation of at least one cargo to a N- and/or C-terminal tyrosine is to be performed, the protein-based building block may preferably be extended with flexible (GG) or (G₄S₁)₁₋₃GG tags (sequences) in order to facilitate the enzymatic addition, as described in Alan M. Marmelstein et al., cited above. In this case, tyrosinase from Agaricus bisporus (abTYR), a copper-dependent enzyme that functions to convert tyrosine into melanin via an o-quinone intermediate, may be used. Alternatively, the much smaller Bacillus megaterium tyrosinase (bmTYR) may be used to catalyze the reaction.

For instance, if the conjugation site is the N-terminal primary amine of the protein-based carrier building block and/or the primary amine present in the side chain of an amino acid preferably located at a solvent-accessible position in the protein-based carrier building block (e.g., Lys, Orn, or any non-natural amino acid with a primary amine on its side chain), the cargo may be attached or conjugated to the carrier building block (directly or by means of a linker) by reaction of a group present in the cargo/linker (e.g., isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, or fluorophenyl esters) and the primary amine. See, e.g., Bioconjugate Techniques (Third edition), 2013, Chapter 3—“The reactions of bioconjugation”, Greg T. Hermanson.

For instance, if the building block comprises at least two conjugation sites which include a —OH from a tyrosine and a —SH from a cysteine preferably located at a solvent-accessible positions in the protein-based carrier building block, thiol nucleophiles can be conveniently capped through disulfide formation with Ellman's reagent. Following the coupling reaction in the —OH from the tyrosine, the thiol groups can be de-capped through brief exposure to an appropriate reducing agent, as described in Alan M. Marmelstein et al., mentioned above.

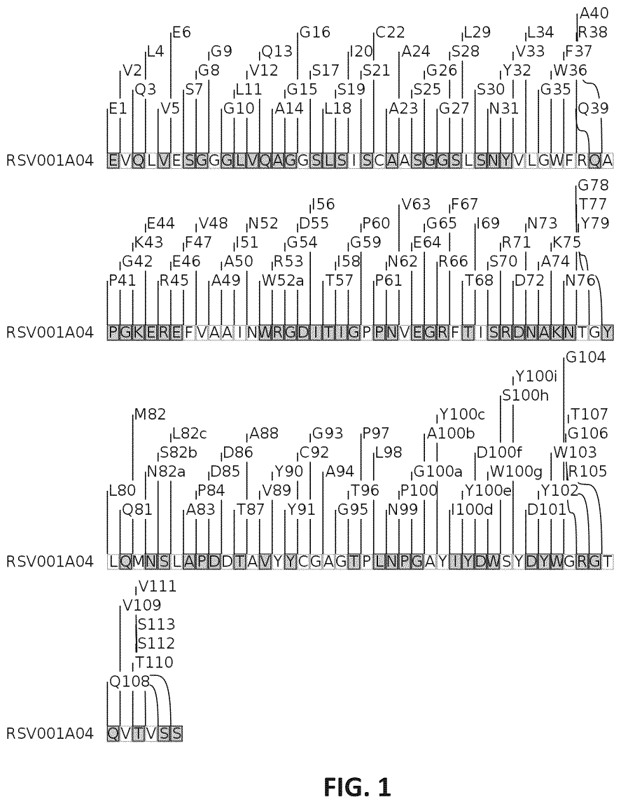

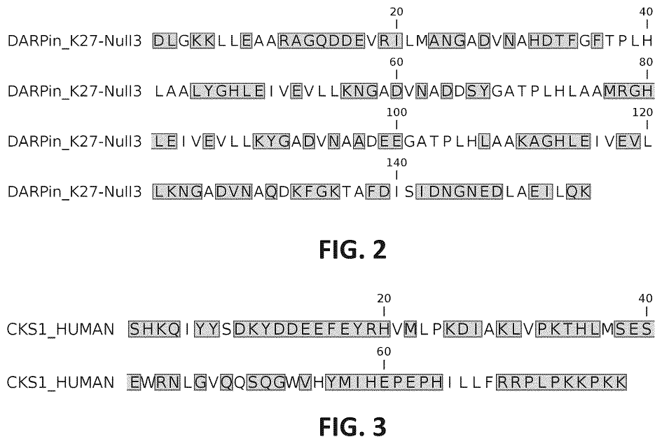

Another option to attach or conjugate a cargo to an attachment point or conjugation site in the protein-based building block (directly or by means of a linker) is the use of sortase-mediated transpeptidation reactions. Sortases allow functionalization of the N-, C-terminus and the creation of non-natural fusions (i.e., N—N or C—C chimeras) via the installation of click handles, see, e.g., Guimaraes C. P. et al. (“Site-specific C-terminal and internal loop labelling of proteins using sortase-mediated reactions”, Nature Protocols, 2013, 8 (9): 1787-1799). As described in this protocol, sortase-mediated reactions are applicable to any protein of interest (e.g., to the protein-based carrier building block of the present technology), provided it contains (i) an LPXTG motif (where X can be any amino acid and glycine cannot be a free carboxylate) as the sortase target or (ii) a suitably exposed glycine residue to serve as the incoming nucleophile. The natural nucleophile of sortase can be replaced by any peptide/protein with an oligoglycine (Gly₁₋₅) at the N-terminus (in many cases a single glycine suffices). In turn, the peptides can be decorated with any cargo molecule (e.g., fluorophores, biotin, cross-linkers, lipids, carbohydrates, nucleic acids), provided that a free N-terminal glycine remains available on the peptide used as the incoming nucleophile. Thus, incubation of sortase, LPXTG-containing protein and nucleophile leads to the covalent attachment of that nucleophile to the protein of interest in a site-specific manner. Guimaraes C. P. et al., mentioned above, provides a protocol that allows the functionalization of any given protein at its C-terminus. The target protein is engineered with a sortase-recognition motif (LPXTG). Upon recognition, sortase cleaves the protein between the threonine and glycine residues, facilitating the attachment of an exogenously added oligoglycine (Glyl-5) peptide modified with the functional group of choice (e.g., the cargo to be attached to the protein-based carrier building block). Theile C. S. et al. (“Site-specific N-terminal labeling of proteins using sortase-mediated reactions”, Nature Protocols, 2013, 8 (9): 1800-1807) describes the use of sortase-mediated reactions to label the N-terminus of any given protein of interest. As described in this protocol, the protein to be labeled is engineered with an exposed stretch of glycines or alanines at its N-terminus when using sortase A from S. aureus or S. pyogenes, respectively. A peptide decorated with a functional group of choice (fluorophores, biotin, lipids, nucleic acids, carbohydrates and so on) and comprising a sortase recognition motif LPXTG/A sequence (X being any amino acid, as stated above) at its C terminus (e.g., the cargo) is then added to the reaction together with sortase. Sortase A cleaves between the threonine and glycine/alanine residues, forming a thioester intermediate with the peptide probe. Nucleophilic attack by the N-terminally modified protein of interest resolves the intermediate, resulting in the formation of a covalent bond between the peptide probe (e.g., the cargo) and the N terminus of the protein (see FIG. 1 of Theile C. S. et al., mentioned above). Alternatively, depsi-peptides can be used for N-terminal labeling, see Theile C. S. et al., mentioned above. Finally, Witte M. D. et al. (“Production of unnaturally linked chimeric proteins using a combination of sortase-catalyzed transpeptidation and click chemistry”, Nature Protocols, 2013, 8 (9): 1808-1819) describes a procedure for the production of N-to-N and C-to-C fusion proteins. By equipping the N-terminus or C-terminus of the proteins of interest with a set of click handles using sortase A, followed by a strain-promoted click reaction, unnatural N-to-N and C-to-C linked (hetero) fusion proteins are established. As described in Witte M. D. et al., peptides for creating C-to-C linked proteins are synthesized with an N-terminal triglycine motif and an azide or cyclooctyne (DIBAC) at the C-terminus (see also FIG. 2 of this document). The proteins of interest are engineered with C-terminal LPXTG sequences. To prepare N-to-N linked proteins, the authors of this protocol synthesize peptides containing the LPXTGG sortase A recognition sequence at the C-terminus (X can be any residue, but the authors prefer a polar residue, such as a glutamic acid, to aid precipitation of the peptide after cleavage from the resin and to increase the solubility of the peptide in water) and an azido or a cyclooctyne group at the N-terminus of the probe. The proteins to be linked should comprise 1-5 Gly at the N-terminus. The final step of the procedure is fusing the click handle-containing proteins, see FIG. 1 of Witte M. D. et al.

In view of the above, it is possible to attach or conjugate cargos to the protein-based carrier building block (directly or by means of a linker) using sortases, as described in detail in Guimaraes C. P. et al., Theile C. S. et al. and Witte M. D. et al., the content of which is incorporated herewith by reference. The cargos may be attached or conjugated (directly or by means of linkers) at conjugation sites or attachment points in the protein-based carrier building block, which are either the N- or C-terminus of the protein-based building block using the above-described sortase methodology. Hence, if the conjugation site or attachment point of the protein-based carrier building block is the C-terminal end of the building block, a cargo may be attached or conjugated to it using sortase, provided that the C-terminal end of the building block comprises a sortase-recognition motif (LPXTG) and the cargo comprises a oligoglycine ((Gly)₁₋₅) modified peptide at the N-terminal (see FIG. 2 of Guimaraes C. P. et al.). If the conjugation site or attachment point of the protein-based carrier building block is the N-terminal end of the building block, a cargo may be attached or conjugated to it using sortase, provided that the N-terminal end of the building block comprises a (Gly)₁₋₅ tag sequence and the cargo comprises a sortase-recognition motif (LPXTG/A) at the C-terminal (see FIG. 1 of Theile C. S. et al.). In addition, protein or peptide cargos can be attached to the N/C-terminal end of the protein-based carrier building block in a N-to-N and/or C-to-C manner, as described in detail in Witte M. D. et al.

Hence, by selecting appropriate (possibly initially capped) conjugation sites, the skilled person is able to attach or conjugate different cargos to the building block.

### Solvent-Accessible Positions

As described above, the at least one conjugation site or attachment point present in the protein-based carrier building block is preferably located at solvent-accessible positions in the building block.

The skilled person is able to identify “solvent-accessible positions” in the carrier building block precursor. This can be performed in silico by means of computer modelling. For instance, the skilled person can make use of readily available software tools such as MAESTRO (Schrödinger, LLC, New York, NY, 2021), a multi-agent prediction system, based on statistical scoring functions (SSFs) and different machine learning approaches, see, e.g., Laimer et al. BMC Bioinformatics (2015) 16:116. In addition, the skilled person can also make use of readily available software tools such as YASARA (www.yasara.org), for identifying at least potential solvent-accessible positions for the at least one conjugation site of the building block. With the help of in silico tools such as MAESTRO or YASARA, the skilled person is able to identify solvent-accessible positions that are potentially suitable for engineering conjugations sites as defined above. Hence, with the help of tools such as MAESTRO or YASARA, potentially suitable conjugation sites are identified. An example of how to identify solvent-accessible positions that are potentially suitable for engineering conjugations sites as defined above is provided in the examples of this application (e.g., Examples 1-3). As described therein, a protein is selected as starting point for developing the protein-based carrier building block (the so-called “building block precursor”). Using, e.g., MAESTRO, residues in the building block precursor with a Solvent-Accessible Surface Area (SASA) greater than or equal to, e.g., 27 Å² (square angstrom) can be considered to be solvent-accessible. The stability (ΔG in solvent) of the mutation of each of the identified residues (e.g., to a cysteine residue) can then be calculated, see, e.g., Laimer J. et al, “MAESTRO—multi agent stability prediction upon point mutations”, BMC Bioinformatics, 2015, 16:116, for further details. Destabilizing mutations (e.g., mutations for which the calculated ΔG in solvent is higher) are generally not further considered as potential positions for conjugation sites or attachment points. Hence, once potentially suitable conjugation sites are identified with the help of tools such as MAESTRO or YASARA, the stability (ΔG in solvent) of the mutation of each of the identified residues (e.g., to a cysteine residue) is calculated. Those residues with lower calculated ΔG in solvent would be preferably further selected as potential positions for conjugation sites or attachment points. For instance, ΔG values in the range of −20 to +5 can be considered as non-destabilizing mutations. The skilled person will understand that the ΔG value for each of the mutations of the identified residues may vary depending on the specific protein and/or the specific mutations considered. The skilled person will also understand that the preferred mutations are those whose ΔG values are the lowest. Depending on these ΔG values, the number of conjugation sites and the type of cargo that will be conjugated, the skilled person will further select certain positions over others among the ones initially identified as potentially solvent-accessible with the help of tools such as MAESTRO or YASARA.

Alternatively or additionally, the skilled person can use hydrogen/deuterium exchange mass spectrometry (HDX-MS) to determine at least potential solvent-accessible positions in a protein. HDX-MS reports on the local chemical environment and solvent accessibility of the protein backbone by monitoring the exchange of peptide bond amide protons with the deuterons of a D20 solvent. The rate of hydrogen-deuterium exchange is dependent on the solvent accessibility and folded state of the protein (see Englander SW. et al., “Hydrogen exchange: the modern legacy of Linderstrøm-Lang”, Protein Sci., 1997, 6 (5): 1101-9).

If the identified solvent-accessible position is to be occupied by a certain amino acid with a reactive group in its side chain, (e.g., by a cysteine), the in silico modelling (e.g., with MAESTRO) will also take into account the potential interactions of the reactive group of that amino acid (e.g., the —SH present in the side chain of the cysteine) with other reactive groups present in the side chain of other amino acids present in the protein-based carrier building block (e.g., with other —SH groups present in the protein, if any).

Additionally or alternatively, the “solvent-accessible positions” can be identified and/or verified empirically. For instance, the “solvent-accessible positions” theoretically identified using available in silico software tools such as MAESTRO, as described above, may preferably be empirically confirmed by manufacturability. Formulation and process stability of potential building block candidates help narrow down lead candidates at an early stage, prior to large-scale manufacturing (see the examples and also, e.g., Ramachander, R., Rathore, N. (2013), “Molecule and manufacturability assessment leading to robust commercial formulation for therapeutic proteins” in: Kolhe, P., Shah, M., Rathore, N. (eds) Sterile Product Development, AAPS Advances in the Pharmaceutical Sciences Series, vol 6. Springer, New York, NY). Hence, once potential suitable solvent-accessible positions have been theoretically identified in the protein-based building block precursor, expression levels, conjugation efficiency, formulation, quality control, solubility, process stability, etc., of the resulting protein-based carrier building block should preferably be evaluated. Solvent-accessible positions which lead to building blocks excelling in expression yield, manufacturability, solubility and/or stability are preferred, see the Examples for further details.

For instance, once suitable solvent-accessible positions have been theoretically identified in the building block precursor, protein expression of the selected variants (i.e., the resulting protein-based building blocks with amino acid(s) bearing the conjugation site(s) in the theoretically-selected solvent-accessible position(s)) may take place. In this step it can be asserted whether the introduction of the specific amino acids at the theoretically-identified solvent accessible positions (e.g., point mutations, addition of amino acids at the N- and/or C-terminal of the protein, etc.) has a negative impact on, e.g., the synthesis, expression levels, conjugation efficiency or 3D globular structure of each specific variant. In addition, the minimal required solubility and lack of specific binding to human proteins (and, optionally, to non-protein molecules and/or non-human proteins, preferably to the precursor's target), as described in detail above, can be assessed. Possible changes in 3D structure could be assessed, for example, by CD (circular dichroism) spectrum analysis, as described in detail above. In addition, the stability of the resulting variants can also be confirmed with a Thermal Shift Assay. This assay detects protein melting temperatures (Tm) and can thus be used to check protein stability. It can be used to characterize the stability/folding of a protein's 3D structure. SYPRO® Orange is a naturally quenched dye that interacts with the hydrophobic core of proteins which becomes visible following thermal denaturation. As a result, the temperature in the middle of the thermal denaturation process is labelled as melting temperature Tm. This is a way of assessing the stability of the resulting variants or mutants.

In addition, “model cargos” can be attached or conjugated to the selected variants, in order to quantify the extent of conjugation (conjugation efficiency), i.e., to ascertain whether the resulting protein-based building block with the conjugation sites at the selected solvent-accessible positions will in practice be suitable for the attachment or conjugation of the desired cargos. A “model cargo” may be any molecule with a molecular weight higher than, e.g., 100 Da. For instance, if a potential conjugation site is a thiol group, a “model cargo” may be a maleimide-modified alanine (e.g., N-Maleoyl-β-alanine), or a biotin-maleimide, as described in Junutula, J. et al. (“Site-specific conjugation of a cytotoxic drug to an antibody improves the therapeutic index”, Nat Biotechnol, 26, 925-932 (2008)). For instance, if the conjugation of the “model cargo(s)” results in a stable conjugate (protein-based building block with one or more model cargos conjugated to it), with an acceptable extent of conjugation (to be decided on a case-by-case basis, for example ≥90% conjugation efficiency, such as 90% conjugation efficiency, or 95% conjugation efficiency, or 97% conjugation efficiency, or 99% conjugation efficiency or more), allowing a standard PK in vivo, preserving its globular 3D structure and the conjugation status in vivo, etc., those solvent-accessible positions should be preferred for cargo conjugation, and conjugation of the desired cargo(s) may take place, see also the examples below.

### Point Mutations

In one embodiment, the at least one conjugation site present in the building block may be generated by introducing specific point mutations at solvent-accessible positions in the peptide sequence of the building block precursor. For instance, point mutations may be introduced at solvent-accessible positions in the building block precursor in order to generate the protein-based building block comprised in the molecule of the present technology, which comprises at least one conjugation site or attachment point at defined solvent-accessible positions, as described herein.

For instance, the conjugation sites may be generated by mutating specific amino acids preferably at solvent-accessible positions of a building block precursor to cysteine (“Cys-mutations”). Alternatively or additionally, the conjugation sites may be generated by mutating specific amino acids preferably at solvent-accessible positions of a building block precursor to natural or non-natural amino acids with a reactive group in its side chain. Amino acid distribution data of occurrence at certain positions (e.g., Cys, Ser) in the building block precursor can also be used to guide the design and introduction of conjugation sites.

Additionally or alternatively, the building block precursor may be modified by adding one or more amino acids at the N- and/or C-terminal of the protein sequence, to introduce at least one conjugation site or attachment point preferably at a solvent-accessible position, as described herein, to generate the protein-based building block of the present technology.

In another embodiment, the at least one conjugation site may be already present preferably at solvent-accessible positions in the protein-based building block precursor, and there is no need of generating it. This is the case for the primary amine at the N-terminal of the building block, the —COOH at the C-terminal or in the side chain of the building block, the primary amine in the side chain of, e.g., a lysine preferably already present at a solvent-accessible position in the building block precursor or the thiol group in the side chain of a cysteine preferably already present at a solvent-accessible position in the building block precursor.

If the building block comprises more than one conjugation sites, these can be generated by introducing, e.g., specific point mutations preferably at solvent-accessible positions in the peptide sequence of the building block precursor. Additionally or alternatively, other suitable conjugation sites or attachment points may be already present preferably at solvent-accessible positions in the building bock precursor, i.e., there is no need of generating these conjugation sites by introducing, e.g., specific point mutations and/or adding one or more amino acids at the N- and/or C-terminal of the building bock precursor. The skilled person will decide on the number and position of the attachment point(s) or conjugation site(s) based on the protein-based building block and the cargo(s) to be attached to it, directly or by means of a linker, as described herein.

As described in detail above, preferably, the point mutations are non-destabilizing point mutations. Stability of mutants can be calculated with different methods which predict the impact of mutations on protein stability, e.g., based on artificial intelligence (AI). For instance, stability of mutants can be calculated with MAESTRO, as defined above and explained in detail in the examples, and can also be confirmed empirically by manufacturability (including but not limited to expression level and stability assessment, as described above).

In a preferred embodiment, the point mutations are mutations of amino acids preferably located at solvent-accessible positions in the building block precursor to cysteines. In another embodiment, the point mutation consists of the replacement of a serine residue preferably in a solvent-accessible position of the building block precursors by a cysteine. In another embodiment, the point mutations are mutations of preferably solvent-accessible amino acids in the building block precursor to lysines. In another embodiment, the point mutations are mutations of preferably solvent-accessible amino acids in the building block precursor to tyrosines. In another embodiment, the point mutations are mutations of preferably solvent-accessible amino acids in the building block precursor to a natural or non-natural amino acid, as described above.

Addition of a C- or N-Natural and/or Non-Natural Amino Acid with a Reactive Group in its Side Chain

For instance, the conjugation sites may be generated by adding, in the building block precursor, one or more C- or N-terminal natural and/or one or more C- or N-terminal non-natural amino acid(s) with a reactive group in its side chain. Preferably, if present, the one or more terminal natural or non-natural amino acid is added at the C-terminus of the building block precursor. For instance, one or more of the conjugation sites is (are) generated by adding a N- or C-terminal cysteine, a N- or C-terminal tyrosine and/or a N- or C-terminal non-natural amino acid to the protein-based building block precursor. Preferably, at least one of the conjugation sites is generated by adding a N- or C-terminal tyrosine to the protein-based building block precursor, preferably a C-terminal tyrosine. In a preferred embodiment, the N- and/or C-terminal Tyr is preceded/followed by flexible (GG) or ((G₄S₁)₁₋₃GG) sequences (e.g. -GGY, -(G₄S₁)₁₋₃GGY, YGG-, Y(G₄S₁)₁₋₃GG-, YGG(S₁G₄)₁₋₃-, or YGG(G₄S₁)₁₋₃-), as described in detail in Alan M. Marmelstein et al., Journal of the American Chemical Society, 2020, 142 (11), 5078-5086.

Hence, the at least one protein-based carrier building block comprised in the molecule of the present technology may comprise a N- and/or C-terminal Cys, Tyr, and/or non-natural amino acid, for instance a C-terminal Tyr, as in a -GGY or -(G₄S₁)₁₋₃GGY tag (sequence).

In addition, the at least one protein-based carrier building block of the present technology may comprise a N- and/or C-terminal conjugation site or attachment point suitable for conjugation with sortase, as described above. In these cases, the protein-based carrier building block should be engineered to comprise a C-terminal sortase recognition motif (LPXTG, where X can be any amino acid), a N-terminal polygly ((Gly)₁₋₅) tag or both. In addition, if N-to-N and/or C-to-C attachments or conjugations are desired, the protein-based carrier building block should be engineered to comprise a C-terminal sortase recognition motif (for C-to-C attachments) or a N-terminal polygly ((Gly)₁₋₅) tag (for N-to-N attachments), as described in detail above. See in particular Guimaraes C. P. et al., Theile C. S. et al. and Witte M. D. et al., listed above.

Finally, as described above, the conjugation sites may be generated by combinations of the above mechanisms, e.g., the at least one conjugation site can be obtained by performing point mutations (e.g., Ser to Cys at a solvent-accessible position of the building block, as described above), or by adding a C- and/or N-terminal amino acid, such as cysteine, or tyrosine, or a non-natural amino acid, or a sortase recognition motif, or a polygly ((Gly)₁₋₅) tag to the protein-based building block precursor, as described above.

### Examples of Building Blocks

### Small Globular Non-Human Proteins-Based Building Blocks

The protein-based carrier building block(s) of the present technology may be based on a small globular non-human protein. In the context of the present technology, a “small globular non-human protein” refers to a non-human protein which has a size (molecular mass) of about 2.5 to about 70 kDa, preferably of about 2.5 to about 50 kDa, such as about 2.5 to less than 50 kDa, more preferably of about 2.5 to about 30 kDa, even more preferably of about 2.5 to about 16 kDa, as described herein and which has a globular three-dimensional (3D) structure, as described herein. In addition, the at least one non-human protein-based carrier building block does not specifically bind to any human protein, as defined in this specification, preferably it also does not specifically bind to any non-protein molecule (such as nucleic acids (e.g., DNA, RNA), glycans, lipids (e.g., such as phosphatidylserine (PS)), etc.), such as any human non-protein molecule (biomolecule) (such as human DNA, human RNA, human glycans, human lipids (e.g., such as phosphatidylserine (PS)), etc.), preferably it also does not specifically bind to any non-protein molecule (such as nucleic acids (DNA, RNA), glycans, lipids (e.g., such as phosphatidylserine (PS)), etc.), to which the building block precursor binds specifically, if any, and preferably it also does not specifically bind to any non-human protein (e.g., a bacterial and/or viral protein) to which the building block precursor binds specifically, if any. Further, preferably, the at least one non-human protein-based carrier building block (i) does not specifically bind to any human cell and/or cell type, or binds to a human cell and/or cell type with a K_{D} (K_{D} value) greater than 5×10⁻⁴ mol/litre, preferably as determined by cell-binding assay, (ii) does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein, and/or (iii) does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein.

In a preferred embodiment, the protein-based carrier building block does not specifically bind any non-human protein and/or non-protein molecule, preferably the precursor's target, when a cargo is conjugated to the at least one attachment point or conjugation site on the protein-based carrier building block, as described above. Hence, in a preferred embodiment, the molecule of the present technology, which comprises at least one protein-based building block and at least one cargo attached to the at least one protein-based building block through the at least one conjugation site or attachment point, does not specifically bind any non-human protein or non-protein molecule, such as any human non-protein molecule, as described herein, in particular it does not specifically bind any protein or non-protein molecule to which the building block precursor binds, if any.

As described above, in the context of the present technology, if the protein-based building block or molecule of the present technology shows any interaction with one or more human protein (or non-human protein, or non-protein molecule, as described above), such interaction is characterized by low specificity and/or low affinity, as described in detail above.

A human protein is a protein present in the human body. The Human Protein Atlas (HPA, https://www.proteinatlas.org) is a Swedish-based program initiated in 2003 with the aim to map all the human proteins in cells, tissues, and organs.

Small globular non-human proteins, in the context of the present technology, include proteins which are derived from human proteins, but which have been modified so that they are no longer human proteins. Examples of small globular non-human proteins are ISVDs, such as “human ISVDs” (e.g., V_{H}, V_{L}) and “non-human ISVDs” (e.g., V_{HH}, non-human V_{H}, V_{L} or engineered ISVDs), DARPins (derived from ankyrin repeat proteins), affibodies or affitins.

The small globular non-human proteins may have a therapeutic or targeting activity.

### Immunoglobulin Single Variable Domains (ISVDs)-Based Building Blocks

In one embodiment, the at least one protein-based carrier building block of the present technology is based on a polypeptide which comprises or, alternatively, consists of, at least one immunoglobulin single variable domain (ISVD), such as an ISVD derived from V_{H} or V_{HH} (a heavy-chain ISVD).

As described above, the protein-based carrier building block of the present technology has a globular 3D structure, is soluble, has a size (molecular mass) of about 2.5 to about 70 kDa, such as about 2.5 to about 50 kDa, such as about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa. In addition, the at least one building block comprised in the molecule of the present technology does not specifically bind to any human protein, as defined in this specification, preferably it also does not specifically bind to any non-protein molecule (such as DNA, RNA, glycans, lipids (e.g., such as phosphatidylserine (PS)), etc.), such as any human non-protein molecule (such as human DNA, human RNA, human glycans, human lipids (e.g., such as phosphatidylserine (PS)), etc.), preferably it also does not specifically bind to any non-protein molecule (such as DNA, RNA, glycans, lipids (e.g., such as phosphatidylserine (PS)), etc.), to which the building block precursor binds specifically, if any, and preferably it also does not specifically bind to any non-human protein (e.g., a bacterial and/or viral protein) to which the building block precursor binds specifically, if any.

In a preferred embodiment, the protein-based carrier building block does not specifically bind any non-human protein and/or non-protein molecule, preferably the precursor's target, when a cargo is conjugated to the at least one attachment point or conjugation site on the protein-based carrier building block, as described above. Hence, in a preferred embodiment, the molecule of the present technology, which comprises at least one protein-based building block and at least one cargo attached to the at least one protein-based building block through the at least one conjugation site or attachment point, does not specifically bind any non-human protein or non-protein molecule, such as any human non-protein molecule, as described herein, in particular it does not specifically bind any protein or non-protein molecule to which the building block precursor binds, if any.

As described above, in the context of the present technology, if the protein-based building block or molecule of the present technology shows any interaction with one or more human protein (or non-human protein, or non-protein molecule, as described above), such interaction is characterized by low specificity and/or low affinity, as described in detail above.

Hence, in the specific embodiment where the at least one protein-based building block is based on an ISVD, preferably a heavy-chain ISVD, the resulting ISVD-based building block does not specifically bind to any human protein. In addition, as explained above, it is preferred that the ISVD-based building block does not specifically bind to any non-protein molecule, such as any human non-protein molecule. Furthermore, it is also preferred that the ISVD-based building block does not specifically bind to any non-human protein or non-protein molecule to which the protein-based carrier building block precursor specifically binds, if any, as described above.

In the context of the present technology, an “ISVD-based building block” refers to a protein-based building block which derives from an ISVD, i.e., which is structurally similar to an ISVD but does not specifically bind to any human protein, preferably does not specifically bind to any target to which the ISVD specifically binds. For instance, the ISVD-based building block has a sequence identity of at least 60%, or 70%, or 80% with an ISVD, e.g., with its ISVD precursor. For instance, the ISVD-based building block has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with an ISVD, e.g., with its ISVD precursor. For instance, an ISVD-based building block may share the whole amino acid sequence with its ISVD precursor with the exception of at least one, such as one, two, three, four, five, six, seven, eight, nine, ten, fifteen, eighteen, twenty, twenty-five, thirty or more amino acids. In addition, the ISVD-based building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind any human protein and preferably does not specifically bind any protein or non-protein molecule to which the precursor specifically binds.

The skilled person is aware of means for eliminating the specific binding properties of a certain ISVD precursor, e.g., by performing mutations in the amino acids responsible for the binding of the ISVD to the target (e.g., in one or more of the amino acids conforming the CDRs of the ISVD), by adding amino acids and/or by deleting amino acids from the precursor's sequence.

The term “immunoglobulin single variable domain” (ISVD), interchangeably used with “single variable domain”, defines immunoglobulin molecules wherein the antigen binding site is present on, and formed by, a single immunoglobulin domain. This sets ISVDs apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an ISVD as, in these cases, binding to the respective epitope of an antigen would normally not occur by one single immunoglobulin domain but by a pair of associating immunoglobulin domains such as light and heavy chain variable domains, i.e., by a V_{H}-V_{L} pair of immunoglobulin domains, which jointly bind to an epitope of the respective antigen.

In contrast, generally, ISVDs are capable of specifically binding to an epitope of the antigen without pairing with an additional immunoglobulin variable domain. The binding site of an ISVD is formed by a single V_{H}, a single V_{HH} or single V_{L} domain.

In the context of the present technology, in the specific embodiment where the at least one protein-based building block is based on an ISVD, the ISVD building block precursor may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as the resulting building block has a globular 3D structure, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and is soluble, as defined in detail above. An ISVD which may preferably be the precursor of the protein-based building block comprised in the molecule of the present technology can for example be a heavy chain ISVD, such as a V_{H}, V_{HH}, including a camelized V_{H} or humanized V_{HH}. In one embodiment, the protein-based building block precursor is a V_{HH}, including a camelized V_{H} or humanized V_{HH}, as long as the resulting protein-based building block is soluble, has a globular 3D structure, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to human proteins. In addition, preferably, the resulting building block does not specifically bind to any non-protein molecule, such as DNA, RNA, lipids (e.g., such as phosphatidylserine (PS)) or glycans, e.g., glycoplipids. Furthermore, preferably, the resulting building block does also not specifically bind to any non-human protein to which the protein-based carrier building block precursor specifically binds, if any, as described above. Heavy chain ISVDs can be derived from a conventional four-chain antibody or from a heavy chain antibody.

For example, the ISVD precursor may be a single domain antibody (or an amino acid sequence that is suitable for use as a single domain antibody), a “dAb” or dAb (or an amino acid sequence that is suitable for use as a dAb) or a Nanobody® ISVD (as defined herein, and including but not limited to a V_{HH}); other single variable domains, or any suitable fragment of any one thereof, as long as the resulting protein-based building block is soluble, has a globular 3D structure and does not specifically bind to human proteins, preferably does not specifically bind to any non-protein (human) molecule, such as DNA, RNA, lipids (e.g., such as phosphatidylserine (PS)) or glycans, e.g., glycoplipids, and, preferably, does also not specifically bind to any non-human protein to which the protein-based carrier building block precursor specifically binds, if any, as described above.

Preferably, the ISVD precursor is a V_{H}, a humanized V_{H}, a human V_{H}, a V_{HH}, a humanized V_{HH} or a camelized V_{H}. More preferably, the ISVD precursor is a Nanobody® ISVD (such as a V_{HH}, including a humanized V_{HH} or camelized V_{H}) or a suitable fragment thereof, as long as the protein-based building block is soluble, has a globular 3D structure and does not specifically bind to human proteins, preferably does not specifically bind to any non-protein (human) molecule, such as DNA, RNA, lipids (e.g., such as phosphatidylserine (PS)) or glycans, e.g., glycoplipids, and, preferably, does also not specifically bind to any non-human protein to which the protein-based carrier building block precursor specifically binds, if any, as described above. Nanobody® is a registered trademark from Ablynx N.V.

“V_{HH} domains”, also known as V_{HH}s, V_{HH} antibody fragments, and V_{HH} antibodies, have originally been described as the antigen binding immunoglobulin variable domain of “heavy chain antibodies”; i.e., of “antibodies devoid of light chains”, see Hamers-Casterman et al., Nature, 363:446-448, 1993. The term “V_{HH} domain” has been chosen in order to distinguish these variable domains from the heavy chain variable domains that are present in conventional 4-chain antibodies, which are referred to herein as “V_{H} domains”, and from the light chain variable domains that are present in conventional 4-chain antibodies, which are referred to herein as “Vi domains”. For a further description of V_{HH}'s, reference is made to the review article by Muyldermans (“Single domain camel antibodies: current status”, J Biotechnol., 2001, 74:277-302). V_{HH} domains can be obtained from heavy chain-only antibodies (HCAbs) that are circulating in Camelidae, see e.g., Muyldermans S., “A guide to: generation and design of nanobodies”, FEBS J., 2021, 288 (7): 2084-2102. Hence, in a preferred embodiment, the ISVD-based building block has a sequence identity of at least 80% with a V_{HH} (such as a humanized V_{HH} or camelized V_{H}), e.g., its V_{HH} precursor. For instance, the ISVD-based building block has a sequence identity of at least 60%, or at least 70%, or 80%, or at least 85%, such as at least 90%, such as at least 91%, such as at least 92%, such as at least 93%, such as at least 94%, such as at least 95%, such as at least 96%, such as at least 97%, such as at least 98%, such as at least 99%, or more with a V_{HH}, e.g., its V_{HH} precursor. For instance, the ISVD-based building block may share the whole amino acid sequence with its V_{HH} precursor with the exception of at least one, such as one, two, three, four, five, six, seven, eight, nine, ten, fifteen, eighteen, twenty, twenty-five, thirty or more amino acids, which are different in the protein-based carrier building block.

Typically, the generation of immunoglobulins involves the immunization of experimental animals, fusion of immunoglobulin producing cells to create hybridomas and screening for the desired specificities. Alternatively, immunoglobulins can be generated by screening of naïve, immune, or synthetic libraries, e.g., by phage display.

The generation of immunoglobulin sequences, such as VHHs, has been described extensively in various publications, among which WO 94/04678, Hamers-Casterman et al. 1993 (“Naturally occurring antibodies devoid of light chains”, Nature, 363:446-448, 1993) and Muyldermans et al. 2001 (“Single domain camel antibodies: current status”, J Biotechnol., 2001, 74:277-302) can be exemplified. In these methods, camelids are immunized with the target antigen in order to induce an immune response against said target antigen. The repertoire of VHHS obtained from said immunization is further screened for VHHS that bind (or not) a target antigen.

In the context of the present technology, immunoglobulin sequences of different origin may be used, comprising mouse, rat, rabbit, donkey, human and camelid immunoglobulin sequences. In the context of the present technology, fully human, humanized or chimeric sequences are also included. In the context of the present technology, camelid immunoglobulin sequences and humanized camelid immunoglobulin sequences, or camelized domain antibodies, e.g. camelized dAb as described by Ward et al. (Nature, 341:544, 1989) (see for example WO 94/04678 and Davies and Riechmann, “‘Camelising’ human antibody fragments: NMR studies on VH domains”, Febs Lett., 339:285-290, 1994 and “Single antibody domains as small recognition units: design and in vitro antigen selection of camelized, human VH domains with improved protein stability”, Prot. Eng., 1996, 9 (6): 531-537) are also included. A “humanized V_{HH}” comprises an amino acid sequence that corresponds to the amino acid sequence of a naturally occurring V_{HH} domain, but that has been “humanized”, i.e., by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the further description herein and the prior art (e.g., WO 2008/020079). Again, it should be noted that such humanized VHHS can be obtained in any suitable manner known per se and thus are not strictly limited to polypeptides that have been obtained using a polypeptide that comprises a naturally occurring V_{HH} domain as a starting material. Preferably, if the building block of the present technology is a V_{HH}, the V_{HH} is a humanized V_{HH}.

A “camelized V_{H}” comprises an amino acid sequence that corresponds to the amino acid sequence of a naturally occurring V_{H} domain, but that has been “camelized”, i.e., by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g. WO 2008/020079). Such “camelizing” substitutions are usually inserted at amino acid positions that form and/or are present at the V_{H}-V_{L} interface, and/or at the so-called Camelidae hallmark residues, as defined herein (see for example WO 94/04678 and Davies and Riechmann, 1994 and 1996, supra). In one embodiment, the V_{H} sequence that is used as a starting material or starting point for generating or designing the camelized V_{H} is a V_{H} sequence from a mammal, or the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an ISVD sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

Also, as further described in paragraph q) on pages 58 and 59 of WO 2008/020079, the amino acid residues of an ISVD are numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods, 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application CDR sequences may also be described according to Kabat numbering with AbM CDR annotation, as described in Kontermann and Dübel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 10³-113.

Determination of CDR regions may also be done according to different methods. In the CDR determination according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are a suitable combination of immunoglobulin framework sequences or framework sequences that have been derived from immunoglobulin framework sequences, for example by humanization or camelization. For example, the framework sequences may be framework sequences derived from a light chain variable domain (e.g. a V_{L}-sequence) and/or from a heavy chain variable domain (e.g. a V_{H}-sequence or V_{HH} sequence). In one aspect, the framework sequences are either framework sequences that have been derived from a V_{HH}-sequence in which said framework sequences may optionally have been partially or fully humanized or are conventional V_{H} sequences that have been camelized (as defined herein).

In particular, the framework sequences present in the ISVD sequences referred to in the present technology may contain one or more of Hallmark residues (as defined herein), such that the ISVD sequence is a Nanobody® ISVD, such as, e.g., a V_{HH}, including a humanized V_{HH} or camelized V_{H}. Some non-limiting examples of suitable combinations of such framework sequences will become clear from the further disclosure herein.

However, it should be noted that, in the context of the present technology, the origin of the ISVD sequence or the origin of the nucleotide sequence used to express it is not limited, nor as to the way that the ISVD sequence or nucleotide sequence is or has been generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template, e.g., DNA or RNA isolated from a cell, nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, the ISVD precursor is preferably a V_{HH}, including a humanized V_{HH} or camelized V_{H}, or a suitable fragment thereof, more preferably a humanized V_{HH} or a suitable fragment thereof. The resulting protein-based building block should be soluble, have a globular 3D structure and not specifically bind to human proteins, preferably should also not specifically bind to any non-protein molecule and preferably should also not specifically bind to any non-human protein to which the V_{HH} precursor specifically binds, if any, as described above. Preferably, as described above, the molecule comprising at least one V_{HH} (including humanized V_{HH} or camelized V_{H})-derived protein-based building block and at least one cargo attached to it through the at least one conjugation site or attachment point, does not specifically bind to any non-protein molecule and/or does not specifically bind to any non-human protein to which the V_{HH} (including humanized V_{HH} or camelized V_{H}) precursor specifically binds.

Further, preferably, the at least one ISVD-based carrier building block (i) does not specifically bind to any human cell and/or cell type, or binds to a human cell and/or cell type with a K_{D} (K_{D} value) greater than 5×10⁻⁴ mol/litre, preferably as determined by cell-binding assay, (ii) does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein, and/or (iii) does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein.

As described above, the ISVD precursor is preferably a VHH, humanized VHH or camelized VH, such as a Nanobody® ISVD, or a suitable fragment thereof, more preferably a humanized Nanobody® ISVD or a suitable fragment thereof. The resulting protein-based building block should be soluble, have a globular 3D structure and not specifically bind to human proteins, preferably should also not specifically bind to any non-protein molecule and preferably should also not specifically bind to any non-human protein to which the VHH, humanized VHH or camelized VH, such as Nanobody® ISVD, precursor specifically binds, if any, as described above. Preferably, as described above, the molecule comprising at least one VHH, humanized VHH or camelized VH, such as Nanobody® ISVD-derived protein-based building block and at least one cargo attached to it through the at least one conjugation site or attachment point, does not specifically bind to any non-protein molecule and/or does not specifically bind to any non-human protein to which the VHH, humanized VHH or camelized VH, such as Nanobody® ISVD precursor specifically binds. For a general description of Nanobody® ISVDs, reference is made to the present description, as well as to the prior art cited herein. In this respect, it should however be noted that this description and the prior art mainly described Nanobody® ISVDs of the so-called “V_{H}3 class”, i.e. Nanobody® ISVDs with a high degree of sequence homology to human germline sequences of the V_{H}3 class such as DP-47, DP-51 or DP-29. It should however be noted that the present technology in its broadest sense can generally use any type of Nanobody® ISVD, and for example also uses the Nanobody® ISVDs belonging to the so-called “V_{H}4 class”, i.e. Nanobody® ISVDs with a high degree of sequence homology to human germline sequences of the V_{H}4 class such as DP-78, as for example described in WO 2007/118670.

In one embodiment, the at least one protein-based carrier building block comprised in the molecule of the present technology is derived from a Nanobody® ISVD belonging to the so-called “V_{H}3 class”, i.e. a Nanobody® ISVDs with a high degree of sequence homology to human germline sequences of the V_{H}3 class such as DP-47, DP-51 or DP-29, as long as the protein-based building block is soluble, has a globular 3D structure and does not specifically bind to human proteins, preferably does not specifically bind to any non-protein human molecule and preferably does also not specifically bind to any non-human protein to which the ISVD precursor specifically binds, as described above.

Generally, Nanobody® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Generally, a Nanobody® ISVD can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:

one or more of the amino acid residues at positions 11, 37, 44, 45, 47, 83, 84, 103, 104 and 108 according to the Kabat numbering are chosen from the Hallmark residues mentioned in Table 3 below.

TABLE 3
Hallmark Residues in Nanobody ® ISVDs (according to Kabat numbering)

| Position | Human V_{H}3 | Hallmark Residues |
|---|---|---|
| 11 | L, V; predominantly L | L, S, V, M, W, F, T, Q, E, A, R, G, K, Y, N, P, I; preferably |
| | | L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾. |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾ , L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; preferably |
| | | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
⁽¹⁾In particular, but not exclusively, in combination with KERE or KORE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

Thus, a Nanobody® ISVD can be defined as an amino acid sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are chosen from the Hallmark residues mentioned in Table 3.

For instance, when the protein-based building block of the present technology is based on an ISVD, it may derive from anti-viral ISVDs, such as from anti-viral V_{HH} or Nanobody® ISVDs. For instance, the building block of the present technology may derive from a functional ISVD (i.e., an ISVD which specifically binds to human proteins, and/or to non-human proteins, such as viral proteins and/or bacterial proteins, and/or to non-protein molecules, such as human non-protein molecules) which has been engineered/modified so that it no longer specifically binds to any human protein, preferably which has been engineered/modified so that it also no longer specifically binds to any non-human protein to which it originally bound, such as bacterial and/or viral proteins, as described in detail above and/or preferably which has been engineered/modified so that it also no longer specifically binds to any non-protein molecule to which it originally bound, if any. In a further preferred embodiment, the ISVD-based building block of the present technology derives from an ISVD, such as from a heavy-chain ISVD, preferably from a Nanobody® ISVD, which has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) may be Val or Leu, preferably Val; and/or the amino acid at position 89 (according to Kabat) may be preferably Val, Thr or Leu, preferably Leu; and/or the amino acid at position 110 (according to Kabat) may be preferably Thr, Lys or Gln, preferably Thr; and/or the amino acid at position 112 (according to Kabat) may be Ser, Lys or Gln, preferably Ser; and/or the ISVD-based building block may contain a C-terminal extension of 1-5 amino acids chosen from any naturally occurring amino acid.

The resulting ISVD-based building block may be derived from a variant from an anti-hRSV ISVD, such as, e.g., variants from the anti-hRSV ISVDs depicted on Table A-2 starting on p. 69 of WO 2018/099968. In a preferred embodiment, the resulting ISVD-based building block is derived from a variant from ISVD RSV001A04, SEQ ID NO.: 179 in the present description, and also referred to as RSV001A04, and described in detail in SEQ ID NO.: 5 on Table A-1, page 388 of WO 2010/139808 (referred therein to as NC41)). In this specific embodiment, the protein-based carrier building block derived from RSV001A04 does not specifically bind any human protein. In this embodiment, the “building block precursor” (or “ISVD precursor”) is RSV001A04, SEQ ID NO.: 179:

EVQLVESGGGLVQAGGSLSISCAASGGSLSNYVLGWFRQAPGKEREFVA
AINWRGDITIGPPNVEGRFTISRDNAKNTGYLQMNSLAPDDTAVYYCGA
GTPLNPGAYIYDWSYDYWGRGTQVTVSS.

Once an ISVD is selected as starting point (as “ISVD precursor”, see above), residues preferably located at solvent-accessible positions should be identified to generate the at least one conjugation site, as described in detailed above in this description. Additionally or alternatively, the conjugation site(s) may already be in the ISVD precursor, either as reactive groups in the side chain of amino acids preferably located at solvent-accessible positions or as free N-terminal primary amine and/or free C-terminal carboxylic acid.

For instance, one or more of the identified residues, preferably located at solvent-accessible positions in the amino acid sequence of the ISVD precursor are replaced by a cysteine, a lysine, a tyrosine and/or a non-natural amino acid.

In one embodiment, the at least one protein-based building block comprised in the molecule of the present technology is derived from an ISVD, such as an ISVD belonging to the so-called “V_{H}3 class”, wherein the resulting building block comprises at least one cysteine, at least one lysine, at least one non-natural amino acid and/or at least one tyrosine, preferably located at one or more solvent-accessible positions. In another embodiment, the at least one protein-based building block comprised in the molecule of the present technology is derived from an ISVD, such as an ISVD belonging to the so-called “V_{H}3 class”, wherein the resulting building block comprises at least one engineered cysteine, at least one engineered lysine, at least one non-natural amino acid and/or at least one engineered tyrosine, preferably located at one or more solvent-accessible positions.

Preferably, the building block of the molecule of the present technology, when it is derived from an ISVD, preferably from a V_{HH} (including humanized V_{HH} or camelized V_{H}) or Nanobody® ISVD, as described above, comprises a leucine at position 108 (according to Kabat numbering). In other embodiments, the building block of the molecule of the present technology, when it is derived from an ISVD, as described above, comprises a valine at position 11, a leucine at position 89 and/or a leucine at position 108 (according to Kabat numbering).

In one embodiment, the at least one protein-based carrier building block present in the molecule of the present technology comprises, or alternatively, consists of, SEQ ID NO.: 186:

X₁VX₂LX₃EX₄X₅GX₆X₇X₈X₉X₁₀X₁₁GX₁₂X₁₃X₁₄₁X₁₅CX₁₆AX₁₇X₁₈X₁₉X₂₀L
X₂₁X₂₂X₂₃VLGWFRX₂₄AX₂₅X₂₆X₂₇X₂₈X₂₉X₃₀FVAAINX₃₁X₃₂X₃₃X₃₄X₃₅
X₃₆X₃₇X₃₈PX₃₉X₄₀VX₄₁X₄₂X₄₃FX₄₄₁X₄₅X₄₆X₄₇X₄₈X₄₉X₅₀X₅₁TGX₅₂LX₅₃M
X₅₄X₅₅LX₅₆X₅₇X₅₈DX₅₉AX₆₀YX₆₁CGAGX₆₂PX₆₃X₆₄X₆₅X₆₆AYX₆₇X₆₈X₆₉
X₇₀SYX₇₁X₇₂X₇₃GX₇₄X₇₅TX₇₆VX₇₇VX₇₈X₇₉X₈₀X₈₁X₈₂,

wherein
X₁ (position 1 according to Kabat numbering) can be Glu or any amino acid with a reactive group in its side chain, such as cysteine;
X₂ (position 3 according to Kabat numbering) can be Gln or any amino acid with a reactive group in its side chain, such as cysteine;
X₃ (position 5 according to Kabat numbering) can be Val or any amino acid with a reactive group in its side chain, such as cysteine;
X₄ (position 7 according to Kabat numbering) can be Ser or any amino acid with a reactive group in its side chain, such as cysteine;
X₅ (position 8 according to Kabat numbering) can be Gly or any amino acid with a reactive group in its side chain, such as cysteine;
X₆ (position 10 according to Kabat numbering) can be Gly or any amino acid with a reactive group in its side chain, such as cysteine;
X₇ (position 11 according to Kabat numbering) can be Leu, Val Ser, Met, Trp, Phe, Thr, Gln, Glu, Ala, Arg, Gly, Lys, Tyr, Asn, Pro or Ile, preferably Leu or Val or any amino acid with a reactive group in its side chain, such as cysteine;
X₈ (position 12 according to Kabat numbering) can be Val or any amino acid with a reactive group in its side chain, such as cysteine;
X₉ (position 13 according to Kabat numbering) can be Gln or any amino acid with a reactive group in its side chain, such as cysteine;
X₁₀ (position 14 according to Kabat numbering) can be Ala or any amino acid with a reactive group in its side chain, such as cysteine;
X₁₁ (position 15 according to Kabat numbering) can be Gly or any amino acid with a reactive group in its side chain, such as cysteine;
X₁₂ (position 17 according to Kabat numbering) can be Ser or any amino acid with a reactive group in its side chain, such as cysteine;
X₁₃ (position 18 according to Kabat numbering) can be Leu or any amino acid with a reactive group in its side chain, such as cysteine;
X₁₄ (position 19 according to Kabat numbering) can be Ser or any amino acid with a reactive group in its side chain, such as cysteine;
X₁₅: (position 21 according to Kabat numbering) can be Ser or any amino acid with a reactive group in its side chain, such as cysteine;
X₁₆: (position 23 according to Kabat numbering) can be Ala or any amino acid with a reactive group in its side chain, such as cysteine;
X₁₇: (position 25 according to Kabat numbering) can be Ser or any amino acid with a reactive group in its side chain, such as cysteine;
X₁₈: (position 26 according to Kabat numbering) can be Gly or any amino acid with a reactive group in its side chain, such as cysteine;
X₁₉: (position 27 according to Kabat numbering) can be Gly or any amino acid with a reactive group in its side chain, such as cysteine;
X₂₀: (position 28 according to Kabat numbering) can be Ser or any amino acid with a reactive group in its side chain, such as cysteine;
X₂₁: (position 30 according to Kabat numbering) can be Ser or any amino acid with a reactive group in its side chain, such as cysteine;
X₂₂: (position 31 according to Kabat numbering) can be Asn or any amino acid with a reactive group in its side chain, such as cysteine;
X₂₃: (position 32 according to Kabat numbering) can be Tyr or any amino acid with a reactive group in its side chain, such as cysteine;
X₂₄: (position 39 according to Kabat numbering) can be Gln or any amino acid with a reactive group in its side chain, such as cysteine;
X₂₅: (position 41 according to Kabat numbering) can be Pro or any amino acid with a reactive group in its side chain, such as cysteine;
X₂₆: (position 42 according to Kabat numbering) can be Gly or any amino acid with a reactive group in its side chain, such as cysteine;
X₂₇: (position 43 according to Kabat numbering) can be Lys or any amino acid with a reactive group in its side chain, such as cysteine;
X₂₈: (position 44 according to Kabat numbering) can be Glu or any amino acid with a reactive group in its side chain, such as cysteine;
X₂₉: (position 45 according to Kabat numbering) can be Arg or any amino acid with a reactive group in its side chain, such as cysteine;
X₃₀: (position 46 according to Kabat numbering) can be Glu or any amino acid with a reactive group in its side chain, such as cysteine;
X₃₁: (position 52a according to Kabat numbering) can be Trp or any amino acid with a reactive group in its side chain, such as cysteine;
X₃₂: (position 53 according to Kabat numbering) can be Arg or any amino acid with a reactive group in its side chain, such as cysteine;
X₃₃: (position 54 according to Kabat numbering) can be Gly or any amino acid with a reactive group in its side chain, such as cysteine;
X₃₄: (position 55 according to Kabat numbering) can be Asp or any amino acid with a reactive group in its side chain, such as cysteine;
X₃₅: (position 56 according to Kabat numbering) can be Ile or any amino acid with a reactive group in its side chain, such as cysteine;
X₃₆: (position 57 according to Kabat numbering) can be Thr or any amino acid with a reactive group in its side chain, such as cysteine;
X₃₇: (position 58 according to Kabat numbering) can be Ile or any amino acid with a reactive group in its side chain, such as cysteine;
X₃₈: (position 59 according to Kabat numbering) can be Gly or any amino acid with a reactive group in its side chain, such as cysteine;
X₃₉: (position 61 according to Kabat numbering) can be Pro or any amino acid with a reactive group in its side chain, such as cysteine;
X₄₀: (position 62 according to Kabat numbering) can be Asn or any amino acid with a reactive group in its side chain, such as cysteine;
X₄₁: (position 64 according to Kabat numbering) can be Glu or any amino acid with a reactive group in its side chain, such as cysteine;
X₄₂: (position 65 according to Kabat numbering) can be Gly or any amino acid with a reactive group in its side chain, such as cysteine;
X₄₃: (position 66 according to Kabat numbering) can be Arg or any amino acid with a reactive group in its side chain, such as cysteine;
X₄₄: (position 68 according to Kabat numbering) can be Thr or any amino acid with a reactive group in its side chain, such as cysteine;
X₄₅: (position 70 according to Kabat numbering) can be Ser or any amino acid with a reactive group in its side chain, such as cysteine;
X₄₆: (position 71 according to Kabat numbering) can be Arg or any amino acid with a reactive group in its side chain, such as cysteine;
X₄₇: (position 72 according to Kabat numbering) can be Asp or any amino acid with a reactive group in its side chain, such as cysteine;
X₄₈: (position 73 according to Kabat numbering) can be Asn or any amino acid with a reactive group in its side chain, such as cysteine;
X₄₉: (position 74 according to Kabat numbering) can be Ala or any amino acid with a reactive group in its side chain, such as cysteine;
X₅₀: (position 75 according to Kabat numbering) can be Lys or any amino acid with a reactive group in its side chain, such as cysteine;
X₅₁: (position 76 according to Kabat numbering) can be Asn or any amino acid with a reactive group in its side chain, such as cysteine;
X₅₂: (position 79 according to Kabat numbering) can be Tyr or any amino acid with a reactive group in its side chain, such as cysteine;
X₅₃: (position 81 according to Kabat numbering) can be Gln or any amino acid with a reactive group in its side chain, such as cysteine;
X₅₄: (position 82a according to Kabat numbering) can be Asn or any amino acid with a reactive group in its side chain, such as cysteine;
X₅₅: (position 82b according to Kabat numbering) can be Ser or any amino acid with a reactive group in its side chain, such as cysteine;
X₅₆: (position 83 according to Kabat numbering) can be Ala or any amino acid with a reactive group in its side chain, such as cysteine;
X₅₇: (position 84 according to Kabat numbering) can be Pro or any amino acid with a reactive group in its side chain, such as cysteine;
X₅₈: (position 85 according to Kabat numbering) can be Asp or any amino acid with a reactive group in its side chain, such as cysteine;
X₅₉: (position 87 according to Kabat numbering) can be Thr or any amino acid with a reactive group in its side chain, such as cysteine;
X₆₀: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val or any other amino acid with a reactive group in its side chain, such as cysteine;
X₆₁: (position 91 according to Kabat numbering) can be Tyr or any amino acid with a reactive group in its side chain, such as cysteine;
X₆₂: (position 96 according to Kabat numbering) can be Thr or any amino acid with a reactive group in its side chain, such as cysteine;
X₆₃: (position 98 according to Kabat numbering) can be Leu or any amino acid with a reactive group in its side chain, such as cysteine;
X₆₄: (position 99 according to Kabat numbering) can be Asn or any amino acid with a reactive group in its side chain, such as cysteine;
X₆₅: (position 100 according to Kabat numbering) can be Pro or any amino acid with a reactive group in its side chain, such as cysteine;
X₆₆: (position 100a according to Kabat numbering) can be Gly or any amino acid with a reactive group in its side chain, such as cysteine;
X₆₇: (position 100d according to Kabat numbering) can be Ile or any amino acid with a reactive group in its side chain, such as cysteine;
X₆₈: (position 100e according to Kabat numbering) can be Tyr or any amino acid with a reactive group in its side chain, such as cysteine;
X₆₉: (position 100f according to Kabat numbering) can be Asp or any amino acid with a reactive group in its side chain, such as cysteine;
X₇₀: (position 100g according to Kabat numbering) can be Trp or any amino acid with a reactive group in its side chain, such as cysteine;
X₇₁: (position 101 according to Kabat numbering) can be Asp or any amino acid with a reactive group in its side chain, such as cysteine;
X₇₂: (position 102 according to Kabat numbering) can be Tyr or any amino acid with a reactive group in its side chain, such as cysteine;
X₇₃: (position 103 according to Kabat numbering) can be Trp or any amino acid with a reactive group in its side chain, such as cysteine;
X₇₄: (position 105 according to Kabat numbering) can be Arg or any amino acid with a reactive group in its side chain, such as cysteine;
X₇₅: (position 106 according to Kabat numbering) can be Gly or any amino acid with a reactive group in its side chain, such as cysteine;
X₇₆: (position 108 according to Kabat numbering) can be Gln, Leu, Arg, Pro, Glu, Lys, Ser, Thr, Met, Ala or His; preferably Gln or Leu, or any other amino acid with a reactive group in its side chain, such as cysteine;
X₇₇: (position 110 according to Kabat numbering) can be Thr or any amino acid with a reactive group in its side chain, such as cysteine;
X₇₈: (position 112 according to Kabat numbering) can be Ser or any amino acid with a reactive group in its side chain, such as cysteine;
X₇₉: (position 113 according to Kabat numbering) can be Ser or any amino acid with a reactive group in its side chain, such as cysteine;
X₈₀: is absent or Gly;
X₈₁: is absent or Gly;
X₈₂: is absent or Cys,
or a sequence which has 80% or more identity with SEQ ID NO.: 186, preferably a sequence which has 85% or more, 90% or more, 95% or more, 97% or more or 99% or more sequence identity with SEQ ID NO.: 186, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one cargo attached to it through the at least one conjugation site or attachment point, does not specifically bind to any non-protein molecule and/or does not specifically bind to any non-human protein to which the ISVD precursor specifically binds.

Preferably, the protein-based carrier building block of the present technology comprises, or alternatively, consists of, SEQ ID NO.: 186 as defined above, wherein one or more of the amino acid residues at positions 11, 37, 44, 45, 47, 83, 84, 103, 104 and 108 according to Kabat numbering are chosen from the Hallmark residues mentioned in Table 3 above.

In a further preferred embodiment, additionally or alternatively, the protein-based carrier building block of the present technology comprises, or alternatively, consists of, SEQ ID NO.: 186 as defined above, wherein SEQ ID NO.: 186 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 186 is preferably Val, and/or the amino acid at position 89 (according to Kabat) in SEQ ID NO.: 186 is preferably Thr or Leu and/or the amino acid at position 110 (according to Kabat) in SEQ ID NO.: 186 is preferably Lys or Gin and/or the amino acid at position 112 (according to Kabat) in SEQ ID NO. 186 is preferably Lys or Gln and/or SEQ ID NO 186 contains a C-terminal extension of 1-5 amino acids chosen from any naturally occurring amino acid.

Hence, the present technology provides a polypeptide which comprises, or alternatively, consists of, SEQ ID NO.: 186 as defined above. Preferably, the polypeptide comprises, or alternatively, consists of, SEQ ID NO.: 186 as defined above, wherein one or more of the amino acid residues at positions 11, 37, 44, 45, 47, 83, 84, 103, 104 and 108 according to Kabat numbering are chosen from the Hallmark residues mentioned in Table 3 above. In a further embodiment, additionally or alternatively, the polypeptide comprises, or alternatively, consists of, SEQ ID NO.: 186 as defined above, wherein SEQ ID NO.: 186 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 186 is preferably Val, and/or the amino acid at position 89 (according to Kabat) in SEQ ID NO.: 186 is preferably Thr or Leu and/or the amino acid at position 110 (according to Kabat) in SEQ ID NO.: 186 is preferably Lys or Gin and/or the amino acid at position 112 (according to Kabat) in SEQ ID NO. 186 is preferably Lys or Gin and/or SEQ ID NO 186 contains a C-terminal extension of 1-5 amino acids chosen from any naturally occurring amino acid.

In one embodiment, the at least one protein-based carrier building block present in the molecule of the present technology comprises, or alternatively, consists of, SEQ ID NO.: 206:

X₁ₐVQLVEX₁GGGZ₁VX₂AGGX₃LX₄IX₅CX₆AX₇X_{7b}GX_{7c}LSX₈YVLGWFRQ
APGX₉X₁₀REFVAAINWRGX₁₁ITIGPPX₁₂VEX₁₃RFX₁₄IX₁₅RX₁₆NX₁₇X₁₈
NTGYLQMNX₁₉LAPX_{19b}DTAZ₂YYCGAGTPLNPX₂₀AYIYX₂₁WSYDYWG
X₂₂GTZ₃VTVX₂₃SX₂₄X₂₅X₂₆

wherein
X₁ₐ (position 1 according to Kabat numbering) can be Glu or any amino acid with a reactive group in its side chain, such as cysteine;
X₁ (position 7 according to Kabat numbering) can be Ser or any amino acid with a reactive group in its side chain, such as cysteine;
Z₁ (position 11 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂ (position 13 according to Kabat numbering) can be Gln or any amino acid with a reactive group in its side chain, such as cysteine;
X₃ (position 17 according to Kabat numbering) can be Ser or any amino acid with a reactive group in its side chain, such as cysteine;
X₄ (position 19 according to Kabat numbering) can be Ser or any amino acid with a reactive group in its side chain, such as cysteine;
X₅: (position 21 according to Kabat numbering) can be Ser or any amino acid with a reactive group in its side chain, such as cysteine;
X₆: (position 23 according to Kabat numbering) can be Ala or any amino acid with a reactive group in its side chain, such as cysteine;
X₇: (position 25 according to Kabat numbering) can be Ser or any amino acid with a reactive group in its side chain, such as cysteine;
X_{7b}: (position 26 according to Kabat numbering) can be Gly or any amino acid with a reactive group in its side chain, such as cysteine;
X_{7c}: (position 28 according to Kabat numbering) can be Ser or any amino acid with a reactive group in its side chain, such as cysteine;
X₈: (position 31 according to Kabat numbering) can be Asn or any amino acid with a reactive group in its side chain, such as cysteine;
X₉: (position 43 according to Kabat numbering) can be Lys or any amino acid with a reactive group in its side chain, such as cysteine;
X₁₀: (position 44 according to Kabat numbering) can be Glu or any amino acid with a reactive group in its side chain, such as cysteine;
X₁₁: (position 55 according to Kabat numbering) can be Asp or any amino acid with a reactive group in its side chain, such as cysteine;
X₁₂: (position 62 according to Kabat numbering) can be Asn or any amino acid with a reactive group in its side chain, such as cysteine;
X₁₃: (position 65 according to Kabat numbering) can be Gly or any amino acid with a reactive group in its side chain, such as cysteine;
X₁₄: (position 68 according to Kabat numbering) can be Thr or any amino acid with a reactive group in its side chain, such as cysteine;
X₁₅: (position 70 according to Kabat numbering) can be Ser or any amino acid with a reactive group in its side chain, such as cysteine;
X₁₆: (position 72 according to Kabat numbering) can be Asp or any amino acid with a reactive group in its side chain, such as cysteine;
X₁₇: (position 74 according to Kabat numbering) can be Ala or any amino acid with a reactive group in its side chain, such as cysteine;
X₁₈: (position 75 according to Kabat numbering) can be Lys or any amino acid with a reactive group in its side chain, such as cysteine;
X₁₉: (position 82b according to Kabat numbering) can be Ser or any amino acid with a reactive group in its side chain, such as cysteine;
X₁₉b: (position 85 according to Kabat numbering) can be Asp or any amino acid with a reactive group in its side chain, such as cysteine;
Z₂: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂₀: (position 100a according to Kabat numbering) can be Gly or any amino acid with a reactive group in its side chain, such as cysteine;
X₂₁: (position 100f according to Kabat numbering) can be Asp or any amino acid with a reactive group in its side chain, such as cysteine;
X₂₂: (position 105 according to Kabat numbering) can be Arg or any amino acid with a reactive group in its side chain, such as cysteine;
Z₃: (position 108 according to Kabat numbering) can be Gln, Leu, Arg, Pro, Glu, Lys, Ser, Thr, Met, Ala or His; preferably Gln or Leu;
X₂₃: (position 112 according to Kabat numbering) can be Ser or any amino acid with a reactive group in its side chain, such as cysteine;
X₂₄: is absent or Gly;
X₂₅: is absent or Gly;
X₂₆: is absent or Cys,
or a sequence which has 80% or more identity with SEQ ID NO.: 206, preferably a sequence which has 85% or more, 90% or more, 95% or more, 97% or more or 99% or more sequence identity with SEQ ID NO.: 206, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one cargo attached to it through the at least one conjugation site or attachment point, does not specifically bind to any non-protein molecule and/or does not specifically bind to any non-human protein to which the ISVD precursor specifically binds.

Preferably, the protein-based carrier building block of the present technology comprises, or alternatively, consists of, SEQ ID NO.: 206, as defined above, wherein one or more of the amino acid residues at positions 11, 37, 44, 45, 47, 83, 84, 103, 104 and 108 according to Kabat numbering are chosen from the Hallmark residues mentioned in Table 3 above.

In a further preferred embodiment, additionally or alternatively, the protein-based carrier building block of the present technology comprises, or alternatively, consists of, SEQ ID NO.: 206 as defined above, wherein SEQ ID NO.: 206 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 206 is preferably Val, and/or the amino acid at position 89 (according to Kabat) in SEQ ID NO.: 206 is preferably Thr or Leu and/or the amino acid at position 110 (according to Kabat) in SEQ ID NO.: 206 is preferably Lys or Gln and/or the amino acid at position 112 (according to Kabat) in SEQ ID NO.: 206 is preferably Lys or Gln and/or SEQ ID NO 206 contains a C-terminal extension of 1-5 amino acids chosen from any naturally occurring amino acid.

Hence, the present technology provides a polypeptide which comprises, or alternatively, consists of, SEQ ID NO.: 206, as defined above. Preferably, the polypeptide comprises, or alternatively, consists of, SEQ ID NO.: 206, as defined above, wherein one or more of the amino acid residues at positions 11, 37, 44, 45, 47, 83, 84, 103, 104 and 108 according to Kabat numbering are chosen from the Hallmark residues mentioned in Table 3 above. In a further embodiment, additionally or alternatively, the polypeptide comprises, or alternatively, consists of, SEQ ID NO.: 206 as defined above, wherein SEQ ID NO.: 206 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 206 is preferably Val, and/or the amino acid at position 89 (according to Kabat) in SEQ ID NO.: 206 is preferably Thr or Leu and/or the amino acid at position 110 (according to Kabat) in SEQ ID NO.: 206 is preferably Lys or Gin and/or the amino acid at position 112 (according to Kabat) in SEQ ID NO.: 206 is preferably Lys or Gln and/or SEQ ID NO.: 206 contains a C-terminal extension of 1-5 amino acids chosen from any naturally occurring amino acid.

In one embodiment, the at least one protein-based carrier building block present in the molecule of the present technology comprises, or alternatively, consists of, SEQ ID NO.: 185:

EVQLVEX₁GGGZ₁VX₂AGGX₃LX₄IX₅CX₆AX₇GGSLSX₈YVLGWFRQAPG
X₉X₁₀REFVAAINWRGX₁₁ITIGPPX₁₂VEX₁₃RFX₁₄₁X₁₅RX₁₆NX₁₇X₁₈NTG
YLQMNX₁₉LAPDDTAZ₂YYCGAGTPLNPX₂₀AYIYX₂₁WSYDYWGX₂₂GTZ₃
VTVX₂₃SX₂₄X₂₅X₂₆

wherein
X₁ (position 7 according to Kabat numbering) can be Ser or any amino acid with a reactive group in its side chain, such as cysteine;
Z₁ (position 11 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂ (position 13 according to Kabat numbering) can be Gln or any amino acid with a reactive group in its side chain, such as cysteine;
X₃ (position 17 according to Kabat numbering) can be Ser or any amino acid with a reactive group in its side chain, such as cysteine;
X₄ (position 19 according to Kabat numbering) can be Ser or any amino acid with a reactive group in its side chain, such as cysteine;
X₅: (position 21 according to Kabat numbering) can be Ser or any amino acid with a reactive group in its side chain, such as cysteine;
X₆: (position 23 according to Kabat numbering) can be Ala or any amino acid with a reactive group in its side chain, such as cysteine;
X₇: (position 25 according to Kabat numbering) can be Ser or any amino acid with a reactive group in its side chain, such as cysteine;
X₈: (position 31 according to Kabat numbering) can be Asn or any amino acid with a reactive group in its side chain, such as cysteine;
X₉: (position 43 according to Kabat numbering) can be Lys or any amino acid with a reactive group in its side chain, such as cysteine;
X₁₀: (position 44 according to Kabat numbering) can be Glu or any amino acid with a reactive group in its side chain, such as cysteine;
X₁₁: (position 55 according to Kabat numbering) can be Asp or any amino acid with a reactive group in its side chain, such as cysteine;
X₁₂: (position 62 according to Kabat numbering) can be Asn or any amino acid with a reactive group in its side chain, such as cysteine;
X₁₃: (position 65 according to Kabat numbering) can be Gly or any amino acid with a reactive group in its side chain, such as cysteine;
X₁₄: (position 68 according to Kabat numbering) can be Thr or any amino acid with a reactive group in its side chain, such as cysteine;
X₁₅: (position 70 according to Kabat numbering) can be Ser or any amino acid with a reactive group in its side chain, such as cysteine;
X₁₆: (position 72 according to Kabat numbering) can be Asp or any amino acid with a reactive group in its side chain, such as cysteine;
X₁₇: (position 74 according to Kabat numbering) can be Ala or any amino acid with a reactive group in its side chain, such as cysteine;
X₁₈: (position 75 according to Kabat numbering) can be Lys or any amino acid with a reactive group in its side chain, such as cysteine;
X₁₉: (position 82b according to Kabat numbering) can be Ser or any amino acid with a reactive group in its side chain, such as cysteine;
Z₂: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂₀: (position 100a according to Kabat numbering) can be Gly or any amino acid with a reactive group in its side chain, such as cysteine;
X₂₁: (position 100f according to Kabat numbering) can be Asp or any amino acid with a reactive group in its side chain, such as cysteine;
X₂₂: (position 105 according to Kabat numbering) can be Arg or any amino acid with a reactive group in its side chain, such as cysteine;
Z₃: (position 108 according to Kabat numbering) can be Gln, Leu, Arg, Pro, Glu, Lys, Ser, Thr, Met, Ala or His; preferably Gln or Leu;
X₂₃: (position 112 according to Kabat numbering) can be Ser or any amino acid with a reactive group in its side chain, such as cysteine;
X₂₄: is absent or Gly;
X₂₅: is absent or Gly;
X₂₆: is absent or Cys,
or a sequence which has 80% or more identity with SEQ ID NO.: 185, preferably a sequence which has 85% or more, 90% or more, 95% or more, 97% or more or 99% or more sequence identity with SEQ ID NO.: 185, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one cargo attached to it through the at least one conjugation site or attachment point, does not specifically bind to any non-protein molecule and/or does not specifically bind to any non-human protein to which the ISVD precursor specifically binds.

Preferably, the protein-based carrier building block of the present technology comprises, or alternatively, consists of, SEQ ID NO.: 185, as defined above, wherein one or more of the amino acid residues at positions 11, 37, 44, 45, 47, 83, 84, 103, 104 and 108 according to Kabat numbering are chosen from the Hallmark residues mentioned in Table 3 above.

In a further preferred embodiment, additionally or alternatively, the protein-based carrier building block of the present technology comprises, or alternatively, consists of, SEQ ID NO.: 185 as defined above, wherein SEQ ID NO.: 185 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 185 is preferably Val, and/or the amino acid at position 89 (according to Kabat) in SEQ ID NO.: 185 preferably Thr or Leu and/or the amino acid at position 110 (according to Kabat) in SEQ ID NO.: 185 is preferably Lys or Gln and/or the amino acid at position 112 (according to Kabat) in SEQ ID NO.: 185 is preferably Lys or Gln and/or SEQ ID NO.: 185 contains a C-terminal extension of 1-5 amino acids chosen from any naturally occurring amino acid.

Hence, the present technology provides a polypeptide which comprises, or alternatively, consists of, SEQ ID NO.: 185, as defined above. Preferably, the polypeptide comprises, or alternatively, consists of, SEQ ID NO.: 185, as defined above, wherein one or more of the amino acid residues at positions 11, 37, 44, 45, 47, 83, 84, 103, 104 and 108 according to Kabat numbering are chosen from the Hallmark residues mentioned in Table 3 above. In a further embodiment, additionally or alternatively, the polypeptide comprises, or alternatively, consists of, SEQ ID NO.: 185 as defined above, wherein SEQ ID NO.: 185 has been further engineered/modified to include mutations which prevent/remove binding by pre-existing antibodies/factors. Examples of such mutations are described, e.g., in WO 2012/175741 and WO 2015/173325. For instance, to prevent/remove binding by pre-existing antibodies/factors, the amino acid at position 11 (according to Kabat) in SEQ ID NO.: 185 is preferably Val, and/or the amino acid at position 89 (according to Kabat) in SEQ ID NO.: 185 is preferably Thr or Leu and/or the amino acid at position 110 (according to Kabat) in SEQ ID NO.: 185 is preferably Lys or Gin and/or the amino acid at position 112 (according to Kabat) in SEQ ID NO. 185 is preferably Lys or Gin and/or SEQ ID NO 18 contains a C-terminal extension of 1-5 amino acids chosen from any naturally occurring amino acid.

5 In one embodiment, the protein-based carrier building block comprises at least one amino acid with a reactive group in its side chain, such as a cysteine, or a lysine, or a tyrosine, or a non-natural amino acid, preferably a cysteine, in at least one of the following solvent-accessible positions, such as three amino acids with a reactive group in its side chain, such as three cysteines, or three lysines, or three tyrosines, or three non-natural amino acids, preferably three cysteines, in the following solvent-accessible positions in SEQ ID NO.: 179 10 according to Kabat numbering:
*43, 100f, 105; or
*43, 75, 100a; or
*21, 68, 100f; or
*7, 44, 55; or
*13, 72, 100a;
*13, 31, 100f; or
*C-terminal Cys (-GGC),
   even more preferably in at least one of the following solvent-accessible positions in SEQ ID NO.: 179 (according to Kabat numbering):
*43, 100f, 105; or
*43, 75, 100a.

Hence, in one embodiment, the protein-based building block of the present technology comprises, or alternatively, consists of, one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent; or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys.

Hence, in one embodiment, the present technology provides a polypeptide which comprises or alternatively consists of, one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys.

For instance, the protein-based carrier building block of the present technology may comprise at least one amino acid with a reactive group in its side chain, such as a cysteine, or a lysine, or a tyrosine, or a non-natural amino acid, preferably a cysteine, in at least one of the following solvent-accessible positions, preferably six amino acids with a reactive group in its side chain, such as six cysteines, or six lysines, or six tyrosines, or six non-natural amino acids, preferably six cysteines, in the following solvent-accessible positions in SEQ ID NO.: 179, or three cysteines and three lysines in the following solvent-accessible positions in SEQ ID NO.: 179, according to Kabat numbering:
*19, 44, 65, 70, 82b, 112; or
*21, 43, 55, 68, 74, 112; or
*19, 23, 31, 70, 82b, 100f;
*13, 25, 43, 65, 72, 100a;
*25, 43, 75, 82b, 100a, 112;
*25, 43, 75, 100a, 105, 112;
*25, 43, 75, 100a, 105, C-terminal Cys (-GGC);
*43, 68, 75, 100a, 105, C-terminal Cys (-GGC);
*25, 43, 75, 100f, 105, C-terminal Cys (-GGC); or
*43, 68, 75, 100f, 105, C-terminal Cys (-GGC).

Hence, in one embodiment, the protein-based building block of the present technology comprises, or alternatively, consists of, one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys.

Hence, in one embodiment, the present technology provides a polypeptide which comprises or alternatively consists of one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) is Gly;
   X₂₁: (position 100f according to Kabat numbering) is Asp, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys.

For instance, the protein-based carrier building block which comprises, or alternatively, consists of, SEQ ID NO.: 179 (or variants thereof with sequence identity of 80% or more, as described above) may comprise at least one amino acid with a reactive group in its side chain, such as a cysteine, or a lysine, or a tyrosine, or a non-natural amino acid, preferably a cysteine, in at least one of the following solvent-accessible positions, preferably nine amino acids with a reactive group in its side chain, such as nine cysteines, or nine lysines, or nine tyrosines, or nine non-natural amino acids, preferably nine cysteines in the following solvent-accessible positions according to Kabat numbering:
*21, 31, 43, 68, 72, 82b, 100a, 100f, 105; or
*7, 13, 19, 23, 44, 55, 62, 70, 74; or
*7, 17, 31, 44, 55, 62, 68, 75, 112.

Hence, in one embodiment, the protein-based building block of the present technology comprises, or alternatively, consists of, one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) can be any amino acid with a reactive group in its side chain, such as a cysteine, or a lysine, or a tyrosine, or a non-natural amino acid, preferably a cysteine;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent.

Hence, in one embodiment, the present technology provides a polypeptide which comprises or alternatively consists of, one of the following sequences:
*SEQ ID NO.: 185, wherein
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
*SEQ ID NO.: 185, wherein
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) can be any amino acid with a reactive group in its side chain, such as a cysteine, or a lysine, or a tyrosine, or a non-natural amino acid, preferably a cysteine;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent.

Additionally, the protein-based carrier building block which comprises, or alternatively, consists of, SEQ ID NO.: 185, 186 and/or 206 (in any of the above-described variants, or variants thereof with sequence identity of 80% or more, as described above) may additionally comprise an extra cysteine and/or an extra tyrosine at one or both ends of the polypeptide defined by SEQ ID NO.: 185, 186 and/or 206 (in any of the above-described variants, or any variant thereof with sequence identity of 80% or more, as described above). If a cysteine is present in the N- and/or C-terminal of the polypeptide defined by SEQ ID NO.: 185, 186 and/or 206 (in any of the above-described variants, or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 185, 186 and/or 206 (in any of the above-described variants, or any variant thereof with sequence identity of 80% or more, as described above), the exposed tyrosine may be preceded/followed by flexible tags, such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086.

In addition, the protein-based carrier building block which comprises, or alternatively, consists of, SEQ ID NO.: 185, 186 and/or 206 (in any of the above-described variants, or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block present in the molecule of the present technology comprises, or alternatively, consists of, one of the sequences of Table 4, or a sequence which has 80% or more identity with a sequence of Table 4, preferably a sequence which has 85% or more, 90% or more, 95% or more, 97% or more or 99% or more sequence identity with a sequence of Table 4, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one cargo attached to it through the at least one conjugation site or attachment point, does not specifically bind to any non-protein molecule and/or does not specifically bind to any non-human protein to which the ISVD precursor specifically binds.

Hence, in one embodiment, the present technology provides a polypeptide which comprises, or alternatively, consists of, one of the sequences of Table 4, or a sequence which has 80% or more identity with a sequence of Table 4, preferably a sequence which has 85% or more, 90% or more, 95% or more, 97% or more or 99% or more sequence identity with a sequence of Table 4

TABLE 4
Examples of ISVD-derived protein-based building blocks.

| ID | SEQ ID NO. | Sequence |
|---|---|---|
| 13001 | 80 | EVQLVESGGGLVQAGGSLSISCAASGGSLSNYVLGWFRQAPG |
| | | CEREFVAAINWRGDITIGPPNVEGRFTISRDNAKNTGYLQMNS |
| | | LAPDDTAVYYCGAGTPLNPGAYIYCWSYDYWGCGTLVTVSS |
| 13002 | 81 | EVQLVESGGGLVQAGGSLSISCAASGGSLSNYVLGWFRQAPG |
| | | CEREFVAAINWRGDITIGPPNVEGRFTISRDNACNTGYLQMNS |
| | | LAPDDTAVYYCGAGTPLNPCAYIYDWSYDYWGRGTLVTVSS |
| 13003 | 82 | EVQLVESGGGLVQAGGSLSICCAASGGSLSNYVLGWFRQAPG |
| | | KEREFVAAINWRGDITIGPPNVEGRFCISRDNAKNTGYLQMNS |
| | | LAPDDTAVYYCGAGTPLNPGAYIYCWSYDYWGRGTLVTVSS |
| 13004 | 83 | EVQLVESGGGLVCAGGSLSISCAASGGSLSNYVLGWFRQAPGK |
| | | EREFVAAINWRGDITIGPPNVEGRFTISRCNAKNTGYLQMNSL |
| | | APDDTAVYYCGAGTPLNPCAYIYDWSYDYWGRGTLVTVSS |
| 13005 | 84 | EVQLVESGGGLVCAGGSLSISCAASGGSLSCYVLGWFRQAPGK |
| | | EREFVAAINWRGDITIGPPNVEGRFTISRDNAKNTGYLQMNSL |
| | | APDDTAVYYCGAGTPLNPGAYIYCWSYDYWGRGTLVTVSS |
| 13006 | 85 | EVQLVECGGGLVQAGGSLSISCAASGGSLSNYVLGWFRQAPG |
| | | KCREFVAAINWRGCITIGPPNVEGRFTISRDNAKNTGYLQMNS |
| | | LAPDDTAVYYCGAGTPLNPGAYIYDWSYDYWGRGTLVTVSS |
| RSVNMP001A04 | 175 | EVQLVESGGGLVQAGGSLSISCAASGGSLSNYVLGWFRQAPG |
| (Q108L)-GGC | | KEREFVAAINWRGDITIGPPNVEGRFTISRDNAKNTGYLQMNS |
| | | LAPDDTAVYYCGAGTPLNPGAYIYDWSYDYWGRGTLVTVSSG |
| | | GC |

In one embodiment, the at least one protein-based carrier building block comprises, or alternatively, consists of, SEQ ID NO.: 80, or a polypeptide which has 80% or more identity with SEQ ID NO.: 80, preferably which has 85% or more, 90% or more, 95% or more, 97% or more or 99% or more sequence identity with SEQ ID NO.: 80, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, as described in detail above. In this embodiment, the amino acids at the solvent-accessible positions 43, 100f and 105 (according to Kabat numbering) are preferably cysteines. Hence, in the building block comprising or consisting of SEQ ID NO.: 80 (or any variant thereof with sequence identity of 80% or more, as described above), positions 43, 100f and 105 (according to Kabat numbering) are solvent-accessible positions, and are preferably occupied by cysteines, which comprise thiol groups, which are the conjugation sites present in the protein building block, preferably does not specifically binds to any non-human protein to which it originally bound, such as bacterial and/or viral proteins, as described in detail above and/or preferably does not specifically binds to any non-protein molecule to which it originally bound, if any, all as described above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. Further, in this embodiment, the amino acid at position 108 (according to Kabat numbering) is preferably leucine. In addition, in this embodiment, the building block comprising or consisting of SEQ ID NO.: 80 (or any variant thereof with sequence identity of 80% or more, as described above) may additionally comprise an extra cysteine and/or an extra tyrosine at one or both ends of the polypeptide defined by SEQ ID NO.: 80 (or any variant thereof with sequence identity of 80% or more, as described above). If a cysteine is present in the N- and/or C-terminal of the polypeptide defined by SEQ ID NO.: 80 (or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 80 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 80 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 81, or a polypeptide which has 80% or more identity with SEQ ID NO.: 81, preferably which has 85% or more, 90% or more, 95% or more, 97% or more or 99% or more sequence identity with SEQ ID NO.: 81, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 43, 75 and 100a (according to Kabat numbering) are preferably cysteines. Hence, in the building block comprising or consisting of SEQ ID NO.: 81 (or any variant thereof with sequence identity of 80% or more, as described above), positions 43, 75 and 100a (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 81 (or any variant thereof with sequence identity of 80% or more, as described above) may additionally comprise an extra cysteine and/or an extra tyrosine at one or both ends of the polypeptide defined by SEQ ID NO.: 81 (or any variant thereof with sequence identity of 80% or more, as described above). If a cysteine is present in the N- and/or C-terminal of the polypeptide defined by SEQ ID NO.: 81 (or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 81 (or any variant thereof with sequence identity of 80% or more, as described above), the tyrosine may be preceded/followed by flexible tags (sequences), such as (GG) or (G₄S₁)₁₋₃GG tags (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 81 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 82, or a polypeptide which has 80% or more identity with SEQ ID NO.: 82, preferably which has 85% or more, 90% or more, 95% or more, 97% or more or 99% or more sequence identity with SEQ ID NO.: 82, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 21, 68 and 100f (according to Kabat numbering) are preferably cysteines. Hence, in the building block comprising or consisting of SEQ ID NO.: 82 (or any variant thereof with sequence identity of 80% or more, as described above), positions 21, 68 and 100f (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 82 (or any variant thereof with sequence identity of 80% or more, as described above) may additionally comprise an extra cysteine and/or an extra tyrosine at one or both ends of the polypeptide defined by SEQ ID NO.: 82 (or any variant thereof with sequence identity of 80% or more, as described above). If a cysteine is present in the N- and/or C-terminal of the polypeptide defined by SEQ ID NO.: 82 (or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 82 (or any variant thereof with sequence identity of 80% or more, as described above), the tyrosine may be preceded/followed by flexible tags (sequences), such as (GG) or (G₄S₁)₁₋₃GG tags (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 82 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 83, or a polypeptide which has 80% or more identity with SEQ ID NO.: 83, preferably which has 85% or more, 90% or more, 95% or more, 97% or more or 99% or more sequence identity with SEQ ID NO.: 83, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 13, 72 and 100a (according to Kabat numbering) are preferably cysteines. Hence, in the building block comprising or consisting of SEQ ID NO.: 83 (or any variant thereof with sequence identity of 80% or more, as described above), positions 13, 72 and 100a (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 83 (or any variant thereof with sequence identity of 80% or more, as described above) may additionally comprise an extra cysteine and/or an extra tyrosine at one or both ends of the polypeptide defined by SEQ ID NO.: 83 (or any variant thereof with sequence identity of 80% or more, as described above). If a cysteine is present in the N- and/or C-terminal of the polypeptide defined by SEQ ID NO.: 83 (or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 83 (or any variant thereof with sequence identity of 80% or more, as described above), the tyrosine may be preceded/followed by flexible tags (sequences), such as (GG) or (G₄S₁)₁₋₃GG tags (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 83 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 84, or a polypeptide which has 80% or more identity with SEQ ID NO.: 84, preferably which has 85% or more, 90% or more, 95% or more, 97% or more or 99% or more sequence identity with SEQ ID NO.: 84, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 13, 31 and 100f (according to Kabat numbering) are preferably cysteines. Hence, in the building block comprising or consisting of SEQ ID NO.: 84 (or any variant thereof with sequence identity of 80% or more, as described above), positions 13, 31 and 100f (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 84 (or any variant thereof with sequence identity of 80% or more, as described above) may additionally comprise an extra cysteine and/or an extra tyrosine at one or both ends of the polypeptide defined by SEQ ID NO.: 84 (or any variant thereof with sequence identity of 80% or more, as described above). If a cysteine is present in the N- and/or C-terminal of the polypeptide defined by SEQ ID NO.: 84 (or any variant thereof with sequence identity of 80% or more, as described above), the cysteine may be preceded/followed by a flexible tag (sequence), such as a (GG) tag (e.g., CGG-or-GGC). If a tyrosine is present in the N- and/or C-terminal of the polypeptide defined by SEQ ID NO.: 84 (or any variant thereof with sequence identity of 80% or more, as described above), the tyrosine may be preceded/followed by flexible tags (sequences), such as (GG) or (G₄S₁)₁₋₃GG tags (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 84 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 85, or a polypeptide which has 80% or more identity with SEQ ID NO.: 85, preferably which has 85% or more, 90% or more, 95% or more, 97% or more or 99% or more sequence identity with SEQ ID NO.: 85, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 7, 44 and 55 (according to Kabat numbering) are preferably cysteines. Hence, in the building block comprising or consisting of SEQ ID NO.: 85 (or any variant thereof with sequence identity of 80% or more, as described above), positions 7, 44 and 55 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 85 (or any variant thereof with sequence identity of 80% or more, as described above) may additionally comprise an extra cysteine and/or an extra tyrosine at one or both ends of the polypeptide defined by SEQ ID NO.: 85 (or any variant thereof with sequence identity of 80% or more, as described above). If a cysteine is present in the N- and/or C-terminal of the polypeptide defined by SEQ ID NO.: 85 (or any variant thereof with sequence identity of 80% or more, as described above), the cysteine may be preceded/followed by a flexible tag (sequence), such as (GG) (e.g., -GGC or CGG-). If a tyrosine is present in the N- and/or C-terminal of the polypeptide defined by SEQ ID NO.: 85 (or any variant thereof with sequence identity of 80% or more, as described above), the tyrosine may be followed and/or preceded by flexible tags (sequences), such as (GG) or (G₄S₁)₁₋₃GG tags (sequences) (e.g., YGG-, -GGY, Y(G₄S₁)₁₋₃GG-, YGG(S₁G₄)₁₋₃-, YGG(G₄S₁)₁₋₃-, or -(G₄S₁)₁₋₃GGY), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 85 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block comprises, or alternatively, consists of, SEQ ID NO.: 175, or a polypeptide which has 80% or more identity with SEQ ID NO.: 175, preferably which has 85% or more, 90% or more, 95% or more, 97% or more or 99% or more sequence identity with SEQ ID NO.: 175, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, a C-terminal cysteine has been engineered in the building-block precursor (-GGC). Hence, in the building block comprising or consisting of SEQ ID NO.: 175 (or any variant thereof with sequence identity of 80% or more, as described above), the C-terminal cysteine comprise a thiol group, which is the conjugation site present in the protein building block, as described above. Further, in this embodiment, the amino acid at position 108 (according to Kabat numbering) is preferably leucine. In addition, in this embodiment, the building block comprising or consisting of SEQ ID NO.: 175 (or any variant thereof with sequence identity of 80% or more, as described above) may additionally comprise an extra cysteine and/or an extra tyrosine at the N-terminal of the polypeptide defined by SEQ ID NO.: 175 (or any variant thereof with sequence identity of 80% or more, as described above). If a cysteine is present in the N-terminal of the polypeptide defined by SEQ ID NO.: 175 (or any variant thereof with sequence identity of 80% or more, as described above), the cysteine may be followed by a flexible tag (sequence), such as a (GG) tag (e.g., CGG-). If a tyrosine is present in the N-terminal of the polypeptide defined by SEQ ID NO.: 175 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 175 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, one of the sequences of Table 5, or a sequence which has 80% or more identity with a sequence of Table 5, preferably a sequence which has 85% or more, 90% or more, 95% or more, 97% or more or 99% or more sequence identity with a sequence of Table 5, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds.

Hence, in another embodiment, the present technology provides a polypeptide which comprises, or alternatively, consists of, one of the sequences of Table 5, or a sequence which has 80% or more identity with a sequence of Table 5, preferably a sequence which has 85% or more, 90% or more, 95% or more, 97% or more or 99% or more sequence identity with a sequence of Table 5.

TABLE 5
Examples of ISVD-derived protein-based building blocks.

| ID | SEQ ID NO. | Sequence |
|---|---|---|
| 16001 | 86 | EVQLVESGGGLVQAGGSLCISCAASGGSLSNYVLGWFRQAPGKCREFVAAI |
| | | NWRGDITIGPPNVECRFTICRDNAKNTGYLQMNCLAPDDTAVYYCGAGTP |
| | | LNPGAYIYDWSYDYWGRGTLVTVCS |
| 16002 | 87 | EVQLVESGGGLVQAGGSLSICCAASGGSLSNYVLGWFRQAPGCEREFVAAI |
| | | NWRGCITIGPPNVEGRFCISRDNCKNTGYLQMNSLAPDDTAVYYCGAGTPL |
| | | NPGAYIYDWSYDYWGRGTLVTVCS |
| 16003 | 88 | EVQLVESGGGLVQAGGSLCISCCASGGSLSCYVLGWFRQAPGKEREFVAAI |
| | | NWRGDITIGPPNVEGRFTICRDNAKNTGYLQMNCLAPDDTAVYYCGAGTP |
| | | LNPGAYIYCWSYDYWGRGTLVTVSS |
| 16004 | 89 | EVQLVESGGGLVCAGGSLSISCAACGGSLSNYVLGWFRQAPGCEREFVAAI |
| | | NWRGDITIGPPNVECRFTISRCNAKNTGYLQMNSLAPDDTAVYYCGAGTPL |
| | | NPCAYIYDWSYDYWGRGTLVTVSS |
| 16005 | 90 | EVQLVESGGGVVQAGGSLSISCAACGGSLSNYVLGWFRQAPGCEREFVAAI |
| | | NWRGDITIGPPNVEGRFTISRDNACNTGYLQMNCLAPDDTALYYCGAGTPL |
| | | NPCAYIYDWSYDYWGRGTLVTVCS |
| 16006 | 91 | EVQLVESGGGVVQAGGSLSISCAACGGSLSNYVLGWFRQAPGCEREFVAAI |
| | | NWRGDITIGPPNVEGRFTISRDNACNTGYLQMNSLAPDDTALYYCGAGTPL |
| | | NPCAYIYDWSYDYWGCGTLVTVCS |
| 16007 | 92 | EVQLVESGGGVVQAGGSLSISCAACGGSLSNYVLGWFRQAPGCEREFVAAI |
| | | NWRGDITIGPPNVEGRFTISRDNACNTGYLQMNSLAPDDTALYYCGAGTPL |
| | | NPCAYIYDWSYDYWGCGTLVTVSSGGC |
| 16008 | 93 | EVQLVESGGGVVQAGGSLSISCAASGGSLSNYVLGWFRQAPGCEREFVAAI |
| | | NWRGDITIGPPNVEGRFCISRDNACNTGYLQMNSLAPDDTALYYCGAGTPL |
| | | NPCAYIYDWSYDYWGCGTLVTVSSGGC |
| 16009 | 94 | EVQLVESGGGVVQAGGSLSISCAACGGSLSNYVLGWFRQAPGCEREFVAAI |
| | | NWRGDITIGPPNVEGRFTISRDNACNTGYLQMNSLAPDDTALYYCGAGTPL |
| | | NPGAYIYCWSYDYWGCGTLVTVSSGGC |
| 16010 | 95 | EVQLVESGGGVVQAGGSLSISCAASGGSLSNYVLGWFRQAPGCEREFVAAI |
| | | NWRGDITIGPPNVEGRFCISRDNACNTGYLQMNSLAPDDTALYYCGAGTPL |
| | | NPGAYIYCWSYDYWGCGTLVTVSSGGC |
| 19001 | 222 | EVQLVESGGGLVQAGGSLSICCAASGGSLSCYVLGWFRQAPGCEREFVAAI |
| | | NWRGDITIGPPNVEGRFCISRCNAKNTGYLQMNCLAPDDTAVYYCGAGTP |
| | | LNPCAYIYCWSYDYWGCGTLVTVSS |
| 19002 | 223 | EVQLVECGGGLVCAGGSLCISCCASGGSLSNYVLGWFRQAPGKCREFVAAI |
| | | NWRGCITIGPPCVEGRFTICRDNCKNTGYLQMNSLAPDDTAVYYCGAGTPL |
| | | NPGAYIYDWSYDYWGRGTLVTVSS |
| 19003 | 224 | EVQLVECGGGLVQAGGCLSISCAASGGSLSCYVLGWFRQAPGKCREFVAAI |
| | | NWRGCITIGPPCVEGRFCISRDNACNTGYLQMNSLAPDDTAVYYCGAGTPL |
| | | NPGAYIYDWSYDYWGRGTLVTVCS |

In one embodiment, the at least one protein-based carrier building block comprises, or alternatively, consists of, SEQ ID NO.: 86, or a polypeptide which has 80% or more identity with SEQ ID NO.: 86, preferably which has 85% or more, 90% or more, 95% or more, 97% or more or 99% or more sequence identity with SEQ ID NO.: 86, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 19, 44, 65, 70, 82b and 112 (according to Kabat numbering) are preferably cysteines. Hence, in the building block comprising or consisting of SEQ ID NO.: 86 (or any variant thereof with sequence identity of 80% or more, as described above), positions 19, 44, 65, 70, 82b and 112 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 86 (or any variant thereof with sequence identity of 80% or more, as described above) may additionally comprise an extra cysteine and/or an extra tyrosine at one or both ends of the polypeptide defined by SEQ ID NO.: 86 (or any variant thereof with sequence identity of 80% or more, as described above). If a cysteine is present in the N- and/or C-terminal of the polypeptide defined by SEQ ID NO.: 86 (or any variant thereof with sequence identity of 80% or more, as described above), the cysteine may be preceded/followed by a flexible tag (sequence), such as (GG) (e.g., -GGC or CGG-). If a tyrosine is present in the N- and/or C-terminal of the polypeptide defined by SEQ ID NO.: 86 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 86 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 87, or a polypeptide which has 80% or more identity with SEQ ID NO.: 87, preferably which has 85% or more, 90% or more, 95% or more, 97% or more or 99% or more sequence identity with SEQ ID NO.: 87, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 21, 43, 55, 68, 74 and 112 (according to Kabat numbering) are preferably cysteines. Hence, in the building block comprising or consisting of SEQ ID NO.: 87 (or any variant thereof with sequence identity of 80% or more, as described above), positions 21, 43, 55, 68, 74 and 112 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 87 (or any variant thereof with sequence identity of 80% or more, as described above) may additionally comprise an extra cysteine and/or an extra tyrosine at one or both ends of the polypeptide defined by SEQ ID NO.: 87 (or any variant thereof with sequence identity of 80% or more, as described above). If a cysteine is present in the N- and/or C-terminal of the polypeptide defined by SEQ ID NO.: 87 (or any variant thereof with sequence identity of 80% or more, as described above), the cysteine may be preceded/followed by a flexible tag (sequence) such as (GG) (e.g., -GGC or CGG-). If a tyrosine is present in the N- and/or C-terminal of the polypeptide defined by SEQ ID NO.: 87 (or any variant thereof with sequence identity of 80% or more, as described above), the tyrosine may be preceded and/or followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 87 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 88, or a polypeptide which has 80% or more identity with SEQ ID NO.: 88, preferably which has 85% or more, 90% or more, 95% or more, 97% or more or 99% or more sequence identity with SEQ ID NO.: 88, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 19, 23, 31, 70, 82b and 100f (according to Kabat numbering) are preferably cysteines. Hence, in the building block comprising or consisting of SEQ ID NO.: 88 (or any variant thereof with sequence identity of 80% or more, as described above), positions 19, 23, 31, 70, 82b and 100f (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 88 (or any variant thereof with sequence identity of 80% or more, as described above) may additionally comprise an extra cysteine and/or an extra tyrosine at one or both ends of the polypeptide defined by SEQ ID NO.: 88 (or any variant thereof with sequence identity of 80% or more, as described above). If a cysteine is present in the N- and/or C-terminal of the polypeptide defined by SEQ ID NO.: 88 (or any variant thereof with sequence identity of 80% or more, as described above), the cysteine may be preceded/followed by a flexible tag (sequence) such as (GG) (e.g., -GGC or CGG-). If a tyrosine is present in the N- and/or C-terminal of the polypeptide defined by SEQ ID NO.: 88 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 88 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 89, or a polypeptide which has 80% or more identity with SEQ ID NO.: 89, preferably which has 85% or more, 90% or more, 95% or more, 97% or more or 99% or more sequence identity with SEQ ID NO.: 89, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 13, 25, 43, 65, 72 and 100a (according to Kabat numbering) are preferably cysteines. Hence, in the building block comprising or consisting of SEQ ID NO.: 89 (or any variant thereof with sequence identity of 80% or more, as described above), positions 13, 25, 43, 65, 72 and 100a (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 89 (or any variant thereof with sequence identity of 80% or more, as described above) may additionally comprise an extra cysteine and/or an extra tyrosine at one or both ends of the polypeptide defined by SEQ ID NO.: 89 (or any variant thereof with sequence identity of 80% or more, as described above). If a cysteine is present in the N- and/or C-terminal of the polypeptide defined by SEQ ID NO.: 89 (or any variant thereof with sequence identity of 80% or more, as described above), the cysteine may be preceded/followed by a flexible tag (sequence) such as (GG) (e.g., -GGC or CGG-). If a tyrosine is present in the N- and/or C-terminal of the polypeptide defined by SEQ ID NO.: 89 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 89 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 90, or a polypeptide which has 80% or more identity with SEQ ID NO.: 90, preferably which has 85% or more, 90% or more, 95% or more, 97% or more or 99% or more sequence identity with SEQ ID NO.: 90, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 25, 43, 75, 82b, 100a and 112 (according to Kabat numbering) are preferably cysteines. Hence, in the building block comprising or consisting of SEQ ID NO.: 90 (or any variant thereof with sequence identity of 80% or more, as described above), positions 25, 43, 75, 82b, 100a and 112 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 90 (or any variant thereof with sequence identity of 80% or more, as described above) may additionally comprise an extra cysteine and/or an extra tyrosine at one or both ends of the polypeptide defined by SEQ ID NO.: 90 (or any variant thereof with sequence identity of 80% or more, as described above). If a cysteine is present in the N- and/or C-terminal of the polypeptide defined by SEQ ID NO.: 90 (or any variant thereof with sequence identity of 80% or more, as described above), the cysteine may be preceded/followed by a flexible tag (sequence) such as (GG) (e.g., -GGC or CGG-). If a tyrosine is present in the N- and/or C-terminal of the polypeptide defined by SEQ ID NO.: 90 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 90 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 91, or a polypeptide which has 80% or more identity with SEQ ID NO.: 91, preferably which has 85% or more, 90% or more, 95% or more, 97% or more or 99% or more sequence identity with SEQ ID NO.: 91, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 25, 43, 75, 100a, 105 and 112 (according to Kabat numbering) are preferably cysteines. Hence, in the building block comprising or consisting of SEQ ID NO.: 91 (or any variant thereof with sequence identity of 80% or more, as described above), positions 25, 43, 75, 100a, 105 and 112 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 91 (or any variant thereof with sequence identity of 80% or more, as described above) may additionally comprise an extra cysteine and/or an extra tyrosine at one or both ends of the polypeptide defined by SEQ ID NO.: 91 (or any variant thereof with sequence identity of 80% or more, as described above). If a cysteine is present in the N- and/or C-terminal of the polypeptide defined by SEQ ID NO.: 91 (or any variant thereof with sequence identity of 80% or more, as described above), the cysteine may be preceded/followed by a flexible tag (sequence) such as (GG) (e.g., -GGC or CGG-). If a tyrosine is present in the N- and/or C-terminal of the polypeptide defined by SEQ ID NO.: 91 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 91 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 92, or a polypeptide which has 80% or more identity with SEQ ID NO.: 92, preferably which has 85% or more, 90% or more, 95% or more, 97% or more or 99% or more sequence identity with SEQ ID NO.: 92, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 25, 43, 75, 100a and 105 (according to Kabat numbering) and the C-terminal amino acid are preferably cysteines. Hence, in the building block comprising or consisting of SEQ ID NO.: 92 (or any variant thereof with sequence identity of 80% or more, as described above), positions 25, 43, 75, 100a and 105 (according to Kabat numbering) and the C-terminal are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 92 (or any variant thereof with sequence identity of 80% or more, as described above) may additionally comprise an extra cysteine and/or an extra tyrosine at the N-terminal of the polypeptide defined by SEQ ID NO.: 92 (or any variant thereof with sequence identity of 80% or more, as described above). If a cysteine is present in the N-terminal of the polypeptide defined by SEQ ID NO.: 92 (or any variant thereof with sequence identity of 80% or more, as described above), the cysteine may be followed by a flexible tag (sequence) such as (GG) (e.g., CGG-). If a tyrosine is present in the N-terminal of the polypeptide defined by SEQ ID NO.: 92 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed-, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 92 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 93, or a polypeptide which has 80% or more identity with SEQ ID NO.: 93, preferably which has 85% or more, 90% or more, 95% or more, 97% or more or 99% or more sequence identity with SEQ ID NO.: 93, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 43, 68, 75, 100a and 105 (according to Kabat numbering) and the C-terminal amino acid are preferably cysteines. Hence, in the building block comprising or consisting of SEQ ID NO.: 93 (or any variant thereof with sequence identity of 80% or more, as described above), positions 43, 68, 75, 100a and 105 (according to Kabat numbering) and the C-terminal are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 93 (or any variant thereof with sequence identity of 80% or more, as described above) may additionally comprise an extra cysteine and/or an extra tyrosine at the N-terminal of the polypeptide defined by SEQ ID NO.: 93 (or any variant thereof with sequence identity of 80% or more, as described above). If a cysteine is present in the N-terminal of the polypeptide defined by SEQ ID NO.: 93 (or any variant thereof with sequence identity of 80% or more, as described above), the cysteine may be followed by a flexible tag (sequence) such as (GG) (e.g., CGG-). If a tyrosine is present in the N-terminal of the polypeptide defined by SEQ ID NO.: 93 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(G₄S₁)₁₋₃-, or YGG(S₁G₄)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 93 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 94, or a polypeptide which has 80% or more identity with SEQ ID NO.: 94, preferably which has 85% or more, 90% or more, 95% or more, 97% or more or 99% or more sequence identity with SEQ ID NO.: 94, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 25, 43, 75, 100f and 105 (according to Kabat numbering) and the C-terminal amino acid are preferably cysteines. Hence, in the building block comprising or consisting of SEQ ID NO.: 94 (or any variant thereof with sequence identity of 80% or more, as described above), positions 25, 43, 75, 100f and 105 (according to Kabat numbering) and the C-terminal are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 94 (or any variant thereof with sequence identity of 80% or more, as described above) may additionally comprise an extra cysteine and/or an extra tyrosine at the N-terminal of the polypeptide defined by SEQ ID NO.: 94 (or any variant thereof with sequence identity of 80% or more, as described above). If a cysteine is present in the N-terminal of the polypeptide defined by SEQ ID NO.: 94 (or any variant thereof with sequence identity of 80% or more, as described above), the cysteine may be followed by a flexible tag (sequence) such as (GG) (e.g., CGG-). If a tyrosine is present in the N-terminal of the polypeptide defined by SEQ ID NO.: 94 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(G₄S₁)₁₋₃-, or YGG(S₁G₄)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 94 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 95, or a polypeptide which has 80% or more identity with SEQ ID NO.: 95, preferably which has 85% or more, 90% or more, 95% or more, 97% or more or 99% or more sequence identity with SEQ ID NO.: 95, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 43, 68, 75, 100f and 105 (according to Kabat numbering) and the C-terminal amino acid are preferably cysteines. Hence, in the building block comprising or consisting of SEQ ID NO.: 95 (or any variant thereof with sequence identity of 80% or more, as described above), positions 43, 68, 75, 100f and 105 (according to Kabat numbering) and the C-terminal are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine, the amino acid at position 11 (according to Kabat numbering) is preferably valine and the amino acid at position 89 (according to Kabat numbering) is preferably leucine. In addition, in this embodiment, the building block comprising or consisting of SEQ ID NO.: 95 (or any variant thereof with sequence identity of 80% or more, as described above) may additionally comprise an extra cysteine and/or an extra tyrosine at the N-terminal of the polypeptide defined by SEQ ID NO.: 95 (or any variant thereof with sequence identity of 80% or more, as described above). If a cysteine is present in the N-terminal of the polypeptide defined by SEQ ID NO.: 95 (or any variant thereof with sequence identity of 80% or more, as described above), the cysteine may be followed by a flexible tag (sequence), such as (GG), e.g., CGG-. If a tyrosine is present in the N-terminal of the polypeptide defined by SEQ ID NO.: 95 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(G₄S₁)₁₋₃-, or YGG(S₁G₄)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 95 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 222, or a polypeptide which has 80% or more identity with SEQ ID NO.: 222, preferably which has 85% or more, 90% or more, 95% or more, 97% or more or 99% or more sequence identity with SEQ ID NO.: 222, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 7, 44 and 55 (according to Kabat numbering) are preferably cysteines. Hence, in the building block comprising or consisting of SEQ ID NO.: 222 (or any variant thereof with sequence identity of 80% or more, as described above), positions 7, 44 and 55 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 222 (or any variant thereof with sequence identity of 80% or more, as described above) may additionally comprise an extra cysteine and/or an extra tyrosine at one or both ends of the polypeptide defined by SEQ ID NO.: 222 (or any variant thereof with sequence identity of 80% or more, as described above). If a cysteine is present in the N- and/or C-terminal of the polypeptide defined by SEQ ID NO.: 222 (or any variant thereof with sequence identity of 80% or more, as described above), the cysteine may be preceded/followed by a flexible tag (sequence), such as (GG) (e.g., -GGC or CGG-). If a tyrosine is present in the N- and/or C-terminal of the polypeptide defined by SEQ ID NO.: 222 (or any variant thereof with sequence identity of 80% or more, as described above), the tyrosine may be followed and/or preceded by flexible tags (sequences), such as (GG) or (G₄S₁)₁₋₃GG tags (sequences) (e.g., YGG-, -GGY, Y(G₄S₁)₁₋₃GG-, YGG(S₁G₄)₁₋₃-, YGG(G₄S₁)₁₋₃-, or -(G₄S₁)₁₋₃GGY), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 222 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 223, or a polypeptide which has 80% or more identity with SEQ ID NO.: 223, preferably which has 85% or more, 90% or more, 95% or more, 97% or more or 99% or more sequence identity with SEQ ID NO.: 223, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, the amino acids at the solvent-accessible positions 7, 44 and 55 (according to Kabat numbering) are preferably cysteines. Hence, in the building block comprising or consisting of SEQ ID NO.: 223 (or any variant thereof with sequence identity of 80% or more, as described above), positions 7, 44 and 55 (according to Kabat numbering) are solvent-accessible positions, and are preferably occupied by cysteines, which comprise thiol groups, which are the conjugation sites present in the protein building block, as described above. Further, in this embodiment, the amino acid at position 108 (according to Kabat numbering) is preferably leucine. In addition, in this embodiment, the building block comprising or consisting of SEQ ID NO.: 223 (or any variant thereof with sequence identity of 80% or more, as described above) may additionally comprise an extra cysteine and/or an extra tyrosine at one or both ends of the polypeptide defined by SEQ ID NO.: 223 (or any variant thereof with sequence identity of 80% or more, as described above). If a cysteine is present in the N- and/or C-terminal of the polypeptide defined by SEQ ID NO.: 223 (or any variant thereof with sequence identity of 80% or more, as described above), the cysteine may be preceded/followed by a flexible tag (sequence), such as (GG) (e.g., -GGC or CGG-). If a tyrosine is present in the N- and/or C-terminal of the polypeptide defined by SEQ ID NO.: 223 (or any variant thereof with sequence identity of 80% or more, as described above), the tyrosine may be followed and/or preceded by flexible tags (sequences), such as (GG) or (G₄S₁)₁₋₃GG tags (sequences) (e.g., YGG-, -GGY, Y(G₄S₁)₁₋₃GG-, YGG(S₁G₄)₁₋₃-, YGG(G₄S₁)₁₋₃-, or -(G₄S₁)₁₋₃GGY), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 223 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block comprises, or alternatively, consists of, SEQ ID NO.: 224, or a polypeptide which has 80% or more identity with SEQ ID NO.: 224, preferably which has 85% or more, 90% or more, 95% or more, 97% or more or 99% or more sequence identity with SEQ ID NO.: 224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, about 7 kDa or about 15 kDa, preferably about 15 or 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cargo, through the at least one conjugation site or attachment point, comprised in the molecule of the present technology, does not specifically bind to any target, such as protein and/or non-protein molecules, including biomolecules, to which the ISVD precursor specifically binds. In this embodiment, a C-terminal cysteine has been engineered in the building-block precursor (-GGC). Hence, in the building block comprising or consisting of SEQ ID NO.: 224 (or any variant thereof with sequence identity of 80% or more, as described above), the C-terminal cysteine comprise a thiol group, which is the conjugation site present in the protein building block, as described above. Further, in this embodiment, the amino acid at position 108 (according to Kabat numbering) is preferably leucine. In addition, in this embodiment, the building block comprising or consisting of SEQ ID NO.: 224 (or any variant thereof with sequence identity of 80% or more, as described above) may additionally comprise an extra cysteine and/or an extra tyrosine at the N-terminal of the polypeptide defined by SEQ ID NO.: 224 (or any variant thereof with sequence identity of 80% or more, as described above). If a cysteine is present in the N-terminal of the polypeptide defined by SEQ ID NO.: 224 (or any variant thereof with sequence identity of 80% or more, as described above), the cysteine may be followed by a flexible tag (sequence), such as a (GG) tag (e.g., CGG-). If a tyrosine is present in the N-terminal of the polypeptide defined by SEQ ID NO.: 224 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 224 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

Without being limiting, advantageous immunoglobulin single variable domains which may be used as starting point (ISDV precursors) for developing the preferred ISVD building block of the present technology are described in WO 2018/099968 and WO 2010/139808. Preferably, the anti-hRSV immunoglobulin single variable domain which may be used as starting point for developing the preferred ISVD building block of the present technology is selected from any of the sequences depicted on Table A-2 on p. 69-70 of WO 2018/099968, incorporated herewith by reference.

An ISVD which may be used as starting point for developing ISVD building blocks according to the present technology is SEQ ID NO.: 5 depicted in on Table A-1, page 388 of WO 2010/139808 (RSV001A04, SEQ ID NO.: 179 in the present description).

### DARPin-Based Building Block

In addition, suitable building blocks in the context of the present technology may be derived from small, globular proteins, as defined above, such as other biologicals, e.g., may be derived from DARPins.

In the context of the present technology, a “DARPin-based building block” refers to a protein-based building block which derives from a DARPin, i.e., which is structurally similar to a DARPin but does not specifically bind to any human protein, preferably does not specifically bind to any target to which the DARPin precursor specifically binds. For instance, the DARPin-based building block has a sequence identity of at least 60%, or 70%, or 80% with a DARPin, e.g., its DARPin precursor. For instance, the DARPin-based building block has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with a DARPin, e.g., its DARPin precursor. For instance, a DARPin-based building block may share the whole amino acid sequence with its DARPin precursor with the exception of at least one, such as one, two, three, four, five, six, seven, eight, nine, ten, fifteen, eighteen, twenty, twenty-five, thirty or more amino acids. In addition, the DARPin-based building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, such as about 6 kDa, or 7 kDa, or 14-16 kDa, does not specifically bind any human protein and preferably does not specifically bind any (non-human) protein or non-protein molecule to which the precursor specifically binds. Preferably, as described above, at least one DARPin-based protein-based building block, preferably when conjugated to at least one cargo, through the at least one conjugation site or attachment point, comprised in the molecule of the present technology, does not specifically bind to any target, such as protein and/or non-protein molecules, including biomolecules, to which the DARPin precursor specifically binds.

DARPins (designed ankyrin repeat proteins) are small, single domain proteins (14-16 kDa) which can be selected to bind any given target protein with high affinity and specificity (from Stumpp M T et al., “DARPins: A new generation of protein therapeutics”, Drug Discovery Today, 2008, 13 (15-16)·695-701). As explained above, the protein-based building block derived from DARPins no longer specifically binds any human protein, i.e., the precursor DARPin as defined in SEQ ID NO.: 187 has been engineered/modified so that it no longer specifically binds any human protein. For instance, DARPin K27 may be modified so that it no longer binds any human protein, as described above, in particular so that it no longer specifically binds human KRAS protein, as described above (or binds it with low specificity/low affinity, as described above). Preferably the protein-based building block derived from DARPins also does not specifically bind to any non-protein molecule (such as DNA, RNA, glycans, lipids (e.g., such as phosphatidylserine (PS)), etc.), preferably it does not specifically bind any human non-protein molecule (such as human DNA, human RNA, human glycans, human lipids (e.g., such as phosphatidylserine (PS)), etc.), preferably it also does not specifically bind to any non-protein molecule (such as DNA, RNA, glycans, lipids (e.g., such as phosphatidylserine (PS)), etc.), to which the building block precursor binds specifically, if any, and preferably it also does not specifically bind to any non-human protein (e.g., a bacterial and/or viral protein) to which the building block precursor binds specifically, if any. Preferably, as described above, at least one DARPin-based protein-based building block, preferably when conjugated to at least one cargo, through the at least one conjugation site or attachment point, comprised in the molecule of the present technology, does not specifically bind to any target, such as protein and/or non-protein molecules, including biomolecules, to which the DARPin precursor specifically binds. Further, preferably, the at least one DARPin-based carrier building block (i) does not specifically bind to any human cell and/or cell type, or binds to a human cell and/or cell type with a K_{D} (K_{D} value) greater than 5×10⁻⁴ mol/litre, preferably as determined by cell-binding assay, (ii) does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein, and/or (iii) does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein.

In one embodiment, the protein-based carrier building block of the present technology is based on the polypeptide as defined in SEQ ID NO.: 187 (DARPin K27 building block precursor):

DLGKKLLEAARAGQDDEVRILMANGADVNAHDTFGFTPLHLAALYGHLE
IVEVLLKNGADVNADDSYGRTPLHLAAMRGHLEIVEVLLKYGADVNAAD
EEGRTPLHLAAKRGHLEIVEVLLKNGADVNAQDKFGKTAFDISIDNGNE
DLAEILQKL

In one embodiment, the protein-based carrier building block of the present technology comprises or, alternatively, consists of, a polypeptide as defined in SEQ ID NO.: 187, or a sequence which has 80% or more identity with SEQ ID NO.: 187, preferably a sequence which has 85% or more, 90% or more, 95% or more, 97% or more or 99% or more sequence identity with SEQ ID NO.: 187, wherein the polypeptide comprises at least one amino acid with a reactive group in its side chain, such as cysteine or lysine, or tyrosine, or a non-natural amino acid, preferably cysteine, in at least one of the following positions in SEQ ID NO.: 187:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 187:
5, 49, 60, 64, 82, 85, 93, 95, 97, 100, 115, 126, 143, 148, 155, more preferably in at least one of the following positions in SEQ ID NO.: 187:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

In another embodiment, the protein-based carrier building block of the present technology comprises or, alternatively, consists of, a polypeptide as defined in SEQ ID NO.: 187, or a sequence which has 80% or more identity with SEQ ID NO.: 187, preferably a sequence which has 85 more, 90% or more, 95% or more, 97% or more or 99% or more sequence identity with SEQ ID NO.: 187, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 187:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 187:
5, 49, 60, 64, 82, 85, 93, 95, 97, 100, 115, 126, 143, 148, 155,
more preferably in more than one of the following positions in SEQ ID NO.: 187:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

Hence, the present technology further provides a polypeptide which comprises or, alternatively, consists of SEQ ID NO.: 187, or a sequence which has 80% or more identity with SEQ ID NO.: 187, preferably a sequence which has 85% or more, 90% or more, 95% or more, 97% or more or 99% or more sequence identity with SEQ ID NO.: 187, wherein the polypeptide comprises at least one amino acid with a reactive group in its side chain, such as cysteine, in at least one of the following positions in SEQ ID NO.: 187:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 187:
5, 49, 60, 64, 82, 85, 93, 95, 97, 100, 115, 126, 143, 148, 155,
more preferably in at least one of the following positions in SEQ ID NO.: 187:
85, 95, 143, 148.

Preferably, the the polypeptide of the present technology comprises or, alternatively, consists of, SEQ ID NO.: 187, or a sequence which has 80% or more identity with SEQ ID NO.: 187, preferably a sequence which has 85% or more, 90% or more, 95% or more, 97% or more or 99% or more sequence identity with SEQ ID NO.: 187, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 187:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 187:
5, 49, 60, 64, 82, 85, 93, 95, 97, 100, 115, 126, 143, 148, 155,
more preferably in more than one of the following positions in SEQ ID NO.: 187:
85, 95, 143, 148.

For instance, the following point mutations can be performed in the polypeptide as defined in SEQ ID NO.: 187, so that it does not longer bind any human protein, in particular so that it does not longer bind the precursor target, e.g., protein KRAS:
R69A, R102A and R111A.

See SEQ ID NO.: 180, which corresponds to SEQ ID NO.: 187 but with the above Arg to Ala mutations at positions 69, 102 and 111:

DLGKKLLEAARAGQDDEVRILMANGADVNAHDTFGFTPLHLAALYGHLE
IVEVLLKNGADVNADDSYGATPLHLAAMRGHLEIVEVLLKYGADVNAAD
EEGATPLHLAAKAGHLEIVEVLLKNGADVNAQDKFGKTAFDISIDNGNE
DLAEILQKL.

In another embodiment, the protein-based carrier building block of the present technology comprises or, alternatively, consists of, a polypeptide as defined in SEQ ID NO.: 180, or a sequence which has 80% or more identity with SEQ ID NO.: 180, preferably a sequence which has 85% or more, 90% or more, 95% or more, 97% or more or 99% or more sequence identity with SEQ ID NO.: 180, wherein the polypeptide comprises at least one amino acid with a reactive group in its side chain, such as cysteine or lysine, or tyrosine, or a non-natural amino acid, preferably cysteine, in at least one of the following positions in SEQ ID NO.: 180:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 180:
5, 49, 60, 64, 82, 85, 93, 95, 97, 100, 115, 126, 143, 148, 155,
more preferably in at least one of the following positions in SEQ ID NO.: 180:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

In another embodiment, the protein-based carrier building block of the present technology comprises or, alternatively, consists of, a polypeptide as defined in SEQ ID NO.: 180, or a sequence which has 80% or more identity with SEQ ID NO.: 180, preferably a sequence which has 85% or more, 90% or more, 95% or more, 97% or more or 99% or more sequence identity with SEQ ID NO.: 180, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 180:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 180:
5, 49, 60, 64, 82, 85, 93, 95, 97, 100, 115, 126, 143, 148, 155,
more preferably in more than one of the following positions in SEQ ID NO.: 180:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

Hence, the present technology further provides a polypeptide which comprises or, alternatively, consists of SEQ ID NO.: 180, or a sequence which has 80% or more identity with SEQ ID NO.: 180, preferably a sequence which has 85% or more, 90% or more, 95% or more, 97% or more or 99% or more sequence identity with SEQ ID NO.: 180, wherein the polypeptide comprises at least one amino acid with a reactive group in its side chain, such as cysteine, in at least one of the following positions in SEQ ID NO.: 180:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 180:
5, 49, 60, 64, 82, 85, 93, 95, 97, 100, 115, 126, 143, 148, 155,
more preferably in at least one of the following positions in SEQ ID NO.: 180:
85, 95, 143, 148.

In one embodiment, the the polypeptide of the present technology comprises or, alternatively, consists of, SEQ ID NO.: 180, or a sequence which has 80% or more identity with SEQ ID NO.: 180, preferably a sequence which has 85% or more, 90% or more, 95% or more, 97% or more or 99% or more sequence identity with SEQ ID NO.: 180, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 180:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 180:
5, 49, 60, 64, 82, 85, 93, 95, 97, 100, 115, 126, 143, 148, 155,
more preferably in more than one of the following positions in SEQ ID NO.: 180:
85, 95, 143, 148.

In another embodiment, the polypeptide as described in SEQ ID NO.: 180 does not have the C-terminal leucine (K27m (without the C-terminal L)), see SEQ ID NO.: 68 and FIG. 2:

DLGKKLLEAARAGQDDEVRILMANGADVNAHDTFGFTPLHLAALYGHLE
IVEVLLKNGADVNADDSYGATPLHLAAMRGHLEIVEVLLKYGADVNAAD
EEGATPLHLAAKAGHLEIVEVLLKNGADVNAQDKFGKTAFDISIDNGNE
DLAEILQK

In one embodiment, the protein-based building block of the present technology does not comprise or consists of a protein with SEQ ID NO.: 180.

Preferably, the protein-based carrier building block of the present technology comprises or, alternatively, consists of, a polypeptide as defined in SEQ ID NO.: 68, or a sequence which has 80% or more identity with SEQ ID NO.: 68, preferably a sequence which has 85% or more, 90% or more, 95% or more, 97% or more or 99% or more sequence identity with SEQ ID NO.: 68, wherein the polypeptide comprises at least one amino acid with a reactive group in its side chain, such as cysteine or lysine, or tyrosine, or a non-natural amino acid, preferably cysteine, in at least one of the following positions in SEQ ID NO.: 68:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 68:
5, 49, 60, 64, 82, 85, 93, 95, 97, 100, 115, 126, 143, 148, 155,
more preferably in at least one of the following positions in SEQ ID NO.: 68:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

Preferably, the protein-based carrier building block of the present technology comprises or, alternatively, consists of, a polypeptide as defined in SEQ ID NO.: 68, or a sequence which has 80% or more identity with SEQ ID NO.: 68, preferably a sequence which has 85% or more, 90% or more, 95% or more, 97% or more or 99% or more sequence identity with SEQ ID NO.: 68, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 68:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 68:
5, 49, 60, 64, 82, 85, 93, 95, 97, 100, 115, 126, 143, 148, 155,
more preferably in more than one of the following positions in SEQ ID NO.: 68:
85, 95, 143, 148,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

Hence, the present technology further provides a polypeptide which comprises or, alternatively, consists of SEQ ID NO.: 68, or a sequence which has 80% or more identity with SEQ ID NO.: 68, preferably a sequence which has 85% or more, 90% or more, 95% or more, 97% or more or 99% or more sequence identity with SEQ ID NO.: 68, wherein the polypeptide comprises at least one amino acid with a reactive group in its side chain, such as cysteine, in at least one of the following positions in SEQ ID NO.: 68:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in at least one of the following positions in SEQ ID NO.: 68:
5, 49, 60, 64, 82, 85, 93, 95, 97, 100, 115, 126, 143, 148, 155,
more preferably in at least one of the following positions in SEQ ID NO.: 68:
85, 95, 143, 148.

Preferably, the the polypeptide of the present technology comprises or, alternatively, consists of, SEQ ID NO.: 68, or a sequence which has 80% or more identity with SEQ ID NO.: 68, preferably a sequence which has 85% or more, 90% or more, 95% or more, 97% or more or 99% or more sequence identity with SEQ ID NO.: 68, wherein the polypeptide comprises more than one, such as two, three, four, five, six, seven, eight, nine, ten, or more, amino acids with a reactive group in its side chain, such as cysteine or lysine, or tyrosine, or a non-natural amino acid, preferably cysteine, in more than one, such as two, three, four, five, six, seven, eight, nine, ten, or more, of the following positions in SEQ ID NO.: 68:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152, 154-155,
preferably in more than one of the following positions in SEQ ID NO.: 68:
5, 49, 60, 64, 82, 85, 93, 95, 97, 100, 115, 126, 143, 148, 155,
more preferably in more than one of the following positions in SEQ ID NO.: 68:
85, 95, 143, 148.

Hence, in one embodiment, the protein-based carrier building block of the present technology comprises or, alternatively, consists of, a polypeptide as defined in SEQ ID NO.: 188:

X₁X₂GX₃X₄LLX₅AAX₆X₇X₈X₉X₁₀X₁₁X₁₂VX₁₃X₁₄LMX₁₅X₁₆X₁₇AX₁₈VX₁₉A
X₂₀X₂₁X₂₂X₂₃GX₂₄TPLHLAAX₂₅X₂₆X₂₇X₂₈X₂₉X₃₀IVX₃₁VLLX₃₂X₃₃X₃₄A
X₃₅VX₃₆AX₃₇DX₃₈X₃₉GATPLHLAAX₄₀X₄₁X₄₂X₄₃X₄₄X₄₅IVX₄₆VLLX₄₇X₄₈
X₄₉AX₅₀VX₅₁AX₅₂DX₅₃X₅₄GATPLHX₅₅AAX₅₆X₅₇X₅₈X₅₉X₆₀X₆₁IVX₆₂X₆₃L
X₆₄X₆₅X₆₆X₆₇AX₆₈X₆₉X₇₀AX₇₁DX₇₂X₇₃X₇₄X₇₅TAX₇₆X₇₇ISX₇₈X₇₉X₈₀X₈₁
X₈₂X₈₃X₈₄LAX₈₅X₈₆LX₈₇X₈₈X₈₉X₉₀,

wherein:
X₁ can be Asp or any amino acid with a reactive group in its side chain, such as cysteine;
X₂ can be Leu or any amino acid with a reactive group in its side chain, such as cysteine;
X₃ can be Lys or any amino acid with a reactive group in its side chain, such as cysteine;
X₄ can be Lys or any amino acid with a reactive group in its side chain, such as cysteine;
X₅ can be Glu or any amino acid with a reactive group in its side chain, such as cysteine;
X₆ can be Arg or any amino acid with a reactive group in its side chain, such as cysteine;
X₇ can be Ala or any amino acid with a reactive group in its side chain, such as cysteine;
X₈ can be Gly or any amino acid with a reactive group in its side chain, such as cysteine;
X₉ can be Gln or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Arg or any amino acid with a reactive group in its side chain, such as cysteine;
X₁₄ can be Ile or any amino acid with a reactive group in its side chain, such as cysteine;
X₁₅ can be Ala or any amino acid with a reactive group in its side chain, such as cysteine;
X₁₆ can be Asn or any amino acid with a reactive group in its side chain, such as cysteine;
X₁₇ can be Gly or any amino acid with a reactive group in its side chain, such as cysteine;
X₁₈ can be Asp or any amino acid with a reactive group in its side chain, such as cysteine;
X₁₉ can be Asn or any amino acid with a reactive group in its side chain, such as cysteine;
X₂₀ can be His or any amino acid with a reactive group in its side chain, such as cysteine;
X₂₁ can be Asp or any amino acid with a reactive group in its side chain, such as cysteine;
X₂₂ can be Thr or any amino acid with a reactive group in its side chain, such as cysteine;
X₂₃ can be Phe or any amino acid with a reactive group in its side chain, such as cysteine;
X₂₄ can be Phe or any amino acid with a reactive group in its side chain, such as cysteine;
X₂₅ can be Leu or any amino acid with a reactive group in its side chain, such as cysteine;
X₂₆ can be Tyr or any amino acid with a reactive group in its side chain, such as cysteine;
X₂₇ can be Gly or any amino acid with a reactive group in its side chain, such as cysteine;
X₂₈ can be His or any amino acid with a reactive group in its side chain, such as cysteine
X₂₉ can be Leu or any amino acid with a reactive group in its side chain, such as cysteine
X₃₀ can be Glu or any amino acid with a reactive group in its side chain, such as cysteine
X₃₁ can be Glu or any amino acid with a reactive group in its side chain, such as cysteine
X₃₂ can be Lys or any amino acid with a reactive group in its side chain, such as cysteine
X₃₃ can be Asn or any amino acid with a reactive group in its side chain, such as cysteine
X₃₄ can be Gly or any amino acid with a reactive group in its side chain, such as cysteine
X₃₅ can be Asp or any amino acid with a reactive group in its side chain, such as cysteine
X₃₆ can be Asn or any amino acid with a reactive group in its side chain, such as cysteine
X₃₇ can be Asp or any amino acid with a reactive group in its side chain, such as cysteine
X₃₈ can be Ser or any amino acid with a reactive group in its side chain, such as cysteine
X₃₉ can be Tyr or any amino acid with a reactive group in its side chain, such as cysteine;
X₄₀ can be Met or any amino acid with a reactive group in its side chain, such as cysteine;
X₄₁ can be Arg or any amino acid with a reactive group in its side chain, such as cysteine;
X₄₂ can be Gly or any amino acid with a reactive group in its side chain, such as cysteine;
X₄₃ can be His or any amino acid with a reactive group in its side chain, such as cysteine;
X₄₄ can be Leu or any amino acid with a reactive group in its side chain, such as cysteine;
X₄₅ can be Glu or any amino acid with a reactive group in its side chain, such as cysteine;
X₄₆ can be Glu or any amino acid with a reactive group in its side chain, such as cysteine;
X₄₇ can be Lys or any amino acid with a reactive group in its side chain, such as cysteine;
X₄₈ can be Tyr or any amino acid with a reactive group in its side chain, such as cysteine;
X₄₉ can be Gly or any amino acid with a reactive group in its side chain, such as cysteine;
X₅₀ can be Asp or any amino acid with a reactive group in its side chain, such as cysteine;
X₅₁ can be Asn or any amino acid with a reactive group in its side chain, such as cysteine;
X₅₂ can be Ala or any amino acid with a reactive group in its side chain, such as cysteine;
X₅₃ can be Glu or any amino acid with a reactive group in its side chain, such as cysteine;
X₅₄ can be Glu or any amino acid with a reactive group in its side chain, such as cysteine;
X₅₅ can be Leu or any amino acid with a reactive group in its side chain, such as cysteine;
X₅₆ can be Lys or any amino acid with a reactive group in its side chain, such as cysteine;
X₅₇ can be Ala or any amino acid with a reactive group in its side chain, such as cysteine;
X₅₈ can be Gly or any amino acid with a reactive group in its side chain, such as cysteine;
X₅₉ can be His or any amino acid with a reactive group in its side chain, such as cysteine;
X₆₀ can be Leu or any amino acid with a reactive group in its side chain, such as cysteine;
X₆₁ can be Glu or any amino acid with a reactive group in its side chain, such as cysteine;
X₆₂ can be Glu or any amino acid with a reactive group in its side chain, such as cysteine;
X₆₃ can be Val or any amino acid with a reactive group in its side chain, such as cysteine;
X₆₄ can be Leu or any amino acid with a reactive group in its side chain, such as cysteine;
X₆₅ can be Lys or any amino acid with a reactive group in its side chain, such as cysteine;
X₆₆ can be Asn or any amino acid with a reactive group in its side chain, such as cysteine;
X₆₇ can be Gly or any amino acid with a reactive group in its side chain, such as cysteine;
X₆₈ can be Asp or any amino acid with a reactive group in its side chain, such as cysteine;
X₆₉ can be Val or any amino acid with a reactive group in its side chain, such as cysteine;
X₇₀ can be Asn or any amino acid with a reactive group in its side chain, such as cysteine;
X₇₁ can be Gln or any amino acid with a reactive group in its side chain, such as cysteine;
X₇₂ can be Lys or any amino acid with a reactive group in its side chain, such as cysteine;
X₇₃ can be Phe or any amino acid with a reactive group in its side chain, such as cysteine;
X₇₄ can be Gly or any amino acid with a reactive group in its side chain, such as cysteine;
X₇₅ can be Lys or any amino acid with a reactive group in its side chain, such as cysteine;
X₇₆ can be Phe or any amino acid with a reactive group in its side chain, such as cysteine;
X₇₇ can be Asp or any amino acid with a reactive group in its side chain, such as cysteine;
X₇₈ can be Ile or any amino acid with a reactive group in its side chain, such as cysteine;
X₇₉ can be Asp or any amino acid with a reactive group in its side chain, such as cysteine;
X₈₀ can be Asn or any amino acid with a reactive group in its side chain, such as cysteine;
X₈₁ can be Gly or any amino acid with a reactive group in its side chain, such as cysteine;
X₈₂ can be Asn or any amino acid with a reactive group in its side chain, such as cysteine;
X₈₃ can be Glu or any amino acid with a reactive group in its side chain, such as cysteine;
X₈₄ can be Asp or any amino acid with a reactive group in its side chain, such as cysteine;
X₈₅ can be Glu or any amino acid with a reactive group in its side chain, such as cysteine;
X₈₆ can be Ile or any amino acid with a reactive group in its side chain, such as cysteine;
X₈₇ can be Gln or any amino acid with a reactive group in its side chain, such as cysteine;
X₈₈ can be Lys or any amino acid with a reactive group in its side chain, such as cysteine;
X₈₉ can be absent or Leu;
X₉₀ can be absent or Cys,
or a sequence which has 80% or more identity with SEQ ID NO.: 188, preferably a sequence which has 85% or more, 90% or more, 95% or more, 97% or more or 99% or more sequence identity with SEQ ID NO.: 188, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above.

Hence, the present technology provides a polypeptide which comprises, or alternatively, consists of, SEQ ID NO.: 188 as defined above, or a sequence which has 80% or more identity with SEQ ID NO.: 188, preferably a sequence which has 85% or more, 90% or more, 95% or more, 97% or more or 99% or more sequence identity with SEQ ID NO.: 188.

In another embodiment, the at least one protein-based carrier building block of the present technology comprises, or alternatively, consists of, SEQ ID NO.: 189:

DLGKX₁LLEAARAGQDDEVRILMANGADVNAHDTFGFTPLHLAALYGHL
X₂IVEVLLKNGAX₃VNAX₄DSYGATPLHLAAMRGHLX₅IVX₆VLLKYGAX₇
VX₈AX₉DEX₁₀GATPLHLAAKAGHLX₁₁IVEVLLKNGAX₁₂VNAQDKFGKTA
FDISIX₁₃NGNEX₁₄LAEILQX₁₅X₁₆X₁₇,

wherein
X₁ can be Lys or any amino acid with a reactive group in its side chain, such as cysteine;
X₂ can be Glu or any amino acid with a reactive group in its side chain, such as cysteine;
X₃ can be Asp or any amino acid with a reactive group in its side chain, such as cysteine;
X₄ can be Asp or any amino acid with a reactive group in its side chain, such as cysteine;
X₅ can be Glu or any amino acid with a reactive group in its side chain, such as cysteine;
X₆ can be Glu or any amino acid with a reactive group in its side chain, such as cysteine;
X₇ can be Asp or any amino acid with a reactive group in its side chain, such as cysteine;
X₈ can be Asn or any amino acid with a reactive group in its side chain, such as cysteine;
X₉ can be Ala or any amino acid with a reactive group in its side chain, such as cysteine;
X₁₀ can be Glu or any amino acid with a reactive group in its side chain, such as cysteine;
X₁₁ can be Glu or any amino acid with a reactive group in its side chain, such as cysteine;
X₁₂ can be Asp or any amino acid with a reactive group in its side chain, such as cysteine;
X₁₃ can be Asp or any amino acid with a reactive group in its side chain, such as cysteine;
X₁₄ can be Asp or any amino acid with a reactive group in its side chain, such as cysteine;
X₁₅ can be Lys or any amino acid with a reactive group in its side chain, such as cysteine;
X₁₆ can be absent or Leu;
X₁₇ can be absent or Cys,
or a sequence which has 80% or more identity with SEQ ID NO.: 189, preferably a sequence which has 85% or more, 90% or more, 95% or more, 97% or more or 99% or more sequence identity with SEQ ID NO.: 189, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above.

Hence, the present technology provides a polypeptide which comprises, or alternatively, consists of, SEQ ID NO.: 189 as defined above, or a sequence which has 80% or more identity with SEQ ID NO.: 189, preferably a sequence which has 85% or more, 90% or more, 95% or more, 97% or more or 99% or more sequence identity with SEQ ID NO.: 189.

In another embodiment, the at least one protein-based carrier building block of the present technology comprises, or alternatively, consists of, SEQ ID NO.: 181:

DLGKKLLEAARAGQDDEVRILMANGADVNAHDTFGFTPLHLAALYGHLE
IVEVLLKNGADVNADDSYGATPLHLAAMRGHLEIVX₁VLLKYGADVX₂AA
DEEGATPLHLAAKAGHLEIVEVLLKNGADVNAQDKFGKTAFDISIX₃NG
NEX₄LAEILQKX₅X₆,

wherein
X₁ can be Glu or any amino acid with a reactive group in its side chain, such as cysteine;
X₂ can be Asn or any amino acid with a reactive group in its side chain, such as cysteine;
X₃ can be Asp or any amino acid with a reactive group in its side chain, such as cysteine;
X₄ can be Asp or any amino acid with a reactive group in its side chain, such as cysteine;
X₅ can be absent or Leu; and
X₆ can be absent or Cys.
or a sequence which has 80% or more identity with SEQ ID NO.: 181, preferably a sequence which has 85% or more, 90% or more, 95% or more, 97% or more or 99% or more sequence identity with SEQ ID NO.: 181, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above.

For instance, the protein-based carrier building block which comprises, or alternatively, consists of, SEQ ID NO.: 181 (or variants thereof with sequence identity of 80% or more, as described above) may comprise at least one amino acid with a reactive group in its side chain, such as a cysteine, or a lysine, or a tyrosine, or a non-natural amino acid, preferably a cysteine, in at least one of the following solvent-accessible positions, such as two amino acids with a reactive group in its side chain, such as two cysteines, or two lysines, or two tyrosines, or two non-natural amino acids, preferably two cysteines in the following solvent-accessible positions (see SEQ ID NO.: 181), and X₅ and X₆ are absent:
X₁ and X₂; or
X₃ and X₄.

For instance, the protein-based carrier building block which comprises, or alternatively, consists of, SEQ ID NO.: 181 (or variants thereof with sequence identity of 80% or more, as described above) may comprise at least one amino acid with a reactive group in its side chain, such as a cysteine, or a lysine, or a tyrosine, or a non-natural amino acid, preferably a cysteine, in at least one of the following solvent-accessible positions, such as four amino acids with a reactive group in its side chain, such as four cysteines, or four lysines, or four tyrosines, or four non-natural amino acids, preferably four cysteines in the following solvent-accessible positions, and X₅ and X₆ are absent:
X₁, X₂, X₃ and X₄, see SEQ ID NO.: 181.

Additionally, the protein-based carrier building block which comprises, or alternatively, consists of, any one of SEQ ID NOs.: 188, 189 or 181 (or variants thereof with sequence identity of 80% or more, as described above) may comprise an extra cysteine and/or an extra tyrosine at one or both ends of the polypeptide defined by SEQ ID NO.: 181 (or any variant thereof with sequence identity of 80% or more, as described above). In a preferred embodiment, the polypeptide defined by any one of SEQ ID NOs.: 188, 189 or 181 comprises one C-terminal cysteine (i.e., X₉₀, X₁₇ and X₆, respectively, are Cys). If a cysteine is present in the N- and/or C-terminal of the polypeptide defined by any one of SEQ ID NOs.: 188, 189 or 181 (or any variant thereof with sequence identity of 80% or more, as described above), the cysteine may be preceded/followed by a flexible tag (sequence), such as a GG tag (e.g., -GGC or CGG-). If a tyrosine is present in the N- and/or C-terminal of the polypeptide defined by any one of SEQ ID NOs.: 188, 189 or 181 (or any variant thereof with sequence identity of 80% or more, as described above), the tyrosine may be preceded/followed by flexible tags (sequences), such as (GG) or (G₄S₁)₁₋₃GG tags (sequences) (e.g., YGG-, -GGY, Y(G₄S₁)₁₋₃GG-, -(G₄S₁)₁₋₃GGY, YGG(S₁G₄)₁₋₃-, or YGG(G₄S₁)₁₋₃-), as described in detail in Alan M. Marmelstein et al., Journal of the American Chemical Society, 2020, 142 (11), 5078-5086. In addition, the protein-based carrier building block which comprises, or alternatively, consists of, any one of SEQ ID NOs.: 188, 189 or 181 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the polypeptide defined by any one of SEQ ID NOs.: 188, 189 or 181 comprises a C-terminal cysteine, preferably wherein the C-terminal cysteine is not preceded by any tag (sequence), see, e.g., SEQ ID NO.: 182, which corresponds to SEQ ID NO.: 181 but comprises a C-terminal Cys (i.e., X₅ in SEQ ID NO.: 181 is absent and X₆ in SEQ IS NO.: 181 is Cys):

DLGKKLLEAARAGQDDEVRILMANGADVNAHDTFGFTPLHLAALYGHLE
IVEVLLKNGADVNADDSYGATPLHLAAMRGHLEIVX₁VLLKYGADVX₂AA
DEEGATPLHLAAKAGHLEIVEVLLKNGADVNAQDKFGKTAFDISIX₃NG
NEX₄LAEILQKC,

wherein
X₁ can be Glu or any amino acid with a reactive group in its side chain, such as cysteine;
X₂ can be Asn or any amino acid with a reactive group in its side chain, such as cysteine;
X₃ can be Asp or any amino acid with a reactive group in its side chain, such as cysteine; and
X₄ can be Asp or any amino acid with a reactive group in its side chain, such as cysteine.

In one embodiment, the at least one protein-based carrier building block present in the molecule of the present technology comprises, or alternatively, consists of, one of the sequences of Table 6, or a sequence which has 80% or more identity with a sequence of Table 6, preferably a sequence which has 85% or more, 90% or more, 95% or more, 97% or more or 99% or more sequence identity with a sequence of Table 6, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above.

Hence, the present technology further provides a polypeptide which comprises or, alternatively, consists of, one of the sequences of Table 6, or a sequence which has 80% or more identity with a sequence of Table 6, preferably a sequence which has 85% or more, 90% or more, 95% or more, 97% or more or 99% or more sequence identity with a sequence of Table 6.

TABLE 6
Examples of protein-based building blocks.

| ID | SEQ ID NO.: | Sequence |
|---|---|---|
| 33001 | 96 | DLGKKLLEAARAGQDDEVRILMANGADVNAHDTFGFTPLHLAALYG |
| | | HLEIVEVLLKNGADVNADDSYGATPLHLAAMRGHLEIVEVLLKYGAD |
| | | VNAADEEGATPLHLAAKAGHLEIVEVLLKNGADVNAQDKFGKTAFDI |
| | | SICNGNECLAEILQKC |
| 33002 | 97 | DLGKKLLEAARAGQDDEVRILMANGADVNAHDTFGFTPLHLAALYG |
| | | HLEIVEVLLKNGADVNADDSYGATPLHLAAMRGHLEIVCVLLKYGAD |
| | | VCAADEEGATPLHLAAKAGHLEIVEVLLKNGADVNAQDKFGKTAFDI |
| | | SIDNGNEDLAEILQKC |
| 35001 | 98 | DLGKKLLEAARAGQDDEVRILMANGADVNAHDTFGFTPLHLAALYG |
| | | HLEIVEVLLKNGADVNADDSYGATPLHLAAMRGHLEIVCVLLKYGAD |
| | | VCAADEEGATPLHLAAKAGHLEIVEVLLKNGADVNAQDKFGKTAFDI |
| | | SICNGNECLAEILQKC |
| K27m | 199 | DLGKKLLEAARAGQDDEVRILMANGADVNAHDTFGFTPLHLAALYGH |
| (156C) | | LEIVEVLLKNGADVNADDSYGATPLHLAAMRGHLEIVEVLLKYGADVN |
| | | AADEEGATPLHLAAKAGHLEIVEVLLKNGADVNAQDKFGKTAFDISID |
| | | NGNEDLAEILQKC |
| K27m | 208 | DLGKKLLEAARAGQDDEVRILMANGADVNAHDTFGFTPLHLAALYGH |
| (156L, | | LEIVEVLLKNGADVNADDSYGATPLHLAAMRGHLEIVEVLLKYGADVN |
| 157C) | | AADEEGATPLHLAAKAGHLEIVEVLLKNGADVNAQDKFGKTAFDISID |
| | | NGNEDLAEILQKLC |

In one embodiment, the at least one protein-based carrier building block comprises, or alternatively, consists of, SEQ ID NO.: 96, or a polypeptide which has 80% or more identity with SEQ ID NO.: 96, preferably which has 85% or more, 90% or more, 95% or more, 97% or more or 99% or more sequence identity with SEQ ID NO.: 96, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically 10 bind to any human protein, in particular does not specifically bind to human KRAS protein, as described in detail above. In this embodiment, the amino acids at the solvent accessible positions 143 and 148 (X₃ and X₄ in SEQ ID NOs.: 181 and 182) and the C-terminal amino acid are preferably cysteines. Hence, in the building block comprising or consisting of SEQ ID NO.: 96 (or any variant thereof with sequence identity of 80% or more, as described above), positions 143 and 148 (X₃ and X₄ in SEQ ID NOs.: 181 and 182) and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 96 (or any variant thereof with sequence identity of 80% or more, as described above) may additionally comprise an extra cysteine and/or an extra tyrosine at the N-terminal of the polypeptide defined by SEQ ID NO.: 96 (or any variant thereof with sequence identity of 80% or more, as described above). If a cysteine is present in the N-terminal of the polypeptide defined by SEQ ID NO.: 96 (or any variant thereof with sequence identity of 80% or more, as described above), the cysteine may be followed by a flexible tag (sequence), such as a GG tag (e.g., CGG-). If a tyrosine is present in the N-terminal of the polypeptide defined by SEQ ID NO.: 96 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG)-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 96 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 97, or a polypeptide which has 80% or more identity with SEQ ID NO.: 97, preferably which has 85% or more, 90% or more, 95% or more, 97% or more or 99% or more sequence identity with SEQ ID NO.: 97, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above. In this embodiment, the amino acids at the solvent accessible positions 85 and 95 (X₁ and X₂ in SEQ ID NOs.: 181 and 182) and the C-terminal amino acid are preferably cysteines. Hence, in the building block comprising or consisting of SEQ ID NO.: 97 (or any variant thereof with sequence identity of 80% or more, as described above), positions 85 and 95 (X₁ and X₂ in SEQ ID NOs.: 181 and 182) and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 97 (or any variant thereof with sequence identity of 80% or more, as described above) may additionally comprise an extra cysteine and/or an extra tyrosine at the N-terminal of the polypeptide defined by SEQ ID NO.: 97 (or any variant thereof with sequence identity of 80% or more, as described above). If a cysteine is present in the N-terminal of the polypeptide defined by SEQ ID NO.: 97 (or any variant thereof with sequence identity of 80% or more, as described above), the cysteine may be followed by a flexible tag (sequence), such as (GG) (e.g., CGG-). If a tyrosine is present in the N-terminal of the polypeptide defined by SEQ ID NO.: 97 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 97 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 98, or a polypeptide which has 80% or more identity with SEQ ID NO.: 98, preferably which has 85% or more, 90% or more, 95% or more, 97% or more or 99% or more sequence identity with SEQ ID NO.: 98, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above. In this embodiment, the amino acids at the solvent accessible positions 85, 95, 143 and 148 (X₁ to X₄ in SEQ ID NOs.: 181 and 182) and the C-terminal amino acid are preferably cysteines. Hence, in the building block comprising or consisting of SEQ ID NO.: 98 (or any variant thereof with sequence identity of 80% or more, as described above), positions 85, 95, 143 and 148 (X₁ to X₄ in SEQ ID NOs.: 181 and 182) and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 98 (or any variant thereof with sequence identity of 80% or more, as described above) may additionally comprise an extra cysteine and/or an extra tyrosine at the N-terminal of the polypeptide defined by SEQ ID NO.: 98 (or any variant thereof with sequence identity of 80% or more, as described above). If a cysteine is present in the N-terminal of the polypeptide defined by SEQ ID NO.: 98 (or any variant thereof with sequence identity of 80% or more, as described above), the cysteine may be followed by a flexible tag, such as (GG) tag (sequence) (e.g., CGG-). If a tyrosine is present in the N-terminal of the polypeptide defined by SEQ ID NO.: 98 (or any variant thereof with sequence identity of 80% or more, as described above), the tyrosine may be followed by flexible tag, such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 98 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block comprises, or alternatively, consists of, SEQ ID NO.: 199, or a polypeptide which has 80% or more identity with SEQ ID NO.: 199, preferably which has 85% or more, 90% or more, 95% or more, 97% or more or 99% or more sequence identity with SEQ ID NO.: 199, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above. In this embodiment, the C-terminal amino acid is a cysteine. Hence, in the building block comprising or consisting of SEQ ID NO.: 199 (or any variant thereof with sequence identity of 80% or more, as described above), the C-terminal cysteine is a solvent-accessible position, which comprises a thiol group, which is a conjugation site present in the protein building block, as described above. In addition, in this embodiment, the building block comprising or consisting of SEQ ID NO.: 199 (or any variant thereof with sequence identity of 80% or more, as described above) may additionally comprise an extra cysteine and/or an extra tyrosine at the N-terminal of the polypeptide defined by SEQ ID NO.: 199 (or any variant thereof with sequence identity of 80% or more, as described above). If a cysteine is present in the N-terminal of the polypeptide defined by SEQ ID NO.: 199 (or any variant thereof with sequence identity of 80% or more, as described above), the cysteine may be followed by a flexible tag (sequence), such as a GG tag (e.g., CGG-). If a tyrosine is present in the N-terminal of the polypeptide defined by SEQ ID NO.: 199 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 199 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block comprises, or alternatively, consists of, SEQ ID NO.: 208, or a polypeptide which has 80% or more identity with SEQ ID NO.: 208, preferably which has 85% or more, 90% or more, 95% or more, 97% or more or 99% or more sequence identity with SEQ ID NO.: 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular does not specifically bind to human KRAS protein, as described in detail above. In this embodiment, the C-terminal amino acid is a cysteine. Hence, in the building block comprising or consisting of SEQ ID NO.: 208 (or any variant thereof with sequence identity of 80% or more, as described above), the C-terminal cysteine is a solvent-accessible position, which comprises a thiol group, which is a conjugation site present in the protein building block, as described above. In addition, in this embodiment, the building block comprising or consisting of SEQ ID NO.: 208 (or any variant thereof with sequence identity of 80% or more, as described above) may additionally comprise an extra cysteine and/or an extra tyrosine at the N-terminal of the polypeptide defined by SEQ ID NO.: 208 (or any variant thereof with sequence identity of 80% or more, as described above). If a cysteine is present in the N-terminal of the polypeptide defined by SEQ ID NO.: 208 (or any variant thereof with sequence identity of 80% or more, as described above), the cysteine may be followed by a flexible tag (sequence), such as a GG tag (e.g., CGG-). If a tyrosine is present in the N-terminal of the polypeptide defined by SEQ ID NO.: 208 (or any variant thereof with sequence identity of 80% or more, as described above), the tyrosine may be followed by flexible tags (sequences), such as (GG) or (G₄S₁)₁₋₃GG tags (sequences) (e.g., YGG-, Y(G₄S₁)₁₋₃GG-, YGG(S₁G₄)₁₋₃-, or YGG(G₄S₁)₁₋₃-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 208 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the DARPin-based building block is not and/or does not comprise the amino acid sequence with SEQ ID NO.: 180. Preferably, the at least one attachment point present in the DARPin-based building block of the present technology is not the C-terminal reactive group, i.e. the —COOH reactive groups present in the C-terminal amino acid of the DARPin-based building block, and/or a primary amine, preferably is not a primary amine present in the side chain of a lysine and/or in the N-terminus. In a preferred embodiment, the at least one attachment point of the DARPin-based building block of the present technology is a thiol group (—SH), preferably present in the side chain of at least one cysteine located at a solvent accessible position in the DARPin-based building block of the present technology.

### Affibody-Based Building Block

Affibody molecules are a class of engineered affinity proteins with proven potential for therapeutic, diagnostic and biotechnological applications. Affibody molecules are small (6.5 kDa) single domain proteins that can be isolated for high affinity and specificity to any given protein target (from FEBS Letters, Volume 584, Issue 12, 18 Jun. 2010, Pages 2670-2680). In the context of the present technology, an “affibody-based building block” refers to a protein-based building block which derives from an affibody, i.e., which is structurally similar to an affibody but does not specifically bind to any human protein, preferably does not specifically bind to any target to which the affibody precursor specifically binds. For instance, the affibody-based building block has a sequence identity of at least 60%, or 70%, or 80% with an affibody, e.g., its affibody precursor. For instance, the affibody-based building block has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with an affibody, e.g., its affibody precursor. For instance, an affibody-based building block may share the whole amino acid sequence with its affibody precursor with the exception of at least one, such as one, two, three, four, five, six, seven, eight, nine, ten, fifteen, eighteen, twenty, twenty-five, thirty or more amino acids. In addition, the affibody-based building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind any human protein and preferably does not specifically bind any (non-human) protein or non-protein molecule to which the precursor specifically binds.

### Affitin-Based Building Block

Affitins are artificial proteins with the ability to selectively bind antigens. They are structurally derived from the DNA-binding protein Sac7d, found in Sulfolobus acidocaldarius. Due to their small size and high solubility, they can be easily produced in large amounts using bacterial expression systems (see, e.g., Kalichuk V. et al., “A novel, smaller scaffold for Affitins: Showcase with binders specific for EpCAM”, Biotechnol Bioeng. 2018; 115 (2): 290-299). In the context of the present technology, an “affitin-based building block” refers to a protein-based building block which derives from an affitin, i.e., which is structurally similar to an affitin but does not specifically bind to any human protein, preferably does not specifically bind to any target to which the affitin precursor specifically binds. For instance, the affitin-based building block has a sequence identity of at least 60%, or 70%, or 80% with an affitin, e.g., its affitin precursor. For instance, the affitin-based building block has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with an affitin, e.g., its affitin precursor. For instance, an affitin-based building block may share the whole amino acid sequence with its affitin precursor with the exception of at least one, such as one, two, three, four, five, six, seven, eight, nine, ten, fifteen, eighteen, twenty, twenty-five, thirty or more amino acids. In addition, the affitin-based building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind any human protein and preferably does not specifically bind any (non-human) protein or non-protein molecule to which the precursor specifically binds.

### Small Globular Human Protein-Based Building Block

The protein-based carrier building block(s) of the present technology may be based on a small globular human protein. In the context of the present technology, a “small globular human protein” refers to a human protein which has a size (molecular mass) of about 2.5 to about 70 kDa, preferably of about 2.5 to about 50 kDa, or of about 2.5 to less than 50 kDa, more preferably of about 2.5 to about 30 kDa, even more preferably of about 2.5 to about 16 kDa, as described herein and which has a globular three-dimensional (3D) structure, as described herein.

Hence, the building block(s) of the present technology may be based on a small globular human protein, such as cyclin-dependent kinase subunit (CKS), e.g., it may be based on cyclin-dependent kinase subunit 1 (CKS1, Gene ID: 983). The binding functionality of the building block precursor (a small globular human protein in this case, such as CKS1) should be eliminated by, e.g., introducing at least one conjugation site in their target binding sites, or, e.g., by mutating residues in or near the binding site: The resulting protein-based building block should not specifically bind any human protein. Furthermore, preferably it also does not specifically bind to any non-protein molecule (such as DNA, RNA, glycans, lipids (e.g., such as phosphatidylserine (PS)), etc.), preferably it also does not specifically bind to any non-protein molecule (such as DNA, RNA, glycans, lipids (e.g., such as phosphatidylserine (PS)), etc.), to which the building block precursor binds specifically, if any, and preferably it also does not specifically bind to any non-human protein (e.g., a bacterial and/or viral protein) to which the building block precursor binds specifically, if any.

Preferably, the small globular human protein on which the protein-based building block may be based, does not comprise any cysteine in its original amino acid sequence, in particular if cysteine-engineering is carried out to create conjugation sites (e.g., if the protein-based carrier building block precursor will be modified by adding cysteines preferably at solvent-accessible positions to generate new conjugation sites or attachment points). In the context of the present technology, a “small globular human protein-based building block” refers to a protein-based building block which derives from a small globular human protein as defined herein, e.g., which is structurally similar to a small globular human protein but does not specifically bind to any human protein, preferably does not specifically bind to any target to which the small globular human protein precursor specifically binds. For instance, the small globular human protein-based building block has a sequence identity of at least 60%, or 70%, or 80% with a small globular human protein, e.g., its small globular human protein precursor. For instance, the small globular human protein-based building block has a sequence identity of at least 85%, such as at least 90%, such as at least 91%, such as at least 92%, such as at least 93%, such as at least 94%, such as at least 95%, such as at least 96%, such as at least 97%, such as at least 98%, such as at least 99%, or more with a small globular human protein, e.g., its small globular human protein precursor. For instance, a small globular human protein-based building block may share the whole amino acid sequence with its small globular human protein precursor with the exception of at least one, such as one, two, three, four, five, six, seven, eight, nine, ten, fifteen, eighteen, twenty, twenty-five, thirty or more amino acids. In addition, the small globular human protein-based building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, does not specifically bind any human protein and preferably does not specifically bind any (non-human) protein or non-protein molecule to which the precursor specifically binds.

In one embodiment, the at least one protein-based carrier building block precursor is a small globular human protein such as CKS (e.g., CKS1). As explained in detail above, the resulting protein-based building block should no longer specifically bind to any human protein. Furthermore, preferably it also does not specifically bind to any non-protein molecule (such as DNA, RNA, glycans, lipids (e.g., such as phosphatidylserine (PS)), etc.), preferably it also does not specifically bind to any non-protein molecule (such as DNA, RNA, glycans, lipids (e.g., such as phosphatidylserine (PS)), etc.), to which the building block precursor binds specifically, if any, and preferably it also does not specifically bind to any non-human protein (e.g., a bacterial and/or viral protein) to which the building block precursor binds specifically, if any.

In one embodiment, the protein-based carrier building block of the present technology is based on the polypeptide as defined in SEQ ID NO.: 190:

SHKQIYYSDKYDDEEFEYRHVMLPKDIAKLVPKTHLMSESEWRNLGVQQ
SQGWVHYMIHEPEPHILLFRRPLPKKPKK.

Preferably, the protein-based carrier building block of the present technology comprises or, alternatively, consists of, a polypeptide as defined in SEQ ID NO.: 190, or a sequence which has 80% or more identity with SEQ ID NO.: 190, preferably a sequence which has 85% or more, 90% or more, 95% or more, 97% or more or 99% or more sequence identity with SEQ ID NO.: 190, wherein the polypeptide comprises at least one amino acid with a reactive group in its side chain, such as cysteine, in at least one of the following positions in SEQ ID NO.: 190:
1-4, 6-7, 9-20, 22, 25-27, 29-30, 32-36, 38-41, 43-44, 46, 48, 50-52, 54, 56-64, 69-78,
preferably in at least one of the following positions in SEQ ID NO.: 190:
9-13, 22, 33, 51, 57 and 78,
or in at least one of the following positions in SEQ ID NO.: 190:
1, 4, 10, 12, 25, 29, 33,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

Preferably, the protein-based carrier building block of the present technology comprises or, alternatively, consists of, a polypeptide as defined in SEQ ID NO.: 190, or a sequence which has 80% or more identity with SEQ ID NO.: 190, preferably a sequence which has 85% or more, 90% or more, 95% or more, 97% or more or 99% or more sequence identity with SEQ ID NO.: 190, wherein the polypeptide comprises more than one, such as two, three, four, five, six, seven, eight, nine, ten, or more, amino acids with a reactive group in its side chain, such as cysteine, in more than one, such as two, three, four, five, six, seven, eight, nine, ten, or more, of the following positions in SEQ ID NO.: 190:
1-4, 6-7, 9-20, 22, 25-27, 29-30, 32-36, 38-41, 43-44, 46, 48, 50-52, 54, 56-64, 69-78,
preferably in at least one of the following positions in SEQ ID NO.: 190:
9-13, 22, 33, 51, 57 and 78,
or in more than one of the following positions in SEQ ID NO.: 190:
1, 4, 10, 12, 25, 29, 33,
provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

Hence, the present technology further provides a polypeptide which comprises or, alternatively, consists of SEQ ID NO.: 190, or a sequence which has 80% or more identity with SEQ ID NO.: 190, preferably a sequence which has 85% or more, 90% or more, 95% or more, 97% or more or 99% or more sequence identity with SEQ ID NO.: 190, wherein the polypeptide comprises at least one amino acid with a reactive group in its side chain, such as cysteine, in at least one of the following positions in SEQ ID NO.: 190:
1-4, 6-7, 9-20, 22, 25-27, 29-30, 32-36, 38-41, 43-44, 46, 48, 50-52, 54, 56-64, 69-78,
preferably in at least one of the following positions in SEQ ID NO.: 190:
9-13, 22, 33, 51, 57 and 78,
or in at least one of the following positions in SEQ ID NO.: 190:
1, 4, 10, 12, 25, 29, 33.

Preferably, the the polypeptide of the present technology comprises or, alternatively, consists of, SEQ ID NO.: 190, or a sequence which has 80% or more identity with SEQ ID NO.: 190, preferably a sequence which has 85% or more, 90% or more, 95% or more, 97% or more or 99% or more sequence identity with SEQ ID NO.: 190, wherein the polypeptide comprises more than one, such as two, three, four, five, six, seven, eight, nine, ten, or more, amino acids with a reactive group in its side chain, such as cysteine, in more than one, such as two, three, four, five, six, seven, eight, nine, ten, or more, of the following positions in SEQ ID NO.: 190:
1-4, 6-7, 9-20, 22, 25-27, 29-30, 32-36, 38-41, 43-44, 46, 48, 50-52, 54, 56-64, 69-78,
preferably in at least one of the following positions in SEQ ID NO.: 190:
9-13, 22, 33, 51, 57 and 78,
or in more than one of the following positions in SEQ ID NO.: 190:
1, 4, 10, 12, 25, 29, 33.

Hence, in one embodiment, the protein-based carrier building block of the present technology comprises or, alternatively, consists of, a polypeptide as defined in SEQ ID NO.: 191:

X₁X₂X₃X₄IX₅X₆SX₇X₈X₉X₁₀X₁₁X₁₂X₁₃X₁₄X₁₅X₁₆X₁₇X₁₈VX₁₉LPX₂₀X₂₁
X₂₂AX₂₃X₂₄VX₂₅X_{23b}X_{24b}X_{25b}X₂₆MX₂₇X₂₈X₂₉X₃₀WX₃₁X₃₂LX₃₃VX₃₄QX₃₅
X₃₆X₃₇WX₃₈HX₃₉X₄₀X₄₁X₄₂X₄₃X₄₄X₄₅X₄₆X₄₇ILLFX₄₈X₄₉X₅₀X₅₁X₅₂X₅₃X₅₄
X₅₅X₅₆X₅₇,

wherein
X₁ can be Ser or any amino acid with a reactive group in its side chain, such as cysteine;
X₂ can be His or any amino acid with a reactive group in its side chain, such as cysteine;
X₃ can be Lys or any amino acid with a reactive group in its side chain, such as cysteine;
X₄ can be Gln or any amino acid with a reactive group in its side chain, such as cysteine;
X₅ can be Tyr or any amino acid with a reactive group in its side chain, such as cysteine;
X₆ can be Tyr or any amino acid with a reactive group in its side chain, such as cysteine;
X₇ can be Asp or any amino acid with a reactive group in its side chain, such as cysteine;
X₈ can be Lys or any amino acid with a reactive group in its side chain, such as cysteine;
X₉ can be Tyr or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Glu or any amino acid with a reactive group in its side chain, such as cysteine;
X₁₄ can be Phe or any amino acid with a reactive group in its side chain, such as cysteine;
X₁₅ can be Glu or any amino acid with a reactive group in its side chain, such as cysteine;
X₁₆ can be Tyr or any amino acid with a reactive group in its side chain, such as cysteine;
X₁₇ can be Arg or any amino acid with a reactive group in its side chain, such as cysteine;
X₁₈ can be His or any amino acid with a reactive group in its side chain, such as cysteine;
X₁₉ can be Met or any amino acid with a reactive group in its side chain, such as cysteine;
X₂₀ can be Lys or any amino acid with a reactive group in its side chain, such as cysteine;
X₂₁ can be Asp or any amino acid with a reactive group in its side chain, such as cysteine;
X₂₂ can be Ile or any amino acid with a reactive group in its side chain, such as cysteine;
X₂₃ can be Lys or any amino acid with a reactive group in its side chain, such as cysteine;
X₂₄ can be Leu or any amino acid with a reactive group in its side chain, such as cysteine;
X₂₅ can be Pro or any amino acid with a reactive group in its side chain, such as cysteine;
X₂₃b can be Lys or any amino acid with a reactive group in its side chain, such as cysteine;
X₂₄b can be Thr or any amino acid with a reactive group in its side chain, such as cysteine;
X₂₅b can be His or any amino acid with a reactive group in its side chain, such as cysteine;
X₂₆ can be Leu or any amino acid with a reactive group in its side chain, such as cysteine;
X₂₇ can be Ser or any amino acid with a reactive group in its side chain, such as cysteine;
X₂₈ can be Glu or any amino acid with a reactive group in its side chain, such as cysteine;
X₂₉ can be Ser or any amino acid with a reactive group in its side chain, such as cysteine;
X₃₀ can be Glu or any amino acid with a reactive group in its side chain, such as cysteine;
X₃₁ can be Arg or any amino acid with a reactive group in its side chain, such as cysteine;
X₃₂ can be Asn or any amino acid with a reactive group in its side chain, such as cysteine;
X₃₃ can be Gly or any amino acid with a reactive group in its side chain, such as cysteine;
X₃₄ can be Gln or any amino acid with a reactive group in its side chain, such as cysteine;
X₃₅ can be Ser or any amino acid with a reactive group in its side chain, such as cysteine;
X₃₆ can be Gln or any amino acid with a reactive group in its side chain, such as cysteine;
X₃₇ can be Gly or any amino acid with a reactive group in its side chain, such as cysteine;
X₃₈ can be Val or any amino acid with a reactive group in its side chain, such as cysteine;
X₃₉ can be Tyr or any amino acid with a reactive group in its side chain, such as cysteine;
X₄₀ can be Met or any amino acid with a reactive group in its side chain, such as cysteine;
X₄₁ can be Ile or any amino acid with a reactive group in its side chain, such as cysteine;
X₄₂ can be His or any amino acid with a reactive group in its side chain, such as cysteine;
X₄₃ can be Glu or any amino acid with a reactive group in its side chain, such as cysteine;
X₄₄ can be Pro or any amino acid with a reactive group in its side chain, such as cysteine;
X₄₅ can be Glu or any amino acid with a reactive group in its side chain, such as cysteine;
X₄₆ can be Pro or any amino acid with a reactive group in its side chain, such as cysteine;
X₄₇ can be His or any amino acid with a reactive group in its side chain, such as cysteine;
X₄₈ can be Arg or any amino acid with a reactive group in its side chain, such as cysteine;
X₄₉ can be Arg or any amino acid with a reactive group in its side chain, such as cysteine;
X₅₀ can be Pro or any amino acid with a reactive group in its side chain, such as cysteine;
X₅₁ can be Leu or any amino acid with a reactive group in its side chain, such as cysteine;
X₅₂ can be Pro or any amino acid with a reactive group in its side chain, such as cysteine;
X₅₃ can be Lys or any amino acid with a reactive group in its side chain, such as cysteine;
X₅₄ can be Lys or any amino acid with a reactive group in its side chain, such as cysteine;
X₅₅ can be Pro or any amino acid with a reactive group in its side chain, such as cysteine;
X₅₆ can be Lys or any amino acid with a reactive group in its side chain, such as cysteine;
X₅₇ can be Lys or any amino acid with a reactive group in its side chain, such as cysteine,
or a sequence which has 80% or more identity with SEQ ID NO.: 191, preferably a sequence which has 85% or more, 90% or more, 95% or more, 97% or more or 99% or more sequence identity with SEQ ID NO.: 191, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

Hence, the present technology provides a polypeptide which comprises, or alternatively, consists of, SEQ ID NO.: 191 as defined above, or a sequence which has 80% or more identity with SEQ ID NO.: 191, preferably a sequence which has 85% or more, 90% or more, 95% or more, 97% or more or 99% or more sequence identity with SEQ ID NO.: 191.

In another embodiment, the at least one protein-based carrier building block of the present technology comprises, or alternatively, consists of, SEQ ID NO.: 205:

SHKQIYYSX₁X₂X₃X₄X₅EEFEYRHVX₆LPKDIAKLVPX₇THLMSESEWRN
LGVQQSX₈GWVHYX₉IHEPEPHILLFRRPLPKKPKX₁₀,

wherein
X₁ can be Asp or any amino acid with a reactive group in its side chain, such as cysteine;
X₂ can be Lys or any amino acid with a reactive group in its side chain, such as cysteine;
X₃ can be Tyr or any amino acid with a reactive group in its side chain, such as cysteine;
X₄ can be Asp or any amino acid with a reactive group in its side chain, such as cysteine;
X₅ can be Asp or any amino acid with a reactive group in its side chain, such as cysteine;
X₆ can be Met or any amino acid with a reactive group in its side chain, such as cysteine;
X₇ can be Lys or any amino acid with a reactive group in its side chain, such as cysteine;
X₈ can be Gln or any amino acid with a reactive group in its side chain, such as cysteine;
X₉ can be Met or any amino acid with a reactive group in its side chain, such as cysteine;
X₁₀ can be Lys or any amino acid with a reactive group in its side chain, such as cysteine,
or a sequence which has 80% or more identity with SEQ ID NO.: 205, preferably a sequence which has 85% or more, 90% or more, 95% or more, 97% or more or 99% or more sequence identity with SEQ ID NO.: 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

Hence, the present technology provides a polypeptide which comprises, or alternatively, consists of, SEQ ID NO.: 205 as defined above, or a sequence which has 80% or more identity with SEQ ID NO.: 205, preferably a sequence which has 85% or more, 90% or more, 95% or more, 97% or more or 99% or more sequence identity with SEQ ID NO.: 205.

In another embodiment, the at least one protein-based carrier building block of the present technology comprises, or alternatively, consists of, SEQ ID NO.: 192:

X₁HKX₂IYYSDX₃YX₄DEEFEYRHVMLPX₅DIAX₆LVPX₇THLMSESEWRN
LGVQQSQGWVHYMIHEPEPHILLFRRPLPKKPKK,

wherein
X₁ can be Ser or any amino acid with a reactive group in its side chain, such as cysteine;
X₂ can be Gln or any amino acid with a reactive group in its side chain, such as cysteine;
X₃ can be Lys or any amino acid with a reactive group in its side chain, such as cysteine;
X₄ can be Asp or any amino acid with a reactive group in its side chain, such as cysteine;
X₅ can be Lys or any amino acid with a reactive group in its side chain, such as cysteine;
X₆ can be Lys or any amino acid with a reactive group in its side chain, such as cysteine;
X₇ can be Lys or any amino acid with a reactive group in its side chain, such as cysteine,
or a sequence which has 80% or more identity with SEQ ID NO.: 192, preferably a sequence which has 85% or more, 90% or more, 95% or more, 97% or more or 99% or more sequence identity with SEQ ID NO.: 192, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

Hence, the present technology provides a polypeptide which comprises, or alternatively, consists of, SEQ ID NO.: 192 as defined above, or a sequence which has 80% or more identity with SEQ ID NO.: 192, preferably a sequence which has 85% or more, 90% or more, 95% or more, 97% or more or 99% or more sequence identity with SEQ ID NO.: 192.

Additionally, the protein-based carrier building block which comprises, or alternatively, consists of, any one of SEQ ID NOs.: 191, 192 or 205 (or variants thereof with sequence identity of 80% or more, as described above) may additionally comprise an extra cysteine and/or an extra tyrosine at one or both ends of the polypeptide defined by any one of SEQ ID NOs.: 191, 192 or 205 (or any variant thereof with sequence identity of 80% or more, as described above). If a cysteine is present in the N- and/or C-terminal of the polypeptide defined by any one of SEQ ID NOs.: 191, 192 or 205 (or any variant thereof with sequence identity of 80% or more, as described above), the cysteine may be preceded/followed by a flexible tag (sequence), such as a (GG) sequence (e.g., -GGC or CGG-). If a tyrosine is present in the N- and/or C-terminal of the polypeptide defined by any one of SEQ ID NOs.: 191, 192 or 205 (or any variant thereof with sequence identity of 80% or more, as described above), the tyrosine may be preceded by flexible tags (sequences), such as (GG) or (G₄S₁)₁₋₃GG tags (sequences) (e.g., YGG-, -GGY, Y(G₄S₁)₁₋₃GG-, YGG(G₄S₁)₁₋₃-, YGG(S₁G₄)₁₋₃- or -(G₄S₁)₁₋₃GGY), preferably -GGY, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, any one of SEQ ID NOs.: 191, 192 or 205 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In one embodiment, the at least one protein-based carrier building block of the present technology comprises, or alternatively, consists of, one of the sequences of Table 7, or a sequence which has 80% or more identity with a sequence of Table 7, preferably a sequence which has 85% or more, 90% or more, 95% or more, 97% or more or 99% or more sequence identity with a sequence of Table 7, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above.

In another embodiment, the present technology also provides a polypeptide which comprises, or alternatively, consists of, one of the sequences of Table 7, or a sequence which has 80% or more identity with a sequence of Table 7, preferably a sequence which has 85% or more, 90% or more, 95% or more, 97% or more or 99% or more sequence identity with a sequence of Table 7.

TABLE 7
Examples of protein-based building blocks.

| ID | SEQ ID NO. | Sequence |
|---|---|---|
| 23001 | 99 | SHKQIYYSDCYDCEEFEYRHVMLPKDIAKLVPKTHLMSESEWRNLGVQQSQ |
| | | GWVHYMIHEPEPHILLFRRPLPKKPKC |
| 23002 | 100 | SHKQIYYSDCYCDEEFEYRHVCLPKDIAKLVPKTHLMSESEWRNLGVQQSCG |
| | | WVHYMIHEPEPHILLFRRPLPKKPKK |
| 23003 | 101 | SHKQIYYSDKCDDEEFEYRHVMLPKDIAKLVPKTHLMSESEWRNLGVQQSC |
| | | GWVHYMIHEPEPHILLFRRPLPKKPKC |
| 23004 | 102 | SHKQIYYSDKYDCEEFEYRHVMLPKDIAKLVPCTHLMSESEWRNLGVQQSQ |
| | | GWVHYMIHEPEPHILLFRRPLPKKPKC |
| 23005 | 103 | SHKQIYYSDKCDDEEFEYRHVMLPKDIAKLVPCTHLMSESEWRNLGVQQSC |
| | | GWVHYMIHEPEPHILLFRRPLPKKPKC |
| 26001 | 104 | SHKQIYYSCKYDCEEFEYRHVCLPKDIAKLVPCTHLMSESEWRNLGVQQSCG |
| | | WVHYMIHEPEPHILLFRRPLPKKPKC |
| 26002 | 105 | SHKQIYYSCKYDCEEFEYRHVMLPKDIAKLVPCTHLMSESEWRNLGVQQSC |
| | | GWVHYCIHEPEPHILLFRRPLC |

In one embodiment, the at least one protein-based carrier building block comprises, or alternatively, consists of, SEQ ID NO.: 99, or a polypeptide which has 80% or more identity with SEQ ID NO.: 99, preferably which has 85% or more, 90% or more, 95% or more, 97% or more or 99% or more sequence identity with SEQ ID NO.: 99, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 10 and 13 and the C-terminal amino acid are preferably cysteines. Hence, in the building block comprising or consisting of SEQ ID NO.: 99 (or any variant thereof with sequence identity of 80% or more, as described above), positions 10 and 13 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 99 (or any variant thereof with sequence identity of 80% or more, as described above) may additionally comprise an extra cysteine and/or an extra tyrosine at the N-terminal end of the polypeptide defined by SEQ ID NO.: 99 (or any variant thereof with sequence identity of 80% or more, as described above). If a cysteine is present at the N-terminal of the polypeptide defined by SEQ ID NO.: 99 (or any variant thereof with sequence identity of 80% or more, as described above), the cysteine may be followed by a flexible tag, such as (GG) tag (sequence) (e.g., CGG-). If a tyrosine is present at the N-terminal of the polypeptide defined by SEQ ID NO.: 99 (or any variant thereof with sequence identity of 80% or more, as described above), the tyrosine may be followed by flexible tags (sequences), such as (GG) or (G₄S₁)₁₋₃GG tags (sequences) (YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 99 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 100, or a polypeptide which has 80% or more identity with SEQ ID NO.: 100, preferably which has 85% or more, 90% or more, 95% or more, 97% or more or 99% or more sequence identity with SEQ ID NO.: 100, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 10, 12, 22 and 51 are preferably cysteines. Hence, in the building block comprising or consisting of SEQ ID NO.: 100 (or any variant thereof with sequence identity of 80% or more, as described above), positions 10, 12, 22 and 51 are solvent-accessible positions, and are preferably occupied by cysteines, which comprise thiol groups, which are the conjugation sites present in the protein building block, as described above. In addition, in this embodiment, the building block comprising or consisting of SEQ ID NO.: 100 (or any variant thereof with sequence identity of 80% or more, as described above) may additionally comprise an extra cysteine and/or an extra tyrosine at one or both ends of the polypeptide defined by SEQ ID NO.: 100 (or any variant thereof with sequence identity of 80% or more, as described above). If a cysteine is present in the N- and/or C-terminal of the polypeptide defined by SEQ ID NO.: 100 (or any variant thereof with sequence identity of 80% or more, as described above), the cysteine may be preceded/followed by a flexible tag (sequence, such as (GG) (e.g., -GGC or CGG-). If a tyrosine is present in the N- and/or C-terminal of the polypeptide defined by SEQ ID NO.: 100 (or any variant thereof with sequence identity of 80% or more, as described above), the tyrosine may be preceded/followed by flexible tags (sequences) such as (GG) or (G₄S₁)₁₋₃GG tags (sequences) (e.g., YGG-, -GGY, -(G₄S₁)₁₋₃GGY, YGG(G₄S₁)₁₋₃-, YGG(S₁G₄)₁₋₃- or Y(G₄S₁)₁₋₃GG-), preferably -GGY or YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, SEQ ID NO.: 100 (or variants thereof with sequence identity of 80% or more, as described above) may additionally comprise a sortase-recognition motif (LPXTG) at the C-terminal end, and/or a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 101, or a polypeptide which has 80% or more identity with SEQ ID NO.: 101, preferably which has 85% or more, 90% or more, 95% or more, 97% or more or 99% or more sequence identity with SEQ ID NO.: 101, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 11 and 51 and the C-terminal amino acid are preferably cysteines. Hence, in the building block comprising or consisting of SEQ ID NO.: 101 (or any variant thereof with sequence identity of 80% or more, as described above), positions 11 and 51 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 101 (or any variant thereof with sequence identity of 80% or more, as described above) may additionally comprise an extra cysteine and/or an extra tyrosine at the N-terminal end of the polypeptide defined by SEQ ID NO.: 101 (or any variant thereof with sequence identity of 80% or more, as described above). If a cysteine is present at the N-terminal of the polypeptide defined by SEQ ID NO.: 101 (or any variant thereof with sequence identity of 80% or more, as described above), the cysteine may be followed by a flexible tag (sequence), such as (GG) (e.g., CGG-). If a tyrosine is present at the N-terminal of the polypeptide defined by SEQ ID NO.: 101 (or any variant thereof with sequence identity of 80% or more, as described above), the tyrosine may be followed by flexible tags, such as (GG) or (G₄S₁)₁₋₃GG tags (sequences) (e.g., YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 101 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 102, or a polypeptide which has 80% or more identity with SEQ ID NO.: 102, preferably which has 85% or more, 90% or more, 95% or more, 97% or more or 99% or more sequence identity with SEQ ID NO.: 102, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 13 and 33 and the C-terminal amino acid are preferably cysteines. Hence, in the building block comprising or consisting of SEQ ID NO.: 102 (or any variant thereof with sequence identity of 80% or more, as described above), positions 13 and 33 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 102 (or any variant thereof with sequence identity of 80% or more, as described above) may additionally comprise an extra cysteine and/or an extra tyrosine at the N-terminal ends of the polypeptide defined by SEQ ID NO.: 102 (or any variant thereof with sequence identity of 80% or more, as described above). If a cysteine is present at the N-terminal of the polypeptide defined by SEQ ID NO.: 102 (or any variant thereof with sequence identity of 80% or more, as described above), the cysteine may be followed by a flexible tag (sequence), such as (GG) (e.g., CGG-). If a tyrosine is present in the N-terminal of the polypeptide defined by SEQ ID NO.: 102 (or any variant thereof with sequence identity of 80% or more, as described above), the tyrosine may be followed by flexible tags (sequences), such as (GG) or (G₄S₁)₁₋₃GG tags (sequences) (e.g., YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 102 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 103, or a polypeptide which has 80% or more identity with SEQ ID NO.: 103, preferably which has 85% or more, 90% or more, 95% or more, 97% or more or 99% or more sequence identity with SEQ ID NO.: 103, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 11, 33, and 51 and the C-terminal amino acid are preferably cysteines. Hence, in the building block comprising or consisting of SEQ ID NO.: 103 (or any variant thereof with sequence identity of 80% or more, as described above), positions 11, 33, and 51 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 103 (or any variant thereof with sequence identity of 80% or more, as described above) may additionally comprise an extra cysteine and/or an extra tyrosine at the N-terminal end of the polypeptide defined by SEQ ID NO.: 103 (or any variant thereof with sequence identity of 80% or more, as described above). If a cysteine is present at the N-terminal of the polypeptide defined by SEQ ID NO.: 103 (or any variant thereof with sequence identity of 80% or more, as described above), the cysteine may be followed by a flexible tag (sequence), such as (GG) (e.g., CGG-). If a tyrosine is present in the N-terminal of the polypeptide defined by SEQ ID NO.: 103 (or any variant thereof with sequence identity of 80% or more, as described above), the tyrosine may be followed by flexible tags (sequences), such as (GG) or (G₄S₁)₁₋₃GG tags (sequences) (e.g., YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 103 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 104, or a polypeptide which has 80% or more identity with SEQ ID NO.: 104, preferably which has 85% or more, 90% or more, 95% or more, 97% or more or 99% or more sequence identity with SEQ ID NO.: 104, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 9, 13, 22, 33 and 51 and the C-terminal amino acid are preferably cysteines. Hence, in the building block comprising or consisting of SEQ ID NO.: 104 (or any variant thereof with sequence identity of 80% or more, as described above), positions 9, 13, 22, 33 and 51 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 104 (or any variant thereof with sequence identity of 80% or more, as described above) may additionally comprise an extra cysteine and/or an extra tyrosine at the N-terminal end of the polypeptide defined by SEQ ID NO.: 104 (or any variant thereof with sequence identity of 80% or more, as described above). If a cysteine is present in the N-terminal of the polypeptide defined by SEQ ID NO.: 104 (or any variant thereof with sequence identity of 80% or more, as described above), the cysteine may be followed by a flexible tag (sequence), such as (GG) (e.g., CGG-). If a tyrosine is present in the N-terminal of the polypeptide defined by SEQ ID NO.: 104 (or any variant thereof with sequence identity of 80% or more, as described above), the tyrosine may be followed by flexible tags (sequences), such as (GG) or (G₄S₁)₁₋₃GG tags (sequences) (e.g., YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 104 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

In another embodiment, the at least one protein-based carrier building block comprises, or alternatively, consists of, SEQ ID NO.: 105, or a polypeptide which has 80% or more identity with SEQ ID NO.: 105, preferably which has 85% or more, 90% or more, 95% or more, 97% or more or 99% or more sequence identity with SEQ ID NO.: 105, provided that the building block has a globular 3D structure, is soluble, has a size (molecular mass) of about 2.5 to about 70 kDa, such about 2.5 to about 50 kDa, or of as about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, as described in detail above. In this embodiment, the amino acids at the solvent-accessible positions 9, 13, 33, 51 and 57 and the C-terminal amino acid are preferably cysteines. Hence, in the building block comprising or consisting of SEQ ID NO.: 105 (or any variant thereof with sequence identity of 80% or more, as described above), positions 9, 13, 33, 51 and 57 and the C-terminal are solvent-accessible positions, and are preferably occupied by cysteines, which comprise thiol groups, which are the conjugation sites present in the protein building block, as described above. In addition, in this embodiment, the building block comprising or consisting of SEQ ID NO.: 105 (or any variant thereof with sequence identity of 80% or more, as described above) may additionally comprise an extra cysteine and/or an extra tyrosine at the N-terminal end of the polypeptide defined by SEQ ID NO.: 105 (or any variant thereof with sequence identity of 80% or more, as described above). If a cysteine is present at the N-terminal of the polypeptide defined by SEQ ID NO.: 105 (or any variant thereof with sequence identity of 80% or more, as described above), the cysteine may be followed by a flexible tag (sequence), such as (GG), (e.g., CGG-). If a tyrosine is present at the N-terminal of the polypeptide defined by SEQ ID NO.: 105 (or any variant thereof with sequence identity of 80% or more, as described above), the tyrosine may be followed by flexible tags (sequences), such as (GG) or (G₄S₁)₁₋₃GG tags (sequences) (e.g., YGG-, YGG(G₄S₁)₁₋₃-, YGG(S₁G₄)₁₋₃- or Y(G₄S₁)₁₋₃GG-), preferably YGG-, although longer linkers might be preferred for applications where, e.g., more flexibility is needed, as described in detail in Alan M. Marmelstein et al., Journal of the American Chemical Society, 2020, 142 (11), 5078-5086. In addition, the protein-based carrier building block which comprises, or alternatively, consists of, the polypeptide defined by SEQ ID NO.: 105 (or variants thereof with sequence identity of 80% or more, as described above) may additionally comprise a (Gly)₁₋₅ tag at the N-terminal end, to allow for conjugation of the cargo using sortase, as explained in detail above, see in particular Guimaraes C. P. et al., Theile C. S. et al. and Witte M. D. et al.

### Cargos

As described in detail above, the molecule of the present technology comprises at least one protein-based carrier building block which comprises at least one, preferably at least two, attachment point(s) or conjugation site(s) suitable for attachment of different molecules, including proteins, peptides, toxic payloads, nucleic acids, glycans, radio-isotopes, PEG, etc. and combinations thereof, see below for further examples of suitable cargos. In the context of the present technology, a “cargo” is any molecule which is/may be attached or conjugated to the protein-based carrier building block through the attachment point(s) or conjugation site(s) present therein. Hence, a “cargo”, in the context of the present technology, may be any molecule, including proteins, peptides, small molecules, toxic payloads, nucleic acids (such as DNA, RNA, siRNA, RNAi, etc.), vitamins, lipids, glycans, radio-isotopes, PEG, etc. and combinations thereof, see below for specific examples.

Hence, the molecule of the present technology may comprise at least one cargo as defined herein conjugated to one of the attachment points or conjugation sites present in the at least one protein-based carrier building block.

In a preferred embodiment, the at least one cargo which may be attached to the conjugation site(s) of the at least one carrier building block is an ISVD as described herein (also referred to in the present description as “cargo ISVD”). In this context, the cargo ISVD may preferably specifically bind to one or more proteins in the human body, such as human proteins and/or may also specifically bind other proteins present in the human body (e.g., viral or bacterial proteins which are in the human body).

In a preferred embodiment, the molecule of the present technology comprises at least one protein-based carrier building block and at least one cargo attached to one of the conjugation sites or attachment points present in the protein-based building block, preferably to a conjugation site or attachment point which is the side chain of an amino acid preferably located at a solvent-accessible position of the protein-based building block. In a preferred embodiment, the at least one protein-based carrier building block is an ISVD-derived building block, as described herein, and the cargo attached to the building block is an ISVD as described herein. Hence, in this embodiment, the molecule of the present technology comprises at least one protein-based carrier building block which is derived from an ISVD, preferably from a heavy-chain ISVD, and one cargo attached to it, wherein the cargo is an ISVD, preferably a heavy-chain ISVD.

In another embodiment, the at least one cargo which may be attached to the conjugation site(s) of the at least one carrier building block is a group, residue, moiety or binding unit which provides the protein-based carrier building block (and/or molecule) with increased (in vivo) half-life compared to the corresponding carrier building block/molecule without said one or more other groups, residues, moieties or binding units.

The cargos are attached (or “anchored”, “conjugated”, “linked”) to the at least one protein-based building block via the at least one conjugation site, as described above. The cargo(s) and the at least one protein-based building block may be directly linked to each other (as for example described in WO 1999/23221) and/or may be linked to each other via one or more suitable linkers, or any combination thereof. Suitable linkers for use in the molecule of the present technology will be clear to the skilled person, and may generally be any linker used in the art to link amino acid sequences or any other molecule comprised in the cargo. Preferably, said linker is suitable for use in constructing proteins or polypeptides that are intended for pharmaceutical use. Some particularly preferred linkers include the linkers that are used in the art to link antibody fragments or antibody domains. These include the linkers mentioned in the publication cited above, as well as for example linkers that are used in the art to construct diabodies or ScFv fragments (in this respect, however, it should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the molecule of this technology; this can be tuned depending on the specific applications, e.g., on the number and nature of the cargos to be attached to the protein-based building block, on the specific position and number of attachment points or conjugation sites and on the nature of the linker. As also shown in the Examples, the skilled person will be able to select an appropriate linker for a certain application).

For example, a linker may be a suitable amino acid or amino acid sequence, and in particular amino acid sequences of between 1 and 50, preferably between 1 and 30, such as between 1 and 10 amino acid residues. Some preferred examples of such amino acid sequences include Gly-Ser linkers, for example of the type (GlyₓSer_{y}) z, such as (for example (Gly₄Ser)₃ or (Gly₃Ser₂)₃, as described in WO 1999/42077 and the GS30, GS15, GS9 and GS7 linkers described in the applications by Ablynx mentioned herein (see for example WO 2006/040153 and WO 2006/122825), as well as hinge-like regions, such as the hinge regions of naturally occurring heavy chain antibodies or similar sequences (such as described in WO 1994/04678). Some linkers are depicted in Table A-1 below. For instance, a preferred linker which may be used in the molecule of the present technology is depicted in SEQ ID NO.: 163 (15GS).

Some other particularly preferred linkers are poly-alanine (such as AAA), as well as the linkers GS30 (SEQ ID NO: 85 in WO 2006/122825) and GS9 (SEQ ID NO: 84 in WO 06/122825). In a preferred aspect the linker is chosen from the group consisting of SEQ ID NOs: 158-169 and 193-196, and the linker “A3 (3A)” as depicted in table A-1 below, preferably SEQ ID NO: 163. Polyethylene glycol (PEG), in any of the variants described below, may also be used as a linker in the molecule of the present technology. Other suitable linkers for use in the molecule of the present technology are described, e.g., in Kjeldsen T. et al. (“Dually reactive long recombinant linkers for bioconjugations as an alternative to PEG”, ACS Omega, 2020, 5:19827-19833). As described therein polar protein sequences with PEG-like properties, sometimes called “recombinant PEG”, have in recent years been described by Alvarez (“Improving protein pharmacokinetics by genetic fusion to simple amino acid sequences”, J. Biol. Chem., 2004, 279:3375-3381), Amunix (mixed sequences of GEDSTAP residues, termed “ELNN polypeptides”, see, e.g., US 2014/0301974 A1), XL-protein (PAS repeats), Novo Nordisk (GQAP-like repeats), SOBI and others.

As used herein, the terms “ELNN polypeptides” and “ELNNs” are synonymous and refer to extended length polypeptides comprising non-naturally occurring, substantially non-repetitive sequences (e.g., polypeptide motifs) that are composed mainly of small hydrophilic amino acids, with the sequence having a low degree or no secondary or tertiary structure under physiologic conditions. ELNN polypeptides include unstructured hydrophilic polypeptides comprising repeating motifs of 6 natural amino acids (G, A, P, E, S, and/or T). In some embodiments, an ELNN polypeptide comprises multiple motifs of 6 natural amino acids (G, A, P, E, S, T), wherein the motifs are the same or comprise a combination of different motifs. In some embodiments, ELNN polypeptides can confer certain desirable pharmacokinetic, physicochemical, and pharmaceutical properties when linked to proteins (e.g., when linked to the protein-based building block of the present technology). Such desirable properties may include but are not limited to enhanced pharmacokinetic parameters and solubility characteristics, as well as improved therapeutic index. ELNN polypeptides are known in the art, and non-limiting descriptions relating to and examples of ELNN polypeptides known as XTEN® polypeptides are available in Schellenberger et al., (2009), Nat Biotechnol 27 (12): 1186-90; Brandl et al., (2020), Journal of Controlled Release 327:186-197; and Radon et al., (2021), Advanced Functional Materials 31, 2101633 (pages 1-33), the entire contents of each of which are incorporated herein by reference.

Ravtansine/soravtansine (N2′-Deacetyl-N2′-(4-mercapto-4-methyl-1-oxopentyl)-maytansine or DM4, CAS Registry Number: 796073-69-3) is a maytansinoid connected via a cleavable chemical linker to the targeting mAb which may be used as a cytotoxic component of, e.g., antibody-drug conjugates. This cleavable linker is also suitable for being used in the molecule of the present technology.

The length, the degree of flexibility and/or other properties of the linker(s) used may have some influence on the properties of the molecule of the present technology. Based on the disclosure herein and the disclosure of other publications, such as, for example, WO 2017/089618, the skilled person will be able to determine the optimal linker(s) for use in the specific molecule of the present technology, optionally after some limited routine experiments.

Further suitable linkers for use in the molecule of the present technology are, e.g., cleavable linkers, i.e., linkers which have a trigger in its structure that can be efficiently cleaved. For instance, Su, Z. et al. (“Antibody-drug conjugates: Recent advances in linker chemistry”, Acta Pharmaceutica Sinica B, 2021, 11 (12): 3889-3907) reviews linkers that may be comprised in antibody-drug conjugates and which may also be used in the molecule of the present technology. For example, suitable linkers for use in the molecule of the present technology are APN-maleimide linker (3-(4-(2,5-dioxo-2,5-dihydro-1H-pyrrol-1-yl)phenyl)propiolonitrile, MAPN) or bis-maleimido-PEG3 (BM(PEG)3) linker (BM(PEG)3 (1,11-bismaleimido-triethyleneglycol)).

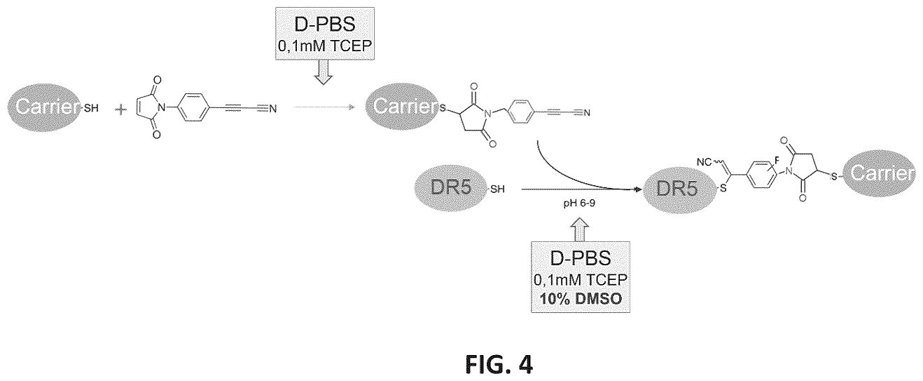

In addition, bifunctional linkers may be used. For instance, the APN-Maleimide linker (806536, Sigma-Aldrich) can be used. This linker allows for conjugation twice, via cysteine-based chemistry. Both APN and maleimide couple to free thiols, albeit at different speed, see FIG. 4 and Example 5.1 below. Other bifunctional linkers may also be used. For instance, a linker comprising (i) a maleimide group on one end and (ii) either a LPXTG motif (where X can be any amino acid and glycine cannot be a free carboxylate) as the sortase target or an oligoglycine (Gly₁₋₅) on the other end, as described in detail above.

When two or more linkers are used in the molecule of the present technology, these linkers may be the same or different. Again, based on the disclosure herein, the skilled person will be able to determine the optimal linkers for use in the specific molecule of the present technology, optionally after some limited routine experiments.

### (In Vivo) Half-Life Extending Moieties

As described above, the molecule of the present technology may comprise one or more other groups, residues, moieties or binding units, optionally linked via one or more linkers, such as peptide linkers, as defined above, in which said one or more other groups, residues, moieties or binding units provide the molecule of the present technology with increased (in vivo) half-life, compared to the corresponding molecule without said one or more other groups, residues, moieties or binding units (“(in vivo) half-life extending moiety”, or “half-life extending (HLE) moiety”). The HLE moiety is a cargo as described above when it is attached or conjugated to the at least one attachment point or conjugation site comprised in the protein-based carrier building block.

The term “half-life” as used here can generally be defined as described in paragraph o) on page 57 of WO 2008/020079 and as mentioned therein refers to the time taken for the serum concentration of the compound or polypeptide to be reduced by 50%, in vivo, for example due to degradation of the sequence or compound and/or clearance or sequestration of the sequence or compound by natural mechanisms. The in vivo half-life of the protein-based carrier building block and/or molecule comprising the protein-based carrier building block can be determined in any manner known per se, such as by pharmacokinetic analysis. Suitable techniques will be clear to the person skilled in the art and may for example generally be as described in paragraph o) on page 57 of WO 2008/020079. As also mentioned in paragraph o) on page 57 of WO 2008/020079, the half-life can be expressed using parameters such as the t_{1/2}-alpha, t_{1/2}-beta and the area under the curve (AUC). In this respect it should be noted that the term “half-life” as used herein in particular refers to the t_{1/2}-beta or terminal half-life (in which the t_{1/2}-alpha and/or the AUC or both may be kept out of considerations). Reference is for example made to the standard handbooks, such as Kenneth, A et al: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t_{1/2}-beta, either with or without an increase in the t_{1/2}-alpha and/or the AUC or both.

(In vivo) half-life can be extended by an increase in the hydrodynamic radius (size) or by a decrease in the molecule's clearance. For instance, (in vivo) half-life extending moieties such as polyethylene glycol or ELNN polypeptides increase the size of the molecules to which they are attached, therefore bypassing renal clearance, and thus increasing the half-life of those molecules. Other (in vivo) half-life extending moieties such as binding units that can bind to, e.g., serum albumin, increase the half-life of the molecules to which they are attached by binding, e.g., to serum albumin. Albumin is the most abundant plasma protein, is highly soluble, very stable and has an extraordinarily long circulatory half-life as a direct result of its size and interaction with the FcRn mediated recycling pathway, see, e.g., Sleep D. et al., “Albumin as a versatile platform for drug half-life extension”, Biochim Biophys Acta, 2013, 1830 (12): 5526-34.

The type of groups, residues, moieties or binding units is not generally restricted and may for example be chosen from the group consisting of a polyethylene glycol (PEG) molecule, ELNN polypeptides or fragments thereof, as described above, serum proteins or fragments thereof, binding units that can bind to serum proteins, an Fc portion, and small proteins or peptides that can bind to serum proteins.

More specifically, said one or more other groups, residues, moieties or binding units that provide the molecule of the present technology with increased half-life can be chosen from the group consisting of a polyethylene glycol (PEG) molecule, ELNN polypeptides or fragments thereof, binding units that can bind to serum albumin, such as human serum albumin, or a serum immunoglobulin, such as IgG, or Fc fusions which might provide extra functionalities in vivo such as HLE via FcRn, immune effector functions via Fcγ receptors. In one embodiment, said one or more other groups, residues, moieties or binding units that provide the molecule of the present technology with increased half-life is a binding unit that can bind to human serum albumin. In one embodiment, the binding unit is an ISVD.

For example, WO 2004/041865 describes ISVDs binding to serum albumin (and in particular against human serum albumin) that can be linked or attached to other proteins (such as one or more building blocks of the present technology) in order to increase the half-life of the molecule of the present technology.

The international application WO 2006/122787, the content of which is herein incorporated by reference, describes a number of ISVDs against (human) serum albumin. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of therapeutic proteins and polypeptides, and other entities or moieties, such as the molecule of the present technology.

Moreover, WO 2012/175400, the content of which is herein incorporated by reference, describes a further improved version of Alb-1, called Alb-23.

In one embodiment, the molecule of the present technology further comprises a serum albumin binding moiety selected from Alb-1, Alb-3, Alb-4, Alb-5, Alb-6, Alb-7, Alb-8, Alb-9, Alb-10 (described in WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO 2015/173325, WO 2017/080850, WO 2017/085172, WO 2018/104444, WO 2018/134235, and WO 2018/134234, the content of which is herein incorporated by reference. Some serum albumin binders are also shown in Table 8 below.

In one embodiment, the molecule of the present technology comprises the serum albumin binding moiety Alb23 (SEQ ID NO.: 51) as defined in Table 8 below. In one preferred embodiment, the molecule of the present technology comprises the serum albumin binding moiety Alb23002 (SEQ ID NO.: 63) as defined in Table 8 below. In another preferred embodiment, the molecule of the present technology comprises the serum albumin binding moiety Alb23002(E1D) (SEQ ID NO.: 106) as defined in Table 8 below.

TABLE 8
Serum albumin binding ISVD sequences (″ID″ refers to the SEQ ID NO as used herein)

| Name | ID | Amino acid sequence |
|---|---|---|
| Alb8 | 50 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVTVSS |
| Alb23 | 51 | EVQLLESGGGLVQPGGSLRLSCAASGFTFRSFGMSWVRQAPGKGPE |
| | | WVSSISGSGSDTLYADSVKGRFTISRDNSKNTLYLQMNSLRPEDTAVY |
| | | YCTIGGSLSRSSQGTLVTVSS |
| Alb129 | 52 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTATYY |
| | | CTIGGSLSRSSQGTLVTVSSA |
| Alb132 | 53 | EVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTATY |
| | | YCTIGGSLSRSSQGTLVTVSSA |
| Alb11 | 54 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVTVSS |
| Alb11(S112K)-A | 55 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVKVSSA |
| Alb82 | 56 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSS |
| Alb82-A | 57 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSA |
| Alb82-AA | 58 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSAA |
| Alb82-AAA | 59 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSAAA |
| Alb82-G | 60 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSG |
| Alb82-GG | 61 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSGG |
| Alb82-GGG | 62 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALYY |
| | | CTIGGSLSRSSQGTLVTVSSGGG |
| Alb23002 | 63 | EVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTALYY |
| | | CTIGGSLSRSSQGTLVTVSS |
| Alb223 | 64 | EVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTALYY |
| | | CTIGGSLSRSSQGTLVTVSSA |
| Alb23002(E1D) | 106 | DVQLVESGGGVVQPGGSLRLSCAASGFTFRSFGMSWVRQAPGKGPE |
| | | WVSSISGSGSDTLYADSVKGRFTISRDNSKNTLYLQMNSLRPEDTALYY |
| | | CTIGGSLSRSSQGTLVTVSS |

In one embodiment, the molecule of the present technology comprises a HLE moiety as described in the following item A:
A. An ISVD that binds to human serum albumin and comprises
   i. a CDR1 that is the amino acid sequence of SEQ ID NO: 65 or an amino acid sequence with 2 or 1 amino acid difference with SEQ ID NO: 65;
   ii. a CDR2 that is the amino acid sequence of SEQ ID NO: 66 or an amino acid sequence with 2 or 1 amino acid difference with SEQ ID NO: 66; and
   iii. a CDR3 that is the amino acid sequence of SEQ ID NO: 67 or an amino acid sequence with 2 or 1 amino acid difference with SEQ ID NO: 67.

In one embodiment, the ISVD comprises a CDR1 that is the amino acid sequence of SEQ ID NO: 65, a CDR2 that is the amino acid sequence of SEQ ID NO: 66 and a CDR3 that is the amino acid sequence of SEQ ID NO: 67.

Examples of such an ISVD that binds to human serum albumin have one or more, or all, framework regions as indicated for construct ALB23002 (SEQ ID NO.: 63) in Tables 9 and 10 (in addition to the CDRs as defined in the preceding item A). In one embodiment, it is an ISVD comprising or consisting of the full amino acid sequence of construct ALB23002 (SEQ ID NO: 63).

TABLE 9
Sequences for CDRs according to AbM CDR and framework annotation (″ID″ refers
to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 63 | ALB23002 | 69 | EVQLVE | 65 | GFTF | 70 | WVRQ | 66 | SISGS | 71 | YADSVK | 67 | GGSLSR | 72 | SSQGT |
| | | | SGGGV | | RSFG | | APGK | | GSDT | | GRFTISR | | | | LVTVS |
| | | | VQPGG | | MS | | GPEW | | L | | DNSKNT | | | | S |
| | | | SLRLSC | | | | VS | | | | LYLQMN | | | | |
| | | | AAS | | | | | | | | SLRPEDT | | | | |
| | | | | | | | | | | | ALYYCTI | | | | |

TABLE 10
Sequences for CDRs according to Kabat CDR and frameworks annotation (″ID″
refers to the given SEQ ID NO)

| ID | V_{HH} | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 63 | ALB23002 | 73 | EVQLVES | 74 | SFGM | 75 | WVRQA | 76 | SISGS | 77 | RFTISRDNSK | 78 | GGSLSR | 79 | SSQG |
| | | | GGGVVQ | | S | | PGKGPE | | GSDT | | NTLYLQMNSL | | | | TLVT |
| | | | PGGSLRLS | | | | WVS | | LYAD | | RPEDTALYYC | | | | VSS |
| | | | CAASGFT | | | | | | SVKG | | TI | | | | |
| | | | FR | | | | | | | | | | | | |

Item A′ can be also described using the Kabat CDR definition as:
A′. An ISVD that binds to human serum albumin and comprises
   i. a CDR1 that is the amino acid sequence of SEQ ID NO: 74 or an amino acid sequence with 2 or 1 amino acid difference with SEQ ID NO: 74;
   ii. a CDR2 that is the amino acid sequence of SEQ ID NO: 76 or an amino acid sequence with 2 or 1 amino acid difference with SEQ ID NO: 76; and
   iii. a CDR3 that is the amino acid sequence of SEQ ID NO: 78 or an amino acid sequence with 2 or 1 amino acid difference with SEQ ID NO: 78.

In one embodiment, the ISVD comprises a CDR1 that is the amino acid sequence of SEQ ID NO: 74, a CDR2 that is the amino acid sequence of SEQ ID NO: 76 and a CDR3 that is the amino acid sequence of SEQ ID NO: 78.

Examples of such an ISVD that binds to human serum albumin have one or more, or all, framework regions as indicated for construct ALB23002 in Table 10 (in addition to the CDRs as defined in the preceding item A′). In one embodiment, it is an ISVD comprising or consisting of the full amino acid sequence of construct ALB23002 (SEQ ID NO: 63, see also Table 10).

Also in another embodiment, the amino acid sequence of an ISVD binding to human serum albumin may have a sequence identity of more than 90%, such as more than 95% or more than 99%, with SEQ ID NO: 63, wherein the CDRs are as defined in the preceding item A or A′. In one embodiment, the ISVD binding to human serum albumin comprises or consists of the amino acid sequence of SEQ ID NO: 63.

When such an ISVD binding to human serum albumin has 2 or 1 amino acid difference in at least one CDR relative to a corresponding reference CDR sequence (item A or A′ above), the ISVD has at least half the binding affinity, or at least the same binding affinity, to human serum albumin compared to construct ALB23002 (SEQ ID NO: 63), wherein the binding affinity is measured using the same method, such as SPR.

In one embodiment, when such an ISVD binding to human serum albumin has a C-terminal position, it exhibits a C-terminal extension, such as a C-terminal alanine, cysteine, or glycine extension. In one embodiment such an ISVD is selected from SEQ ID Nos: 52, 53, 55, 57, 58, 59, 60, 61, 62, and 64 (see table 8 above). In another embodiment, the ISVD binding to human serum albumin has another position than the C-terminal position (i.e. is not the C-terminal ISVD of the molecule of the present technology). In one embodiment such an ISVD is selected from SEQ ID Nos: 63, 50, 51, 54, 56 and 106 (see Table 8 above).

In one embodiment, said one or more other groups, residues, moieties or binding units that provide the molecule with increased half-life is a peptide that can bind to human serum albumin.

In particular the “serum-albumin binding polypeptide or binding domain” may be any suitable serum-albumin binding peptide capable of increasing the half-life (preferably T_{1/2}ß, as defined above) of the molecule (compared to the same molecule without the serum-albumin binding peptide or binding domain).

Specifically, the polypeptide sequence suitable for extending serum half-life is a polypeptide sequence capable of binding to a serum protein with a long serum half-life, such as serum albumin, transferrin, IgG, etc, in particular serum albumin.

Polypeptide sequences capable of binding to serum albumin have previously been described and may in particular be serum albumin binding peptides as described in WO 2008/068280 (and in particular WO 2009/127691 and WO 2011/095545), the content of which is herewith incorporated by reference.

In one embodiment, said one or more other groups, residues, moieties or binding units that provide the molecule with increased half-life is straight or branched chain poly (ethylene glycol) (PEG) or derivatives thereof (such as methoxypoly (ethylene glycol) or mPEG), which increase the hydrodynamic radius of the molecule, thus exceeding the renal clearance and hence rendering the molecule with tuneable half-life extension). Generally, any suitable form of PEGylation can be used, such as the PEGylation used in the art for antibodies and antibody fragments (including but not limited to domain antibodies and scFv's); reference is made, for example, to: Chapman, Nat. Biotechnol., 54, 531-545 (2002); Veronese and Harris, Adv. Drug Deliv. Rev. 54, 453-456 (2003); Harris and Chess, Nat. Rev. Drug. Discov. 2 (2003); WO 04/060965; and U.S. Pat. No. 6,875,841. Various reagents for PEGylation of polypeptides are also commercially available, for example from Nektar Therapeutics, USA, or NOF Corporation, Japan, such as the Sunbright® EA Series, SH Series, MA Series, CA Series, and ME Series, such as Sunbright® ME-100MA, Sunbright® ME-200MA, and Sunbright® ME-400MA.

After covalent attachment of PEG, molecules can have prolonged blood circulation half-lives, improved drug solubility and stability, and reduced immunogenicity (Swierczewskaa M., et al., “What is the future of PEGylated therapies?”, Expert Opin Emerg Drugs. 2015; 20 (4): 531-536).

The PEG may be linear or branched and have a molecular weight from about 1 to about 40 kDa, such as from about 1 to about 30 kDa, or from about 1 to about 20 kDa, or from about 1 to about 10 kDa, preferably from about 2 to about 7 kDa, more preferably from about 4 to about 6 k Da and even more preferably of about 5 kDa. The smaller PEG size (e.g., 5 kDa) should enable renal clearance of the PEG moieties, thus bypassing the disadvantages of standard used large 40-60 kDa PEG. In one embodiment, the protein-based carrier building block of the present technology comprises more than one PEG molecules as described above. For instance, the protein-based carrier building block of the present technology may comprise 2, 3, 4, 5, 6, 7, 8 or more PEG molecules, such as from 4 to 8 PEG molecules, such as from 5 to 7 PEG molecules, such as 6 PEG molecules. In one embodiment, the protein-based carrier building block comprises 6 molecules of linear 5 kDa PEG. Suitable PEG-groups and methods for attaching them to the protein-based carrier building block will be clear to the skilled person.

High MW PEG accumulate in the circulation and cannot be efficiently renal cleared. This may have a toxic effect in the human body. Conversely, PEG with lower MW can be renal clearance, reducing the toxicity of the PEG-comprising molecules. See, e.g., Fang J L. et al., “Toxicity of high-molecular-weight polyethylene glycols in Sprague Dawley rats”, Toxicol Lett., 2022, 359:22-30.

For instance, the present technology provides one molecule as described herein which comprises at least one ISVD-based building block, as defined herein, onto which at least one PEG molecule is conjugated, preferably with a MW of less than 20 kDa, such as less than 15 kDa, or less than 10 kDa, or less than 7.5 kDa, such as about 5 kDa, or less, more preferably 5 kDa or less, attached to at least one attachment point or conjugation site. It is preferred that the PEG molecule attached or conjugated to the protein-based building block has a low MW, so that it can be efficiently cleared by the kidneys. The skilled person would understand that, if a PEG molecule has a high MW, it may not be efficiently renal cleared. Thus, it is preferred that the PEG molecule(s) comprised in the protein-based building block has (have) low MW, as explained herein (e.g., less than 20 kDa, preferably less than 10 kDa, or less than 7.5 kDa, or less than 5 kDa, or even 1-5 kDa. Of course, as the skilled person would understand, the chosen size of the PEG molecules also depends on the number of PEG molecules attached to the protein-based carrier building block.

For instance, the ISVD-based building block comprises or, alternatively, consists of a building block selected from SEQ ID NOs.: 80-95, 175, 185, 186, 206 or 222-224, or a sequence which has 80% or more sequence identity with SEQ ID NO.: 80-95, 175, 185, 186, 206 or 222-224, preferably a sequence which has 85% or more, 90% or more, 95% or more, 97% or more or 99% or more sequence identity with SEQ ID NO.: 80-95, 175, 185, 186, 206 or 222-224, provided that the building block has a globular 3D structure, is soluble, has a size (molecular mass) of about 2.5 to about 70 kDa, such about 2.5 to about 50 kDa, or of as about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one cargo attached to it through the at least one conjugation site or attachment point, does not specifically bind to any non-protein molecule and/or does not specifically bind to any non-human protein to which the ISVD precursor specifically binds.

As mentioned, other means of increasing the half-life of the molecule of the present technology (such as the presence of linear or branched 40-60 kDa PEGylation, or fusion to human albumin or a suitable fragment thereof), although less preferred, are also included in the scope of the technology.

For instance, the present technology provides one molecule as described herein which comprises at least one DARPin-based building block, as defined herein, onto which at least one PEG molecule is conjugated, preferably with a MW of less than 20 kDa, such as less than 15 kDa, or less than 10 kDa, or less than 7.5 kDa, such as about 5 kDa, or less, more preferably 5 kDa, attached to at least one attachment point or conjugation site. For instance, the ISVD-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, onto which at least one PEG molecule is conjugated, preferably with a MW of less than 20 kDa, such as less than 15 kDa, or less than 10 kDa, or less than 7.5 kDa, such as about 5 kDa, or less, more preferably 5 kDa, attached to at least one attachment point or conjugation site.

For instance, the present technology provides one molecule as described herein which comprises at least one building block based on a small globular protein, such as CKS1, as defined herein, onto which at least one PEG molecule is conjugated, preferably with a MW of less than 20 kDa, such as less than 15 kDa, or less than 10 kDa, or less than 7.5 kDa, such as about 5 kDa, or less, more preferably 5 kDa, attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one ISVD-based building block with SEQ ID NO.: 90, onto which at least one, preferably more than one, such as two, or, preferably, three PEG molecules, preferably with a MW of less than 20 kDa, such as less than 15 kDa, or less than 10 kDa, or less than 7.5 kDa, such as about 5 kDa, or less, more preferably 5 kDa, are conjugated to the attachment points or conjugation sites of the ISVD-based building block. In some embodiments, the molecule further comprises a HLE moiety, such as an albumin-binding ISVD, e.g., SEQ ID NO.: 106. In some embodiments, the at least one PEG molecule, preferably with a MW of less than 20 kDa, such as less than 15 kDa, or less than 10 kDa, or less than 7.5 kDa, such as about 5 kDa, or less, more preferably 5 kDa, is conjugated to the ISVD-based building block comprised in molecule T028100118 (SEQ ID NO.: 117). As described above, there are preferably more than one PEG molecules, preferably with a MW of less than 20 kDa, such as less than 15 kDa, or less than 10 kDa, or less than 7.5 kDa, such as about 5 kDa, or less, more preferably 5 kDa, conjugated to the ISVD-derived building blocks, preferably 3 PEG molecules, preferably with a MW of less than 20 kDa, such as less than 15 kDa, or less than 10 kDa, or less than 7.5 kDa, such as about 5 kDa, or less, more preferably 5 kDa, per ISVD-based building block.

As shown in the examples, molecules comprising two or more PEG molecules with lower molecular weight (e.g., 5 kDa) are able to increase the half-life of the molecules, see Example 15.

Generally, when the protein-based carrier building block and/or molecule of the present technology has increased half-life (e.g. through the presence of a half-life increasing ISVD, PEG moieties or any other suitable way of increasing half-life, as described above), the resulting protein-based carrier building block and/or molecule preferably has a half-life (as defined herein) that is at least 2 times, preferably at least 5 times, for example at least 10 times or more, such at least 20 times, or at least 50 times, or at least 100 times, or at least 150 times, or at least 200 times, or at least 300 times, or at least 400 times, or at least 500 times, greater than the half-life of the protein-based carrier building block and/or molecule without the half-life increasing group, residue, moiety or binding unit (as measured either in man and/or a suitable animal model, such as mouse or cynomolgus monkey). In particular, the protein-based carrier building block and/or molecule may have a half-life (as defined herein) in human subjects of at least 1 day, such as at least 3 days, or at least 7 days, such as at least 10 days, or at least 15 days, or at least 20 days. The skilled person is able to select the HLE moiety based on the desired half-life of the protein-based carrier building block and/or molecule of the present technology. For certain applications, however, it may be desirable that the protein-based carrier building block and/or molecule of the present technology has shorter half-life (e.g., radio imaging/therapy in a theranostic setting).

In one embodiment, the molecule of the present technology may comprise a half-life extending moiety, as described above, attached or conjugated to the protein-based carrier building block. For instance, the half-life extending moiety may be an albumin-binding ISVD, such as an albumin-binding ISVD as defined in Table 8 above, or a polypeptide which has at least 80% identity with a polypeptide of Table 8, preferably at least 85%, more preferably at least 90%, or at least 95%, or at least 99% identity with a polypeptide of Table 8. For instance, the molecule according to this embodiment may comprise a half-life extending moiety which is the polypeptide with SEQ ID NO.: 106, or the polypeptide with SEQ ID NO.: 63, and one protein-based carrier building block.

The protein-based carrier building block in this embodiment may be any protein-based building block as described above in this specification. In particular, the protein-based carrier building block in this embodiment may be an ISVD-based building block, as described above, a DARPin-based building block or a small globular human protein-based building block (e.g., a CKS-1-based building block), as described above.

For instance, the protein-based carrier building block in this embodiment may be an ISVD-derived protein-based carrier building block as defined in any one of SEQ ID NO.: 80-95, 175, 185-186, 206, 222-224 or a polypeptide which has at least 80% identity with a polypeptide as defined in any one of SEQ ID NO.: 80-95 or 175, preferably at least 85%, more preferably at least 90%, or at least 95%, or at least 99% identity with a polypeptide as defined in any one of SEQ ID NO.: 80-95 or 175.

For instance, the protein-based carrier building block in this embodiment may be derived from a small globular protein, such as a protein based on cyclin-dependent kinase subunit (CKS). For instance, the protein-based carrier building block in this embodiment may be a polypeptide as defined in any one of SEQ ID NO.: 99-105, 205 or 191-192, or a polypeptide which has at least 80% identity with a polypeptide as defined in any one of SEQ ID NO.: 99-105, 205 or 191-192, preferably at least 85%, more preferably at least 90%, or at least 95%, or at least 99% identity with a polypeptide as defined in any one of SEQ ID NO.: 99-105, 205 or 191-192.

For instance, the protein-based carrier building block in this embodiment may be derived from a DARPin protein. For instance, the protein-based carrier building block in this embodiment may be a protein as defined in any one of SEQ ID NO.: 96-98, 181-182, 199, 208 or 188-189, or a polypeptide which has at least 80% identity with a polypeptide as defined in any one of SEQ ID NO.: 96-98, 181-182, 199, 208 or 188-189, preferably at least 85%, more preferably at least 90%, or at least 95%, or at least 99% identity with a polypeptide as defined in any one of SEQ ID NO.: 96-98, 181-182, 199, 208 or 188-189.

Hence, the present technology further provides a molecule comprising or, alternatively consisting of, at least one protein-based building block as described herein and at least one (in vivo) half-life extending moiety as described herein.

For instance, the molecule of the present technology may comprise (i) a protein-based building block comprising or, alternatively, consisting of SEQ ID NO.: 80-95, 175, 185-186, 206, 222-224 or a polypeptide which has at least 80% identity with a polypeptide as defined in any one of SEQ ID NO.: 80-95, 175, 185-186, 206, 222-224 preferably at least 85%, more preferably at least 90%, or at least 95%, or at least 99% identity with a polypeptide as defined in any one of SEQ ID NO.: 80-95, 175, 185-186, 206, 222-224 and (ii) a half-life extending moiety as described herein, such as a serum albumin binding ISVD, e.g., as defined in Table 8, and/or a PEG molecule, and/or a ELNN polypeptide or a fragment thereof.

For instance, the molecule of the present technology may comprise (i) a protein-based building block comprising or, alternatively, consisting of SEQ ID NO.: 96-98, 181-182, 199, 208 or 188-189, or a polypeptide which has at least 80% identity with a polypeptide as defined in any one of SEQ ID NO.: 96-98, 181-182, 199, 208 or 188-189, preferably at least 85%, more preferably at least 90%, or at least 95%, or at least 99% identity with a polypeptide as defined in any one of SEQ ID NO.: 96-98, 181-182, 199, 208 or 188-189 and (ii) a half-life extending moiety as described herein, such as a serum albumin binding ISVD, e.g., as defined in Table 8, and/or a PEG molecule, and/or a ELNN polypeptide or a fragment thereof.

For instance, the molecule of the present technology may comprise (i) a protein-based building block comprising or, alternatively, consisting of SEQ ID NO.: 99-105, 205 or 191-192, or a polypeptide which has at least 80% identity with a polypeptide as defined in any one of SEQ ID NO.: 99-105, 205 or 191-192, preferably at least 85%, more preferably at least 90%, or at least 95%, or at least 99% identity with a polypeptide as defined in any one of SEQ ID NO.: 99-105, 205 or 191-192 and (ii) a half-life extending moiety as described herein, such as a serum albumin binding ISVD, e.g., as defined in Table 8, and/or a PEG molecule, and/or a ELNN polypeptide or a fragment thereof.

In one preferred embodiment, the molecule of the present technology comprises the serum albumin binding moiety Alb23002 (SEQ ID NO.: 63) as defined in Table 8. In another preferred embodiment, the molecule of the present technology comprises the serum albumin binding moiety Alb23002 (E1D) (SEQ ID NO.: 106) as defined in Table 8.

The half-life extending moiety may be covalently attached to the conjugation site on the protein-based carrier building block either directly or by means of a linker, such as a linker selected from the linkers depicted in Table A-1 (SEQ ID NO.: 158-169 and 193-196). For instance, the half-life extending moiety may be covalently attached to the protein-based carrier building block by means of a linker, such as a 15GS linker (SEQ ID NO.: 163).

Examples of polypeptides comprising a half-life extending moiety which is an albumin-binding ISVD and a protein-based carrier building block which is based on an ISVD are depicted in SEQ ID NOs.: 107-122 and 176, see Table 11 below.

TABLE 11
Examples of polypeptides comprising a half-life extending moiety which is an
albumin-binding ISVD and a protein-based carrier building block which is based on an ISVD
(″ID″ refers to the SEQ ID NO as used herein)

| Polypeptide | ID | albumin-binding ISVD | ID | Linker | ID | ISVD-based building block | ID |
|---|---|---|---|---|---|---|---|
| T028100069 | 107 | DVQLVESGGGVVQPGGSL | 106 | GGGGSGGG | 163 | EVQLVESGGGLVQAGGSLSISCAASG | 80 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGDITIGPPNVEGRFTISRDNAKN | |
| | | DTLYADSVKGRFTISRDNSK | | | | TGYLQMNSLAPDDTAVYYCGAGTPL | |
| | | INTLYLQMNSLRPEDTALYY | | | | NPGAYIYCWSYDYWGCGTLVTVSS | |
| | | CTIGGSLSRSSQGTLVTVSS | | | | | |
| T028100070 | 108 | DVQLVESGGGVVQPGGSL | 106 | GGGGSGGG | 163 | EVQLVESGGGLVQAGGSLSISCAASG | 81 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGDITIGPPNVEGRFTISRDNACN | |
| | | DTLYADSVKGRFTISRDNSK | | | | TGYLQMNSLAPDDTAVYYCGAGTPL | |
| | | NTLYLQMNSLRPEDTALYY | | | | NPCAYIYDWSYDYWGRGTLVTVSS | |
| | | CTIGGSLSRSSQGTLVTVSS | | | | | |
| T028100071 | 109 | DVQLVESGGGVVQPGGSL | 106 | GGGGSGGG | 163 | EVQLVESGGGLVQAGGSLSICCAASG | 82 |
| | | RLSCAASGFTFRSFGMSWV | | GSGGGGS | | GSLSNYVLGWFRQAPGKEREFVAAI | |
| | | RQAPGKGPEWVSSISGSGS | | | | NWRGDITIGPPNVEGRFCISRDNAK | |
| | | DTLYADSVKGRFTISRDNSK | | | | NTGYLQMNSLAPDDTAVYYCGAGTP | |
| | | NTLYLQMNSLRPEDTALYY | | | | LNPGAYIYCWSYDYWGRGTLVTVSS | |
| | | CTIGGSLSRSSQGTLVTVSS | | | | | |
| T028100072 | 110 | DVQLVESGGGVVQPGGSL | 106 | GGGGSGGG | 163 | EVQLVESGGGLVCAGGSLSISCAASG | 83 |
| | | RLSCAASGFTFRSFGMSWV | | GSGGGGS | | GSLSNYVLGWFRQAPGKEREFVAAI | |
| | | RQAPGKGPEWVSSISGSGS | | | | NWRGDITIGPPNVEGRFTISRCNAKN | |
| | | DTLYADSVKGRFTISRDNSK | | | | TGYLQMNSLAPDDTAVYYCGAGTPL | |
| | | NTLYLQMNSLRPEDTALYY | | | | NPCAYIYDWSYDYWGRGTLVTVSS | |
| | | CTIGGSLSRSSQGTLVTVSS | | | | | |
| T028100073 | 111 | DVQLVESGGGVVQPGGSL | 106 | GGGGSGGG | 163 | EVQLVESGGGLVCAGGSLSISCAASG | 84 |
| | | RLSCAASGFTFRSFGMSWV | | GSGGGGS | | GSLSCYVLGWFRQAPGKEREFVAAIN | |
| | | RQAPGKGPEWVSSISGSGS | | | | WRGDITIGPPNVEGRFTISRDNAKNT | |
| | | DTLYADSVKGRFTISRDNSK | | | | GYLQMNSLAPDDTAVYYCGAGTPLN | |
| | | NTLYLQMNSLRPEDTALYY | | | | PGAYIYCWSYDYWGRGTLVTVSS | |
| | | CTIGGSLSRSSQGTLVTVSS | | | | | |
| T028100074 | 112 | DVQLVESGGGVVQPGGSL | 106 | GGGGSGGG | 163 | EVQLVECGGGLVQAGGSLSISCAASG | 85 |
| | | RLSCAASGFTFRSFGMSWV | | GSGGGGS | | GSLSNYVLGWFRQAPGKCREFVAAI | |
| | | RQAPGKGPEWVSSISGSGS | | | | NWRGCITIGPPNVEGRFTISRDNAKN | |
| | | DTLYADSVKGRFTISRDNSK | | | | TGYLQMNSLAPDDTAVYYCGAGTPL | |
| | | NTLYLQMNSLRPEDTALYY | | | | NPGAYIYDWSYDYWGRGTLVTVSS | |
| | | CTIGGSLSRSSQGTLVTVSS | | | | | |
| T028100078 | 113 | DVQLVESGGGVVQPGGSL | 106 | GGGGSGGG | 163 | EVQLVESGGGLVQAGGSLCISCAASG | 86 |
| | | RLSCAASGFTFRSFGMSWV | | GSGGGGS | | GSLSNYVLGWFRQAPGKCREFVAAI | |
| | | RQAPGKGPEWVSSISGSGS | | | | NWRGDITIGPPNVECRFTICRDNAKN | |
| | | DTLYADSVKGRFTISRDNSK | | | | TGYLQMNCLAPDDTAVYYCGAGTPL | |
| | | NTLYLQMNSLRPEDTALYY | | | | NPGAYIYDWSYDYWGRGTLVTVCS | |
| | | CTIGGSLSRSSQGTLVTVSS | | | | | |
| T028100079 | 114 | DVQLVESGGGVVQPGGSL | 106 | GGGGSGGG | 163 | EVQLVESGGGLVQAGGSLSICCAASG | 87 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGCITIGPPNVEGRFCISRDNCKN | |
| | | DTLYADSVKGRFTISRDNSK | | | | TGYLQMNSLAPDDTAVYYCGAGTPL | |
| | | NTLYLQMNSLRPEDTALYY | | | | NPGAYIYDWSYDYWGRGTLVTVCS | |
| | | CTIGGSLSRSSQGTLVTVSS | | | | | |
| T028100080 | 115 | DVQLVESGGGVVQPGGSL | 106 | GGGGSGGG | 163 | EVQLVESGGGLVQAGGSLCISCCASG | 88 |
| | | RLSCAASGFTFRSFGMSWV | | GSGGGGS | | GSLSCYVLGWFRQAPGKEREFVAAIN | |
| | | RQAPGKGPEWVSSISGSGS | | | | WRGDITIGPPNVEGRFTICRDNAKNT | |
| | | DTLYADSVKGRFTISRDNSK | | | | GYLQMNCLAPDDTAVYYCGAGTPLN | |
| | | NTLYLQMNSLRPEDTALYY | | | | PGAYIYCWSYDYWGRGTLVTVSS | |
| | | CTIGGSLSRSSQGTLVTVSS | | | | | |
| T028100081 | 116 | DVQLVESGGGVVQPGGSL | 106 | GGGGSGGG | 163 | EVQLVESGGGLVCAGGSLSISCAACG | 89 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGDITIGPPNVECRFTISRCNAKN | |
| | | DTLYADSVKGRFTISRDNSK | | | | TGYLQMNSLAPDDTAVYYCGAGTPL | |
| | | NTLYLQMNSLRPEDTALYY | | | | NPCAYIYDWSYDYWGRGTLVTVSS | |
| | | CTIGGSLSRSSQGTLVTVSS | | | | | |
| T028100118 | 117 | EVQLVESGGGVVQPGGSL | 63 | GGGGSGGG | 163 | EVQLVESGGGVVQAGGSLSISCAAC | 90 |
| | | RLSCAASGFTFRSFGMSWV | | GSGGGGS | | GGSLSNYVLGWFRQAPGCEREFVAA | |
| | | RQAPGKGPEWVSSISGSGS | | | | INWRGDITIGPPNVEGRFTISRDNAC | |
| | | DTLYADSVKGRFTISRDNSK | | | | NTGYLQMNCLAPDDTALYYCGAGTP | |
| | | NTLYLQMNSLRPEDTALYY | | | | LNPCAYIYDWSYDYWGRGTLVTVCS | |
| | | CTIGGSLSRSSQGTLVTVSS | | | | | |
| T028100119 | 118 | EVQLVESGGGVVQPGGSL | 63 | GGGGSGGG | 163 | EVQLVESGGGVVQAGGSLSISCAAC | 91 |
| | | RLSCAASGFTFRSFGMSWV | | GSGGGGS | | GGSLSNYVLGWFRQAPGCEREFVAA | |
| | | RQAPGKGPEWVSSISGSGS | | | | INWRGDITIGPPNVEGRFTISRDNAC | |
| | | DTLYADSVKGRFTISRDNSK | | | | NTGYLQMNSLAPDDTALYYCGAGTP | |
| | | INTLYLQMNSLRPEDTALYY | | | | LNPCAYIYDWSYDYWGCGTLVTVCS | |
| | | CTIGGSLSRSSQGTLVTVSS | | | | | |
| T028100120 | 119 | EVQLVESGGGVVQPGGSL | 63 | GGGGSGGG | 163 | EVQLVESGGGVVQAGGSLSISCAAC | 92 |
| | | RLSCAASGFTFRSFGMSWV | | GSGGGGS | | GGSLSNYVLGWFRQAPGCEREFVAA | |
| | | RQAPGKGPEWVSSISGSGS | | | | INWRGDITIGPPNVEGRFTISRDNAC | |
| | | DTLYADSVKGRFTISRDNSK | | | | NTGYLQMNSLAPDDTALYYCGAGTP | |
| | | NTLYLQMNSLRPEDTALYY | | | | LNPCAYIYDWSYDYWGCGTLVTVSS | |
| | | CTIGGSLSRSSQGTLVTVSS | | | | GGC | |
| T028100121 | 120 | EVQLVESGGGVVQPGGSL | 63 | GGGGSGGG | 163 | EVQLVESGGGVVQAGGSLSISCAASG | 93 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGDITIGPPNVEGRFCISRDNAC | |
| | | DTLYADSVKGRFTISRDNSK | | | | NTGYLQMNSLAPDDTALYYCGAGTP | |
| | | INTLYLQMNSLRPEDTALYY | | | | LNPCAYIYDWSYDYWGCGTLVTVSS | |
| | | CTIGGSLSRSSQGTLVTVSS | | | | GGC | |
| T028100122 | 121 | EVQLVESGGGVVQPGGSL | 63 | GGGGSGGG | 163 | EVQLVESGGGVVQAGGSLSISCAAC | 94 |
| | | RLSCAASGFTFRSFGMSWV | | GSGGGGS | | GGSLSNYVLGWFRQAPGCEREFVAA | |
| | | RQAPGKGPEWVSSISGSGS | | | | INWRGDITIGPPNVEGRFTISRDNAC | |
| | | DTLYADSVKGRFTISRDNSK | | | | NTGYLQMNSLAPDDTALYYCGAGTP | |
| | | NTLYLQMNSLRPEDTALYY | | | | LNPGAYIYCWSYDYWGCGTLVTVSS | |
| | | CTIGGSLSRSSQGTLVTVSS | | | | GGC | |
| T028100123 | 122 | EVQLVESGGGVVQPGGSL | 63 | GGGGSGGG | 163 | EVQLVESGGGVVQAGGSLSISCAASG | 95 |
| | | RLSCAASGFTFRSFGMSWV | | GSGGGGS | | GSLSNYVLGWFRQAPGCEREFVAAI | |
| | | RQAPGKGPEWVSSISGSGS | | | | NWRGDITIGPPNVEGRFCISRDNAC | |
| | | DTLYADSVKGRFTISRDNSK | | | | INTGYLQMNSLAPDDTALYYCGAGTP | |
| | | NTLYLQMNSLRPEDTALYY | | | | LNPGAYIYCWSYDYWGCGTLVTVSS | |
| | | CTIGGSLSRSSQGTLVTVSS | | | | GGC | |
| T028100075 | 176 | DVQLVESGGGVVQPGGSL | 106 | GGGGSGGG | 163 | EVQLVESGGGLVQAGGSLSISCAASG | 175 |
| | | RLSCAASGFTFRSFGMSWV | | GSGGGGS | | GSLSNYVLGWFRQAPGKEREFVAAI | |
| | | RQAPGKGPEWVSSISGSGS | | | | NWRGDITIGPPNVEGRFTISRDNAKN | |
| | | DTLYADSVKGRFTISRDNSK | | | | TGYLQMNSLAPDDTAVYYCGAGTPL | |
| | | NTLYLQMNSLRPEDTALYY | | | | NPGAYIYDWSYDYWGRGTLVTVSSG | |
| | | CTIGGSLSRSSQGTLVTVSS | | | | GC | |

Examples of polypeptides comprising a half-life extending moiety which is an albumin-binding ISVD and a protein-based carrier building block which is derived from the small globular protein CKS are depicted in SEQ ID NO.: 123-127 and 170-171, see Table 12 below.

TABLE 12
Examples of polypeptides comprising a half-life extending moiety which is an
albumin-binding ISVD and a protein-based carrier building block which is derived from the
small globular protein CKS (″ID″ refers to the SEQ ID NO as used herein)

| Polypeptide | ID | albumin-binding ISVD | ID | Linker | ID | CKS-derived building block | ID |
|---|---|---|---|---|---|---|---|
| ALB- | 123 | DVQLVESGGGVVQPGGSL | 106 | GGGGSGGG | 163 | SHKQIYYSDCYDCEEFEYRHVMLPKDI | 99 |
| 3C_hCKS1_ | | RLSCAASGFTFRSFGMSWV | | | | AKLVPKTHLMSESEWRNLGVQQSQ | |
| c1 | | RQAPGKGPEWVSSISGSGS | | GSGGGGS | | GWVHYMIHEPEPHILLFRRPLPKKPK | |
| | | DTLYADSVKGRFTISRDNSK | | | | C | |
| | | NTLYLQMNSLRPEDTALYY | | | | | |
| | | CTIGGSLSRSSQGTLVTVSS | | | | | |
| ALB- | 124 | DVQLVESGGGVVQPGGSL | 106 | GGGGSGGG | 163 | SHKQIYYSDCYCDEEFEYRHVCLPKDI | 100 |
| 3C_hCKS1_ | | RLSCAASGFTFRSFGMSWV | | GSGGGGS | | AKLVPKTHLMSESEWRNLGVQQSCG | |
| c2 | | RQAPGKGPEWVSSISGSGS | | | | WVHYMIHEPEPHILLFRRPLPKKPKK | |
| | | DTLYADSVKGRFTISRDNSK | | | | | |
| | | NTLYLQMNSLRPEDTALYY | | | | | |
| | | CTIGGSLSRSSQGTLVTVSS | | | | | |
| ALB- | 125 | IDVQLVESGGGVVQPGGSL | 106 | GGGGSGGG | 163 | SHKQIYYSDKCDDEEFEYRHVMLPKD | 101 |
| 3C_hCKS1_ | | RLSCAASGFTFRSFGMSWV | | GSGGGGS | | IAKLVPKTHLMSESEWRNLGVQQSC | |
| c3 | | RQAPGKGPEWVSSISGSGS | | | | GWVHYMIHEPEPHILLFRRPLPKKPK | |
| | | DTLYADSVKGRFTISRDNSK | | | | C | |
| | | NTLYLQMNSLRPEDTALYY | | | | | |
| | | CTIGGSLSRSSQGTLVTVSS | | | | | |
| ALB- | 126 | DVQLVESGGGVVQPGGSL | 106 | GGGGSGGG | 163 | SHKQIYYSDKYDCEEFEYRHVMLPKDI | 102 |
| 3C_hCKS1_ | | RLSCAASGFTFRSFGMSWV | | GSGGGGS | | AKLVPCTHLMSESEWRNLGVQQSQ | |
| w1 | | RQAPGKGPEWVSSISGSGS | | | | GWVHYMIHEPEPHILLFRRPLPKKPK | |
| | | DTLYADSVKGRFTISRDNSK | | | | C | |
| | | NTLYLQMNSLRPEDTALYY | | | | | |
| | | CTIGGSLSRSSQGTLVTVSS | | | | | |
| ALB- | 127 | DVQLVESGGGVVQPGGSL | 106 | GGGGSGGG | 163 | SHKQIYYSDKCDDEEFEYRHVMLPKD | 103 |
| 3C_hCKS1_ | | RLSCAASGFTFRSFGMSWV | | GSGGGGS | | IAKLVPCTHLMSESEWRNLGVQQSC | |
| w2 | | RQAPGKGPEWVSSISGSGS | | | | GWVHYMIHEPEPHILLFRRPLPKKPK | |
| | | DTLYADSVKGRFTISRDNSK | | | | C | |
| | | NTLYLQMNSLRPEDTALYY | | | | | |
| | | CTIGGSLSRSSQGTLVTVSS | | | | | |
| ALB-6C_ | 170 | DVQLVESGGGVVQPGGSL | 106 | GGGGSGGG | 163 | SHKQIYYSCKYDCEEFEYRHVCLPKDI | 104 |
| hCKS1 | | RLSCAASGFTFRSFGMSWV | | GSGGGGS | | AKLVPCTHLMSESEWRNLGVQQSCG | |
| | | RQAPGKGPEWVSSISGSGS | | | | WVHYMIHEPEPHILLFRRPLPKKPKC | |
| | | DTLYADSVKGRFTISRDNSK | | | | | |
| | | NTLYLQMNSLRPEDTALYY | | | | | |
| | | CTIGGSLSRSSQGTLVTVSS | | | | | |
| ALB- | 171 | DVQLVESGGGVVQPGGSL | 106 | GGGGSGGG | 163 | SHKQIYYSCKYDCEEFEYRHVMLPKDI | 105 |
| 6C_hCKS1_ | | RLSCAASGFTFRSFGMSWV | | GSGGGGS | | AKLVPCTHLMSESEWRNLGVQQSCG | |
| tr | | RQAPGKGPEWVSSISGSGS | | | | WVHYCIHEPEPHILLFRRPLC | |
| | | DTLYADSVKGRFTISRDNSK | | | | | |
| | | NTLYLQMNSLRPEDTALYY | | | | | |
| | | CTIGGSLSRSSQGTLVTVSS | | | | | |

Examples of polypeptides comprising a half-life extending moiety which is an albumin-binding ISVD and a protein-based carrier building block which is derived from DARPins are depicted in SEQ ID NO.: 172-174 and 200, see Table 13 below.

TABLE 13
Examples of polypeptides comprising a half-life extending moiety which is an
albumin-binding ISVD and a protein-based carrier building block which is a DARPin-based
building block (″ID″ refers to the SEQ ID NO as used herein)

| Polypeptide | ID | albumin-binding ISVD | ID | Linker | ID | DARPin-based building block | ID |
|---|---|---|---|---|---|---|---|
| ALB- | 172 | DVQLVESGGGVVQPGGSL | 106 | GGGGSGGG | 163 | DLGKKLLEAARAGQDDEVRILMANG | 96 |
| 3C_K27m_c1 | | RLSCAASGFTFRSFGMSWV | | GSGGGGS | | ADVNAHDTFGFTPLHLAALYGHLEIV | |
| | | RQAPGKGPEWVSSISGSGS | | | | EVLLKNGADVNADDSYGATPLHLAA | |
| | | DTLYADSVKGRFTISRDNSK | | | | MRGHLEIVEVLLKYGADVNAADEEG | |
| | | NTLYLQMNSLRPEDTALYY | | | | ATPLHLAAKAGHLEIVEVLLKNGADV | |
| | | CTIGGSLSRSSQGTLVTVSS | | | | NAQDKFGKTAFDISICNGNECLAEILQ | |
| | | | | | | KC | |
| ALB- | 173 | DVQLVESGGGVVQPGGSL | 106 | GGGGSGGG | 163 | DLGKKLLEAARAGQDDEVRILMANG | 97 |
| 3C_K27m_w1 | | RLSCAASGFTFRSFGMSWV | | GSGGGGS | | ADVNAHDTFGFTPLHLAALYGHLEIV | |
| | | RQAPGKGPEWVSSISGSGS | | | | EVLLKNGADVNADDSYGATPLHLAA | |
| | | DTLYADSVKGRFTISRDNSK | | | | MRGHLEIVCVLLKYGADVCAADEEG | |
| | | NTLYLQMNSLRPEDTALYY | | | | ATPLHLAAKAGHLEIVEVLLKNGADV | |
| | | CTIGGSLSRSSQGTLVTVSS | | | | NAQDKFGKTAFDISIDNGNEDLAEIL | |
| | | | | | | QKC | |
| ALB-5C_K27m | 174 | DVQLVESGGGVVQPGGSL | 106 | GGGGSGGG | 163 | DLGKKLLEAARAGQDDEVRILMANG | 98 |
| | | RLSCAASGFTFRSFGMSWV | | GSGGGGS | | ADVNAHDTFGFTPLHLAALYGHLEIV | |
| | | RQAPGKGPEWVSSISGSGS | | | | EVLLKNGADVNADDSYGATPLHLAA | |
| | | DTLYADSVKGRFTISRDNSK | | | | MRGHLEIVCVLLKYGADVCAADEEG | |
| | | NTLYLQMNSLRPEDTALYY | | | | ATPLHLAAKAGHLEIVEVLLKNGADV | |
| | | CTIGGSLSRSSQGTLVTVSS | | | | NAQDKFGKTAFDISICNGNECLAEILQ | |
| | | | | | | KC | |
| ALB-1C_K27m | 200 | DVQLVESGGGVVQPGGSL | 106 | GGGGSGGG | 163 | DLGKKLLEAARAGQDDEVRILMANG | 199 |
| | | RLSCAASGFTFRSFGMSWV | | GSGGGGS | | ADVNAHDTFGFTPLHLAALYGHLEIV | |
| | | RQAPGKGPEWVSSISGSGS | | | | EVLLKNGADVNADDSYGATPLHLAA | |
| | | DTLYADSVKGRFTISRDNSK | | | | MRGHLEIVEVLLKYGADVNAADEEG | |
| | | NTLYLQMNSLRPEDTALYY | | | | ATPLHLAAKAGHLEIVEVLLKNGADV | |
| | | CTIGGSLSRSSQGTLVTVSS | | | | NAQDKFGKTAFDISIDNGNEDLAEIL | |
| | | | | | | QKC | |

### Targeting Moieties

As described above, the protein-based carrier building block may have attached or conjugated, via its one or more conjugation sites or attachment points, one or more groups, residues, moieties or binding units, optionally attached via one or more linkers, in which said one or more other groups, residues, moieties or binding units target the molecule of the present technology to target molecules on cells, organs or tissues (“targeting moiety”). For instance, the molecule of the present technology may comprise one, two, three, four, five, six, seven, eight, nine, ten or more targeting moieties attached or conjugated to the at least one protein-based carrier building block.

A targeting moiety, as defined herein, is any group, residue, moiety, or binding unit which is capable of being directed through its binding to a target. An amino acid sequence (such as an ISVD, an antibody, antigen-binding domains or fragments such as VHH domains or VH/VL domains, or generally an antigen binding protein or polypeptide or a fragment thereof) that “(specifically) binds”, that “can (specifically) bind to”, that “has affinity for” and/or that “has specificity for” a specific antigenic determinant, epitope, antigen or protein, or for a specific non-protein molecule, such as nucleic acids (such as DNA or RNA) or glycans (or for at least one part, fragment or epitope thereof) is said to be “against” or “directed against” said antigenic determinant, epitope, antigen, protein or non-protein molecule. Specific binding of an antigen-binding protein to an antigen or antigenic determinant can be determined in any suitable manner known per se, including, for example, Scatchard analysis and/or competitive binding assays, such as radio-immunoassays (RIA), enzyme immunoassays (EIA) and sandwich competition assays, and the different variants thereof known per se in the art; as well as the other techniques mentioned herein.

For instance, the molecule of the present technology may comprise a targeting moiety such as blood brain barrier (BBB) shuttling moieties, HM-3 integrin antagonists, cell-penetrating peptides (CPPs), short linear motifs (SLIMs) such as retinoblastoma-binding LxCxE motif or nuclear localization signals, folic acid, distearoyl, cholesterol, targeting nucleic acids and the like.

Non-limiting examples of targeting moieties which may be present in the molecule of the present technology, e.g., attached to the at least one protein-based carrier building block through the at least one attachment point or conjugation sites are the following:
Human serum albumin (HSA)-binding molecules, as described, e.g., in WO 2006/122787, WO 2011/095545, WO 2012/175400, WO 2017/085172 or WO 2018/104444, the content of which is herewith incorporated by reference. Any of the HSA-binding molecules described in these documents may be incorporated in the molecule of the present technology, e.g., by attaching it (directly or via a linker, as described herein) to the protein-based carrier building block through the one or more attachment points or conjugation sites.
Aggrecan-binding molecules, as described, e.g., in WO 2018/220225, the content of which is incorporated herewith by reference. Any of the aggrecan-binding molecules described in WO 2018/220225 may be incorporated in the molecule of the present technology, e.g., by attaching it (directly or via a linker, as described herein) to the protein-based carrier building block through the one or more attachment points or conjugation sites.
Glypican-3 (GPC3)-binding molecules, as described, e.g., in WO 2022/129560 the content of which is incorporated herewith by reference. Any of the GPC3-binding molecules described in WO 2022/129560 may be incorporated in the molecule of the present technology, e.g., by attaching it (directly or via a linker, as described herein) to the protein-based carrier building block through the one or more attachment points or conjugation sites.
T-cell receptor (TCR)-binding molecules, as described, e.g., in WO 2016/180969, WO 2018/091606 or WO 2022/129637. Any of the TCR-binding molecules described in these documents may be incorporated in the molecule of the present technology, e.g., by attaching it (directly or via a linker, as described herein) to the protein-based carrier building block through the one or more attachment points or conjugation sites.
Neonatal Fc receptor (FcRn)-binding molecules, as described, e.g., in WO 2008/074867 or WO 2009/080764. Any of the FcRn-binding molecules described in these documents may be incorporated in the molecule of the present technology, e.g., by attaching it (directly or via a linker, as described herein) to the protein-based carrier building block through the one or more attachment points or conjugation sites.
Polymeric immunoglobulin receptor (pIgR)-binding molecules, as described, e.g., also in WO 2008/074867 or WO 2009/080764. Any of the plgR-binding molecules described in these documents may be incorporated in the molecule of the present technology, e.g., by attaching it (directly or via a linker, as described herein) to the protein-based carrier building block through the one or more attachment points or conjugation sites.
Interleukin-6 receptor (IL-6R)-binding molecules, as described, e.g., in WO 2010/115995 or WO 2010/115998. Any of the anti-IL-6R sequences described in WO 2010/115995 or WO 2010/115998 may be attached to or conjugated to the attachment point(s) or conjugation site(s) of the protein-based building block of the present technology.
Vascular endothelial growth factor receptor 1 (VEGF-R1, also called Flt-I)-binding molecules, as described, e.g., in WO 2008/142165. Any of the VEGF-R1 sequences described in WO 2008/142165 may be attached to or conjugated (directly or via a linker, as described herein) to the attachment point(s) or conjugation site(s) of the protein-based building block of the present technology.
Platelet derived growth factor receptor beta (PDGF-RB)-binding molecules, as described, e.g., in WO 2008/142165. Any of the PDGF-RB-binding sequences described in WO 2008/142165 may be attached or conjugated (directly or via a linker, as described herein) to the attachment point(s) or conjugation site(s) of the protein-based building block of the present technology.
Fibroblast growth factor receptor 4 (FGF-R4)-binding molecules, as described, e.g., in WO 2008/142165. Any of the FGF-R4-binding sequences described in WO 2008/142165 may be attached to or conjugated (directly or via a linker, as described herein) to the attachment point(s) or conjugation site(s) of the protein-based building block of the present technology.
Epidermal growth factor receptor (EGFR)-binding molecules, as described, e.g., in WO 2005/044858, WO 2007/042289 or WO 2016/097313. Any of the EGFR-binding sequences described in WO 2005/044858, WO 2007/042289 or WO 2016/097313 may be attached or conjugated (directly or via a linker, as described herein) to the attachment point(s) or conjugation site(s) of the protein-based building block of the present technology.

In addition, EGFR-binding oligopeptide GE11 (YHWYGYTPQNVI, SEQ ID NO.: 212, Mw (Molecular weight) 1540 g/mol, IP 7.67) may be attached or conjugated (directly or via a linker, as described herein) to the attachment point(s) or conjugation site(s) of the protein-based building block of the present technology. GE11 is a dodecapeptide with excellent EGFR affinity. It actively binds, similar to human EGF or anti-EGFR monoclonal antibody cetuximab, the surface of EGFR-positive tumor cells. GE11 is a potentially safe and efficient targeting moiety for selective drug delivery systems mediated through EGFR (cf. Li Z. et al., “Identification and characterization of a novel peptide ligand of epidermal growth factor receptor for targeted delivery of therapeutics”, FASEB J., 2005, 19 (14): 1978-85). Hence, GE11 can be used to bind to and internalize in EGFR+tumor cells and can be synthesized by solid-phase peptide synthesis. For more details, see, e.g., Pola R. et al., “Targeted polymer-based probes for fluorescence guided visualization and potential surgery of EGFR-positive head-and-neck tumors”, Pharmaceutics, 2020, 12 (1): 31 or Hailing T. et al., “Challenges for the application of EGFR-targeting peptide GE11 in tumor diagnosis and treatment”, J Control Release, 2022, 349:592-605.

Hence, the present technology provides molecules as defined herein which comprises at least one protein-based building block with at least one GE11 peptide attached to at least one attachment point or conjugation site.

For instance, the present technology provides one molecule as described herein which comprises at least one ISVD-based building block, as defined herein, with at least one GE11 peptide attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, the molecule comprising at least one such ISVD-derived protein-based building block and at least one GE11 peptide attached to it through the at least one conjugation site or attachment point, does not specifically bind to any non-protein molecule and/or does not specifically bind to any non-human protein to which the ISVD precursor specifically binds.

For instance, the present technology provides one molecule as described herein which comprises at least one DARPin-based building block, as defined herein, with at least one GE11 peptide attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one GE11 peptide attached to at least one attachment point or conjugation site.

For instance, the present technology provides one molecule as described herein which comprises at least one building block based on a small globular protein, such as CKS1, as defined herein, with at least one GE11 peptide attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one DARPin-based building block selected from SEQ ID NO.: 199, 97 and/or 98, preferably SEQ ID NO.: 97 or 98, and at least one, preferably more than one, such as two, or, preferably, three GE11 peptides conjugated to the attachment points or conjugation sited of the DARPin-based building blocks. Preferably, the molecule further comprises a HLE moiety, such as an albumin-binding ISVD, e.g., SEQ ID NO.: 106. In one embodiment, the at least one GE11 peptide is conjugated to the DARPin-based building block comprised in molecule ALB-1C_K27m (SEQ ID NO.: 200), ALB-3C_K27m_w1 (SEQ ID NO.: 173) and/or ALB-5C_K27m (SEQ ID NO.: 174), preferably ALB-3C_K27m_w1 and/or ALB-5C_K27m.

In one embodiment, the molecule of the present technology comprises at least one ISVD-based building block selected from SEQ ID NO.: 80, 81 or 175, preferably SEQ ID NO.: 80 or 81, and at least one, preferably more than one, such as two, or, preferably, three GE11 peptides conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one GE11 peptide is conjugated to the ISVD-based building block comprised in molecule T028100069 (SEQ ID NO.: 107), T028100070 (SEQ ID NO.: 108) and/or T028100075 (SEQ ID NO.: 176), preferably T028100069 and/or T028100070.

As described above, there are preferably more than one GE11 peptides conjugated to the ISVD- and/or DARPin-derived building blocks, preferably 3 GE11 peptides per ISVD-based building block.
Human epidermal growth factor receptor 2 (HER-2 or receptor tyrosine-protein kinase erbB-2)-binding molecules, as described, e.g., in WO 2009/068625. Any of the HER-2-binding sequences described in WO 2009/068625 may be attached or conjugated (directly or via a linker, as described herein) to the attachment point(s) or conjugation site(s) of the protein-based building block of the present technology.
Von Willebrand factor (vWF)-binding molecules, as described in, e.g., WO 2006/122825. Any of the vWF-2-binding sequences described in WO 2009/068625 may be attached or conjugated (directly or via a linker, as described herein) to the attachment point(s) or conjugation site(s) of the protein-based building block of the present technology.
Blood-brain barrier (BBB) delivery molecules, such as peptides (see, e.g., Sánchez-Navarro and Giralt, “Peptide shuttles for blood-brain barrier drug delivery”, Pharmaceutics 2022, 14, 1874) or other carrier systems (see, e.g., Mahringer et al., “Crossing the blood-brain barrier: A review on drug delivery strategies using colloidal carrier systems”, Neurochem Int., 2021, 147:105017).
HM-3 integrin antagonist, see, e.g., Li T., et al., “Albumin Fusion at the N-Terminus or C-Terminus of HM-3 Leads to Improved Pharmacokinetics and Bioactivities”, Biomedicines, 2021, 9 (9): 1084.
Cell-penetrating peptides, see, e.g., Xie et al., “Cell-penetrating peptides in diagnosis and treatment of human diseases: from preclinical research to clinical application”, Front. Pharmacol., 2020, 11:697.
Short linear motifs (SLIMs), such as retinoblastoma-binding LxCxE motif or nuclear localization signals.
Targeting nucleic acids, see, e.g., Kulkarni, J. A., Witzigmann, D., Thomson, S. B. et al., “The current landscape of nucleic acid therapeutics”, Nat. Nanotechnol., 2021, 16, 630-643.
Folic acid, distearoyl, cholesterol, and the like.

### Therapeutic Moieties or Precursors Therefrom

As described above, the protein-based carrier building block of the present technology may have attached or conjugated, via its one or more conjugation sites or attachment points, one or more other groups, residues, moieties or binding units, optionally linked via one or more peptidic linkers, in which said one or more other groups, residues, moieties or binding units are capable of exerting a therapeutic activity in the animal or human body (“therapeutic moiety or precursor therefrom”). For instance, the molecule of the present technology may comprise one, two, three, four, five, six, seven, eight, nine, ten or more therapeutic moieties or precursors therefrom attached or conjugated to the at least one protein-based carrier building block.

A therapeutic moiety, as defined herein, is any group, residue, moiety, or binding unit which is capable of exerting a therapeutic activity in the animal and/or human body. The therapeutic moiety may also be in the form of a precursor, which then gets activated to exert its therapeutic activity. For instance, a therapeutic moiety according to the present technology may be any therapeutic agent such as a drug, protein, peptide, gene, compound or any other pharmaceutically active ingredient which may be used for the treatment and/or prevention of a certain disease condition. For instance, a therapeutic moiety may be a therapeutic antibody, or a therapeutic ISVD.

Non-limiting examples of therapeutic moieties which may be present in the molecule of the present technology, e.g., attached to the at least one protein-based carrier building block through the at least one attachment point or conjugation sites are the following:
Epidermal growth factor receptor (EGFR)-binding molecules, as described, e.g., in WO 2005/044858, WO 2007/042289 or WO 2016/097313. Any of the EGFR-binding sequences described in WO 2005/044858, WO 2007/042289 or WO 2016/097313 may be attached or conjugated (directly or via a linker, as described herein) to the attachment point(s) or conjugation site(s) of the protein-based building block of the present technology.

In addition, EGFR-binding oligopeptide GE11 (YHWYGYTPQNVI, SEQ ID NO.: 212, Mw (Molecular weight) 1540 g/mol, IP 7.67) may be attached or conjugated (directly or via a linker, as described herein) to the attachment point(s) or conjugation site(s) of the protein-based building block of the present technology. GE11 is a dodecapeptide with excellent EGFR affinity. It actively binds, similar to human EGF or anti-EGFR monoclonal antibody cetuximab, the surface of EGFR-positive tumor cells. GE11 is a potentially safe and efficient targeting moiety for selective drug delivery systems mediated through EGFR (cf. Li Z. et al., “Identification and characterization of a novel peptide ligand of epidermal growth factor receptor for targeted delivery of therapeutics”, FASEB J., 2005, 19 (14): 1978-85). Hence, GE11 can be used to bind to and internalize in EGFR+tumor cells and can be synthesized by solid-phase peptide synthesis. For more details, see, e.g., Pola R. et al., “Targeted polymer-based probes for fluorescence guided visualization and potential surgery of EGFR-positive head-and-neck tumors”, Pharmaceutics, 2020, 12 (1): 31 or Hailing T. et al., “Challenges for the application of EGFR-targeting peptide GE11 in tumor diagnosis and treatment”, J Control Release, 2022, 349:592-605.

Hence, the present technology provides molecules as defined herein which comprises at least one protein-based building block with at least one GE11 peptide attached to at least one attachment point or conjugation site.

For instance, the present technology provides one molecule as described herein which comprises at least one ISVD-based building block, as defined herein, with at least one GE11 peptide attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one GE11 peptide, through the at least one conjugation site or attachment point, comprised in the molecule of the present technology, does not specifically bind to any target, such as protein and/or non-protein molecules, including biomolecules, to which the ISVD precursor specifically binds.

For instance, the present technology provides one molecule as described herein which comprises at least one DARPin-based building block, as defined herein, with at least one GE11 peptide attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one GE11 peptide attached to at least one attachment point or conjugation site.

For instance, the present technology provides one molecule as described herein which comprises at least one building block based on a small globular protein, such as CKS1, as defined herein, with at least one GE11 peptide attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one DARPin-based building block selected from SEQ ID NO.: 199, 97 and/or 98, preferably SEQ ID NO.: 97 or 98, and at least one, preferably more than one, such as two, or three or, preferably, five GE11 peptides conjugated to the attachment points or conjugation sited of the DARPin-based building blocks. Preferably, the molecule further comprises a HLE moiety, such as an albumin-binding ISVD, e.g., SEQ ID NO.: 106. In one embodiment, the at least one GE11 peptide is conjugated to the DARPin-based building block comprised in molecule ALB-1C_K27m (SEQ ID NO.: 200), ALB-3C_K27m_w1 (SEQ ID NO.: 173) and/or ALB-5C_K27m (SEQ ID NO.: 174), preferably ALB-3C_K27m_w1 and/or ALB-5C_K27m.

In one embodiment, the molecule of the present technology comprises at least one ISVD-based building block selected from SEQ ID NO.: 80, 81 or 175, preferably SEQ ID NO.: 80 or 81, and at least one, preferably more than one, such as two, three or, preferably, five GE11 peptides conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one GE11 peptide is conjugated to the ISVD-based building block comprised in molecule T028100069 (SEQ ID NO.: 107), T028100070 (SEQ ID NO.: 108) and/or T028100075 (SEQ ID NO.: 176), preferably T028100069 and/or T028100070.

As described above, there are preferably more than one GE11 peptides conjugated to the ISVD- and/or DARPin-derived building blocks, preferably five GE11 peptides per ISVD-based building block.
Von Willebrand factor (vWF)-binding molecules, as described in, e.g., WO 2006/122825. Any of the vWF-binding sequences described in WO 2006/122825 may be attached or conjugated (directly or via a linker, as described herein) to the attachment point(s) or conjugation site(s) of the protein-based building block of the present technology.
Human epidermal growth factor receptor 2 (HER-2 or receptor tyrosine-protein kinase erbB-2)-binding molecules, as described, e.g., in WO 2009/068625. Any of the HER-2-binding sequences described in WO 2009/068625 may be attached or conjugated (directly or via a linker, as described herein) to the attachment point(s) or conjugation site(s) of the protein-based building block of the present technology.
Interleukin-6 receptor (IL-6R)-binding molecules, as described, e.g., in WO 2010/115995 or WO 2010/115998. Any of the anti-IL-6R sequences described in WO 2010/115995 or WO 2010/115998 may be attached or conjugated (directly or via a linker, as described herein) to the attachment point(s) or conjugation site(s) of the protein-based building block of the present technology.
Neonatal Fc receptor (FcRn)-binding molecules, as described, e.g., in WO 2008/074867 or WO 2009/080764. Any of the FcRn-binding sequences described in WO 2008/074867 or WO 2009/080764 may be attached or conjugated (directly or via a linker, as described herein) to the attachment point(s) or conjugation site(s) of the protein-based building block of the present technology.
Polymeric immunoglobulin receptor (pIgR)-binding molecules, as described, e.g., also in WO 2008/074867 or WO 2009/080764. Any of the pIgR-binding sequences described in WO 2008/074867 or WO 2009/080764 may be attached or conjugated (directly or via a linker, as described herein) to the attachment point(s) or conjugation site(s) of the protein-based building block of the present technology.
Vascular endothelial growth factor receptor 1 (VEGF-R1, also called Flt-I)-binding molecules, as described, e.g., in WO 2008/142165. Any of the VEGF-R1 sequences described in WO 2008/142165 may be attached or conjugated (directly or via a linker, as described herein) to the attachment point(s) or conjugation site(s) of the protein-based building block of the present technology.
Platelet derived growth factor receptor beta (PDGF-RB)-binding molecules, as described, e.g., in WO 2008/142165. Any of the PDGF-RB-binding sequences described in WO 2008/142165 may be attached or conjugated (directly or via a linker, as described herein) to the attachment point(s) or conjugation site(s) of the protein-based building block of the present technology.
Fibroblast growth factor receptor 4 (FGF-R4)-binding molecules, as described, e.g., in WO 2008/142165. Any of the FGF-R4-binding sequences described in WO 2008/142165 may be attached or conjugated (directly or via a linker, as described herein) to the attachment point(s) or conjugation site(s) of the protein-based building block of the present technology.
Tumor necrosis factor-α (TNF-α)-binding molecules, as described, e.g., in WO 2006/122786, WO 2015/173325, WO 2017/081320, WO 2021/110816, WO 2021/110817, or WO 2022/129572. Any of the TNF-α-binding sequences described in any of WO 2006/122786, WO 2015/173325, WO 2017/081320, WO 2021/110816, WO 2021/110817 or WO 2022/129572 may be attached or conjugated (directly or via a linker, as described herein) to the attachment point(s) or conjugation site(s) of the protein-based building block of the present technology.
Insulin-like growth factor 1 receptor (IGF-IR)-binding molecules, as described in, e.g., WO 2007/042289. Any of the IGF-IR-binding sequences described in WO 2007/042289 may be attached or conjugated (directly or via a linker, as described herein) to the attachment point(s) or conjugation site(s) of the protein-based building block of the present technology.
Vascular endothelial growth factor (VEGF)-binding molecules, as described, e.g., in WO 2008/101985. Any of the VEGF-binding sequences described in WO 2008/101985 may be attached or conjugated (directly or via a linker, as described herein) to the attachment point(s) or conjugation site(s) of the protein-based building block of the present technology.
Receptor activator of nuclear factor kappa-β ligand (RANK-L)-binding molecules, as described, e.g., in WO 2008/142164 or WO 2015/173325. Any of the RANK-L-binding sequences described in any of WO2008/142164 or WO 2015/173325 may be attached or conjugated (directly or via a linker, as described herein) to the attachment point(s) or conjugation site(s) of the protein-based building block of the present technology.
Interleukin 23 (IL-23)-binding molecules, as described, e.g., in WO 2009/068627, WO 2011/135026, WO 2011/161263, WO 2015/173325, WO 2017/072299 or WO 2021/110816. Any of the IL-23-binding sequences described in any of WO 2009/068627, WO 2011/135026, WO 2011/161263, WO 2015/173325, WO 2017/072299 or WO 2021/110816 may be attached or conjugated (directly or via a linker, as described herein) to the attachment point(s) or conjugation site(s) of the protein-based building block of the present technology.
Respiratory syncytial virus (RSV) fusion (F) protein binding molecules, as described, e.g., in WO 2009/147248 or WO 2010/139808. Any of the RSV-binding sequences described in any of WO 2009/147248 or WO 2010/139808 may be attached or conjugated (directly or via a linker, as described herein) to the attachment point(s) or conjugation site(s) of the protein-based building block of the present technology.
Influenza H5N1 hemagglutinin (HA)-binding molecules, as described, e.g., in WO 2009/147248. Any of the Influenza H5N1 hemagglutinin (HA)-binding sequences described in WO 2009/147248 may be attached or conjugated (directly or via a linker, as described herein) to the attachment point(s) or conjugation site(s) of the protein-based building block of the present technology.
Rabies virus G-protein-binding molecules, as described, e.g., in WO 2009/147248. Any of the Rabies virus G-protein-binding sequences described in WO 2009/147248 may be attached or conjugated (directly or via a linker, as described herein) to the attachment point(s) or conjugation site(s) of the protein-based building block of the present technology.
CXC chemokine receptor type 4 (CXCR4)-binding molecules, as described, e.g., in WO 2009/138519, WO 2011/083141, WO 2011/161266, WO 2015/044386, WO 2015/173325 or WO 2016/156570. Any of the CXCR4-binding sequences described in any of WO 2009/138519, WO 2011/083141, WO 2011/161266, WO 2015/044386, WO 2015/173325 or WO 2016/156570 may be attached or conjugated (directly or via a linker, as described herein) to the attachment point(s) or conjugation site(s) of the protein-based building block of the present technology.
CXC chemokine receptor type 7 (CXCR7)-binding molecules, as described, e.g., in WO 2009/138519, WO 2011/117423, WO 2012/130874 or WO 2015/173325. Any of the CXCR7-binding sequences described in any of WO 2009/138519, WO 2011/117423, WO 2012/130874 or WO 2015/173325 may be attached or conjugated (directly or via a linker, as described herein) to the attachment point(s) or conjugation site(s) of the protein-based building block of the present technology.
Sclerostin-binding molecules, as described, e.g., in WO 2010/130830. Any of the sclerostin-binding sequences described in WO 2010/130830 may be attached or conjugated (directly or via a linker, as described herein) to the attachment point(s) or conjugation site(s) of the protein-based building block of the present technology.
Dickkopf-1 (Dkk-1)-binding molecules, as described, e.g., in WO 2010/130832. Any of the Dkk-1-binding sequences described in WO 2010/130832 may be attached or conjugated (directly or via a linker, as described herein) to the attachment point(s) or conjugation site(s) of the protein-based building block of the present technology.
HER-3-binding molecules, as described, e.g., in WO 2011/144749 or WO 2015/173325. Any of the HER3-binding sequences described in any of WO 2011/144749 or WO 2015/173325 may be attached or conjugated (directly or via a linker, as described herein) to the attachment point(s) or conjugation site(s) of the protein-based building block of the present technology.
c-Met-binding molecules, as described, e.g., in WO 2012/042026, WO 2013/045707 or WO 2015/173325. Any of the c-Met-binding sequences described in any of WO 2012/042026, WO 2013/045707 or WO 2015/173325 may be attached or conjugated (directly or via a linker, as described herein) to the attachment point(s) or conjugation site(s) of the protein-based building block of the present technology.
Amyloid beta (Aβ or A-beta)-binding molecules, as described, e.g., in WO 2011/107507 or WO 2015/173325. Any of the A-beta-binding sequences described in any of WO 2011/107507 or WO 2015/173325 may be attached or conjugated (directly or via a linker, as described herein) to the attachment point(s) or conjugation site(s) of the protein-based building block of the present technology.
CXC chemokine receptor type 2 (CXCR2)-binding molecules, as described, e.g., in WO 2012/062713 or WO 2013/168108. Any of the CXCR2-binding sequences described in any of WO 2012/062713 or W O2013/168108 may be attached or conjugated (directly or via a linker, as described herein) to the attachment point(s) or conjugation site(s) of the protein-based building block of the present technology.
Immunoglobulin E (IgE)-binding molecules, as described, e.g., in WO 2012/175740, WO 2014/087010 or WO 2015/173325. Any of the IgE-binding sequences described in any of WO 2012/175740, WO 2014/087010 or WO 2015/173325 may be attached or conjugated (directly or via a linker, as described herein) to the attachment point(s) or conjugation site(s) of the protein-based building block of the present technology.
Interleukin-17 (IL-17) A, IL-17F and/or IL-17A/F-binding molecules, as described, e.g., in WO 2012/156219. Any of the IL-17A, IL-17F and/or IL-17A/F-binding sequences described in WO 2012/156219 may be attached or conjugated (directly or via a linker, as described herein) to the attachment point(s) or conjugation site(s) of the protein-based building block of the present technology.
Hepatocyte growth factor (HGF)-binding molecules, as described, e.g., in WO 2013/110531. Any of the HGF-binding sequences described in WO 2013/110531 may be attached or conjugated (directly or via a linker, as described herein) to the attachment point(s) or conjugation site(s) of the protein-based building block of the present technology.
Pseudomonas aeruginosa PcrV-binding molecules, as described, e.g., in WO 2013/128031. Any of the Pseudomonas aeruginosa PcrV-binding sequences described in WO 2013/110531 may be attached or conjugated (directly or via a linker, as described herein) to the attachment point(s) or conjugation site(s) of the protein-based building block of the present technology.
P2X7-binding molecules, as described, e.g., in WO 2013/178783. Any of the P2X7-binding sequences described in WO 2013/178783 may be attached or conjugated (directly or via a linker, as described herein) to the attachment point(s) or conjugation site(s) of the protein-based building block of the present technology.
Interleukin-3 receptor (CD123)-binding molecules, as described, e.g., in WO 2015/044386 or WO 2018/091606. Any of the CD123-binding sequences described in any of WO 2015/044386 or WO 2018/091606 may be attached or conjugated (directly or via a linker, as described herein) to the attachment point(s) or conjugation site(s) of the protein-based building block of the present technology.
Interleukin-3 receptor alpha (IL-3Ra)-binding molecules, as described, e.g., in WO 2015/044386. Any of the IL-3Ra-binding sequences described in WO 2015/044386 may be attached or conjugated (directly or via a linker, as described herein) to the attachment point(s) or conjugation site(s) of the protein-based building block of the present technology.
Potassium voltage-gated channel, shaker-related subfamily, member 3 (Kᵥ1.3)-binding molecules, as described, e.g., in WO 2015/193452 or WO 2015/173325. Any of the Kᵥ1.3-binding molecules described in WO 2015/193452 or WO 2015/173325 may be attached or conjugated (directly or via a linker, as described herein) to the attachment point(s) or conjugation site(s) of the protein-based building block of the present technology.
OX40L-binding molecules, as described, e.g., in WO 2011/073180, WO 2015/173325, WO 2021/110817 or WO 2022/063984. Any of the OX40L-binding molecules described in any one of WO 2011/073180, WO 2015/173325, WO 2021/110817 or WO 2022/063984 may be attached or conjugated (directly or via a linker, as described herein) to the attachment point(s) or conjugation site(s) of the protein-based building block of the present technology.
CD40L-binding molecules, as described, e.g., in WO 2017/089618. Any of the CD40L-binding molecules described in WO 2017/089618 may be attached or conjugated (directly or via a linker, as described herein) to the attachment point(s) or conjugation site(s) of the protein-based building block of the present technology.
T-cell receptor (TCR)-binding molecules, as described, e.g., in WO 2016/180969, WO 2018/091606 or WO 2022/129637. Any of the TCR-binding molecules described in any one of WO 2016/180969, WO 2018/091606 or WO 2022/129637 may be attached or conjugated (directly or via a linker, as described herein) to the attachment point(s) or conjugation site(s) of the protein-based building block of the present technology.
CD-4-binding molecules, as described, e.g., in WO 2016/156570. Any of the CD-4-binding molecules described in WO 2016/156570 may be attached or conjugated (directly or via a linker, as described herein) to the attachment point(s) or conjugation site(s) of the protein-based building block of the present technology.
CD-3-binding molecules, as described, e.g., in WO 2016/180982. Any of the CD-3-binding molecules described in WO 2016/180982 may be attached or conjugated (directly or via a linker, as described herein) to the attachment point(s) or conjugation site(s) of the protein-based building block of the present technology.
Glucocorticoid-induced Tumor Necrosis Factor (TNF) receptor-related protein (GITR)-binding molecules, as described, e.g., in WO 2017/068186. Any of the GITR-binding molecules described in WO 2017/068186 may be attached or conjugated (directly or via a linker, as described herein) to the attachment point(s) or conjugation site(s) of the protein-based building block of the present technology.
P2X purinoceptor 7 (P2X7)-binding molecules, as described, e.g., in WO 2017/081265. Any of the P2X7-binding molecules described in WO 2017/081265 may be attached or conjugated (directly or via a linker, as described herein) to the attachment point(s) or conjugation site(s) of the protein-based building block of the present technology.
CD38-binding molecules, as described, e.g., in WO 2017/081211. Any of the CD38-binding molecules described in WO 2017/081211 may be attached or conjugated (directly or via a linker, as described herein) to the attachment point(s) or conjugation site(s) of the protein-based building block of the present technology.
Macrophage migration inhibitory factor (MIF)-binding molecules, as described, e.g., in WO 2018/050833. Any of the MIF-binding molecules described in WO 2018/050833 may be attached or conjugated (directly or via a linker, as described herein) to the attachment point(s) or conjugation site(s) of the protein-based building block of the present technology.
Matrix Metallopeptidase 13 (MMP13)-binding molecules, as described, e.g., in WO 2018/220235 or WO 2018/220236. Any of the MMP13-binding molecules described in WO 2018/220235 or WO 2018/220236 may be attached or conjugated (directly or via a linker, as described herein) to the attachment point(s) or conjugation site(s) of the protein-based building block of the present technology.
A disintegrin and metalloproteinase with thrombospondin motifs (ADAMTS)-binding molecules, as described, e.g., in WO 2018/220236. Any of the ADAMTS-binding molecules described in WO 2018/220236 may be attached or conjugated (directly or via a linker, as described herein) to the attachment point(s) or conjugation site(s) of the protein-based building block of the present technology.
Interleukin 13 (IL-13)-binding molecules, as described, e.g., in WO 2021/116182 or WO 2022/063984. Any of the IL-13-binding molecules described in WO 2021/116182 or WO 2022/063984 may be attached or conjugated (directly or via a linker, as described herein) to the attachment point(s) or conjugation site(s) of the protein-based building block of the present technology.
Thymic stromal lymphopoietin (TSLP)-binding molecules, as described, e.g., in WO 2021/116182. Any of the TSLP-binding molecules described in WO 2021/116182 may be attached or conjugated (directly or via a linker, as described herein) to the attachment point(s) or conjugation site(s) of the protein-based building block of the present technology.
Interleukin 6 (IL-6)-binding molecules, as described, e.g., in WO 2022/129572. Any of the IL-6-binding molecules described in WO 2022/129572 may be attached or conjugated (directly or via a linker, as described herein) to the attachment point(s) or conjugation site(s) of the protein-based building block of the present technology.
DR5-binding molecules, such as, e.g., DR5 ISVDs, as described, e.g., in WO 2011/098520, or as depicted, e.g., in SEQ ID NOs.: 183 or 184. Any of the DR5-binding molecules described in WO 2011/098520 or SEQ ID NOs.: 183 or 184 may be attached or conjugated (directly or via a linker, as described herein) to the attachment point(s) or conjugation site(s) of the protein-based building block of the present technology.
TLR7/8 agonist (resiquimod). Resiquimod or R848 (an imidazoquinoline compound), is a TLR-7 and TLR-8 agonist and a potent inducer of cytokines that activate immune cells. As such, Resiquimod possesses antiviral and antitumoral activity. The present inventors have demonstrated site-specific conjugation of resiquimod onto engineered cysteines to create a DAR of 3 on an ISVD-based building block (see example 5.3).

Hence, the present technology provides molecules as defined herein which comprises at least one protein-based building block with at least one resiquimod molecule attached to at least one attachment point or conjugation site.

For instance, the present technology provides one molecule as described herein which comprises at least one ISVD-based building block, as defined herein, with at least one resiquimod molecule attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one resiquimod molecule, through the at least one conjugation site or attachment point, comprised in the molecule of the present technology, does not specifically bind to any target, such as protein and/or non-protein molecules, including biomolecules, to which the ISVD precursor specifically binds.

For instance, the present technology provides one molecule as described herein which comprises at least one DARPin-based building block, as defined herein, with at least one resiquimod molecule attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one resiquimod molecule attached to at least one attachment point or conjugation site.

For instance, the present technology provides one molecule as described herein which comprises at least one building block based on a small globular protein, such as CKS1, as defined herein, with at least one resiquimod molecule attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one DARPin-based building block selected from SEQ ID NO.: 199, 97 and/or 98, preferably SEQ ID NO.: 97 or 98, and at least one, preferably more than one, such as two, or, preferably, three resiquimod molecules conjugated to the attachment points or conjugation sited of the DARPin-based building blocks. Preferably, the molecule further comprises a HLE moiety, such as an albumin-binding ISVD, e.g., SEQ ID NO.: 106. In one embodiment, the at least one resiquimod molecule is conjugated to the DARPin-based building block comprised in molecule ALB-1C_K27m (SEQ ID NO.: 200), ALB-3C_K27m_w1 (SEQ ID NO.: 173) and/or ALB-5C_K27m (SEQ ID NO.: 174), preferably ALB-3C_K27m_w1 and/or ALB-5C_K27m.

In one embodiment, the molecule of the present technology comprises at least one ISVD-based building block selected from SEQ ID NO.: 80, 81 or 175, preferably SEQ ID NO.: 80 or 81, and at least one, preferably more than one, such as two, or, preferably, three resiquimod molecules conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one resiquimod molecule is conjugated to the ISVD-based building block comprised in molecule T028100069 (SEQ ID NO.: 107), T028100070 (SEQ ID NO.: 108) and/or T028100075 (SEQ ID NO.: 176), preferably T028100069 and/or T028100070.

As described above, there are preferably more than one resiquimod molecules conjugated to the ISVD- and/or DARPin-derived building blocks, preferably 3 resiquimod molecules per ISVD-based building block.

### Imaging Moieties

As described above, the protein-based carrier building block may have attached or conjugated, via its one or more conjugation sites or attachment points one or more other groups, residues, moieties or binding units, optionally linked via one or more peptidic linkers, wherein said one or more other groups, residues, moieties or binding units are used for imaging purposes (“imaging moiety”). For instance, the molecule of the present technology may comprise one, two, three, four, five, six, seven, eight, nine, ten or more imaging moieties attached or conjugated to the at least one protein-based carrier building block.

Examples of imaging moieties are provided in Agdeppa E D, Spilker M E. A review of imaging agent development. AAPS J. 2009 June; 11 (2): 286-99. For instance, the imaging moiety present in the molecule of the present technology may be suitable for radiotherapy and for radio/fluorescence-guided cancer surgery. For instance, the imaging moiety may comprise radioactive isotopes that can be used for diagnostic and therapeutic proposes. For instance, the imaging moiety may be a contrast agent. For instance, the imaging moiety may be a non-radioactive medical isotope. For instance, the imaging moiety may include desferrioxamine (DFO), such as used for ⁸⁹Zirconium-DFO-labeling. For instance, the imaging moiety may be a fluorophore such as Alexa 647 or pHAb.

### Toxic Moieties or Drugs

As described above, the protein-based carrier building block may have attached or conjugated, via its one or more conjugation sites or attachment points one or more other groups, residues, moieties or binding units, optionally linked via one or more (e.g., cleavable or non-cleavable, peptidic or non-peptidic) linkers, wherein said one or more other groups, residues, moieties or binding units are able to impart certain toxicity to cells and/or tissues (“toxic moiety” or “drug”). For instance, the molecule of the present technology may comprise one, two, three, four, five, six, seven, eight, nine, ten or more toxic moieties attached or conjugated to the at least one protein-based carrier building block.

A toxic moiety which may be attached or conjugated to the protein-based carrier building block may belong to the “tubulin inhibitor” family (e.g., maytansinoids, auristatins, taxol derivates) or to the “DNA-modifying agents” family (e.g., calicheamicins, duocarymycins). They can also be antibiotics or enzymes. For a review, see Criscitiello C. et al., “Antibody-drug conjugates in solid tumors: a look into novel targets”, Hematol Oncol, 2021, 14:20.

A special class of cytotoxic compounds (or toxic moieties) are tubulin inhibitors such as the maytansinoid DM4 and the macrolide Cryptophycin, which stop cell division. These cytotoxic compounds may be conjugated to other molecules such as antibodies. Ideally, the payload should only be released in the tumor cell and have little bystander effects. The toxins are usually attached to the antibody of interest via stochastic conjugation via lysines (e.g., Trastuzumab-DM1, see, e.g., Sang H. et al., “Conjugation site analysis of lysine-conjugated ADCs”, Methods Mol Biol., 2020, 2078:235-250) or via site specific conjugation, the latter usually via the free thiol of endogenous cysteines, see, e.g., Nadkarni DV., “Conjugations to endogenous cysteine residues”, Methods Mol Biol., 2020, 2078:37-49.

The present technology provides non-targeting protein-based building blocks comprising attachment points or conjugation sites (e.g., with engineered surface-exposed cysteines) for conjugation of payloads. If needed, the cysteine conjugation can be combined with stochastic conjugation on solvent-accessible lysines for another type of cargo (e.g., one peptide on cysteines and pHAb and Alexa fluor on lysines, see Example 18, in particular molecule EGFR7D12-3C_hCKS1_c3-cMyc NLS, SEQ ID NO.: 215).

Hence, the present technology provides molecules as defined herein which comprises at least one protein-based building block with at least one cytotoxic compound or payload, such as DM4 and/or Cryptophycin molecule attached to at least one attachment point or conjugation site.

For instance, the present technology provides one molecule as described herein which comprises at least one ISVD-based building block, as defined herein, with at least one cytotoxic compound or payload, such as DM4 and/or Cryptophycin molecule attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one cytotoxic compound or payload, such as DM4 and/or Cryptophycin, through the at least one conjugation site or attachment point, comprised in the molecule of the present technology, does not specifically bind to any target, such as protein and/or non-protein molecules, including biomolecules, to which the ISVD precursor specifically binds.

For instance, the present technology provides one molecule as described herein which comprises at least one DARPin-based building block, as defined herein, with at least one cytotoxic compound or payload, such as DM4 and/or Cryptophycin molecule attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one cytotoxic compound or payload, such as DM4 and/or Cryptophycin molecule attached to at least one attachment point or conjugation site.

For instance, the present technology provides one molecule as described herein which comprises at least one building block based on a small globular protein, such as CKS1, as defined herein, with at least one cytotoxic compound or payload, such as DM4 and/or Cryptophycin molecule attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one DARPin-based building block selected from SEQ ID NO.: 199, 97 and/or 98, preferably SEQ ID NO.: 97 or 98, and at least one, preferably more than one, such as two, or, preferably, three DM4 and/or three Cryptophycin molecules conjugated to the attachment points or conjugation sited of the DARPin-based building blocks. Preferably, the molecule further comprises a HLE moiety, such as an albumin-binding ISVD, e.g., SEQ ID NO.: 106. In one embodiment, the at least one DM4 and/or at least one Cryptophycin molecule is conjugated to the DARPin-based building block comprised in molecule ALB-1C_K27m (SEQ ID NO.: 200), ALB-3C_K27m_w1 (SEQ ID NO.: 173) and/or ALB-5C_K27m (SEQ ID NO.: 174), preferably ALB-3C_K27m_w1 and/or ALB-5C_K27m.

In one embodiment, the molecule of the present technology comprises at least one ISVD-based building block selected from SEQ ID NO.: 80, 81 or 175, preferably SEQ ID NO.: 80 or 81, and at least one, preferably more than one, such as two, or, preferably, three DM4 and/or three Cryptophycin molecules conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one DM4 and/or at least one Cryptophycin molecule is conjugated to the ISVD-based building block comprised in molecule T028100069 (SEQ ID NO.: 107), T028100070 (SEQ ID NO.: 108) and/or T028100075 (SEQ ID NO.: 176), preferably T028100069 and/or T028100070.

As described above, there are preferably more than one DM4 and/or at least one Cryptophycin molecule conjugated to the ISVD- and/or DARPin-derived building blocks, preferably 3 DM4 and/or 3 Cryptophycin molecules per ISVD-based building block.

Nucleic Acids Such as siRNA

The protein-based carrier building block may also have attached or conjugated, via its one or more conjugation sites or attachment points one or more siRNA molecules. For instance, the molecule of the present technology may comprise one, two, three, four, five, six, seven, eight, nine, ten or more siRNA molecules attached or conjugated to the at least one protein-based carrier building block.
siRNAs are a kind of macromolecular drugs that belong to the small non-encoding ribonucleic acid (sncRNA) family which can target and silence aimed gene expression such as oncogenes, tumor suppressor genes and other regulatory genes by specific sequence recognition with mRNA. For instance, FDA-approved siRNA include patisiran, givosiran, lumasiran and inclisiran (Cao W. et al., “Antibody-siRNA conjugates (ARC): Emerging siRNA drug formulation”, Medicine in drug discovery, 2022, 15:100128).

### Vitamins

Vitamins are also suitable cargos to be attached to the conjugation sites or attachment points present in the at least one protein-based building block comprised in the molecule of the present technology. Non-limiting examples of vitamins are folate (folic acid), biotin, vitamin C, etc.

### Folate

The folate receptor (FOLR) constitutes a useful target for tumor specific drug delivery, primarily because it is upregulated in many different types of cancers including those of ovary, endometrium, lung, kidney, mesothelium, head and neck. In normal human tissues, FOLR has very limited distribution mainly restricted to the kidneys, lungs, choroid plexus, and placenta. The receptors in these tissues except the placenta are localized on surface facing away from blood. These attributes make folate receptors an attractive target for efficient and selective binding. See Parashar S. et al., “A clickable folic acid-rhamnose conjugate for selective binding to cancer cells”, Results in Chemistry, 2022, 4:100409. Hence, the protein-based carrier building block may also have attached or conjugated, via its one or more conjugation sites or attachment points one or more folic acid (folate) molecules. For instance, the molecule of the present technology may comprise one, two, three, four, five, six, seven, eight, nine, ten or more folate molecules attached or conjugated to the at least one protein-based carrier building block.

Hence, folic acid (folate) may be attached or conjugated (directly or via a linker, as described herein) to the attachment point(s) or conjugation site(s) of the protein-based building block of the present technology.

The present technology therefore provides molecules as defined herein which comprises at least one protein-based building block with at least one folate molecule attached to at least one attachment point or conjugation site.

For instance, the present technology provides one molecule as described herein which comprises at least one ISVD-based building block, as defined herein, with at least one folate attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one folate, through the at least one conjugation site or attachment point, comprised in the molecule of the present technology, does not specifically bind to any target, such as protein and/or non-protein molecules, including biomolecules, to which the ISVD precursor specifically binds.

For instance, the present technology provides one molecule as described herein which comprises at least one DARPin-based building block, as defined herein, with at least folate attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one folate attached to at least one attachment point or conjugation site.

For instance, the present technology provides one molecule as described herein which comprises at least one building block based on a small globular protein, such as CKS1, as defined herein, with at least one folate attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one ISVD-based building block selected from SEQ ID NO.: 81 or 175, preferably SEQ ID NO.: 81, and at least one, preferably more than one, such as two, or, preferably, three folate molecules conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one folate is conjugated to the ISVD-based building block comprised in molecule T028100070 (SEQ ID NO.: 108) and/or T028100075 (SEQ ID NO.: 176), preferably T028100070. As described above, there are preferably more than one folate conjugated to the ISVD-derived building blocks, preferably 3 folate or more molecules per ISVD-based building block.

### Toll-Like Receptor (TLR) Agonists

Toll-like receptor (TLR) agonists may be a promising approach to the treatment of autoimmune diseases, some cancers, bacterial, and viral infections (Farooq M. et al., “Toll-like receptors as a therapeutic target in the era of immunotherapies”, Front. Cell Dev. Biol., 2021). Table 1 on Farooq M. et al. provides a list of TLR-based ligands in clinical trials. The protein-based carrier building block may also have attached or conjugated, via its one or more conjugation sites or attachment points one or more TLR agonists. For instance, the molecule of the present technology may comprise one, two, three, four, five, six, seven, eight, nine, ten or more TLR agonists attached or conjugated to the at least one protein-based carrier building block.

### Glycans

Targeted protein degradation strategies are gaining importance for new therapeutic strategies. Lysosome-targeting chimeras (LYTACs) make use of receptors, such as the cation-independent mannose 6-phosphate receptor (CI-M6PR) to direct extracellular proteins to lysosomes. See, e.g., Ahn G. et al., “Elucidating the cellular determinants of targeted membrane protein degradation by lysosome-targeting chimeras”, Science, 2023, 382 (6668): eadf6249 or Stevens CM. et al., “Development of oligomeric mannose-6-phosphonate conjugates for targeted protein degradation”, ACS Med Chem Lett., 2023, 14 (6): 719-726.

Hence, the present technology provides molecules as defined herein which comprises at least one protein-based building block with at least one glycan attached to at least one attachment point or conjugation site. Preferably, the glycan is mannose 6 phosphate (M6P or bisM6P). In this case, the molecule of the present technology, comprising one or more M6P (or bisM6P) can be targeted to the lysosome.

For instance, the present technology provides one molecule as described herein which comprises at least one ISVD-based building block, as defined herein, with at least one glycan, preferably a M6P or bisM6P molecule attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least one M6P or bisM6P molecule, through the at least one conjugation site or attachment point, comprised in the molecule of the present technology, does not specifically bind to any target, such as protein and/or non-protein molecules, including biomolecules, to which the ISVD precursor specifically binds.

For instance, the present technology provides one molecule as described herein which comprises at least one DARPin-based building block, as defined herein, with at least one glycan, preferably a M6P or bisM6P molecule attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one glycan, preferably a M6P or bisM6P molecule attached to at least one attachment point or conjugation site.

For instance, the present technology provides one molecule as described herein which comprises at least one building block based on a small globular protein, such as CKS1, as defined herein, with at least one glycan, preferably a M6P or bisM6P molecule attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one ISVD-based building block selected from SEQ ID NO.: 80, 81 or 175, preferably SEQ ID NO.: 80 or 81, and at least one, preferably more than one, such as two, or, preferably, three glycans or more, preferably M6P or bisM6P, conjugated to the attachment points or conjugation sited of the ISVD-based building blocks. Preferably, the molecule further comprises a HLE moiety, such as an albumin-binding ISVD, e.g., SEQ ID NO.: 106. In one embodiment, the at least one glycan, preferably M6P or bisM6P molecule is conjugated to the ISVD-based building block comprised in molecule T028100069 (SEQ ID NO.: 107), T028100070 (SEQ ID NO.: 108) and/or T028100075 (SEQ ID NO.: 176), preferably T028100069 and/or T028100070. As described above, there are preferably more than one glycans, preferably M6P or bisM6P, conjugated to the ISVD-derived building blocks, preferably 3 or more glycans, preferably M6P or bisM6P, per ISVD-based building block.

### Lipids

The conjugation of lipids to drug-comprising molecules may have several advantages, such as increase in lipophilicity, change of other properties of drugs, improved targeting to the lymphatic system, enhanced tumour targeting, enhanced cell internalization and reduced toxicity, see, e.g., Irby, D. et al., “Lipid-drug conjugate for enhancing drug delivery”, Mol. Pharmaceutics, 2017, (14) 5:1325-1338.

The present technology further provides molecules as defined herein which comprises at least one protein-based building block with at least one lipid attached to at least one attachment point or conjugation site. The lipid is preferably a fatty acid. The at least one lipid may be a short-chain fatty acid, a medium-chain lipid or a lipid with larger chain. The lipid may be saturated, unsaturated or partially saturated. The lipid may be branched or unbranched. For instance, the lipid may be selected from:
Hexanoic acid, which is the carboxylic acid derived from hexane with the chemical formula CH₃(CH₂)₄COOH, comprising 6 C atoms and a carboxylic head group;
Undecanoic acid, which is a carboxylic acid with chemical formula CH₃(CH₂)₉COOH.

For instance, the present technology provides one molecule as described herein which comprises at least one ISVD-based building block, as defined herein, with at least one lipid (preferably fatty acid) attached to at least one attachment point or conjugation site. For instance, the ISVD-based building block comprises or, alternatively, consists of a building block selected from SEQ ID NO.: 80-95, 175, 185, 186, 206, 222-224, or a sequence which has 80% or more identity with SEQ ID NO.: 80-95, 175, 185, 186, 206, 222-224, preferably a sequence which has 85% or more, 90% or more, 95% or more, 97% or more or 99% or more sequence identity with SEQ ID NO.: 80-95, 175, 185, 186, 206, 222-224, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, preferably does not specifically binds to any non-human protein to which it originally bound, such as bacterial and/or viral proteins, as described in detail above and/or preferably does not specifically binds to any non-protein molecule to which it originally bound, if any, all as described in detail above. Preferably, as described above, at least one ISVD-derived protein-based building block, preferably when conjugated to at least lipid, through the at least one conjugation site or attachment point, comprised in the molecule of the present technology, does not specifically bind to any target, such as protein and/or non-protein molecules, including biomolecules, to which the ISVD precursor specifically binds

For instance, the present technology provides one molecule as described herein which comprises at least one DARPin-based building block, as defined herein, with at least one lipid (preferably fatty acid) attached to at least one attachment point or conjugation site. For instance, the DARPin-based building block comprises or, alternatively, consists of a building block selected from SEQ ID NO.: 96-98, 181, 182, 188, 189, 199 or 208, or a sequence which has 80% or more identity with SEQ ID NO.: 96-98, 181, 182, 188, 189, 199 or 208, preferably a sequence which has 85% or more, 90% or more, 95% or more, 97% or more or 99% or more sequence identity with SEQ ID NO.: 96-98, 181, 182, 188, 189, 199 or 208, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein, in particular it does not specifically bind human KRAS protein, as described in detail above.

For instance, the present technology provides one molecule as described herein which comprises at least one affitin-based building block and/or at least one affibody-based building block, as defined herein, with at least one lipid (preferably fatty acid) attached to at least one attachment point or conjugation site.

For instance, the present technology provides one molecule as described herein which comprises at least one building block based on a small globular protein, such as CKS1, as defined herein, with at least one lipid (preferably fatty acid) attached to at least one attachment point or conjugation site. For instance, the building block may be a CSK-derived building block selected from SEQ ID NO.: 99-105, 191, 192 and 205, or a sequence which has 80% or more identity with SEQ ID NO.: 99-105, 191, 192 and 205, preferably a sequence which has 85% or more, 90% or more, 95% or more, 97% or more or 99% or more sequence identity with SEQ ID NO.: 99-105, 191, 192 and 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

In one embodiment, the molecule of the present technology comprises at least one building block selected from SEQ ID NO.: 86 (ISVD-derived, with 6 Cys) and 101 (CKS-derived, comprising 3 Cys), and at least one, preferably more than one, such as two, or, preferably, three lipids (preferably fatty acid) conjugated to the attachment points or conjugation sites of the building blocks. For example, the molecule may further comprise a HLE moiety, such as an albumin-binding ISVD, e.g., SEQ ID NO.: 106. In one embodiment, the at least one lipid (preferably fatty acid, even more preferably short-chain fatty acid) is conjugated to the ISVD-based building block comprised in molecule T028100078 (SEQ ID NO.: 113). As described above, there are preferably more than one lipid (preferably fatty acid, even more preferably short-chain fatty acid) conjugated to the building blocks, preferably 3 lipids (preferably fatty acid) molecules per building block.

### Nucleic Acid Molecules

The present technology also provides a nucleic acid molecule encoding the protein-based carrier building block and/or the molecule (or part of the molecule) of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable (non-human) hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the protein-based carrier building block and/or the molecule (or part of the molecule) of the present technology is also encompassed by the present technology.

A nucleic acid may be for example DNA, RNA, or a hybrid thereof, and may also comprise (e.g., chemically) modified nucleotides, like PNA. It can be single- or double-stranded. In one embodiment, it is in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly) peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### Vectors

Also provided is a vector comprising the nucleic acid molecule encoding the protein-based carrier building block and/or the molecule (or part of the molecule) of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors comprise at least one nucleic acid that is optionally linked to one or more regulatory elements, such as for example one or more suitable promoter(s), enhancer(s), terminator(s), etc.). In one embodiment, the vector is an expression vector, i.e. a vector suitable for expressing an encoded polypeptide or construct under suitable conditions, e.g. when the vector is introduced into a (e.g. human) cell. DNA-based vectors include the presence of elements for transcription (e.g., a promoter and a polyA signal) and translation (e.g. Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### Compositions

The present technology also provides a composition comprising the protein-based carrier building block and/or the molecule of the present technology. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### Host Organisms

The present technology also pertains to host cells or host organisms expressing the protein-based carrier building block and/or the molecule (or part of the molecule) of the present technology, comprising the nucleic acid encoding the protein-based carrier building block and/or the molecule (or part of the molecule) of the present technology, and/or the vector comprising the nucleic acid molecule encoding the protein-based carrier building block and/or the molecule (or part of the molecule) of the present technology.

In one embodiment the host is a non-human host. Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Komagataella phaffii (Pichia pastoris, see Bernauer L., et al. (“Komagataella phaffii as emerging model organism in fundamental research”, Front. Microbiol., 2021, 11:1-16)). In one embodiment, the host is Komagataella phaffii (Pichia pastoris). In another embodiment, the host is Escherichia coli. Of course, cell free systems may also be employed to produce the protein-based carrier building block and/or the molecule of the present technology, as reviewed, for instance, in Gregorio N E, Levine M Z, Oza J P, “A user's guide to cell-free protein synthesis”, Methods Protoc. 2019, 2 (1): 24.

### Methods and Uses of the Molecule

The present technology also provides a method for producing the protein-based carrier building block and/or the molecule of the present technology. The method may comprise transforming/transfecting a host cell or host organism with a nucleic acid encoding the at least one protein-based carrier building block and/or the molecule (or part of the molecule), expressing the at least one protein-based carrier building block and/or the molecule (or part of the molecule) in the host, optionally followed by one or more isolation and/or purification steps. Specifically, the method may comprise:
a) expressing, in a suitable host cell or host organism or in another suitable expression system, a nucleic acid sequence encoding the at least one protein-based carrier building block and/or the molecule (or part of the molecule); optionally followed by:
b) isolating and/or purifying the at least one protein-based carrier building block and/or the molecule (or part of the molecule).

During expression in any suitable expression system, capping agents may be used in order to cap the at least one attachment point or conjugation site present in the protein-based carrier building block. For instance, if the at least one protein-based carrier building block comprises one or more —SH groups as attachment points or conjugation sites, cysteamine may be added during expression, in order to cap any free thiol group. Of course, the cap must be removed before attachment or conjugation of the cargo (e.g., with TCEP if the attachment point was capped with cysteamine).

For instance, the protein-based carrier building block and/or the molecule (or part of the molecule) of the present technology may be encoded in a nucleic acid together with a Protein A binding building block, so that the the protein-based carrier building block and/or the molecule (or part of the molecule) can be easily purified with Protein A chromatography after expression. Hence, Protein A chromatography can be employed to purify the protein-based carrier building block and/or the molecule (or part of the molecule). Further purification steps such as size exclusion chromatography (SEC) or ultrafiltration and/or ion-exchange chromatography may be applied in order to purify the protein-based carrier building block and/or the molecule (or part of the molecule).

To produce/obtain the at least one protein-based carrier building block and/or the molecule of the present technology, both in genetic fusion or as a single polypeptide, the host cell or host organism or cell free system may generally be kept, maintained and/or cultured under conditions such that the (desired) protein-based carrier building block and/or molecule of the technology is optimally expressed/produced. Suitable conditions will be clear to the skilled person and will usually depend upon the host cell/host organism or cell free system used, as well as on the regulatory elements that control the expression of the protein-based carrier building block or molecule of the present technology.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Komagataella phaffii (Pichia pastoris). In one embodiment, the host is Komagataella phaffii (Pichia pastoris). In another embodiment, the host is Escherichia coli.

Hence, the at least one protein-based building block and/or the molecule (or part of the molecule) of the present technology can be encoded in a nucleic acid molecule, optionally as part of an expression vector, and expressed and produced recombinantly, as described above.

In one embodiment, the molecule of the present technology comprises more than one protein-based carrier building block as defined above. For instance, the molecule of the present technology may comprise 2, 3 or more carrier building blocks as defined above. The more than one protein-based carrier building block of the present technology can be encoded in a single nucleic acid molecule, optionally as part of an expression vector, and expressed and produced recombinantly, as described above.

In addition to the at least one protein-based carrier building block, the molecule of the present technology may further comprise one or more other groups, residues, moieties or binding units in which said one or more other groups, residues, moieties or binding units provide the molecule with several functionalities, such as binding specificity (e.g., by the presence of a targeting moiety in the molecule of the present technology), increased (in vivo) half-life extension (e.g., by the presence of half-life extending moiety in the molecule of the present technology), therapeutic properties (e.g., by the presence of a pharmaceutically active moiety in the molecule of the present technology), etc.

The one or more protein-based building blocks and/or the at least one cargo comprised in the molecule of the present technology may be recombinantly expressed as part of one or more genetic construct(s) and/or may be independently chemically synthesized (e.g., by SPPS). For instance, one or more protein-based carrier building block(s) may be expressed recombinantly, as part of a single genetic construct, and the one or more cargo(s) may also be expressed as part of another genetic construct. In a further step, the one or more cargo(s) may be attached or conjugated to the at least one protein-based carrier building block(s) through the conjugation site(s) or attachment point(s).

The molecule of the present technology may comprise at least one protein-based carrier building block and one or more other groups, residues, moieties or binding units, wherein the at least one protein-based carrier building block and the one or more other groups, residues, moieties or binding units are part of a single genetic construct and expressed recombinantly, i.e., they are expressed recombinantly as a single polypeptide. Further groups, residues, moieties or binding units may then be attached or conjugated to one or more conjugation site(s) or attachment point(s) present in the protein-based carrier building block(s).

For instance, the molecule of the present technology may comprise two or more protein-based carrier building blocks which may be part of a genetic construct and recombinantly expressed.

For instance, the molecule of the present technology may comprise one or more protein-based carrier building block and one or more other groups, residues, moieties or binding units in which said one or more other groups, residues, moieties or binding units provide the molecule with several functionalities, such as binding specificity, increased (in vivo) half-life extension, therapeutic properties etc. In this case, the one or more protein-based carrier building block and the one or more other groups, residues, moieties or binding units may be part of a single genetic construct and recombinantly expressed as a single polypeptide.

For instance, the molecule of the present technology may comprise one protein-based carrier building block and one half-life extension moiety, wherein the half-life extension moiety and protein-based carrier building block may be part of a genetic construct and expressed recombinantly as a single polypeptide.

Hence, the molecule of the present technology may comprise or consist of more than one protein-based building block(s), which may be part of a genetic construct and expressed recombinantly as a single polypeptide. One or more cargos, as defined above, may then be attached or conjugated to one or more attachment points or conjugation sites present in the protein-based building blocks.

In another embodiment, the molecule of the present technology may comprise one or more protein-based building block(s) and one or more half-life extending moieties, as described above, they may be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s) and one or more targeting moieties, as described above, and they may all be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s) and one or more therapeutic moieties, as described above, and they may all be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s) and one or more targeting and/or therapeutic moiety, as described above, and they may all be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s), one or more half-life extending moiety and one or more targeting moiety, as described above, and they may all be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s), one or more half-life extending moiety and one or more therapeutic moiety, as described above, and they may all be part of a genetic construct and expressed recombinantly as a single polypeptide. One or more further cargos, as defined above, may then be attached or conjugated to one or more attachment points or conjugation sites present in the protein-based building block(s).

In another embodiment, the molecule of the present technology may comprise one or more protein-based building block(s), one or more half-life extending moiety and/or one or more targeting and/or therapeutic moiety, as described above, and they may all be part of a genetic construct and expressed recombinantly as a single polypeptide. One or more further cargos, as defined above, may then be attached or conjugated to one or more attachment points or conjugation sites present in the protein-based building block(s).

Alternatively, the at least one protein-based carrier building block and/or molecule (or part of the molecule) of the present technology can be produced synthetically, e.g., using solid-phase peptide synthesis (SPPS), see, e.g., Jaradat, D. M. M., Thirteen decades of peptide synthesis: key developments in solid phase peptide synthesis and amide bond formation utilized in peptide ligation, Amino Acids 50, 39-68 (2018).

As it will be evident to the skilled reader, if the molecule of the present technology comprises one or more protein-based building blocks and, optionally, one or more other groups, residues, moieties or binding units, as defined above, part or the whole molecule may be encoded in a nucleic acid molecule, optionally as part of an expression vector, as defined above, and part or the whole molecule may be produced synthetically.

Once the one or more protein-based building blocks and, optionally, the one or more other groups, residues, moieties or binding units, as defined above, are produced, the cargos may be attached to the at least one protein-based building block via the attachment points or conjugation sites (preferably engineered conjugation sites or attachment points), as described above. For instance, the at least one protein-based carrier building block may be expressed recombinantly, as described above, and the cargo(s) conjugated to it via the at least one attachment point or conjugation site, thus rendering the molecule of the present technology. For instance, the at least one protein-based carrier building block may be produced synthetically, e.g., using SPPS, as described above, and the cargo(s) conjugated to it via the at least one attachment point or conjugation site, thus rendering the molecule of the present technology. For instance, the at least one protein-based carrier building block and one or more further moieties, such as HLE moieties, may be encoded in an expression vector and be expressed recombinantly, as described above, and the cargo(s) conjugated to the protein-based carrier building block via the at least one attachment point or conjugation site, thus rendering the molecule of the present technology. The at least one protein-based carrier building block and one or more further moieties, such as HLE moieties may be linked through a linker, as described in detail above.

For instance, the at least one protein-based carrier building block may be expressed recombinantly (alone or together with, e.g., at least some of the half-life extension moieties), as described above, and the cargo(s) conjugated to it via the at least one attachment point or conjugation site. If a conjugation site is a —SH group (free or capped) present in the side chain of a cysteine present in the recombinantly-expressed protein-based carrier building block (which may be expressed alone or together with, e.g., at least some of the half-life extension moieties, as explained herein), the cargo can be attached or conjugated to the building block (directly or by means of a linker) by alkylation, metal-assisted arylation, disulphide exchange or addition to a maleimide Michael acceptor, see above in this description for further details. If a conjugation site is a-OH group of a tyrosine present in the recombinantly-expressed protein-based carrier building block (which may be expressed alone or together with, e.g., at least some of the half-life extension moieties, as explained herein), the cargo can be attached or conjugated to the building block (directly or by means of a linker) by several chemical methods such as cross-linking via catalytic tyrosine mono electronic oxidation, three-component Mannich-type tyrosine conjugation, conjugation via sulphur fluoride exchange chemistry (SuFEx), transition-metal complexes for tyrosine conjugation, diazonium coupling reaction, reactions with triazolinediones, etc. (for a review, see, e.g., D. Alvarez Dorta et al., Chem. Eur. J., 2020, 26, 14257). If a conjugation site is the —OH group of an N- and/or C-terminal tyrosine present in the recombinantly-expressed protein-based carrier building block (which may be expressed alone or together with, e.g., at least some of the half-life extension moieties, as explained herein), the cargo can be attached or conjugated to the building block (directly or by means of a linker) enzymatically as described, e.g., in Alan M. Marmelstein et al., Journal of the American Chemical Society, 2020, 142 (11), 5078-5086. If a conjugation site is the N-terminal primary amine of the recombinantly-expressed protein-based carrier building block and/or the primary amine present in the side chain of an amino acid present in the recombinantly-expressed protein-based carrier building block (which may be expressed alone or together with, e.g., at least some of the half-life extension moieties, as explained herein) (e.g., Lys, Orn, or any non-natural amino acid with a primary amine on its side chain), the cargo may be attached or conjugated to the carrier building block (directly or by means of a linker) by reaction of a group present in the cargo/linker (e.g., isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, or fluorophenyl esters) and the primary amine.

The skilled person is familiar with groups, residues, or moieties able to provide therapeutic properties to the molecule of the present technology, such as pharmaceutically active moieties. The skilled person is also familiar with groups, residues or moieties able to provide specific targeting of the molecule of the technology to desired organs/tissues/cells in the human or animal body, such as targeting moieties.

For instance, at least some of the half-life extension moieties, targeting moieties, therapeutic moieties or precursors therefrom described above in the “Cargos” section may be incorporated in the molecule of the present technology as part of a genetic construct, expressed recombinantly, possibly together with the at least one protein-based carrier building block, as described in detail above. Hence, at least some of the half-life extension moieties, targeting moieties, therapeutic moieties or precursors therefrom or imaging molecules described above in the “Cargos” section may be incorporated in the molecule of the present technology (i) by attaching or conjugating them to the at least one attachment point or conjugation site present in the protein-based carrier building block, or (ii) by expressing them recombinantly together with the protein-based carrier building block. Of course, combinations of the above mechanisms are possible; for instance, one or more of the half-life extension moieties, targeting moieties, therapeutic moieties or precursors therefrom described above in the “Cargos” section may be incorporated in the molecule of the present technology as part of a genetic construct, expressed recombinantly possibly together with the at least one protein-based carrier building block, and/or one or more of the half-life extension moieties, targeting moieties, therapeutic moieties or precursors therefrom described above in the “Cargos” section may be incorporated in the molecule of the present technology by attaching or conjugating them to the at least one attachment point or conjugation site present in the protein-based carrier building block. The skilled person will understand and decide how to generate the molecule of the present technology in light of the number of protein-based carrier building blocks and specific moieties and/or cargos that the molecule will incorporate.

If two or more proteins (e.g., one ISVD-derived protein-based carrier building block, and one further protein, such as one ISVD, which may be a targeting moiety, which may increase the in vivo half-life of the protein-based building block and/or molecule of the present technology and/or which may have therapeutic properties; or one CSK-based carrier building block and one further protein which may be a targeting moiety, which may increase the in vivo half-life of the protein-based building block and/or molecule of the present technology and/or which may have therapeutic properties; or one DARPin-based carrier building block and one further protein, which may be a targeting moiety, which may increase the in vivo half-life of the protein-based building block and/or molecule of the present technology, and/or which may have therapeutic properties) are comprised in the molecule of the present technology, they may be directly linked to each other, and/or may be linked to each other via one or more suitable linkers, or any combination thereof. Suitable linkers have been described above in this description.

As already described, the conjugation of cargos to the attachment points or conjugation sites may be performed directly or via a linker. Suitable linkers are, for instance, APN-Maleimide linker (806536, Sigma-Aldrich), which is a bifunctional linker (see also Formula I below). This linker allows for conjugation twice, via cysteine-based chemistry. Both APN and maleimide couple to free thiols, albeit at different speed. An example is shown in FIG. 4. Hence, the APN-Maleimide linker may be used to attach cargos to attachment points or conjugation sites which are —SH groups present, e.g., in the side chain of a cysteine. Another linker is, for instance, N-ethylmaleimide (see, e.g., Formula II) or Maleimido-PEG12-acid (PubChem CID 68757103, UPAC name 3-[2-[2-[2-[2-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]ethoxy]ethoxy]ethoxy]ethoxy]propanoic acid, also known as “Mal-amido-PEG12-acid”), see Example 5.3.

The present technology further provides a method for producing the protein-based building block of the present technology, wherein the method comprises:
a. Providing a protein-based building block precursor, a defined herein;
b. Generating at least one, preferably more, as described herein, attachment points or conjugation sites in the protein-based building block precursor provided in a., as described in detail herein, and/or eliminating the specific binding properties of the protein-based building block precursor provided in a. to produce the protein-based building block of the present technology, wherein the protein-based building block:
a) comprises at least one conjugation site or attachment point, preferably at least two attachment points or conjugation sites;
b) has a molecular mass of about 2.5 to about 70 kDa, preferably of about 2.5 to about 50 kDa, such as from about 2.5 kDa to less than 50 kDa, more preferably of about 2.5 to about 30 kDa, even more preferably of about 2.5 to about 16 kDa;
c) has a globular 3D structure;
d) has a solubility of 10 mg/ml or more, measured in an aqueous solution at RT, preferably measured in a buffer or water at RT, more preferably in a buffer such as citrate buffer or phosphate-buffered saline (PBS) at pH 7.0 or 7.4, at RT, or histidine buffer at pH 6.5, at RT (comprising histidine (10 mM to 100 mM, such as 10 mM), sucrose (1% to 10%, such as 10%) and, optionally, Tween 80 (0.001% to 1%, such as 0.01%)), or phosphate buffer pH 7.0, at RT (comprising NaH₂PO₄/Na₂HPO₄ (10 and 50 mM, such as 10 mM), sodium chloride (NaCl) (100-150 mM, such as 130 mM NaCl) and, optionally, Tween 80 (0.001% to 1%, such as 0.01%));
e) does not specifically bind to any human protein or binds one or more human proteins with a K_{D} (K_{D} value) greater than 5×10⁻⁴ mol/litre, preferably as determined by cell-binding assay or by surface plasmon resonance (SPR), for instance as described herein and/or in Ober et al. 2001, Intern. Immunology 13:1551-1559, or does not specifically bind to any human cell or binds one or more human cells with a K_{D} (K_{D} value) greater than 5×10⁻⁴ mol/litre, preferably as determined by cell-binding assay or by SPR;
f) optionally, does not specifically bind to any (non-human) molecule which the protein-based carrier building block precursor specifically binds to, such as protein F of RSV, or binds to any (non-human) molecule which the protein-based carrier building block precursor specifically binds to, such as protein F of RSV, with a K_{D} (K_{D} value) greater than 5×10⁻⁴ mol/litre, preferably as determined by cell-binding assay or by SPR;
g) optionally, does not specifically bind to any human cell and/or cell type, or binds to a human cell and/or cell type with a K_{D} (K_{D} value) greater than 5×10⁻⁴ mol/litre, preferably as determined by cell-binding assay;
h) optionally, does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein;
i) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein;
j) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein when it has at least one cargo attached to it (via the at least one conjugation sites or attachment points comprised therein);
k) optionally, does not comprise or consists of an amino acid sequence selected from SEQ ID NO.: 1-34 as depicted on Tables A-1 and A-2 of WO 2016/055656 and/or SEQ ID NO.: 1-12 as depicted on Table A-1 of WO 2010/139808; and
l) optionally, does not comprise or consists of the amino acid sequence as defined in SEQ ID NO.: 214.

The molecule of the present technology or the composition comprising the molecule of the present technology are useful as a medicament.

Accordingly, the present technology provides the molecule of the present technology or a composition comprising the molecule of the present technology for use as a medicament.

Also provided is the molecule of the present technology or a composition comprising the molecule of the present technology for use in the (prophylactic and/or therapeutic) treatment.

Also provided is the molecule of the present technology or a composition comprising the molecule of the present technology for use in the (prophylactic and/or therapeutic) treatment of an autoimmune/inflammatory disease and/or cancer, such as hematological (blood) and solid tumor cancer disease.

Also provided is the molecule of the present technology or a composition comprising the molecule of the present technology for use in the (prophylactic and/or therapeutic) treatment of an infectious disease.

Also provided is the molecule of the present technology or a composition comprising the molecule of the present technology for use as a vaccine. Hence, the present technology provides a vaccine comprising the molecule of the present technology or a composition comprising the molecule of the present technology, optionally further comprising further components such as pharmaceutically acceptable carriers and/or adjuvants.

A “subject” as referred to in the context of the present technology can be any animal. In one embodiment, the subject is a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal. In one embodiment, the subject is a human subject.

Substances, including molecules or compositions may be administered to a subject by any suitable route of administration, for example by enteral (such as oral or rectal) or parenteral (such as epicutaneous, sublingual, buccal, nasal, intratracheal, intra-articular, intradermal, intramuscular, intraperitoneal, intravenous, subcutaneous, transdermal, or transmucosal) administration. In one embodiment, substances are administered by parenteral administration, such as intramuscular, subcutaneous or intradermal, administration.

An effective amount of a molecule as described, or a composition comprising the molecule of the present technology can be administered to a subject in order to provide the intended treatment results.

One or more doses can be administered. If more than one dose is administered, the doses can be administered in suitable intervals in order to maximize the effect of the molecule or composition comprising the same.

### EXAMPLES

### Example 1. In Silico Design of ISVD-Based Carrier Building Blocks

ISVD RSV001A04 (SEQ ID NO.: 179, also referred to as RSV 001A04) was selected as starting point for developing the ISVD-based carrier building block. FIG. 1 shows the amino acid sequence of ISVD RSV001A04 (SEQ ID NO.: 179). Using MAESTRO, residues in the building block precursor with a Solvent-Accessible Surface Area (SASA) greater than or equal to 27 Å² (square angstrom) were considered to be solvent-accessible and further considered to calculate stability of a mutation to a cysteine. As a result, 78 potential solvent-accessible residues were selected in SEQ ID NO.: 179 as potential positions for point mutations with cysteines, to generate conjugation sites or attachment points in the protein-based carrier building block. These positions are (in SEQ ID NO.: 179, Kabat numbering):
1, 3, 5, 7-8, 10-15, 17-19, 21, 23, 25-28, 30-32, 39, 41-46, 52a-59, 61-62, 64-66, 68, 70-76, 79, 81, 82a-82b, 83-85, 87, 89, 91, 96, 98-100a, 100d-100g, 101-103, 105-106, 108, 110 and 112-113.

Stability (ΔG in solvent) of each mutant was calculated using MAESTRO, see, e.g., Laimer J. et al, “MAESTRO—multi agent stability prediction upon point mutations”, BMC Bioinformatics, 2015, 16:116, for further details. Destabilizing cysteine mutations (i.e., those with higher calculated ΔG in solvent) were not further considered as potential positions for conjugation sites or attachment points. Based on the stability data, 27 potential positions were further selected; see the following amino acid positions in SEQ ID NO.: 179 according to Kabat numbering:
E1, S7, Q13, S17, S19, S21, A23, S25, G26, S28, N31, K43, E44, D55, N62, G65, T68, S70, D72, A74, K75, S82b, D85, G100a, D100f, R105, S112.

In addition, the —SH group of an engineered C-terminal Cys (i.e., not present in the building block precursor) preceded by a GG tag was also selected as potential attachment point (-GGC).

The following potential solvent-accessible positions were finally selected (in SEQ ID NO.: 179, according to Kabat numbering) as preferred combinations of potential solvent-accessible positions based on the in-silico predictions:

### 9×CYS

*S21, N31, K43, T68, D72, S82b, G100a, D100f, R105
*S7, Q13, S19, A23, E44, D55, N62, S70, A74
*S7, S17, N31, E44, D55, N62, T68, K75, S112.

### 6×CYS

*S19, E44, G65, S70, S82b, S112
*S21, K43, D55, T68, A74, S112
*S19, A23, N31, S70, S82b, D100f
*Q13, S25, K43, G65, D72, G100a
*S25, K43, K75, S82, G100a, S112C
*S25, K43, K75, G100a, R105, S112
*S25, K43, K75, G100a, R105 and C-terminal C (-GGC)
*K43, T68, K75, G100a, R105 and C-terminal C (-GGC)
*S25, K43, K75, D100f, R105 and C-terminal C (-GGC)
*K43, T68, K75, D100f, R105 and C-terminal C (-GGC).

### 3×CYS

*K43, D100f, R105
*K43, K75, G100a
*S21, T68, D100f
*S7, E44, D55
*Q13, D72, G100a
*Q13, N31, D100f.

As a result, the following protein-based carrier building blocks comprising three attachment points or conjugation sites which are the —SH group in the side chain of three cysteines located at solvent-accessible positions were designed:
a) 13001, corresponding to SEQ ID NO.: 80, which comprises the following point mutations in SEQ ID NO.: 179, at the following positions (according to Kabat numbering): K43C, D100fC, R105C and Q108L;
b) 13002, corresponding to SEQ ID NO.: 81, which comprises the following point mutations in SEQ ID NO.: 179, at the following positions (according to Kabat numbering): K43C, K75C, G100aC and Q108L;
c) 13003, corresponding to SEQ ID NO.: 82, which comprises the following point mutations in SEQ ID NO.: 179, at the following positions (according to Kabat numbering): S21C, T68C, D100fC and Q108L;
d) 13004, corresponding to SEQ ID NO.: 83, which comprises the following point mutations in SEQ ID NO.: 179, at the following positions (according to Kabat numbering): Q13C, D72C, G100aC and Q108L;
e) 13005, corresponding to SEQ ID NO.: 84, which comprises the following point mutations in SEQ ID NO.: 179, at the following positions (according to Kabat numbering): Q13C, N31C, D100fC and Q108L; and
f) 13006, corresponding to SEQ ID NO.: 85, which comprises the following point mutations in SEQ ID NO.: 179, at the following positions (according to Kabat numbering): S7C, E44C, D55C and Q108L.

Mutation Q108L was performed in all cases as shown in Table 3 above. As control, the polypeptide of SEQ ID NO.: 175 was designed. In this polypeptide only mutation Q108L was introduced, together with a-GGC tag at the C-terminal.

In addition, the following protein-based carrier building blocks comprising six attachment points or conjugation sites which are the —SH group in the side chain of six cysteines located at solvent-accessible positions were designed:
a) 16001, corresponding to SEQ ID NO.: 86, which comprises the following point mutations in SEQ ID NO.: 179, at the following positions (according to Kabat numbering): S19C, E44C, G65C, S70C, S82bC, Q108L and S112C;
b) 16002, corresponding to SEQ ID NO.: 87, which comprises the following point mutations in SEQ ID NO.: 179, at the following positions (according to Kabat numbering): S21C, K43C, D55C, T68C, A74C, Q108L and S112C;
c) 16003, corresponding to SEQ ID NO.: 88, which comprises the following point mutations in SEQ ID NO.: 179, at the following positions (according to Kabat numbering): S19C, A23C, N31C, S70C, S82bC, D100fC and Q108L;
d) 16004, corresponding to SEQ ID NO.: 89, which comprises the following point mutations in SEQ ID NO.: 179, at the following positions (according to Kabat numbering): Q13C, S25C, K43C, G65C, D72C, G100aC and Q108L;
e) 16005, corresponding to SEQ ID NO.: 90, which comprises the following point mutations in SEQ ID NO.: 179, at the following positions (according to Kabat numbering): L11V, S25C, K43C, K75C, S82bC, V89L, G100aC, Q108L and S112C;
f) 16006, corresponding to SEQ ID NO.: 91, which comprises the following point mutations in SEQ ID NO.: 179, at the following positions (according to Kabat numbering): L11V, S25C, K43C, K75C, V89L, G100aC, R105C, Q108L and S112C;
g) 16007, corresponding to SEQ ID NO.: 92, which comprises the following point mutations in SEQ ID NO.: 179, at the following positions (according to Kabat numbering): L11V, S25C, K43C, K75C, V89L, G100aC, R105C, Q108L and a C-terminal-GGC tag;
h) 16008, corresponding to SEQ ID NO.: 93, which comprises the following point mutations in SEQ ID NO.: 179, at the following positions (according to Kabat numbering): L11V, K43C, T68C, K75C, V89L, G100aC, R105C, Q108L and a C-terminal-GGC tag;
i) 16009, corresponding to SEQ ID NO.: 94, which comprises the following point mutations in SEQ ID NO.: 179, at the following positions (according to Kabat numbering): L11V, S25C, K43C, K75C, V89L, D100fC, R105C, Q108L and a C-terminal -GGC tag; and
j) 16010, corresponding to SEQ ID NO.: 95, which comprises the following point mutations in SEQ ID NO.: 179, at the following positions (according to Kabat numbering): L11V, K43C, T68C, K75C, V89L, D100fC, R105C, Q108L and a C-terminal -GGC tag.

Mutation Q108L was performed in all cases as shown in Table 3 above. In some cases, mutations L11V and V89L were also performed to avoid binding by pre-existing antibodies.

### Example 2. In Silico Design of DARPin-Based Carrier Building Blocks

DARPin K27 was selected as starting point for developing the DARPin-based carrier building block. In particular, the polypeptide as defined in SEQ ID NO.: 187 was chosen as the building-block precursor in this case. Arginine residues at positions 69, 102 and 111 were mutated to alanine, so that the polypeptide no longer binds any human protein, in particular its original target protein, KRAS. In addition, the C-terminal leucine was removed. See FIG. 2 and SEQ ID NO.: 68. Using MAESTRO, residues in the building block precursor with a Solvent-Accessible Surface Area (SASA) greater than or equal to 27 Å² (square angstrom) were considered to be solvent-accessible and further considered to calculate stability of a mutation to a cysteine. As a result, the following potential well solvent-accessible residues were selected in SEQ ID NO.: 187 as potential positions for point mutations with cysteines, to generate conjugation sites or attachment points in the protein-based carrier building block:
1-2, 4-5, 8, 11-17, 19-20, 23-25, 27, 29, 31-34, 36, 44-49, 52, 56-58, 60, 62, 64, 66-67, 77-82, 85, 89-91, 93, 95, 97, 99-100, 107, 110-115, 118-119, 121-124, 126-128, 130, 132-135, 138-139, 142-148, 151-152 and 154-155.

In addition, the —SH group of an engineered C-terminal Cys (i.e., not present in the building block precursor) was also selected as potential attachment point or conjugation site.

Stability (ΔG in solvent) of each mutant was calculated using MAESTRO, see, e.g., Laimer J. et al, “MAESTRO—multi agent stability prediction upon point mutations”, BMC Bioinformatics, 2015, 16:116, for further details. Destabilizing cysteine mutations were not further considered as potential positions for conjugation sites or attachment points. Based on the stability data the following potential positions in SEQ ID NO.: 187 were further selected as potential solvent-accessible positions based on the in-silico predictions:
5, 49, 60, 64, 82, 85, 93, 95, 97, 100, 115, 126, 143, 148, 155 and C-terminal Cys.

The following potential solvent-accessible positions in SEQ ID NO.: 187 were finally selected as potential solvent-accessible positions based on the in-silico predictions:
85, 95, 143, 148 and C-terminal Cys.

As a result, the following protein-based carrier building blocks comprising one, three or five attachment points or conjugation sites which are the —SH group in the side chain of three or five cysteines located at solvent-accessible positions were designed:
a) 33001, corresponding to SEQ ID NO.: 96, which comprises the following point mutations in SEQ ID NO.: 68, at the following positions: D143C, D148C and a C-terminal Cys;
b) 33002, corresponding to SEQ ID NO.: 97, which comprises the following point mutations in SEQ ID NO.: 68, at the following positions: E85C, N95C and a C-terminal Cys; and
c) 35001, corresponding to SEQ ID NO.: 98, which comprises the following point mutations in SEQ ID NO.: 68, at the following positions: E85C, N95C, D143C, D148C and a C-terminal Cys.

As control, the polypeptides of SEQ ID NOs.: 197, 199 and 208 were designed. These polypeptides comprise a C-terminal Cys but do not comprise the Cys-point mutations in positions 85, 95, 143 and/or 148. In addition, the polypeptide of SEQ ID NOs.: 197 and 208 comprise a Leu before the C-terminal Cys. The polypeptide of SEQ ID NOs.: 197 comprises Arg at positions 69, 102 and 111, whereas the polypeptides of SEQ ID NOs.: 199 and 208 comprise Ala at positions 69, 102 and 111:

SEQ ID NO.: 199:
DLGKKLLEAARAGQDDEVRILMANGADVNAHDTFGFTPLHLAALYGHLE
IVEVLLKNGADVNADDSYGATPLHLAAMRGHLEIVEVLLKYGADVNAAD
EEGATPLHLAAKAGHLEIVEVLLKNGADVNAQDKFGKTAFDISIDNGNE
DLAEILQKC
SEQ ID NO.: 197:
DLGKKLLEAARAGQDDEVRILMANGADVNAHDTFGFTPLHLAALYGHLE
IVEVLLKNGADVNADDSYGRTPLHLAAMRGHLEIVEVLLKYGADVNAAD
EEGRTPLHLAAKRGHLEIVEVLLKNGADVNAQDKFGKTAFDISIDNGNE
DLAEILQKLC
SEQ ID NO.: 208:
DLGKKLLEAARAGQDDEVRILMANGADVNAHDTFGFTPLHLAALYGHLE
IVEVLLKNGADVNADDSYGATPLHLAAMRGHLEIVEVLLKYGADVNAAD
EEGATPLHLAAKAGHLEIVEVLLKNGADVNAQDKFGKTAFDISIDNGNE
DLAEILQKLC

### Example 3. In Silico Design of CKS-Based Carrier Building Blocks

Cyclin-dependent kinase subunit 1 (CKS1, Gene ID: 983) was selected as starting point for developing the CKS1-based carrier building block. In particular, the polypeptide of SEQ ID NO.: 190 was selected as the building-block carrier precursor. FIG. 3 shows the amino acid sequence of the CKS1-building block precursor. Using MAESTRO, residues in the building block precursor with a Solvent-Accessible Surface Area (SASA) greater than or equal to 27 Å² (square angstrom) were considered to be solvent-accessible and further considered to calculate stability of a mutation to a cysteine. As a result, the following potential well solvent-accessible residues were selected in SEQ ID NO.: 190 as potential positions for point mutations with cysteines, to generate conjugation sites or attachment points in the protein-based carrier building block:
1-4, 6-7, 9-20, 22, 25-27, 29-30, 32-36, 38-41, 43-44, 46, 48, 50-52, 54, 56-64 and 69-78.

Stability (ΔG in solvent) of each mutant was calculated using MAESTRO, see, e.g., Laimer J. et al, “MAESTRO—multi agent stability prediction upon point mutations”, BMC Bioinformatics, 2015, 16:116, for further details. Destabilizing cysteine mutations were not further considered as potential positions for conjugation sites or attachment points. Based on the stability data, the following potential positions in SEQ ID NO.: 190 were further selected as potential solvent-accessible positions based on the in-silico predictions:
1, 4, 9, 10, 11, 12, 13, 22, 25, 29, 33, 51, 57 and an engineered C-terminal Cys.

As a result, the following protein-based carrier building blocks comprising three or six attachment points or conjugation sites which are the —SH group in the side chain of three or six cysteines located at solvent-accessible positions were designed:
a) 23001, corresponding to SEQ ID NO.: 99, which comprises the following point mutations in SEQ ID NO.: 190, at the following positions: K10C, D13C, and a C-terminal Cys;
b) 23002, corresponding to SEQ ID NO.: 100, which comprises the following point mutations in SEQ ID NO.: 190, at the following positions: K10C, D12C, M22C, Q51C;
c) 23003, corresponding to SEQ ID NO.: 101, which comprises the following point mutations in SEQ ID NO.: 190, at the following positions: Y11C, Q51C and a C-terminal Cys;
d) 23004, corresponding to SEQ ID NO.: 102, which comprises the following point mutations in SEQ ID NO.: 190, at the following positions: D13C, K33C, and a C-terminal Cys;
e) 23005, corresponding to SEQ ID NO.: 10³, which comprises the following point mutations in SEQ ID NO.: 190, at the following positions: Y11C, K33C, Q51C, and a C-terminal Cys;
f) 26001, corresponding to SEQ ID NO.: 104, which comprises the following point mutations in SEQ ID NO.: 190, at the following positions: D9C, D13C, M22C, K33C, Q51C, and a C-terminal Cys; and
g) 26002, corresponding to SEQ ID NO.: 105, which comprises the following point mutations in SEQ ID NO.: 190, at the following positions: D9C, D13C, K33C, Q51C, M57C, and a C-terminal Cys.

As control, the polypeptides of SEQ ID NOs.: 201
(SHKQIYYSDKCDDEEFEYRHVMLPKDIAKLVPKTHLMSESEWRNLGVQQSQGWVHYMIHEPEPHILLFR RPLPKKPKK) and 202
(SHKQIYYSDKYDCEEFEYRHVMLPKDIAKLVPKTHLMSESEWRNLGVQQSQGWVHYMIHEPEPHILLFR RPLP) were designed.

### Example 4. Construction, Expression and Purification of Molecules Comprising a Protein-Based Building Block, a 15GS Linker and a HLE Moiety

In addition to the protein-based building blocks as described in Examples 1-3, it was contemplated that the molecules of this example would also comprise a HLE moiety linked to the protein-based building block through a 15GS linker (as defined in SEQ ID NO.: 163). The HLE moiety chosen was Alb23002 (SEQ ID NO.: 63) or its E1D variant (Alb23002 (E1D), SEQ ID NO.: 106). Both Alb23002 and Alb23002 (E1D) are ISVDs which bind to human serum albumin, as explained above in this description. Both polypeptides (the protein-based carrier building block and the HLE moiety) were designed to be linked through a 15GS linker, as defined in SEQ ID NO.: 163. The design of the molecule was as follows:
(N-terminal) HLE-15GS linker-building block (C-terminal)

To produce/the above-described molecules comprising a HLE moiety, a 15GS linker and the protein-based building block, Komagataella phaffii (Pichia pastoris) was used for the expression and purification. This organism is a well-known expression system to the skilled person, as for instance described in Bustos, C. et al., “Advances in cell engineering of the Komagataella phaffii platform for recombinant protein production”, Metabolites, 2022, 12, 346. E. coli may also be used, as described, e.g., in Correa A and Oppezzo P., “Overcoming the solubility problem in E. coli: available approaches for recombinant protein production”, Methods Mol Biol., 2015; 1258:27-44.

In order to facilitate purification, constructs were made with at least one Protein A-binding building block (as the HLE moiety) and expressed via Komagataella phaffii. To this end, an Alb23002 building-block (SEQ ID NO.: 106 or SEQ ID NO.: 63) was genetically fused to the selected protein-based carrier building-block via an 15GS linker (SEQ ID NO.: 163) as described above, thus also rendering the constructs with higher in vivo life extension. During fermentation, cysteamine was added to cap any free thiol of the engineered cysteines and render a homogeneous product for down-stream processing (DSP).

The sequences of the protein-based carrier building block used are summarized in Tables 4-7 (SEQ ID NO.: 80-105, 175, 199, 208, 222-224), and described in detail in Examples 1-3 and 19. The sequences of the multivalent molecules comprising the HLE moiety, linker and protein-based building block, expressed in Komagataella phaffii and purified as described above are summarized in Tables 11-13 (SEQ ID NO.: 107-127, 170-174, 176 or 200).

In particular, fusion constructs, with secretion signal, were expressed via Komagataella phaffii, as described above. Production of ISVDs in lower eukaryotic hosts such as Komagataella phaffii has been described by Frenken et al. (“Isolation of antigen specific llama VHH antibody fragments and their high level secretion by Saccharomyces cerevisiae”, J. Biotechnol., 2000, 78:11-21) and in WO 94/25591, WO 2010/125187, WO 2012/056000, WO 2012/152823 and WO 2017/137579. The contents of these applications are explicitly referred to in the connection with general culturing techniques and methods, including suitable media and conditions. The skilled person can also devise suitable genetic constructs for expression of domains in host cells on the basis of common general knowledge.

Following high cell density fermentation, fusion products were separated from the cells via centrifugation and the molecules were purified from the spent medium. To promote a homogeneous product, a minimum of 10 mM cysteamine (30070, Sigma-Aldrich) was added at the end of the induction phase of the fermentation to cap any free thiol of the engineered cysteines and render a homogeneous product for DSP.

Following 0.22 μm filtration, the thiol-capped product was captured from spent medium via Protein A chromatography and separated on a sizing column and/or via ion exchange (all methods are generally applied during protein purification, see, e.g., Remans, K. et al., “Protein purification strategies must consider downstream applications and individual biological characteristics”, Microb Cell Fact, 2022, 21 (52) or Rathore AS. et al., “Recent developments in chromatographic purification of biopharmaceuticals” Biotechnol Lett., 2018, 40 (6): 895-905). General chromatography conditions for Protein A were applied, and the proteins of interest were eluted with 100 mM Glycine pH 2.5 and neutralised using 1 M Tris pH8. The reduced material was formulated in D-PBS+0.1 mM TCEP to keep the reduced state and allow direct conjugation upon thawing.

### Example 5. Conjugation of Cargo Molecules to the Protein-Based Carrier Building Block with Three Conjugation Sites

### 5.1 Conjugation of the APN-Maleimide Linker

Conjugation experiments were performed using protein-based building block as defined in SEQ ID NO.: 80 (13001, see Table 4), comprised in a molecule (SEQ ID NO.: 107) which further comprises the Alb23002 HLE moiety (Alb23002 (E1D), SEQ ID NO.: 106, see Table 8) and a 15GS linker (SEQ ID NO.: 163, see Table A-1), as defined above, see also, e.g., Table 11. The protein-based carrier building block as defined in SEQ ID NO.: 80 comprises three attachment points or conjugation sites which are the —SH groups of three cysteines located at positions 43, 100f and 105 (according to Kabat numbering). The cysteines have been introduced in the original molecule (SEQ ID NO.: 179) as point mutations in the above-mentioned positions (according to Kabat numbering): K43C, D100fC and R105C. In addition, the glutamine at hallmark position 108 has been replaced by a leucine (Q108L).

The engineered cysteines present in the ISVD-based building block (SEQ ID NO.: 80)-comprising molecule (SEQ ID NO.: 107) or in the control molecule (SEQ ID NO.: 176, which protein-based building block (SEQ ID NO.: 175) comprises a C-terminal Cys with a —SH in its side chain which is the attachment point of this building block) were reduced and/or uncapped using 10 mM in DTT PBS, after which DTT was separated from the molecule solution (comprising the ISVD-building block with reduced engineered cysteines) via HiPrep™ 26/10 Desalting (Cytiva 17-5087-01) or Size Exclusion Chromatography (SEC) on Superdex® Increase 75 10/300 GL (Cytiva 29-1487-21), equilibrated in D-PBS with 0.1 mM TCEP (20490 Thermo Scientific™ Pierce™). This material can be used directly for conjugations or frozen at −20° C. to be used later (TCEP will prevent reoxidation of the free thiols).

For an assessment of the conjugability of the engineered cysteines (with the side chain —SH groups as attachment points or conjugation sites) present in the building block, any small, maleimide-activated ligand can be used. For instance, the APN-Maleimide linker (806536, Sigma-Aldrich) or N-Maleoyl-β-alanine (394815, Sigma-Aldrich, also referred to as maleimide-Alanine) can be used to this end. The excess of unconjugated ligand was removed via SEC or desalting, and the conjugation product was analysed via Mass Spectrometry (MS). LC-MS was carried out using a Q Exactive™ Plus Hybrid Quadrupole-Orbitrap™ Mass Spectrometer and a Vanquish Flex UHPLC (both Thermo Scientific®) with an online Waters MassPREP™ Micro Desalting Columns (2.1×5 mm, P/N. 186004032).

Alternatively, an early assessment of the conjugability of the engineered cysteines could be carried out making use of tris(2-carboxyethyl)phosphine (TCEP) as reductant. TCEP does not need to be removed during maleimide based conjugations. Typically, 0.1 mM TCEP in D-PBS is used. A 5-fold or higher excess of maleimide-Alanine vs engineered cysteines was used to promote full conjugation of available free thiols. The crude mixture was not separated via SEC, but instead directly analysed via LC-MS to assess the number of cysteines with (near) full conjugation.

In the present example, an APN-maleimide ‘bifunctional’ linker (Formula I, Sigma-Aldrich #806536) was used to connect different cargos to the protein-based building block comprised in the molecule. This linker allows for conjugation twice, via cysteine-based chemistry. Both APN and maleimide couple to free thiols, albeit at different speed. An example is shown in FIG. 4.

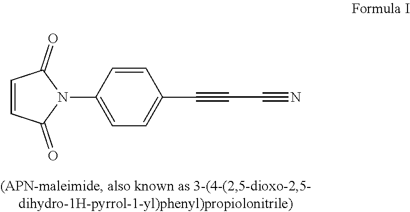 [SMILES: N#CC#Cc1ccc(N2C(=O)C=CC2=O)cc1]

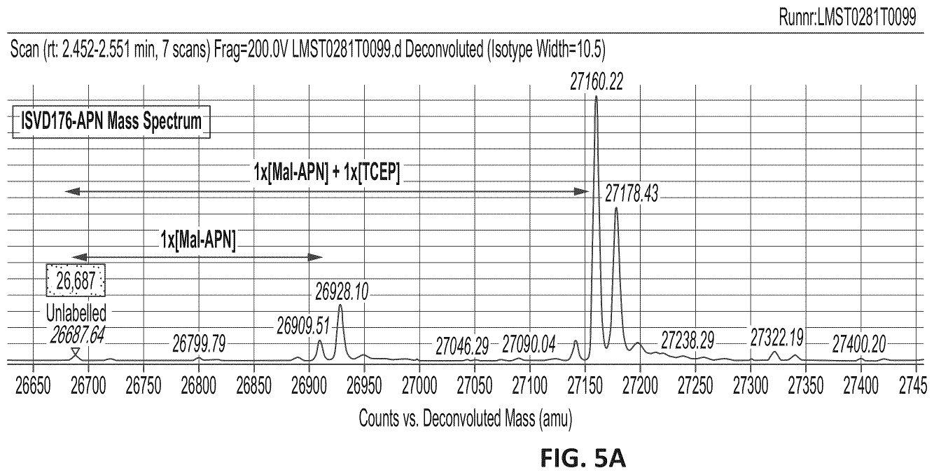

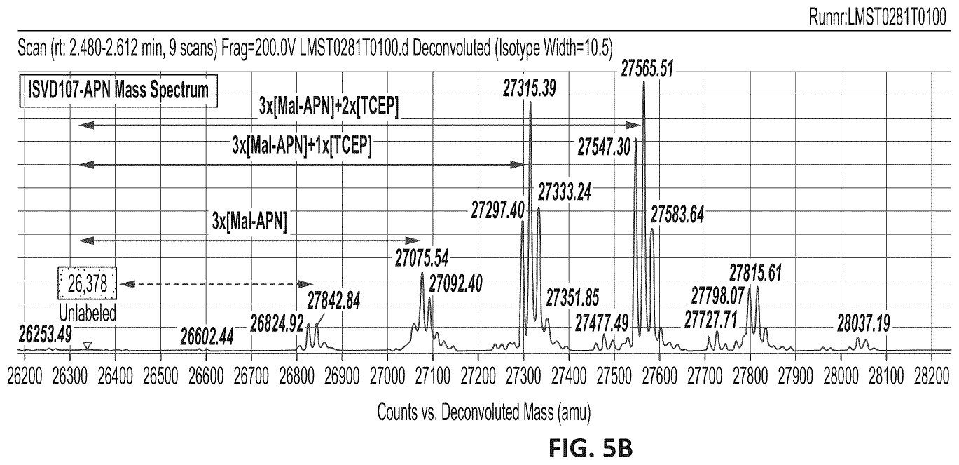

For maleimide-based conjugation, generic conditions were applied (if not otherwise indicated). 5× molar mass excess of APN-maleimide in DSMO was added to 1×ISVD-based building block (SEQ ID NO.: 80)-comprising molecule (SEQ ID NO.: 107). As a control, SEQ ID NO.: 176 was used (same molecule but without the cysteine point mutations as described above and with a C-terminal Cys (-GGC)). The ISVD-based building block-comprising molecule (SEQ ID NO.: 107) and control molecule (SEQ ID NO.: 176) were formulated in D-PBS+0.1 mM TCEP, to prevent thiol oxidation. The mixture was incubated for 10 min at room temperature (RT), head over head rotating. Afterwards, the excess APN-maleimide was removed via size exclusion chromatography (SEC), and the resulting molecules were formulated in D-PBS. At this point a sample was taken for mass spectrometry analysis, to check the conjugation efficiency on the engineered cysteine residues. LC-MS was done using a Q Exactive™ Plus Hybrid Quadrupole-Orbitrap™ Mass Spectrometer and a Vanquish Flex UHPLC (both Thermo Scientific®) with an on line Waters MassPREP™ Micro Desalting Columns (2.1×5 mm, P/N. 186004032). As shown in FIG. 5, additions of 222 Da (corresponding to the APN-maleimide linker addition) and 286 Da (corresponding to TCEP, used to keep the free thiols reduced) did indicate efficient conjugation onto both molecules (SEQ ID NO.: 107 and control SEQ ID NO.: 176). The presence of two or three peaks for each of the molecules simply reflect the mass of the molecule on its own (with the APN-maleimide linker) or the molecule plus one or two molecules of TCEP, as also indicated in the figure. On the control, the added mass clearly corresponded to linker conjugation (FIG. 5A). On all 3 positions of SEQ ID NO.: 107 (43, 100f and 105, according to Kabat), the linker was present as the corresponding masses were detected (FIG. 5B).

### 5.2 Conjugation of Anti-DR5 ISVDs

The molecules described above in 5.1 (SEQ ID NO.: 107 and 176) with the APN-maleimide linkers were further used to conjugate the cargos, which in this specific example were DR5-GGC ISVDs:
a) Mono DR5-GGC (SEQ ID NO.: 177); and
b) Trivalent DR5-GGC (SEQ ID NO.: 178).

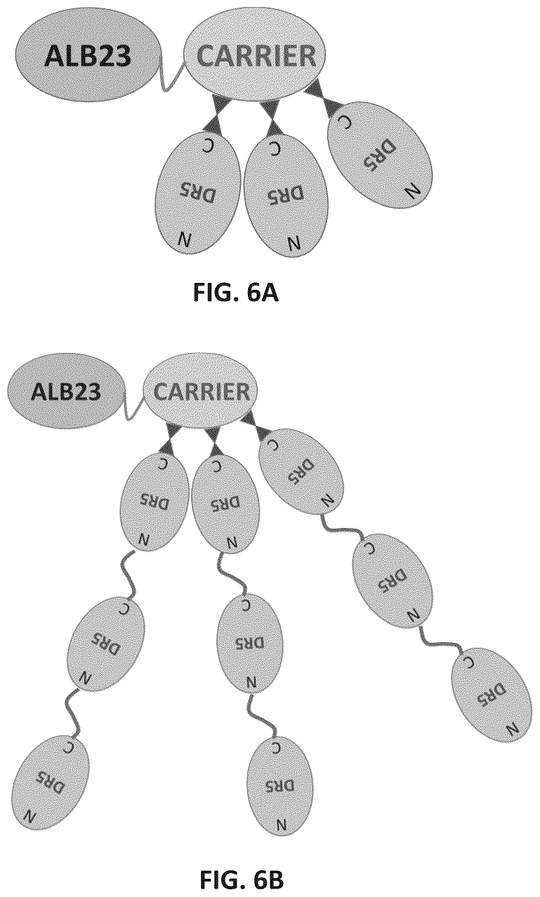

In both cases, the cargos comprise ISVDs specific to DR5. The monovalent cargo comprises a single DR5 ISVD, whereas the trivalent cargo comprises three DR5 ISVDs, see FIG. 6.

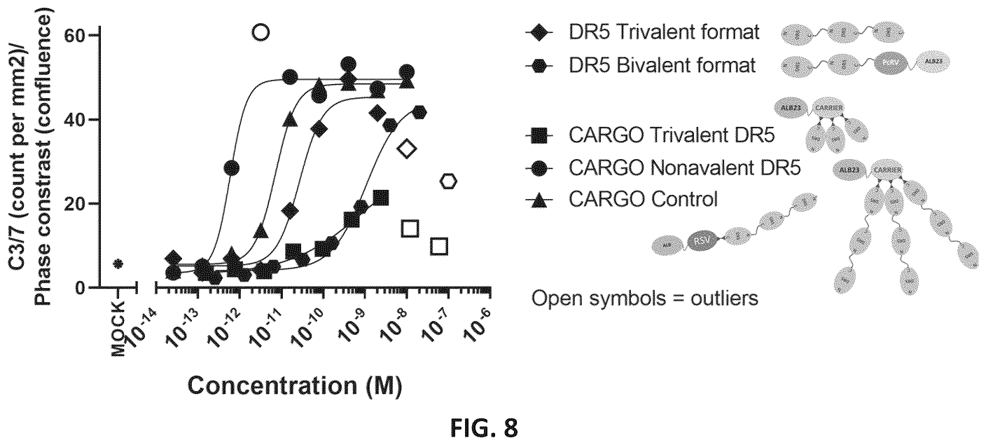

Both mono- and trivalent DR5-GGC cargos (SEQ ID NO.: 177 and 178), see above, were conjugated to the molecule comprising SEQ ID NO.: 107 and the APN-maleimide linkers. To the control molecule (SEQ ID NO.: 176 and the APN-maleimide linker), only a single trivalent DR5-GGC was conjugated. See FIG. 8 for the graphic representation of the molecules:
a) CARGO Trivalent DR5: molecule as defined in SEQ ID NO.: 107, with three attachment points which are the —SH in the side chain of the Cys at positions 43, 100f and 105. The APN-maleimide linker is attached to these conjugation sites on one side and, on the other side, the linker has the cargo (monovalent DR5-GGC (SEQ ID NO.: 177)) attached to it. Hence, the cargos are attached to the three conjugation sites via APN-maleimide linkers;
b) CARGO Nonavalent DR5: molecule as defined in SEQ ID NO.: 107, with three attachment points which are the —SH in the side chain of the Cys at positions 43, 100f and 105. The APN-maleimide linker is attached to these conjugation sites on one side and, on the other side, the linker has the cargo (trivalent DR5-GGC (SEQ ID NO.: 178)) attached to it. Hence, the cargos are attached to the three conjugation sites via APN-maleimide linkers; and
c) CARGO control: molecule as defined in SEQ ID NO.: 176, with one attachment point which is the —SH in the side chain of the C-terminal Cys. The APN-maleimide linker is attached to this conjugation site on one side and, on the other side, the linker has the cargo (trivalent DR5-GGC (SEQ ID NO.: 178)) attached to it. Hence, the cargo is attached to the conjugation site via an APN-maleimide linker.

In particular, the molecules comprising the Mal-APN linkers were first separated from excess free Mal-APN linker via SEC equilibrated in D-PBS+0.1 mM TCEP and further used to conjugate the cargo (DR5-GGC ISVDs). A 2× molar excess of reduced DR5-GGC in D-PBS vs APN-Mal-molecule conjugate was added and incubated for 2 h at RT, in the presence of 0.1 mM TCEP, followed by overnight (ON) incubation at 4° C. in D-PBS. The following conjugations were performed: the APN-Maleimide linker was first conjugated to the molecules (SEQ ID NO.: 107 and 176), as described above. In a second step, mono and trivalent DR5-GGC (SEQ ID NO.: 177 and 178) were conjugated onto the second functionality of the APN-Maleimide linker.

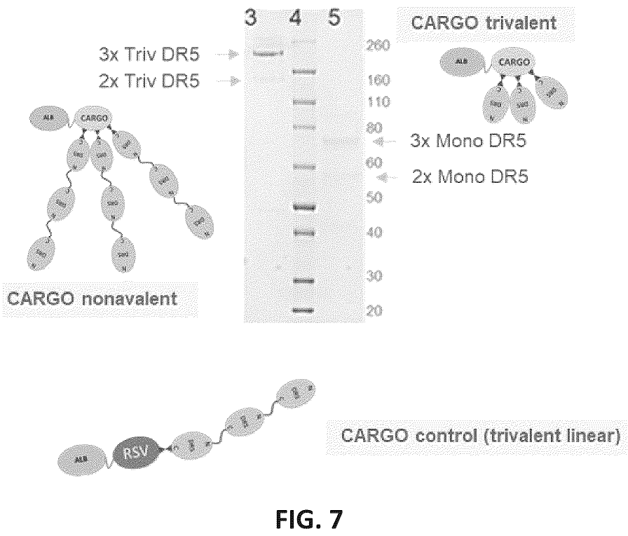

Hence, the following molecules were successfully obtained, see also FIGS. 7 and 8:
a) “CARGO nonavalent”: This molecule comprises a HLE moiety (“ALB” or “Alb23”, SEQ ID NO.: 106), an ISVD-based carrier building block (“CARGO” or “CARRIER”, SEQ ID NO.: 107) with three —SH conjugation sites (at positions 43, 100f and 105, according to Kabat) and three cargos attached via an APN-maleimide linker to the three conjugation sites, wherein each cargo comprises three DR5 ISVDs. Hence, the “CARGO nonavalent” molecule comprises a total of nine DR5 ISVDs;
b) “CARGO trivalent”: This molecule comprises a HLE moiety (“ALB” or “Alb23”, SEQ ID NO.: 106), an ISVD-based carrier building block (“CARGO” or “CARRIER”, SEQ ID NO.: 107) with three —SH conjugation sites (at positions 43, 100f and 105, according to Kabat) and three cargos attached via an APN-maleimide linker to the three conjugation sites, wherein each cargo comprises one DR5 ISVD. Hence, the “CARGO trivalent” molecule comprises a total of three DR5 ISVDs; and
c) “CARGO control” (trivalent linear): This is the control molecule and comprises a HLE moiety (“ALB”, SEQ ID NO.: 106), a control carrier building block (“RSV”, SEQ ID NO.: 176), comprising a C-terminal Cys with a side-chain —SH, to which a molecule comprising three DR5 ISVDs is attached via an APN-maleimide linker. Hence, the “CARGO control (trivalent linear)” molecule comprises a total of three DR5 ISVDs linearly disposed at the C-terminal of the molecule.

As further controls, the following molecules were synthesized (see also FIG. 8):
d) “DR5 Trivalent format” consists of the trivalent DR5-GGC molecule as defined in SEQ ID NO.: 178; and
e) “DR5 Bivalent format” (SEQ ID NO.: 207) consists of two DR5 ISVDs (DR5004E06 (E1D, Q108L) and DR5004E06 (Q108L)) which have been modified as to not bind to their target, linked to each other through a 9GS linker (SEQ ID NO.: 161). These two “non-functional” ISVDs are linked to a protein-based building block called A0095013F07 (A14P, N27L, E41P, R64K, S68T, T74S, D76N, K83R, K105Q), also referred to in FIG. 8 as “PcRV”, through a 35GS linker (SEQ ID NO.: 168). Furthermore, the “PcRV” protein-based building block is linked to an albumin-binding ISVD (SEQ ID NO.: 63) (“ALB 23” in FIG. 8) through another 35GS linker (SEQ ID NO.: 168). Finally, the molecule comprises a C-terminal Ala:
   DR5004E06 (E1D,Q108L)-9GS-DR5004E06 (Q108L)-35GS-A0095013F07 (A14P,N27L,E41P,R64K,S68T,T74S,D76N,K83R,K105Q)-35GS-ALB23002-A,
   SEQ ID NO.: 207:

DVQLVESGGGSVQAGDSLRLSCAASGRTFGSIRVGWFRQTPGKEREFVA
AINRNDGTTYYADSVKGRFTISRDNAKNTVYMQMASLKPEDTAVYYCAA
GLQYNRSADRVPVGAVYWGQGTLVTVSSGGGGSGGGSEVQLVESGGGSV
QAGDSLRLSCAASGRTFGSIRVGWFRQTPGKEREFVAAINRNDGTTYYA
DSVKGRFTISRDNAKNTVYMQMASLKPEDTAVYYCAAGLQYNRSADRVP
VGAVYWGQGTLVTVSSGGGGSGGGGSGGGGSGGGGSGGGGSGGGGSGGG
GSEVQLVESGGGLVQPGGSLRLSCAASGLTFSTNPMYWYRQAPGKQREL
VASISSRGITNYADSVKGRFTISRDNSKNTVYLQMNSLRPEDTAVYYCR
LASLSSGTVYWGQGTLVTVSSGGGGSGGGGSGGGGSGGGGGGGGSGGGG
SGGGGSEVQLVESGGGVVQPGGSLRLSCAASGFTFRSFGMSWVRQAPGK
GPEWVSSISGSGSDTLYADSVKGRFTISRDNSKNTLYLQMNSLRPEDTA
LYYCTIGGSLSRSSQGTLVTVSSA

The conjugation products (molecules (SEQ ID NO.: 107 and 176) with the APN-maleimide linkers and DR5 ISVDs cargos) were size separated into D-PBS via SEC and analyzed via SDS-PAGE. From the PAGE analysis (FIG. 7), the 3× conjugation status is clearly visible in both types of molecules. The control molecules as defined above (not shown in the PAGE analysis) was also purified in parallel.

Next, a potency (apoptosis) assay based on DR5 clustering (Caspase 3/7 assay) was performed. Colo205 cells were plated at 15,000 cells/well. Medium containing the indicated concentrations of DR5 agonists and controls (see also FIG. 8) were added, followed by 5 UM CellEvent™ Caspase-3/7 Green Detection Reagent (Essen Biosciences, cat #4440) according to manufacturer's instructions. Real-time data acquisition and analysis was performed using Incucyte® ZOOM (Essen biosciences). Caspase 3/7 signal was normalized for cell confluency at 24 h timepoint. The results are shown in FIG. 8. From the results shown in this figure, it is clear that molecules comprising three trivalent DR5-GGC (i.e., the “CARGO Nonavalent” molecule) has higher activity than the control (“CARGO control”), indicating the successful conjugation and desired activity. “CARGO Trivalent” (molecules comprising three monovalent DR5-GGC) appears to be less potent than the “CARGO control”. From these results it can be inferred that the APN-maleimide linker may be too short and/or too constraint to have an optimal DR5 target binding and engagement.

However, such a restricted target engagement could be useful when a too high toxicity is obtained with the linear format or lower binding would be preferred, for instance, only binding to high-expressing cells. In addition, this short linker might be suited for other targets with smaller geometry. Hence, the length of the linker can be optimized based on the nature of the cargo, the position of the attachment points and the desired activity of the molecule.

### 5.3 Conjugation of Polyethylene Glycol

Further conjugations were performed on the —SH attachment points or conjugation sites in the ISVD-based carrier building block (SEQ ID NO.: 80), comprised in the molecule defined by SEQ ID NO.: 107. As described in Example 5.1, the protein-based carrier building block as defined in SEQ ID NO.: 80 comprises three attachment points or conjugation sites which are the —SH groups of three cysteines located at positions 43, 100f and 105 (according to Kabat numbering). The cysteines have been introduced in the original molecule (SEQ ID NO.: 179) as point mutations in the above-mentioned positions (according to Kabat numbering): K43C, D100fC and R105C. In addition, the glutamine at position 108 has been replaced by a leucine (Q108L).

In the present case, the following conjugations were performed:
a) N-ethylmaleimide (Formula II);
b) Maleimido-PEG12-acid (PubChem CID 68757103, UPAC name 3-[2-[2-[2-[2-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]ethoxy]ethoxy]ethoxy]ethoxy]propanoic acid, also known as “Mal-amido-PEG12-acid”); and
c) Resiquimod-maleimido PEG24, a cargo which comprises Resiquimod (PubChem CID 159603, Formula (III), an imidazoquinolinamine and Toll-like receptor (TLR) agonist with potential immune response modifying activity) and maleimido PEG24, as described above. Formula (IV) shows the Resiquimod-maleimido PEG24 cargo.

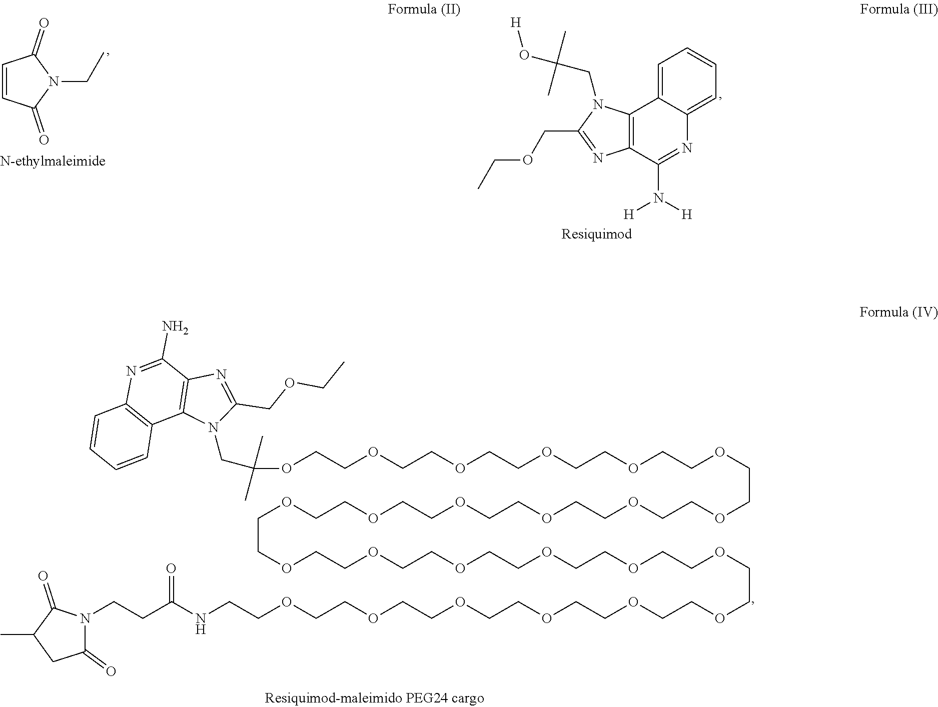

In all cases, in a first step, 10 mg/ml of the molecule comprising the HLE moiety and the protein-based carrier building block (SEQ ID NO.: 107) was treated with 10 mM DTT for 1 h at RT, to set free the attachment points (—SH) in a reduced state after which the DTT was removed via size exclusion chromatography. Conjugations with the above cargos were carried out in D-PBS/DMSO (96:4 to 80:20) pH 7.4 with a 5-10 cargo: carrier ratio. All reactions resulted in more than 95% of conjugation rate (HPLC). Mass spectrometry analysis demonstrated the presence of the different cargos attached to the molecule.

### Example 6. Conjugation of Cargo Molecules to the Protein-Based Carrier Building Block with Six Conjugation Sites

Molecules comprising one ISVD-based building block with six conjugation sites or attachment points were produced as described in Examples 1 and 4. In particular, the protein-based building block as defined in SEQ ID NO.: 86 (see Table 5), comprised in a molecule which further comprises an HLE moiety (Alb-23002, SEQ ID NO.: 106) and 15GS linker (SEQ ID NO.: 163), as defined above (molecule T028100078, SEQ ID NO.: 113, see Table 11) was used. This protein-based carrier building block comprises six attachment points or conjugation sites which are the —SH groups of six cysteines located at positions 19, 44, 65, 70, 82b and 112 (according to Kabat numbering). The cysteines have been introduced in the original molecule (SEQ ID NO.: 179) as point mutations in the above-mentioned positions (according to Kabat numbering): S19C, E44C, G65C, S70C, S82bC, and S112C. In addition, the glutamine at position 108 has been replaced by a leucine (Q108L).

Once the polypeptide with SEQ ID NO.: 113 was expressed (see Example 4), the material was capped with cysteamine at the end of fermentation and purified via Protein A. The reduced material was formulated in D-PBS+0.1 mM TCEP to keep the reduced state and allow direct conjugation upon thawing.

In order to assess whether the selected attachment points would be useful for cargo conjugation, a “model cargo” (a maleimide-modified alanine, N-Maleoyl-β-alanine (mal-Ala), in this case) was used. Conjugation with N-Maleoyl-β-alanine was performed and mass spectrometry analysis indicated that the molecule was fully conjugated, indicating that all 6 cysteines are available for conjugation. In view of these results, this molecule (SEQ ID NO.: 113) was selected to attach or conjugate cargos on it. In particular, the aim was (i) to conjugate 5 kDa PEG molecules in 50% of the conjugation sites (i.e., in 3 conjugation sites of the molecule) and to conjugate a cargo comprising Cryptophycin (see, e.g., Verma V A., et al., “The cryptophycins as potent payloads for antibody drug conjugates”, Bioorg Med Chem Lett. 2015, 25 (4): 864-8) in the remaining 50% of the conjugation sites (i.e., in the remaining 3 conjugation sites in the molecule, “DOL3 molecule”); and (ii) to conjugate 5 kDa PEG in the 100% of the conjugation sites (i.e., in the six conjugation sites, “DOL6 molecule”).

The first step was carried out to conjugate 5 kDa PEG onto 50% of the conjugation sites of molecule with SEQ ID NO.: 113, in order to achieve an average occupation of the 50% of the conjugation sites (an average of 3 conjugation sites occupied by 5 kDa PEG per molecule), “DOL3 molecule” or “DOL3 material”. For this, a partial/stochastic labeling was carried out using a 1.2 excess of maleimide PEG vs the three free thiols that were to be conjugated (this corresponds to a 0.6 5 kDa PEG ratio vs 6 cysteines). Following the conjugation aiming at 50% occupation of the attachment points, using this 0.6 ratio vs the 6 cysteines, half of the material was withdrawn for further conjugation with the Cryptophycin-comprising cargo. Of this material, a small sample was taken. From this sample, the remaining free thiols were blocked using maleimide alanine for Mass Spec analysis of the conjugation status. The rest of the material was frozen. Mass Spec analysis did show that an average of molecules with 50% of the conjugation sites occupied with 5 kDa PEG was present. LC-MS was done using a Q Exactive™ Plus Hybrid Quadrupole-Orbitrap™ Mass Spectrometer and a Vanquish Flex UHPLC (both Thermo Scientific®) with an online Waters MassPREP™ Micro Desalting Columns (2.1×5 mm, P/N. 186004032).

The conjugation of the remainder half of the material was continued overnight with excess 5 kDa PEG-maleimide aiming for complete PEGylation (5 kDa PEG in the 100% of the conjugation sites, the “DOL6 molecule”). Both preps were size separated to recover the “DOL3” and “DOL6” fraction, confirming that the conjugation has taken place.

The remaining DOL3 material with 3 free thiols (and 3 thiols occupied by 5 kDa PEG, see above) was formulated in PBS+0.1 mM TCEP to be further conjugated with the Cryptophycin-comprising cargo, aiming for a molecule in which 50% of the conjugation sites (i.e., 3 conjugation sites, on average) are occupied by 5 kDa PEG and the remaining 50% of the conjugation sites (i.e., the remaining 3 conjugation sites, on average) are occupied by a the Cryptophycin-comprising cargo, via a maleimide linker. The maleimide Cryptophycin-comprising cargo conjugation was carried out as described above and the resulting conjugates were analysed via SDS-PAGE and SEC.

### Example 7. siRNA Conjugation on Molecule Comprising ISVD-Based Carrier Building Block

The molecule comprising ISVD-based carrier building block was expressed and purified as described in Example 4. This molecule is described on Table 11 (T028100070, SEQ ID NO.: 108) and it comprises an HLE moiety (SEQ ID NO.: 106), a 15GS linker (SEQ ID NO.: 163) and an ISVD-based building block (SEQ ID NO.: 81). The protein-based carrier building block comprised in this molecule has three conjugation sites or attachment points which are the —SH groups of Cys present at the following positions (according to Kabat numbering): 43, 75 and 100a. The cysteines have been introduced in the original molecule (SEQ ID NO.: 179) as point mutations in the above-mentioned positions (according to Kabat numbering): K43C, K75C and G100aC. In addition, the glutamine at hallmark position 108 has been replaced by a leucine (Q108L).

For siRNA conjugation, siRNA (MW 15884.7 Da) was activated with the heterobifunctional linker succinimidyl 4-[N-maleimidomethyl] cyclohexane-1-carboxylate (SMCC) to introduce a maleimide group for conjugation onto the attachment points.

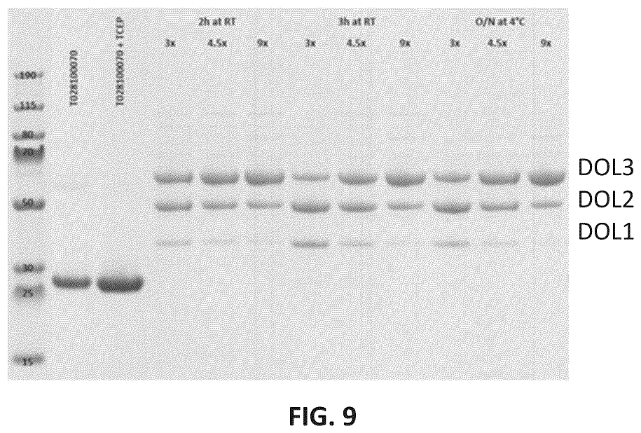

The molecule described in SEQ ID NO.: 108 was reduced using 50× molar excess of TCEP during 3.5 h. Then 10 μg fractions of this material were incubated with 3×, 4.5× of 9× molar excess siRNA for 2 h, 3 h at room temperature (RT) or overnight at 4° C. Samples corresponding to 2 μg of molecule described in SEQ ID NO.: 108 were analysed via SDS-PAGE and stained with Coomassie Brilliant Blue (CBB). See FIG. 9. The results clearly indicate and efficient conjugation of siRNA onto the molecule described in SEQ ID NO.: 108, even at a 3× excess of cargo vs available conjugation sites (9× molar excess) at all conditions tested. In FIG. 9, “DOL3” refers to the molecule described in SEQ ID NO.: 108 with three siRNA molecules attached to the three conjugation sites. “DOL2” refers to the molecule described in SEQ ID NO.: 108 with two siRNA molecules attached to two conjugation sites and “DOL1” refers to the molecule described in SEQ ID NO.: 108 with one siRNA molecules attached to one conjugation site. “T028100070” refers to the molecule described in SEQ ID NO.: 108 on its own, and “T028100070+TCEP” refers to the molecule described in SEQ ID NO.: 108 in its reduced form with TCEP.

### Example 8. Combination of PEGylation and Radiolabeling of the Molecule Described in SEQ ID NO.: 108

The molecule comprising ISVD-based carrier building block was expressed and purified as described in Example 4. This molecule is described on Table 11 (T028100070, SEQ ID NO.: 108) and it comprises an HLE moiety (“Alb23002”, SEQ ID NO.: 106), a 15GS linker (SEQ ID NO.: 163) and an ISVD-based building block (SEQ ID NO.: 81). The protein-based carrier building block comprised in this molecule has three conjugation sites or attachment points which are the —SH groups of Cys present at the following positions (according to Kabat numbering): 43, 75 and 100a. The cysteines have been introduced in the original molecule (SEQ ID NO.: 179) as point mutations in the above-mentioned positions (according to Kabat numbering): K43C, K75C and G100aC. In addition, the glutamine at hallmark position 108 has been replaced by a leucine (Q108L).

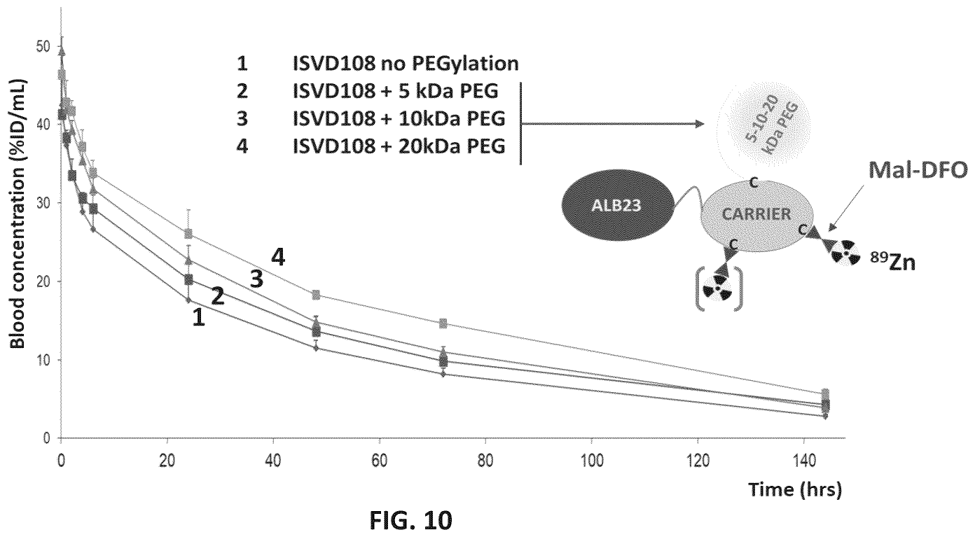

The molecule described in SEQ ID NO.: 108 was reduced using 50× molar excess of TCEP during 3.5 h. Then, the molecule was PEGylated with PEG molecules of different length (5-10-20 kDa maleimide PEG). The aim was to obtain molecules with 1 (DOL1) conjugation sites occupied by the PEG molecules (“ISVD108+5 kDa PEG”, “ISVD108+10 kDa PEG” and “ISVD108+20 kDa PEG” molecules in FIG. 10). To obtain this DOL1 molecule, the reactions were carried out stochastically with a 1.2 molar ratio PEG-molecule (1.2×PEG:1× molecule as defined in SEQ ID NO.: 108) at RT and followed by SEC on Superdex75 to select the DOL1 fraction. In a next step 2-3 eq. of maleimide-desferoxamine (mal-DFO) in 20 mM in DMSO was added and incubated for another 16 h at RT, in order to conjugate two mal-DFO molecules in the remaining conjugation sites of the molecule described in SEQ ID NO.: 108. The resulting conjugation mixture was separated via SEC and QC′d via ESI-MS, confirming the presence of one PEG molecule and two mal-DFO per molecule described in SEQ ID NO.: 108. As a control, molecules without PEGylation were uploaded with mal-DFO, see “ISVD108 no PEGylation”, FIG. 10.

The mal-DFO chelators were uploaded with ⁸⁹Zirconium at RT and free 89Zn was separated via Zeba Spin columns equilibrated in 20 mM citric acid. The resulting 89Zn labelled molecule (see also FIG. 10) was tested in vivo via single dose IV injection (tail vein) at 1 mg/kg in C57BL6 WT mice (4 groups of 3 mice). Blood samples were withdrawn for blood PK at 5 min, 4 h, 24 h, 48 h, 72 h and 144 h post-injection and gamma emission was counted. The results in FIG. 10 show the expected half-life dictated by serum albumin binding of Alb23002 (SEQ ID NO.: 106), which is further aided by PEGylation through size depended enhanced renal clearance.

### Example 9. Conjugation of 5 kDa PEG and Hydrophobic Payloads to a Protein-Based Carrier Building Block with Three or Six Conjugation Sites

The molecules comprising ISVD-based carrier building block were expressed and purified as described in Example 4. These molecules are described on Table 11 (T028100069, T028100070 and T028100078, SEQ ID NOs.: 107, 108 and 113, respectively).

Molecule with SEQ ID NO.: 107 comprises an HLE moiety (“ALB23”, SEQ ID NO.: 106), a 15GS linker (SEQ ID NO.: 163) and an ISVD-based building block (“CARRIER”, SEQ ID NO.: 80). The protein-based carrier building block comprised in this molecule has three conjugation sites or attachment points which are the —SH groups of Cys present at the following positions (according to Kabat numbering): 43, 100f and 105. The cysteines have been introduced in the original molecule (SEQ ID NO.: 179) as point mutations in the above-mentioned positions (according to Kabat numbering): K43C, D100fC and R105C. In addition, the glutamine at hallmark position 108 has been replaced by a leucine (Q108L).

Molecule with SEQ ID NO.: 108 comprises an HLE moiety (“ALB23”, SEQ ID NO.: 106), a 15GS linker (SEQ ID NO.: 163) and an ISVD-based building block (“CARRIER”, SEQ ID NO.: 81). The protein-based carrier building block comprised in this molecule has three conjugation sites or attachment points which are the —SH groups of Cys present at the following positions (according to Kabat numbering): 43, 75 and 100a. The cysteines have been introduced in the original molecule (SEQ ID NO.: 179) as point mutations in the above-mentioned positions (according to Kabat numbering): K43C, K75C and G100aC. In addition, the glutamine at hallmark position 108 has been replaced by a leucine (Q108L).

Molecule with SEQ ID NO.: 113 comprises an HLE moiety (“ALB23”, SEQ ID NO.: 106), a 15GS linker (SEQ ID NO.: 163) and an ISVD-based building block (“CARRIER”, SEQ ID NO.: 86). The protein-based carrier building block comprised in this molecule has six conjugation sites or attachment points which are the —SH groups of Cys present at the following positions (according to Kabat numbering): 19, 44, 65, 70, 82b and 112. The cysteines have been introduced in the original molecule (SEQ ID NO.: 179) as point mutations in the above-mentioned positions (according to Kabat numbering): S19C, E44C, G65C, S70C, S82bC, and S112C. In addition, the glutamine at hallmark position 108 has been replaced by a leucine (Q108L).

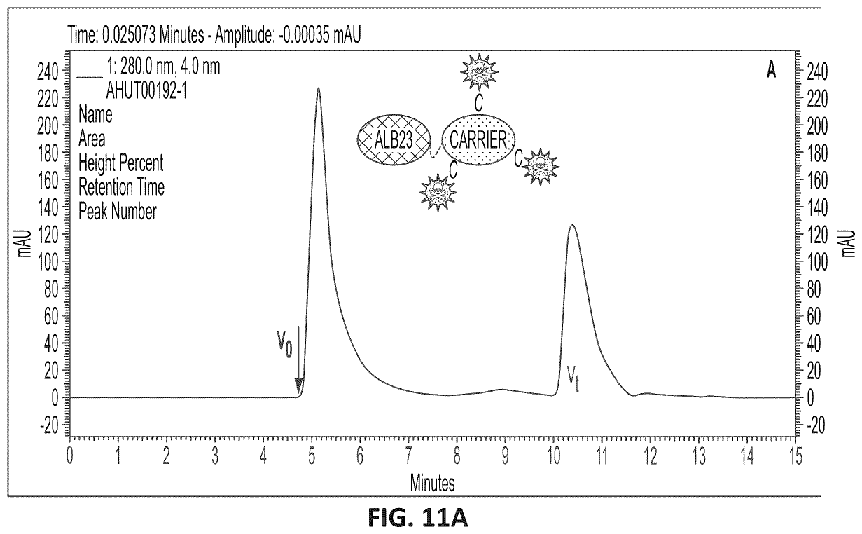

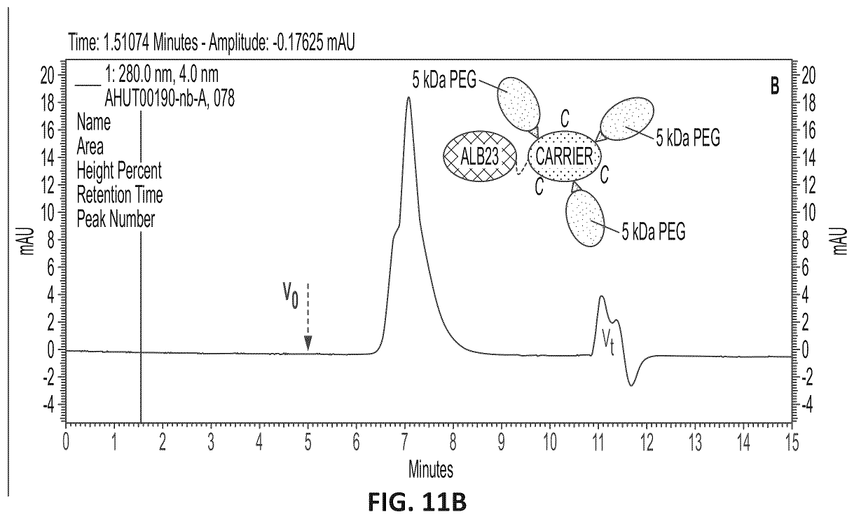

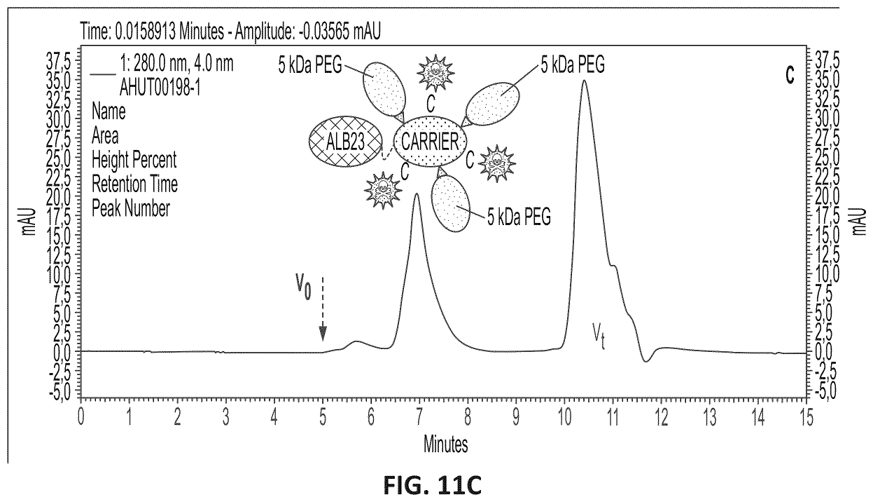

Molecules with SEQ ID NOs.: 107 and 108 both at ≈3 mg/ml (dilution with DPBS+1 mM EDTA) and ≈10% DMA or DMSO were conjugated with a ≈7 equivalents ratio of Mal-Cryptophycin (e.g., Mal-Cryptophycin). Molecule with SEQ ID NO.: 113 at a concentration of 0.28 mg/ml with 1% DMA or DMSO was conjugated with a 10-20 equivalents cargo ratio. Molecules with SEQ ID NOs.: 107 and 108 with hydrophobic payloads conjugated to the three conjugation sites did show precipitation or aggregates on SEC. For instance, as shown in FIG. 11A, molecule with SEQ ID NO.: 108 uploaded with three Mal-Cryptophycin molecules resulted in precipitation and aggregates. The material elutes in exclusion volume of the SEC column (V₀).

The molecule with SEQ ID NO.: 113 was first uploaded either with three 5 kDa PEG molecules (50% of the attachment points) to which three cargos comprising Cryptophycin were added, as described in Example 6 above. FIG. 11B shows that the molecule with SEQ ID NO.: 113 and 50% of the attachment points occupied by 5 kDa PEG molecules did not aggregate or precipitate, as the material shows a retention time as expected. In addition, the molecule with SEQ ID NO.: 113 with 50% of the attachment points occupied by 5 kDa PEG molecules and the other 50% of the attachment points occupied by the cargos comprising Cryptophycin results in soluble material and normal retention time on SEC, see FIG. 11C. A similar observation was done with other hydrophobic cargos, DM4, an antitubulin agent that inhibits cell division, and Resiquimod (data not shown), pointing to the general applicability of this approach. Such a wrapping/shielding of hydrophobic cargos can not only lead to better solubility, but could also shield the toxins in vivo, resulting in a better therapeutical index.

### Example 10. Workflow Example from Design to Conjugability of Engineered Cysteines on a Carrier Comprising Six Conjugation Sites which are the —SH Groups of Six Cys Located at Solvent-Accessible Positions

Further molecules comprising one ISVD-based building block with six conjugation sites or attachment points were designed, produced and purified as described in Examples 1 and 4. In particular, the protein-based building blocks as defined in SEQ ID NOs.: 90-93, comprised in a molecule comprising Alb-230023 HLE moiety (SEQ ID NO.: 63) and 15GS linker (SEQ ID NO.: 163), as defined above (SEQ ID NOs.: 117-120, T028100118, T028100119, T028100120 and T028100121, see Table 11) were selected, based on their expression levels. These protein-based carrier building blocks comprise six attachment points or conjugation sites which are the —SH groups of six cysteines located at the following positions (according to Kabat numbering):
a) Protein-based building block as defined in SEQ ID NO.: 90: 25, 43, 75, 82b, 100a and 112;
b) Protein-based building block as defined in SEQ ID NO.: 91: 25, 43, 75, 100a, 105, and 112;
c) Protein-based building block as defined in SEQ ID NO.: 92: 25, 43, 75, 100a, 105 and a C terminal cysteine;
d) Protein-based building block as defined in SEQ ID NO.: 93: 43, 68, 75, 100a, 105 and a C-terminal cysteine.

The cysteines have been introduced in the original molecule (SEQ ID NO.: 179) as point mutations in the above-mentioned positions (according to Kabat numbering). In addition, the glutamine at position 108 has been replaced by a leucine (Q108L), the leucine at position 11 has been replaced by a valine (L11V) and the valine at position 89 has been replaced by a leucine (V89L).

The molecules were produced at small scale and purified via ProteinA chromatography, as described above. General chromatography conditions were applied, and the molecule fraction was eluted with 100 mM Glycine pH2.5 and neutralised using 1 M Tris pH8.

In order to assess whether the selected attaching points would be useful for cargo conjugation, a “model cargo” (N-Maleoyl-β-alanine (mal-Ala)) was used. The expressed proteins (SEQ ID NOs.: 117-120) were reduced with 10 mM TCEP and after overnight reduction at 4° C. used directly for conjugation. N-Maleoyl-β-alanine (mal-Ala) was added in 10× excess and allowed to conjugate for 3 h at RT. Then the obtained conjugates were size separated to remove excess mal-Ala and analysed via mass spectrometry, as described in detail above in these examples. In all cases, the expected mass for 6 conjugation events was detected, demonstrating that all 6 cysteines are readily available for conjugation (data not shown).

In particular, for molecules with SEQ ID NOs.: 117-119 (T028100118, T028100119 and T028100120) almost only the expected mass for 6 conjugation events was detected, demonstrating that all 6 cysteines are readily available for conjugation. For the molecule with SEQ ID NO.: 120 (T028100121), only minor amounts of 6× mal-Ala were detected, indicating that one cysteine is less accessible for conjugation. Based on comparing the positions of engineered cysteines it appears that the thiol of cysteine at position 68 (Kabat numbering) is not highly reactive, as this position is unique to T028100121 amongst the other molecules tested.

### Example 11. GE11 Peptide Conjugation on Molecules Comprising DARPin-Based Carrier Building Blocks

As described above, EGFR-binding oligopeptide GE11 (YHWYGYTPQNVI, SEQ ID NO.: 212) is a dodecapeptide with excellent EGFR affinity. It actively binds, similar to human EGF or anti-EGFR monoclonal antibody cetuximab, the surface of EGFR-positive tumor cells. In this example, the specific effect of multiple GE11 conjugated to DARPin-derived building blocks in cell targeting and internalization, and the specific internalization of the molecules as described herein (pHAb labeled albumin) in cells expressing EGFR was studied. pHAb Amine Dye is a pH sensor dye that has very low fluorescence at pH>7 and a dramatic increase in fluorescence as the pH of the solution becomes acidic.

### Preparation of the Constructs

The GE11 peptide was synthesized according to standard methods and adapted for conjugation: a small linker (GGSGG, SEQ ID NO.: 213) was added to increase flexibility. The C-terminal residue was amidated (GGSGGYHWYGYTPQNVI-NH₂, SEQ ID NO.: 219). A maleimide group was added at the N-terminus (Maleimide-GGSGGYHWYGYTPQNVI-NH₂).

### Preparation of GE11-Conjugates on DARPin-Comprising Molecules

The GE11 peptide was conjugated to DARPin-comprising molecules. The molecules comprising DARPin-based carrier building block were expressed and purified as described in Example 4.

The peptide was conjugated onto three molecules, all comprising a half-life extending moiety which is an albumin-binding ISVD (SEQ ID NO.: 106, see Table 13) and a protein-based carrier building block which is a DARPin-derived building block, see Table 13: ALB-1C_K27m (SEQ ID NO.: 200), ALB-3C_K27m_w1 (SEQ ID NO.: 173) and ALB-5C_K27m (SEQ ID NO.: 174). All conjugations were purified via SEC and the correct conjugation and number of GE-11 peptides per molecule was confirmed via ESI-MS.

### Functional Characterization of Molecules Comprising DARPin-Based Carriers in Internalization Assays

GE11-conjugated DARPin-based carrier comprising molecules ALB-1C_K27m (SEQ ID NO.: 200, which DARPin-based building block, SEQ ID NO.: 199, comprises a single attachment point, which is the side-chain of a C-terminal Cys), ALB-3C_K27m_w1 (SEQ ID NO.: 173, which DARPin-based building block, SEQ ID NO.: 97, comprises three attachment points, which are the side chains of three Cys) and ALB-5C_K27m (SEQ ID NO.: 174, which DARPin-based building block, SEQ ID NO.: 98, comprises five attachment points, which are the side chains of five Cys) were characterized in an internalization assay via HSA-pHAb specifically binding to Alb23002 (SEQ ID NO.: 106) (molecules ALB-1C_K27m, ALB-3C_K27m_w1 and ALB-5C_K27m comprise an HSA binder which is SEQ ID NO.: 106, see Table 13). For this, NCI-H226 (ATCC CRL-5826) cell line, expressing the epidermal growth factor receptor (EGFR) was used.

To each well of 96-well F-bottom plate (Corning, cat #3596), 100 μL NCI-H226 cells (seeding cell density of 5E+03/96-well) were added in their culture medium: RPMI 1640 (Gibco, cat #72400-021), 10% HI FBS (Sigma-Aldrich, cat #F7524), 1% sodium pyruvate (Gibco, cat #11360-039) and 1% Penicillin-Streptomycin (Gibco, cat #15140-122). Outer wells were not used and were filled with 200 μL D-PBS (Gibco, cat #14190-094).

The 96-well F-bottom plate was placed in the incubator at 37° C., 5% CO₂ for 20-24 hours. Thereafter, serially diluted GE-11-conjugated DARPin compounds (4× concentration) in the cell culture medium were mixed with HSA-pHAb (In House, cat #A8763_PRT1822, 4× concentration—final concentration is 1 μM) in a 1:1 ratio. After 20 minutes at room temperature in the dark, 100 μl serially diluted CARGO-conjugated DARPin construct with HSA-pHAb mix was added to the cells. The 96-well F-plate was placed in the incubator at 37° C., 5% CO₂ for ˜42 hours.

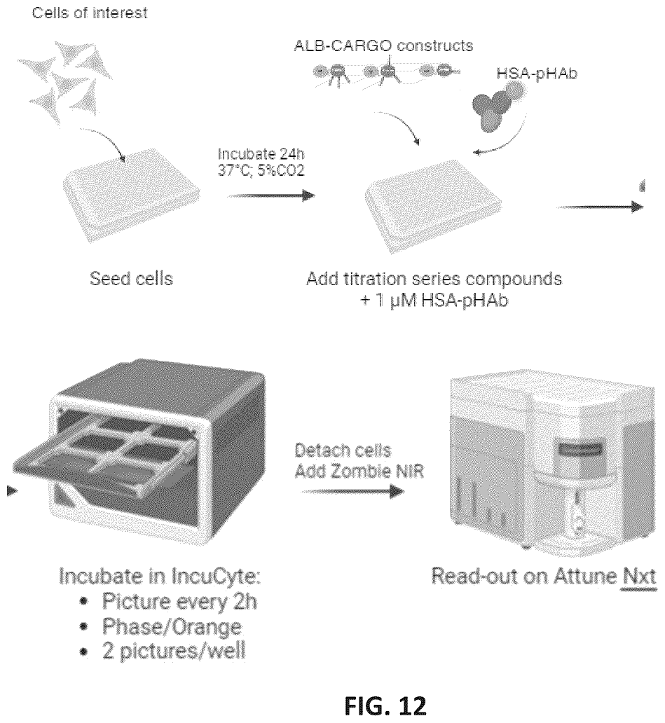

The NCI-H226 cells were harvested after ˜42 hours and transferred to 96-well V-bottom plate (Thermo Scientific, cat #249570). The cells were washed twice with 50 μL/well D-PBS (Gibco, cat #14190-094). Between each washing step, the cells were centrifuged 2 minutes at 300 g at 4° C. Thereafter, the cells were resuspended in 50 μl Zombie NIR™ Fixable Viability Kit (diluted in D-PBS) (BioLegend, cat #423106—final dilution of 1/1000) for 15 minutes at room temperature. After incubation the cells were centrifuged twice for 2 minutes at 300 g at 4° C. and washed with 100 μL/well cold FACS buffer, consisting of D-PBS+2% HI FBS (Sigma-Aldrich, cat #F7524)+0.05% Sodium Azide (Interchim, cat #NJK63A). The cells were diluted in 120 μL FACS buffer and read-out was performed on the Attune N×T (Thermo Fisher Scientific). See FIG. 12, which shows a schematic representation of the cell-internalization assay used.

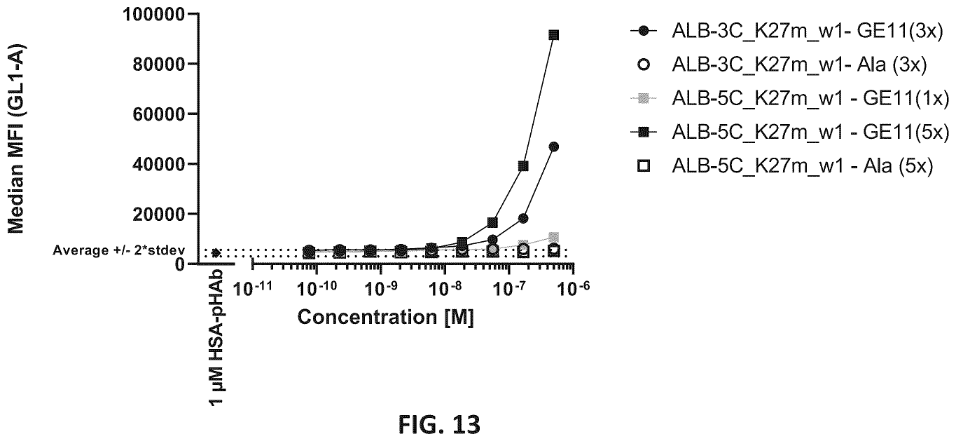

As described above, DARPin-based carrier comprising molecules ALB-3C_K27m_w1 (SEQ ID NO.: 173) and ALB-5C_K27m (SEQ ID NO.: 174) were conjugated with GE11 peptide and internalization was evaluated using NCI-H226 cells, known to express EGFR and was in house confirmed. ALB-3C_K27m_w1 (SEQ ID NO.: 173) was evaluated with DOL (degree of labelling, i.e., number of cargos per carrier) of 3 (vs. 0), while ALB-5C_K27m_w1 (SEQ ID NO.: 174) was evaluated with DOL of 1 and DOL of 5 (vs. 0). As shown in FIG. 13, internalization was noticed with ALB-3C_K27m_w1 (DOL=3) and ALB-5C_K27m_w1 (DOL=5) using NCI-H226 cells at the highest concentrations. Results also illustrate more internalization of ALB-5C_K27m_w1 (DOL=5) compared to ALB-3C_K27m_w1 (DOL=3). No internalization was observed with ALB-5C_K27m_w1 (DOL=1) and all respective negative controls (DOL=0, levels comparable to the baseline set with 1 μM HSA-pHAb).

### Example 12. Folate Conjugation on Molecules Comprising ISVD-Based Carrier Building Blocks

The folate receptor is upregulated in many primary and metastatic cancers and can be used as cell selective penetration strategy for therapeutics by combining folate with drugs that can be transported into cells by means of folate binding and internalization. See, e.g., Marverti, G. et al., “Folic acid-peptide conjugates combine selective cancer cell internalization with thymidylate synthase dimer interface targeting”, Med. Chem. 2021, 64, 6, 3204-3221 and Didion C A and Henne W A, “A Bibliometric analysis of folate receptor research”, BMC Cancer, 2020, 20 (1): 1109 (Erratum in: BMC Cancer. 2021 Jan. 14; 21 (1): 69).

### Preparation of the Constructs

Monomericity was determined by SEC/HPLC. The analysis was performed on a Hitachi Labchrom system equipped with a photodiode array detector and a Superdex 75 increase 5/150 GL column and an isocratic elution of 15 minutes with a pH7 buffer containing 0.2 M of KCl, 0.052 M of KH₂PO₄, 0.107 M of K₂HPO₄ and 20% by volume of isopropanol at a flow rate of 0.2 mL/min.

DAR was determined by reverse phase chromatography—high resolution mass spectrometry (RP/HRMS). The chromatographic analysis was performed on an Acquity UPLC system equipped with a photodiode array detector and an Agilent BioHPLC PLRP-S 4000 Å 5 μm (2.1×50 mm) column at 80° C. with a flow rate of 0.5 mL/min and a gradient elution of (A) H₂O (0.1% HCO₂H) and (B) CH₃CN (0.1% HCO₂H) 95/5 for 7 minutes. The mass spectrometry was performed on a Waters XEVO-G3-QTOF spectrometer with electrospray ionization in positive mode (ES⁺) and mass spectra deconvolution with Waters MaxEnt1 software.

### Preparation of (2S)-2-[[4-[(2-amino-4-oxo-1H-pteridin-6-yl)methylamino]benzoyl]amino]-5-[2-[3-(2,5-dioxopyrrol-1-yl)propanoylamino]ethylamino]-5-oxo-pentanoic acid (folate-aminoethyl-maleimide) (Formula V)

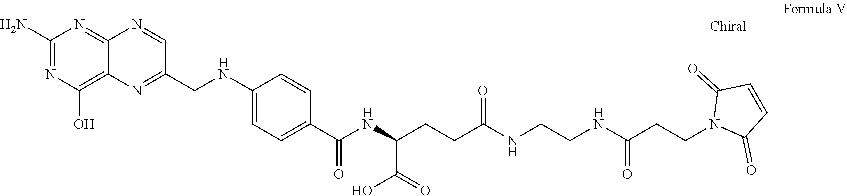 [SMILES: Nc1nc(O)c2nc(CNc3ccc(C(=O)N[C@@H](CCC(=O)NCCNC(=O)CCN4C(=O)C=CC4=O)C(=O)O)cc3)cnc2n1]

### Step 1: Preparation of tert-butyl (2S)-5-[2-(tert-butoxycarbonylamino)ethylamino]-2-(9H-fluoren-9-ylmethoxycarbonylamino)-5-oxo-pentanoate (Intermediate 1)

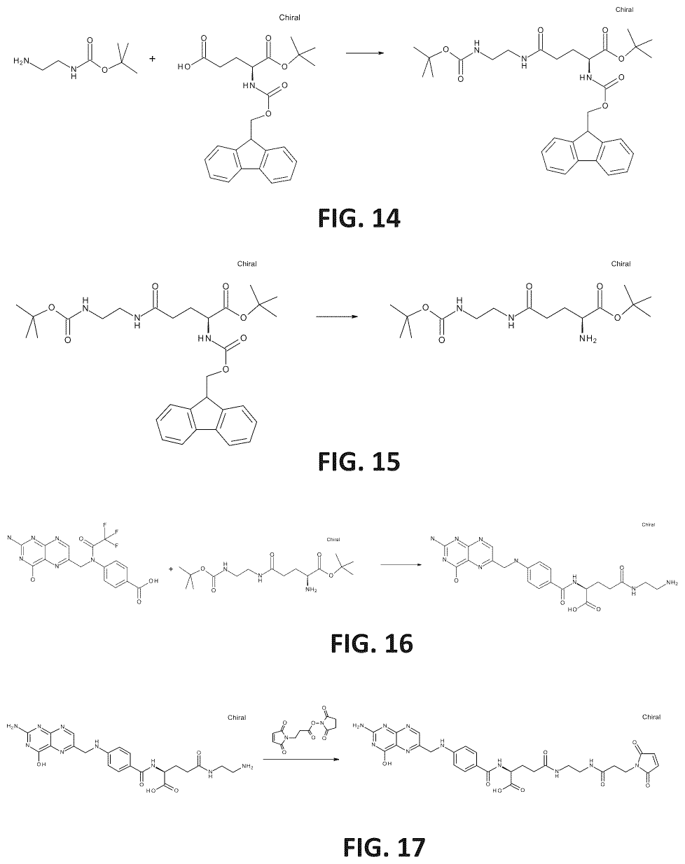

Intermediate 1 was prepared according to a method described in J. Am. Chem. Soc. 2015, 137, 8, 2832. Under a static pressure of argon were successively introduced Fmoc-Glu-OtBu (2 g, 4.7 mmol), DMF (30 mL), HATU (2.68 g, 7.051 mmol), tert-butyl N-(2-aminoethyl) carbamate (753 mg, 4.7 mmol) and N,N-diisopropylethylamine (800 μl, 14.1 mmol). The resulting solution was stirred for 1 hour at room temperature then diluted with ethyl acetate and washed trice with water. The organic phase was dried over MgSO₄, concentrated to dryness then purified by flash chromatography on silica gel (Biotage KP SiL, 340 g), gradient elution of AcOEt/Heptane from 10:90 to 100:0. The compound containing fractions were combined and concentrated to dryness to give the expected product as a white residue (2.12 g, 79%). See FIG. 14.

### Step 2: Preparation of tert-butyl (2S)-2-amino-5-[2-(tert-butoxycarbonylamino)ethylamino]-5-oxo-pentanoate (Intermediate 2)

To a solution of tert-butyl (2S)-5-[2-(tert-butoxycarbonylamino) ethylamino]-2-(9H-fluoren-9-ylmethoxycarbonylamino)-5-oxo-pentanoate obtained from step 1 (1.023 g, 2.96 mmol) in dichloromethane (20 ml) was added piperidine (3.3 ml, 33.6 mmol). The colorless solution was stirred for 1 hour at room temperature then diluted with ethyl acetate and washed trice with water. The organic phase was concentrated to dryness, the residue was triturated in diethylether, filtered over a sintered glass filter and the filtrate concentrated. The residue was then purified by flash chromatography on silica gel (Biotage HP SiL, 120 g), gradient elution of AcOEt/(AcOEt/MeOH, 90:10) from 90:10 to 0:100. The compound containing fractions were combined and concentrated to dryness to give the expected product as a light yellow glassy solid (1.02 g, 88%). See FIG. 15.

### Step 3: Preparation of (2S)-5-(2-aminoethylamino)-2-[[4-[(2-amino-4-hydroxy-pteridin-6-yl)methylamino]benzoyl]amino]-5-oxo-pentanoic acid (Intermediate 3)

To a solution of 4-[(2-amino-4-hydroxy-pteridin-6-yl)methyl-(2,2,2-trifluoroacetyl) amino]benzoic acid (25 mg, 0.061 mmol) in DMF (1.5 mL) were successfully added tert-butyl (2S)-2-amino-5-[2-(tert-butoxycarbonylamino) ethylamino]-5-oxo-pentanoate (25 mg, 0.073 mmol), PYBOP (38 mg, 0.073 mmol) and N,N-diisopropylethylamine (26 μl, 0.135 mmol). The solution was stirred at room temperature for 25 minutes, then diethylether was added up to precipitation of a solid that was collected by filtration over a sintered glass filter. The solid that was collected (47 mg), was further reacted in DMF (1 ml) with a 0.1 M aqueous solution of piperidine (700 μl, 0.061 mmol). After 1 hour at room temperature and 16 hours at +4° C., THF (1 ml) was added followed by diethylether (˜40 ml). The precipitated yellow solid was collected and transferred to a round bottom flask (30 mg) and finally reacted with TFA (0.2 ml) in dichloromethane (0.2 ml). The mixture was stirred for 2.5 hours at room temperature then concentrated to dryness and purified by reverse phase flash chromatography (Macherey Nagel Chromabond™ RS 40 C18 ec) gradient elution of C_{H}3CN/(H2O+0.1% TFA) from 5:95 to 95:5. The compound containing fractions were combined and concentrated to dryness to give the expected product as a light brown solid (11 mg, 37%). See FIG. 16.

### Step 4: Preparation of (2S)-2-[[4-[(2-amino-4-oxo-1H-pteridin-6-yl)methylamino]benzoyl]amino]-5-[2-[3-(2,5-dioxopyrrol-1-yl)propanoylamino]ethylamino]-5-oxo-pentanoic acid (folate-aminoethyl-maleimide)

To a solution of (2S)-5-(2-aminoethylamino)-2-[[4-[(2-amino-4-oxo-1H-pteridin-6-yl)methylamino]benzoyl]amino]-5-oxo-pentanoic acid of step 3 (9 mg, 0.018 mmol) in DMF (1 ml) cooled with an ice bath were added (2,5-dioxopyrrolidin-1-yl) 3-(2,5-dioxopyrrol-1-yl)propanoate (5.3 mg, 0.019 mmol) and N,N-diisopropylethylamine (7.2 μl, 0.041 mmol). The samples was removed from the ice bath and the blurry yellow solution was stirred for 1 hour at room temperature and then purified by reverse phase flash chromatography (Macherey Nagel Chromabond™ RS 25 C18 ec) gradient elution of CH₃CN/(H₂O+0.1% AcOH) from 5:95 to 90:10. The compound containing fractions were combined and freeze dried to give the expected product as a light yellow solid (3 mg, 26%). See FIG. 17. The presence of the folate-aminoethyl-maleimide was confirmed by HPLC/MS ((M1): tR=0.78 mn, ES, m/z=633 [M-H]−).

### Preparation of Folate Conjugates

The molecules comprising ISVD-based carrier building block were expressed and purified as described in Example 4.

To a solution of T028100070 (SEQ ID NO.: 108, comprising a building block based on an ISVD (SEQ ID NO.: 81, which comprises three “wide” Cys, see below) and further comprising an albumin-binding ISVD, SEQ ID NO.: 106, see Table 11) (0.5 ml at 7.58 mg/ml in DPBS, 0.1 mM TCEP) were added 250 μl of DMSO and 129 μl of folate-aminoethyl-maleimide (10 mM in DMSO). The solution was reacted overnight at room temperature, then buffer exchanged via desalting (Sephadex NAP10 column), equilibrated in D-PBS and sterile filtered (Spin-X 0.22 μm) to give the expected conjugate. 1.5 ml at 2.41 mg/ml. The calculated monomeric purity was 100% (DAR(HRMS)=3; RP-HRMS: 28369 (D3)).

To a solution of T028100075 (SEQ ID NO.: 176, comprising a building block based on an ISVD (SEQ ID NO.: 175, which comprises a single C-terminal Cys, see below) and further comprising an albumin-binding ISVD, SEQ ID NO.: 106, see Table 11) (0.25 ml at 12.4 mg/ml in DPBS, 0.1 mM TCEP) were added 35 μl of folate-aminoethyl-maleimide (10 mM in DMSO). The solution was kept 2.5 hours at room temperature, then buffer exchanged via desalting (Sephadex NAP5 column), equilibrated in D-PBS and sterile filtered (Spin-X 0.22 μm) to give the expected conjugate, 0.7 ml at 3.66 mg/ml. The calculated monomeric purity was 100% (DAR(HRMS)=1; RP-HRMS: 27321 (D1)).

In all cases, the folate molecules were linked to the maleimide group via a short diaminoethyl linker resulting in a MW=633 Da/ligand. The results of the conjugation are shown in the below table.

TABLE 14
Analysis of the conjugation of folate to
ISVD-based carrier comprising molecules

| | Concen- | | Concen- | | Total | |
|---|---|---|---|---|---|---|
| | tration | MW | tration | Volume | amount | |
| Molecules | mg/ml | (Dalton) | (μM) | (mL) | (mg) | DOL |
| T028100070 | 2.4 | 28369 | 84.6 | 1.5 | 3.6 | 3 |
| T028100075 | 3.6 | 27321 | 131.7 | 0.7 | 2.5 | 1 |

### Functional Characterization of Molecules Comprising ISVD-Based Carriers in Internalization Assays

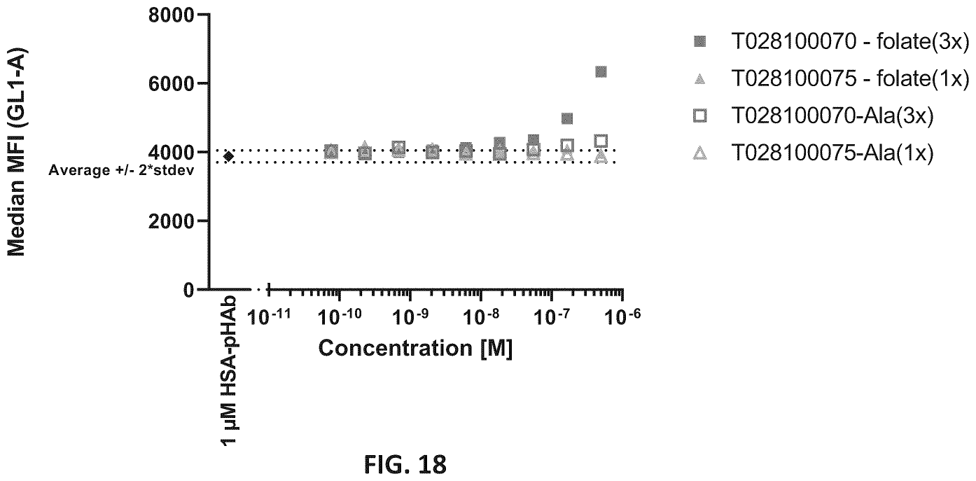

Folate-conjugated ISVD-based carrier comprising molecules T028100070 (SEQ ID NO.: 108) and T028100075 (SEQ ID NO.: 176) were characterized in an internalization assay via HSA-pHAb binding to Alb23002 (SEQ ID NO.: 106), as described above for Example 11. For this, HeLa (ATCC CCL-2) cell line, which expresses the folate receptor (FOLR1) on its surface, as shown in FIG. 18, were used (seeding density: 4E+03/96-well).

To each well of 96-well F-bottom plate (Corning, cat #3596), 100 μl Hela cells (cell density 4E+03/96-well) were added in their culture medium: MEM (Gibco, cat #41090-028), 10% HI FBS (Sigma-Aldrich, cat #F7524), 1% sodium pyruvate (Gibco, cat #11360-039) and 1% Penicillin-Streptomycin (Gibco, cat #15140-122). Outer wells were not used and were filled with 200 μL D-PBS (Gibco, cat #14190-094).

The 96-well F-bottom plate was placed in the incubator at 37° C., 5% CO₂ for 20-24 hours. Thereafter, serially diluted molecules comprising the Folate-conjugated ISVD-derived building block (4× concentration) in the cell culture medium were mixed with HSA-pHAb (In House, cat #A8763_PRT1822, 4× concentration—final concentration is 1 μM) in a 1:1 ratio. After 20 minutes at room temperature in the dark, 100 μl serially diluted CARGO-conjugated ISVD-based comprising molecules, as described above, with HSA-pHAb mix was added to the cells. The 96-well F-plate was placed in the incubator at 37° C., 5% CO₂ for ˜42 hours.

The Hela cells were harvested after ˜42 hours and transferred to 96-well V-bottom plate (Thermo Scientific, cat #249570). The cells were washed twice with 50 μL/well D-PBS (Gibco, cat #14190-094). Between each washing step, the cells were centrifuged 2 minutes at 300 g at 4° C. Thereafter, the cells were resuspended in 50 μl Zombie NIR™ Fixable Viability Kit (diluted in D-PBS) (BioLegend, cat #423106-final dilution of 1/1000) for 15 minutes at room temperature. After incubation the cells were centrifuged twice for 2 minutes at 300 g at 4° C. and washed with 100 μL/well cold FACS buffer, consisting of D-PBS+2% HI FBS (Sigma-Aldrich, cat #F7524)+0.05% Sodium Azide (Interchim, cat #NJK63A). The cells were diluted in 120 μL FACS buffer and read-out was performed on the Attune N×T (Thermo Fisher Scientific).

Internalization of T028100070-Folate (3×) and T028100075-Folate (1×) was evaluated on HeLa cells, Folate receptor 1 (FOLR1) expression on Hela cells was in house confirmed. At the highest concentration of T028100070-Folate (3×), limited internalization was observed using Hela cells compared to its respective negative control, T028100070-Ala (3×) (levels comparable to the baseline set with 1 μM HSA-pHAb) (FIG. 18). No internalization was noticed with T028100075-Folate (1×) and its corresponding negative control T028100075-Ala (1×).

### Example 13. Mannose 6 Phosphate (M6P) Conjugation on Molecules Comprising ISVD-Based Carrier Building Blocks

### Preparation of the Constructs

As discussed above, targeted protein degradation strategies are gaining importance for new therapeutic strategies. Via the conjugation of multiple bisM6P molecules to the protein-based carrier building blocks, proteins are directed to the lysosomes in target cells for lysosomal degradation.

Monomericity was determined by SEC/HPLC. The analysis was performed on a Hitachi Labchrom system equipped with a photodiode array detector and a Superdex 75 increase 5/150GL column and an isocratic elution of 15 minutes with a pH7 buffer containing 0.2 M of KCl, 0.052 M of KH2PO4, 0.107 M of K2HPO4 and 20% by volume of isopropanol at a flow rate of 0.2 mL/min.

Degree Of Labelling (DOL) was determined by reverse phase chromatography—high resolution mass spectrometry (RP/HRMS). The chromatographic analysis was performed on an Acquity UPLC system equipped with a photodiode array detector and an Agilent BioHPLC PLRP-S 4000 Å, 5 μm (2.1×50 mm) column at 80° C. with a flow rate of 0.5 mL/min and a gradient elution of (A) H2O (0.1% HCO2H) and (B) C_{H}3CN (0.1% HCO2H) 95/5 for 7 minutes. The mass spectrometry was performed on a Waters XEVO-G3-QTOF spectrometer with electrospray ionization in positive mode (ES+) and mass spectra deconvolution with Waters MaxEnt1 software.

Preparation of bisM6P-Mal Drug-Linker

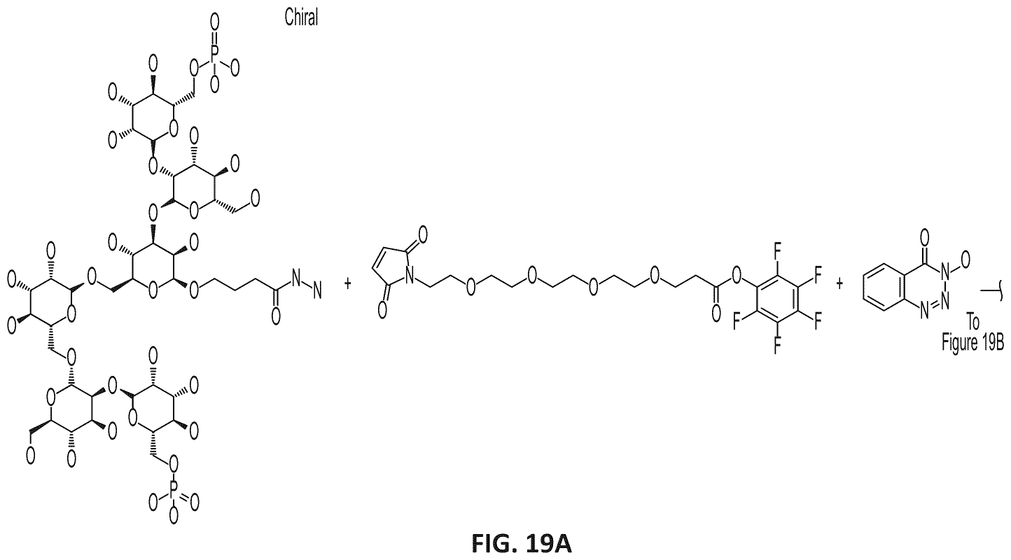

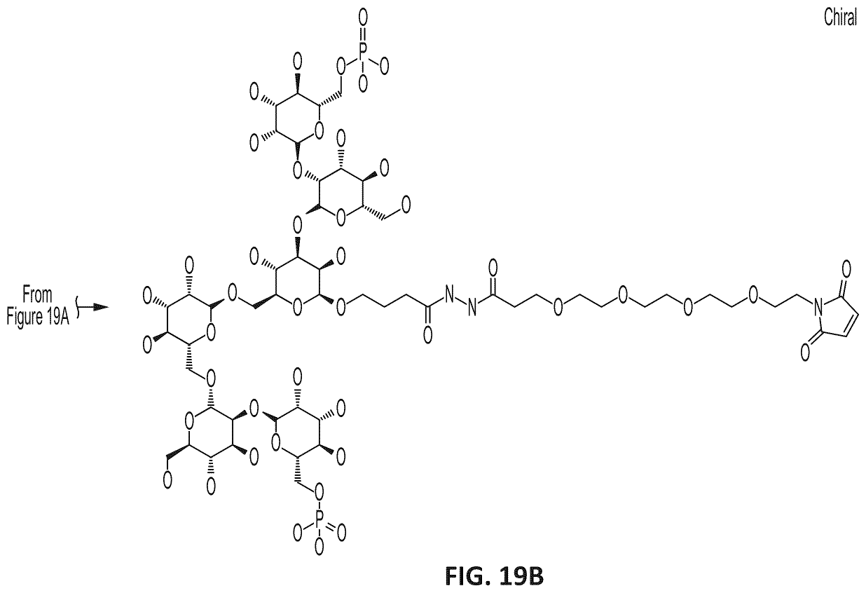

A schema of the synthesis of the bisM6P ([(2S,3R,4R,5R)-6-[(3S,4S,5S,6R)-2-[[(2R,3S,4S,5S)-6-[[(2S,3S,4R,5R,6S)-4-[(3R,4R,5R,6S)-4,5-dihydroxy-6-(hydroxymethyl)-3-[(3R,4R,5R,6S)-3,4,5-trihydroxy-6-(phosphonooxymethyl)tetrahydropyran-2-yl]oxy-tetrahydropyran-2-yl]oxy-6-[4-[2-[3-[2-[2-[2-[2-(2,5-dioxopyrrol-1-yl)ethoxy]ethoxy]ethoxy]ethoxy]propanoyl]hydrazino]-4-oxo-butoxy]-3,5-dihydroxy-tetrahydropyran-2-yl]methoxy]-3,4,5-trihydroxy-tetrahydropyran-2-yl]methoxy]-4,5-dihydroxy-6-(hydroxymethyl)tetrahydropyran-3-yl]oxy-3,4,5-trihydroxy-tetrahydropyran-2-yl]methyl dihydrogen phosphate) is shown in FIGS. 19A and 19B.
bisM6P-PEG4-mal drug linker was prepared by analogy to a method described in WO 2010/075010 A2. To a solution of MAL-PEG4-PFP (41 mg, 0.0802 mmol) in DMSO (0.5 ml) were added DHBT (13 mg, 0.0797 mmol) and a solution of bisM6P (20 mg, 0.016 mmol) in water (0.5 ml). The light yellow solution was stirred for 1 hour at room temperature then purified by size exclusion chromatography (LH20 resin, 70 g) equilibrated with water. The product-containing fractions were combined and freeze-dried to give the expected product as a white lyophilyzate (19 mg, 76%).

The presence of the bisM6P-PEG4-mal drug linker was confirmed by HPLC/MS ((M1): tR=4.58 mn, ES, m/z=788 [M+2H]2+).

Mal-PEG4-PFP is a PEG linker with maleimide and pentafluorophenyl (PFP) moieties, which structure is depicted in Formula (IV) below:

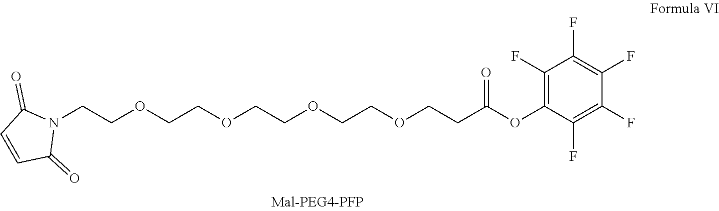 [SMILES: O=C(CCOCCOCCOCCOCCN1C(=O)C=CC1=O)Oc1c(F)c(F)c(F)c(F)c1F]

Preparation of bisM6P-PEG4-Conjugates on ISVD-Comprising Molecules

The molecules comprising ISVD-based carrier building block were expressed and purified as described in Example 4.

To a solution of T028100069 (SEQ ID NO.: 107, comprising a building block based on an ISVD, SEQ ID NO.: 80, and further comprising an albumin-binding ISVD) (2 ml at 2.65 mg/ml in DPBS, 2 mM EDTA) were added 161 μl of bisM6P-PEG4-mal (10 mM in DPBS). The solution was kept overnight at room temperature then purified via size exclusion chromatography (HiLoad 16/60 Superdex™ 75 pg) equilibrated in D-PBS. The product containing fractions were pooled, spin concentrated (Amicon Ultra-15, Ultracel 10 kD) and sterile filtered (Millex 0.22 μm) to give the expected conjugate, 1.5 ml at 3.36 mg/ml. The calculated monomeric purity was 100% (DAR(HRMS)=2.9; RP-HRMS: 29218 (D2); 31114 (D3)).

Multiple maleimide-activated bisM6P molecules (see Formula VII) were conjugated onto the free thiols of the building blocks.

The building blocks were expressed and purified as described in Example 4. The engineered cysteines present in the ISVD-based building block (SEQ ID NO.: 80)-comprising molecule (SEQ ID NO.: 107), in the ISVD-based building block (SEQ ID NO.: 81)-comprising molecule (SEQ ID NO.: 108) or in the control molecule (SEQ ID NO.: 176, which protein-based building block (SEQ ID NO.: 175) comprises a C-terminal Cys with a —SH in its side chain which is the attachment point of this building block) were reduced and/or uncapped using 10 mM in DTT PBS, after which DTT was separated from the molecule solution (comprising the ISVD-building block with reduced engineered cysteines) via HiPrep™ 26/10 Desalting (Cytiva 17-5087-01) or Size Exclusion Chromatography (SEC) on Superdex® Increase 75 10/300 GL (Cytiva 29-1487-21), equilibrated in D-PBS with 0.1 mM TCEP (20490 Thermo Scientific™ Pierce™). This material can be used directly for conjugations or frozen at −20° C. to be used later (TCEP will prevent reoxidation of the free thiols).

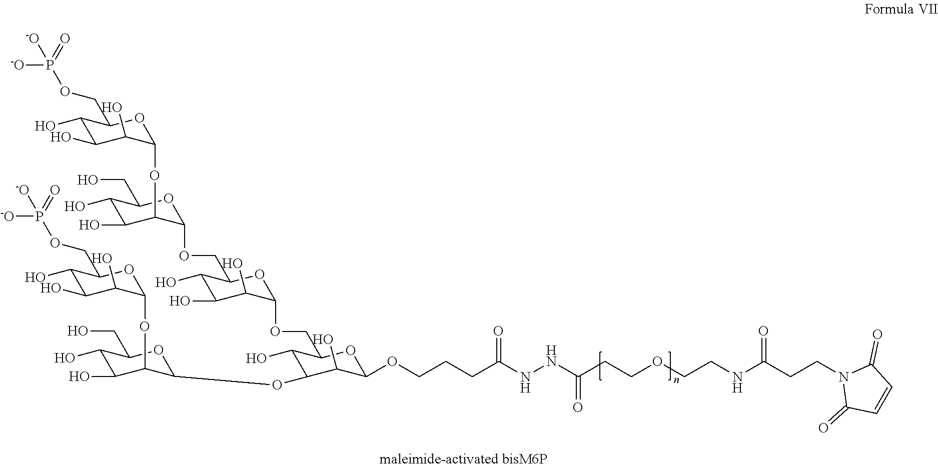

For maleimide-based conjugation of bisM6P, the same protocol as described in Example 5.1 was used. Carrier molecules (SEQ ID NOs.: 107, 108 and 176) were uploaded with maleimide-bisM6P using generic conditions with excess ligand, as explained in detail above in these examples, generating DOLs of 1 (SEQ ID NO.: 176) and 3 (SEQ ID Nos.: 107 and 108). The bisM6P molecules were linked to the maleimide group via a short PEG linker (n=4), resulting in a MW=1577 Da, see Table 15.

To a solution of T028100070 (SEQ ID NO.: 108, comprising a building block based on an ISVD, SEQ ID NO.: 81, and further comprising an albumin-binding ISVD, SEQ ID NO.: 106) (1 ml at 7.58 mg/ml in DPBS, 0.1 mM TCEP) were added a total of 258 μl of bisM6P-PEG4-mal (10 mM in DPBS). The solution was kept overnight at room temperature then purified via size exclusion chromatography (HiLoad 16/60 Superdex™ 75 pg) equilibrated in D-PBS. The product containing fractions were pooled, spin concentrated (Amicon Ultra-15, Ultracel 10 kD) and sterile filtered (Millex 0.22 μm) to give the expected conjugate. 1.2 ml at 4.04 mg/ml. The calculated monomeric purity was 100% (DAR(HRMS)=2.84; RP-HRMS: 29674 (D2); 31200 (D3)).

To a solution of T028100069 (SEQ ID NO.: 107, comprising a building block based on an ISVD, SEQ ID NO.: 80, and further comprising an albumin-binding ISVD, SEQ ID NO.: 106) (1 ml at 7.58 mg/ml in DPBS, 0.1 mM TCEP) were added a total of 258 μl of bisM6P-PEG4-mal (10 mM in DPBS). The solution was kept overnight at room temperature then purified via size exclusion chromatography (HiLoad 16/60 Superdex™ 75 pg) equilibrated in D-PBS. The product containing fractions were pooled, spin concentrated (Amicon Ultra-15, Ultracel 10 kD) and sterile filtered (Millex 0.22 μm) to give the expected conjugate. 1.5 ml at 3.36 mg/ml. The calculated monomeric purity was 100% (DAR(HRMS)=2.9; RP-HRMS: 29674 (D2); 31200 (D3)).

To a solution of T028100075 (SEQ ID NO.: 176, comprising a building block based on an ISVD, SEQ ID NO.: 175, comprising a C-terminal Cys with a —SH in its side chain which is the attachment point of this building block, and further comprising an albumin-binding ISVD, SEQ ID NO.: 106) (0.5 ml at 12.4 mg/ml in DPBS, 0.1 mM TCEP) were added 0.5 ml of DPBS and a total of 58 μl of bisM6P-PEG4-mal (10 mM in DPBS). The solution was kept overnight at room temperature then purified via size exclusion chromatography (HiLoad 16/60 Superdex™ 75 pg) equilibrated in D-PBS. The product containing fractions were pooled, concentrated to a volume of 1.5 ml using Amicon Ultra-15 (Ultracel 10 kD) and sterile filtered (Millex 0.22 μm) to give the expected conjugate, 1.4 ml at 3.3 mg/ml. The calculated monomeric purity was 100% (DAR(HRMS)=1.00; RP-HRMS: 28265 (D1)).

TABLE 15
Analysis of the conjugation of bisM6P to
ISVD-based carrier comprising molecules

| | | | | | | DOL |
|---|---|---|---|---|---|---|
| Carrier | Conc | Conc | V | Total | Monomeric purity | Mass |
| molecules | mg/ml | HM | (mL) | mg | SEC HPLC | Spec |
| T028100069 | 3.36 | 127 | 1.5 | 5.04 | 100 | 2.90 |
| T028100070 | 4.04 | 129 | 1.2 | 4.85 | 100 | 2.84 |
| T028100075 | 3.30 | 117 | 1.4 | 4.62 | 100 | 1.00 |

As negative control, the above building blocks were conjugated to maleimide-Ala instead of maleimide-bisM6P. In addition, an ISVD specifically binding M6PR was used as positive control, and an ISVD which does not specifically bind M6PR was used as a further negative control.

### Cell Binding Assay

The bisM6P-conjugated molecules as described above were tested for cell binding and cell internalization via pHAb labeled albumin as test item. In this case, the cell line used was K-562 (ATCC CCL-243). Expression of mannose 6-phosphate receptor (M6PR) on K-562 cells was in house confirmed and therefore, these cells were selected to evaluate the binding of ISVD-Carrier compounds with bis-M6P.

To each well of a 96-well V-bottom plate (Thermo Scientific, cat #249570), 50 μL cell suspension (5E+04 cells/96-well) in cold FACS buffer, consisting of D-PBS (Gibco, cat #14190-094)+2% Heat Inactivated Fetal Bovine Serum (HI FBS) (Sigma-Aldrich, cat #F7524)+0.05% Sodium Azide (Interchim, cat #NJK63A), was added. After a washing step, 50 μL serially diluted bisM6P-conjugated molecules as described above were added to the cells and incubated for 3 hours at 4° C., shaking.

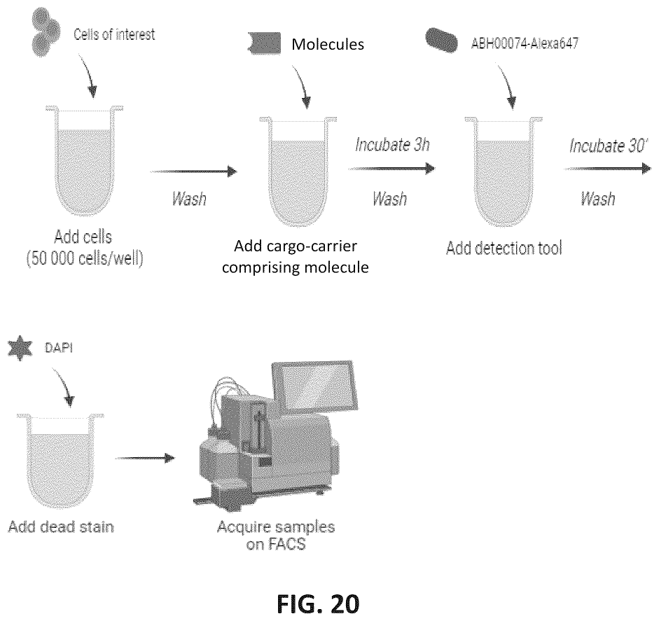

The binding of bisM6P-conjugated molecules comprising ISVD-derived carriers T028100069 (SEQ ID NO.: 107), T028100070 (SEQ ID NO.: 108) and T028100075 (SEQ ID NO.: 176) (bis-M6P) vs ISVD-Ala (Alanine conjugated) was evaluated via HSA binding to Alb23002 (SEQ ID NO.: 106). The cells were stained in 50 μL Albumin from human serum-Biotin (In House, cat #A8763_PRT2572—final concentration of 1 μM) for 30 minutes at 4° C. Thereafter, the cells were stained with Streptavidin-Phycoerythrin (BD-Pharmingen, cat #554061-final dilution of 1/1000). Between each step, the cells were centrifuged twice for 2 minutes at 300 g at 4° C. and washed with 100 μL/well cold FACS buffer. The cells were diluted in 50 μL DAPI solution (BD Biosciences, cat #564907-final dilution of 1/5000) in cold FACS buffer and read-out was performed on MACSQuant X (Miltenyi Biotec). See FIG. 20.

As negative control, the same molecules T028100069 (SEQ ID NO.: 107), T028100070 (SEQ ID NO.: 108) and T028100075 (SEQ ID NO.: 176) but conjugated with maleimide-alanine instead of bisM6P were used. The positive control was an ISVD specific for M6PR. A further negative control (ISVD not specifically binding M6PR was also used.

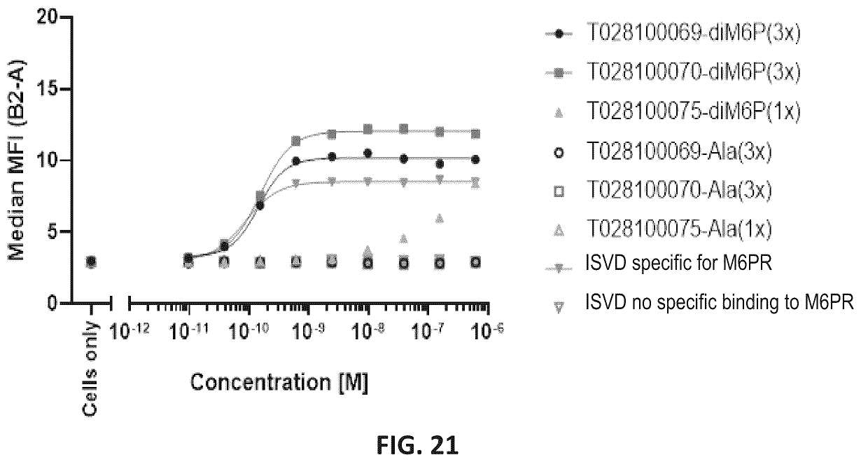

The K-562 binding results are shown in FIG. 21. As illustrated in this figure, complete dose-response curves were obtained with T028100069-bis-M6P (3×) and T028100070-bis-M6P (3×) with a similar binding affinity of ˜1.50E-10 M, as shown in Table 16. These results indicate that clustered vs wide bis-M6P conjugation do not influence the binding towards M6PR, likely via the added flexibility of the PEG linker. Only weak binding of T028100075-bis-M6P (1×) to K-562 cells was observed. As expected, all ISVD-derived carrier compounds conjugated with Alanine did not show any binding to K-562 cells, illustrating the specific binding of ISVD-derived carrier compounds with bis-M6P to the cells. These cell binding results indicate an increase of more than 2 orders of magnitude in specific cell binding for molecules comprising 3 attachment points occupied by bisM6P (DOL 3) than for molecules with a single attachment point occupied by bisM6P (DOL 1)

TABLE 16
Overview of binding affinities of ISVD-Carrier
compounds with bis-M6P on K-562 cells.

| ISVD-Carrier compound | EC50 [M] |
|---|---|
| T028100069-bis-M6P(3x) | 1.43E−10 |
| T028100070-bis-M6P(3x) | 1.54E−10 |
| T028100075-bis-M6P(1x) | iDRC |

iDRC: incomplete dose-response curve

### Functional Characterization of Molecules Comprising ISVD-Based Carriers in Internalization Assays

bisM6P-conjugated ISVD-based carrier comprising molecules T028100069 (SEQ ID NO.: 107), T028100070 (SEQ ID NO.: 108) and T028100075 (SEQ ID NO.: 176) were characterized in an internalization assay via HSA-pHAb binding to Alb23002 (SEQ ID NO.: 106) present in the protein-based carrier molecule, as described above for Example 11. For this, K-562 (ATCC CCL-243) cell line was used.

10 μl of the detection tool, HSA-pHAb (In House, cat #A8763_PRT1822), and 10 μl of serially diluted of the above-described molecules were added to a 384-well Flat Clear Bottom plate (Corning, cat #3764). The plate was shortly centrifuged to ensure that all media was at the bottom of the well. To each well, 20 μl of cells (5E+03/384-well) in assay medium, consisting of RPMI 1640 (Gibco, cat #72400), 10% FBS HI (Sigma-Aldrich, cat #F7524), 1× sodium pyruvate (Gibco, cat #11360-039) and 1% Penicillin-Streptomycin (P/S) (Gibco, cat #15070-063), were added. Outer wells were not used and were filled with 40 μL assay medium. After 15 minutes at room temperature, the plate was placed in the incubator at 37° C., 5% CO₂ for 42 hours.

The cells were harvested after 42 hours and transferred to a 348-well V-bottom plate (Greiner, cat #781280). Thereafter, the cells were resuspended in 50 μL Zombie NIR™ Fixable Viability Kit (diluted in PBS) (BioLegend, cat #423106) for 15 minutes at room temperature. Between each step, the cells were centrifuged 3 times for 2 minutes at 300 g at 4° C. and washed with 50 μL/well FACS buffer, consisting of D-PBS (Gibco, Cat #14190-094)+2% HI FBS (Sigma-Aldrich, Cat #F7524)+0.05% Sodium Azide (Acros Organics, cat #NJK63A), using the VIAFLO 384 (INTEGRA Biosciences) and BioTek Washer (Agilent). The cells were diluted in 80 μL FACS buffer and read-out was performed on the Attune N×T (Thermo Fischer Scientific).

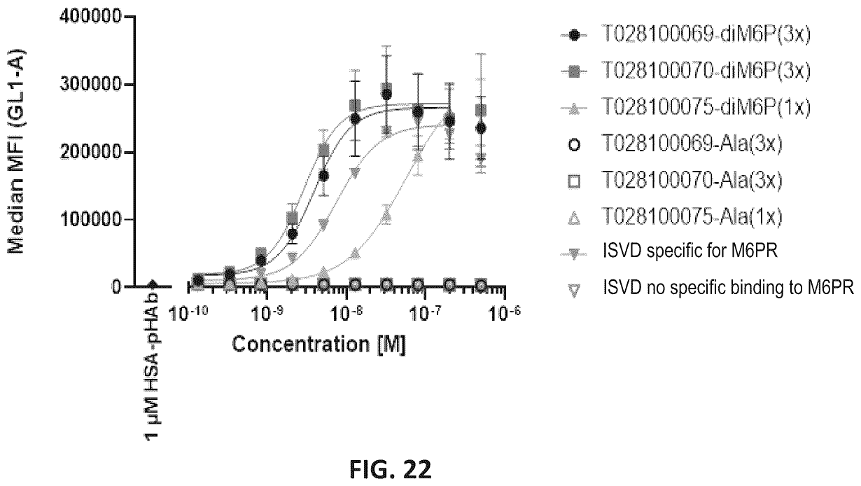

The results are shown in FIG. 22. As illustrated in this figure, internalization was observed for all bis-M6P conjugated ISVD-cased building block comprising molecules. The best internalization potency (2-3 nM) was obtained with T028100069-bis-M6P(3×) and T028100070-bis-M6P(3×), and no differences were noticed between wide vs clustered bis-M6P conjugation, as illustrated in Table 17. A ˜20-fold decrease in internalization potency was obtained with T028100075-bis-M6P(1×). Their respective negative controls did not internalize and are comparable to the baseline set with 1 μM HSA-pHAb. The internalization data confirm previous observations in flow cytometry (see FIG. 21).

TABLE 17
Overview of internalization potencies of ISVD-
Carrier compounds with bis-M6P on K-562 cells.

| ISVD-Carrier compound | EC50 [M] |
|---|---|
| T028100069-bis-M6P(3x) | 3.86E−09 |
| T028100070-bis-M6P(3x) | 2.88E−09 |
| T028100075-bis-M6P(1x) | 5.64E−08* |

*Indicative value

Hence, multiple bisM6P molecules conjugated to the protein-based carrier building block comprised in the molecule of the present technology are able to specifically bind the M6PR and efficiently internalize in cells expressing the same. Hence, these molecules could be used, e.g., in combination with a targeting biological (e.g., via the presence of a further cargo in the molecule, e.g., by genetic fusion construct) to internalize targets to be destroyed via the lysosomal pathway, following a similar approach as LYTACs, lysosome-targeting chimeras.

### Example 14. Lipid Conjugation on Molecules Comprising ISVD- and CKS-Based Carrier Building Blocks

### Preparation of the Constructs

The following maleimide-activated lipids were used:

### Short-Chain Lipids

a) 6-Maleimidohexanoic acid (Thermo Scientific Chemicals, product code 15482677); and
b) 11-Maleimidoundecanoic acid (Thermo Scientific Chemicals, product code 15492747).

The lipids were dissolved MeOH and conjugated onto two different building blocks as described below:

ISVD-derived building block with SEQ ID NO.: 86, comprising 6 Cys available for conjugation (solvent accessible), see Table 5, comprised in molecule T028100078 (SEQ ID NO.: 113). The short-chain lipids were conjugated on the ISVD-derived building block with SEQ ID NO.: 86, comprised in molecule T028100078 (SEQ ID NO.: 113), which further comprises an albumin-binding ISVD with SEQ ID NO.: 106, see Table 11). The ISVD-derived building block with SEQ ID NO.: 83 comprises six attachment points or conjugation sites which are the —SH groups of six cysteines. The following constructs were generated (i) DOL0 (T028100078 with no lipid), DOL3 (only 3 out of the 6 Cys were occupied by lipids, in a stochastic manner) and DOL6 (all 6 Cys were occupied by lipids); and
CKS-derived building block with SEQ ID NO.: 101, comprising 3 Cys, see Table 7, comprised in molecule ALB-3C_hCKS1_c3 (SEQ ID NO.: 125), using generic conditions (e.g., as described in Example 5.1). Then, the conjugated constructs were purified via SEC (Superdex 75 Increase column equilibrated in D-PBS,), eluted in D-PBS and characterized via SDS-PAGE and Mass Spec. Mass Spec analysis did confirm the conjugation of the short-chain lipids (hexanoic acid and undecanoic acid) on the ISVD- and CKS-derived building block comprised in T028100078 (SEQ ID NO.: 113) and ALB-3C_hCKS1_c3 (SEQ ID NO.: 125), confirming the successful conjugation of resp. 6 and 3 lipid molecules per building block.

The solution comprising the molecules as described above (at 1.64 mg/ml, 2 mg total) was thawed from −80° C. and the maleimide activated lipids (dissolved in MeOH) were added at a 7.2 molar ratio. Two separate DOL batches were conjugated. The negative control was conjugated with maleimide-Alanine at a 15× molar ratio; conjugation mixes were incubated for 2 hours at room temperature. The reaction mix were separated using SEC (Superdex 200) equilibrated in D-PBS. The peak fractions were collected and stored at 4° C. for further analysis. SDS-PAGE was carried out on the SEC fractions (not shown) and selected peak fractions were pooled, quantified and analyzed via Mass Spec. Stocks were made and frozen at −60° C.

SEC Analysis was performed on an AKTAmicro (Cytiva). The column used was Superdex 75. The increase was 5/150, and the running buffer was D-PBS, with a flow rate of 0.3 ml/min. The sample injection loop was 20 μL. Sample mixtures of 10 μg HSA & 3 μg V_{HH} were injected in the column to run the chromatography.

### LC-MS Method

LC system: Ultimate 3000 HPLC system (Thermo Fisher Scientific) equipped with a Zorbax Poroshell 300SB-C8 column (5 μm, 300 Å, 1×75 mm ID×L; Agilent Technologies). Column oven temperature was set at 60° C. Chromatography conditions: flow rate: 100 μl/min; solvent A: 0.1% formic acid and 0.05% trifluoroacetic acid in water and solvent B: 0.1% formic acid and 0.05% trifluoroacetic acid in acetonitrile. A gradient of 5% to 80% solvent B in 5 min was applied.

MS system: LTQ Orbitrap Elite mass spectrometer (Thermo Fisher Scientific), analyzer: Orbitrap, resolution: 60,000 (at m/z 400), mass range: 600-4000 m/z (profile mode). The settings on the mass spec were ats follows: ESI source: spray voltage: 4.2 kV, surface induced dissociation: 30 V, capillary temperature: 325° C., capillary voltage: 35 V and Sheath gas flow rate: 7 (arbitrary units). Data analysis software: BioPharma Finder™ 3.0 software (Thermo Fisher Scientific) and deconvolution algorithm: Xtract/ReSpect.

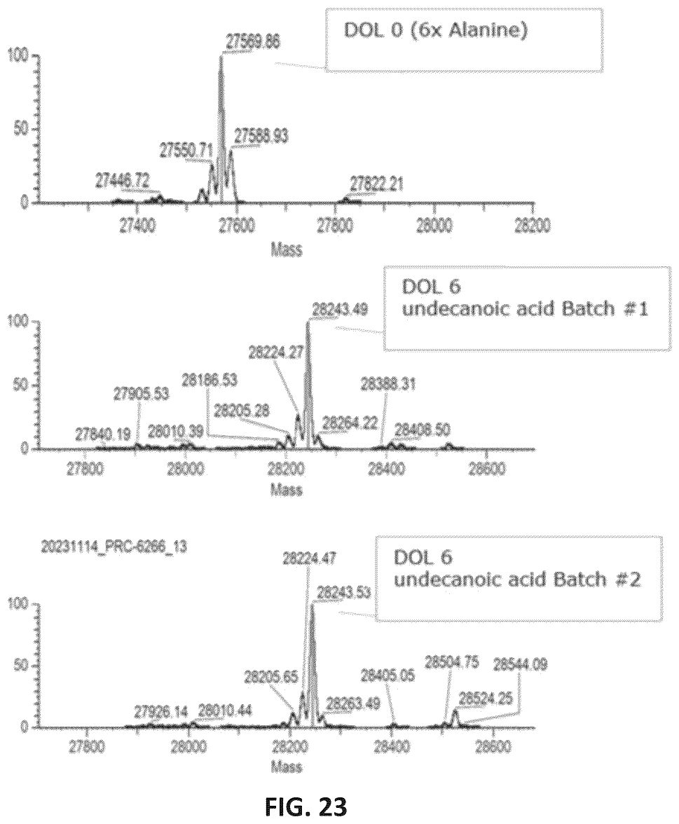

The generation of the above-indicated molecules was confirmed by mass spectrometry (data not shown and FIG. 23).

### Functional Characterization of Molecules Comprising ISVD- and CKS-Based Carriers in Internalization Assays

To each well of 96-well F-bottom plate (Corning, cat #3596), 100 μL BxPC-3 cells (seeding cell density of 1.2E+04/96-well) were added in their culture medium: RPMI 1640 (Gibco, cat #72400-021), 10% HI FBS (Sigma-Aldrich, cat #F7524), 1% sodium pyruvate (Gibco, cat #11360-039) and 1% Penicillin-Streptomycin (Gibco, cat #15140-122). Outer wells were not used and were filled with 200 L D-PBS (Gibco, cat #14190-094).

The 96-well F-bottom plate was placed in the incubator at 37° C., 5% CO₂ for 20-24 hours. Thereafter, serially diluted molecules comprising lipid-conjugated ISVD- and CKS-derived building blocks (4× concentration) in the cell culture medium were mixed with HSA-pHAb (In House, cat #A8763_PRT1822, 4× concentration—final concentration is 1 μM) in a 1:1 ratio. After 20 minutes at room temperature in the dark, 100 μl serially diluted molecules comprising lipid-conjugated ISVD- and CKS-based building blocks with HSA-pHAb mix was added to the cells. The 96-well F-plate was placed in the incubator at 37° C., 5% CO₂ for ˜42 hours.

The BxPC-3 cells were harvested after ˜42 hours and transferred to 96-well V-bottom plate (Thermo Scientific, cat #249570). The cells were washed twice with 50 μL/well D-PBS (Gibco, cat #14190-094). Between each washing step, the cells were centrifuged 2 minutes at 300 g at 4° C. Thereafter, the cells were resuspended in 50 μL Zombie NIR™ Fixable Viability Kit (diluted in D-PBS) (BioLegend, cat #423106—final dilution of 1/1000) for 15 minutes at room temperature. After incubation the cells were centrifuged twice for 2 minutes at 300 g at 4° C. and washed with 100 μL/well cold FACS buffer, consisting of D-PBS+2% HI FBS (Sigma-Aldrich, cat #F7524)+0.05% Sodium Azide (Interchim, cat #NJK63A). The cells were diluted in 120 μL FACS buffer and read-out was performed on the Attune N×T (Thermo Fisher Scientific).

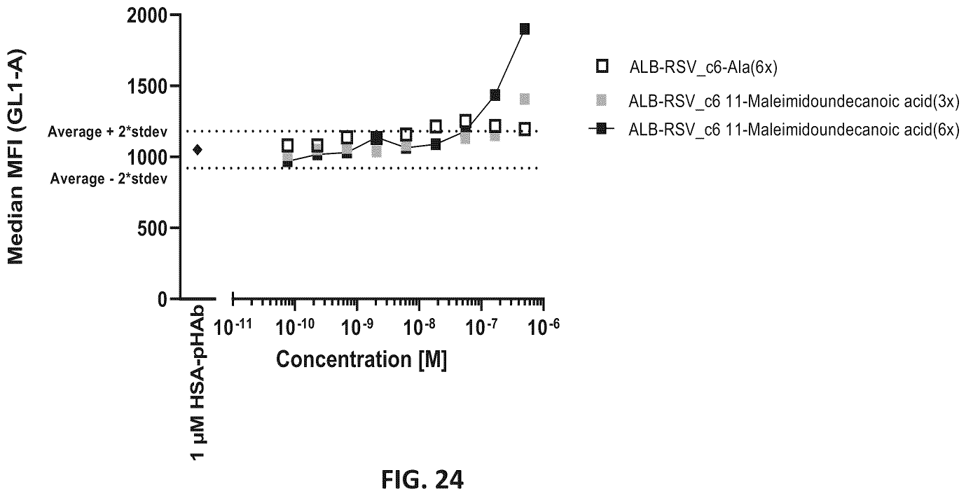

Internalization of T028100078 (SEQ ID NO.: 113, also referred to as “ALB-RSV_c6”) conjugated with 11-Maleimidoundecanoic acid was evaluated using BxPC-3 cells. Different DOLs were tested of ALB-RSV_c6: DOL of 3 and DOL of 6 (vs 0, i.e., Ala-conjugated molecules). See FIG. 24. At the highest concentration of ALB-RSV_c6 conjugated with 11-Maleimidoundecanoic acid (DOL=6), internalization was observed using BxPC-3 cells compared to its respective negative control, ALB-RSV_c6 (DOL=0), as levels were comparable to the baseline set with 1 μM HSA-pHAb.

The results suggest a lipid membrane interaction which favors a target independent cell uptake of the carrier with higher DOLs of undecanoic acid which is detected in endosomal condition via pHAb labeled albumin.

### Example 15. PEG Conjugation on Molecules Comprising ISVD-Based Carrier Building Blocks

PEGylation is used to increase the serum half-life (HLE) of drugs or therapeutic proteins or peptides, decrease frequency of drug administration, improve efficacy, and reduce the immunogenicity of the proteins or peptides, see, e.g., Fang JL. et al., “Toxicity of high-molecular-weight polyethylene glycols in Sprague Dawley rats”, Toxicol Lett., 2022, 359:22-30.

PEGs with molecular weights of up to 7.5 kDa demonstrate a low level of toxicity in acute, short-term, and long-term studies using oral, intraperitoneal, and intravenous routes in a wide range of animal species. High-molecular-weight PEGs show slow renal clearance and consequently have a greater potential to accumulate within cells. There is significant concern that the accumulation of high-molecular-weight PEGs and cellular vacuolization may lead to adverse outcomes for PEGylated biologicals used at high dose, chronically and/or in pediatric populations. Via the conjugation of several small MW PEG molecules (i.e., 5 kDa) to a carrier molecule (molecule comprising a protein-based building block), sufficient size to bypass renal clearance is obtained, giving HLE and, more importantly, avoiding accumulation typically associated with larger PEG moieties.

### Preparation of the Constructs

Maleimide-5 kDa PEG was conjugated on the ISVD-derived building block with SEQ ID NO.: 90, which comprises 6 cysteines which side chain are the 6 attachment points of this building block (see Table 5), comprised in molecule T028100118 (SEQ ID NO.: 117), see Table 11.

Maleimide-5 kDa PEG and Maleimide-30 kDa PEG were conjugated on the ISVD-derived building block with SEQ ID NO.: 175, with the same maleimide chemistry as described in Example 5.3. The ISVD-derived building block with SEQ ID NO.: 175 comprises a single C-terminal cysteine, which side chain is the single attachment point of this building block (see Table 5), comprised in molecule T028100075 (SEQ ID NO.: 176), see Table 11.

For all conjugations, methoxy-capped PEG was used; both 5 kDa PEG and 30 kDa PEG were acquired from BroadPharm, resp. https://broadpharm.com/product/bp-23356 and https://broadpharm.com/product/bp-23356 Conjugations were carried out using generic conjugation conditions using an excess of maleimide-activate PEG, followed by removal of excess unconjugated PEG via cation exchange and SEC and formulated in D-PBS.

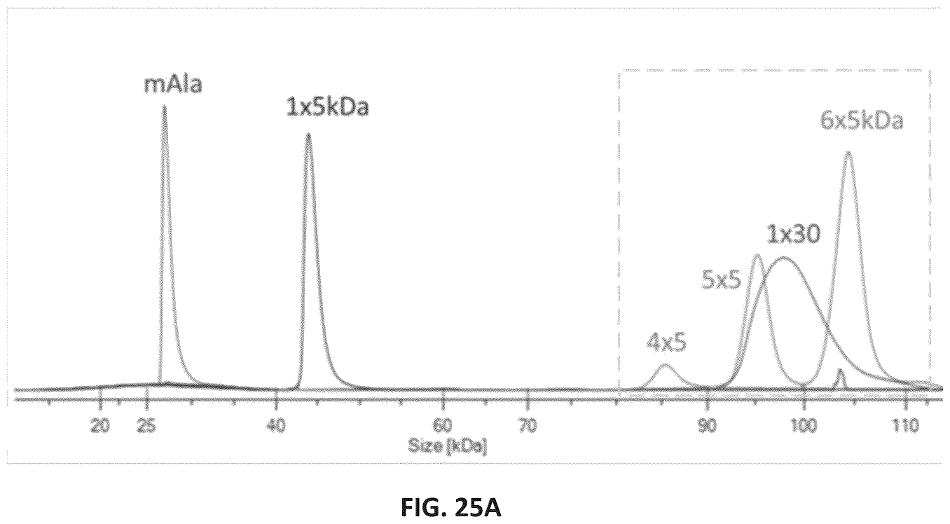

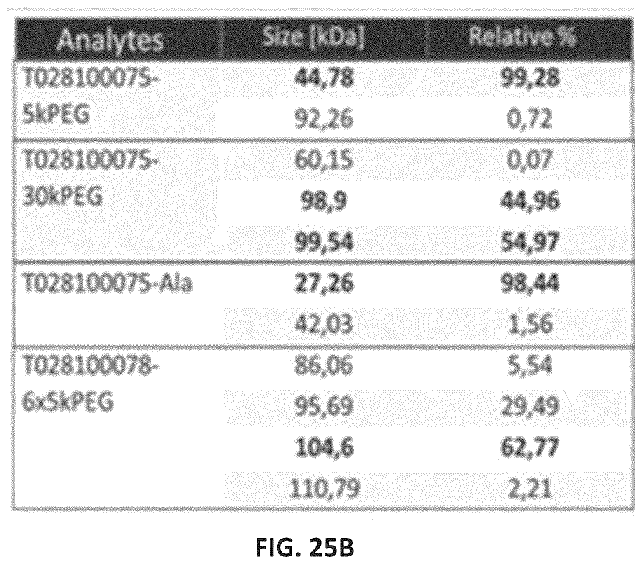

The obtained molecules were characterized using the LabChip® GXII Touch™ HT Protein Characterization System (https://www.perkinelmer.com/product/ship-level-labchip-gx-ii-touch-ht-cls138160), which provides a complete solution for reproducible quantitation, molecular weight sizing and percent purity analysis of protein samples. The conjugation of the different mal-PEG molecules on the —SH groups of the side chains of the Cys present in the ISVD-derived building blocks of the different molecules (6×5 kDa, 5×5 kDa, 4×5 kDa and 1×30 kDa, see FIG. 25) was confirmed.

### Half-Life Extension Driven by Hydrodynamic Radius

The results indicate that the hydrodynamic radius of the boxed analytes exceeds renal clearance (approx. 60 kDa) and should not differ significantly in HLE. A DOL of 6×5 kDa resulted in a greater hydrodynamic size (apparent MW of 105 kDa) compared to a DOL1 of 30 kDa (apparent MW of 97 kDa). See FIG. 25.

### Rat Plasma PK of ISVD-Carrier Compounds

The following constructs were tested: CARGO-conjugated T028100075-5 kDaPEG (1×), T028100075-30 kDaPEG (1×) and T028100075-Ala (1×), as described above.

In addition, ISVD-based building block with SEQ ID NO.: 86, which comprises 6 Cys located at solvent-accessible positions, comprised in molecule T028100078 (SEQ ID NO.: 113), was conjugated to 5 kDaPEG (6×). The molecule was generated following the same procedure as described above.

### Protocol

### Rat In Vivo Study

The PK assessment was performed in female Wistar rats (Charles River Laboratories, Sulzfeld, Germany), 12 weeks of age were acclimatized to laboratory conditions for a 7-day period prior to the commencement of the study. On the morning of dosing, the required number of aliquots of the test item stock solutions were thawed at +25° C. using a water bath and swirled gently for 5-10 minutes. Stock solutions were diluted in the appropriate volume of commercial and sterile D-PBS under laminar flow. IRR-VHH solution (1.5 mg/mL) was administered to the appropriate animals via a single intravenous (bolus) injection at 3 mg/kg on day 0, in the morning.

Blood was collected at different timepoints (0.083 h, 1 h, 3 h, 6 h, 24 h, 48 h, 96 h, 168 h for T028100075-30 kDaPEG and T028100078-5 kDaPEG (6×) ISVD compounds, 0.083 h, 1 h, 3 h, 6 h, 24 h, 48 h, 72 h for T028100075-5 kDaPEG and T028100075-Ala ISVD compounds). The animals were restrained during blood sampling for intermediate sampling. Blood (200 μl per sample) from the saphenous vein was collected in labelled 200 μL non-anticoagulated poly (propylene) micro tubes (Sarstedt, Microvette® 200 Z, cat #20.1290). Immediately following collection, blood samples were slowly homogenized and stored at room temperature. For terminal sampling, blood was collected via axillary cut down following isoflurane inhalation anesthesia. Blood samples were collected into labelled 4.5 mL non-anticoagulated poly (propylene) tubes (Sarstedt, cat #32.329)., and placed on the benchtop at room temperature for 30 minutes. The samples were then centrifugated at 4° C. for 10 min at 2000 g, and the supernatant (serum) apportioned into 50 μL aliquots in clean non-anticoagulated poly (propylene) 0.5 ml tubes (Brand, Micro tube, cat #780760) and stored at −80° C. Each sample was subject to one freeze thaw cycle prior to analysis.

Urine samples were collected at 0-8 h, 8-24 h, 24-48 h and 48-72 h intervals from metabolic cages (Föhr Medical Instruments, SWK). Urine samples were apportioned in labelled 30 ml type tubes (Sarstedt, cat #62.555), and stored at −80° C. until analysis.

In vivo experiments were conducted according to the policy of the Federation of European Laboratory Animal Science Associations and the European Convention for the protection of vertebrate animals used for experimental and other scientific purposes, with the implementation of the principles of the 3Rs (replacement, reduction, and refinement). In vivo studies were conducted in compliance with the Sanofi institutional animal care policy.

### Measurement of ISVD-Based Carrier Compound Concentrations in Rat Serum.

Total PK assay in rat serum for PEG-conjugated T028100075-5 kDaPEG and T028100075-Ala molecules

A streptavidin-coated MSD GOLD 96-well SMALLSPOT® plate (Meso Scale Discovery, cat #L45SA) was blocked with SuperBlock™ blocking buffer (ThermoFisher Scientific, cat #37515) for 30 minutes at RT. The plate was then washed with PBS and incubated for 1 hour at RT and at 600 rpm with 1.0 μg/mL biotinylated ABH0190 monoclonal antibody (mAb) (Sanofi proprietary antibody), directed against the framework of the different ISVD building blocks. Calibrators and Quality Controls (QCs) were prepared in pooled rat serum. After washing, calibrators, QCs and study samples were applied at a minimum required dilution (MRD) of 20 in PBS/0.1% casein (Biorad, cat #161-0783) and incubated for 1 hour at RT and at 600 rpm. After washing the plate was incubated for 1 hour at room temperature (RT) and at 600 rpm with 2.0 μg/mL sulfo-labelled ABH0085 mAb (Sanofi proprietary antibody), specifically directed against the half-life extension building block ALB23 of ISVD compounds.

After washing, MSD Read buffer A (Meso Scale Discovery, cat #R92TG) was added and ECL values were measured with a Sector Imager Quickplex SQ 120 (Meso scale Discovery). Calibration curve responses were processed using a 4PL-1/Y² weighted fir (with log (X) transformation) of electrochemiluminescence (ECL) responses versus concentrations. The concentrations of calibrators, QC and study samples (reported values) were calculated by interpolation based on the fit of the calibration curve.
Total PK assay in rat serum for PEG-conjugated T028100075-30 kDaPEG and T028100078-5 kDaPEG (6×) molecules

A Quickplex 96-well High Bind Plate (Meso Scale Discovery, cat #L55XB) was coated for 1 hour at RT with 3.0 μg/mL ABH0190 mAb (Sanofi proprietary antibody). The plate was then aspirated and blocked with 1% BSA (bovine serum albumin; Sigma Aldrich; cat #A3059) for 30 minutes at RT. Calibrators and QCs were prepared in pooled rat serum. After washing, calibrators, QCs and study samples were applied at a MRD of 100 in PBS/0.1% casein (Biorad, cat #161-0783) and incubated for 3 hours at RT and at 600 rpm. After washing, the plate was incubated for 1 hour at RT and at 600 rpm with 5.0 g/mL of biotinylated anti-PEG mAb (IBMS Sinica, cat #6.3-PABG-B), which binds to the backbone of PEG moiety of the different ISVD compounds. After washing, the plate was incubated for 1 hour at RT and 600 rpm with 0.5 g/mL of Sulfo-tag labeled Streptavidin (Meso Scale Discovery, cat #R32AD).

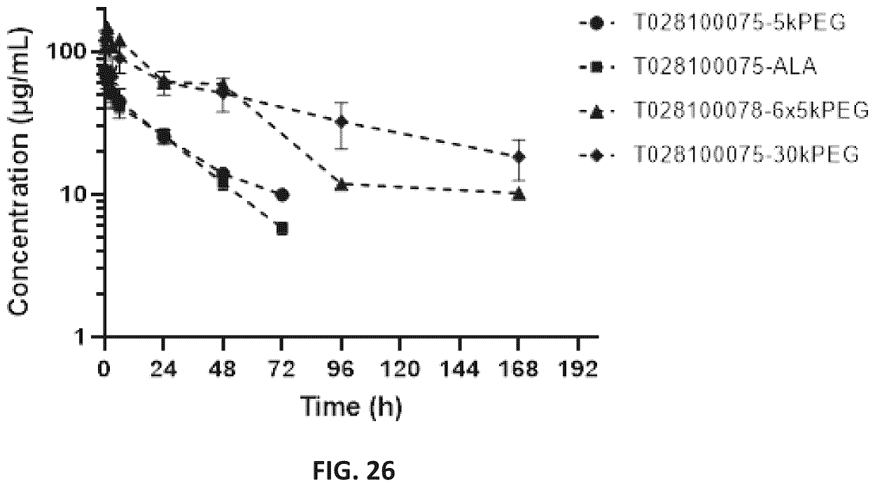

After washing, MSD Read buffer A (Meso Scale Discovery, cat #R92TG) was added and ECL values were measured with a Sector Imager Quickplex SQ 120 (Meso scale Discovery). Calibration curve responses were processed using a 5PL-1/Y2 weighted fir (with log (X) transformation) of electrochemiluminescence (ECL) responses versus concentrations. The concentrations of calibrators, QC and study samples (reported values) were calculated by interpolation based on the fit of the calibration curve. Results are shown in FIG. 26. As shown in this figure, and as expected, no significant differences in half-life were observed for the molecules comprising 6×5 kDa PEG and 1×30 kDa PEG. PK parameter estimates were determined from individual animal data using non-compartmental analysis in WinNonlin (Phoenix version 8.2) using the plasma data module with linear up log down interpolation.

### Example 16. Non-Target Specificity Profiling of Carriers Using a Membrane Proteome Array (MPA)

As discussed above, the protein-based building blocks of the present technology do not bind any protein or other biological compound (biomolecule), in particular they do not bind any human protein. With binding-FACS experiments, used to study cell binding and internalization of loaded ISVD-Carrier constructs, it was demonstrated that the protein-based building blocks according to the present technology (in particular, the ones used in the present examples, such as the ones used in Examples 11-14) do not specifically bind to any of the cell lines used in the examples: K-562, HeLa, SK-OV3, NCI-H226 and BxPC-3. Table 18 shows the molecules comprising protein-based carrier building blocks of the present technology, which has been conjugated with Alanine, which have been tested for cell binding (the tested cells are also disclosed in Table 18).

TABLE 18

| | Maleimide ALA-conjugated | |
|---|---|---|
| | protein-based building block | |
| Cell line | comprising molecule | SEQ ID |
| K-562 | T028100069, T028100070 | 107 and 108 |
| HeLa | T028100069, T028100070 | 107 and 108 |
| SK-OV3 | T028100069, T028100070 | 107 and 108 |
| NCl-H226 | ALB-3C_K27m_w1 | 173 |
| | ALB-5C_K27m | 174 |
| BxPC-3 | ALB-3C_K27m_w1 | 173 |
| | ALB-5C_K27m | 174 |
| | T028100078 | 113 |
| | ALB-3C_hCKS1_c3 | 125 |

In addition, 3 different protein-based building blocks were produced, their engineered cysteines (Cys present at solvent-accessible positions in the building blocks) were blocked using maleimide-Alanine and their non-target binding was tested via the Membrane Proteome Array™ (https://www.integralmolecular.com/membrane-proteome-array/). This cell-based array contains one of the largest set of human membrane proteins (including heterocomplexes) assembled to determine specificity and preclinical safety of proteins such as antibodies, CAR-T cell therapies, and other biotherapeutics, see, e.g., Tucker DF., et al., “Isolation of state-dependent monoclonal antibodies against the 12-transmembrane domain glucose transporter 4 using virus-like particles”, 2018, 115 (22) E4990-E4999.

The 3 selected carrier molecules were T028100069 (SEQ ID NO.: 107), ALB-3C_hCKS1_c3 (SEQ ID NO.: 125) and ALB-3C_K27m_w1 (SEQ ID NO.: 173), comprising an ISVD-based building block (SEQ ID NO.: 80), a human protein-based building block (SEQ ID NO.: 101) and a DARPin-based building block (SEQ ID NO.: 97), respectively.

The assay consisted of screening 6,000 human membrane proteins (>5,300 unique) which are natively expressed in unfixed human cells in a 384-well plate format. Assay read out was done via sensitive flow cytometry detection and an anti-VHH secondary detection reagent from Jackson ImmunoResearch (Cat #128-605-232) was used. As the above molecules all comprise a serum albumin binding VHH, excess human serum albumin was present to exclude any albumin interactions. The screening of the three selected carrier molecules did not generate positive signals and only background signals were observed.

### Example 17. Solubility of Molecule T028100070 (SEQ ID NO.: 108)

Before Size Exclusion Chromatography as formulation step, 1.4 gram of purified T028100070 molecule (purified via ProteinA Amphere A3 (JSR) and Capto Q Impress (Cytiva), was concentrated using a 10 kDa MWCO Vivaflow cassette, a disposable and ready-to-use crossflow device (Sartorius), to a final concentration of 35.9 mg/ml in PBS+0.1 mM TCEP, at RT. The molecule was soluble at this concentration.

### Example 18. Conjugation of Different Cargos on to a Single Protein-Based Building Block

To confirm the feasibility of different cargo conjugation on to a single protein-based building block, the following construct was generated: EGFR7D12-3C_hCKS1_c3-cMyc NLS (SEQ ID NO.: 215):

DVQLEESGGGSVQTGGSLRLTCAASGRTSRSYGMGWFRQAPGKEREFVS
GISWRGDSTGYADSVKGRFTISRDNAKNTVDLQMNSLKPEDTAIYYCAA
AAGSAWYGTLYEYDYWGQGTQVTVSSGGGGSGGGGSGGGGSHKQIYYSD
KCDDEEFEYRHVMLPKDIAKLVPKTHLMSESEWRNLGVQQSCGWVHYMI
HEPEPHILLFRRPLPKKPKCGGGPAAKRVKLD

This construct comprises an epidermal growth factor receptor (EGFR)-binding VHH (SEQ ID NO.: 216):

DVQLEESGGGSVQTGGSLRLTCAASGRTSRSYGMGWFRQAPGKEREFVS
GISWRGDSTGYADSVKGRFTISRDNAKNTVDLQMNSLKPEDTAIYYCAA
AAGSAWYGTLYEYDYWGQGTQVTVSS

It also comprises a linker (GGGGGGGGSGGGGS, SEQ ID NO.: 163) and a nuclear localization sequence (cMyc NLS, SEQ ID NO.: 221, see also Day AH. et al., “Targeted cell imaging properties of a deep red luminescent iridium (iii) complex conjugated with a c-Myc signal peptide”, Chem Sci., 2020, 11 (6): 1599-1606) preceded by 3 Gly residues (GGGPAAKRVKLD, SEQ ID NO.: 217). The protein-based building block is a CKS-based building block (SEQ ID NO.: 101).

EGFR7D12-3C_hCKS1_c3-cMyc NLS was produced via Pichia in shake flasks. Cysteine capping was carried out during fermentation via cysteamine addition. Spent medium was harvested via centrifugation and DTT and PMSF were added at 10 mM and 1 mM respectively. Buffer exchange was carried out via TFF to 20 mM Acetate with 10 mM DTT, pH 5.0; this buffer was used as loading buffer for cation exchange chromatography on Capto S (Cytiva). The eluted material was further purified via size exclusion chromatography (SEC) on Supedex75, equilibrated in D-PBS+0.1 mM TCEP. QC via Mass Spec confirmed intact product.

Site-specific conjugation of maleimide-CMA-1 on the —SH group present on the side chain of solvent-accessible cysteines and stochastic conjugation of pHAb and Alexa 647 on the primary amine present in the side chain of lysines was performed.

CMA-1 is a cationic cell penetrating peptide (CPP) which is adopting its active conformation in acidic conditions (see, e.g., Yang Y. et al., “Application of peptides in construction of nonviral vectors for gene delivery”, Nanomaterials (Basel), 2022, 12 (22): 4076). This peptide has the following sequence: GGGIGAVLEVLTTGLPALISWIEEEEQQ (SEQ ID NO.: 218). The peptide was custom synthesized via solid phase synthesis with a maleimide group on the amino terminus for conjugation purpose and an amidated C-terminus for higher potency: Maleimide-GGGIGAVLEVLTTGLPALISWIEEEEQQ-NH₂ (SEQ ID NO.: 220).

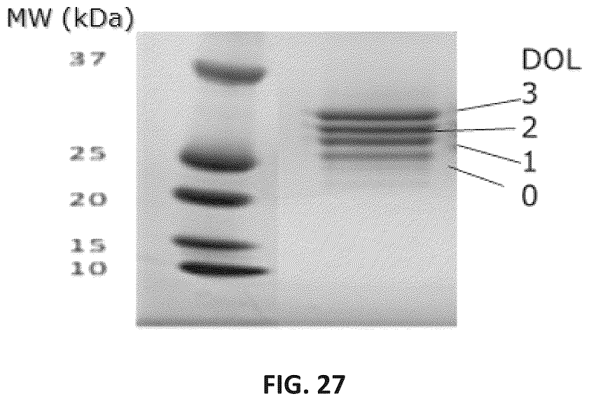

A limited CPP conjugation was carried out using a 1.2 molar ratio/free thiol of maleimide-CMA-1, resulting in a mixed population of CMA1 on the 3Cysteine CKS carrier (SEQ ID NO.: 101). A wide range of DOL was obtained ranging from 0 to 3. The remaining free thiols were blocked with an excess of maleimide-Alanine. The loaded carrier construct was analyzed via SDS-PAGE; result is shown in FIG. 27.

Next the CMA-1-loaded carrier was labeled with Alexa 647 and pHAb for tracking the molecule in cell-based assays. A 6 times molar excess of both NHS-dyes was added to the carrier in D-PBS+0.1 mM TCEP and incubated for 1 hr at room temperature. After 1 hr of incubation, another 6 times molar excess of both NHS-dyes was added, followed by an additional hour of incubation at room temperature. The excess dye was removed via SEC and the material was collected in D-PBS buffer.

Protein concentration and DOL of the fluorophores was determined via Nanodrop Spectrophotometer (Thermo Scientific), using the Proteins & Labels application module. This module displays the UV spectrum, measures the protein's absorbance at 280 nm (A280) (protein absorbance at 280 nm minus absorbance at 340 nm), measures the fluorochromes absorbance at Amax and calculates the concentration of the labeled antibody or protein (mg/mL) and of the fluorochrome (UM). The extinction coefficient of the protein was added and the fluorochromes were selected in the Dye 1 and Dye2 box; the Amax was set automatically for the two fluorochromes. See Table 19.

TABLE 19

| | | | | Molar extinction |
|---|---|---|---|---|
| | MW | ME | | coefficient |
| Label | (Da) | (DOL = 1-2) | ODmax | (M⁻¹ cm⁻¹) |
| Alexa647 | 1250 | 3 | 650 | 239000 |
| pHAb | 822 | 10-20 | 532 | 75000 |

The spectrum automatically gave the maximum absorbance values in the scanned range (200 nm to 750 nm), the normalized absorbance at 280 nm and the calculated protein concentration (mg/ml). The DOL was calculated as [ODmax*Mw Protein (Da)]/[conc labeled protein (mg/ml)*Molar extinction coefficient Label (M−1 cm−1)].

The result is shown in Table 20. Both for pHAb and Alexa647 a DOL2 and DOL3 on the CMA1 loaded carrier and control construct was obtained, respectively.

TABLE 20

| | Conc | Volume | DOL | DOL |
|---|---|---|---|---|
| ISVD carrier construct | (mg/ml) | (ml) | A647 | pHAb |
| EGFR7D12-3C_hCKS1_c3- | 0.439 | 0.75 | 2.073 | 2.107 |
| cMyc NLS + CMA1 | | | | |
| EGFR7D12-3C_hCKS1_c3- | 0.308 | 0.5 | 3.096 | 3.126 |
| cMyc NLS + Ala | | | | |

The conjugations which were carried out on both engineered cysteines (site-specific conjugation) and surface exposed lysines (stochastic conjugation) shows the feasibility of conjugating different cargos onto the protein-based building blocks of the present technology. Here we demonstrate that conjugation in one single building block with four different cargos. In particular, it has been demonstrated that a single protein-based carrier building block can comprise different types of attachment points or conjugation sites (e.g., in this case, the primary amine from the N-terminus, primary amines comprised in the side chain of Lys comprised in the protein-based building block and —SH groups comprised in the side chain of Cys comprised in the protein-based building block. Four different cargos (EGFR-binding ISVD (SEQ ID NO.: 216), two different dyes (pHAb and Alexa647) and CMA-1 peptide (SEQ ID NO.: SEQ ID NO.: 218). Each cargo has been specifically conjugated to one type of attachment point or conjugation site. The EGFR-binding ISVD is conjugated to the N-terminus primary amine of the carrier building block. The CMA-1 peptide is conjugated to the SH-group comprised in the side chain of Cys comprised in the protein-based building blocks. Two different dyes, pHAb and Alexa647, are conjugated to the primary amine comprised in the side chain of Lys comprised in the protein-based building block.

### Example 19. Generation of ISVD-Based Building Block Comprising 9 Cys at Solvent Accessible Positions

The ISVD precursor was RSV001A04, SEQ ID NO.: 179:
EVQLVESGGGLVQAGGSLSISCAASGGSLSNYVLGWFRQAPGKEREFVA
AINWRGDITIGPPNVEGRFTISRDNAKNTGYLQMNSLAPDDTAVYYCGA
GTPLNPGAYIYDWSYDYWGRGTQVTVSS

Starting from this precursor, the following ISVD-derived building blocks are generated, as described in detail in Example 1:

19001
(SEQ ID NO.: 222)
EVQLVESGGGLVQAGGSLSICCAASGGSLSCYVLGWFRQAPGCEREFVA
AINWRGDITIGPPNVEGRFCISRCNAKNTGYLQMNCLAPDDTAVYYCGA
GTPLNPCAYIYCWSYDYWGCGTLVTVSS
19002
(SEQ ID NO.: 223)
EVQLVECGGGLVCAGGSLCISCCASGGSLSNYVLGWFRQAPGKCREFVA
AINWRGCITIGPPCVEGRFTICRDNCKNTGYLQMNSLAPDDTAVYYCGA
GTPLNPGAYIYDWSYDYWGRGTLVTVSS
19003
(SEQ ID NO.: 224)
EVQLVECGGGLVQAGGCLSISCAASGGSLSCYVLGWFRQAPGKCREFVA
AINWRGCITIGPPCVEGRFCISRDNACNTGYLQMNSLAPDDTAVYYCGA
GTPLNPGAYIYDWSYDYWGRGTLVTVCS

These building blocks comprise 9 Cys located at solvent-accessible positions, which SH-groups are attachment points or conjugation sites for site-directed conjugation of cargos.

TABLE A-1
Preferred Linker sequences

| | SEQ ID | |
|---|---|---|
| Name of linker | NO: | Amino acid sequences |
| GS5 (5GS) | 158 | GGGGS |
| GS7 (7GS) | 159 | SGGSGGS |
| GS8 (8GS) | 160 | GGGGGGGS |
| GS9 (9GS) | 161 | GGGGSGGGS |
| GS10 (10GS) | 162 | GGGGSGGGGS |
| GS15 (15GS) | 163 | GGGGSGGGGSGGGGS |
| GS18 (18GS) | 164 | GGGGSGGGGSGGGGGGGS |
| GS20 (20GS) | 165 | GGGGSGGGGSGGGGSGGGGS |
| GS25 (25GS) | 166 | GGGGSGGGGSGGGGSGGGGSGGGGS |
| GS30 (30GS) | 167 | GGGGSGGGGSGGGGSGGGGSGGGGSGGGGS |
| GS35 (35GS) | 168 | GGGGSGGGGSGGGGSGGGGSGGGGSGGGGSGGGG |
| | | S |
| GS40 (40GS) | 169 | GGGGSGGGGSGGGGGGGGSGGGGSGGGGSGGGG |
| | | SGGGGS |
| A3 (3A) | — | AAA |
| G1 hinge | 193 | EPKSCDKTHTCPPCP |
| 9GS-G1 hinge | 194 | GGGGSGGGSEPKSCDKTHTCPPCP |
| Llama upper long hinge | 195 | EPKTPKPQPAAA |
| region | | |
| G3 hinge | 196 | ELKTPLGDTTHTCPRCPEPKSCDTPPPCPRCPEPKSCDTP |
| | | PPCPRCPEPKSCDTPPPCPRCP |

### ITEMS OF THE PRESENT TECHNOLOGY

The present technology provides the following items:
1. A molecule comprising at least one protein-based building block, wherein the at least one protein-based building block:
   a) comprises at least one, preferably at least two conjugation sites or attachment points;
   b) has a molecular mass of about 2.5 to about 70 kDa, preferably of about 2.5 to about 50 kDa, or of about 2.5 to less than 50 kDa, more preferably of about 2.5 to about 30 kDa, even more preferably of about 2.5 to about 16 kDa;
   c) has a globular three-dimensional (3D) structure;
   d) has a solubility of 10 mg/ml or more, preferably of 20 mg/mL, preferably of 50 mg/ml or more, and even more preferably of 100 mg/ml, measured in an aqueous solution at room temperature, preferably wherein the aqueous solution is citrate buffer 5 mM or PBS, at pH 7.0 or 7.4;
   e) does not specifically bind to any human protein or binds one or more human proteins with a K_{D} (K_{D} value) greater than 5×10⁻⁴ mol/litre, preferably as determined by cell-binding assay and/or by surface plasmon resonance (SPR), for instance as described in Ober et al. 2001, Intern. Immunology 13:1551-1559, or does not specifically bind to any human cell or binds one or more human cells with a K_{D} (K_{D} value) greater than 5×10⁻⁴ mol/litre, preferably as determined by cell-binding assay or by SPR;
   f) optionally, does not specifically bind to any (non-human) molecule which the protein-based carrier building block precursor specifically binds to, such as protein F of RSV, or binds to any (non-human) molecule which the protein-based carrier building block precursor specifically binds to, such as protein F of RSV, with a K_{D} (K_{D} value) greater than 5×10⁻⁴ mol/litre, preferably as determined by cell-binding assay or by SPR;
   g) optionally, does not specifically bind to any human cell and/or cell type, or binds to a human cell and/or cell type with a K_{D} (K_{D} value) greater than 5×10⁻⁴ mol/litre, preferably as determined by cell-binding assay;
   h) optionally, does not specifically bind any microorganism such as bacteria, fungi, protists, yeast and/or to any virus, or binds to a microorganism such as bacteria, fungi, protists, yeast and/or to virus with a K_{D} (K_{D} value) greater than 5×10⁻⁴ mol/litre, preferably as determined by cell-binding assay and/or SPR, as described herein;
   i) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein;
   j) optionally, does not specifically bind to any biomolecule, including human biomolecules and non-human biomolecules, such as plant biomolecules, virus biomolecules and/or microorganism biomolecules (such as bacteria, fungi, protists and/or yeast), or binds to biomolecules, including human biomolecules and non-human biomolecules, with a K_{D} (K_{D} value) greater than 5×10⁻⁴ mol/litre, preferably as determined by cell-binding assay and/or SPR, as described herein when it has at least one cargo attached to it (via the at least one conjugation sites or attachment points comprised therein);
   k) optionally, does not comprise or consists of an amino acid sequence selected from SEQ ID NO.: 1-34 as depicted on Tables A-1 and A-2 of WO 2016/055656 and/or SEQ ID NO.: 1-12 as depicted on Table A-1 of WO 2010/139808; and
   l) optionally, does not comprise or consists of the amino acid sequence as defined in SEQ ID NO.: 214.
2. The molecule of item 1, wherein the at least one, preferably the at least two conjugation site(s) or attachment point(s) are present at a solvent-accessible positions in the protein-based building block.
3. The molecule of any of items 1 or 2, wherein the at least one, preferably the at least two attachment point(s) or conjugation site(s) are reactive groups present in the side chain of any amino acid in the protein-based carrier building block, preferably an amino acid present at a solvent-accessible position in the protein-based carrier building block, more preferably are reactive groups present in the side chain of a cysteine and/or in the side chain of a tyrosine, and/or in the side chain of a lysine, and/or in the side chain of a non-natural amino acid, preferably located at solvent-accessible positions in the protein-based carrier building block.
4. The molecule of any of items 1 to 3, wherein at least one of the attachment point(s) or conjugation site(s), preferably at least two, more preferably all of the attachment points or conjugation sites, is an engineered attachment point or conjugation site.
5. The molecule of any of items 1 to 4, wherein the at least one protein-based building block and/or the molecule do not specifically bind crystallizable fragment (Fc) receptors (FcRs),
Fc-binding proteins and/or Fc-sensors (or bind FcRs, Fc-binding proteins and/or Fc-sensors with a K_{D} value greater than 5×10⁻⁴ mol/litre).
6. The molecule of any of items 1 to 5, wherein the at least one protein-based building block and/or molecule does not show effector functions of conventional antibodies mediated by the Fc domain.
7. The molecule of any of items 1 to 6, wherein the at least one protein-based building block does not specifically bind the variable domain of the light chain (V_{L}) and/or the variable domain of the heavy chain (V_{H}) of an antibody, such as the V_{L} and/or the V_{H} of a monoclonal antibody (mAb).
8. The molecule of any of items 1 to 7, wherein the at least one protein-based building block does not specifically bind the first constant domain of the heavy chain (C_{H}1) of an antibody, such as the C_{H}1 of a mAb, and/or does not specifically bind the constant domain of the light chain (C_{L}) of an antibody, such as the C_{L} of a mAb; and/or does not specifically bind the third constant domain of the heavy chain (C_{H}3) of an antibody, such as the C_{H}3 of a mAb, and/or does not specifically bind the second constant domain of the heavy chain (C_{H}2) of an antibody, such as the C_{H}2 of a mAb.
9. The molecule of any of items 1 to 8, wherein the at least one protein-based building block does not specifically bind to any non-protein molecule, such as DNA, RNA, lipids (e.g., such as phosphatidylserine (PS)) or glycans, or binds one or more non-protein molecules with a K_{D} value greater than 5×10⁻⁴ mol/litre.
10. The molecule of any of items 1 to 9, wherein the at least one protein-based building block does not specifically bind the precursor's target.
11. The molecule of any of items 1 to 10, wherein the at least one protein-based building block comprises at least one cargo attached to at least one attachment point or conjugation site.
12. The molecule of any of items 1 to 11, wherein the at least one protein-based building block is not derived from the crystallizable fragment of an antibody such as the Fc fragment of a mAb, and/or is not derived from the CH₂ and/or the CH₃ domains of the Fc fragment, and/or is not derived from a CH₁ and/or the C_{L} domains comprised in the antigen-binding fragment (Fab) of an antibody, such as the CH₁ and/or the C_{L} domains comprised in the Fab of a mAb.
13. The molecule of any of items 1 to 12, wherein the molecule is not (or is not derived from) a crystallizable fragment (Fc) of an antibody, such as a mAb and/or is not (or is not derived from) the Fab of an antibody, such as a mAb.
14. The molecule of any of items 1 to 13, wherein, if there is more than one attachment point or conjugation site in the at least one protein-based building block, the conjugation sites are spatially distant from each other.
15. The molecule of any of items 1 to 14, wherein the at least one, preferably two conjugation site(s) is (are) selected from a primary amine, a thiol group, a hydroxyl group, a guanidino group, a carboxyl group and/or a thioether group, preferably from a primary amine and/or a thiol group, more preferably a thiol group.
16. The molecule of any of items 1 to 15, wherein the at least one protein-based carrier building block comprises at least two engineered cysteines, preferably located at solvent accessible positions, such as three engineered cysteines, or six engineered cysteines, or nine engineered cysteines, preferably located at solvent accessible positions, with free or capped thiol groups at their side chains, that are the at least two, such as three, or six, or nine, conjugation sites or attachment points.
17. The molecule of any of items 1 to 16, wherein the at least one protein-based carrier building block comprises at least three engineered cysteines with free or capped thiol groups at their side chain, and at least three lysines with free or capped amine groups at their side chain, preferably located at solvent accessible positions, or wherein the at least one protein-based carrier building block comprises at least two engineered lysines with free or capped amine groups at their side chain, preferably located at solvent accessible positions, and optionally or additionally a free N-terminal amine.
18. The molecule of any of items 1 to 17, wherein the at least one protein-based building block comprises a N- and/or a C-terminal Cys and/or a N- and/or a C-terminal Tyr, preceded or followed by a (GG) or (G₄S₁)₁₋₃GG sequence, such as CGG-, -GGC, YGG-, -GGY, -(G₄S₁)₁₋₃GGY, Y(G₄S₁)₁₋₃GG-, YGG(S₁G₄)₁₋₃-, or YGG(G₄S₁)₁₋₃-.
19. The molecule of any of items 1 to 18, wherein the protein-based building block is a small globular non-human protein-based building block or a small globular human protein-based building block.
20. The molecule of item 19, wherein the small globular non-human protein-based building block is an immunoglobulin single variable domain (ISVD)-based building block, a DARP-in-based building block, an affibody-based building block or an affitin-based building block.
21. The molecule of item 19, wherein the small globular human protein-based building block is derived from cyclin-dependent kinase subunit 1 (CKS1) protein.
22. The molecule of any of items 19 to 20, wherein the ISVD-based building block is derived from a V_{H}, a humanized V_{H}, a human V_{H}, a V_{HH}, a humanized V_{HH} or a camelized V_{H} (derived from a heavy-chain ISVD).
23. The molecule of item 22, wherein the ISVD-based building block is derived from an ISVD belonging to the “V_{H}3 class”.
24. The molecule of any of items 19 to 20 or 22 to 23, wherein, in the ISVD-derived protein-based carrier building block, the amino acid at position 11 (according to Kabat) is Val or Leu, preferably Val, and/or the amino acid at position 89 (according to Kabat) is Val, Thr or Leu, preferably Leu; and/or the amino acid at position 108 is a Leu or Gln, preferably Leu; and/or the amino acid at position 110 (according to Kabat) is Thr, Lys or Gln, preferably Thr; and/or the amino acid at position 112 (according to Kabat) is Ser, Lys or Gln, preferably Ser; and/or the ISVD-based building block contains a C-terminal extension of 1-5 amino acids chosen from any naturally occurring amino acid, preferably chosen from Ala, Gly and/or Cys.
25. The molecule of any of items 19 to 20 or 22 to 24, wherein the ISVD-derived building block is derived from RSV001A04 (SEQ ID NO.: 179).
26. The molecule of any of items 19 to 20 or 22 to 25, wherein the ISVD-derived building block comprises or, alternatively, consists of SEQ ID NO.: 186:

X₁VX₂LX₃EX₄X₅GX₆X₇X₈X₉X₁₀X₁₁GX₁₂X₁₃X₁₄₁X₁₅CX₁₆AX₁₇X₁₈X₁₉X₂₀L
X₂₁X₂₂X₂₃VLGWFRX₂₄AX₂₅X₂₆X₂₇X₂₈X₂₉X₃₀FVAAINX₃₁X₃₂X₃₃X₃₄X₃₅
X₃₆X₃₇X₃₈PX₃₉X₄₀VX₄₁X₄₂X₄₃FX₄₄₁X₄₅X₄₆X₄₇X₄₈X₄₉X₅₀X₅₁TGX₅₂LX₅₃M
X₅₄X₅₅LX₅₆X₅₇X₅₈DX₅₉AX₆₀YX₆₁CGAGX₆₂PX₆₃X₆₄X₆₅X₆₆AYX₆₇X₆₈X₆₉
X₇₀SYX₇₁X₇₂X₇₃GX₇₄X₇₅TX₇₆VX₇₇VX₇₈X₇₉X₈₀X₈₁X₈₂

wherein
X₁ (position 1 according to Kabat numbering) can be Glu or any amino acid with a reactive group in its side chain, such as cysteine;
X₂ (position 3 according to Kabat numbering) can be Gln or any amino acid with a reactive group in its side chain, such as cysteine;
X₃ (position 5 according to Kabat numbering) can be Val or any amino acid with a reactive
X₄ (position 7 according to Kabat numbering) can be Ser or any amino acid with a reactive group in its side chain, such as cysteine;
X₅ (position 8 according to Kabat numbering) can be Gly or any amino acid with a reactive group in its side chain, such as cysteine;
X₆ (position 10 according to Kabat numbering) can be Gly or any amino acid with a reactive group in its side chain, such as cysteine;
X₇ (position 11 according to Kabat numbering) can be Leu, Val Ser, Met, Trp, Phe, Thr, Gln, Glu, Ala, Arg, Gly, Lys, Tyr, Asn, Pro or Ile, preferably Leu or Val or any amino acid with a reactive group in its side chain, such as cysteine;
X₈ (position 12 according to Kabat numbering) can be Val or any amino acid with a reactive group in its side chain, such as cysteine;
X₉ (position 13 according to Kabat numbering) can be Gln or any amino acid with a reactive group in its side chain, such as cysteine;
X₁₀ (position 14 according to Kabat numbering) can be Ala or any amino acid with a reactive group in its side chain, such as cysteine;
X₁₁ (position 15 according to Kabat numbering) can be Gly or any amino acid with a reactive group in its side chain, such as cysteine;
X₁₂ (position 17 according to Kabat numbering) can be Ser or any amino acid with a reactive group in its side chain, such as cysteine;
X₁₃ (position 18 according to Kabat numbering) can be Leu or any amino acid with a reactive group in its side chain, such as cysteine;
X₁₄ (position 19 according to Kabat numbering) can be Ser or any amino acid with a reactive group in its side chain, such as cysteine;
X₁₅: (position 21 according to Kabat numbering) can be Ser or any amino acid with a reactive group in its side chain, such as cysteine;
X₁₆: (position 23 according to Kabat numbering) can be Ala or any amino acid with a reactive group in its side chain, such as cysteine;
X₁₇: (position 25 according to Kabat numbering) can be Ser or any amino acid with a reactive group in its side chain, such as cysteine;
X₁₈: (position 26 according to Kabat numbering) can be Gly or any amino acid with a reactive group in its side chain, such as cysteine;
X₁₉: (position 27 according to Kabat numbering) can be Gly or any amino acid with a reactive group in its side chain, such as cysteine;
X₂₀: (position 28 according to Kabat numbering) can be Ser or any amino acid with a reactive group in its side chain, such as cysteine;
X₂₁: (position 30 according to Kabat numbering) can be Ser or any amino acid with a reactive group in its side chain, such as cysteine;
X₂₂: (position 31 according to Kabat numbering) can be Asn or any amino acid with a reactive group in its side chain, such as cysteine;
X₂₃: (position 32 according to Kabat numbering) can be Tyr or any amino acid with a reactive group in its side chain, such as cysteine;
X₂₄: (position 39 according to Kabat numbering) can be Gln or any amino acid with a reactive group in its side chain, such as cysteine;
X₂₅: (position 41 according to Kabat numbering) can be Pro or any amino acid with a reactive group in its side chain, such as cysteine;
X₂₆: (position 42 according to Kabat numbering) can be Gly or any amino acid with a reactive group in its side chain, such as cysteine;
X₂₇: (position 43 according to Kabat numbering) can be Lys or any amino acid with a reactive group in its side chain, such as cysteine;
X₂₈: (position 44 according to Kabat numbering) can be Glu or any amino acid with a reactive group in its side chain, such as cysteine;
X₂₉: (position 45 according to Kabat numbering) can be Arg or any amino acid with a reactive group in its side chain, such as cysteine;
X₃₀: (position 46 according to Kabat numbering) can be Glu or any amino acid with a reactive group in its side chain, such as cysteine;
X₃₁: (position 52a according to Kabat numbering) can be Trp or any amino acid with a reactive group in its side chain, such as cysteine;
X₃₂: (position 53 according to Kabat numbering) can be Arg or any amino acid with a reactive group in its side chain, such as cysteine;
X₃₃: (position 54 according to Kabat numbering) can be Gly or any amino acid with a reactive group in its side chain, such as cysteine;
X₃₄: (position 55 according to Kabat numbering) can be Asp or any amino acid with a reactive group in its side chain, such as cysteine;
X₃₅: (position 56 according to Kabat numbering) can be Ile or any amino acid with a reactive group in its side chain, such as cysteine;
X₃₆: (position 57 according to Kabat numbering) can be Thr or any amino acid with a reactive group in its side chain, such as cysteine;
X₃₇: (position 58 according to Kabat numbering) can be Ile or any amino acid with a reactive group in its side chain, such as cysteine;
X₃₈: (position 59 according to Kabat numbering) can be Gly or any amino acid with a reactive group in its side chain, such as cysteine;
X₃₉: (position 61 according to Kabat numbering) can be Pro or any amino acid with a reactive group in its side chain, such as cysteine;
X₄₀: (position 62 according to Kabat numbering) can be Asn or any amino acid with a reactive group in its side chain, such as cysteine;
X₄₁: (position 64 according to Kabat numbering) can be Glu or any amino acid with a reactive group in its side chain, such as cysteine;
X₄₂: (position 65 according to Kabat numbering) can be Gly or any amino acid with a reactive group in its side chain, such as cysteine;
X₄₃: (position 66 according to Kabat numbering) can be Arg or any amino acid with a reactive group in its side chain, such as cysteine;
X₄₄: (position 68 according to Kabat numbering) can be Thr or any amino acid with a reactive group in its side chain, such as cysteine;
X₄₅: (position 70 according to Kabat numbering) can be Ser or any amino acid with a reactive group in its side chain, such as cysteine;
X₄₆: (position 71 according to Kabat numbering) can be Arg or any amino acid with a reactive group in its side chain, such as cysteine;
X₄₇: (position 72 according to Kabat numbering) can be Asp or any amino acid with a reactive group in its side chain, such as cysteine;
X₄₈: (position 73 according to Kabat numbering) can be Asn or any amino acid with a reactive group in its side chain, such as cysteine;
X₄₉: (position 74 according to Kabat numbering) can be Ala or any amino acid with a reactive group in its side chain, such as cysteine;
X₅₀: (position 75 according to Kabat numbering) can be Lys or any amino acid with a reactive group in its side chain, such as cysteine;
X₅₁: (position 76 according to Kabat numbering) can be Asn or any amino acid with a reactive group in its side chain, such as cysteine;
X₅₂: (position 79 according to Kabat numbering) can be Tyr or any amino acid with a reactive group in its side chain, such as cysteine;
X₅₃: (position 81 according to Kabat numbering) can be Gln or any amino acid with a reactive group in its side chain, such as cysteine;
X₅₄: (position 82a according to Kabat numbering) can be Asn or any amino acid with a reactive group in its side chain, such as cysteine;
X₅₅: (position 82b according to Kabat numbering) can be Ser or any amino acid with a reactive group in its side chain, such as cysteine;
X₅₆: (position 83 according to Kabat numbering) can be Ala or any amino acid with a reactive group in its side chain, such as cysteine;
X₅₇: (position 84 according to Kabat numbering) can be Pro or any amino acid with a reactive group in its side chain, such as cysteine;
X₅₈: (position 85 according to Kabat numbering) can be Asp or any amino acid with a reactive group in its side chain, such as cysteine;
X₅₉: (position 87 according to Kabat numbering) can be Thr or any amino acid with a reactive group in its side chain, such as cysteine;
X₆₀: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val or any other amino acid with a reactive group in its side chain, such as cysteine;
X₆₁: (position 91 according to Kabat numbering) can be Tyr or any amino acid with a reactive group in its side chain, such as cysteine;
X₆₂: (position 96 according to Kabat numbering) can be Thr or any amino acid with a reactive group in its side chain, such as cysteine;
X₆₃: (position 98 according to Kabat numbering) can be Leu or any amino acid with a reactive group in its side chain, such as cysteine;
X₆₄: (position 99 according to Kabat numbering) can be Asn or any amino acid with a reactive group in its side chain, such as cysteine;
X₆₅: (position 100 according to Kabat numbering) can be Pro or any amino acid with a reactive group in its side chain, such as cysteine;
X₆₆: (position100a according to Kabat numbering) can be Gly or any amino acid with a reactive group in its side chain, such as cysteine;
X₆₇: (position100d according to Kabat numbering) can be Ile or any amino acid with a reactive group in its side chain, such as cysteine;
X₆₈: (position100e according to Kabat numbering) can be Tyr or any amino acid with a reactive group in its side chain, such as cysteine;
X₆₉: (position 100f according to Kabat numbering) can be Asp or any amino acid with a reactive group in its side chain, such as cysteine;
X₇₀: (position 100g according to Kabat numbering) can be Trp or any amino acid with a reactive group in its side chain, such as cysteine;
X₇₁: (position 101 according to Kabat numbering) can be Asp or any amino acid with a reactive group in its side chain, such as cysteine;
X₇₂: (position 102 according to Kabat numbering) can be Tyr or any amino acid with a reactive group in its side chain, such as cysteine;
X₇₃: (position 10³ according to Kabat numbering) can be Trp or any amino acid with a reactive group in its side chain, such as cysteine;
X₇₄: (position 105 according to Kabat numbering) can be Arg or any amino acid with a reactive group in its side chain, such as cysteine;
X₇₅: (position 106 according to Kabat numbering) can be Gly or any amino acid with a reactive group in its side chain, such as cysteine;
X₇₆: (position 108 according to Kabat numbering) can be Gln, Leu, Arg, Pro, Glu, Lys, Ser, Thr, Met, Ala or His; preferably Gln or Leu, or any other amino acid with a reactive group in its side chain, such as cysteine;
X₇₇: (position 110 according to Kabat numbering) can be Thr or any amino acid with a reactive group in its side chain, such as cysteine;
X₇₈: (position 112 according to Kabat numbering) can be Ser or any amino acid with a reactive group in its side chain, such as cysteine;
X₇₉: (position 113 according to Kabat numbering) can be Ser or any amino acid with a reactive group in its side chain, such as cysteine;
X₈₀: is absent or Gly;
X₈₁: is absent or Gly;
X₈₂: is absent or Cys,
or a sequence which has 80% or more identity with SEQ ID NO.: 186, preferably a sequence which has 85% or more, 90% or more, 95% or more, 97% or more or 99% or more sequence identity with SEQ ID NO.: 186, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
27. The molecule of any of items 19 to 20 or 22-26, wherein the ISVD-derived building block comprises or, alternatively, consists of SEQ ID NO.: 206:

X₁ₐVQLVEX₁GGGZ₁VX₂AGGX₃LX₄IX₅CX₆AX₇X_{7b}GX_{7c}LSX₈YVLGWFRQ
APGX₉X₁₀REFVAAINWRGX₁₁ITIGPPX₁₂VEX₁₃RFX₁₄IX₁₅RX₁₆NX₁₇X₁₈
NTGYLQMNX₁₉LAPX_{19b}DTAZ₂YYCGAGTPLNPX₂₀AYIYX₂₁WSYDYWG
X₂₂GTZ₃VTVX₂₃SX₂₄X₂₅X₂₆

wherein
X₁ₐ (position 1 according to Kabat numbering) can be Glu or any amino acid with a reactive group in its side chain, such as cysteine;
X₁ (position 7 according to Kabat numbering) can be Ser or any amino acid with a reactive group in its side chain, such as cysteine;
Z₁ (position 11 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂ (position 13 according to Kabat numbering) can be Gln or any amino acid with a reactive group in its side chain, such as cysteine;
X₃ (position 17 according to Kabat numbering) can be Ser or any amino acid with a reactive group in its side chain, such as cysteine;
X₄ (position 19 according to Kabat numbering) can be Ser or any amino acid with a reactive group in its side chain, such as cysteine;
X₅: (position 21 according to Kabat numbering) can be Ser or any amino acid with a reactive group in its side chain, such as cysteine;
X₆: (position 23 according to Kabat numbering) can be Ala or any amino acid with a reactive group in its side chain, such as cysteine;
X₇: (position 25 according to Kabat numbering) can be Ser or any amino acid with a reactive group in its side chain, such as cysteine;
X_{7b}: (position 26 according to Kabat numbering) can be Gly or any amino acid with a reactive group in its side chain, such as cysteine;
X_{7c}: (position 28 according to Kabat numbering) can be Ser or any amino acid with a reactive group in its side chain, such as cysteine;
X₈: (position 31 according to Kabat numbering) can be Asn or any amino acid with a reactive group in its side chain, such as cysteine;
X₉: (position 43 according to Kabat numbering) can be Lys or any amino acid with a reactive group in its side chain, such as cysteine;
X₁₀: (position 44 according to Kabat numbering) can be Glu or any amino acid with a reactive group in its side chain, such as cysteine;
X₁₁: (position 55 according to Kabat numbering) can be Asp or any amino acid with a reactive group in its side chain, such as cysteine;
X₁₂: (position 62 according to Kabat numbering) can be Asn or any amino acid with a reactive group in its side chain, such as cysteine;
X₁₃: (position 65 according to Kabat numbering) can be Gly or any amino acid with a reactive group in its side chain, such as cysteine;
X₁₄: (position 68 according to Kabat numbering) can be Thr or any amino acid with a reactive group in its side chain, such as cysteine;
X₁₅: (position 70 according to Kabat numbering) can be Ser or any amino acid with a reactive group in its side chain, such as cysteine;
X₁₆: (position 72 according to Kabat numbering) can be Asp or any amino acid with a reactive group in its side chain, such as cysteine;
X₁₇: (position 74 according to Kabat numbering) can be Ala or any amino acid with a reactive group in its side chain, such as cysteine;
X₁₈: (position 75 according to Kabat numbering) can be Lys or any amino acid with a reactive group in its side chain, such as cysteine;
X₁₉: (position 82b according to Kabat numbering) can be Ser or any amino acid with a reactive group in its side chain, such as cysteine;
X_{19b}: (position 85 according to Kabat numbering) can be Asp or any amino acid with a reactive group in its side chain, such as cysteine;
Z₂: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂₀: (position100a according to Kabat numbering) can be Gly or any amino acid with a reactive group in its side chain, such as cysteine;
X₂₁: (position 100f according to Kabat numbering) can be Asp or any amino acid with a reactive group in its side chain, such as cysteine;
X₂₂: (position 105 according to Kabat numbering) can be Arg or any amino acid with a reactive group in its side chain, such as cysteine;
Z₃: (position 108 according to Kabat numbering) can be Gln, Leu, Arg, Pro, Glu, Lys, Ser, Thr, Met, Ala or His; preferably Gln or Leu;
X₂₃: (position 112 according to Kabat numbering) can be Ser or any amino acid with a reactive group in its side chain, such as cysteine;
X₂₄: is absent or Gly;
X₂₅: is absent or Gly;
X₂₆: is absent or Cys,
or a sequence which has 80% or more identity with SEQ ID NO.: 206, preferably a sequence which has 85% or more, 90% or more, 95% or more, 97% or more or 99% or more sequence identity with SEQ ID NO.: 206, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
28. The molecule of any of items 19 to 20 or 22 to 27, wherein the ISVD-derived building block comprises or, alternatively, consists of SEQ ID NO.: 185:

EVQLVEX₁GGGZ₁VX₂AGGX₃LX₄IX₅CX₆AX₇GGSLSX₈YVLGWFRQAPG
X₉X₁₀REFVAAINWRGX₁₁ITIGPPX₁₂VEX₁₃RFX₁₄IX₁₅RX₁₆NX₁₇X₁₈NTG
YLQMNX₁₉LAPDDTAZ₂YYCGAGTPLNPX₂₀AYIYX₂₁WSYDYWGX₂₂GTZ₃
VTVX₂₃SX₂₄X₂₅X₂₆

wherein
X₁ (position 7 according to Kabat numbering) can be Ser or any amino acid with a reactive group in its side chain, such as cysteine;
Z₁ (position 11 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂ (position 13 according to Kabat numbering) can be Gln or any amino acid with a reactive group in its side chain, such as cysteine;
X₃ (position 17 according to Kabat numbering) can be Ser or any amino acid with a reactive group in its side chain, such as cysteine;
X₄ (position 19 according to Kabat numbering) can be Ser or any amino acid with a reactive group in its side chain, such as cysteine;
X₅: (position 21 according to Kabat numbering) can be Ser or any amino acid with a reactive group in its side chain, such as cysteine;
X₆: (position 23 according to Kabat numbering) can be Ala or any amino acid with a reactive group in its side chain, such as cysteine;
X₇: (position 25 according to Kabat numbering) can be Ser or any amino acid with a reactive group in its side chain, such as cysteine;
X₈: (position 31 according to Kabat numbering) can be Asn or any amino acid with a reactive group in its side chain, such as cysteine;
X₉: (position 43 according to Kabat numbering) can be Lys or any amino acid with a reactive group in its side chain, such as cysteine;
X₁₀: (position 44 according to Kabat numbering) can be Glu or any amino acid with a reactive group in its side chain, such as cysteine;
X₁₁: (position 55 according to Kabat numbering) can be Asp or any amino acid with a reactive group in its side chain, such as cysteine;
X₁₂: (position 62 according to Kabat numbering) can be Asn or any amino acid with a reactive group in its side chain, such as cysteine;
X₁₃: (position 65 according to Kabat numbering) can be Gly or any amino acid with a reactive group in its side chain, such as cysteine;
X₁₄: (position 68 according to Kabat numbering) can be Thr or any amino acid with a reactive group in its side chain, such as cysteine;
X₁₅: (position 70 according to Kabat numbering) can be Ser or any amino acid with a reactive group in its side chain, such as cysteine;
X₁₆: (position 72 according to Kabat numbering) can be Asp or any amino acid with a reactive group in its side chain, such as cysteine;
X₁₇: (position 74 according to Kabat numbering) can be Ala or any amino acid with a reactive group in its side chain, such as cysteine;
X₁₈: (position 75 according to Kabat numbering) can be Lys or any amino acid with a reactive group in its side chain, such as cysteine;
X₁₉: (position 82b according to Kabat numbering) can be Ser or any amino acid with a reactive group in its side chain, such as cysteine;
Z₂: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂₀: (position100a according to Kabat numbering) can be Gly or any amino acid with a reactive group in its side chain, such as cysteine;
X₂₁: (position 100f according to Kabat numbering) can be Asp or any amino acid with a reactive group in its side chain, such as cysteine;
X₂₂: (position 105 according to Kabat numbering) can be Arg or any amino acid with a reactive group in its side chain, such as cysteine;
Z₃: (position 108 according to Kabat numbering) can be Gln, Leu, Arg, Pro, Glu, Lys, Ser, Thr, Met, Ala or His; preferably Gln or Leu;
X₂₃: (position 112 according to Kabat numbering) can be Ser or any amino acid with a reactive group in its side chain, such as cysteine;
X₂₄: is absent or Gly;
X₂₅: is absent or Gly;
X₂₆: is absent or Cys,
or a sequence which has 80% or more identity with SEQ ID NO.: 185, preferably a sequence which has 85% or more, 90% or more, 95% or more, 97% or more or 99% or more sequence identity with SEQ ID NO.: 185, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
29. The molecule of any of items 19 to 20 or 22 to 28, wherein the ISVD-derived building block comprises or, alternatively, consists of SEQ ID NO.: 185:

EVQLVEX₁GGGZ₁VX₂AGGX₃LX₄IX₅CX₆AX₇GGSLSX₈YVLGWFRQAPGX₉
X₁₀REFVAAINWRGX₁₁ITIGPPX₁₂VEX₁₃RFX₁₄IX₁₅RX₁₆NX₁₇X₁₈NTGY
LQMNX₁₉LAPDDTAZ₂YYCGAGTPLNPX₂₀AYIYX₂₁WSYDYWGX₂₂GTZ₃V
TVX₂₃SX₂₄X₂₅X₂₆

wherein
(a)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(b)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(c)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(d)
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(e)
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(f)
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent;
(g)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys,
or a sequence which has 80% or more identity with SEQ ID NO.: 185, preferably a sequence which has 85% or more, 90% or more, 95% or more, 97% or more or 99% or more sequence identity with SEQ ID NO.: 185, provided that the building block has a globular 3D structure, is soluble, has a size (molecular mass) of about 2.5 to about 70 kDa and does not specifically bind to any human protein.
30. The molecule of any of items 19 to 20 or 22 to 29, wherein the ISVD-derived building block comprises or, alternatively, consists of SEQ ID NO.: 185:

EVQLVEX₁GGGZ₁VX₂AGGX₃LX₄IX₅CX₆AX₇GGSLSX₈YVLGWFRQAPGX₉
X₁₀REFVAAINWRGX₁₁ITIGPPX₁₂VEX₁₃RFX₁₄IX₁₅RX₁₆NX₁₇X₁₈NTGYL
QMNX₁₉LAPDDTAZ₂YYCGAGTPLNPX₂₀AYIYX₂₁WSYDYWGX₂₂GTZ₃VTV
X₂₃SX₂₄X₂₅X₂₆

wherein
(a)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
(b)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
(c)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(d)
   X₁: (position 7 according to Kabat numbering) is Ser;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) can be any amino acid with a reactive group in its side chain, such as a cysteine, or a lysine, or a tyrosine, or a non-natural amino acid, preferably a cysteine;
   X₁₄: (position 68 according to Kabat numbering) is Thr;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(e)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
(f)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent, or
(g)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
(h)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) is Asp;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
(i)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) can be any amino acid with a reactive group in its side chain, such as a cysteine, or a lysine, or a tyrosine, or a non-natural amino acid, preferably a cysteine;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) is Thr;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys, or
(j)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) is Asn;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position100a according to Kabat numbering) is Gly;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is Gly;
   X₂₅: is Gly;
   X₂₆: is Cys,
or a sequence which has 80% or more identity with SEQ ID NO.: 185, preferably a sequence which has 85% or more, 90% or more, 95% or more, 97% or more or 99% or more sequence identity with SEQ ID NO.: 185, provided that the building block has a globular 3D structure, is soluble, has a size (molecular mass) of about 2.5 to about 70 kDa and does not specifically bind to any human protein.
31. The molecule of any of items 19 to 20 or 22 to 30, wherein the ISVD-derived building block comprises or, alternatively, consists of SEQ ID NO.: 185:

EVQLVEX₁GGGZ₁VX₂AGGX₃LX₄IX₅CX₆AX₇GGSLSX₈YVLGWFRQAPGX₉
X₁₀REFVAAINWRGX₁₁ITIGPPX₁₂VEX₁₃RFX₁₄IX₁₅RX₁₆NX₁₇X₁₈NTGYL
QMNX₁₉LAPDDTAZ₂YYCGAGTPLNPX₂₀AYIYX₂₁WSYDYWGX₂₂GTZ₃VTV
X₂₃SX₂₄X₂₅X₂₆

wherein
(a)
   X₁: (position 7 according to Kabat numbering) is Ser;
   Z₁: (position 11 according to Kabat numbering) is Leu or Val;
   X₂: (position 13 according to Kabat numbering) is Gln;
   X₃: (position 17 according to Kabat numbering) is Ser;
   X₄: (position 19 according to Kabat numbering) is Ser;
   X₅: (position 21 according to Kabat numbering) can be any amino acid with a reactive group in its side chain, such as a cysteine, or a lysine, or a tyrosine, or a non-natural amino acid, preferably a cysteine;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) can be any amino acid with a reactive group in its side chain, such as a cysteine, or a lysine, or a tyrosine, or a non-natural amino acid, preferably a cysteine;
   X₁₀: (position 44 according to Kabat numbering) is Glu;
   X₁₁: (position 55 according to Kabat numbering) is Asp;
   X₁₂: (position 62 according to Kabat numbering) is Asn;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) can be any amino acid with a reactive group in its side chain, such as a cysteine, or a lysine, or a tyrosine, or a non-natural amino acid, preferably a cysteine;
   X₁₇: (position 74 according to Kabat numbering) is Ala;
   X₁₈: (position 75 according to Kabat numbering) is Lys;
   X₁₉: (position 82b according to Kabat numbering) can be any amino acid with a reactive group in its side chain, such as a cysteine, or a lysine, or a tyrosine, or a non-natural amino acid, preferably a cysteine;
   Z₂: (position 89 according to Kabat numbering) is Val or Leu;
   X₂₀: (position 100a according to Kabat numbering) can be any amino acid with a reactive group in its side chain, such as a cysteine, or a lysine, or a tyrosine, or a non-natural amino acid, preferably a cysteine;
   X₂₁: (position 100f according to Kabat numbering) can be any amino acid with a reactive group in its side chain, such as a cysteine, or a lysine, or a tyrosine, or a non-natural amino acid, preferably a cysteine;
   X₂₂: (position 105 according to Kabat numbering) can be any amino acid with a reactive group in its side chain, such as a cysteine, or a lysine, or a tyrosine, or a non-natural amino acid, preferably a cysteine;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(b)
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) can be any amino acid with a reactive group in its side chain, such as a cysteine, or a lysine, or a tyrosine, or a non-natural amino acid, preferably a cysteine;
   X₃: (position 17 according to Kabat numbering) is Ser;
   X₄: (position 19 according to Kabat numbering) can be any amino acid with a reactive group in its side chain, such as a cysteine, or a lysine, or a tyrosine, or a non-natural amino acid, preferably a cysteine;
   X₅: (position 21 according to Kabat numbering) is Ser;
   X₆: (position 23 according to Kabat numbering) can be any amino acid with a reactive group in its side chain, such as a cysteine, or a lysine, or a tyrosine, or a non-natural amino acid, preferably a cysteine;
   X₇: (position 25 according to Kabat numbering) is Ser;
   X₈: (position 31 according to Kabat numbering) is Asn;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) is Thr;
   X₁₅: (position 70 according to Kabat numbering) can be any amino acid with a reactive group in its side chain, such as a cysteine, or a lysine, or a tyrosine, or a non-natural amino acid, preferably a cysteine;
   X₁₆: (position 72 according to Kabat numbering) is Asp;
   X₁₇: (position 74 according to Kabat numbering) can be any amino acid with a reactive group in its side chain, such as a cysteine, or a lysine, or a tyrosine, or a non-natural amino acid, preferably a cysteine;
   X₁₈: (position 75 according to Kabat numbering) is Lys;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) is Ser;
   X₂₄: is absent;
   X₂₅: is absent;
   X₂₆: is absent, or
(c)
   X₁: (position 7 according to Kabat numbering) can be any amino acid with a reactive group in its side chain, such as a cysteine, or a lysine, or a tyrosine, or a non-natural amino acid, preferably a cysteine;
   Z₁: (position 11 according to Kabat numbering) is Leu or Val;
   X₂: (position 13 according to Kabat numbering) is Gln;
   X₃: (position 17 according to Kabat numbering) can be any amino acid with a reactive group in its side chain, such as a cysteine, or a lysine, or a tyrosine, or a non-natural amino acid, preferably a cysteine;
   X₄: (position 19 according to Kabat numbering) is Ser;
   X₅: (position 21 according to Kabat numbering) is Ser;
   X₆: (position 23 according to Kabat numbering) is Ala;
   X₇: (position 25 according to Kabat numbering) is Ser;
   X₈: (position 31 according to Kabat numbering) can be any amino acid with a reactive group in its side chain, such as a cysteine, or a lysine, or a tyrosine, or a non-natural amino acid, preferably a cysteine;
   X₉: (position 43 according to Kabat numbering) is Lys;
   X₁₀: (position 44 according to Kabat numbering) can be any amino acid with a reactive group in its side chain, such as a cysteine, or a lysine, or a tyrosine, or a non-natural amino acid, preferably a cysteine;
   X₁₁: (position 55 according to Kabat numbering) can be any amino acid with a reactive group in its side chain, such as a cysteine, or a lysine, or a tyrosine, or a non-natural amino acid, preferably a cysteine;
   X₁₂: (position 62 according to Kabat numbering) can be any amino acid with a reactive group in its side chain, such as a cysteine, or a lysine, or a tyrosine, or a non-natural amino acid, preferably a cysteine;
   X₁₃: (position 65 according to Kabat numbering) is Gly;
   X₁₄: (position 68 according to Kabat numbering) can be any amino acid with a reactive group in its side chain, such as a cysteine, or a lysine, or a tyrosine, or a non-natural amino acid, preferably a cysteine;
   X₁₅: (position 70 according to Kabat numbering) is Ser;
   X₁₆: (position 72 according to Kabat numbering) is Asp;
   X₁₇: (position 74 according to Kabat numbering) is Ala;
   X₁₈: (position 75 according to Kabat numbering) can be any amino acid with a reactive group in its side chain, such as a cysteine, or a lysine, or a tyrosine, or a non-natural amino acid, preferably a cysteine;
   X₁₉: (position 82b according to Kabat numbering) is Ser;
   Z₂: (position 89 according to Kabat numbering) is Val or Leu;
   X₂₀: (position 100a according to Kabat numbering) is Gly;
   X₂₁: (position 100f according to Kabat numbering) is Asp;
   X₂₂: (position 105 according to Kabat numbering) is Arg;
   Z₃: (position 108 according to Kabat numbering) is Gln or Leu;
   X₂₃: (position 112 according to Kabat numbering) can be any amino acid with a reactive group in its side chain, such as a cysteine, or a lysine, or a tyrosine, or a non-natural amino acid, preferably a cysteine;
   X₂₄: is absent;
   X₂₅: is absent;
   X₂₆: is absent.
or a sequence which has 80% or more identity with SEQ ID NO.: 185, preferably a sequence which has 85% or more, 90% or more, 95% or more, 97% or more or 99% or more sequence identity with SEQ ID NO.: 185, provided that the building block has a globular 3D structure, is soluble, has a size (molecular mass) of about 2.5 to about 70 kDa and does not specifically bind to any human protein.
32. The molecule of any of items 19 to 20 or 22 to 31, wherein the ISVD-derived building block additionally comprises an extra cysteine and/or an extra tyrosine at one or both ends of the polypeptide defined by SEQ ID NO.: 185, 206 or 186, preferably wherein the cysteine is preceded/followed by a flexible tag (sequence), such as a (GG) tag, and preferably wherein the tyrosine is preceded/followed by flexible tags, such as (GG) or (G₄S₁)₁₋₃GG tags.
33. The molecule of any of items 19 to 20 or 22 to 32, wherein the ISVD-derived building block comprises or, alternatively consists of, one of the polypeptides as defined in SEQ ID NOs.: 80-95, 175 or 222-224, or a sequence which has 80% or more identity with SEQ ID NOs.: 80-95, 175 or 222-224, preferably a sequence which has 85% or more, 90% or more, 95% or more, 97% or more or 99% or more sequence identity with SEQ ID NOs.: 80-95, 175 or 222-224.
34. The molecule of any of items 19 or 20, wherein the protein-based building block is derived from a DARPin protein.
35. The molecule of any of items 19, 20 or 34, wherein the at least one protein-based building block is derived from the polypeptide as defined in SEQ ID NO.: 187.
36. The molecule of any of items 19, 20 or 34 to 35, wherein the at least one protein-based building block comprises, or alternatively, consists of, SEQ ID NO.: 188:

X₁X₂GX₃X₄LLX₅AAX₆X₇X₈X₉X₁₀X₁₁X₁₂VX₁₃X₁₄LMX₁₅X₁₆X₁₇AX₁₈VX₁₉A
X₂₀X₂₁X₂₂X₂₃GX₂₄TPLHLAAX₂₅X₂₆X₂₇X₂₈X₂₉X₃₀IVX₃₁VLLX₃₂X₃₃X₃₄A
X₃₅VX₃₆AX₃₇DX₃₈X₃₉GATPLHLAAX₄₀X₄₁X₄₂X₄₃X₄₄X₄₅IVX₄₆VLLX₄₇X₄₈
X₄₉AX₅₀VX₅₁AX₅₂DX₅₃X₅₄GATPLHX₅₅AAX₅₆X₅₇X₅₈X₅₉X₆₀X₆₁IVX₆₂X₆₃
LX₆₄X₆₅X₆₆X₆₇AX₆₈X₆₉X₇₀AX₇₁DX₇₂X₇₃X₇₄X₇₅TAX₇₆X₇₇ISX₇₈X₇₉X₈₀X₈₁
X₈₂X₈₃X₈₄LAX₈₅X₈₆LX₈₇X₈₈X₈₉X₉₀,

wherein
X₁ can be Asp or any amino acid with a reactive group in its side chain, such as cysteine;
X₂ can be Leu or any amino acid with a reactive group in its side chain, such as cysteine;
X₃ can be Lys or any amino acid with a reactive group in its side chain, such as cysteine;
X₄ can be Lys or any amino acid with a reactive group in its side chain, such as cysteine;
X₅ can be Glu or any amino acid with a reactive group in its side chain, such as cysteine;
X₆ can be Arg or any amino acid with a reactive group in its side chain, such as cysteine;
X₇ can be Ala or any amino acid with a reactive group in its side chain, such as cysteine;
X₈ can be Gly or any amino acid with a reactive group in its side chain, such as cysteine;
X₉ can be Gln or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Arg or any amino acid with a reactive group in its side chain, such as cysteine;
X₁₄ can be Ile or any amino acid with a reactive group in its side chain, such as cysteine;
X₁₅ can be Ala or any amino acid with a reactive group in its side chain, such as cysteine;
X₁₆ can be Asn or any amino acid with a reactive group in its side chain, such as cysteine;
X₁₇ can be Gly or any amino acid with a reactive group in its side chain, such as cysteine;
X₁₈ can be Asp or any amino acid with a reactive group in its side chain, such as cysteine;
X₁₉ can be Asn or any amino acid with a reactive group in its side chain, such as cysteine;
X₂₀ can be His or any amino acid with a reactive group in its side chain, such as cysteine;
X₂₁ can be Asp or any amino acid with a reactive group in its side chain, such as cysteine;
X₂₂ can be Thr or any amino acid with a reactive group in its side chain, such as cysteine;
X₂₃ can be Phe or any amino acid with a reactive group in its side chain, such as cysteine;
X₂₄ can be Phe or any amino acid with a reactive group in its side chain, such as cysteine;
X₂₅ can be Leu or any amino acid with a reactive group in its side chain, such as cysteine;
X₂₆ can be Tyr or any amino acid with a reactive group in its side chain, such as cysteine;
X₂₇ can be Gly or any amino acid with a reactive group in its side chain, such as cysteine;
X₂₈ can be His or any amino acid with a reactive group in its side chain, such as cysteine
X₂₉ can be Leu or any amino acid with a reactive group in its side chain, such as cysteine
X₃₀ can be Glu or any amino acid with a reactive group in its side chain, such as cysteine
X₃₁ can be Glu or any amino acid with a reactive group in its side chain, such as cysteine
X₃₂ can be Lys or any amino acid with a reactive group in its side chain, such as cysteine
X₃₃ can be Asn or any amino acid with a reactive group in its side chain, such as cysteine
X₃₄ can be Gly or any amino acid with a reactive group in its side chain, such as cysteine
X₃₅ can be Asp or any amino acid with a reactive group in its side chain, such as cysteine
X₃₆ can be Asn or any amino acid with a reactive group in its side chain, such as cysteine
X₃₇ can be Asp or any amino acid with a reactive group in its side chain, such as cysteine
X₃₈ can be Ser or any amino acid with a reactive group in its side chain, such as cysteine
X₃₉ can be Tyr or any amino acid with a reactive group in its side chain, such as cysteine;
X₄₀ can be Met or any amino acid with a reactive group in its side chain, such as cysteine;
X₄₁ can be Arg or any amino acid with a reactive group in its side chain, such as cysteine;
X₄₂ can be Gly or any amino acid with a reactive group in its side chain, such as cysteine;
X₄₃ can be His or any amino acid with a reactive group in its side chain, such as cysteine;
X₄₄ can be Leu or any amino acid with a reactive group in its side chain, such as cysteine;
X₄₅ can be Glu or any amino acid with a reactive group in its side chain, such as cysteine;
X₄₆ can be Glu or any amino acid with a reactive group in its side chain, such as cysteine;
X₄₇ can be Lys or any amino acid with a reactive group in its side chain, such as cysteine;
X₄₈ can be Tyr or any amino acid with a reactive group in its side chain, such as cysteine;
X₄₉ can be Gly or any amino acid with a reactive group in its side chain, such as cysteine;
X₅₀ can be Asp or any amino acid with a reactive group in its side chain, such as cysteine;
X₅₁ can be Asn or any amino acid with a reactive group in its side chain, such as cysteine;
X₅₂ can be Ala or any amino acid with a reactive group in its side chain, such as cysteine;
X₅₃ can be Glu or any amino acid with a reactive group in its side chain, such as cysteine;
X₅₄ can be Glu or any amino acid with a reactive group in its side chain, such as cysteine;
X₅₅ can be Leu or any amino acid with a reactive group in its side chain, such as cysteine;
X₅₆ can be Lys or any amino acid with a reactive group in its side chain, such as cysteine;
X₅₇ can be Ala or any amino acid with a reactive group in its side chain, such as cysteine;
X₅₈ can be Gly or any amino acid with a reactive group in its side chain, such as cysteine;
X₅₉ can be His or any amino acid with a reactive group in its side chain, such as cysteine;
X₆₀ can be Leu or any amino acid with a reactive group in its side chain, such as cysteine;
X₆₁ can be Glu or any amino acid with a reactive group in its side chain, such as cysteine;
X₆₂ can be Glu or any amino acid with a reactive group in its side chain, such as cysteine;
X₆₃ can be Val or any amino acid with a reactive group in its side chain, such as cysteine;
X₆₄ can be Leu or any amino acid with a reactive group in its side chain, such as cysteine;
X₆₅ can be Lys or any amino acid with a reactive group in its side chain, such as cysteine;
X₆₆ can be Asn or any amino acid with a reactive group in its side chain, such as cysteine;
X₆₇ can be Gly or any amino acid with a reactive group in its side chain, such as cysteine;
X₆₈ can be Asp or any amino acid with a reactive group in its side chain, such as cysteine;
X₆₉ can be Val or any amino acid with a reactive group in its side chain, such as cysteine;
X₇₀ can be Asn or any amino acid with a reactive group in its side chain, such as cysteine;
X₇₁ can be Gln or any amino acid with a reactive group in its side chain, such as cysteine;
X₇₂ can be Lys or any amino acid with a reactive group in its side chain, such as cysteine;
X₇₃ can be Phe or any amino acid with a reactive group in its side chain, such as cysteine;
X₇₄ can be Gly or any amino acid with a reactive group in its side chain, such as cysteine;
X₇₅ can be Lys or any amino acid with a reactive group in its side chain, such as cysteine;
X₇₆ can be Phe or any amino acid with a reactive group in its side chain, such as cysteine;
X₇₇ can be Asp or any amino acid with a reactive group in its side chain, such as cysteine;
X₇₈ can be Ile or any amino acid with a reactive group in its side chain, such as cysteine;
X₇₉ can be Asp or any amino acid with a reactive group in its side chain, such as cysteine;
X₈₀ can be Asn or any amino acid with a reactive group in its side chain, such as cysteine;
X₈₁ can be Gly or any amino acid with a reactive group in its side chain, such as cysteine;
X₈₂ can be Asn or any amino acid with a reactive group in its side chain, such as cysteine;
X₈₃ can be Glu or any amino acid with a reactive group in its side chain, such as cysteine;
X₈₄ can be Asp or any amino acid with a reactive group in its side chain, such as cysteine;
X₈₅ can be Glu or any amino acid with a reactive group in its side chain, such as cysteine;
X₈₆ can be Ile or any amino acid with a reactive group in its side chain, such as cysteine;
X₈₇ can be Gln or any amino acid with a reactive group in its side chain, such as cysteine;
X₈₈ can be Lys or any amino acid with a reactive group in its side chain, such as cysteine;
X₈₉ can be absent or Leu;
X₉₀ can be absent or Cys
or a sequence which has 80% or more identity with SEQ ID NO.: 188, preferably a sequence which has 85% or more, 90% or more, 95% or more, 97% or more or 99% or more sequence identity with SEQ ID NO.: 188, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
37. The molecule of any of items 19, 20 or 34 to 36, wherein the at least one protein-based building block comprises, or alternatively, consists of, SEQ ID NO.: 189,

DLGKX₁LLEAARAGQDDEVRILMANGADVNAHDTFGFTPLHLAALYGHL
X₂IVEVLLKNGAX₃VNAX₄DSYGATPLHLAAMRGHLX₅IVX₆VLLKYGAX₇
VX₈AX₉DEX₁₀GATPLHLAAKAGHLX₁₁IVEVLLKNGAX₁₂VNAQDKFGKT
AFDISIX₁₃NGNEX₁₄LAEILQX₁₅X₁₆X₁₇,

wherein
X₁ can be Lys or any amino acid with a reactive group in its side chain, such as cysteine;
X₂ can be Glu or any amino acid with a reactive group in its side chain, such as cysteine;
X₃ can be Asp or any amino acid with a reactive group in its side chain, such as cysteine;
X₄ can be Asp or any amino acid with a reactive group in its side chain, such as cysteine;
X₅ can be Glu or any amino acid with a reactive group in its side chain, such as cysteine;
X₆ can be Glu or any amino acid with a reactive group in its side chain, such as cysteine;
X₇ can be Asp or any amino acid with a reactive group in its side chain, such as cysteine;
X₈ can be Asn or any amino acid with a reactive group in its side chain, such as cysteine;
X₉ can be Ala or any amino acid with a reactive group in its side chain, such as cysteine;
X₁₀ can be Glu or any amino acid with a reactive group in its side chain, such as cysteine;
X₁₁ can be Glu or any amino acid with a reactive group in its side chain, such as cysteine;
X₁₂ can be Asp or any amino acid with a reactive group in its side chain, such as cysteine;
X₁₃ can be Asp or any amino acid with a reactive group in its side chain, such as cysteine;
X₁₄ can be Asp or any amino acid with a reactive group in its side chain, such as cysteine;
X₁₅ can be Lys or any amino acid with a reactive group in its side chain, such as cysteine;
X₁₆ can be absent or Leu;
X₁₇ can be absent or Cys,
or a sequence which has 80% or more identity with SEQ ID NO.: 189, preferably a sequence which has 85% or more, 90% or more, 95% or more, 97% or more or 99% or more sequence identity with SEQ ID NO.: 189, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
38. The molecule of any of items 19, 20 or 34 to 37, wherein the at least one protein-based building block comprises, or alternatively, consists of, SEQ ID NO.: 181:

DLGKKLLEAARAGQDDEVRILMANGADVNAHDTFGFTPLHLAALYGHLE
IVEVLLKNGADVNADDSYGATPLHLAAMRGHLEIVX₁VLLKYGADVX₂A
ADEEGATPLHLAAKAGHLEIVEVLLKNGADVNAQDKFGKTAFDISIX₃N
GNEX₄LAEILQKX₅X₆,

wherein
X₁ can be Glu or any amino acid with a reactive group in its side chain, such as cysteine;
X₂ can be Asn or any amino acid with a reactive group in its side chain, such as cysteine;
X₃ can be Asp or any amino acid with a reactive group in its side chain, such as cysteine;
X₄ can be Asp or any amino acid with a reactive group in its side chain, such as cysteine;
X₅ can be absent or Leu; and
X₆ can be absent or Cys.
or a sequence which has 80% or more identity with SEQ ID NO.: 181, preferably a sequence which has 85% or more, 90% or more, 95% or more, 97% or more or 99% or more sequence identity with SEQ ID NO.: 181, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
39. The molecule of any of items 19, 20 or 34 to 38, wherein the DARPin-derived building block additionally comprises an extra cysteine and/or an extra tyrosine at one or both ends of the polypeptide defined by SEQ ID NOs.: 181 or 188-189, preferably wherein the cysteine is preceded/followed by a flexible tag (sequence), such as a (GG) tag, and preferably wherein the tyrosine is preceded/followed by flexible tags, such as (GG) or (G₄S₁)₁₋₃GG tags.
40. The molecule of item 39, wherein the DARPin-derived building block comprises or, alternatively consists of, SEQ ID NO.: 182, or a sequence which has 80% or more identity with SEQ ID NO.: 182, preferably a sequence which has 85% or more, 90% or more, 95% or more, 97% or more or 99% or more sequence identity with SEQ ID NO.: 181, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
41. The molecule of any of items 19, 20 or 34 to 39, wherein the DARPin-derived building block comprises or, alternatively consists of, one of the polypeptides as defined in SEQ ID NOs.: 96-98, 199 or 208, or a sequence which has 80% or more identity with SEQ ID NOs.: 96-98, 199 or 208, preferably a sequence which has 85% or more, 90% or more, 95% or more, 97% or more or 99% or more sequence identity with SEQ ID NOs.: 96-98, 199 or 208.
42. The molecule of any of items 19 or 20, wherein the at least one protein-based building block is an affibody-derived building block or an affitin-derived building block.
43. The molecule of any of items 19 or 21, wherein the at least one protein-based building block is derived from cyclin-dependent kinase subunit 1 (CKS1) protein.
44. The molecule of any of items 19, 21 or 43, wherein the at least one protein-based building block is derived from the polypeptide as defined in SEQ ID NO.: 190.
45. The molecule of any of items 19, 21 or 43 to 44, wherein the at least one protein-based building block comprises, or alternatively, consists of, SEQ ID NO.: 191:

X₁X₂X₃X₄IX₅X₆SX₇X₈X₉X₁₀X₁₁X₁₂X₁₃X₁₄X₁₅X₁₆X₁₇X₁₈VX₁₉LPX₂₀X₂₁X₂₂
AX₂₃X₂₄VX₂₅X_{23b}X_{24b}X_{25b}X₂₆MX₂₇X₂₈X₂₉X₃₀WX₃₁X₃₂LX₃₃VX₃₄QX₃₅X₃₆X₃₇
WX₃₈HX₃₉X₄₀X₄₁X₄₂X₄₃X₄₄X₄₅X₄₆X₄₇ILLFX₄₈X₄₉X₅₀X₅₁X₅₂X₅₃X₅₄X₅₅X₅₆
X₅₇,

wherein
X₁ can be Ser or any amino acid with a reactive group in its side chain, such as cysteine;
X₂ can be His or any amino acid with a reactive group in its side chain, such as cysteine;
X₃ can be Lys or any amino acid with a reactive group in its side chain, such as cysteine;
X₄ can be Gln or any amino acid with a reactive group in its side chain, such as cysteine;
X₅ can be Tyr or any amino acid with a reactive group in its side chain, such as cysteine;
X₆ can be Tyr or any amino acid with a reactive group in its side chain, such as cysteine;
X₇ can be Asp or any amino acid with a reactive group in its side chain, such as cysteine;
X₈ can be Lys or any amino acid with a reactive group in its side chain, such as cysteine;
X₉ can be Tyr or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Glu or any amino acid with a reactive group in its side chain, such as cysteine;
X₁₄ can be Phe or any amino acid with a reactive group in its side chain, such as cysteine;
X₁₅ can be Glu or any amino acid with a reactive group in its side chain, such as cysteine;
X₁₆ can be Tyr or any amino acid with a reactive group in its side chain, such as cysteine;
X₁₇ can be Arg or any amino acid with a reactive group in its side chain, such as cysteine;
X₁₈ can be His or any amino acid with a reactive group in its side chain, such as cysteine;
X₁₉ can be Met or any amino acid with a reactive group in its side chain, such as cysteine;
X₂₀ can be Lys or any amino acid with a reactive group in its side chain, such as cysteine;
X₂₁ can be Asp or any amino acid with a reactive group in its side chain, such as cysteine;
X₂₂ can be Ile or any amino acid with a reactive group in its side chain, such as cysteine;
X₂₃ can be Lys or any amino acid with a reactive group in its side chain, such as cysteine;
X₂₄ can be Leu or any amino acid with a reactive group in its side chain, such as cysteine;
X₂₅ can be Pro or any amino acid with a reactive group in its side chain, such as cysteine;
X₂₃b can be Lys or any amino acid with a reactive group in its side chain, such as cysteine;
X₂₄b can be Thr or any amino acid with a reactive group in its side chain, such as cysteine;
X₂₅b can be His or any amino acid with a reactive group in its side chain, such as cysteine;
X₂₆ can be Leu or any amino acid with a reactive group in its side chain, such as cysteine;
X₂₇ can be Ser or any amino acid with a reactive group in its side chain, such as cysteine;
X₂₈ can be Glu or any amino acid with a reactive group in its side chain, such as cysteine;
X₂₉ can be Ser or any amino acid with a reactive group in its side chain, such as cysteine;
X₃₀ can be Glu or any amino acid with a reactive group in its side chain, such as cysteine;
X₃₁ can be Arg or any amino acid with a reactive group in its side chain, such as cysteine;
X₃₂ can be Asn or any amino acid with a reactive group in its side chain, such as cysteine;
X₃₃ can be Gly or any amino acid with a reactive group in its side chain, such as cysteine;
X₃₄ can be Gln or any amino acid with a reactive group in its side chain, such as cysteine;
X₃₅ can be Ser or any amino acid with a reactive group in its side chain, such as cysteine;
X₃₆ can be Gln or any amino acid with a reactive group in its side chain, such as cysteine;
X₃₇ can be Gly or any amino acid with a reactive group in its side chain, such as cysteine;
X₃₈ can be Val or any amino acid with a reactive group in its side chain, such as cysteine;
X₃₉ can be Tyr or any amino acid with a reactive group in its side chain, such as cysteine;
X₄₀ can be Met or any amino acid with a reactive group in its side chain, such as cysteine;
X₄₁ can be Ile or any amino acid with a reactive group in its side chain, such as cysteine;
X₄₂ can be His or any amino acid with a reactive group in its side chain, such as cysteine;
X₄₃ can be Glu or any amino acid with a reactive group in its side chain, such as cysteine;
X₄₄ can be Pro or any amino acid with a reactive group in its side chain, such as cysteine;
X₄₅ can be Glu or any amino acid with a reactive group in its side chain, such as cysteine;
X₄₆ can be Pro or any amino acid with a reactive group in its side chain, such as cysteine;
X₄₇ can be His or any amino acid with a reactive group in its side chain, such as cysteine;
X₄₈ can be Arg or any amino acid with a reactive group in its side chain, such as cysteine;
X₄₉ can be Arg or any amino acid with a reactive group in its side chain, such as cysteine;
X₅₀ can be Pro or any amino acid with a reactive group in its side chain, such as cysteine;
X₅₁ can be Leu or any amino acid with a reactive group in its side chain, such as cysteine;
X₅₂ can be Pro or any amino acid with a reactive group in its side chain, such as cysteine;
X₅₃ can be Lys or any amino acid with a reactive group in its side chain, such as cysteine;
X₅₄ can be Lys or any amino acid with a reactive group in its side chain, such as cysteine;
X₅₅ can be Pro or any amino acid with a reactive group in its side chain, such as cysteine;
X₅₆ can be Lys or any amino acid with a reactive group in its side chain, such as cysteine;
X₅₇ can be Lys or any amino acid with a reactive group in its side chain, such as cysteine,
or a sequence which has 80% or more identity with SEQ ID NO.: 191, preferably a sequence which has 85% or more, 90% or more, 95% or more, 97% or more or 99% or more sequence identity with SEQ ID NO.: 191, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.
46. The molecule of any of items 19, 21 or 43 to 45, wherein the at least one protein-based building block comprises, or alternatively, consists of, SEQ ID NO.: 205:

SHKQIYYSX₁X₂X₃X₄X₅EEFEYRHVX₆LPKDIAKLVPX₇THLMSESEWRNL
GVQQSX₈GWVHYX₉IHEPEPHILLFRRPLPKKPKX₁₀,

wherein
X₁ can be Asp or any amino acid with a reactive group in its side chain, such as cysteine;
X₂ can be Lys or any amino acid with a reactive group in its side chain, such as cysteine;
X₃ can be Tyr or any amino acid with a reactive group in its side chain, such as cysteine;
X₄ can be Asp or any amino acid with a reactive group in its side chain, such as cysteine;
X₅ can be Asp or any amino acid with a reactive group in its side chain, such as cysteine;
X₆ can be Met or any amino acid with a reactive group in its side chain, such as cysteine;
X₇ can be Lys or any amino acid with a reactive group in its side chain, such as cysteine;
X₈ can be Gln or any amino acid with a reactive group in its side chain, such as cysteine;
X₉ can be Met or any amino acid with a reactive group in its side chain, such as cysteine;
X₁₀ can be Lys or any amino acid with a reactive group in its side chain, such as cysteine,
or a sequence which has 80% or more identity with SEQ ID NO.: 205, preferably a sequence which has 85% or more, 90% or more, 95% or more, 97% or more or 99% or more sequence identity with SEQ ID NO.: 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein
47. The molecule of any of items 19, 21 or 43 to 46, wherein the at least one protein-based building block comprises, or alternatively, consists of, SEQ ID NO.: 192:

X₁HKX₂IYYSDX₃YX₄DEEFEYRHVMLPX₅DIAX₆LVPX₇THLMSESEWRNL
GVQQSQGWVHYMIHEPEPHILLFRRPLPKKPKK

wherein
X₁ can be Ser or any amino acid with a reactive group in its side chain, such as cysteine;
X₂ can be Gln or any amino acid with a reactive group in its side chain, such as cysteine;
X₃ can be Lys or any amino acid with a reactive group in its side chain, such as cysteine;
X₄ can be Asp or any amino acid with a reactive group in its side chain, such as cysteine;
X₅ can be Lys or any amino acid with a reactive group in its side chain, such as cysteine;
X₆ can be Lys or any amino acid with a reactive group in its side chain, such as cysteine;
X₇ can be Lys or any amino acid with a reactive group in its side chain, such as cysteine,
or a sequence which has 80% or more identity with SEQ ID NO.: 192, preferably a sequence which has 85% or more, 90% or more, 95% or more, 97% or more or 99% or more sequence identity with SEQ ID NO.: 192, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein
48. The molecule of any of items 19, 21 or 43 to 47, wherein the small globular human protein-derived building block additionally comprises an extra cysteine and/or an extra tyrosine at one or both ends of the polypeptide defined by SEQ ID NOs.: 191-192 or 205, preferably wherein the cysteine is preceded/followed by a flexible tag (sequence), such as a (GG) tag, and preferably wherein the tyrosine is preceded/followed by flexible tags, such as (GG) or (G₄S₁)₁₋₃GG tags.
49. The molecule of any of items 19, 21 or 43 to 48, wherein the small globular human protein-derived building block comprises or, alternatively consists of, one of the polypeptides as defined in SEQ ID NOs.: 99-105, or a sequence which has 80% or more identity with SEQ ID NOs.: 99-105, preferably a sequence which has 85% or more, 90% or more, 95% or more, 97% or more or 99% or more sequence identity with SEQ ID NOs.: 99-105.
50. The molecule of any of items 1 to 49, wherein the molecule comprises at least one protein-based building block and at least one further moiety or cargo, preferably wherein the at least one further moiety or cargo is selected from
   a) a half-life extending (HLE) moiety, such as PEG, and/or
   b) a targeting moiety, such as an EGFR-targeting moiety, e.g., GE11 peptide; or an anti-EGFR V_{HH} and/or
   c) a therapeutic moiety or precursor therefrom, such as a Death receptor 5 (DR5) antagonist;
   d) an imaging moiety, such as deferoxamine (DFO)
   e) a toxic moiety, such as DM4 or cryptophycin;
   f) siRNA;
   g) vitamins, such as folate;
   h) Toll-like receptor agonists, such as resiquimod;
   i) glycans, such as bis mannose 6 phosphate (bisM6P) or mannose 6 phosphate (M6P); and/or
   j) lipids, such as short-chain fatty acids.
51. The molecule of item 50, wherein the molecule comprises at least one protein-based carrier building block and at least one cargo attached to one of the conjugation sites or attachment points present in the protein-based building block, preferably to a conjugation site or attachment point which is the side chain of an amino acid preferably located at a solvent-accessible position of the protein-based building block.
52. The molecule of item 51, wherein the at least one protein-based carrier building block is an ISVD-derived building block, preferably derived from a V_{H}, a humanized V_{H}, a human V_{H}, a V_{HH}, a humanized V_{HH} or a camelized V_{H} (derived from a heavy-chain ISVD), more preferably derived from an ISVD belonging to the “V_{H}3 class”.
53. The molecule of item 52, wherein the at least one cargo attached to the at least one building block is an ISVD.
54. The molecule of any one of items 50 to 53, wherein the at least one cargo is directly attached to the at least one protein-based building block, or wherein the at least one cargo is attached to the at least one protein-based building block through a linker.
55. The molecule of item 54, wherein the linker is a cleavable linker.
56. The molecule of any of items 54 or 55, wherein the linker is a peptide linker.
57. The molecule of any of items 54 or 55, wherein the linker is not a peptide linker.
58. The molecule of any of items 54 to 56, wherein the linker is an amino acid or an amino acid sequence, preferably of between 1 and 50, such as for example Gly-Ser linkers ((glyₓser_{y})_{z}), or A3, GS30, GS15, GS9 and GS7 linkers, or a linker as defined in SEQ ID NOs.: 158-169 or 193-196, more preferably a linker as defined in SEQ ID NO.: 163.
59. The molecule of any of items 54 to 55 or 57, wherein the linker is a linear or branched polyethylene glycol (PEG) moiety, preferably with a molecular weight of about 1-60 kDa, preferably with a weight of about 1-10 kDa, such as 5 kDa or 10 kDa.
60. The molecule of any of items 54 to 56 or 58, wherein the linker is an ELNN polypeptide.
61. The molecule of item 54, wherein the linker is an APN-maleimide linker (3-(4-(2,5-dioxo-2,5-dihydro-1H-pyrrol-1-yl)phenyl)propiolonitrile, MAPN) or a bis-maleimido-PEG3 (BM(PEG)3) linker (BM(PEG)3 (1,11-bismaleimido-triethyleneglycol)).
62. The molecule of any of items 50 to 61, wherein the cargo is an (in vivo) half-life extending moiety.
63. The molecule of item 62, wherein the cargo is a PEG molecule, an ELNN polypeptide or an albumin-binding polypeptide, preferably a PEG molecule, more preferably a PEG molecule with a MW of less than 20 kDa, such as less than 10 kDa, or less than 5 kDa, even more preferably a 1-5 kDa PEG molecule,
64. The molecule of any of items 62 to 63, wherein the cargo is an albumin-binding ISVD.
65. The molecule of any of items 62 to 64, wherein the cargo comprises or, alternatively consists of, a polypeptide as defined in any one of SEQ ID NOs.: 50-64 or 106, preferably SEQ ID NOs.: 63 or 106.
66. The molecule of any of items 1 to 65, wherein the molecule comprises or, alternatively consists of, a polypeptide as defined in any one of SEQ ID NOs.: 107-127, 170-174, 176 or 200.
67. A nucleic acid encoding the molecule as defined in any one of items 1 to 66, part of the molecule as defined in any one of items 1 to 66 and/or the protein-based building block as defined in any one of items 1 to 49.
68. A vector comprising the nucleic acid as defined in item 67.
69. A composition comprising the molecule as defined in any one of items 1 to 66, such as a pharmaceutical composition.
70. A method for producing the molecule as defined in any one of items 1 to 66, wherein the method comprises:
   a) expressing, in a suitable host cell or host organism or in another suitable expression system, a nucleic acid sequence encoding the at least one protein-based carrier building block and/or the molecule or part of the molecule as defined in any one of items 1-66;
   b) optionally isolating and/or purifying the at least one protein-based carrier building block and/or the molecule or part of the molecule expressed in a);
   c) optionally conjugating one or more (further) cargos to the attachment point(s) or conjugation sites(s) of the protein-based carrier building block.
71. A method for producing the molecule as defined in any one of items 1 to 66, wherein the method comprises:
   a) chemically synthesizing the at least one protein-based carrier building block and/or the molecule or part of the molecule as defined in any one of items 1 to 66, preferably by using solid-phase peptide synthesis;
   b) optionally isolating and/or purifying the at least one protein-based carrier building block and/or the molecule or part of the molecule synthesized in a);
   c) optionally conjugating one or more (further) cargos to the attachment point(s) or conjugation sites(s) of the protein-based carrier building block.
72. The molecule according to any one of items 1 to 66 or the composition according to item 69 for use in medicine.
73. The molecule according to any one of items 1 to 66 or the composition according to item 69 for use in the prophylactic and/or therapeutic treatment of an autoimmune/inflammatory disease, an infectious disease and/or cancer, such as hematological (blood) and solid tumor cancer disease.
74. The molecule according to any one of items 1 to 66 or the composition according to item 69 for use as a vaccine.
75. A vaccine comprising a molecule as defined in any one of items 1 to 66 or the composition as defined in item 69, optionally further comprising an adjuvant.
76. The molecule of item 50 to 61, wherein the molecule comprises at least one ISVD-derived building block and at least one cargo molecule, wherein the cargo molecule is a therapeutic moiety.
77. The molecule of item 76, wherein the therapeutic moiety is a Death receptor 5 (DR5) antagonist, such as one or more anti-DR5 ISVDs, e.g. such as three anti-DR5 ISVDs.
78. The molecule of item 76, wherein the therapeutic moiety is an EGFR antagonist, such as one or more anti-EGFR ISVDs.
79. The molecule of item 50 to 61, wherein the molecule comprises at least one ISVD-derived building block and at least one cargo molecule, wherein the cargo molecule is a toxic moiety.
80. The molecule of item 79, wherein the toxic moiety is resiquimod.
81. The molecule of item 79, wherein the toxic moiety is cryptophycin.
82. The molecule of item 79, wherein the toxic moiety is DM4.
83. The molecule of item 50 to 61, wherein the molecule comprises at least one ISVD-derived building block and at least one cargo molecule, wherein the cargo molecule is a targeting moiety.
84. The molecule of item 83, wherein the targeting moiety is an EGFR targeting moiety, such as GE1 peptide or an anti-EGFR VHH.
85. The molecule of item 50 to 61, wherein the molecule comprises at least one ISVD-derived building block and at least one cargo molecule, wherein the cargo molecule is a nucleic acid.
86. The molecule of item 85, wherein the nucleic acid is siRNA.
87. The molecule of item 50 to 61, wherein the molecule comprises at least one ISVD-derived building block and at least one cargo molecule, wherein the cargo molecule is a vitamin.
88. The molecule of item 87, wherein the vitamin is folate.
89. The molecule of item 50 to 61, wherein the molecule comprises at least one ISVD-derived building block and at least one cargo molecule, wherein the cargo molecule is a glycan.
90. The molecule of item 89, wherein the glycan is mannose-6-phosphate or bis-mannose-6-phosphate.
91. The molecule of item 50 to 61, wherein the molecule comprises at least one ISVD-derived building block and at least one cargo molecule, wherein the cargo molecule is a lipid.
92. The molecule of item 91, wherein the lipid is a short-chain fatty acid.
93. The molecule of item 50 to 61, wherein the molecule comprises at least one ISVD-derived building block and at least two different cargo molecules.
94. The molecule of item 93, wherein the two different cargo molecules are a half-life extending moiety, such as PEG, and a radiolabel, such as ⁸⁹Zr-DFO.
95. The molecule of item 93, wherein the two different cargo molecules are a half-life extending moiety, such as PEG, and a toxic moiety, such as cryptophycin, DM4 or resiquimod.
96. The molecule of item 50 to 61, wherein the molecule comprises at least one DARPin-derived building block and at least one cargo molecule, wherein the cargo molecule is a targeting moiety.
97. The molecule of item 96, wherein the targeting moiety is an EGFR targeting moiety, such as GE1 peptide or an anti-EGFR VHH.
98. The molecule of item 50 to 61, wherein the molecule comprises at least one small globular human protein-derived building block, such as a CKS1-derived building block, and at least one cargo molecule, wherein the cargo molecule is a lipid.
99. The molecule of item 98, wherein the lipid is a short-chain fatty acid.
100. The molecule of item 50 to 61, wherein the molecule comprises at least one small globular human protein-derived building block, such as a CKS1-derived building block, and at least two different cargo molecules.
101. The molecule of item 100, wherein the two different cargo molecules are a cell penetrating peptide, such as CMA-1, and an imaging moiety, such as a fluorophore, e.g. Alexa 647 or pHAb.
102. The molecule of any one of items 76 to 101, wherein the molecule additionally comprises a cargo molecule that is an (in vivo) half-life extending moiety.
103. The molecule of item 102, wherein the cargo is a PEG molecule, an ELNN polypeptide or an albumin-binding polypeptide, preferably an albumin-binding ISVD.
104. The molecule of any of items 102 to 103, wherein the cargo comprises or, alternatively consists of, a polypeptide as defined in any one of SEQ ID NOs.: 50-64 or 106, preferably SEQ ID NOs.: 63 or 106.

1. A molecule comprising at least one protein-based building block, wherein the at least one protein-based building block:
a) comprises at least two conjugation sites or attachment points;
b) has a molecular mass of about 2.5 to about 70 kDa;
c) has a globular three-dimensional (3D) structure;
d) has a solubility of 10 mg/mL or more, measured in an aqueous solution at room temperature, wherein the aqueous solution is citrate buffer or PBS, at pH 7.0 or 7.4;
e) does not specifically bind to any human protein or binds one or more human proteins with a K_{D} value greater than 5×10⁻⁴ mol/litre, as determined by surface plasmon resonance, for instance as described in Ober et al. 2001, Intern. Immunology 13:1551-1559; and
f) does not comprise or consists of an amino acid sequence selected from SEQ ID NO.: 1-34 as depicted on Tables A-1 and A-2 of WO 2016/055656 and/or SEQ ID NO.: 1-12 as depicted on Table A-1 of WO 2010/139808.

2. The molecule of claim 1, wherein one of the at least two, preferably the at least two conjugation site(s) or attachment point(s) are present at a solvent-accessible positions in the protein-based building block;
wherein at least one of the attachment points or conjugation sites, preferably at least two attachment points or conjugation sites, more preferably all of the attachment points or conjugation sites, is (are) an engineered attachment point or conjugation site;
wherein the at least two attachment points or conjugation sites are reactive groups present in the side chain of any amino acid in the protein-based carrier building block, preferably reactive groups present in the side chain of a cysteine and/or in the side chain of a tyrosine, and/or in the side chain of a lysine, and/or in the side chain of a non-natural amino acid;
wherein the at least two conjugation sites are selected from a primary amine, a thiol group, a hydroxyl group, a guanidino group, a carboxyl group and/or a thioether group, preferably from a primary amine and/or a thiol group, more preferably a thiol group;
wherein the at least one protein-based building block does not specifically bind to any non-protein molecule, such as DNA, RNA, lipids or glycans, or binds one or more non-protein molecules with a K_{D} value greater than 5×10⁻⁴ mol/litre;
wherein the at least one protein-based building block comprises at least one cargo attached to at least one of the attachment points or conjugation sites; or
any combination thereof.

3.-7. (canceled)

8. The molecule of claim 1, wherein the protein-based building block is a small globular non-human protein-based building block or a small globular human protein-based building block, preferably wherein small globular non-human protein-based building block is an immunoglobulin single variable domain (ISVD)-based building block, a DARP-in-based building block, an affibody-based building block or an affinity-based building block and wherein the small globular human protein-based building block is a cyclin-dependent kinase subunit 1 (CKS1) protein-based building block.

9. The molecule of claim 8, wherein the ISVD-based building block is derived from a V_{H}, a humanized V_{H}, a human V_{H}, a V_{HH}, a humanized V_{HH} or a camelized V_{H} (derived from a heavy-chain ISVD), preferably derived from an ISVD belonging to the “V_{H}3 class”.

10. The molecule of claim 8, wherein the ISVD-derived building block is derived from RSV001A04 (SEQ ID NO.: 179).

11. The molecule of claim 8, wherein the ISVD-derived building block comprises or, alternatively, consists of SEQ ID NO.: 186:
X₁VX₂LX₃EX₄X₅GX₆X₇X₈X₉X₁₀X₁₁GX₁₂X₁₃X₁₄IX₁₅CX₁₆AX₁₇X₁₈X₁₉X₂₀L
X₂₁X₂₂X₂₃VLGWFRX₂₄AX₂₅X₂₆X₂₇X₂₈X₂₉X₃₀FVAAINX₃₁X₃₂X₃₃X₃₄X₃₅X₃₆
X₃₇X₃₈PX₃₉X₄₀VX₄₁X₄₂X₄₃FX₄₄IX₄₅X₄₆X₄₇X₄₈X₄₉X₅₀X₅₁TGX₅₂LX₅₃MX₅₄
X₅₅LX₅₆X₅₇X₅₈DX₅₉AX₆₀YX₆₁CGAGX₆₂PX₆₃X₆₄X₆₅X₆₆AYX₆₇X₆₈X₆₉X₇₀SY
X₇₁X₇₂X₇₃GX₇₄X₇₅TX₇₆VX₇₇VX₇₈X₇₉X₈₀X₈₁X₈₂
wherein
X₁ (position 1 according to Kabat numbering) can be Glu or any amino acid with a reactive group in its side chain, such as cysteine;
X₂ (position 3 according to Kabat numbering) can be Gln or any amino acid with a reactive group in its side chain, such as cysteine;
X₃ (position 5 according to Kabat numbering) can be Val or any amino acid with a reactive group in its side chain, such as cysteine;
X₄ (position 7 according to Kabat numbering) can be Ser or any amino acid with a reactive group in its side chain, such as cysteine;
X₅ (position 8 according to Kabat numbering) can be Gly or any amino acid with a reactive group in its side chain, such as cysteine;
X₆ (position 10 according to Kabat numbering) can be Gly or any amino acid with a reactive group in its side chain, such as cysteine;
X₇ (position 11 according to Kabat numbering) can be Leu, Val Ser, Met, Trp, Phe, Thr, Gln, Glu, Ala, Arg, Gly, Lys, Tyr, Asn, Pro or Ile, preferably Leu or Val or any amino acid with a reactive group in its side chain, such as cysteine;
X₈ (position 12 according to Kabat numbering) can be Val or any amino acid with a reactive group in its side chain, such as cysteine;
X₉ (position 13 according to Kabat numbering) can be Gln or any amino acid with a reactive group in its side chain, such as cysteine;
X₁₀ (position 14 according to Kabat numbering) can be Ala or any amino acid with a reactive group in its side chain, such as cysteine;
X₁₁ (position 15 according to Kabat numbering) can be Gly or any amino acid with a reactive group in its side chain, such as cysteine;
X₁₂ (position 17 according to Kabat numbering) can be Ser or any amino acid with a reactive group in its side chain, such as cysteine;
X₁₃ (position 18 according to Kabat numbering) can be Leu or any amino acid with a reactive group in its side chain, such as cysteine;
X₁₄ (position 19 according to Kabat numbering) can be Ser or any amino acid with a reactive group in its side chain, such as cysteine;
X₁₅: (position 21 according to Kabat numbering) can be Ser or any amino acid with a reactive group in its side chain, such as cysteine;
X₁₆: (position 23 according to Kabat numbering) can be Ala or any amino acid with a reactive group in its side chain, such as cysteine;
X₁₇: (position 25 according to Kabat numbering) can be Ser or any amino acid with a reactive group in its side chain, such as cysteine;
X₁₈: (position 26 according to Kabat numbering) can be Gly or any amino acid with a reactive group in its side chain, such as cysteine;
X₁₉: (position 27 according to Kabat numbering) can be Gly or any amino acid with a reactive group in its side chain, such as cysteine;
X₂₀: (position 28 according to Kabat numbering) can be Ser or any amino acid with a reactive group in its side chain, such as cysteine;
X₂₁: (position 30 according to Kabat numbering) can be Ser or any amino acid with a reactive group in its side chain, such as cysteine;
X₂₂: (position 31 according to Kabat numbering) can be Asn or any amino acid with a reactive group in its side chain, such as cysteine;
X₂₃: (position 32 according to Kabat numbering) can be Tyr or any amino acid with a reactive group in its side chain, such as cysteine;
X₂₄: (position 39 according to Kabat numbering) can be Gln or any amino acid with a reactive group in its side chain, such as cysteine;
X₂₅: (position 41 according to Kabat numbering) can be Pro or any amino acid with a reactive group in its side chain, such as cysteine;
X₂₆: (position 42 according to Kabat numbering) can be Gly or any amino acid with a reactive group in its side chain, such as cysteine;
X₂₇: (position 43 according to Kabat numbering) can be Lys or any amino acid with a reactive group in its side chain, such as cysteine;
X₂₈: (position 44 according to Kabat numbering) can be Glu or any amino acid with a reactive group in its side chain, such as cysteine;
X₂₉: (position 45 according to Kabat numbering) can be Arg or any amino acid with a reactive group in its side chain, such as cysteine;
X₃₀: (position 46 according to Kabat numbering) can be Glu or any amino acid with a reactive group in its side chain, such as cysteine;
X₃₁: (position 52a according to Kabat numbering) can be Trp or any amino acid with a reactive group in its side chain, such as cysteine;
X₃₂: (position 53 according to Kabat numbering) can be Arg or any amino acid with a reactive group in its side chain, such as cysteine;
X₃₃: (position 54 according to Kabat numbering) can be Gly or any amino acid with a reactive group in its side chain, such as cysteine;
X₃₄: (position 55 according to Kabat numbering) can be Asp or any amino acid with a reactive group in its side chain, such as cysteine;
X₃₅: (position 56 according to Kabat numbering) can be Ile or any amino acid with a reactive group in its side chain, such as cysteine;
X₃₆: (position 57 according to Kabat numbering) can be Thr or any amino acid with a reactive group in its side chain, such as cysteine;
X₃₇: (position 58 according to Kabat numbering) can be Ile or any amino acid with a reactive group in its side chain, such as cysteine;
X₃₈: (position 59 according to Kabat numbering) can be Gly or any amino acid with a reactive group in its side chain, such as cysteine;
X₃₉: (position 61 according to Kabat numbering) can be Pro or any amino acid with a reactive group in its side chain, such as cysteine;
X₄₀: (position 62 according to Kabat numbering) can be Asn or any amino acid with a reactive group in its side chain, such as cysteine;
X₄₁: (position 64 according to Kabat numbering) can be Glu or any amino acid with a reactive group in its side chain, such as cysteine;
X₄₂: (position 65 according to Kabat numbering) can be Gly or any amino acid with a reactive group in its side chain, such as cysteine;
X₄₃: (position 66 according to Kabat numbering) can be Arg or any amino acid with a reactive group in its side chain, such as cysteine;
X₄₄: (position 68 according to Kabat numbering) can be Thr or any amino acid with a reactive group in its side chain, such as cysteine;
X₄₅: (position 70 according to Kabat numbering) can be Ser or any amino acid with a reactive group in its side chain, such as cysteine;
X₄₆: (position 71 according to Kabat numbering) can be Arg or any amino acid with a reactive group in its side chain, such as cysteine;
X₄₇: (position 72 according to Kabat numbering) can be Asp or any amino acid with a reactive group in its side chain, such as cysteine;
X₄₈: (position 73 according to Kabat numbering) can be Asn or any amino acid with a reactive group in its side chain, such as cysteine;
X₄₉: (position 74 according to Kabat numbering) can be Ala or any amino acid with a reactive group in its side chain, such as cysteine;
X₅₀: (position 75 according to Kabat numbering) can be Lys or any amino acid with a reactive group in its side chain, such as cysteine;
X₅₁: (position 76 according to Kabat numbering) can be Asn or any amino acid with a reactive group in its side chain, such as cysteine;
X₅₂: (position 79 according to Kabat numbering) can be Tyr or any amino acid with a reactive group in its side chain, such as cysteine;
X₅₃: (position 81 according to Kabat numbering) can be Gln or any amino acid with a reactive group in its side chain, such as cysteine;
X₅₄: (position 82a according to Kabat numbering) can be Asn or any amino acid with a reactive group in its side chain, such as cysteine;
X₅₅: (position 82b according to Kabat numbering) can be Ser or any amino acid with a reactive group in its side chain, such as cysteine;
X₅₆: (position 83 according to Kabat numbering) can be Ala or any amino acid with a reactive group in its side chain, such as cysteine;
X₅₇: (position 84 according to Kabat numbering) can be Pro or any amino acid with a reactive group in its side chain, such as cysteine;
X₅₈: (position 85 according to Kabat numbering) can be Asp or any amino acid with a reactive group in its side chain, such as cysteine;
X₅₉: (position 87 according to Kabat numbering) can be Thr or any amino acid with a reactive group in its side chain, such as cysteine;
X₆₀: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val or any other amino acid with a reactive group in its side chain, such as cysteine;
X₆₁: (position 91 according to Kabat numbering) can be Tyr or any amino acid with a reactive group in its side chain, such as cysteine;
X₆₂: (position 96 according to Kabat numbering) can be Thr or any amino acid with a reactive group in its side chain, such as cysteine;
X₆₃: (position 98 according to Kabat numbering) can be Leu or any amino acid with a reactive group in its side chain, such as cysteine;
X₆₄: (position 99 according to Kabat numbering) can be Asn or any amino acid with a reactive group in its side chain, such as cysteine;
X₆₅: (position 100 according to Kabat numbering) can be Pro or any amino acid with a reactive group in its side chain, such as cysteine;
X₆₆: (position 100a according to Kabat numbering) can be Gly or any amino acid with a reactive group in its side chain, such as cysteine;
X₆₇: (position 100d according to Kabat numbering) can be Ile or any amino acid with a reactive group in its side chain, such as cysteine;
X₆₈: (position 100e according to Kabat numbering) can be Tyr or any amino acid with a reactive group in its side chain, such as cysteine;
X₆₉: (position 100f according to Kabat numbering) can be Asp or any amino acid with a reactive group in its side chain, such as cysteine;
X₇₀: (position 100g according to Kabat numbering) can be Trp or any amino acid with a reactive group in its side chain, such as cysteine;
X₇₁: (position 101 according to Kabat numbering) can be Asp or any amino acid with a reactive group in its side chain, such as cysteine;
X₇₂: (position 102 according to Kabat numbering) can be Tyr or any amino acid with a reactive group in its side chain, such as cysteine;
X₇₃: (position 103 according to Kabat numbering) can be Trp or any amino acid with a reactive group in its side chain, such as cysteine;
X₇₄: (position 105 according to Kabat numbering) can be Arg or any amino acid with a reactive group in its side chain, such as cysteine;
X₇₅: (position 106 according to Kabat numbering) can be Gly or any amino acid with a reactive group in its side chain, such as cysteine;
X₇₆: (position 108 according to Kabat numbering) can be Gln, Leu, Arg, Pro, Glu, Lys, Ser, Thr, Met, Ala or His; preferably Gln or Leu, or any other amino acid with a reactive group in its side chain, such as cysteine;
X₇₇: (position 110 according to Kabat numbering) can be Thr or any amino acid with a reactive group in its side chain, such as cysteine;
X₇₈: (position 112 according to Kabat numbering) can be Ser or any amino acid with a reactive group in its side chain, such as cysteine;
X₇₉: (position 113 according to Kabat numbering) can be Ser or any amino acid with a reactive group in its side chain, such as cysteine;
X₈₀: is absent or Gly;
X₈₁: is absent or Gly;
X₈₂: is absent or Cys,
or a sequence which has 80% or more identity with SEQ ID NO.: 186, preferably a sequence which has 85% or more, 90% or more, 95% or more, 97% or more or 99% or more sequence identity with SEQ ID NO.: 186, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein,
optionally wherein the ISVD-derived building block comprises or, alternatively, consists of SEQ ID NO.: 206:
X₁ₐVQLVEX₁GGGZ₁VX₂AGGX₃LX₄IX₅CX₆AX₇X_{7b}GX_{7c}LSX₈YVLGWFRQ
APGX₉X₁₀REFVAAINWRGX₁₁ITIGPPX₁₂VEX₁₃RFX₁₄IX₁₅RX₁₆NX₁₇X₁₈
NTGYLQMNX₁₉LAPX_{19b}DTAZ₂YYCGAGTPLNPX₂₀AYIYX₂₁WSYDYWG
X₂₂GTZ3VTVX₂₃SX₂₄X₂₅X₂₆
wherein
X₁ₐ (position 1 according to Kabat numbering) can be Glu or any amino acid with a reactive group in its side chain, such as cysteine;
X₁ (position 7 according to Kabat numbering) can be Ser or any amino acid with a reactive group in its side chain, such as cysteine;
Z₁ (position 11 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂ (position 13 according to Kabat numbering) can be Gln or any amino acid with a reactive group in its side chain, such as cysteine;
X₃ (position 17 according to Kabat numbering) can be Ser or any amino acid with a reactive group in its side chain, such as cysteine;
X₄ (position 19 according to Kabat numbering) can be Ser or any amino acid with a reactive group in its side chain, such as cysteine;
X₅: (position 21 according to Kabat numbering) can be Ser or any amino acid with a reactive group in its side chain, such as cysteine;
X₆: (position 23 according to Kabat numbering) can be Ala or any amino acid with a reactive group in its side chain, such as cysteine;
X₇: (position 25 according to Kabat numbering) can be Ser or any amino acid with a reactive group in its side chain, such as cysteine;
X_{7b}: (position 26 according to Kabat numbering) can be Gly or any amino acid with a reactive group in its side chain, such as cysteine;
X_{7c}: (position 28 according to Kabat numbering) can be Ser or any amino acid with a reactive group in its side chain, such as cysteine;
X₈: (position 31 according to Kabat numbering) can be Asn or any amino acid with a reactive group in its side chain, such as cysteine;
X₉: (position 43 according to Kabat numbering) can be Lys or any amino acid with a reactive group in its side chain, such as cysteine;
X₁₀: (position 44 according to Kabat numbering) can be Glu or any amino acid with a reactive group in its side chain, such as cysteine;
X₁₁: (position 55 according to Kabat numbering) can be Asp or any amino acid with a reactive group in its side chain, such as cysteine;
X₁₂: (position 62 according to Kabat numbering) can be Asn or any amino acid with a reactive group in its side chain, such as cysteine;
X₁₃: (position 65 according to Kabat numbering) can be Gly or any amino acid with a reactive group in its side chain, such as cysteine;
X₁₄: (position 68 according to Kabat numbering) can be Thr or any amino acid with a reactive group in its side chain, such as cysteine;
X₁₅: (position 70 according to Kabat numbering) can be Ser or any amino acid with a reactive group in its side chain, such as cysteine;
X₁₆: (position 72 according to Kabat numbering) can be Asp or any amino acid with a reactive group in its side chain, such as cysteine;
X₁₇: (position 74 according to Kabat numbering) can be Ala or any amino acid with a reactive group in its side chain, such as cysteine;
X₁₈: (position 75 according to Kabat numbering) can be Lys or any amino acid with a reactive group in its side chain, such as cysteine;
X₁₉: (position 82b according to Kabat numbering) can be Ser or any amino acid with a reactive group in its side chain, such as cysteine;
X_{19b}: (position 85 according to Kabat numbering) can be Asp or any amino acid with a reactive group in its side chain, such as cysteine;
Z₂: (position 89 according to Kabat numbering) can be Leu, Val, Ser, Met, Trp, Phe, Thr, Gln, Glu, Ala, Arg, Gly, Lys, Tyr, Asn, Pro or Ile; preferably Leu, Val, Ser or Glu, more preferably Leu or Val;
X₂₀: (position 100a according to Kabat numbering) can be Gly or any amino acid with a reactive group in its side chain, such as cysteine;
X₂₁: (position 100f according to Kabat numbering) can be Asp or any amino acid with a reactive group in its side chain, such as cysteine;
X₂₂: (position 105 according to Kabat numbering) can be Arg or any amino acid with a reactive group in its side chain, such as cysteine;
Z₃: (position 108 according to Kabat numbering) can be Gln, Leu, Arg, Pro, Glu, Lys, Ser, Thr, Met, Ala or His; preferably Gln or Leu;
X₂₃: (position 112 according to Kabat numbering) can be Ser or any amino acid with a reactive group in its side chain, such as cysteine;
X₂₄: is absent or Gly;
X₂₅: is absent or Gly;
X₂₆: is absent or Cys,
or a sequence which has 80% or more identity with SEQ ID NO.: 206, preferably a sequence which has 85% or more, 90% or more, 95% or more, 97% or more or 99% or more sequence identity with SEQ ID NO.: 206, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

12. (canceled)

13. The molecule of claim 8, wherein the DARPin-based building block is derived from the polypeptide as defined in SEQ ID NO.: 187.

14. The molecule of claim 13, wherein at least one protein-based building block comprises, or alternatively, consists of, SEQ ID NO.: 188:
X₁X₂GX₃X₄LLX₅AAX₆X₇X₈X₉X₁₀X₁₁X₁₂VX₁₃X₁₄LMX₁₅X₁₆X₁₇AX₁₈VX₁₉A
X₂₀X₂₁X₂₂X₂₃GX₂₄TPLHLAAX₂₅X₂₆X₂₇X₂₈X₂₉X₃₀IVX₃₁VLLX₃₂X₃₃X₃₄A
X₃₅VX₃₆AX₃₇DX₃₈X₃₉GATPLHLAAX₄₀X₄₁X₄₂X₄₃X₄₄X₄₅IVX₄₆VLLX₄₇X₄₈
X₄₉AX₅₀VX₅₁AX₅₂DX₅₃X₅₄GATPLHX₅₅AAX₅₆X₅₇X₅₈X₅₉X₆₀X₆₁IVX₆₂X₆₃
LX₆₄X₆₅X₆₆X₆₇AX₆₈X₆₉X₇₀AX₇₁DX₇₂X₇₃X₇₄X₇₅TAX₇₆X₇₇ISX₇₈X₇₉X₈₀X₈₁
X₈₂X₈₃X₈₄LAX₈₅X₈₆LX₈₇X₈₈X₈₉X₉₀,
wherein
X₁ can be Asp or any amino acid with a reactive group in its side chain, such as cysteine;
X₂ can be Leu or any amino acid with a reactive group in its side chain, such as cysteine;
X₃ can be Lys or any amino acid with a reactive group in its side chain, such as cysteine;
X₄ can be Lys or any amino acid with a reactive group in its side chain, such as cysteine;
X₅ can be Glu or any amino acid with a reactive group in its side chain, such as cysteine;
X₆ can be Arg or any amino acid with a reactive group in its side chain, such as cysteine;
X₇ can be Ala or any amino acid with a reactive group in its side chain, such as cysteine;
X₈ can be Gly or any amino acid with a reactive group in its side chain, such as cysteine;
X₉ can be Gln or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Arg or any amino acid with a reactive group in its side chain, such as cysteine;
X₁₄ can be Ile or any amino acid with a reactive group in its side chain, such as cysteine;
X₁₅ can be Ala or any amino acid with a reactive group in its side chain, such as cysteine;
X₁₆ can be Asn or any amino acid with a reactive group in its side chain, such as cysteine;
X₁₇ can be Gly or any amino acid with a reactive group in its side chain, such as cysteine;
X₁₈ can be Asp or any amino acid with a reactive group in its side chain, such as cysteine;
X₁₉ can be Asn or any amino acid with a reactive group in its side chain, such as cysteine;
X₂₀ can be His or any amino acid with a reactive group in its side chain, such as cysteine;
X₂₁ can be Asp or any amino acid with a reactive group in its side chain, such as cysteine;
X₂₂ can be Thr or any amino acid with a reactive group in its side chain, such as cysteine;
X₂₃ can be Phe or any amino acid with a reactive group in its side chain, such as cysteine;
X₂₄ can be Phe or any amino acid with a reactive group in its side chain, such as cysteine;
X₂₅ can be Leu or any amino acid with a reactive group in its side chain, such as cysteine;
X₂₆ can be Tyr or any amino acid with a reactive group in its side chain, such as cysteine;
X₂₇ can be Gly or any amino acid with a reactive group in its side chain, such as cysteine;
X₂₈ can be His or any amino acid with a reactive group in its side chain, such as cysteine
X₂₉ can be Leu or any amino acid with a reactive group in its side chain, such as cysteine
X₃₀ can be Glu or any amino acid with a reactive group in its side chain, such as cysteine
X₃₁ can be Glu or any amino acid with a reactive group in its side chain, such as cysteine
X₃₂ can be Lys or any amino acid with a reactive group in its side chain, such as cysteine
X₃₃ can be Asn or any amino acid with a reactive group in its side chain, such as cysteine
X₃₄ can be Gly or any amino acid with a reactive group in its side chain, such as cysteine
X₃₅ can be Asp or any amino acid with a reactive group in its side chain, such as cysteine
X₃₆ can be Asn or any amino acid with a reactive group in its side chain, such as cysteine
X₃₇ can be Asp or any amino acid with a reactive group in its side chain, such as cysteine
X₃₈ can be Ser or any amino acid with a reactive group in its side chain, such as cysteine
X₃₉ can be Tyr or any amino acid with a reactive group in its side chain, such as cysteine;
X₄₀ can be Met or any amino acid with a reactive group in its side chain, such as cysteine;
X₄₁ can be Arg or any amino acid with a reactive group in its side chain, such as cysteine;
X₄₂ can be Gly or any amino acid with a reactive group in its side chain, such as cysteine;
X₄₃ can be His or any amino acid with a reactive group in its side chain, such as cysteine;
X₄₄ can be Leu or any amino acid with a reactive group in its side chain, such as cysteine;
X₄₅ can be Glu or any amino acid with a reactive group in its side chain, such as cysteine;
X₄₆ can be Glu or any amino acid with a reactive group in its side chain, such as cysteine;
X₄₇ can be Lys or any amino acid with a reactive group in its side chain, such as cysteine;
X₄₈ can be Tyr or any amino acid with a reactive group in its side chain, such as cysteine;
X₄₉ can be Gly or any amino acid with a reactive group in its side chain, such as cysteine;
X₅₀ can be Asp or any amino acid with a reactive group in its side chain, such as cysteine;
X₅₁ can be Asn or any amino acid with a reactive group in its side chain, such as cysteine;
X₅₂ can be Ala or any amino acid with a reactive group in its side chain, such as cysteine;
X₅₃ can be Glu or any amino acid with a reactive group in its side chain, such as cysteine;
X₅₄ can be Glu or any amino acid with a reactive group in its side chain, such as cysteine;
X₅₅ can be Leu or any amino acid with a reactive group in its side chain, such as cysteine;
X₅₆ can be Lys or any amino acid with a reactive group in its side chain, such as cysteine;
X₅₇ can be Ala or any amino acid with a reactive group in its side chain, such as cysteine;
X₅₈ can be Gly or any amino acid with a reactive group in its side chain, such as cysteine;
X₅₉ can be His or any amino acid with a reactive group in its side chain, such as cysteine;
X₆₀ can be Leu or any amino acid with a reactive group in its side chain, such as cysteine;
X₆₁ can be Glu or any amino acid with a reactive group in its side chain, such as cysteine;
X₆₂ can be Glu or any amino acid with a reactive group in its side chain, such as cysteine;
X₆₃ can be Val or any amino acid with a reactive group in its side chain, such as cysteine;
X₆₄ can be Leu or any amino acid with a reactive group in its side chain, such as cysteine;
X₆₅ can be Lys or any amino acid with a reactive group in its side chain, such as cysteine;
X₆₆ can be Asn or any amino acid with a reactive group in its side chain, such as cysteine;
X₆₇ can be Gly or any amino acid with a reactive group in its side chain, such as cysteine;
X₆₈ can be Asp or any amino acid with a reactive group in its side chain, such as cysteine;
X₆₉ can be Val or any amino acid with a reactive group in its side chain, such as cysteine;
X₇₀ can be Asn or any amino acid with a reactive group in its side chain, such as cysteine;
X₇₁ can be Gln or any amino acid with a reactive group in its side chain, such as cysteine;
X₇₂ can be Lys or any amino acid with a reactive group in its side chain, such as cysteine;
X₇₃ can be Phe or any amino acid with a reactive group in its side chain, such as cysteine;
X₇₄ can be Gly or any amino acid with a reactive group in its side chain, such as cysteine;
X₇₅ can be Lys or any amino acid with a reactive group in its side chain, such as cysteine;
X₇₆ can be Phe or any amino acid with a reactive group in its side chain, such as cysteine;
X₇₇ can be Asp or any amino acid with a reactive group in its side chain, such as cysteine;
X₇₈ can be Ile or any amino acid with a reactive group in its side chain, such as cysteine;
X₇₉ can be Asp or any amino acid with a reactive group in its side chain, such as cysteine;
X₈₀ can be Asn or any amino acid with a reactive group in its side chain, such as cysteine;
X₈₁ can be Gly or any amino acid with a reactive group in its side chain, such as cysteine;
X₈₂ can be Asn or any amino acid with a reactive group in its side chain, such as cysteine;
X₈₃ can be Glu or any amino acid with a reactive group in its side chain, such as cysteine;
X₈₄ can be Asp or any amino acid with a reactive group in its side chain, such as cysteine;
X₈₅ can be Glu or any amino acid with a reactive group in its side chain, such as cysteine;
X₈₆ can be Ile or any amino acid with a reactive group in its side chain, such as cysteine;
X₈₇ can be Gln or any amino acid with a reactive group in its side chain, such as cysteine;
X₈₈ can be Lys or any amino acid with a reactive group in its side chain, such as cysteine;
X₈₉ can be absent or Leu;
X₉₀ can be absent or Cys
or a sequence which has 80% or more identity with SEQ ID NO.: 188, preferably a sequence which has 85% or more, 90% or more, 95% or more, 97% or more or 99% or more sequence identity with SEQ ID NO.: 188, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein,
optionally wherein the at least one protein-based building block comprises, or alternatively, consists of, SEQ ID NO.: 189,
DLGKX₁LLEAARAGQDDEVRILMANGADVNAHDTFGFTPLHLAALYGHL
X₂IVEVLLKNGAX₃VNAX₄DSYGATPLHLAAMRGHLX₅IVX₆VLLKYGA
X₇VX₈AX₉DEX₁₀GATPLHLAAKAGHLX₁₁IVEVLLKNGAX₁₂VNAQDKFGK
TAFDISIX₁₃NGNEX₁₄LAEILQX₁₅X₁₆X₁₇,
wherein
X₁ can be Lys or any amino acid with a reactive group in its side chain, such as cysteine;
X₂ can be Glu or any amino acid with a reactive group in its side chain, such as cysteine;
X₃ can be Asp or any amino acid with a reactive group in its side chain, such as cysteine;
X₄ can be Asp or any amino acid with a reactive group in its side chain, such as cysteine;
X₅ can be Glu or any amino acid with a reactive group in its side chain, such as cysteine;
X₆ can be Glu or any amino acid with a reactive group in its side chain, such as cysteine;
X₇ can be Asp or any amino acid with a reactive group in its side chain, such as cysteine;
X₈ can be Asn or any amino acid with a reactive group in its side chain, such as cysteine;
X₉ can be Ala or any amino acid with a reactive group in its side chain, such as cysteine;
X₁₀ can be Glu or any amino acid with a reactive group in its side chain, such as cysteine;
X₁₁ can be Glu or any amino acid with a reactive group in its side chain, such as cysteine;
X₁₂ can be Asp or any amino acid with a reactive group in its side chain, such as cysteine;
X₁₃ can be Asp or any amino acid with a reactive group in its side chain, such as cysteine;
X₁₄ can be Asp or any amino acid with a reactive group in its side chain, such as cysteine;
X₁₅ can be Lys or any amino acid with a reactive group in its side chain, such as cysteine;
X₁₆ can be absent or Leu;
X₁₇ can be absent or Cys,
or a sequence which has 80% or more identity with SEQ ID NO.: 189, preferably a sequence which has 85% or more, 90% or more, 95% or more, 97% or more or 99% or more sequence identity with SEQ ID NO.: 189, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

15. (canceled)

16. The molecule of claim 8, wherein the CSK1-derived building block is derived from the polypeptide as defined in SEQ ID NO.: 190.

17. The molecule of claim 16, wherein the at least one protein-based building block comprises, or alternatively, consists of, SEQ ID NO.: 191:
X₁X₂X₃X₄IX₅X₆SX₇X₈X₉X₁₀X₁₁X₁₂X₁₃X₁₄X₁₅X₁₆X₁₇X₁₈VX₁₉LPX₂₀X₂₁X₂₂
AX₂₃X₂₄VX₂₅X_{23b}X_{24b}X_{25b}X₂₆MX₂₇X₂₈X₂₉X₃₀WX₃₁X₃₂LX₃₃VX₃₄QX₃₅X₃₆
X₃₇WX₃₈HX₃₉X₄₀X₄₁X₄₂X₄₃X₄₄X₄₅X₄₆X₄₇ILLFX₄₈X₄₉X₅₀X₅₁X₅₂X₅₃X₅₄X₅₅
X₅₆X₅₇,
wherein
X₁ can be Ser or any amino acid with a reactive group in its side chain, such as cysteine;
X₂ can be His or any amino acid with a reactive group in its side chain, such as cysteine;
X₃ can be Lys or any amino acid with a reactive group in its side chain, such as cysteine;
X₄ can be Gln or any amino acid with a reactive group in its side chain, such as cysteine;
X₅ can be Tyr or any amino acid with a reactive group in its side chain, such as cysteine;
X₆ can be Tyr or any amino acid with a reactive group in its side chain, such as cysteine;
X₇ can be Asp or any amino acid with a reactive group in its side chain, such as cysteine;
X₈ can be Lys or any amino acid with a reactive group in its side chain, such as cysteine;
X₉ can be Tyr or any amino acid with a reactive group in its side chain, such as cysteine;
X₁₀ can be Asp or any amino acid with a reactive group in its side chain, such as cysteine;
X₁₁ can be Asp or any amino acid with a reactive group in its side chain, such as cysteine;
X₁₂ can be Glu or any amino acid with a reactive group in its side chain, such as cysteine;
X₁₃ can be Glu or any amino acid with a reactive group in its side chain, such as cysteine;
X₁₄ can be Phe or any amino acid with a reactive group in its side chain, such as cysteine;
X₁₅ can be Glu or any amino acid with a reactive group in its side chain, such as cysteine;
X₁₆ can be Tyr or any amino acid with a reactive group in its side chain, such as cysteine;
X₁₇ can be Arg or any amino acid with a reactive group in its side chain, such as cysteine;
X₁₈ can be His or any amino acid with a reactive group in its side chain, such as cysteine;
X₁₉ can be Met or any amino acid with a reactive group in its side chain, such as cysteine;
X₂₀ can be Lys or any amino acid with a reactive group in its side chain, such as cysteine;
X₂₁ can be Asp or any amino acid with a reactive group in its side chain, such as cysteine;
X₂₂ can be Ile or any amino acid with a reactive group in its side chain, such as cysteine;
X₂₃ can be Lys or any amino acid with a reactive group in its side chain, such as cysteine;
X₂₄ can be Leu or any amino acid with a reactive group in its side chain, such as cysteine;
X₂₅ can be Pro or any amino acid with a reactive group in its side chain, such as cysteine;
X_{23b} can be Lys or any amino acid with a reactive group in its side chain, such as cysteine;
X_{24b} can be Thr or any amino acid with a reactive group in its side chain, such as cysteine;
X_{25b} can be His or any amino acid with a reactive group in its side chain, such as cysteine;
X₂₆ can be Leu or any amino acid with a reactive group in its side chain, such as cysteine;
X₂₇ can be Ser or any amino acid with a reactive group in its side chain, such as cysteine;
X₂₈ can be Glu or any amino acid with a reactive group in its side chain, such as cysteine;
X₂₉ can be Ser or any amino acid with a reactive group in its side chain, such as cysteine;
X₃₀ can be Glu or any amino acid with a reactive group in its side chain, such as cysteine;
X₃₁ can be Arg or any amino acid with a reactive group in its side chain, such as cysteine;
X₃₂ can be Asn or any amino acid with a reactive group in its side chain, such as cysteine;
X₃₃ can be Gly or any amino acid with a reactive group in its side chain, such as cysteine;
X₃₄ can be Gln or any amino acid with a reactive group in its side chain, such as cysteine;
X₃₅ can be Ser or any amino acid with a reactive group in its side chain, such as cysteine;
X₃₆ can be Gln or any amino acid with a reactive group in its side chain, such as cysteine;
X₃₇ can be Gly or any amino acid with a reactive group in its side chain, such as cysteine;
X₃₈ can be Val or any amino acid with a reactive group in its side chain, such as cysteine;
X₃₉ can be Tyr or any amino acid with a reactive group in its side chain, such as cysteine;
X₄₀ can be Met or any amino acid with a reactive group in its side chain, such as cysteine;
X₄₁ can be Ile or any amino acid with a reactive group in its side chain, such as cysteine;
X₄₂ can be His or any amino acid with a reactive group in its side chain, such as cysteine;
X₄₃ can be Glu or any amino acid with a reactive group in its side chain, such as cysteine;
X₄₄ can be Pro or any amino acid with a reactive group in its side chain, such as cysteine;
X₄₅ can be Glu or any amino acid with a reactive group in its side chain, such as cysteine;
X₄₆ can be Pro or any amino acid with a reactive group in its side chain, such as cysteine;
X₄₇ can be His or any amino acid with a reactive group in its side chain, such as cysteine;
X₄₈ can be Arg or any amino acid with a reactive group in its side chain, such as cysteine;
X₄₉ can be Arg or any amino acid with a reactive group in its side chain, such as cysteine;
X₅₀ can be Pro or any amino acid with a reactive group in its side chain, such as cysteine;
X₅₁ can be Leu or any amino acid with a reactive group in its side chain, such as cysteine;
X₅₂ can be Pro or any amino acid with a reactive group in its side chain, such as cysteine;
X₅₃ can be Lys or any amino acid with a reactive group in its side chain, such as cysteine;
X₅₄ can be Lys or any amino acid with a reactive group in its side chain, such as cysteine;
X₅₅ can be Pro or any amino acid with a reactive group in its side chain, such as cysteine;
X₅₆ can be Lys or any amino acid with a reactive group in its side chain, such as cysteine;
X₅₇ can be Lys or any amino acid with a reactive group in its side chain, such as cysteine,
or a sequence which has 80% or more identity with SEQ ID NO.: 191, preferably a sequence which has 85% or more, 90% or more, 95% or more, 97% or more or 99% or more sequence identity with SEQ ID NO.: 191, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein,
optionally wherein the at least one protein-based building block comprises, or alternatively, consists of, SEQ ID NO.: 205:
SHKQIYYSX₁X₂X₃X₄X₅EEFEYRHVX₆LPKDIAKLVPX₇THLMSESEWRNL
GVQQSX₈GWVHYX₉IHEPEPHILLFRRPLPKKPKX₁₀,
wherein
X₁ can be Asp or any amino acid with a reactive group in its side chain, such as cysteine;
X₂ can be Lys or any amino acid with a reactive group in its side chain, such as cysteine;
X₃ can be Tyr or any amino acid with a reactive group in its side chain, such as cysteine;
X₄ can be Asp or any amino acid with a reactive group in its side chain, such as cysteine;
X₅ can be Asp or any amino acid with a reactive group in its side chain, such as cysteine;
X₆ can be Met or any amino acid with a reactive group in its side chain, such as cysteine;
X₇ can be Lys or any amino acid with a reactive group in its side chain, such as cysteine;
X₈ can be Gln or any amino acid with a reactive group in its side chain, such as cysteine;
X₉ can be Met or any amino acid with a reactive group in its side chain, such as cysteine;
X₁₀ can be Lys or any amino acid with a reactive group in its side chain, such as cysteine,
or a sequence which has 80% or more identity with SEQ ID NO.: 205, preferably a sequence which has 85% or more, 90% or more, 95% or more, 97% or more or 99% or more sequence identity with SEQ ID NO.: 205, provided that the building block has a globular 3D structure, is soluble, has a size (molecular mass) of about 2.5 to about 70 kDa, such as about 2.5 to about 50 kDa, or of about 2.5 to less than 50 kDa, more preferably of about 2.5 to about 30 kDa, such as about 2.5 to about 16 kDa, such as about 5 to about 16 kDa, or about 7 to about 16 kDa, or about 10 to about 16 kDa, and does not specifically bind to any human protein.

18. (canceled)

19. The molecule of claim 1, wherein the at least one protein-based building block comprises or consist of a polypeptide selected from SEQ ID NO.: 80-105, 175, 199, 208 and/or 222-224.

20. The molecule of claim 1, wherein the molecule comprises at least one protein-based building block and at least one further moiety or cargo, preferably wherein the at least one further moiety or cargo is selected from
a) a half-life extending (HLE) moiety, and/or
b) a targeting moiety, preferably an EGFR-targeting moiety such as GE11 peptide; and/or
c) a therapeutic moiety or precursor therefrom;
d) an imaging moiety;
e) a toxic moiety;
f) a nucleic acid, preferably siRNA;
g) vitamins, preferably folate;
h) Toll-like receptor agonists;
i) glycans, preferably bis mannose 6 phosphate (bisM6P) or mannose 6 phosphate (M6P); and/or
j) lipids, preferably short-chain fatty acids;
optionally wherein the at least one cargo is directly attached to the at least one protein-based building block, or wherein the at least one cargo is attached to the at least one protein-based building block through a linker.

21. (canceled)

22. The molecule of claim 20, wherein the cargo is an (in vivo) half-life extending moiety, preferably a PEG molecule, preferably a 1-20 kDa pEG molecule, more preferably a 1-10 kDa PEG molecule, even more preferably a 1-5 kDa PEG molecule, an ELNN polypeptide or an albumin-binding polypeptide;
optionally wherein the albumin-binding polypeptide is an albumin-binding ISVD, wherein preferably the albumin-binding ISVD comprises or, alternatively consists of, a polypeptide as defined in any one of SEQ ID NOs.: 50-64 or 106, preferably SEQ ID NOs.: 63 or 106.

23. (canceled)

24. The molecule of claim 1, wherein the molecule comprises or, alternatively consists of, a polypeptide as defined in any one of SEQ ID NOs.: 107-127, 170-174, 176 or 200.

25. A nucleic acid encoding the molecule and/or the protein-based building block as defined in claim 1.

26. A vector comprising the nucleic acid as defined in claim 25.

27. A composition comprising the molecule as defined in claim 1, such as a pharmaceutical composition.

28. A method for producing a molecule, wherein the method comprises:
a) expressing, in a suitable host cell or host organism or in another suitable expression system, a nucleic acid sequence encoding the at least one protein-based carrier building block and/or the molecule or part of the molecule as defined in claim 1;
b) optionally isolating and/or purifying the at least one protein-based carrier building block and/or the molecule or part of the molecule expressed in a);
c) optionally conjugating one or more (further) cargos to the attachment point(s) or conjugation sites(s) of the protein-based carrier building block.

29. A method for producing a molecule, wherein the method comprises:
a) chemically synthesizing the at least one protein-based carrier building block and/or the molecule or part of the molecule as defined in claim 1, preferably by using solid-phase peptide synthesis;
b) optionally isolating and/or purifying the at least one protein-based carrier building block and/or the molecule or part of the molecule synthesized in a);
c) optionally conjugating one or more (further) cargos to the attachment point(s) or conjugation sites(s) of the protein-based carrier building block.

30. (canceled)

31. A method for prophylactic and/or therapeutic treatment of an autoimmune/inflammatory disease, an infectious disease and/or cancer, such as hematological (blood) and solid tumor cancer disease comprising
administering to a subject in need of such treatment the molecule according to claim 1 or a composition comprising the molecule according to claim 1.

32. (canceled)
